(19) United States
(12) Patent Application Publication
(10) Pub. No.: US 2023/0192701 A1
(43) Pub. Date: Jun. 22, 2023

(54) BICYCLIC KINASE INHIBITORS AND USES THEREOF

(71) Applicant: iOmx Therapeutics AG, Martinsried (DE)

(72) Inventors: Peter SENNHENN, Martinsried (DE); Hannes LOFERER, Martinsried (DE); David BANCROFT, Martinsried (DE); Art KLUGE, Martinsried (DE)

(73) Assignee: iOmx Therapeutics AG, Martinsried (DE)

(21) Appl. No.: 17/921,787

(22) PCT Filed: Apr. 28, 2021
(86) PCT No.: PCT/EP2021/061151

§ 371 Date: Oct. 27, 2022

(30) Foreign Application Priority Data
Apr. 28, 2020 (EP) 20171745.1

(51) Int. Cl. C07D 487/04; G01N 33/574; A61P 35/00

(52) U.S. Cl. CPC C07D 487/04; G01N 33/57484; A61P 35/00

(57) ABSTRACT

The invention relates to kinase inhibitors, in particular inhibitors of protein kinases including the SIK-family, CSF1R, HCK, TEK-family, BRK, ABL, KIT and/or their mutants. Although structurally similar to other bicyclic kinase inhibitors, the kinase inhibitors of the invention are distinctive; possessing a particular class of heterocyclic moiety. Such kinase inhibitors can display one or more certain properties distinct to their structurally similar kinase inhibitors. The kinase inhibitors of the invention or pharmaceutical compositions comprising them may be used in the treatment of a disorder or condition, such as a proliferative disorder, for example, a leukaemia or solid tumour. In particular, these and other structurally related kinase inhibitors may be used in the treatment of a proliferative disorder—such as a mixed phenotype acute leukaemia (MPAL)—characterised by (inter-alia) the presence of MEF2C protein, a human chromosomal translocation at 11q23, and/or a KMT2A fusion oncoprotein. The kinase inhibitors or pharmaceutical compositions of the invention may be used topically to modulate skin pigmentation in a subject, for example to impart UV protection and reduce skin cancer risk.

The invention relates to kinase inhibitors, in particular inhibitors of protein kinases including the SIK-family, CSF1R, HCK, TEK-family, BRK, ABL, KIT and/or their mutants. Although structurally similar to other bicyclic kinase inhibitors, the kinase inhibitors of the invention are distinctive; possessing a particular class of heterocyclic moiety. Such kinase inhibitors can display one or more certain properties distinct to their structurally similar kinase inhibitors. The kinase inhibitors of the invention or pharmaceutical compositions comprising them may be used in the treatment of a disorder or condition, such as a proliferative disorder, for example, a leukaemia or solid tumour. In particular, these and other structurally related kinase inhibitors may be used in the treatment of a proliferative disorder—such as a mixed phenotype acute leukaemia (MPAL)—characterised by (inter-alia) the presence of MEF2C protein, a human chromosomal translocation at 11q23, and/or a KMT2A fusion oncoprotein. The kinase inhibitors or pharmaceutical compositions of the invention may be used topically to modulate skin pigmentation in a subject, for example to impart UV protection and reduce skin cancer risk.

A kinase inhibitor is an enzyme inhibitor that blocks the action of a kinase. A partial, non-limiting, list of kinases includes ABL, AKT, BCR-ABL, BLK, BRK, c-KIT, c-MET, CDK1, CDK2, CDK3, CDK4, CDK5, CDK6, CDK7, CDK8, CDK9, CDK10, cRAF1, CSF1R, CSK, EGFR, ERBB2, ERBB3, ERBB4, ERK, PAK, FES, FGFR1, FGFR2, FGFR3, FGFR4, FGFR5, FGR, FIT-1, FPS, FRK, FYN, HCK, IGF-1R, INS-R, JAK, KDR, LCK, LYN, MEK, p38, PDGFR, PIK, PKC, PYK2, ROS, SIK1, SIK2, SIK3, SRC, TIE, TIE2, TRK and ZAP70. Kinases are enzymes that add a phosphate group to a protein or another organic molecule, and have been shown to be key regulators in most cellular functions including cell-signalling, -proliferation, -differentiation, -metabolism, -survival, -apoptosis, -motility, DNA damage repair etc. Phosphorylation, in particular deregulated signalling due to defective control of protein phosphorylation, is implicated in a wide range of diseases; such as diseases associated with aberrant activity (e.g., increased activity) of a kinase. Such diseases include, but are not limited to, proliferative diseases (e.g., cancers, benign neoplasms, pathological angiogenesis, inflammatory diseases, and autoimmune diseases), as wells as allergies and CNS disorders.

Protein-tyrosine kinases (PTKs) are enzymes that, in conjunction with ATP as a substrate, phosphorylate tyrosine residues in peptides and proteins. PTKs comprise, inter alia, receptor protein-tyrosine kinases (RPTKs), including members of the epidermal growth factor kinase family (e.g., HER1 and HER2), platelet derived growth factor (PDGF), and kinases that play a role in angiogenesis (e.g., TIE2 and KDR); and, in addition, non-receptor protein-tyrosine kinases, including members of the SYK, JAK and SRC kinase families (e.g., SRC, FYN, LYN, LCK and BLK kinases). Protein-serine/threonine kinases (STKs) are enzymes that phosphorylate the oxygen atom of a serine or threonine side-chain in in peptides and proteins. STKs comprise, inter alia, AKT1, Aurora kinases, BRAF, MAP kinases, PLK1, SIK1, SIK2 and SIK3.

Inhibiting protein kinases, and therefore the phosphorylation of a substrate peptide or protein, has been shown to be useful in treating many diseases. For example, afatinib, an ERBB inhibitor, is useful in treating non-small cell lung cancer; axitinib, a VEGFR, PDGFR, and c-KIT inhibitor, is useful in treating renal cell carcinoma; bosutinib, an ABL/BCR-ABL inhibitor, is useful in treating chronic myelogenous leukaemia; cabozantinib, a c-MET and VEGFR2 inhibitor, is useful in treating thyroid cancer; crizotinib, an ALK, HGFR, and c-MET inhibitor, is useful in treating non-small cell lung cancer; dasatinib, an ABL/BCR-ABL, SRC, and c-KIT inhibitor, is useful in treating chronic myelogenous leukaemia; erlotinib, an EGFR inhibitor, is useful in treating non-small cell lung cancer and pancreatic cancer; gefitinib, an EGFR inhibitor, is useful in treating non-small cell lung cancer; imatinib, an ABL/BCR-ABL inhibitor, is useful in treating chronic myelogenous leukaemia; lapatinib, a HER2 inhibitor, is useful in treating breast cancer; nilotinib, an ABL/BCR-ABL inhibitor, is useful in treating chronic myelogenous leukaemia; pazopanib, a VEGFR, PDGFR, and c-KT inhibitor, is useful in treating renal cell carcinoma and soft tissue sarcoma; palbociclib, an inhibitor of CDK4 and CDK6, is useful in treating ER-positive and HER2-negative breast cancer; ponatinib, an ABL/BCR-ABL, BEGFR, PDGFR, FGFR, EPH, SRC, c-KT, RET, TIE2, and FLT3 inhibitor, is useful in treating chronic myelogenous leukaemia and acute lymphoblastic leukaemia; regorafenib, a RET, VEGFR, and PDGFR inhibitor, is useful in treating colorectal cancer and gastrointestinal stromal tumour; ribociclib, an inhibitor of cyclin D1/CDK4 and CDK6, is useful in treating HR-positive, HER2-negative advanced or metastatic breast cancers; ruxolitinib, a JAK inhibitor, is useful in treating myelofibrosis; sorafenib, a VEGFR, PDGFR, BRAF, and c-KIT inhibitor, is useful in treating renal cell carcinoma and hepatocellular carcinoma; sunitinib, a VEGFR and PDGFR inhibitor, is useful in treating renal cell carcinoma, gastrointestinal stromal tumour, and pancreatic neuroendocrine tumour; tofacitinib, a JAK inhibitor, is useful in treating rheumatoid arthritis; vandetanib, a VEGFR, EGFR, RET and BRK inhibitor, is useful in treating thyroid cancer; and vemurafenib, a BRAF inhibitor, is useful in treating malignant melanoma.

In view of the large number of kinases and associated diseases, there is an ever-existing need for new inhibitors selective for various kinases which might be useful in the treatment of related diseases; in particular there remains a need for new kinase inhibitors, pharmaceutical compositions/formulations and uses thereof (including in treatment regimens) for the treatment of diseases associated with aberrant activity of one or more kinases; in particular, there remains a need for new kinase inhibitors: (a) for use in the treatment of proliferative disorders—such as a mixed phenotype acute leukaemia (MPAL)—that are characterised by (inter-alia) the presence of myocyte enhancer factor 2C (MEF2C) protein, a human chromosomal translocation at 11q23, and/or a lysine methyltransferase 2A (KMT2A) fusion oncoprotein; or (b) that are alternatives to existing kinase inhibitors, such as YKL-05-099 (Sundberg et al 2016, ACS Chem Biol 11:2105), as well as HG-9-91-01 and KIN 112 (Clark et al 2012, PNAS 109:16986), all described as being potent inhibitors of salt-inducible kinases (SIKs) (FIG. 1).

One particular kinase inhibitor is YKL-05-099 (3-(2-chloro-6-methylphenyl)-7-((2-methoxy-4-(1-methylpiperidin-4-yl)phenyl)amino)-1-(5-methoxypyridin-2-yl)-3,4-dihydropyrimido[4,5-d]pyrimidin-2(1H)-one: Sundberg et al 2016)), described as a chemical probe for investigating SIK function in-vivo. SIKs were earlier described as promising therapeutic targets for modulating cytokine responses during innate immune activation (Clark et al 2012, PNAS 109:16986; Sundberg et al 2014, PNAS 111:12468), and starting from the pan-SIK inhibitor HG-9-91-01 improved analogues were developed, yielding a novel chemical probe (YKL-05-099) which displayed increased selectivity for SIKs versus other kinases and enhanced pharmacokinetic properties (Sundberg et al 2016). Sundberg and co-workers (2016) described that in-vivo active does of YKL-05-099 recapitulated the effects of SIK inhibition on inflammatory cytokine responses. However, its pharmacological properties led to it being identified only as a chemical probe to investigate SIK function in-vivo, and to further support the development of other SIK inhibitors for the treatment of inflammatory disorders. Based on its pharmacological properties, YKL-05-099 is not a suitable candidate for drug development (neither is HG-9-91-01 or KIN 112).

Accordingly, there is a particular need for new kinase inhibitors useful in the treatment of disorders or condition, such as inflammatory disorders or cancers—especially solid tumours—the mechanism of which is associated with one or more particular kinases (such as SIKs), treatment of which disorders or condition by existing kinase inhibitors such as YKL-05-099 is not possible because of their pharmacological properties. In particular, there is a need for new drug-like kinase inhibitors useful in the treatment of one or more cancers such as breast, lung (e.g., non-small cell), pancreatic or prostate (e.g., castrate or hormone resistant) cancer, as well as melanoma.

There is also an especial need for new kinase inhibitors useful in the treatment of proliferative disorders—such as a mixed phenotype acute leukaemia (MPAL, also known as mixed lineage leukaemia “MLL”)—that are characterised by (inter-alia) the presence of MEF2C protein (such as phosphorylated MEF2C protein and/or MEF2C protein as an active transcription factor), a human chromosomal translocation at 11q23, and/or a KMT2A fusion oncoprotein.

Mixed phenotype acute leukaemia (MPAL)—also known as “mixed lineage leukaemia” (MLL)—is a very aggressive blood cancer that predominantly occurs in paediatric patients and, unlike other types of childhood acute leukaemias, has a dismal prognosis (reviewed by Slany 2009, Haematologica 94:984). One form of MPAL is characterised by a BCR/ABL rearrangement. MPAL with t(9;22)(q34;q11.2) (or BCR/ABL1 rearrangement) is considered as a separate entity (Arber et al 2016, Blood 127:2391). The t(9;22)(q34;q11.2 translocation results in a BCR/ABL1 fusion gene located on the Philadelphia chromosome (Ph), causing a constitutively active BCR/ABL1 tyrosine kinase. Another form of MPAL is characterised by the presence of lysine methyltransferase 2A (KMT2A) fusion proteins (also known as MLL1 fusion proteins) that are the result of chromosomal translocations affecting the KMT2A gene (also known as the MLL1 gene) at 11q23. This KMT2A/MLL rearrangement is the second most frequent genetic lesion in MPAL (MPAL MLL+). These 11q23 translation events juxtapose the amino-terminus of the histone methyltransferase KMT2A with a variety of different (translocation) fusion partners that destroy normal histone methyltransferase function of KMT2A and replace it by heterologous functions contributed by the (translocation) fusion partner. The resulting protein chimeras are transcriptional regulators that take control of other genes normally controlled by KMT2A. In particular, the transcription factor MEF2C can be controlled by KMT2A and is described as an oncogene in childhood acute leukaemias. MEF2C expression is associated with KMT2A fusion gene rearrangement in AML (Schwieger et al 2009, Blood 114:2476), and MEF2C expression defines a subset of AML patients with poor survival outcome (Lazlo et al 2015, J Hematol & Oncol 8:115).

Tarumoto and co-workers (2018, Mol Cell 69:1017) showed that MEF2C activity in AML is driven by SIK3-phosphorylation of HDAC4, and that SIK3 knock-out or chemical inhibition with the small molecule tool compound HG-9-91-01 strongly decreases viability of several MPAL-associated AML cell lines (including MOLM-13 and MV4-11); because cytoplasmic retention of SIK3-phosphorylated HDAC4 regulates MEF2C activity, by preventing nuclear-located (un-phosphorylated) HDAC4 acting as a repressive cofactor of MEF2C, a transcription factor of tumour survival/maintenance genes associated with AML proliferation (FIG. 2). Indeed, recent research demonstrated that SIK3 inhibition with the small molecule tool compound YKL-05-099 —administered intraperitoneally—suppressed AML progression in-vivo (Tarumoto et al 2020, Blood 135:56). However further SIK3 inhibitors remain needed, in particular those with drug-like properties and especially those that can be administered orally, for use in the treatment of proliferative disorders (such as MPAL), especially those that are associated with SIK3-driven MEF2C-controlled expression of cancer survival genes.

Salt-inducible kinases (SIKs) constitute a serine tyrosine kinase subfamily, belonging to the adenosine monophosphate-activated kinase (AMPK) family. Three members (SIK1, -2, and -3) have been identified so far. Amino acid homology of SIK1 with SIK2 and SIK3 is 78% and 68%, respectively, in the kinase domain. The cloning of SIK1 (also known as SIK and SNF1LK), abundantly expressed in the adrenal glands of high-salt, diet-fed rats, led to subsequent cloning of SIK2 (also known as QIK, KIAA0781 and SNF1LK2), mainly expressed in adipose tissues and the rather ubiquitous SIK3 (also known as QSK, KIAA0999 or L19) (Katoh et al. 2004, Mol. Cell. Endocrinol. 217:109). The three SIKs have a similar structure, with an N-terminal kinase domain (catalytic domain), a middle ubiquitin-associated domain (believed important for phosphorylation by LKB1) and a long C-terminal sequence (believed to be a site for further phosphorylation by PKA). However, there are very diverse roles implicated for the various SIKs. For example, various SIKs have been implicated in biological processes as diverse as osteocyte response to parathyroid hormone (Wein et al. 2016, Nature Commun. 7:13176) to induction of SIK1 by gastrin and inhibition of migration of gastric adenocarcinoma cells (Selvik et al. 2014, PLoS ONE 9:e112485). Other potential roles of salt-inducible kinases (in particular SIK3) are described in WO2018/193084A1 (to the present applicant, and published 25 Oct. 2018) furthermore that SIK3 is a gene involved in tumour cell resistance to cell-mediated immune responses, in particular tumour cell resistance to TNF. Recently, SIKs (particularly SIK3) have been demonstrated to also regulate TGFbeta-mediated transcriptional activity and apoptosis, with Hutchinson et al (2010, Cell Death and Disease 11:49) showing that SIK3 expression or activity results in resistance to TGFbeta-mediated apoptosis.

In particular, as well as playing a role in various inflammatory responses (Clark et al 2014; Sundberg et al 2016) and oncology—especially the sensitisation of tumour cells to immune responses (WO2018/193084A1)—it has been known since 2011 that inhibition of SIK2 promotes melanogenesis in B16F10 melanoma cells (Kumagai et al 2011, PLoS ONE 6(10): e26148). It was subsequently described that the pigmentation pathway including in human skin explants can be efficaciously induced by (topical) treatment with SIK inhibitors, including those structurally related to YKL-05-099 (Mujahid et al 2017, Cell Reports 19:2177). Indeed, using such results, it has subsequently been sought to claim methods of increasing (the appearance of) skin pigmentation in a subject by administering topically to the subject skin an effective account of a SIK inhibitor (WO2018/160774), including using kinase inhibitors previously known to be SIK inhibitors (WO2016/023014).

The kinase known as colony-stimulating factor 1 receptor (CSF1R) binds to its ligand CSF1 and the resulting downstream signalling results in differentiation and survival of myeloid cells that express CSF1R receptor. In particular, CSF1-CSF1R signalling is important for the differentiation of macrophages to the more suppressive M2 phenotype (Lenzo et al 2012, Immunol Cell Bio 90:429). Indeed, the presence of CSF1R+ macrophages in tumours correlates with poor survival in various indications including gastric cancer, breast cancer, ovarian cancer, bladder cancer etc. (Zhang et al 2012, PLoS One 7:e50946t). Therefore, targeting CSF1R with either antibodies or small molecule inhibitors has gained increasing attention in treatment of cancer by eliminating or re-educating suppressive M2 macrophages. PLX3397 is one such inhibitor targeting CSF1R and is in clinical development against melanoma, glioblastoma, AML etc. (Cannarile et al 2017, J Immunotherapy Cancer 5:53).

The kinase known as haematopoietic cell kinase (HCK) is a member of the SRC family of cytoplasmic tyrosine kinases (SFKs), and is expressed in cells of the myeloid and B-lymphocyte cell lineages. Excessive HCK activation is associated with several types of leukaemia and enhances cell proliferation and survival by physical association with oncogenic fusion proteins, and with functional interactions with receptor tyrosine kinases. Elevated HCK activity is also observed in many solid malignancies, including breast and colon cancer, and correlates with decreased patient survival rates. HCK enhances the secretion of growth factors and pro-inflammatory cytokines from myeloid cells, and promotes macrophage polarization towards a wound healing and tumour-promoting alternatively activated phenotype. Within tumour associated macrophages, HCK stimulates the formation of podosomes that facilitate extracellular matrix degradation, which enhance immune and epithelial cell invasion. By virtue of functional cooperation between HCK and bona fide oncogenic tyrosine kinases, excessive HCK activation can also reduce drug efficacy and contribute to chemo-resistance, while genetic ablation of HCK results in minimal physiological consequences in healthy mice. Given its known crystal structure, HCK therefore provides an attractive therapeutic target to both, directly inhibit the growth of cancer cells, and indirectly curb the source of tumour-promoting changes in the tumour microenvironment (Poh et al 2015, Oncotarget 6:15742).

The Tec family has emerged as a subfamily of non-receptor tyrosine kinases, consisting of TEC, Bruton's tyrosine kinase (BTK), Interleukin-2-inducible T-cell kinase (ITK), BMX, and Tyrosine-protein kinase TXK (also known as resting lymphocyte kinase: RKL), all of which are importantly involved in the lymphocyte signalling pathways, in particular as regulators of T-helper-cell differentiation as (Schwartzberg et al 2005, Nature Reviews Immunology 5:284).

BTK is a non-receptor kinase that plays a crucial role in oncogenic signalling that is critical for proliferation and survival of leukemic cells in many B cell malignancies (Singh et al 2018, Molecular Cancer 17:57). BTK was initially shown to be defective in the primary immunodeficiency X-linked agammaglobulinemia (XLA) and is essential both for B cell development and function of mature B cells. More recently, small-molecule inhibitors of this kinase have shown excellent anti-tumour activity, first in animal models and subsequently in clinical studies. In particular, the orally administered irreversible BTK inhibitor ibrutinib (PCI-32765) is associated with high response rates in patients with relapsed/refractory chronic lymphocytic leukaemia (CLL) and mantle-cell lymphoma (MCL). Moreover, BTK functions in several myeloid cell populations representing important components of the tumour microenvironment. As a result, there is currently a considerable interest in BTK inhibition as an anti-cancer therapy, not only in B cell malignancies but also in solid tumours. Efficacy of BTK inhibition as a single agent therapy is strong, but resistance may develop, fuelling the development of combination therapies that improve clinical responses. Therefore, this is a need for alterative inhibitors of BTK.

ITK is an intracellular tyrosine kinase expressed in T-cells. The protein is thought to play a role in T-cell proliferation and differentiation (Kosaka et al 2006, Trends Immunol 27:453), and is functionally important for the development and effector function of Th2 and Th17 cells (Gomez-Rodriguez Jet al 2011, FEBS 278:198). Recently, ITK inhibitors have been compared and investigated for their potential for combination therapies for T-cell lymphoma (Mamand et al 2018, Sci Rep 8:14216).

TXK is a non-receptor tyrosine kinase that plays a redundant role with ITK in regulation of the adaptive immune response. It acts as a Th1 cell-specific transcription factor and Regulates IFN-gene transcription (Takeba et al 2002, J Immun 168:2365; and. like ITK, is bound by a T cell-specific adapter protein (RIBP) that regulated T cell activation (Rajagopal et al 1999, J Exp Med 190:1657).

It was discovered that malignantTcells in the majority of patients display ectopic expression of the B-lymphoid tyrosine kinase (BLK), a member of the Src kinase family (Krejsgaard et al 2009, Blood 113:5896). Importantly, gene knockdown experiments showed that BLK promoted the proliferation of malignant T cells from cutaneous T-cell lymphoma (CTCL) patients. Indeed, BLK is described as an oncogene and has been investigated as a potential target for therapy with dasatinib in CTCL (Peterson et al 2014, Leukemia 28:2109).

Tumour-associated macrophages (TAMs) are an abundant cell type in the tumour microenvironment. These macrophages serve as a promising target for treatment of cancer due to their roles in promoting cancer progression and simultaneous immunosuppression. The TAM receptors (TYRO3, AXL and MERTK) are promising therapeutic targets on TAMs. Post-efferocytosis, macrophages are polarised to a pro-tumour M2-like phenotype and secrete increased levels of immunosuppressive cytokines. Since M2 polarisation and efferocytosis are tumour-promoting processes, the TAM receptors on macrophages serve as exciting targets for cancer therapy. Current TAM receptor-directed therapies in preclinical development and clinical trials may have anti-cancer effects though impacting macrophage phenotype and function in addition to the cancer cells (Myers et al 2019, Molec Cancer 18: 94).

The expression of the tyrosine kinase ZAP70 (member of the Syk tyrosine kinase family) in CLL cells is associated with shorter overall survival in CLL patients (Rassenti et al 2008, Blood 112:1923). The TK inhibitor gefitinib has been shown to be effective at induce apoptosis in acute myeloid leukaemia through inhibition of SYK, and has been shown to preferentially induce cell death in ZAP70-expressing primary human CLL cells, representing the likelihood that other TK inhibitors may be effective targeted treatments for ZAP70+CLL cells (Dielschneider et al 2014, Cell Death & Disease 5:e1439).

Breast tumour kinase (BRK)/protein tyrosine kinase 6, has various oncogenic roles in breast cancer cell proliferation, survival, and migration, and is overexpressed in approximately 80% of breast cancers (Barker et al 1997, Oncogene 15:799). Despite this, BRK have been little explored as possible therapeutic tools. Jiang et al (2017, Cancer Res 77:175) describe prognostic significance of BRK in breast cancer, and provide preclinical proof of concept for targeting of BRK in breast cancer.

Hence there still remains a need for new kinase inhibitors, in particular those that exhibit drug like properties (especially those suitable for oral administration) and that inhibit one or more kinases, including any of those selected from SIK3, SIK2, SIK1, ABL/BCR-ABL, KIT, NEK2, BRAF. CSF1R, HCK, TEC-family kinases (eg BTK, TXK or ITK), AXL, BLK, TYRO3, MERTK, ZAP70, SYK, EGFR, and/or BRK, and/or that exhibit a different profile of kinases to the kinases inhibited by YKL-05-099, in particular. For example, new kinase inhibitors which: (i) inhibit and/or are more specific to key disease-related kinases such as SIK3 (and e.g., ABL/BCR-ABL, KIT, NEK2, BRAF. CSF1R, HCK, TEC-family kinases (eg BTK, TXK or ITK), AXL, BLK, TYRO3, MERTK, ZAP70, SYK, EGFR, BRK and/or KIT), relative to other kinases, than the specificity shown by YKL-05-099 to one or more such other kinases; (ii) inhibit key disease- or side-effect-related kinases in a different profile than YKL-05-099 (e.g. to KIT and/or LCK); and/or (iii) inhibit one or more mutant of a disease-related kinase, in particular a mutant that is resistant to one or other kinase inhibitor, such as mutants of ABL/BCR-ABL or KIT.

WO00/24744 discloses bicyclic nitrogen heterocycles are, almost exclusively those with phenyl substituents at R2 of formula (I) therein, and their in-vitro activity as inhibitors of T cell tyrosine kinase p56′^{ck} (also knowns LCK).

WO2014/089913 discloses bicyclic compounds functioning as inhibitors of Bruton tyrosine kinase (BTK), such compounds possessing a particular class of substituents at ring A and/or B of formula (I) therein.

CN107286130 discloses selective inhibitors of FGFR4 kinase, including those of formula (V) therein, such compounds possessing a specific substituent at ring A, and in particular other specific substituents at the pyrimidine (see, for example formula (IV) therein).

WO2015/006492 describes the synthesis of various compounds structurally similar to YKL-05-099 (as well as similar to HG-9-91-01 and KIN112), and their biological activity and properties as kinase inhibitors, especially of BTK and EGFR. WO2016/014551 describes macrocyclic versions of such compounds and their inhibitory activity over a wide range of protein kinases (see Table 2 thereof). In particular, the SIK-inhibitory activity of such classes of compounds is disclosed in WO2016/023014 (see formulae (I) and (III) therein), and their further use as SIK inhibitors to increase (the appearance of) skin pigmentation in a subject is disclosed in WO2018/160774.

Sundberg et al (2016, ACS Chem Biol 11:2105) describes YKL-05-099 (and similar compounds) as having pharmacologically improved properties compared to HG-9-91-01, however with such improvements coming at the cost of loss in inhibition-potency of YKL-05-099 to SIK3.

Jiang et al (2017, Cancer Res 77:175) describe XMU-MP-2 as a potent BRK inhibitor.

WO 2018/193084 (to the present applicant, and published 25 Oct. 2018) discloses a single dasatinib variant carrying a pyridinyl moiety and its uses. Dasatinib has been described to be an inhibitor of salt-inducible kinases (WO2018/193084; Ozanne et al 2015, Biochem J 465:1039). Co-pending application PCT/EP2019/078751 (to the present applicant) discloses further dasatinib variants carrying other heterocyclic moieties, and in particular variants that carry a thiazoyl moiety. Beutner et al (2018, Org Lett 20:4218) describes a method of forming challenging amide bonds, including those to certain pyridines pyrazines and pyrimidines. Pennington et al (2017, J Med Chem 60:3552) describes that the replacement of a CH group with a N atom in aromatic and heteroaromatic ring systems can have effects on molecular and physiological properties. However, making such a substitution has been empirically shown to result in improved potency statistically no better than mere chance: a matched molecular pair analysis (MMPA) of internal data at Abbott (Hajduk & Sauer 2008, J Med Chem 51:553) found that, as with most substituent replacements, there is an approximate equal probability of increasing or decreasing potency by exchanging CH groups and N atoms. Indeed, this analysis further revealed that the probability for realising a 10-fold increase in potency with such replacements is less than 1 in 10 and that for achieving a 100-fold is less than 1 in 100; similar to the probabilities observed when investigating the effect of such replacements to improve binding affinity (Hu et al 2014, F1000Research 3:36; de la Vega de Leon et al 2014, MedChemComm 5:64).

Accordingly, it is one object of the present invention to provide one or more kinase inhibitors (eg, an inhibitor of any of the kinases described above, such as SIK3, HCK, TEK-family, BRK and/or CSF1R kinases) that have one or more properties (such as those shown by in-vitro and/or in-vivo assays) that address one or more of these or other problems. In other objects, the present invention provides an alternative and/or improved kinase inhibitor to YKL-05-099 (or one or other kinase inhibitor, such as those described herein). For example, it would be desired to provide kinase inhibitors that are more potent inhibitors of SIK2 and/or SIK3 than YKL-05-099; and/or that are more selective inhibitors of SIK3 (eg, over SIK2 or other kinases) than YKL-05-099. Also, for example, a kinase inhibitor that can exhibit one or more functional (e.g., kinase selectivity), ADMET, PK and/or pharmacological properties that are different to, and/or are improved compared to, YKL-05-099 (or one or other kinase inhibitor, such as those described herein), would be advantageous. In particular, it would be advantageous to provide inhibitors of one or more SIK-family kinases that have (more) drug-like properties and especially those that can be administered orally, for use in the treatment of a proliferative disorder (such as MPAL) characterised, inter-alia, by the presence of MEF2C protein (such as phosphorylated MEF2C protein and/or MEF2C protein as an active transcription factor), a human chromosomal translocation at 11q23, and/or a KMT2A fusion oncoprotein. Also advantageous, would be to provide inhibitors of one or more SIK-family kinases that are useful for administration (eg topically) to a subject for increasing (the appearance of) skin pigmentation in such subject. An object underlying the present invention is solved by the subject matter as disclosed or defined anywhere herein, for example by the subject matter of the attached claims.

### SUMMARY OF THE INVENTION

In a first aspect, and as may be further described, defined, claimed or otherwise disclosed herein, the present invention provides a compound selected from the group consisting of a kinase inhibitor of the formula (I)

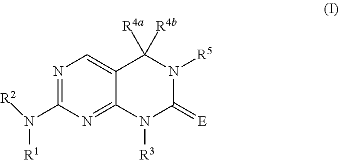

and solvates, salts, N-oxides, complexes, polymorphs, crystalline forms, racemic mixtures, diastereomers, enantiomers, tautomers, conformers, isotopically labeled forms, prodrugs, and combinations thereof; wherein R¹, R², R³, R^{4a}, R^{4b}, R⁵ and E are as defined herein.

In a second aspect, the present invention provides a pharmaceutical composition comprising a compound of the first aspect.

In a third aspect, the present application provides a compound of the first aspect or a pharmaceutical composition of the second aspect for use in therapy.

In a fourth aspect, the present application provides a compound of the first aspect or a pharmaceutical composition of the second aspect for use in a treatment of a proliferative disorder in a subject

in a fifth aspect, the present invention relates to a compound for use, or a pharmaceutical composition for use, in a treatment of a proliferative disorder in a subject, the treatment comprising administering the compound or the pharmaceutical composition to the subject, wherein the compound is selected from a compound of the first aspect, and wherein the proliferative disorder is selected from one or more of (α) to (γ):

(α) a proliferative disorder characterised by (or cells involved with the proliferative disorder characterised by) the presence of (or an amount of) myocyte enhancer factor 2C (MEF2C) protein, such as of phosphorylated MEF2C protein and/or of MEF2C protein as an active transcription factor; preferably wherein the proliferative disorder is further characterised by (or cells involved with the proliferative disorder are further characterised by) the presence of (or an amount of phosphorylated histone deacetylase 4 (HDAC4) protein, such as of HDAC4 protein phosphorylated by SIK3; and/or
(β) a proliferative disorder characterised by: (i) the presence of a human chromosomal translocation at 11q23; (ii) the presence of a rearrangement of the lysine methyltransferase 2A (KMT2A) gene; (iii) the presence of (or an amount of) an KMT2A fusion oncoprotein; and/or (iv) the presence of a mutation in the K-RAS proto-oncogene GTPase (KRAS) gene and/or in the RUNX family transcription factor 1 (RUNX1) gene; and/or
(γ) a mixed phenotype acute leukaemia (MPAL). In a related aspect, the present invention provides a method for the treatment of a proliferative disorder in a subject, comprising administering to the subject a compound or pharmaceutical composition as defined in the fifth aspect, wherein the proliferative disorder is as defined in the fifth aspect.

The invention also provides further aspects which are disclosed herein.

### BRIEF DESCRIPTION OF THE FIGURES

The figures show:

FIG. 1: depicts the chemical structures of the prior-art SIK inhibitors YKL-05-099 (3-(2-chloro-6-methylphenyl)-7-((2-methoxy-4-(1-methylpiperidin-4-yl)phenyl)amino)-1-(5-methoxypyridin-2-yl)-3,4-dihydropyrimido[4,5-d]pyrimidin-2(1H)-one; compound PY1), HG-9-91-01 (compound PH1) and KIN112 (compound PK1).

FIG. 2: depicts a schematic of SIK3-mediated control of the expression of survival/maintenance genes (a) by phosphorylated myocyte enhancer factor 2C (MEF2C) transcription factor. Over expression of MEF2C is associated with the presence of fusion of the lysine methyltransferase 2A (KMT2A) protein (previously known as “MLL”; (b)), typically brought about by a human chromosomal translocation at 11q23. MEF2C activity is controlled by presence in the nucleus of HDAC4 acting, as a repressive co-factor, whose retention in the cytoplasm is brought about by its phosphorylation by SIK3. Nuclear entry and presence (c) of un-phosphorylated HDAC4 (and hence reduction of expression of tumour survival/maintenance genes (a) by inhibition of the transcription-factor activity of MEF2C) can be brought about by inhibition of SIK3 by a compound disclosed herein (d).

FIG. 3: depicts (A) various compounds of Formula (I), including the kinase inhibitors AA1 to AA13; (B) various macrocyclic compounds of Formula (VIII).

FIG. 4: depicts the chemical structures of: (A) dasatinib (compound A8), N-(2-chloro-6-methylphenyl)-2-((6-(4-(2-hydroxyethyl)piperazin-1-yl)-2-methylpyrimidin-4-yl)amino)thiazole-5-carboxamide. Also depicted are various other kinases inhibitors with diverse heterocyclic moieties, with an expected equivalent kinase back pocket-binding function as R⁶ for the compounds of Formula (I): (B) the kinase inhibitor B3, N-(4-chloro-2-methylpyridin-3-yl)-2-((6-(4-(2-hydroxyethyl)piperazin-1-yl)-2-methylpyrimidin-4-yl)amino)thiazole-5-carboxamide, having a pyridyl R6-equivilent back-pocket moiety; (C) certain other kinase inhibitors with diverse R⁶-equivilent back-pocket moieties; and (D) certain further kinase inhibitors with diverse thiophene-based R⁶-equivilent back-pocket moieties.

FIG. 5: depicts scatter plots comparing residual kinase activity for over 335 kinases for various pairs of concentrations of tested compounds. X axes=residual activity at 0.1 nM compound; Y axes=residual activity at 1 nM tested compound. (A) compound PY1; (B) compound AA1; (C) compound AA3; (D) compound AA21; and (E) compound AA5; The few outlying data points for particular kinases are marked: (A) X=SGK1, Y=SNARK; and (E) X=EPHA6, Y=EPHA3.

FIG. 6: depicts scatter plots comparing residual kinase activity for 335 kinases for compound AA1 (Y axes) compared to the prior art compound PY1 (X axes) across the full range of residual activity (A) and less than 25% residual activity (B). Circled are the relative residual activities for kinases BLK, SIK3 and TEC. Both compounds tested at 1 nM.

FIG. 7: depicts scatter plots comparing residual kinase activity for 335 kinases for compound AA11 (Y axes) compared to the prior art compound PY1 (X axes) across the full range of residual activity (A), and less than 25% residual activity (B). Circled in (A) are the relative residual activities for kinases BRAF, NEK2, PRK2 and PKC (solid line), and BMX and TEC (broken line); and in (B) KIT, RIPK2, ABL2 and PDGF-alpha (solid line) and BTK (broken line). Both compounds tested at 1 nM.

FIG. 8: depicts scatter plots comparing residual kinase activity for 335 kinases for compound AA3 (Y axes) compared to the prior art compound PY1 (X axes) across the full range of residual activity with both compounds tested at 1 nM (A), and less than 25% residual activity with both compounds tested at 0.1 nM (B). Circled in (A) are the relative residual activities for kinases TAOK2, SYK. TYRO3, ACVR2B, MEKK2, AXL, ITK, MAP3K11, TRKA, MERTK, ZAP70, and MEKK2 (solid line), and SRMS, NLK, RIPK5, LTK and ALK (broken line); and in (B) CSF1R, HCK, TXK, YES, LCK, SRC, EPHA1 and FGR (solid line).

FIG. 9: depicts scatter plots comparing residual kinase activity for 335 kinases for compound AA5 (Y axes) compared to the prior art compound PY1 (X axes) across the full range of residual activity (A), and less than 50% residual activity (B). Circled in (B) are the relative residual activities for kinases TXK, ERBB4, EPHB1, FRK, BRK, EPHA4, ACK1, EGFR, EPHA1, SIK1 and CSF1R (solid line), and FYN, BTK, EPHB2, LCK and CSK, (broken line). Both compounds tested at 1 nM.

FIG. 10: depicts TNF-dependent dose-response curves for compounds AA6 (A) and AA7(B) compared to the prior art compound PY1 (C), tested against the human HCT116 cell lines at different (human) TNF concentrations (rHuTNF concentrations: x=0 ng/ml, y=2.5 ng/ml and y=10 ng/ml). Vertical bars: normalised viability with no compound at the x-, y- and z-indicated concentration of rHuTNF). Y axes=Viability (normalised to no compound); X axes=compound concentration (nM).

FIG. 11: depicts scatter plots of: X-axes=EC50 of 100 ng/mL rMuTNF-sensitisation by compounds of Formula (I)) (normalised against the -TNF EC50); and Y axes=IC50 (nM) for SIK3 (A) and SIK2 (B) for each compound against the MC38 cell line.

FIG. 12: depicts PK characteristics of compounds PY1 and AA1 after 30 mg/kg po and 1 mg/kg iv administration. Circles=PY130 mg/kg po; Squares=AA130 mg/kg po; Triangles=PY 11 mg/kg iv; Inverted triangles=AA1 1 mg/kg iv. Y axis=Compound plasma concentration (ng/ml); X axis=Time (h).

FIG. 13: depicts a classification of all yet known KMT2A fusion translocation partner genes (TPGs) by disease (adapted from FIG. 3 Meyer et al 2018). All TPGs are grouped by their diagnosed disease type. Such genes have been diagnosed in ALL, t-ALL, t-AML, AML, T-ALL, MLL, bilineal acute leukaemia (BAL), MDS, t-MDS, chronic myelogenous leukaemia (CML), t-CML, juvenile myelomonocytic leukaemia (JMML) and lymphoma. Genes in the intersection belong to two different groups. Bold-marked TPGs are the most frequent ones.

### DETAILS OF THE PRESENT INVENTION

The present invention, and particular non-limiting aspects and/or embodiments thereof, can be described in more detail as follows.

Although the present invention may be further described in more detail, it is to be understood that this invention is not limited to the particular methodologies, protocols and reagents described herein as these may vary. It is also to be understood that the terminology used herein is for the purpose of describing particular embodiments only, and is not intended to limit the scope of the present invention which will be limited only by what is described, defined or otherwise disclosed herein, in particular in any itemised embodiments or the appended claims.

Herein, certain elements of the present invention are described in more detail. These elements are listed with specific embodiments, however, it should be understood that they may be combined in any manner and in any number to create additional embodiments. The variously described examples and preferred embodiments should not be construed to limit the present invention to only the explicitly described embodiments. This description of this application should be understood to support and encompass embodiments which combine the explicitly described embodiments with any number of the disclosed and/or preferred elements. Furthermore, any permutations and combinations of all described elements in this application should be considered disclosed by the description of the present application unless the context indicates otherwise. For example, if in one embodiment of the compound of the invention L is a bond and in another embodiment of the compound of the invention R³ is Me, then in a preferred embodiment of the compound of the invention, L is a bond and R³ is Me, or if in one embodiment of the use of a compound of the invention the subject is an adult human and in another embodiment of the use of a compound of the invention the proliferative disorder is prostate cancer, then in a preferred embodiment of the use of a compound of the invention, the subject is an adult human and the proliferative disorder is prostate cancer.

### General Definitions

Unless defined otherwise, all technical and scientific terms used herein have the same meanings as commonly understood by one of ordinary skill in the art.

Preferably, the terms used herein are defined as described in “A multilingual glossary of biotechnological terms: (IUPAC Recommendations)”, H. G. W. Leuenberger, B. Nagel, and H. Kolbl, Eds., Helvetica Chimica Acta, CH-4010 Basel, Switzerland, (1995).

The practice of the present invention will employ, unless otherwise indicated, conventional methods of chemistry, biochemistry, and recombinant DNA techniques which are explained in the literature in the field (cf., e.g., Molecular Cloning: A Laboratory Manual, 2nd Edition, I Sambrook et al. eds., Cold Spring Harbor Laboratory Press, Cold Spring Harbor 1989).

Throughout this specification and the claims which follow, unless the context requires otherwise, the word “comprise”, and variations such as “comprises” and “comprising”, will be understood to imply the inclusion of a stated member, integer or step or group of members, integers or steps but not the exclusion of any other member, integer or step or group of members, integers or steps. The term “consisting essentially of” means excluding other members, integers or steps of any essential significance or group of members, integers or steps of any essential significance. For example, a pharmaceutical composition consisting essentially of the members/components as defined herein (such as a compound as defined in any of the aspects of the invention and optionally one additional therapeutic agent) would exclude further therapeutic agents (besides the compound as defined in any of the aspects of the invention and the optional one additional therapeutic agent) but would not exclude contaminants (e.g., those from the isolation and purification method) in trace amounts (e.g., the amount of the contaminant (preferably the amount of all contaminants present in the composition) is less than 5% by weight, such as less than 4% by weight, 3% by weight, 2% by weight, 1% by weight, 0.5% by weight, 0.4% by weight, 0.3% by weight, 0.2% by weight, 0.1% by weight, 0.05% by weight, with respect to the total composition) and/or pharmaceutically acceptable excipients (such as carriers, e.g., phosphate buffered saline, preservatives, and the like). The term “consisting of” means excluding all other members, integers or steps of significance or group of members, integers or steps of significance. For example, a pharmaceutical composition consisting of the members/components as defined herein (such as a compound as defined in any of the aspects of the invention, one excipient, and optionally one additional therapeutic agent) would exclude any other compound (including a second or further excipient) in an amount of more than 2% by weight (such as any other compound in an amount of more than 1% by weight, more than 0.5% by weight, more than 0.4% by weight, more than 0.3% by weight, more than 0.2% by weight, more than 0.1% by weight, more than 0.09% by weight, more than 0.08% by weight, more than 0.07% by weight, more than 0.06% by weight, more than 0.05% by weight, more than 0.04% by weight, more than 0.03% by weight, more than 0.02% by weight, more than 0.01% by weight) with respect to the total composition. The term “comprising” encompasses the term “consisting essentially of” which, in turn, encompasses the term “consisting of”. Thus, at each occurrence in the present application, the term “comprising” may be replaced with the term “consisting essentially of” or “consisting of”. Likewise, at each occurrence in the present application, the term “consisting essentially of” may be replaced with the term “consisting of”.

Where used herein, “and/or” is to be taken as specific disclosure of each of the two specified features or components with or without the other. For example, “X and/or Y” is to be taken as specific disclosure of each of (i) X, (ii) Y, and (iii) X and Y, just as if each is set out individually herein.

In the context of the present invention, the terms “about” and “approximately” are used interchangeably and denote an interval of accuracy that the person of ordinary skill will understand to still ensure the technical effect of the feature in question. The term typically indicates deviation from the indicated numerical value by +5%, +4%, +3%, +2%, ±1%, +0.9%, +0.8%, +0.7%, +0.6%, +0.5%, +0.4%, +0.3%, +0.2%, +0.1%, +0.05%, and for example +0.01%. As will be appreciated by the person of ordinary skill, the specific such deviation for a numerical value for a given technical effect will depend on the nature of the technical effect. For example, a natural or biological technical effect may generally have a larger such deviation than one for a man-made or engineering technical effect.

The terms “a”, “an” and “the” and similar references used in the context of describing the invention (especially in the context of the claims) are to be construed to cover both the singular and the plural, unless otherwise indicated herein or clearly contradicted by the context.

Recitation of ranges of values herein is merely intended to serve as a shorthand method of referring individually to each separate value falling within the range. Unless otherwise indicated herein, each individual value is incorporated into the specification as if it were individually recited herein.

All methods described herein can be performed in any suitable order unless otherwise indicated herein or otherwise clearly contradicted by the context.

The use of any and all examples, or exemplary language (e.g., “such as”), provided herein is intended merely to better illustrate the invention and does not pose a limitation on the scope of the invention otherwise claimed. No language in the specification should be construed as indicating any non-claimed element essential to the practice of the invention.

Several documents are cited throughout the text of this specification. Each of the documents cited herein (including all patents, patent applications, scientific publications, manufacturer's specifications, instructions, etc.), whether supra or infra, are hereby incorporated by reference in their entirety. Nothing herein is to be construed as an admission that the invention is not entitled to antedate such disclosure by virtue of prior invention.

The terms “of the [present] invention”, “in accordance with the [present] invention”, “according to the [present] invention” and the like, as used herein are intended to refer to all aspects and embodiments of the invention described and/or claimed herein.

It is to be understood that the application of the teachings of the present invention to a specific problem or environment, and the inclusion of variations of the present invention or additional features thereto (such as further aspects and embodiments), will be within the capabilities of one having ordinary skill in the art in light of the teachings contained herein.

Unless context dictates otherwise, the descriptions and definitions of the features set out above or below are not limited to any particular aspect or embodiment of the invention and apply equally to all aspects and embodiments that are described.

The term “alkyl” refers to a monoradical of a saturated straight or branched hydrocarbon. Preferably, the alkyl group comprises from 1 to 12 (such as 1 to 10) carbon atoms, i.e., 1, 2, 3, 4, 5, 6, 7, 8, 9, 10, 11, or 12 carbon atoms (such as 1, 2, 3, 4, 5, 6, 7, 8, 9, or 10 carbon atoms), more preferably 1 to 8 carbon atoms, such as 1 to 6 or 1 to 4 carbon atoms. Exemplary alkyl groups include methyl (Me), ethyl (Et), propyl, iso-propyl (also called 2-propyl or 1-methylethyl), butyl, iso-butyl, tert-butyl, n-pentyl, iso-pentyl, sec-pentyl, neo-pentyl, 1,2-dimethyl-propyl, iso-amyl, n-hexyl, iso-hexyl, sec-hexyl, n-heptyl, iso-heptyl, n-octyl, 2-ethyl-hexyl, n-nonyl, n-decyl, n-undecyl, n-dodecyl, and the like. A “substituted alkyl” means that one or more (such as 1 to the maximum number of hydrogen atoms bound to an alkyl group, e.g., 1, 2, 3, 4, 5, 6, 7, 8, 9, or up to 10, such as between 1 to 5, 1 to 4, or 1 to 3, or 1 or 2) hydrogen atoms of the alkyl group are replaced with a substituent other than hydrogen (when more than one hydrogen atom is replaced the substituents may be the same or different). Preferably, the substituent other than hydrogen is a 1^{st} level substituent, a 2^{nd} level substituent, or a 3^{rd} level substituent as specified herein, such as halogen, —OH, —NH₂, —NHCH₃, —N(CH₃)₂, —CN, —OCH₃, —OCF₃, or optionally substituted aryl. Examples of a substituted alkyl include trifluoromethyl, difluoromethyl, fluoromethyl, 2,2,2-trichloroethyl, 2-hydroxyethyl, 2-aminoethyl, 2-(dimethylamino)ethyl, arylalkyl (also called “aralkyl”, e.g., benzyl, chloro(phenyl)methyl, 4-methylphenylmethyl, (2,4-dimethylphenyl)methyl, o-fluorophenylmethyl, 2-phenylpropyl, 2-, 3-, or 4-carboxyphenylalkyl), or heteroarylalkyl (also called “heteroaralkyl”).

The term “alkylene” refers to a diradical of a saturated straight or branched hydrocarbon. Preferably, the alkylene comprises from 1 to 12 (such as 1 to 10) carbon atoms, i.e., 1, 2, 3, 4, 5, 6, 7, 8, 9, 10, 11, or 12 carbon atoms (such as 1, 2, 3, 4, 5, 6, 7, 8, 9, or 10 carbon atoms), more preferably 1 to 8 carbon atoms, such as 1 to 6 or 1 to 4 carbon atoms. Exemplary alkylene groups include methylene, ethylene (i.e., 1,1-ethylene, 1,2-ethylene), propylene (i.e., 1,1-propylene, 1,2-propylene (—CH(CH₃)CH₂—), 2,2-propylene (—C(CH₃)₂—), and 1,3-propylene), the butylene isomers (e.g., 1,1-butylene, 1,2-butylene, 2,2-butylene, 1,3-butylene, 2,3-butylene (cis or trans or a mixture thereof), 1,4-butylene, 1,1-iso-butylene, 1,2-iso-butylene, and 1,3-iso-butylene), the pentylene isomers (e.g., 1,1-pentylene, 1,2-pentylene, 1,3-pentylene, 1,4-pentylene, 1,5-pentylene, 1,1-iso-pentylene, 1,1-sec-pentyl, 1,1-neo-pentyl), the hexylene isomers (e.g., 1,1-hexylene, 1,2-hexylene, 1,3-hexylene, 1,4-hexylene, 1,5-hexylene, 1,6-hexylene, and 1,1-isohexylene), the heptylene isomers (e.g., 1,1-heptylene, 1,2-heptylene, 1,3-heptylene, 1,4-heptylene, 1,5-heptylene, 1,6-heptylene, 1,7-heptylene, and 1,1-isoheptylene), the octylene isomers (e.g., 1,1-octylene, 1,2-octylene, 1,3-octylene, 1,4-octylene, 1,5-octylene, 1,6-octylene, 1,7-octylene, 1,8-octylene, and 1,1-isooctylene), and the like. The straight alkylene moieties having at least 3 carbon atoms and a free valence at each end can also be designated as a multiple of methylene (e.g., 1,4-butylene can also be called tetramethylene). Generally, instead of using the ending “ylene” for alkylene moieties as specified above, one can also use the ending “diyl” (e.g., 1,2-butylene can also be called butan-1,2-diyl). A “substituted alkylene” means that one or more (such as 1 to the maximum number of hydrogen atoms bound to an alkylene group, e.g., 1, 2, 3, 4, 5, 6, 7, 8, 9, or up to 10, such as between 1 to 5, 1 to 4, or 1 to 3, or 1 or 2) hydrogen atoms of the alkylene group are replaced with a substituent other than hydrogen (when more than one hydrogen atom is replaced the substituents may be the same or different). Preferably, the substituent other than hydrogen is a 1^{st} level substituent, a 2^{nd} level substituent, or a 3^{rd} level substituent as specified herein, such as halogen or optionally substituted aryl. Examples of a substituted alkylene include chloromethylene, dichloromethylene, fluoromethylene, and difluoromethylene.

The term “alkenyl” refers to a monoradical of an unsaturated straight or branched hydrocarbon having at least one carbon-carbon double bond. Generally, the maximal number of carbon-carbon double bonds in the alkenyl group can be equal to the integer which is calculated by dividing the number of carbon atoms in the alkenyl group by 2 and, if the number of carbon atoms in the alkenyl group is uneven, rounding the result of the division down to the next integer. For example, for an alkenyl group having 9 carbon atoms, the maximum number of carbon-carbon double bonds is 4. Preferably, the alkenyl group has 1 to 6 (such as 1 to 4), i.e., 1, 2, 3, 4, 5, or 6, carbon-carbon double bonds. Preferably, the alkenyl group comprises from 2 to 12 (such as 2 to 10) carbon atoms, i.e., 2, 3, 4, 5, 6, 7, 8, 9, 10, 11, or 12 carbon atoms (such as 2, 3, 4, 5, 6, 7, 8, 9, or 10 carbon atoms), more preferably 2 to 8 carbon atoms, such as 2 to 6 carbon atoms or 2 to 4 carbon atoms. Thus, in a preferred embodiment, the alkenyl group comprises from 2 to 12 (e.g., 2 to 10) carbon atoms and 1, 2, 3, 4, 5, or 6 (e.g., 1, 2, 3, 4, or 5) carbon-carbon double bonds, more preferably it comprises 2 to 8 carbon atoms and 1, 2, 3, or 4 carbon-carbon double bonds, such as 2 to 6 carbon atoms and 1, 2, or 3 carbon-carbon double bonds or 2 to 4 carbon atoms and 1 or 2 carbon-carbon double bonds. The carbon-carbon double bond(s) may be in cis (Z) or trans (E) configuration. Exemplary alkenyl groups include vinyl, 1-propenyl, 2-propenyl (i.e., allyl), 1-butenyl, 2-butenyl, 3-butenyl, 1-pentenyl, 2-pentenyl, 3-pentenyl, 4-pentenyl, 1-hexenyl, 2-hexenyl, 3-hexenyl, 4-hexenyl, 5-hexenyl, 1-heptenyl, 2-heptenyl, 3-heptenyl, 4-heptenyl, 5-heptenyl, 6-heptenyl, 1-octenyl, 2-octenyl, 3-octenyl, 4-octenyl, 5-octenyl, 6-octenyl, 7-octenyl, 1-nonenyl, 2-nonenyl, 3-nonenyl, 4-nonenyl, 5-nonenyl, 6-nonenyl, 7-nonenyl, 8-nonenyl, 1-decenyl, 2-decenyl, 3-decenyl, 4-decenyl, 5-decenyl, 6-decenyl, 7-decenyl, 8-decenyl, 9-decenyl, 1-undecenyl, 2-undecenyl, 3-undecenyl, 4-undecenyl, 5-undecenyl, 6-undecenyl, 7-undecenyl, 8-undecenyl, 9-undecenyl, 10-undecenyl, 1-dodecenyl, 2-dodecenyl, 3-dodecenyl, 4-dodecenyl, 5-dodecenyl, 6-dodecenyl, 7-dodecenyl, 8-dodecenyl, 9-dodecenyl, 10-dodecenyl, 11-dodecenyl, and the like. If an alkenyl group is attached to a nitrogen atom, the double bond cannot be alpha to the nitrogen atom. A “substituted alkenyl” means that one or more (such as 1 to the maximum number of hydrogen atoms bound to an alkenyl group, e.g., 1, 2, 3, 4, 5, 6, 7, 8, 9, or up to 10, such as between 1 to 5, 1 to 4, or 1 to 3, or 1 or 2) hydrogen atoms of the alkenyl group are replaced with a substituent other than hydrogen (when more than one hydrogen atom is replaced the substituents may be the same or different). Preferably, the substituent other than hydrogen is a 1^{st} level substituent, a 2^{nd} level substituent, or a 3^{rd} level substituent as specified herein, such as halogen or optionally substituted aryl. An example of a substituted alkenyl is styryl (i.e., 2-phenylvinyl).

The term “alkenylene” refers to a diradical of an unsaturated straight or branched hydrocarbon having at least one carbon-carbon double bond. Generally, the maximal number of carbon-carbon double bonds in the alkenylene group can be equal to the integer which is calculated by dividing the number of carbon atoms in the alkenylene group by 2 and, if the number of carbon atoms in the alkenylene group is uneven, rounding the result of the division down to the next integer. For example, for an alkenylene group having 9 carbon atoms, the maximum number of carbon-carbon double bonds is 4. Preferably, the alkenylene group has 1 to 6 (such as 1 to 4), i.e., 1, 2, 3, 4, 5, or 6, carbon-carbon double bonds. Preferably, the alkenylene group comprises from 2 to 12 (such as 2 to 10) carbon atoms, i.e., 2, 3, 4, 5, 6, 7, 8, 9, 10, 11, or 12 carbon atoms (such as 2, 3, 4, 5, 6, 7, 8, 9, or 10 carbon atoms), more preferably 2 to 8 carbon atoms, such as 2 to 6 carbon atoms or 2 to 4 carbon atoms. Thus, in a preferred embodiment, the alkenylene group comprises from 2 to 12 (such as 2 to 10 carbon) atoms and 1, 2, 3, 4, 5, or 6 (such as 1, 2, 3, 4, or 5) carbon-carbon double bonds, more preferably it comprises 2 to 8 carbon atoms and 1, 2, 3, or 4 carbon-carbon double bonds, such as 2 to 6 carbon atoms and 1, 2, or 3 carbon-carbon double bonds or 2 to 4 carbon atoms and 1 or 2 carbon-carbon double bonds. The carbon-carbon double bond(s) may be in cis (Z) or trans (E) configuration. Exemplary alkenylene groups include ethen-1,2-diyl, vinylidene (also called ethenylidene), 1-propen-1,2-diyl, 1-propen-1,3-diyl, 1-propen-2,3-diyl, allylidene, 1-buten-1,2-diyl, 1-buten-1,3-diyl, 1-buten-1,4-diyl, 1-buten-2,3-diyl, 1-buten-2,4-diyl, 1-buten-3,4-diyl, 2-buten-1,2-diyl, 2-buten-1,3-diyl, 2-buten-1,4-diyl, 2-buten-2,3-diyl, 2-buten-2,4-diyl, 2-buten-3,4-diyl, and the like. If an alkenylene group is attached to a nitrogen atom, the double bond cannot be alpha to the nitrogen atom. A “substituted alkenylene” means that one or more (such as 1 to the maximum number of hydrogen atoms bound to an alkenylene group, e.g., 1, 2, 3, 4, 5, 6, 7, 8, 9, or up to 10, such as between 1 to 5, 1 to 4, or 1 to 3, or 1 or 2) hydrogen atoms of the alkenylene group are replaced with a substituent other than hydrogen (when more than one hydrogen atom is replaced the substituents may be the same or different). Preferably, the substituent other than hydrogen is a 1^{st} level substituent, a 2^{nd} level substituent, or a 3^{rd} level substituent as specified herein, such as halogen or optionally substituted aryl. Examples of a substituted alkenylene are 1-phenyl-ethen-1,2-diyl and 2-phenyl-ethen-1,2-diyl.

The term “alkynyl” refers to a monoradical of an unsaturated straight or branched hydrocarbon having at least one carbon-carbon triple bond. Generally, the maximal number of carbon-carbon triple bonds in the alkynyl group can be equal to the integer which is calculated by dividing the number of carbon atoms in the alkynyl group by 2 and, if the number of carbon atoms in the alkynyl group is uneven, rounding the result of the division down to the next integer. For example, for an alkynyl group having 9 carbon atoms, the maximum number of carbon-carbon triple bonds is 4. Preferably, the alkynyl group has 1 to 6 (such as 1 to 4), i.e., 1, 2, 3, 4, 5, or 6, more preferably 1 or 2 carbon-carbon triple bonds. Preferably, the alkynyl group comprises from 2 to 12 (such as 2 to 10) carbon atoms (such as 1, 2, 3, 4, 5, 6, 7, 8, 9, or 10 carbon atoms), i.e., 2, 3, 4, 5, 6, 7, 8, 9, 10, 11, or 12 carbon atoms, more preferably 2 to 8 carbon atoms, such as 2 to 6 carbon atoms or 2 to 4 carbon atoms. Thus, in a preferred embodiment, the alkynyl group comprises from 2 to 12 (such as 2 to 10) carbon atoms and 1, 2, 3, 4, 5, or 6 (such as 1, 2, 3, 4, or 5 (preferably 1, 2, or 3)) carbon-carbon triple bonds, more preferably it comprises 2 to 8 carbon atoms and 1, 2, 3, or 4 (preferably 1 or 2) carbon-carbon triple bonds, such as 2 to 6 carbon atoms and 1, 2 or 3 carbon-carbon triple bonds or 2 to 4 carbon atoms and 1 or 2 carbon-carbon triple bonds. Exemplary alkynyl groups include ethynyl, 1-propynyl, 2-propynyl, 1-butynyl, 2-butynyl, 3-butynyl, 1-pentynyl, 2-pentynyl, 3-pentynyl, 4-pentynyl, 1-hexynyl, 2-hexynyl, 3-hexynyl, 4-hexynyl, 5-hexynyl, 1-heptynyl, 2-heptynyl, 3-heptynyl, 4-heptynyl, 5-heptynyl, 6-heptynyl, 1-octynyl, 2-octynyl, 3-octynyl, 4-octynyl, 5-octynyl, 6-octynyl, 7-octynyl, 1-nonylyl, 2-nonynyl, 3-nonynyl, 4-nonynyl, 5-nonynyl, 6-nonynyl, 7-nonynyl, 8-nonynyl, 1-decynyl, 2-decynyl, 3-decynyl, 4-decynyl, 5-decynyl, 6-decynyl, 7-decynyl, 8-decynyl, 9-decynyl, and the like. If an alkynyl group is attached to a nitrogen atom, the triple bond cannot be alpha to the nitrogen atom. A “substituted alkynyl” means that one or more (such as 1 to the maximum number of hydrogen atoms bound to an alkynyl group, e.g., 1, 2, 3, 4, 5, 6, 7, 8, 9, or up to 10, such as between 1 to 5, 1 to 4, or 1 to 3, or 1 or 2) hydrogen atoms of the alkynyl group are replaced with a substituent other than hydrogen (when more than one hydrogen atom is replaced the substituents may be the same or different). Preferably, the substituent other than hydrogen is a 1^{st} level substituent, a 2^{nd} level substituent, or a 3^{rd} level substituent as specified herein, such as halogen or optionally substituted aryl.

The term “alkynylene” refers to a diradical of an unsaturated straight or branched hydrocarbon having at least one carbon-carbon triple bond. Generally, the maximal number of carbon-carbon triple bonds in the alkynylene group can be equal to the integer which is calculated by dividing the number of carbon atoms in the alkynylene group by 2 and, if the number of carbon atoms in the alkynylene group is uneven, rounding the result of the division down to the next integer. For example, for an alkynylene group having 9 carbon atoms, the maximum number of carbon-carbon triple bonds is 4. Preferably, the alkynylene group has 1 to 6 (such as 1 to 4), i.e., 1, 2, 3, 4, 5, or 6 (such as 1, 2, 3, or 4), more preferably 1 or 2 carbon-carbon triple bonds. Preferably, the alkynylene group comprises from 2 to 12 (such as 2 to 10) carbon atoms, i.e., 2, 3, 4, 5, 6, 7, 8, 9, 10, 11, or 12 carbon atoms (such as 2, 3, 4, 5, 6, 7, 8, 9, or 10 carbon atoms), more preferably 2 to 8 carbon atoms, such as 2 to 6 carbon atoms or 2 to 4 carbon atoms. Thus, in a preferred embodiment, the alkynylene group comprises from 2 to 12 (such as 2 to 10) carbon atoms and 1, 2, 3, 4, 5, or 6 (such as 1, 2, 3, 4, or 5 (preferably 1, 2, or 3)) carbon-carbon triple bonds, more preferably it comprises 2 to 8 carbon atoms and 1, 2, 3, or 4 (preferably 1 or 2) carbon-carbon triple bonds, such as 2 to 6 carbon atoms and 1, 2 or 3 carbon-carbon triple bonds or 2 to 4 carbon atoms and 1 or 2 carbon-carbon triple bonds. Exemplary alkynylene groups include ethyn-1,2-diyl, 1-propyn-1,3-diyl, 1-propyn-3,3-diyl, 1-butyn-1,3-diyl, 1-butyn-1,4-diyl, 1-butyn-3,4-diyl, 2-butyn-1,4-diyl and the like. If an alkynylene group is attached to a nitrogen atom, the triple bond cannot be alpha to the nitrogen atom. A “substituted alkynylene” means that one or more (such as 1 to the maximum number of hydrogen atoms bound to an alkynylene group, e.g., 1, 2, 3, 4, 5, 6, 7, 8, 9, or up to 10, such as between 1 to 5, 1 to 4, or 1 to 3, or 1 or 2) hydrogen atoms of the alkynylene group are replaced with a substituent other than hydrogen (when more than one hydrogen atom is replaced the substituents may be the same or different). Preferably, the substituent other than hydrogen is a 1^{st} level substituent, a 2^{nd} level substituent, or a 3^{rd} level substituent as specified herein, such as halogen or optionally substituted aryl.

The term “aryl” or “aromatic ring” refers to a monoradical of an aromatic cyclic hydrocarbon. Preferably, the aryl group contains 3 to 14 (e.g., 5, 6, 7, 8, 9, or 10, such as 5, 6, or 10) carbon atoms which can be arranged in one ring (e.g., phenyl) or two or more condensed rings (e.g., naphthyl). Exemplary aryl groups include cyclopropenylium, cyclopentadienyl, phenyl, indenyl, naphthyl, azulenyl, fluorenyl, anthryl, and phenanthryl. Preferably, “aryl” refers to a monocyclic ring containing 6 carbon atoms or an aromatic bicyclic ring system containing 10 carbon atoms. Preferred examples are phenyl and naphthyl. Aryl does not encompass fullerenes. A “substituted aryl” means that one or more (such as 1 to the maximum number of hydrogen atoms bound to an aryl group, e.g., 1, 2, 3, 4, 5, 6, 7, 8, 9, or up to 10, such as between 1 to 5, 1 to 4, or 1 to 3, or 1 or 2) hydrogen atoms of the aryl group are replaced with a substituent other than hydrogen (when more than one hydrogen atom is replaced the substituents may be the same or different). Preferably, the substituent other than hydrogen is a 1^{st} level substituent, a 2^{nd} level substituent, or a 3^{rd} level substituent as specified herein, such as halogen, —CN, nitro, —OR¹¹ (e.g., —OH), —SR¹¹ (e.g., —SH), —N(R¹²)(R¹³) (e.g., —NH₂), alkyl (e.g., C₁₋₆ alkyl), alkenyl (e.g., C₂₋₆ alkenyl), and alkynyl (e.g., C₂₋₆ alkynyl). Examples of a substituted aryl include biphenyl, 2-fluorophenyl, 2-chloro-6-methylphenyl, anilinyl, 3-nitrophenyl, 4-hydroxyphenyl, methoxyphenyl (i.e., 2-, 3-, or 4-methoxyphenyl), and 4-ethoxyphenyl.

The term “heteroaryl” or “heteroaromatic ring” means an aryl group as defined above in which one or more carbon atoms in the aryl group are replaced by heteroatoms (such as O, S, or N). Preferably, heteroaryl refers to a five or six-membered aromatic monocyclic ring, wherein 1, 2, or 3 carbon atoms are replaced by the same or different heteroatoms of O, N, or S. Alternatively, it means an aromatic bicyclic or tricyclic ring system wherein 1, 2, 3, 4, or 5 carbon atoms are replaced with the same or different heteroatoms of O, N, or S. Preferably, in each ring of the heteroaryl group the maximum number of o atoms is 1, the maximum number of S atoms is 1, and the maximum total number of O and S atoms is 2. For example, 3- to 14-membered heteroaryl encompasses monocyclic heteroaryl (e.g., 5- or 6-membered), bicyclic heteroaryl (e.g., 9- or 10-membered), and tricyclic heteroaryl (e.g., 13- or 14-membered). Exemplary heteroaryl groups include furanyl, thienyl, oxazolyl, isoxazolyl, oxadiazolyl (1,2,5- and 1,2,3-), pyrrolyl, imidazolyl, pyrazolyl, triazolyl (1,2,3- and 1,2,4-), tetrazolyl, thiazolyl, isothiazolyl, thiadiazolyl (1,2,3- and 1,2,5-), pyridyl (also called pyridinyl), pyrimidinyl, pyrazinyl, triazinyl (1,2,3-, 1,2,4-, and 1,3,5-), benzofuranyl (1- and 2), indolyl, isoindolyl, benzothienyl (1- and 2-), 1H-indazolyl, benzimidazolyl, benzoxazolyl, indoxazinyl, benzisoxazolyl, benzothiazolyl, benzisothiazolyl, benzotriazolyl, quinolinyl, isoquinolinyl, benzodiazinyl, quinoxalinyl, quinazolinyl, benzotriazinyl (1,2,3- and 1,2,4-benzotriazinyl), pyridazinyl, phenoxazinyl, thiazolopyridinyl, pyrrolothiazolyl, phenothiazinyl, isobenzofuranyl, chromenyl, xanthenyl, phenoxathiinyl, pyrrolizinyl, indolizinyl, indazolyl, purinyl, quinolizinyl, phthalazinyl, naphthyridinyl (1,5-, 1,6-, 1,7-, 1,8-, and 2,6-), cinnolinyl, pteridinyl, carbazolyl, phenanthridinyl, acridinyl, perimidinyl, phenanthrolinyl (1,7-, 1,8-, 1,10-, 3,8-, and 4,7-), phenazinyl, oxazolopyridinyl, isoxazolopyridinyl, pyrrolooxazolyl, and pyrrolopyrrolyl. Exemplary 5- or 6-membered heteroaryl groups include furanyl, thienyl, oxazolyl, isoxazolyl, oxadiazolyl (1,2,5- and 1,2,3-), pyrrolyl, imidazolyl, pyrazolyl, triazolyl (1,2,3- and 1,2,4-), thiazolyl, isothiazolyl, thiadiazolyl (1,2,3- and 1,2,5), pyridyl, pyrimidinyl, pyrazinyl, triazinyl (1,2,3-, 1,2,4-, and 1,3,5-), and pyridazinyl. A “substituted heteroaryl” means that one or more (such as 1 to the maximum number of hydrogen atoms bound to a heteroaryl group, e.g., 1, 2, 3, 4, 5, 6, 7, 8, 9, or up to 10, such as between 1 to 5, 1 to 4, or 1 to 3, or 1 or 2) hydrogen atoms of the heteroaryl group are replaced with a substituent other than hydrogen (when more than one hydrogen atom is replaced the substituents may be the same or different). Preferably, the substituent other than hydrogen is a 1^{st} level substituent, a 2^{nd} level substituent, or a 3^{rd} level substituent as specified herein, such as halogen, CN, nitro, —OR¹¹ (e.g., —OH), —SR¹¹ (e.g., —SH), —N(R¹²)(R¹³) (e.g., —NH₂), alkyl (e.g., C₁₋₆ alkyl), alkenyl (e.g., C₂₋₆ alkenyl), and alkynyl (e.g., C₂₋₆ alkynyl). Examples of a substituted heteroaryl include 2,4-dimethylpyridin-3-yl, 2-methyl-4-bromopyridin-3-yl, 3-methyl-2-pyridin-2-yl, 3-chloro-5-methylpyridin-4-yl, 4-chloro-2-methylpyridin-3-yl, 3,5-dimethylpyridin-4-yl, 2-methylpyridin-3-yl, 2-chloro-4-methyl-thien-3-yl, 1,3,5-trimethylpyrazol-4-yl, 3,5-dimethyl-1,2-dioxazol-4-yl, 1,2,4-trimethylpyrrol-3-yl, 3-phenylpyrrolyl, 2,3′-bifuryl, 4-methylpyridyl, 2-, or 3-ethylindolyl.

The term “cycloalkyl” or “cycloaliphatic” represents cyclic non-aromatic versions of “alkyl” and “alkenyl” with preferably 3 to 14 carbon atoms, such as 3 to 12 or 3 to 10 carbon atoms, i.e., 3, 4, 5, 6, 7, 8, 9, 10, 11, 12, 13, or 14 carbon atoms (such as 3, 4, 5, 6, 7, 8, 9, or 10 carbon atoms), more preferably 3 to 7 carbon atoms. Exemplary cycloalkyl groups include cyclopropyl, cyclopropenyl, cyclobutyl, cyclobutenyl, cyclopentyl, cyclopentenyl, cyclohexyl, cyclohexenyl, cycloheptyl, cycloheptenyl, cyclooctyl, cyclooctenyl, cyclononyl, cyclononenyl, cylcodecyl, cylcodecenyl, and adamantyl. The term “cycloalkyl” is also meant to include bicyclic and tricyclic versions thereof. If bicyclic rings are formed it is preferred that the respective rings are connected to each other at two adjacent carbon atoms, however, alternatively the two rings are connected via the same carbon atom, i.e., they form a spiro ring system or they form “bridged” ring systems. Preferred examples of cycloalkyl include C₃-8-cycloalkyl, in particular cyclopropyl, cyclobutyl, cyclopentyl, cyclohexyl, cycloheptyl, cyclooctyl, spiro[3,3]heptyl, spiro[3,4]octyl, spiro[4,3]octyl, bicyclo[4.1.0]heptyl, bicyclo[3.2.0]heptyl, bicyclo[2.2.1]heptyl, bicyclo[2.2.2]octyl, bicyclo[5.1.0]octyl, and bicyclo[4.2.0]octyl. Cycloalkyl does not encompass fullerenes. A “substituted cycloalkyl” means that one or more (such as 1 to the maximum number of hydrogen atoms bound to a cycloalkyl group, e.g., 1, 2, 3, 4, 5, 6, 7, 8, 9, or up to 10, such as between 1 to 5, 1 to 4, or 1 to 3, or 1 or 2) hydrogen atoms of the cycloalkyl group are replaced with a substituent other than hydrogen (when more than one hydrogen atom is replaced the substituents may be the same or different). Preferably, the substituent other than hydrogen is a 1^{st} level substituent, a 2^{nd} level substituent, or a 3^{rd} level substituent as specified herein, such as halogen, —CN, nitro, —OR¹¹ (e.g., —OH), —SR¹¹ (e.g., —SH), —N(R¹²)(R¹³) (e.g., —NH₂), ═X (e.g., ═O, ═S, or ═NH), alkyl (e.g., C₁₋₆ alkyl), alkenyl (e.g., C₂₋₆ alkenyl), and alkynyl (e.g., C₂₋₆ alkynyl). Examples of a substituted cycloalkyl include oxocyclohexyl, oxocyclopentyl, fluorocyclohexyl, and oxocyclohexenyl.

The expression “heterocyclyl comprising at least one O as the only heteroatom element” as used herein (and similar expressions) means that the subject heterocyclyl moiety contains only the elements carbon and oxygen as ring atoms. Preferably, such subject heterocyclyl moieties contain only one oxygen atom as a ring atom, although other such subject heterocyclyl moieties includes those with two or more oxygen atoms as a ring, typically wherein neighboring oxygen ring atoms are separated by at least one carbon ring atom.

The term “heterocyclyl” or “heterocyclic ring” means a cycloalkyl group as defined above in which from 1, 2, 3, or 4 ring carbon atoms in the cycloalkyl group are replaced by heteroatoms (such as those selected from the group consisting of O, S, S(O), S(O)₂, N, B, Si, and P, preferably selected from the group consisting of O, S, S(O)₂, and N, more preferably selected from the group consisting of O, S, and N). If a ring of the heterocyclyl group only contains one type of heteroatom, the maximum number of said heteroatom in the ring of said heterocyclyl group may be as follows: 2 O atoms (preferably 1 O atom); 2 S atoms (preferably 1 S atom); 4 N atoms (such as 1, 2, or 3 N atoms); 2 B atoms (preferably 1 B atom); 1 Si atom; and/or 1 P atom. If a ring of the heterocyclyl group contains two or more types of heteroatoms, the maximum number of said heteroatoms in the ring of said heterocyclyl group may be as follows: 1 O atom; 1 S atom; 2 N atoms (preferably 1 N atom); 1 B atom; 1 Si atom; and/or 1 P atom, wherein the maximum total number of heteroatoms in the ring of said heterocyclyl group is 4 and the maximum total number of each heteroatom in the ring of said heterocyclyl group is as follows: 1 O atom; 1 S atom; 1 or 2 N atoms; 1 B atom (preferably 0 B atom); 1 Si atom (preferably 0 Si atom); and/or 1 P atom (preferably 0 P atom). In one embodiment, the heteroatoms of the heterocyclyl group are selected from the group consisting of O, S, and N. In this embodiment, preferably, in each ring of the heterocyclyl group the maximum number of O atoms is 1, the maximum number of S atoms is 1, and the maximum total number of O and S atoms is 2. For example, 3- to 14-membered heterocyclyl encompasses monocyclic heterocyclyl (e.g., 3-, 4-, 5-, 6-, or 7-membered, preferably 4- to 7-membered), bicyclic heterocyclyl (e.g., 8-, 9-, or 10-membered), and tricyclic heterocyclyl (e.g., 12-, 13-, or 14-membered). If a heterocyclyl group comprises two or more rings, these rings either are fused (such as in quinolinyl or purinyl), are a spiro moiety, are a bridged structure, are linked via a double bond, or are a combination thereof. In other words, an unsubstituted heterocyclyl group does not encompass two heterocyclyl groups linked via a single bond. The term “heterocyclyl” is also meant to encompass partially or completely hydrogenated forms (such as dihydro, tetrahydro, hexahydro, octahydro, decahydro, dodecahydro, etc., or perhydro forms) of the above-mentioned heteroaryl groups. Exemplary heterocyclyl groups include azetidinyl, morpholino, isochromanyl, chromanyl, pyrrolidinyl, imidazolidinyl, pyrazolidinyl, piperidinyl, piperazinyl, indolinyl, isoindolinyl, triazininanyl (1,2,3-, 1,2,4-, and 1,3,5-), di- and tetrahydrofuranyl, di- and tetrahydrothienyl, di- and tetrahydrooxazolyl, di- and tetrahydroisoxazolyl, di- and tetrahydrooxadiazolyl (1,2,5- and 1,2,3-), dihydropyrrolyl, dihydroimidazolyl, dihydropyrazolyl, di- and tetrahydrotriazolyl (1,2,3- and 1,2,4-), di- and tetrahydrothiazolyl, di- and tetrahydrothiazolyl, di- and tetrahydrothiadiazolyl (1,2,3- and 1,2,5-), di- and tetrahydropyridyl, di-, tetra- and hexahydropyrimidinyl, di- and tetrahydropyrazinyl, di- and tetrahydrotriazinyl (1,2,3-, 1,2,4-, and 1,3,5-), di-, tetra-, hexa- and octahydrobenzofuranyl (1- and 2-), di-, tetra-, hexa- and octahydroindolyl, di-, tetra-, hexa- and octahydroisoindolyl, di-, tetra-, hexa- and octahydrobenzothienyl (1- and 2), di-, tetra-, hexa- and octahydro-1H-indazolyl, di-, tetra-, hexa- and octahydrobenzimidazolyl, di-, tetra-, hexa- and octahydrobenzoxazolyl, di-, tetra-, hexa- and octahydroindoxazinyl, di-, tetra-, hexa- and octahydrobenzisoxazolyl, di-, tetra-, hexa- and octahydrobenzothiazolyl, di-, tetra-, hexa- and octahydrobenzisothiazolyl, di-, tetra-, hexa- and octahydrobenzotriazolyl, di-, tetra-, hexa-, octa- and decahydroquinolinyl, di-, tetra-, hexa-, octa- and decahydroisoquinolinyl, di-, tetra-, hexa-, octa- and decahydrobenzodiazinyl, di-, tetra-, hexa-, octa- and decahydroquinoxalinyl, di-, tetra-, hexa-, octa- and decahydroquinazolinyl, di-, tetra-, hexa-, octa- and decahydrobenzotriazinyl (1,2,3- and 1,2,4-), di-, tetra-, and hexahydropyridazinyl, di-, tetra-, hexa-, octa-, deca- and dodecahydrophenoxazinyl, di-, tetra-, hexa-, and octahydrothiazolopyridinyl (such as 4,5,6-7-tetrahydro[1,3]thiazolo[5,4-c]pyridinyl or 4,5,6-7-tetrahydro[1,3]thiazolo[4,5-c]pyridinyl, e.g., 4,5,6-7-tetrahydro[1,3]-thiazolo[5,4-c]pyridin-2-yl or 4,5,6-7-tetrahydro[1,3]thiazolo[4,5-c]pyridin-2-yl), di-, tetra-, and hexahydro-pyrrolothiazolyl, di-, tetra-, hexa-, octa- and decahydrophenothiazinyl, di-, tetra-, hexa-, and octahydroisobenzofuranyl, di-, tetra-, hexa-, and octahydrochromenyl, di-, tetra-, hexa-, octa-, deca-, and dodecahydroxanthenyl, di-, tetra-, hexa-, octa-, deca-, and dodecahydrophenoxathiinyl, di-, tetra-, and hexahydropyrrolizinyl, di-, tetra-, hexa-, and octahydroindolizinyl, di-, tetra-, hexa-, and octahydroindazolyl, di-, tetra-, hexa-, and octahydropurinyl, di-, tetra-, hexa-, and octahydroquinolizinyl, di-, tetra-, hexa-, octa- and decahydrophthalazinyl, di-, tetra-, hexa-, octa- and decahydronaphthyridinyl (1,5-, 1,6-, 1,7-, 1,8-, and 2,6-), di-, tetra-, hexa-, octa- and decahydrocinnolinyl, di-, tetra-, hexa-, octa-, and decahydropteridinyl, di-, tetra-, hexa-, octa-, deca- and dodecahydrocarbazolyl, di-, tetra-, hexa-, octa-, deca-, dodeca-, and tetradecahydrophenanthridinyl, di-, tetra-, hexa-, octa-, deca-, dodeca-, and tetradecahydroacridinyl, di-, tetra-, hexa-, octa-, deca- and dodecahydroperimidinyl, di-, tetra-, hexa-, octa-, deca-, dodeca-, and tetradecahydrophenanthrolinyl (1,7-, 1,8-, 1,10-, 3,8-, and 4,7-), di-, tetra-, hexa-, octa-, deca-, dodeca-, and tetradecahydrophenazinyl, di-, tetra-, hexa- and octahydrooxazolopyridinyl, di-, tetra-, hexa- and octahydroisoxazolopyridinyl, di-, tetra-, hexa- and octahydrocyclopentapyrrolyl, di-, tetra-, hexa- and octahydrocyclopentpyrazolyl, di-, tetra-, hexa- and octahydrocyclopentaimidazolyl, di-, tetra-, hexa- and octahydro-cyclopentathiazolyl, di-, tetra-, hexa- and octahydrocyclopentaoxazolyl, di-, tetra-, hexa- and octahydropyrrolopyrrolyl, di-, tetra-, hexa- and octahydropyrrolopyrazolyl, di-, tetra-, hexa- and octahydropyrroloimidazolyl, di-, tetra-, hexa- and octahydropyrrolothiazolyl (such as 5,6-dihydro-4H-pyrrolo[3,4-d][1,3]thiazolyl), di-, tetra-, hexa- and octahydropyrrolooxazolyl, di-, tetra-, hexa- and octahydropyrazolopyrazolyl, di-, tetra-, hexa- and octahydro-pyrazoloimidazolyl, di-, tetra-, hexa- and octahydropyrazolothiazolyl, di-, tetra-, hexa- and octahydropyrazolooxazolyl, di-, tetra-, hexa- and octahydroimidazoimidazolyl, di-, tetra-, hexa- and octahydroimidazothiazolyl, di-, tetra-, hexa- and octahydroimidazooxazolyl, di-, tetra-, hexa- and octahydrothiazolothiazolyl, di-, tetra-, hexa- and octahydrothiazolooxazolyl, and di-, tetra-, hexa- and octahydrooxazolooxazolyl. Exemplary 5- or 6-membered heterocyclyl groups include morpholino, pyrrolidinyl, imidazolidinyl, pyrazolidinyl, piperidinyl, piperazinyl, di- and tetrahydrofuranyl, di- and tetrahydrothienyl, di- and tetrahydrooxazolyl, di- and tetrahydroisoxazolyl, di- and tetrahydrooxadiazolyl (1,2,5- and 1,2,3-), dihydropyrrolyl, dihydroimidazolyl, dihydropyrazolyl, di- and tetrahydrotriazolyl (1,2,3- and 1,2,4-), di- and tetrahydrothiazolyl, di- and tetrahydroisothiazolyl, di- and tetrahydrothiadiazolyl (1,2,3- and 1,2,5-), di- and tetrahydropyridyl, di-, tetra-, and hexahydropyrimidinyl, di- and tetrahydropyrazinyl, di- and tetrahydrotriazinyl (1,2,3-, 1,2,4-, and 1,3,5-), and triazinanyl (1,2,3-, 1,2,4-, and 1,3,5-). A “substituted heterocyclyl” means that one or more (such as 1 to the maximum number of hydrogen atoms bound to a heterocyclyl group, e.g., 1, 2, 3, 4, 5, 6, 7, 8, 9, or up to 10, such as between 1 to 5, 1 to 4, or 1 to 3, or 1 or 2) hydrogen atoms of the heterocyclyl group are replaced with a substituent other than hydrogen (when more than one hydrogen atom is replaced the substituents may be the same or different). Preferably, the substituent other than hydrogen is a 1^{st} level substituent, a 2^{nd} level substituent, or a 3^{rd} level substituent as specified herein, such as halogen, —CN, nitro, —OR¹¹ (e.g., —OH), —SR¹¹ (e.g., —SH), —N(R¹²)(R¹³) (e.g., —NH₂), ═X (e.g., ═O, ═S, or ═NH), alkyl (e.g., C₁₋₆ alkyl), alkenyl (e.g., C₂₋₆ alkenyl), and alkynyl (e.g., C₂₋₆ alkynyl).

The expression “partially hydrogenated form” of an unsaturated compound or group as used herein means that part of the unsaturation has been removed by formally adding hydrogen to the initially unsaturated compound or group without removing all unsaturated moieties. The phrase “completely hydrogenated form” of an unsaturated compound or group is used herein interchangeably with the term “perhydro” and means that all unsaturation has been removed by formally adding hydrogen to the initially unsaturated compound or group. For example, partially hydrogenated forms of a 5-membered heteroaryl group (containing 2 double bonds in the ring, such as furan) include dihydro forms of said 5-membered heteroaryl group (such as 2,3-dihydrofuran or 2,5-dihydrofuran), whereas the tetrahydro form of said 5-membered heteroaryl group (e.g., tetrahydrofuran, i.e., THF) is a completely hydrogenated (or perhydro) form of said 5-membered heteroaryl group. Likewise, for a 6-membered heteroaryl group having 3 double bonds in the ring (such as pyridyl), partially hydrogenated forms include di- and tetrahydro forms (such as di- and tetrahydropyridyl), whereas the hexahydro form (such as piperidinyl in case of the heteroaryl pyridyl) is the completely hydrogenated (or perhydro) derivative of said 6-membered heteroaryl group. Consequently, a hexahydro form of an aryl or heteroaryl can only be considered a partially hydrogenated form according to the present invention if the aryl or heteroaryl contains at least 4 unsaturated moieties consisting of double and triple bonds between ring atoms.

The term “aromatic” as used in the context of hydrocarbons means that the whole molecule has to be aromatic. For example, if a monocyclic aryl is hydrogenated (either partially or completely) the resulting hydrogenated cyclic structure is classified as cycloalkyl for the purposes of the present invention. Likewise, if a bi- or polycyclic aryl (such as naphthyl) is hydrogenated the resulting hydrogenated bi- or polycyclic structure (such as 1,2-dihydronaphthyl) is classified as cycloalkyl for the purposes of the present invention (even if one ring, such as in 1,2-dihydronaphthyl, is still aromatic). A similar distinction is made within the present application between heteroaryl and heterocyclyl. For example, indolinyl, i.e., a dihydro variant of indolyl, is classified as heterocyclyl for the purposes of the present invention, since only one ring of the bicyclic structure is aromatic and one of the ring atoms is a heteroatom.

The term “polycyclic” as used herein means that the structure has two or more (such as 2, 3, 4, 5, 6, 7, 8, 9, or 10), preferably, 2, 3, 4, or 5, more preferably, 2, 3, or 4, rings. Therefore, according to the invention, the term “polycyclic” does not encompass monocyclic structures, wherein the structures only contain one ring. Examples of polycyclic groups are fused structures (such as naphthyl or anthryl), spiro compounds, rings that are linked via single or double bonds (such as biphenyl), and bridged structures (such as bornyl). Exemplary polycyclic structures are those aryl, heteroaryl, cycloalkyl, and heterocyclyl groups specified above which have at least two rings.

The term “halogen” or “halo” or “hal” means fluoro, chloro, bromo, or iodo.

The term “azido” means —N₃.

The term “N-oxide” means an amine oxide or amine-N-oxide which is a chemical compound containing the functional group (Rⁿ)₃N⁺—O⁻, i.e., an N—O coordinate covalent bond, wherein Rⁿ is independently selected from the group consisting of hydrogen, alkyl, alkenyl, alkynyl, aryl, heteroaryl, cycloalkyl, and heterocyclyl, wherein each of the alkyl, alkenyl, alkynyl, aryl, heteroaryl, cycloalkyl, and heterocyclyl groups is optionally substituted with one or more (such as 1 to the maximum number of hydrogen atoms bound to the alkyl, alkenyl, alkynyl, aryl, heteroaryl, cycloalkyl, or heterocyclyl group, e.g., 1, 2, 3, 4, 5, 6, 7, 8, 9, or up to 10, such as between 1 to 5, 1 to 4, or 1 to 3, or 1 or 2) independently selected R³⁰, the R³⁰ preferably being a 1^{st} level substituent, a 2^{nd} level substituent, or a 3^{rd} level substituent as specified herein.

The term “impurity” as used herein refers to any foreign material (in particular chemical substances) which may be present in a composition comprising a desired compound (e.g., a composition comprising a compound described herein, such a compound of formula (I), (Ia), (II), (III), (IV), (V), (Va), (VI), (VII), (VIIa), (VIII), (IX), (X), (XI), or (XII)). Impurities may occur naturally, may be added during the synthesis and/or purification of the desired compound, or may be generated during the synthesis and/or purification of the desired compound. Exemplary impurities include one or more starting materials, one or more solvents, one or more intermediates or reactants, one or more degradation products of any of the foregoing or of the desired compound, one or more leftovers of protecting groups after deprotection, and combinations thereof.

The expression “at least one of R⁷ is F and/or at least one of R⁷ is substituted with one or more F atoms” as used herein (and similar expressions) means that R⁶ is substituted with (i) at least one F atom and/or (ii) a moiety bearing one or more (e.g., 1 to the maximum number of hydrogen atoms bound to the moiety, e.g., 1, 2, 3, 4, 5, 6, 7, 8, 9, or up to 10, such as 1 to 5, 1 to 4, or 1 to 3, or 1 or 2) F atoms. Exemplary moieties bearing one or more F atoms include an alkyl group bearing one or more (e.g., 1 to the maximum number of hydrogen atoms bound to the alkyl group, e.g., 1, 2, 3, 4, 5, 6, 7, 8, 9, or up to 10, such as 1 to 5, 1 to 4, or 1 to 3, or 1 or 2 or 3) F atoms, such as C₁₋₃alkyl bearing one or more (e.g., 1 to the maximum number of hydrogen atoms bound to the alkyl group, e.g., 1, 2, 3, 4, 5, 6, or 7, or up to 6, such as 1 to 5, 1 to 4, or 1 to 3, or 1 or 2 or 3) F atoms, e.g., —CH₂F, —CHF₂, or —CF₃. Further exemplary moieties bearing one or more F atoms include F substituted alkoxy groups (i.e., —O(alkyl), such as —O(C₁₋₃alkyl)) or F substituted alkyl amino groups (i.e., —NH(alkyl) or —N(alkyl)₂, such as —NH(C₁₋₃alkyl) or —N(C₁₋₃alkyl)₂), wherein the alkyl (e.g., the C₁₋₃alkyl) portion of the alkoxy and monoalkyl amino groups and at least one of the alkyl (e.g., the C₁₋₃alkyl) portions of the dialkylamino groups is substituted with one or more (e.g., 1 to the maximum number of hydrogen atoms bound to the alkyl portion, e.g., 1, 2, 3, 4, 5, 6, 7, 8, 9, or up to 10 or up to 7, such as 1 to 5, 1 to 4, or 1 to 3, or 1 or 2 or 3) F atoms.

As described elsewhere herein, R⁵ is -L-R⁶, and R⁶ is heteroaryl or heterocyclyl each of which is optionally substituted with one or more independently selected R⁷. In relation thereto, the expression “any two R⁷ which are bound to the same atom of R⁶ may join together to form ═O” as used herein means that two monoradicals (i.e., R⁷) when substituting in total 2 hydrogen atoms bound to only one ring atom of R⁶ can form the diradical ═O. For example, according to the invention, R⁶ being

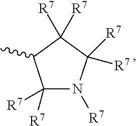

(wherein [symbol] represents the bond by which R⁶ is bound to the remainder of the compound) encompasses not only (1) the possibility that each of the R⁷ groups is a monoradical independently selected from the particular moieties specified herein (e.g., methyl or Cl) but also (2) the possibility that any two R⁷ groups bound to the same atom of R⁶ join together to form the diradical ═O resulting in a R⁶ group having the formula

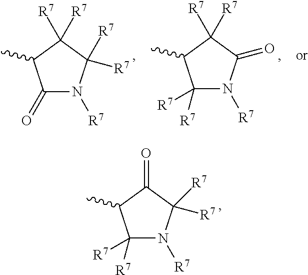

wherein the remaining R⁷ groups are monoradicals. Likewise, in case R⁶ is 3-tetrahydrothienyl substituted with four R⁷, such substituted R⁶ encompasses the following formulas:

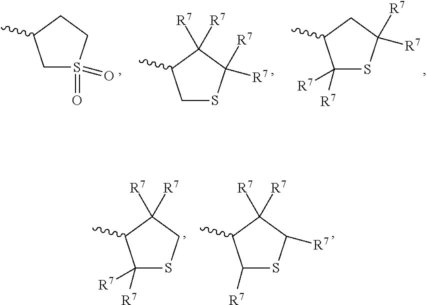

Similar terms such as “any two R³⁰ which are bound to the same carbon atom of a cycloalkyl or heterocyclyl group may join together to form ═X¹” as used herein are to be interpreted in an analogous manner. In this respect, it is to be understood that in those embodiments, where any two R⁷ which are bound to the same atom of R⁶ may join together to form ═O, R⁶ initially (i.e., without the modification ═O) has to be a heterocyclic ring (because in a heteroaromatic ring there is no carbon ring atom having two free valences).

The expression “one R⁷ group is bound to a ring atom of R⁶ at position 2 relative to the ring atom by which R⁶ is bound to the remainder of the compound” as used herein means that at least one of the two ring atoms directly adjacent to the ring atom by which R⁶ is attached to the remainder of the compound bears one R⁷ group. In other words, at least one of the ortho positions of R⁶, relative to the ring atom by which R⁶ is bound to the remainder of the compound (i.e., “yl position” of R⁶), bears a R⁷ group. For example, applying the above expression to the case where R⁶ is 3-pyridyl (thus, the yl position is the ring carbon at position 3 relative to the ring nitrogen atom) substituted with one R⁷, it follows that this R⁷ group is at position 2 or 4 of the 3-pyridyl group, as shown in the following formulas:

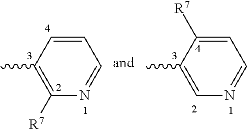

wherein [symbol] represents the bond by which R⁶ is bound to the remainder of the compound. Furthermore, in case R⁶ is substituted with more than one (such as two or three) R⁷ groups, the expression “one R⁷ group is bound to a ring atom of R⁶ at position 2 relative to the ring atom by which R⁶ is bound to the remainder of the compound” encompasses the situation that each of the two ring atoms directly adjacent to the ring atom by which R⁶ is attached to the remainder of the compound bears one R⁷ group (i.e., R⁶ being an k-membered ring bears one R⁷ group at each of positions 2 and k, relative to the ring atom by which R⁶ is bound to the remainder of the compound, i.e., R⁶ is substituted at both of its ortho positions). For example, in case R⁶ is 3-pyrrolyl (thus, the yl position is the ring carbon at position 3 relative to the ring nitrogen atom) substituted with two R⁷ groups, the expression “one R⁷ group is bound to a ring atom of R⁶ at position 2 relative to the ring atom by which R⁶ is bound to the remainder of the compound” encompasses the following structures:

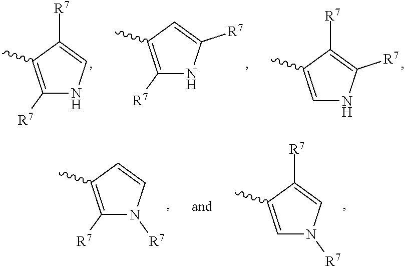

but excludes the following structure:

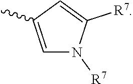

The term “k-membered ring” as used herein means that the ring has k ring atoms. E.g., for pyrazolyl k is 5; thus, relative to the ring atom (yl position) by which the pyrazolyl group is bound to the remainder of the compound, the ortho positions are positions 2 and 5 and meta positions are positions 3 and 4. Furthermore, for pyridinyl being a 6-membered heteroaryl, the ortho positions are positions 2 and 6, meta-positions are positions 3 and 5 and para-position is position 4, relative to the ring atom (yl position) by which the pyridinyl group is bound to the remainder of the compound. In case of a bicyclic ring system, such as naphthalene, ortho- and meta-positions are the positions of atoms of the same ring relative to each other. For example, in the following formula

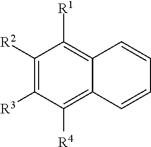

R² is in ortho-position to R¹, R³ is in meta-position to R¹, R⁴ is in para-position to R¹, R³ is in ortho-position to R², R2 is in meta-position to R⁴, etc.

Regarding R⁶ being a 5-membered monocyclic heteroaryl which contains at least one S ring atom, the expression “one R⁷ is attached to the C ring atom at position 2 relative to the ring atom by which R⁶ is bound to the remainder of the compound” as used herein (and similar expressions) preferably means that one R⁷ group is bound to the C ring atom of R⁶ which (i) is directly adjacent to the ring atom by which R⁶ is attached to the remainder of the compound and (ii) receives the lower number when numbering the ring atoms of R⁶ (e.g., starting with number “1” for the S ring atom and continuing in such a way that the number of the ring atom by which R⁶ is bound to the remainder of the compound (i.e., the “yl” position of R⁶) is as low as possible). In other words, relative to the yl position of R⁶, that C ring atom of the two “ortho” positions of R⁶ preferably bears a R⁷ group which lies between the S ring atom and the yl position of R⁶ when considering the shortest path between the S ring atom and the yl position. For example, applying the above expression to the case where R⁶ is 3-thienyl (thus, the yl position is the ring carbon at position 3 relative to the S ring atom) substituted with one R⁷, it follows that this R⁷ group is at position 2 of the 3-thienyl group, as shown in the following formula:

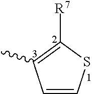

wherein [symbol] represents the bond by which R⁶ is bound to the remainder of the compound. Likewise, regarding R⁶ being a 5-membered monocyclic heteroaryl which contains at least one S ring atom, the expression “one R⁷ is attached to the C ring atom at position 5 relative to the ring atom by which R⁶ is bound to the remainder of the compound” as used herein (and similar expressions) preferably means that one R⁷ group is bound to the C ring atom of R⁶ which (i) is directly adjacent to the ring atom by which R⁶ is attached to the remainder of the compound and (ii) receives the higher number when numbering the ring atoms of R⁶ (e.g., starting with number “1” for the S ring atom and continuing in such a way that the number of the yl position of R⁶ is as low as possible). In other words, relative to the yl position of R⁶, that C ring atom preferably bears a R⁷ group which does not lie between the S ring atom and the yl position of R⁶ when considering the shortest path between the S ring atom and the yl position. For example, applying the above expression “one R⁷ group is attached to the C ring atom at position 5 relative to the ring atom by which R⁶ is bound to the remainder of the compound”) to the case where R⁶ is 3-thienyl (thus, the yl position is the ring carbon at position 3 relative to the S ring atom) substituted with one R⁷, it follows that this R⁷ group is at position 4 of the 3-thienyl group, as shown in the following formula:

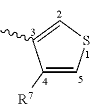

wherein [symbol] represents the bond by which R⁶ is bound to the remainder of the compound. Furthermore, regarding R⁶ being a 5-membered monocyclic heteroaryl which contains at least one S ring atom, the expression “one R⁷ is attached to the C ring atom at position 2 relative to the ring atom by which R⁶ is bound to the remainder of the compound and one R⁷ is attached to the C ring atom at position 5 relative to the ring atom by which R⁶ is bound to the remainder of the compound” as used herein (and similar expressions) preferably means that to each of the two C ring atoms of R⁶ which are directly adjacent to the ring atom by which R⁶ is attached to the remainder of the compound one R⁷ is bound. For example, applying the above expression (“one R⁷ is attached to the C ring atom at position 2 relative to the ring atom by which R⁶ is bound to the remainder of the compound and one R⁷ is attached to the C ring atom at position 5 relative to the ring atom by which R⁶ is bound to the remainder of the compound”) to the case where R⁶ is 3-thienyl (thus, the yl position is the ring carbon at position 3 relative to the S ring atom) substituted with at least two R⁷ groups, it follows that these R⁷ groups are at position 2 and 4 of the 3-thienyl group, as shown in the following formula:

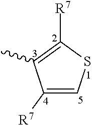

wherein [symbol] represents the bond by which R⁶ is bound to the remainder of the compound.

The expression “ [symbol] represents the bond by which R⁶ is bound to the remainder of the compound” as used herein refers to the bond through which R⁶ is attached to the remainder of the compound (i.e., attached to either (i) L in case L is not a bond or (ii) the nitrogen atom of the —C(=E)N(R^{4a})(R^{4b})C— of formula (I), (Ia), (II), (III), (IV), (V), (Va), (VI), (VII), (VIIa), (VIII), (IX), (X), (XI), and (XII) in case L is a bond). For example, in case R⁶ is

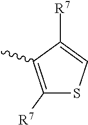

and L is (i) methylene or (ii) a bond, a compound of formula (II) has the following structure (A1) and (A2), respectively:

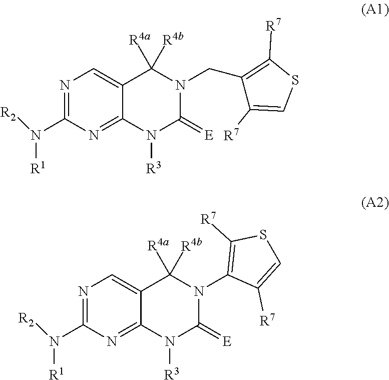

Similar terms such as “ [symbol] represents the bond by which R^{1a} is bound to the remainder of the compound” as used herein are to be interpreted in an analogous manner.

The term “non-symmetrical” as used herein (for example, in connection with R^{1a}) preferably means that the moiety concerned, in particular a non-symmetrical cycloalkyl or heterocyclyl group, relative to its point of attachment to the remainder of the compound, is non-symmetrical as such (e.g., 1,4-oxazepan-4-yl) and/or has a substitution pattern which is non-symmetrical (e.g., 3-oxopiperazin-1-yl or 3-methylpiperazin-1-yl). For example, relative to its point of attachment to the remainder of the compound, a symmetrical group has symmetry plane (as in 4-methylpiperazinyl), whereas a non-symmetrical group does not have a symmetry plane. A non-symmetrical group may have an asymmetric atom (e.g., a chiral C atom), such as in 2-methylmorpholin-4-yl, but does not necessarily have an asymmetric atom (such as in 3-oxopiperazin-1-yl). Exemplary groups which are non-symmetrical include the following:

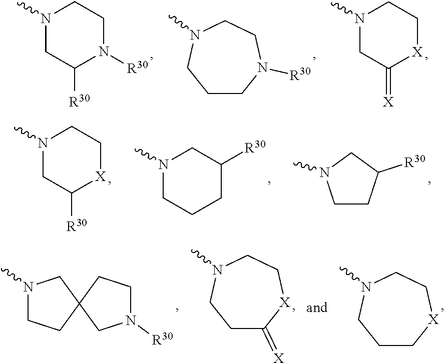

and/or also include the analogous C-linked moities (as applicable).
wherein R³⁰ and X are as defined herein; and [symbol] represents the bond by which the non-symmetrical group is bound to the remainder of the compound.

Particular groups which are non-symmetrical include the following:

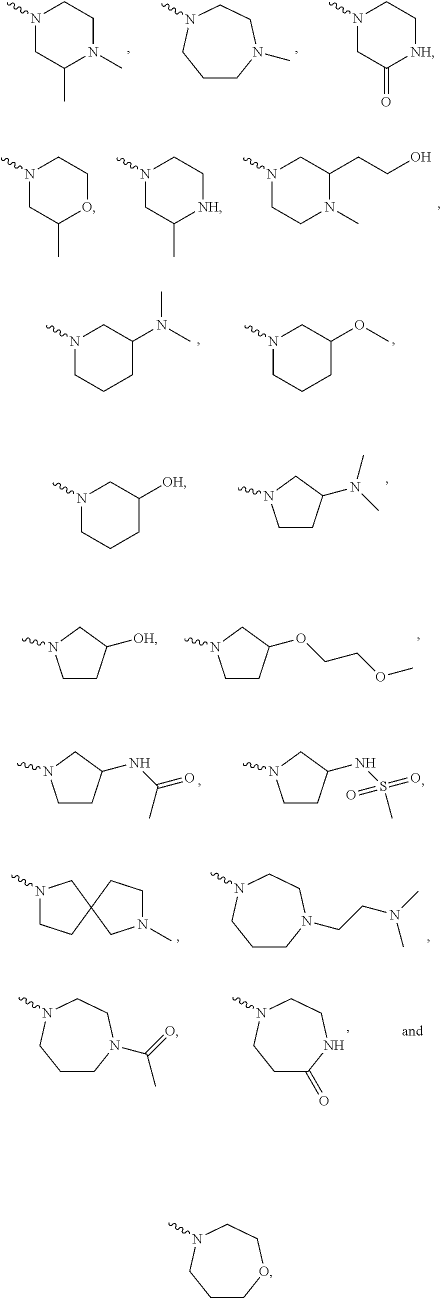

and/or also include the following:

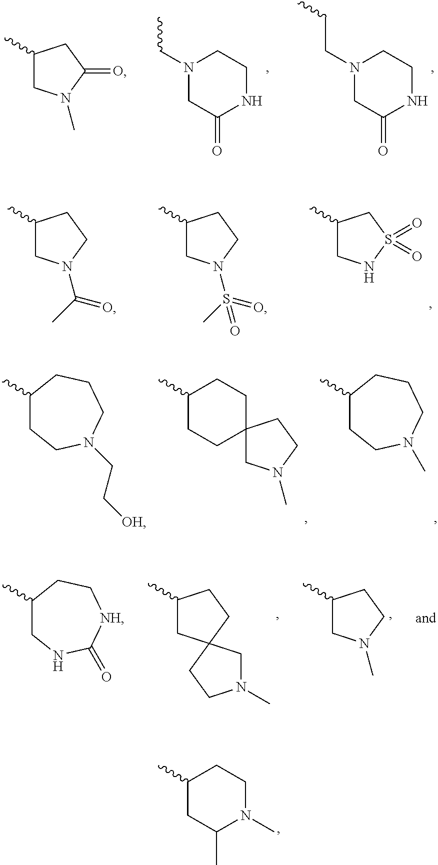

wherein R³⁰ and X are as defined herein; and [symbol] represents the bond by which the non-symmetrical group is bound to the remainder of the compound.

The expression “adjacent ring atoms” as used herein, like in “the C ring atom and the S ring atom are adjacent ring atoms” preferably means that these two ring atoms share a common bond and, thus, are directly bound to each other. For example, in the structure shown below (i.e., a 3-thienyl group which is substituted with R⁷ at position 4), the C ring atom at position 2 and the S ring atom are adjacent ring atoms, whereas the C ring atom at position 4 and the S ring atom are separated by a C ring atom:

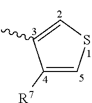

wherein [symbol] represents the bond by which R⁶ is bound to the remainder of the compound.

The expression “the S ring atom of R⁶ is not adjacent to the ring atom by which R⁶ is bound to the remainder of the compound” as used herein preferably means that the S ring atom of R⁶ is separated from the ring atom by which R⁶ is bound to the remainder of the compound (i.e., from the yl position of R⁶) by at least one ring atom. For example, in case R⁶ is thienyl optionally substituted with one R⁷, the expression “the S ring atom of R⁶ is not adjacent to the ring atom by which R⁶ is bound to the remainder of the compound” encompasses the following structures:

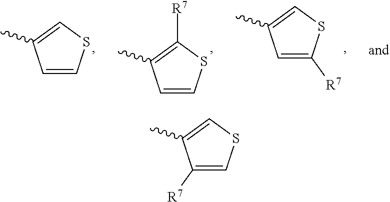

but excludes, inter alia, the following structures:

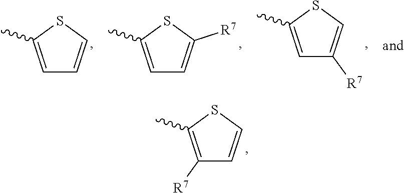

wherein [symbol] represents the bond by which R⁶ is bound to the remainder of the compound.

In accordance with the IUPAC nomenclature, preferably the numbering of a substituted heterocyclyl group starts at the ring heteroatom and continues in such a way that the numbers of the substituents are as low as possible. For example, the compound shown below has the following numbering of the ring atoms and the following name:

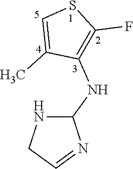

### N-(2-fluoro-4-methylthiophen-3-yl)-2,5-dihydro-1H-imidazol-2-amine

The term “optionally substituted” indicates that one or more (such as 1 to the maximum number of hydrogen atoms bound to a group, e.g., 1, 2, 3, 4, 5, 6, 7, 8, 9, or up to 10, such as between 1 to 5, 1 to 4, or 1 to 3, or 1 or 2) hydrogen atom(s) may be replaced with a group (i.e., a 1^{st} level substituent) different from hydrogen such as alkyl (preferably, C₁₋₆ alkyl), alkenyl (preferably, C₂₋₆ alkenyl), alkynyl (preferably, C₂₋₆ alkynyl), aryl (preferably, 6- to 14-membered aryl), heteroaryl (preferably, 3- to 14-membered heteroaryl), cycloalkyl (preferably, 3- to 14-membered cycloalkyl), heterocyclyl (preferably, 3- to 14-membered heterocyclyl), halogen, —CN, azido, —NO₂, —OR⁷¹, —N(R⁷²)(R⁷³), —S(O)₀₋₂R⁷¹, —S(O)₁₋₂OR⁷¹, —OS(O)₁₋₂R⁷¹, —OS(O)₁₋₂OR⁷¹, —S(O)₁₋₂N(R⁷²)(R⁷³), —OS(O)₁₋₂N(R⁷²)(R⁷³), —N(R⁷¹)S(O)₁₋₂R⁷¹, —NR⁷¹S(O)₁₋₂OR⁷¹, —NR⁷¹S(O)₁₋₂N(R⁷²)(R⁷³), —OP(O)(OR⁷¹)₂, —C(═X¹)R⁷¹, —C(═X¹)X¹R⁷¹, —X¹C(═X¹)R⁷¹, and —X¹C(═X¹)X¹R⁷¹, and/or any two 1^{st} level substituents which are bound to the same carbon atom of a cycloalkyl or heterocyclyl group may join together to form ═X¹, wherein each of the alkyl, alkenyl, alkynyl, aryl, heteroaryl, cycloalkyl, and heterocyclyl groups of the 1^{st} level substituent may themselves be substituted by one or more (e.g., one, two or three) substituents (i.e., a 2^{nd} level substituent) selected from the group consisting of C₁₋₆ alkyl, C₂₋₆ alkenyl, C₂₋₆ alkynyl, 6- to 14-membered aryl, 3- to 14-membered heteroaryl, 3- to 14-membered cycloalkyl, 3- to 14-membered heterocyclyl, halogen, —CF₃, —CN, azido, —NO₂, —OR⁸¹, —N(R⁸²)(R⁸³), —S(O)₀₋₂R⁸¹, —S(O)₁₋₂OR⁸¹, —OS(O)₁₋₂R⁸¹, —OS(O)₁₋₂OR⁸¹, —S(O)₁₋₂N(R⁸²)(R⁸³), —OS(O)₁₋₂N(R⁸²)(R⁸³), —N(R⁸¹)S(O)₁₋₂R⁸¹, —NR⁸¹S(O)₁₋₂OR⁸¹, —NR⁸¹S(O)₁₋₂N(R⁸²)(R⁸³), —OP(O)(OR⁸¹)₂, —C(═X²)R⁸¹, —C(═X²)X²R⁸¹, —X²C(═X²)R⁸¹, and —X²C(═X²)X²R⁸¹, and/or any two 2^{nd} level substituents which are bound to the same carbon atom of a cycloalkyl or heterocyclyl group being a 1^{st} level substituent may join together to form ═X², wherein each of the C₁₋₆ alkyl, C₂₋₆ alkenyl, C₂₋₆ alkynyl, 6- to 14-membered aryl, 3- to 14-membered heteroaryl, 3- to 14-membered cycloalkyl, 3- to 14-membered heterocyclyl groups of the 2^{nd} level substituent is optionally substituted with one or more (e.g., one, two or three) substituents (i.e., a 3^{rd} level substituent) independently selected from the group consisting of C₁₋₃ alkyl, halogen, —CF₃, —CN, azido, —NO₂, —OH, —O(C₁₋₃ alkyl), —OCF₃, —S(C₁₋₃ alkyl), —NH₂, —NH(C₁₋₃ alkyl), —N(C₁₋₃ alkyl)₂, —NHS(O)₂(C₁₋₃ alkyl), —S(O)₂NH_{2-z}(C₁₋₃ alkyl)_{z}, —C(═O)OH, —C(═O)O(C₁₋₃ alkyl), —C(═O)NH_{2-z}(C₁₋₃ alkyl)_{z}, —NHC(═O)(C₁₋₃ alkyl), —NHC(═NH)NH_{z-2}(C₁₋₃ alkyl)_{z}, and —N(C₁₋₃ alkyl)C(═NH)NH_{2-z}(C₁₋₃ alkyl)_{z}, wherein each z is independently 0, 1, or 2 and each C₁₋₃ alkyl is independently methyl, ethyl, propyl or isopropyl, and/or any two 3^{rd} level substituents which are bound to the same carbon atom of a 3- to 14-membered cycloalkyl or heterocyclyl group being a 2^{nd} level substituent may join together to form ═O, ═S, ═NH, or ═N(C₁₋₃ alkyl);

wherein
each of R⁷¹, R⁷², and R⁷³ is independently selected from the group consisting of H, C₁₋₆ alkyl, C₂₋₆ alkenyl, C₂₋₆ alkynyl, 3- to 7-membered cycloalkyl, 5- or 6-membered aryl, 5- or 6-membered heteroaryl, and 3- to 7-membered heterocyclyl, wherein each of the C₁₋₆ alkyl, C₂₋₆ alkenyl, C₂₋₆ alkynyl, 3- to 7-membered cycloalkyl, 5- or 6-membered aryl, 5- or 6-membered heteroaryl, and 3- to 7-membered heterocyclyl groups is optionally substituted with one, two or three substituents independently selected from the group consisting of C₁₋₃ alkyl, halogen, —CF₃, —CN, azido, —NO₂, —OH, —O(C₁₋₃ alkyl), —OCF₃, ═O, —S(C₁₋₃ alkyl), —NH₂, —NH(C₁₋₃ alkyl), —N(C₁₋₃ alkyl)₂, —NHS(O)₂(C₁₋₃ alkyl), —S(O)₂NH_{2-z}(C₁₋₃ alkyl)_{z}, —C(═O)(C₁₋₃ alkyl), —C(═O)OH, —C(═O)O(C₁₋₃ alkyl), —C(═O)NH_{2-z}(C₁₋₃ alkyl)_{z}, —NHC(═O)(C₁₋₃ alkyl), —NHC(═NH)NH_{z-2}(C₁₋₃ alkyl)_{z}, and —N(C₁₋₃ alkyl)C(═NH)NH_{2-z}(C₁₋₃ alkyl)_{z}, wherein each z is independently 0, 1, or 2 and each C₁₋₃ alkyl is independently methyl, ethyl, propyl or isopropyl;
each of R⁸¹, R⁸², and R⁸³ is independently selected from the group consisting of H, C₁₋₄ alkyl, C₂₋₄ alkenyl, C₂₋₄ alkynyl, 3- to 6-membered cycloalkyl, 5- or 6-membered aryl, 5- or 6-membered heteroaryl, and 3- to 6-membered heterocyclyl, wherein each of the C₁₋₄ alkyl, C₂₋₄ alkenyl, C₂₋₄ alkynyl, 3- to 6-membered cycloalkyl, 5- or 6-membered aryl, 5- or 6-membered heteroaryl, and 3- to 6-membered heterocyclyl groups is optionally substituted with one, two or three substituents independently selected from the group consisting of C₁₋₃ alkyl, halogen, —CF₃, —CN, azido, —NO₂, —OH, —O(C₁₋₃ alkyl), —OCF₃, ═O, —S(C₁₋₃ alkyl), —NH₂, —NH(C₁₋₃ alkyl), —N(C₁₋₃ alkyl)₂, —NHS(O)₂(C₁₋₃ alkyl), —S(O)₂NH_{2-z}(C₁₋₃ alkyl)_{z}, —C(═O)(C₁₋₃ alkyl), —C(═O)OH, —C(═O)O(C₁₋₃ alkyl), —C(═O)NH_{2-z}(C₁₋₃ alkyl)_{z}, —NHC(═O)(C₁₋₃ alkyl), —NHC(═NH)NH_{z-2}(C₁₋₃ alkyl)_{z}, and —N(C₁₋₃ alkyl)C(═NH)NH_{2-z}(C₁₋₃ alkyl)_{z}, wherein each z is independently 0, 1, or 2 and each C₁₋₃ alkyl is independently methyl, ethyl, propyl or isopropyl; and each of XI and X² is independently selected from O, S, and N(R⁸⁴), wherein R⁸⁴ is H or C₁₋₃ alkyl.

Typical 1^{st} level substituents are preferably selected from the group consisting of C₁₋₆ alkyl, C₂₋₆ alkenyl, C₂₋₆ alkynyl, 6- to 14-membered (such as 6- to 10-membered) aryl, 3- to 14-membered (such as 5- or 6-membered) heteroaryl, 3- to 14-membered (such as 3- to 7-membered) cycloalkyl, 3- to 14-membered (such as 3- to 7-membered) heterocyclyl, halogen, —CN, azido, —NO₂, —OR⁷¹, —N(R⁷²)(R⁷³), —S(O)₀₋₂R⁷¹, —S(O)₁₋₂OR⁷¹, —OS(O)₁₋₂R⁷¹, —OS(O)₁₋₂OR⁷¹, —S(O)₁₋₂N(R⁷²)(R⁷³), —OS(O)₁₋₂N(R⁷²)(R⁷³), —N(R⁷¹)S(O)₁₋₂R⁷¹, —NR⁷¹S(O)₁₋₂OR⁷¹, —C(═X¹)R⁷¹, —C(═X¹)X¹R⁷¹, —X¹C(═X¹)R⁷¹, and —X¹C(═X¹)X¹R⁷¹, such as C₁₋₄ alkyl, C₂₋₄ alkenyl, C₂₋₄ alkynyl, 6-membered aryl, 5- or 6-membered heteroaryl, 3- to 7-membered cycloalkyl, 3- to 7-membered (such as 5- or 6-membered) heterocyclyl, halogen, —CF₃, —CN, azido, —NO₂, —OH, —O(C₁₋₃ alkyl), —S(C₁₋₃ alkyl), —NH₂, —NH(C₁₋₃ alkyl), —N(C₁₋₃ alkyl)₂, —NHS(O)₂(C₁₋₃ alkyl), —S(O)₂NH_{2-z}(C₁₋₃ alkyl)_{z}, —C(═O)OH, —C(═O)O(C₁₋₃ alkyl), —C(═O)NH_{2-z}(C₁₋₃ alkyl)_{z}, —NHC(═O)(C₁₋₃ alkyl), —NHC(═NH)NH_{z-2}(C₁₋₃ alkyl)_{z}, and —N(C₁₋₃ alkyl)C(═NH)NH_{2-z}(C₁₋₃ alkyl)_{z}, wherein each z is independently 0, 1, or 2 and each C₁₋₃ alkyl is independently methyl, ethyl, propyl or isopropyl; wherein X¹ is independently selected from O, S, NH and N(CH₃); and each of R⁷¹, R⁷², and R⁷³ is as defined above or, preferably, is independently selected from the group consisting of H, C₁₋₄ alkyl, C₂₋₄ alkenyl, C₂₋₄ alkynyl, 5- or 6-membered cycloalkyl, 5- or 6-membered aryl, 5- or 6-membered heteroaryl, and 5- or 6-membered heterocyclyl, wherein each of the alkyl, alkenyl, alkynyl, cycloalkyl, aryl, heteroaryl, and heterocyclyl groups is optionally substituted with one, two or three substituents independently selected from the group consisting of C₁₋₃ alkyl, halogen, —CF₃, —CN, azido, —NO₂, —OH, —O(C₁₋₃ alkyl), —S(C₁₋₃ alkyl), —NH₂, —NH(C₁₋₃ alkyl), —N(C₁₋₃ alkyl)₂, —NHS(O)₂(C₁₋₃ alkyl), —S(O)₂NH_{2-z}(C₁₋₃ alkyl)_{z}, —C(═O)OH, —C(═O)O(C₁₋₃ alkyl), —C(═O)NH_{2-z}(C₁₋₃ alkyl)_{z}, —NHC(═O)(C₁₋₃ alkyl), —NHC(═NH)NH_{z-2}(C₁₋₃ alkyl)_{z}, and —N(C₁₋₃ alkyl)C(═NH)NH_{2-z}(C₁₋₃ alkyl)_{z}, wherein each z is independently 0, 1, or 2 and each C₁₋₃ alkyl is independently methyl, ethyl, propyl or isopropyl. Particular examples of 1^{st} level substituents are independently selected from the group consisting of C₁₋₃ alkyl, phenyl, imidazolyl, thiazolyl, cyclopentyl, cyclohexyl, dihydrothiazolyl, thiazolidinyl, halogen, —CF₃, —CN, —OH, —O(C₁₋₃ alkyl), —S(C₁₋₃ alkyl), —NH₂, —NH(C₁₋₃ alkyl), —N(C₁₋₃ alkyl)₂, —NHS(O)₂(C₁₋₃ alkyl), —C(═O)OH, —C(═O)O(C₁₋₃ alkyl), —C(═O)NH_{2-z}(C₁₋₃ alkyl)_{z}, —NHC(═O)(C₁₋₃ alkyl), —NHC(═NH)NH_{z-2}(C₁₋₃ alkyl)_{z}, and —N(C₁₋₃ alkyl)C(═NH)NH_{2-z}(C₁₋₃ alkyl)_{z}, wherein each z is independently 0, 1, or 2 and each C₁₋₃ alkyl is independently methyl, ethyl, propyl or isopropyl. Particularly preferred 1^{st} level substituents are independently selected from the group consisting of C₁₋₃ alkyl, phenyl, thiazolidinyl, halogen (such as F, Cl, or Br), —NH₂, —NHS(O)₂(C₁₋₃ alkyl), —NHC(═O)(C₁₋₃ alkyl), and —NHC(═NH)NH_{z-2}(C₁₋₃ alkyl)_{z}, wherein z is 0, 1, or 2 and each C₁₋₃ alkyl is independently methyl, ethyl, propyl or isopropyl.

Typical 2^{nd} level substituents are preferably selected from the group consisting of C₁₋₄ alkyl, C₂₋₄ alkenyl, C₂₋₄ alkynyl, 6- or 10-membered aryl, 5- or 6-membered heteroaryl, 5- or 6-membered cycloalkyl, 5- or 6-membered heterocyclyl, halogen, ═O, ═S, —CF₃, —CN, azido, —NO₂, —OH, —O(C₁₋₃ alkyl), —S(C₁₋₃ alkyl), —NH₂, —NH(C₁₋₃ alkyl), —N(C₁₋₃ alkyl)₂, —NHS(O)₂(C₁₋₃ alkyl), —S(O)₂NH_{2-z}(C₁₋₃ alkyl)_{z}, —C(═O)OH, —C(═O)O(C₁₋₃ alkyl), —C(═O)NH_{2-z}(C₁₋₃ alkyl)_{z}, —NHC(═O)(C₁₋₃ alkyl), —NHC(═NH)NH_{z-2}(C₁₋₃ alkyl)_{z}, and —N(C₁₋₃ alkyl)C(═NH)NH_{2-z}(C₁₋₃ alkyl)_{z}, wherein each z is independently 0, 1, or 2 and each C₁₋₃ alkyl is independently methyl, ethyl, propyl or isopropyl. Particular examples of 2^{nd} level substituents are independently selected from the group consisting of C₁₋₃ alkyl, phenyl, 5- or 6-membered heteroaryl, 5- or 6-membered cycloalkyl, 5- or 6-membered heterocyclyl, halogen, ═O, ═S, —CF₃, —CN, —OH, —O(C₁₋₃ alkyl), —S(C₁₋₃ alkyl), —NH₂, —NH(C₁₋₃ alkyl), —N(C₁₋₃ alkyl)₂, —NHS(O)₂(C₁₋₃ alkyl), —C(═O)OH, —C(═O)O(C₁₋₃ alkyl), —C(═O)NH_{2-z}(C₁₋₃ alkyl)_{z}, —NHC(═O)(C₁₋₃ alkyl), —NHC(═NH)NH_{z-2}(C₁₋₃ alkyl)_{z}, and —N(C₁₋₃ alkyl)C(═NH)NH_{2-z}(C₁₋₃ alkyl)_{z}, wherein each z is independently 0, 1, or 2 and each C₁₋₃ alkyl is independently methyl, ethyl, propyl or isopropyl. Particularly preferred 2^{nd} level substituents are independently selected from the group consisting of methyl, ethyl, propyl, isopropyl, phenyl, ═O, and ═S.

Typical 3^{rd} level substituents are preferably selected from the group consisting of C₁₋₃ alkyl, phenyl, halogen, —CF₃, —OH, —OCH₃, —SCH₃, —NH_{2-z}(CH₃)_{z}, —C(═O)OH, and —C(═O)OCH₃, wherein z is 0, 1, or 2 and C₁₋₃ alkyl is methyl, ethyl, propyl or isopropyl. Particularly preferred 3^{rd} level substituents are selected from the group consisting of methyl, ethyl, propyl, isopropyl, halogen (such as F, Cl, or Br), and —CF₃, such as halogen (e.g., F, Cl, or Br), and —CF₃.

The term “optional” or “optionally” as used herein means that the subsequently described event, circumstance or condition may or may not occur, and that the description includes instances where said event, circumstance, or condition occurs and instances in which it does not occur.

“Isomers” are compounds having the same molecular formula but differ in structure (“structural isomers”) or in the geometrical (spatial) positioning of the functional groups and/or atoms (“stereoisomers”). “Enantiomers” are a pair of stereoisomers which are non-superimposable mirror-images of each other. A “racemic mixture” or “racemate” contains a pair of enantiomers in equal amounts and is denoted by the prefix (+). “Diastereomers” are stereoisomers which are non-superimposable and which are not mirror-images of each other. “Tautomers” are structural isomers of the same chemical substance that spontaneously and reversibly interconvert into each other, even when pure, due to the migration of individual atoms or groups of atoms; i.e., the tautomers are in a dynamic chemical equilibrium with each other. An example of tautomers are the isomers of the keto-enol-tautomerism. “Conformers” are stereoisomers that can be interconverted just by rotations about formally single bonds, and include—in particular—those leading to different 3-dimensional forms of (hetero)cyclic rings, such as chair, half-chair, boat, and twist-boat forms of cyclohexane.

In case a structural formula shown in the present application can be interpreted to encompass more than one isomer, said structural formula, unless explicitly stated otherwise, encompasses all possible isomers and, hence, each individual isomer. For example, a compound of formula (I), wherein R^{4a} is H and R^{4b} is methyl encompasses both isomers, e.g., the isomer having the following formula (B1) and the isomer having the following formula (B2):

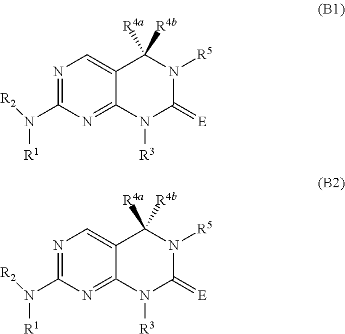

Furthermore, a compound of formula (I), wherein R⁶ is 1-azabicyclo[2.2.2]oct-3-yl (optionally substituted with one or more R⁷ groups) encompasses both isomers, e.g., the isomer having the following formula (B3) and the isomer having the following formula (B4) (wherein n1 is 0, 1, 2, 3, or more):

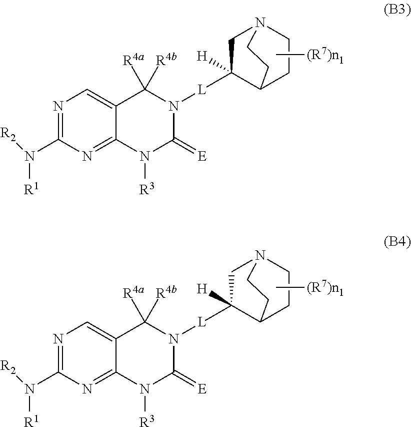

“Polymorphism” as referred to herein means that a solid material (such as a compound) is able to exist in more than one form or crystalline structure, i.e., “polymorphic modifications” or “polymorphic forms”. The terms “polymorphic modifications”, “polymorphic forms”, and “polymorphs” are used interchangeable in the present invention. According to the present invention, these “polymorphic modifications” include crystalline forms, amorphous forms, and solvates. Mainly, the reason for the existence of different polymorphic forms lies in the use of different conditions during the crystallization process, such as the following:
solvent effects (the packing of crystal may be different in polar and nonpolar solvents);
certain impurities inhibiting growth pattern and favour the growth of a metastable polymorphs;
the level of supersaturation from which material is crystallized (in which generally the higher the concentration above the solubility, the more likelihood of metastable formation);
temperature at which crystallization is carried out;
geometry of covalent bonds (differences leading to conformational polymorphism);
change in stirring conditions.

Polymorphic forms may have different chemical, physical, and/or pharmacological properties, including but not limited to, melting point, X-ray crystal and diffraction pattern, chemical reactivity, solubility, dissolution rate, vapor pressure, density, hygroscopicity, flowability, stability, compactability, and bioavailability. Polymorphic forms may spontaneously convert from a metastable form (unstable form) to the stable form at a particular temperature. According to Ostwald's rule, in general it is not the most stable but the least stable polymorph that crystallizes first. Thus, quality, efficacy, safety, processability and/or manufacture of a chemical compound, such as a compound of the present invention, can be affected by polymorphism. Often, the most stable polymorph of a compound (such as a compound of the present invention) is chosen due to the minimal potential for conversion to another polymorph. However, a polymorphic form which is not the most stable polymorphic form may be chosen due to reasons other than stability, e.g. solubility, dissolution rate, and/or bioavailability.

The term “crystalline form” of a material as used herein means that the smallest components (i.e., atoms, molecule or ions) of said material form crystal structures. A “crystal structure” as referred to herein means a unique three-dimensional arrangement of atoms or molecules in a crystalline liquid or solid and is characterized by a pattern, a set of atoms arranged in a particular manner, and a lattice exhibiting long-range order and symmetry. A lattice is an array of points repeating periodically in three dimensions and patterns are located upon the points of a lattice. The subunit of the lattice is the unit cell. The lattice parameters are the lengths of the edges of a unit cell and the angles between them. The symmetry properties of the crystal are embodied in its space group. In order to describe a crystal structure the following parameters are required: chemical formula, lattice parameters, space group, the coordinates of the atoms and occupation number of the point positions.

The term “amorphous form” of a material as used herein means that the smallest components (i.e., atoms, molecule or ions) of said material are not arranged in a lattice but are arranged randomly. Thus, unlike crystals in which a short-range order (constant distances to the next neighbour atoms) and a long-range order (periodical repetition of a basic lattice) exist, only a short-range order exists in an amorphous form.

The term “complex of a compound” as used herein refers to a compound of higher order which is generated by association of the compound with one or more other molecules. Exemplary complexes of a compound include, but are not limited to, solvates, clusters, and chelates of said compound.

The term “solvate” as used herein refers to an addition complex of a dissolved material in a solvent (such as an organic solvent (e.g., an aliphatic alcohol (such as methanol, ethanol, n-propanol, isopropanol), acetone, acetonitrile, ether, and the like), water or a mixture of two or more of these liquids), wherein the addition complex exists in the form of a crystal or mixed crystal. The amount of solvent contained in the addition complex may be stoichiometric or non-stoichiometric. A “hydrate” is a solvate wherein the solvent is water.

In isotopically labeled compounds one or more atoms are replaced by a corresponding atom having the same number of protons but differing in the number of neutrons. For example, a hydrogen atom may be replaced by a deuterium atom. Exemplary isotopes which can be used in the compounds of the present invention include deuterium, ¹¹C, ¹³C, ¹⁴C, ¹⁵N, ¹⁸F, ³²P, ³²S, ³⁵S, ³⁶Cl, and ¹²⁵I.

The expression “amino protecting group” as used herein preferably refers to any group by which an amino group contained in a compound can be transferred into a less reactive (i.e., protected) amino group. Preferably, amino protecting groups can be incorporated into the corresponding compound under mild conditions, in a chemoselective and/or regioselective manner, and/or in good yields. Furthermore, the amino protecting groups should be stable under the conditions to which the protected compound is to be subjected (e.g., the conditions of the desired reaction and/or purification conditions). Preferably, the amino protecting groups should minimize the risk of racemization of a stereogenic center, when present in the compound. In one embodiment, the amino protecting groups should be removable from the protected compound under mild conditions and in a selective manner such that the deprotected compound is obtained in high yields. Exemplary amino protecting groups include tert-butyloxycarbonyl (BOC), 9-fluorenylmethoxycarbonyl (FMOC), benzyloxycarbonyl (Cbz), p-methoxybenzylcarbonyl (MOZ), acetyl (Ac), trifluoroacetyl, benzoyl (Bz), benzyl (Bn), p-methoxybenzyl (PMB), 3,4-dimethoxyphenyl (DMPM), p-methoxyphenyl (PMP), 2,2,2-trichloroethoxycarbonyl (Troc), triphenylmethyl (trityl; Tr), toluenesulfonyl (tosyl; Ts), para-bromophenylsulfonyl (brosyl), 4-nitrobenzenesulfonyl (nosyl), and 2-nitrophenylsulfenyl (Nps).

The term “half-life” relates to the period of time which is needed to eliminate half of the activity, amount, or number of molecules. In the context of the present invention, the half-life of a compound disclosed herein (eg a compound of formula (I), (Ia), (III), (IV), (V), (Va), (VI), (VII), (VIIa), (VIII), (IX), (X), (XI), or (XII)) is indicative for the stability of said compound.

The terms “subject”, “patient”, “individual”, or “animal” relate to multicellular animals, such as vertebrates. For example, vertebrates in the context of the present invention are mammals, birds (e.g., poultry), reptiles, amphibians, bony fishes, and cartilaginous fishes, in particular domesticated animals of any of the foregoing as well as animals (in particular vertebrates) in captivity such as animals (in particular vertebrates) of zoos. Mammals in the context of the present invention include, but are not limited to, humans, non-human primates, domesticated mammals, such as dogs, cats, sheep, cattle, goats, pigs, horses etc., laboratory mammals such as mice, rats, rabbits, guinea pigs, etc. as well as mammals in captivity such as mammals of zoos. The term “animal” as used herein also includes humans. Particular non-limiting examples of birds include domesticated poultry, and include birds such as chickens, turkeys, ducks, geese, guinea fowl, pigeons, pheasants etc.; while particular non-limiting examples of bony or cartilaginous fish include those suitable for cultivation by aquiculture, and include bony fish such as salmon, trout, perch, carp, cat-fish, etc.

The compound YKL-05-099 (3-(2-chloro-6-methylphenyl)-7-((2-methoxy-4-(1-methylpiperidin-4-yl)phenyl)amino)-1-(5-methoxypyridin-2-yl)-3,4-dihydropyrimido[4,5-d]pyrimidin-2(1H)-one), herein referred to as compound PY1, has the following structure:

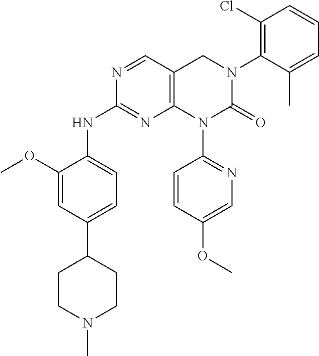 [SMILES: COc1ccc(N2C(=O)N(c3c(C)cccc3Cl)Cc3cnc(Nc4ccc(C5CCN(C)CC5)cc4OC)nc32)nc1]

### Compounds

In a first aspect, and as may be further described, defined, claimed or otherwise disclosed herein, the present invention provides a compound selected from the group consisting of a kinase inhibitor of the formula (I):

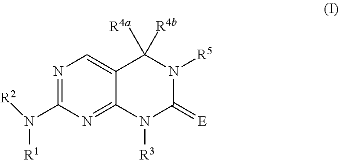

and solvates, salts, N-oxides, complexes, polymorphs, crystalline forms, racemic mixtures, diastereomers, enantiomers, tautomers, conformers, isotopically labeled forms, prodrugs, and combinations thereof;
wherein:
R¹ is -Q-R^{1a};
R^{1a} is selected from the group consisting of H, alkyl, alkenyl, alkynyl, cycloalkyl, aryl, heterocyclyl, heteroaryl, halogen, —CN, azido, —NO₂, —OR¹¹, —N(R¹²)(R¹³), —N(R¹¹)(OR¹¹), —S(O)₀₋₂R¹¹, —S(O)₁₋₂OR¹¹, —OS(O)₁₋₂R¹¹, —OS(O)₁₋₂OR¹¹, —S(O)₁₋₂N(R¹²)(R¹³), —OS(O)₁₋₂N(R¹²)(R¹³), —N(R¹¹)S(O)₁₋₂R¹¹, —NR¹¹S(O)₁₋₂OR¹¹, —NR¹¹S(O)₁₋₂N(R¹²)(R¹³), —P(O)(OR¹¹)₂, —OP(O)(OR¹¹)₂, —C(═X)R¹¹, —C(═X)XR¹¹, —XC(═X)R¹¹, and —XC(═X)XR¹¹, wherein each of the alkyl, alkenyl, alkynyl, cycloalkyl, aryl, heterocyclyl, and heteroaryl groups is optionally substituted with one or more independently selected R³⁰;
Q is selected from the group consisting of a bond, alkyl, alkenyl, alkynyl, cycloalkyl, aryl, heterocyclyl, heteroaryl, wherein each of the alkyl, alkenyl, alkynyl, cycloalkyl, aryl, heterocyclyl, and heteroaryl groups is optionally substituted with one or more independently selected R³⁰;

### R² is H;

R³ is selected from the group consisting of a bond, H, alkyl, alkenyl, alkynyl, cycloalkyl, aryl, heterocyclyl, heteroaryl, halogen, —CN, azido, —NO₂, —OR¹¹, —N(R¹²)(R¹³), —N(R¹¹)(OR¹¹), —S(O)₀₋₂R¹¹, —S(O)₁₋₂OR¹¹, —OS(O)₁₋₂R¹¹, —OS(O)_{1- 2}OR¹¹, —S(O)₁₋₂N(R¹²)(R¹³), —OS(O)₁₋₂N(R¹²)(R¹³), —N(R¹¹)S(O)₁₋₂R¹¹, —NR¹¹S(O)₁₋₂OR¹¹, —NR¹¹S(O)₁₋₂N(R¹²)(R¹³), —P(O)(OR¹¹)₂, —OP(O)(OR¹¹)₂, —C(═X)R¹¹, —C(═X)XR¹¹, —XC(═X)R¹¹, and —XC(═X)XR¹¹, wherein each of the alkyl, alkenyl, alkynyl, cycloalkyl, aryl, heterocyclyl, and heteroaryl groups is optionally substituted with one or more independently selected R³⁰; optionally R¹ and R³ may join together via a group L′ to form a moiety R¹-L′-R³, preferably to form a moiety Q-L′-R³, wherein L′ is selected from the group consisting of a bond, alkylene, alkenylene, alkynylene, —(CH₂)ₚ-[Y—(CH₂)_{q}]ᵣ, alkenylene —[Y—(CH₂)_{q}]ᵣ—, wherein p is an integer between 1 and 10, q is an integer between 0 and 6, r is an integer between 1 and 3, wherein if q is 0 then r is 1; Y is independently selected from 0, S, and —N(R¹³)—; and each of the alkylene, alkenylene, alkynylene, —(CH₂)ₚ—, and —(CH₂)_{q}— groups is optionally substituted with one or more independently selected R³⁰;
each of R^{4a} and R^{4b} is independently selected from the group consisting of H, C₁₋₈ alkyl, and —(CH₂)ₛ—[O—(CH₂)ₜ]ᵤ—H wherein s is an integer between 0 and 7, t is an integer between 0 and 7, u is an integer between 1 and 3, wherein if t is 0 then u is 1, and the total number of carbon and oxygen atoms of the —(CH₂)ₛ—[O—(CH₂)ₜ]ᵤ—H does not exceed 8; or optionally R^{4a} and R^{4b} may join together to form, together with the carbon to which they are attached, C₃₋₆ cycloalkyl or a 4- to 6-membered heterocyclyl comprising at least one 0 as the only heteroatom element, wherein in case that the 4- to 6-membered heterocyclyl comprises more than one 0, different 0 are not directly bound to each other;
R⁵ is -L-R⁶;
L is selected from the group consisting of a bond, C₁₋₆ alkylene, C₂₋₆ alkenylene, C₂₋₆ alkynylene, and —(CH₂)ₘ—[Y—(CH₂)ₙ]ₒ—, wherein m is an integer between 1 and 6, n is an integer between 0 and 3, o is an integer between 1 and 3, wherein if n is 0 then o is 1; Y is independently selected from 0, S, and —N(R¹³)—; and each of the C₁₋₆ alkylene, C₂₋₆ alkenylene, C₂₋₆ alkynylene, —(CH₂)ₘ-, and —(CH₂)ₙ— groups is optionally substituted with one or two independently selected R³⁰;
R⁶ is heteroaryl or heterocyclyl each of which is optionally substituted with one or more independently selected R⁷;
R⁷ is independently selected from the group consisting of alkyl, alkenyl, alkynyl, cycloalkyl, aryl, heterocyclyl, heteroaryl, halogen, —CN, azido, —NO₂, —OR¹¹, —N(R¹²)(R¹³), —N(R¹¹)(OR¹¹), —S(O)₀₋₂R¹¹, —S(O)₁₋₂OR¹¹, —OS(O)₁₋₂R¹¹, —OS(O)₁₋₂OR¹¹, —S(O)₁₋₂N(R¹²)(R¹³), —OS(O)₁₋₂N(R¹²)(R¹³), —N(R¹¹)S(O)₁₋₂R¹¹, —NR¹¹S(O)₁₋₂OR¹¹, —NR¹¹S(O)₁₋₂N(R¹²)(R¹³), —P(O)(OR¹¹)₂, —OP(O)(OR¹¹)₂, —C(═X)R¹¹, —C(═X)XR¹¹, —XC(═X)R¹¹, and —XC(═X)XR¹¹, and/or any two R⁷ which are bound to the same atom of R⁶ being a heterocyclyl group may join together to form ═O, wherein each of the alkyl, alkenyl, alkynyl, cycloalkyl, aryl, heterocyclyl, and heteroaryl groups is optionally substituted with one or more independently selected R³⁰;

### E is O or S;

R¹¹ is independently selected from the group consisting of H, alkyl, alkenyl, alkynyl, cycloalkyl, aryl, heteroaryl, and heterocyclyl, wherein each of the alkyl, alkenyl, alkynyl, cycloalkyl, aryl, heteroaryl, and heterocyclyl groups is optionally substituted with one or more independently selected R³⁰;
each of R¹² and R¹³ is independently selected from the group consisting of H, alkyl, alkenyl, alkynyl, cycloalkyl, aryl, heteroaryl, and heterocyclyl, or R¹² and R¹³ may join together with the nitrogen atom to which they are attached to form the group —N═CR¹⁵R¹⁶, wherein each of the alkyl, alkenyl, alkynyl, cycloalkyl, aryl, heteroaryl, and heterocyclyl groups is optionally substituted with one or more independently selected R³⁰;
each of R¹⁵ and R¹⁶ is independently selected from the group consisting of H, alkyl, alkenyl, alkynyl, cycloalkyl, aryl, heteroaryl, heterocyclyl, and —NH_{y}R²⁰_{2-y}, or R¹⁵ and R¹⁶ may join together with the atom to which they are attached to form a ring which is optionally substituted with one or more independently selected R³⁰, wherein each of the alkyl, alkenyl, alkynyl, cycloalkyl, aryl, heteroaryl, and heterocyclyl groups is optionally substituted with one or more independently selected R³⁰;
y is an integer from 0 to 2;
R²⁰ is independently selected from the group consisting of alkyl, alkenyl, alkynyl, cycloalkyl, aryl, heteroaryl, and heterocyclyl, wherein each of the alkyl, alkenyl, alkynyl, cycloalkyl, aryl, heteroaryl, and heterocyclyl groups is optionally substituted with one or more independently selected R³⁰; and
R³⁰ is a 1^{st} level substituent and is, in each case, independently selected from the group consisting of alkyl, alkenyl, alkynyl, aryl, heteroaryl, cycloalkyl, heterocyclyl, halogen, —CN, azido, —NO₂, —OR⁷¹, —N(R⁷²)(R⁷³), —S(O)₀₋₂R⁷¹, —S(O)₁₋₂OR⁷¹, —OS(O)₁₋₂R⁷¹, —OS(O)₁₋₂OR⁷¹, —S(O)₁₋₂N(R⁷²)(R⁷³), —OS(O)₁₋₂N(R⁷²)(R⁷³), —N(R⁷¹)S(O)₁₋₂R⁷¹, —NR⁷¹S(O)₁₋₂OR⁷¹, —NR⁷¹S(O)₁₋₂N(R⁷²)(R⁷³), —OP(O)(OR⁷¹)₂, —C(═X¹)R⁷¹, —C(═X¹)X¹R⁷¹, —X¹C(═X¹)R⁷¹, and —X¹C(═X¹)X¹R⁷¹, and/or any two
R³⁰ which are bound to the same carbon atom of a cycloalkyl or heterocyclyl group may join together to form ═X¹, wherein each of the alkyl, alkenyl, alkynyl, aryl, heteroaryl, cycloalkyl, and heterocyclyl groups being a 1^{st} level substituent is optionally substituted by one or more 2^{nd} level substituents, wherein said 2^{nd} level substituent is, in each case, independently selected from the group consisting of C₁₋₆ alkyl, C₂₋₆ alkenyl, C₂₋₆ alkynyl, 3- to 14-membered aryl, 3- to 14-membered heteroaryl, 3- to 14-membered cycloalkyl, 3- to 14-membered heterocyclyl, halogen, —CF₃, —CN, azido, —NO₂, —OR⁸¹, —N(R⁸²)(R⁸³), —S(O)₀₋₂R⁸¹, —S(O)₁₋₂OR⁸¹, —OS(O)₁₋₂R⁸¹, —OS(O)₁₋₂OR⁸¹, —S(O)₁₋₂N(R⁸²)(R⁸³), —OS(O)₁₋₂N(R⁸²)(R⁸³), —N(R⁸¹)S(O)₁₋₂R⁸¹, —NR⁸¹S(O)₁₋₂OR⁸¹, —NR⁸¹S(O)₁₋₂N(R⁸²)(R⁸³), —OP(O)(OR⁸¹)₂, —C(═X²)R⁸¹, —C(═X²)X²R⁸¹, —X²C(═X²)R⁸¹, and —X²C(═X²)X²R⁸¹, and/or any two 2^{nd} level substituents which are bound to the same carbon atom of a cycloalkyl or heterocyclyl group being a 1^{st} level substituent may join together to form ═X², wherein each of the C₁₋₆ alkyl, C₂₋₆ alkenyl, C₂₋₆ alkynyl, 3- to 14-membered aryl, 3- to 14-membered heteroaryl, 3- to 14-membered cycloalkyl, 3- to 14-membered heterocyclyl groups being a 2^{nd} level substituent is optionally substituted with one or more 3rd level substituents, wherein said 3^{rd} level substituent is, in each case, independently selected from the group consisting of C₁₋₃ alkyl, halogen, —CF₃, —CN, azido, —NO₂, —OH, —O(C₁₋₃ alkyl), —OCF₃, —S(C₁₋₃ alkyl), —NH₂, —NH(C₁₋₃ alkyl), —N(C₁₋₃ alkyl)₂, —NHS(O)₂(C₁₋₃ alkyl), —S(O)₂NH_{2-z}(C₁₋₃ alkyl)_{z}, —C(═O)OH, —C(═O)O(C₁₋₃ alkyl), —C(═O)NH_{2-z}(C₁₋₃ alkyl)_{z}, —NHC(═O)(C₁₋₃ alkyl), —NHC(═NH)NH_{z-2}(C₁₋₃ alkyl)_{z}, and —N(C₁₋₃ alkyl)C(═NH)NH_{2-z}(C₁₋₃ alkyl)_{z}, wherein each z is independently 0, 1, or 2 and each C₁₋₃ alkyl is independently methyl, ethyl, propyl or isopropyl, and/or any two 3^{rd} level substituents which are bound to the same carbon atom of a 3- to 14-membered cycloalkyl or heterocyclyl group being a 2^{nd} level substituent may join together to form ═O, ═S, ═NH, or ═N(C₁₋₃ alkyl);
wherein
each of R⁷¹, R⁷², and R⁷³ is independently selected from the group consisting of H, C₁₋₆ alkyl, C₂₋₆ alkenyl, C₂₋₆ alkynyl, 3- to 7-membered cycloalkyl, 5- or 6-membered aryl, 5- or 6-membered heteroaryl, and 3- to 7-membered heterocyclyl, wherein each of the C₁₋₆ alkyl, C₂₋₆ alkenyl, C₂₋₆ alkynyl, 3- to 7-membered cycloalkyl, 5- or 6-membered aryl, 5- or 6-membered heteroaryl, and 3- to 7-membered heterocyclyl groups is optionally substituted with one, two or three substituents independently selected from the group consisting of C₁₋₃ alkyl, halogen, —CF₃, —CN, azido, —NO₂, —OH, —O(C₁₋₃ alkyl), —OCF₃, ═O, —S(C₁₋₃ alkyl), —NH₂, —NH(C₁₋₃ alkyl), —N(C₁₋₃ alkyl)₂, —NHS(O)₂(C₁₋₃ alkyl), —S(O)₂NH_{2-z}(C₁₋₃ alkyl)_{z}, —C(═O)(C₁₋₃ alkyl), —C(═O)OH, —C(═O)O(C₁₋₃ alkyl), —C(═O)NH_{2-z}(C₁₋₃ alkyl)_{z}, —NHC(═O)(C₁₋₃ alkyl), —NHC(═NH)NH_{z-2}(C₁₋₃ alkyl)_{z}, and —N(C₁₋₃ alkyl)C(═NH)NH_{2-z}(C₁₋₃ alkyl)_{z}, wherein each z is independently 0, 1, or 2 and each C₁₋₃ alkyl is independently methyl, ethyl, propyl or isopropyl;
each of R⁸¹, R⁸², and R⁸3 is independently selected from the group consisting of H, C₁₋₄ alkyl, C₂₋₄ alkenyl, C₂₋₄ alkynyl,
3- to 6-membered cycloalkyl, 5- or 6-membered aryl, 5- or 6-membered heteroaryl, and 3- to 6-membered heterocyclyl, wherein each of the C₁₋₄ alkyl, C₂₋₄ alkenyl, C₂₋₄ alkynyl, 3- to 6-membered cycloalkyl, 5- or 6-membered aryl, 5- or 6-membered heteroaryl, and 3- to 6-membered heterocyclyl groups is optionally substituted with one, two or three substituents independently selected from the group consisting of C₁₋₃ alkyl, halogen, —CF₃, —CN, azido, —NO₂, —OH, —O(C₁₋₃ alkyl), —OCF₃, ═O, —S(C₁₋₃ alkyl), —NH₂, —NH(C₁₋₃ alkyl), —N(C₁₋₃ alkyl)₂, —NHS(O)₂(C₁₋₃ alkyl), —S(O)₂NH_{2-z}(C₁₋₃ alkyl)_{z}, —C(═O)(C₁₋₃ alkyl), —C(═O)OH, —C(═O)O(C₁₋₃ alkyl), —C(═O)NH_{2-z}(C₁₋₃ alkyl)_{z}, —NHC(═O)(C₁₋₃ alkyl), —NHC(═NH)NH_{z-2}(C₁₋₃ alkyl)_{z}, and —N(C₁₋₃ alkyl)C(═NH)NH_{2-z}(C₁₋₃ alkyl)_{z}, wherein each z is independently 0, 1, or 2 and each C₁₋₃ alkyl is independently methyl, ethyl, propyl or isopropyl; and
each of X¹ and X² is independently selected from O, S, and N(R⁸⁴), wherein R⁸⁴ is H or C₁₋₃ alkyl;
optionally with the proviso that:
when E is O and either of (1) or (2) is true:
(1) R¹ and R³ do not join together via a group L′ to form a moiety R¹-L′-R³; or
(2) both of R^{4a} and R^{4b} are H, then either:
   (a) R⁶ is: (i) a 5-membered monocyclic heteroaryl which contains at least one S ring atom and which is substituted with one or more independently selected R⁷; (ii) a 5-membered monocyclic heteroaryl which contains at least two nitrogen atoms and which is substituted with one or more independently selected R⁷; or (iii) a 5-membered monocyclic heteroaryl which contains at least one nitrogen atom and at least one oxygen atom and which is substituted with one or more independently selected R⁷; or
   (b) R⁷ is independently selected from the group consisting of alkyl, alkenyl, alkynyl, cycloalkyl, aryl, heterocyclyl, heteroaryl, halogen, —CN, azido, —NO₂, —OR¹¹, —N(R¹²)(R¹³), —N(R¹¹)(OR¹¹), —S(O)₀₋₂R¹¹, —S(O)₁₋₂OR¹¹, —OS(O)₁₋₂R¹¹, —OS(O)₁₋₂OR¹¹, —S(O)₁₋₂N(R¹²)(R¹³), —OS(O)₁₋₂N(R¹²)(R¹³), —N(R¹¹)S(O)₁₋₂R¹¹, —NR¹¹S(O)₁₋₂OR¹¹, —NR¹¹S(O)₁₋₂N(R¹²)(R¹³), —P(O)(OR¹¹)₂, —OP(O)(OR¹¹)₂, —C(═X)R¹¹, —C(═X)XR¹¹, —XC(═X)R¹¹, and —XC(═X)XR¹¹, wherein each of the alkyl, alkenyl, alkynyl, cycloalkyl, aryl, heterocyclyl, and heteroaryl groups is optionally substituted with one or more independently selected R³⁰, wherein at least one of R⁷ is F and/or at least one of R⁷ is substituted with one or more F atoms.

In one embodiment of formula (I), when E is O and both of R^{4a} and R^{4b} are H, then R⁶ is: (i) a 5-membered monocyclic heteroaryl which contains at least one S ring atom and which is substituted with one or more independently selected R⁷; (ii) a 5-membered monocyclic heteroaryl which contains at least two nitrogen atoms and which is substituted with one or more independently selected R⁷; or (iii) a 5-membered monocyclic heteroaryl which contains at least one nitrogen atom and at least one oxygen atom and which is substituted with one or more independently selected R⁷. In a first particular of such embodiments, R⁶ is then a 5-membered monocyclic heteroaryl which contains at least one S ring atom and which is substituted with one or more independently selected R⁷. In a second particular of such embodiments, R⁶ is then a 5-membered monocyclic heteroaryl which contains at least two nitrogen atoms and which is substituted with one or more independently selected R⁷. In a third particular of such embodiments, R⁶ is then a 5-membered monocyclic heteroaryl which contains at least one nitrogen atom and at least one oxygen atom and which is substituted with one or more independently selected R⁷.

In a first additional (or alternative) embodiment of formula (I), when E is O and both of R^{4a} and R^{4b} are H, then R⁷ is independently selected from the group consisting of alkyl, alkenyl, alkynyl, cycloalkyl, aryl, heterocyclyl, heteroaryl, halogen, —CN, azido, —NO₂, —OR¹¹, —N(R¹²)(R¹³), —N(R¹¹)(OR¹¹), —S(O)₀₋₂R¹¹, —S(O)₁₋₂OR¹¹, —OS(O)₁₋₂R¹¹, —OS(O)₁₋₂OR¹¹, —S(O)₁₋₂N(R¹²)(R¹³), —OS(O)₁₋₂N(R¹²)(R¹³), —N(R¹¹)S(O)₁₋₂R¹¹, —NR¹¹S(O)₁₋₂OR¹¹, —NR¹¹S(O)₁₋₂N(R¹²)(R¹³), —P(O)(OR¹¹)₂, —OP(O)(OR¹¹)₂, —C(═X)R¹¹, —C(═X)XR¹¹, —XC(═X)R¹¹, and —XC(═X)XR¹¹, wherein each of the alkyl, alkenyl, alkynyl, cycloalkyl, aryl, heterocyclyl, and heteroaryl groups is optionally substituted with one or more independently selected R³⁰, wherein at least one of R⁷ is F and/or at least one of R⁷ is substituted with one or more F atoms

In a second additional (or alternative) embodiment of formula (I), when E is O and both of R^{4a} and R^{4b} are H, then R¹ and R³ join together via a group L′ to form a moiety R¹-L′-R³, preferably to form a moiety Q-L′-R³.

In one embodiment, the kinase inhibitor has the formula (Ia):

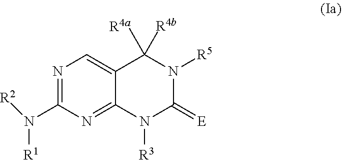

wherein R¹, R², R³, R^{4a}, R^{4b} and E are independently as defined above (in particular with respect to formula (I)) or below, and R⁵ is -L-R⁶, wherein L is as defined above (in particular with respect to formula (I) or (Ia)) or below, and R⁶ is a heteroaryl containing at least one ring heteroatom selected from the group consisting of N, O, and S, or is a heterocyclyl containing at least one ring heteroatom selected from the group consisting of N, O, and S, wherein each of the heteroaryl and heterocyclyl groups is optionally substituted with one or more, such as with one, two, or three, independently selected R⁷. For example, R⁶ may be a heteroaryl containing at least one ring heteroatom selected from the group consisting of N and O (i.e., the heteroaryl does not contain S as ring heteroatom; and in some embodiments does not contain O as ring heteroatom, i.e., R⁶ may by an N-heteroaryl), or is a heterocyclyl containing at least one ring heteroatom selected from the group consisting of N and O (i.e., the heterocyclyl does not contain S as ring heteroatom; and in some embodiments does not contain O as ring heteroatom, i.e., R⁶ may by an N-heterocyclyl), wherein each of the heteroaryl and heterocyclyl groups is optionally substituted with one or more, such as with one, two, or three, independently selected R⁷.

In one embodiment of the kinase inhibitor of formula (Ia), R¹, R², R³, R^{4a}, R^{4b}, R⁵ and E are independently as defined above (in particular with respect to formula (I) or (Ia)) or below, and R⁶ is 3- to 10-membered heteroaryl (e.g., containing at least one ring heteroatom selected from the group consisting of N, O, and S, such as from the group consisting of N and O) or a 3- to 10-membered heterocyclyl (e.g., containing at least one ring heteroatom selected from the group consisting of N, O, and S, such as from the group consisting of N and O), each of which is optionally substituted with one or more, such as with one, two, or three, independently selected R⁷.

In one embodiment of the kinase inhibitor of formula (Ia), R¹, R², R³, R^{4a}, R^{4b}, R⁵ and E are independently as defined above (in particular with respect to formula (I) or (Ia)) or below, and R⁶ is a mono- or bicyclic heteroaryl (e.g., containing at least one ring heteroatom selected from the group consisting of N, O, and S, such as from the group consisting of N and O) or a mono- or bicyclic heterocyclyl (e.g., containing at least one ring heteroatom selected from the group consisting of N, O, and S, such as from the group consisting of N and O), each of which is optionally substituted with one or more, such as with one, two, or three, independently selected R⁷.

In one embodiment of the kinase inhibitor of formula (Ia), R¹, R², R³, R^{4a}, R^{4b}, R⁵ and E are independently as defined above (in particular with respect to formula (I) or (Ia)) or below, and R⁶ is selected from the group consisting of a 5- to 6-membered monocyclic heteroaryl (e.g., containing at least one ring heteroatom selected from the group consisting of N, O, and S, such as from the group consisting of N and O), a 4- to 6-membered monocyclic heterocyclyl (e.g., containing at least one ring heteroatom selected from the group consisting of N, O, and S, such as from the group consisting of N and O), a 7- to 9-membered bicyclic heteroaryl (e.g., containing at least one ring heteroatom selected from the group consisting of N, O, and S, such as from the group consisting of N and O), and a 7- to 9-membered bicyclic heterocyclyl (e.g., containing at least one ring heteroatom selected from the group consisting of N, O, and S, such as from the group consisting of N and O), each of which is optionally substituted with one or more, such as with one, two, or three, independently selected R⁷.

In one embodiment of the kinase inhibitor of formula (Ia), R¹, R², R³, R^{4a}, R^{4b}, R⁵ and E are independently as defined above (in particular with respect to formula (I) or (Ia)) or below, and R⁶ is a 5- to 6-membered monocyclic heteroaryl (e.g., containing at least one ring heteroatom selected from the group consisting of N, O, and S, such as from the group consisting of N and O), which is optionally substituted with one or more, such as with one, two, or three, independently selected R⁷.

In one embodiment of the kinase inhibitor of formula (Ia), R¹, R², R³, R^{4a}, R^{4b}, R⁵ and E are independently as defined above (in particular with respect to formula (I) or (Ia)) or below, and R⁶ is a 7- to 9-membered bicyclic heterocyclyl (e.g., containing at least one ring heteroatom selected from the group consisting of N, O, and S, such as from the group consisting of N and O), which is optionally substituted with one or more, such as with one, two, or three, independently selected R⁷.

In one embodiment of the kinase inhibitor of formula (Ia), R¹, R², R³, R^{4a}, R^{4b}, R⁵ and E are independently as defined above (in particular with respect to formula (I) or (Ia)) or below, and R⁶ is selected from the group consisting of pyridinyl, thienyl, pyridazinyl, furanyl, pyrrolyl, pyrazolyl, oxazolyl, isoxazolyl, oxadiazolyl, imidazoimidazolyl, indolyl, naphthyridinyl, thienopyridinyl, tetrahydropyranyl, piperidinyl, pyrrolidinyl, azetidinyl, azabicycloheptanyl, azabicyclooctanyl, azapentacyclooctanyl, piperazinyl, morpholinyl, and tetrahydrothiophenyl, each of which is optionally substituted with one, two, or three independently selected R⁷, preferably R⁶ is selected from the group consisting of pyridinyl, thienyl, pyrazolyl, isoxazolyl, pyrrolyl, piperidinyl, pyrrolidinyl, azetidinyl, and azabicyclooctanyl, each of which is optionally substituted with one or more, such as with one, two, or three, independently selected R⁷.

In one embodiment of the kinase inhibitor of formula (Ia), R¹, R², R³, R^{4a}, R^{4b}, R⁵ and E are independently as defined above (in particular with respect to formula (I) or (Ia)) or below, and R⁷ may be independently selected from the group consisting of C₁₋₆ alkyl, C₂₋₆ alkenyl, C₂₋₆ alkynyl, halogen, —CN, —O(C₁₋₆ alkyl), —NH(C₁₋₆ alkyl), —N(C₁₋₆ alkyl)₂, —NHS(O)₁₋₂(C₁₋₆ alkyl), —NHS(O)₁₋₂O(C₁₋₆ alkyl), —C(═O)(C₁₋₆ alkyl), and —OC(═O)(C₁₋₆ alkyl), and/or any two R⁷ which are bound to the same atom of R⁶ being a heterocyclyl group may join together to form ═O, wherein each of the C₁₋₆ alkyl, C₂₋₆ alkenyl, and C₂₋₆ alkynyl groups is optionally substituted with one or more independently selected R³⁰. For example, R⁷ may be independently selected from the group consisting of C₁₋₃ alkyl, halogen, —CN, —O(C₁₋₃ alkyl), —NH(C₁₋₃ alkyl), and —N(C₁₋₃ alkyl)₂, and/or any two R⁷ which are bound to the same atom of R⁶ being a heterocyclyl group may join together to form ═O, wherein each of the C₁₋₃ alkyl groups is optionally substituted with one or more independently selected R³⁰. In any of the above embodiments of the kinase inhibitor of formula (V) (including those of formulas (Ia), (II), (III), (IV), (V), (VI), (VII), (VIIa), (VIII), (IX), (X), (XI), (XII)), R⁷ may be independently selected from the group consisting of Cl, Br, methyl, and ethyl, such as from the group consisting of Cl, Br, and methyl.

In one embodiment of the kinase inhibitor of formula (Ia), R¹, R², R³, R^{4a}, R^{4b}, R⁵ and E are independently as defined above (in particular with respect to formula (I) or (Ia)) or below, and where R⁶ is substituted, it is preferred that one R⁷ group is bound to a ring atom of R⁶ at position 2 relative to the ring atom by which R⁶ is bound to the remainder of the compound (i.e., it is preferred that R⁶ bears an ortho R⁷ group). In any of the embodiments disclosed herein of the kinase inhibitor of formula (I) (including those of formulas (Ia), (II), (III), (IV), (V), (Va), (VI), (VII), (VIIa), (VIII), (IX), (X), (XI), (XII)), where R⁶ is substituted with two or more (such as two, three, or four) R⁷ groups, it is preferred that one of the two or more R⁷ groups is bound to a ring atom of R⁶ at position 2 relative to the ring atom by which R⁶ is bound to the remainder of the compound (i.e., R⁶ bears an ortho R⁷ group), and the remaining R⁷ group(s) is (are) attached to ring atom(s) of R⁶ at positions other than position 2. E.g., in any of the above embodiments of the kinase inhibitor of formula (I) (including those of formulas (Ia), (II), (III), (IV), (V), (Va), (VI), (VII), (VIIa), (VIII), (IX), (X), (XI), (XII)), where R⁶ is an k-membered ring substituted with two or more R⁷ groups, it is preferred that one of the two or more R⁷ groups is bound to a ring atom of R⁶ at position 2 relative to the ring atom by which R⁶ is bound to the remainder of the compound (i.e., relative to the yl position) and that the remaining R⁷ group(s) is (are) bound to ring atoms of R⁶ at positions other than position 2, e.g., at position 3, 4, 5, . . . k. For example, in case R⁶ is a 5-membered ring, it is preferred that one of the two or more R⁷ groups is bound to a ring atom of R⁶ at position 2 (relative to the yl position) and that the remaining R⁷ group(s) is (are) bound to ring atoms of R⁶ at positions 3, 4, or 5 (relative to the yl position). Moreover, in any of the above embodiments of the kinase inhibitor of formula (I) (including those of formulas (Ia), (II), (III), (IV), (V), (Va), (VI), (VII), (VIIa), (VIII), (IX), (X), (XI), (XII)), where R⁶ is substituted with two or more (such as two, three, or four) R⁷ groups, it is preferred that each of the two ring atoms directly adjacent to the ring atom by which R⁶ is attached to the remainder of the compound bears one R⁷ group (e.g., R⁶ being an k-membered ring bears one R⁷ group at each of positions 2 and k, relative to the ring atom by which R⁶ is bound to the remainder of the compound, e.g., R⁶ is substituted at both of its ortho positions). Additionally, in any of the above embodiments of the kinase inhibitor of formula (I) (including those of formulas (Ia), (II), (III), (IV), (V), (Va), (VI), (VII), (VIIa), (VIII), (IX), (X), (XI), (XII)), where R⁶ is substituted with three or more (such as three or four) R⁷ groups, it is preferred that each of the two ring atoms directly adjacent to the ring atom by which R⁶ is attached to the remainder of the compound bears one R⁷ group (e.g., R⁶ being an k-membered ring bears one R⁷ group at each of positions 2 and k, relative to the ring atom by which R⁶ is bound to the remainder of the compound, e.g., R⁶ is substituted at both of its ortho positions), and that the third R⁷ group is bound to a ring atom of R⁶ which is directly adjacent to one of the ortho ring atoms but which is not the ring atom by which R⁶ is bound to the remainder of the compound (e.g., R⁶ being an k-membered ring bears the third R⁷ group at one of positions 3 and k-1, relative to the ring atom by which R⁶ is bound to the remainder of the compound).

In one embodiment of the kinase inhibitor of formula (Ia), R¹, R², R³, R^{4a}, R^{4b}, R⁵ and E are independently as defined above (in particular with respect to formula (I) or (Ia)) or below, and and R⁶ is selected from the following formulas:

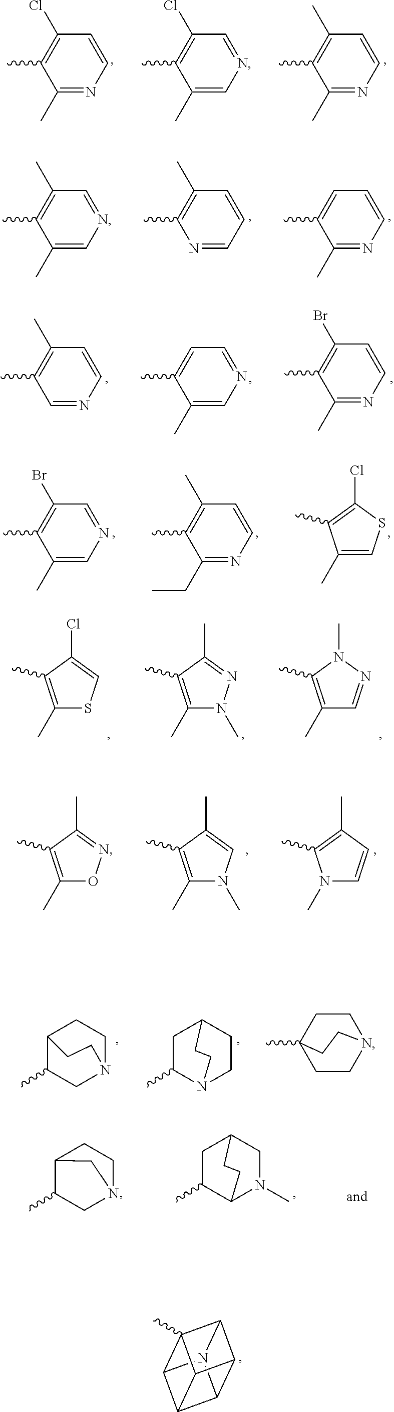

wherein [symbol] represents the bond by which R⁶ is bound to the remainder of the compound.

In one embodiment of the kinase inhibitor of formula (Ia), R¹, R², R³, R^{4a}, R^{4b}, R⁵ and E are independently as defined above (in particular with respect to formula (I) or (Ia)) or below, and R⁶ is selected from the following formulas:

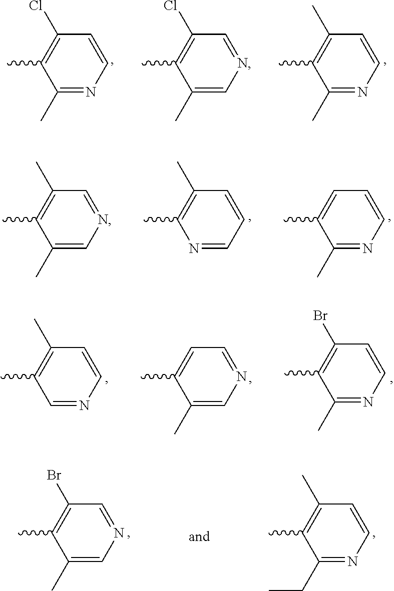

wherein [symbol] represents the bond by which R⁶ is bound to the remainder of the compound.

In one embodiment of the kinase inhibitor of formula (Ia), R¹, R², R³, R^{4a}, R^{4b}, R⁵ and E are independently as defined above (in particular with respect to formula (I) or (Ia)) or below, and R⁶ is selected from the following formulas:

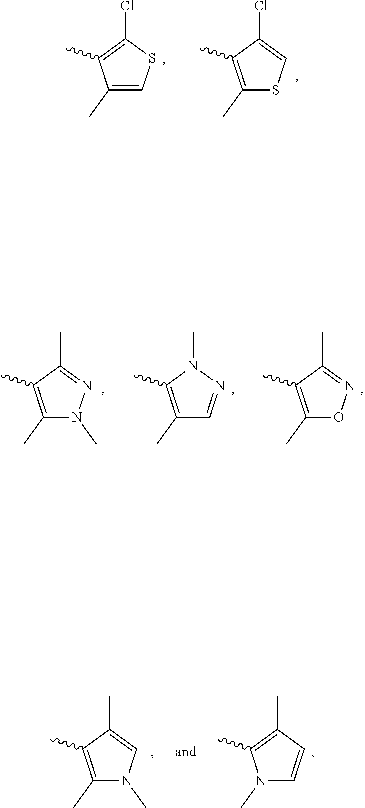

wherein [symbol] represents the bond by which R⁶ is bound to the remainder of the compound.

In one embodiment of the kinase inhibitor of formula (Ia), R¹, R², R³, R^{4a}, R^{4b}, R⁵ and E are independently as defined above (in particular with respect to formula (I) or (Ia)) or below, and R⁶ is selected from the following formulas:

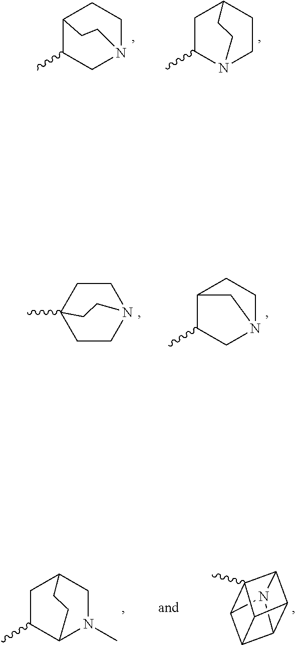

wherein [symbol] represents the bond by which R⁶ is bound to the remainder of the compound.

In one embodiment, the kinase inhibitor has the formula (II):

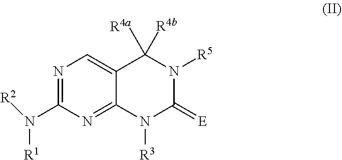

wherein R¹, R², R³, R^{4a}, R^{4b} and E are independently as defined above (in particular with respect to formula (I) or (Ia)) or below, and R⁵ is -L-R⁶, wherein L is as defined above (in particular with respect to formula (I) or (Ia)) or below and R⁶ is a 5-membered monocyclic heteroaryl which contains at least one S ring atom and which is substituted with one or more independently selected R⁷;

optionally, wherein R⁷ is independently selected from the group consisting of alkyl, alkenyl, alkynyl, cycloalkyl, aryl, heterocyclyl, heteroaryl, halogen, —CN, azido, —NO₂, —OR¹¹, —N(R¹²)(R¹³), —N(R¹¹)(OR¹¹), —S(O)₀₋₂R¹¹, —S(O)₁₋₂OR¹¹, —OS(O)₁₋₂R¹¹, —OS(O)₁₋₂OR¹¹, —S(O)₁₋₂N(R¹²)(R¹³), —OS(O)₁₋₂N(R¹²)(R¹³), —N(R¹¹)S(O)₁₋₂R¹¹, —NR¹¹S(O)₁₋₂OR¹¹, —NR¹¹S(O)₁₋₂N(R¹²)(R¹³), —P(O)(OR¹¹)₂, —OP(O)(OR¹¹)₂, —C(═X)R¹¹, —C(═X)XR¹¹, —XC(═X)R¹¹, and —XC(═X)XR¹¹; and wherein each of the alkyl, alkenyl, alkynyl, cycloalkyl, aryl, heterocyclyl, and heteroaryl groups is optionally substituted with one or more independently selected R³⁰; and optionally, wherein at least one of R⁷ is F and/or at least one of R⁷ is substituted with one or more F atoms.

In one embodiment of the kinase inhibitor of formula (I), (Ia) or (II), R¹, R², R³, R^{4a}, R^{4b}, R⁵ and E are independently as defined above (in particular with respect to formula (I), (Ia) or (II)) or below, and at least one of R⁷ is F and/or at least one of R⁷ is selected from the group consisting of alkyl, —OR¹¹, and —N(R¹²)(R¹³), wherein each of the alkyl and R¹¹ groups and at least one of the R¹² and R¹³ groups is substituted with one or more F atoms. In a first example of such embodiments, at least one of R⁷ is F and/or at least one of R⁷ is selected from the group consisting of alkyl, —O(alkyl), —NH(alkyl), and —N(alkyl)₂, wherein the alkyl group of alkyl, —O(alkyl) and —NH(alkyl) and at least one of the alkyl groups of —N(alkyl)₂ is substituted with one or more F atoms. In a second example of such embodiments, at least one of R⁷ is F and/or at least one of R⁷ is selected from the group consisting of C₁₋₃alkyl, —O(C₁₋₃alkyl), —NH(C₁₋₃alkyl) or —N(C₁₋₃alkyl)₂, wherein the alkyl group of C₁₋₃alkyl, —NH(C₁₋₃alkyl), and —O(C₁₋₃alkyl) and at least one of the alkyl groups of —N(C₁₋₃alkyl)₂ is substituted with one or more F atoms. In a third example of such embodiments, at least one of R⁷ is F and/or at least one of R⁷ is C₁₋₃ alkyl, wherein the alkyl group of C₁₋₃alkyl is substituted with one or more F atoms.

In one embodiment of the kinase inhibitor of formula (I) or (Ia), R¹, R², R³, R^{4a}, R^{4b}, R⁵ and E are independently as defined above (in particular with respect to formula (I), (Ia) or (II)) or below, and R⁶ is a mono- or bicyclic heteroaryl or a mono- or bicyclic heterocyclyl, each of which is optionally substituted with one or more independently selected R⁷.

In one embodiment of the kinase inhibitor of formula (I) or (Ia)), R¹, R², R³, R^{4a}, R^{4b}, R⁵ and E are independently as defined above (in particular with respect to formula (I), (Ia) or (II)) or below, and R⁶ is a 5- to 6-membered monocyclic heteroaryl optionally substituted with one, two, three or four independently selected R⁷.

In one embodiment of the kinase inhibitor of formula (I) or (Ia), R¹, R², R³, R^{4a}, R^{4b}, R⁵ and E are independently as defined above (in particular with respect to formula (I), (Ia) or (II)) or below, and R⁶ is a 5-membered monocyclic heteroaryl which contains at least one ring heteroatom selected from the group consisting of N, O, and S, preferably thionyl, isoxazoyl and pyrazolyl, and which is optionally substituted with one, two, or three independently selected R⁷.

In one embodiment of the kinase inhibitor of formula (I) or (Ia), R¹, R², R³, R^{4a}, R^{4b}, R⁵ and E are independently as defined above (in particular with respect to formula (I), (Ia) or (II)) or below, and R⁶ is a 5- or 6-membered monocyclic heteroaryl which contains at least one S ring atom and which is optionally substituted with one, two, or three independently selected R7.

In one embodiment of the kinase inhibitor of formula (I), (Ia) or (II), R¹, R², R³, R^{4a}, R^{4b}, R⁵ and E are independently as defined above (in particular with respect to formula (I), (Ia) or (II)) or below, and R⁶ is thienyl optionally substituted with one, two, or three independently selected R⁷.

In one embodiment of the kinase inhibitor of formula (I), (Ia) or (II), R¹, R², R³, R^{4a}, R^{4b}, R⁵ and E are independently as defined above (in particular with respect to formula (I), (Ia) or (II)) or below, and R⁶ is substituted with one or more, such as with one, two, or three, independently selected R⁷. In particular of such embodiments, R⁶ is substituted with two independently selected R⁷, optionally wherein R⁶ is substituted with two R⁷ that differ from each other.

In one embodiment of the kinase inhibitor of formula (I), (Ia) or (II), R¹, R², R³, R^{4a}, R^{4b}, R⁵ and E are independently as defined above (in particular with respect to formula (I), (Ia) or (II)) or below, and R⁷ is independently selected from the group consisting of C₁₋₃ alkyl, halogen, —CN, —O(C₁₋₃ alkyl), —NH(C₁₋₃ alkyl), and —N(C₁₋₃ alkyl)₂, and/or any two R⁷ which are bound to the same atom of R⁶ being a heterocyclyl group may join together to form ═O, wherein each of the C₁₋₃ alkyl groups is optionally substituted with one or more independently selected R³⁰. In particular of such embodiments, R⁷ is independently selected from the group consisting of C₁₋₃ alkyl, halogen, —CN, —O(C₁₋₃ alkyl), —NH(C₁₋₃ alkyl), and —N(C₁₋₃ alkyl)₂, wherein each of the C₁₋₃ alkyl groups is optionally substituted with one or more independently selected R³⁰.

In one embodiment of the kinase inhibitor of formula (I), (Ia) or (II), R¹, R², R³, R^{4a}, R^{4b}, R⁵ and E are independently as defined above (in particular with respect to formula (I). (Ia) or (II)) or below, and R⁷ is independently selected from the group consisting of halogen, preferably F, Cl, or Br, and C₁₋₂ alkyl, wherein the C₁₋₂ alkyl groups is optionally substituted with one, two, or three independently selected R³⁰.

In one embodiment of the kinase inhibitor of formula (I), (Ia) or (II), R¹, R², R³, R^{4a}, R^{4b}, R⁵ and E are independently as defined above (in particular with respect to formula (I), (Ia) or (II)) or below, and R⁷ is independently selected from the group consisting of Cl, F, methyl, fluoromethyl, difluoromethyl, and trifluoromethyl.

In one embodiment of the kinase inhibitor of formula (I), (Ia) or (II), R¹, R², R³, R^{4a}, R^{4b}, R⁵ and E are independently as defined above (in particular with respect to formula (I), (Ia) or (II)) or below, and optionally, when R⁶ is a 5- to 6-membered monocyclic heteroaryl (e.g., containing at least one ring heteroatom selected from the group consisting of N, O, and S, such as from the group consisting of N and O), then one R⁷ group is bound to a ring atom of R⁶ at position 2 relative to the ring atom by which R⁶ is bound to the remainder of the compound. In certain of such embodiments where R⁶ is a 5-membered monocyclic heteroaryl which contains at least one S ring atom, then one R⁷ is attached to the C ring atom at position 2 relative to the ring atom by which R⁶ is bound to the remainder of the compound.

In one embodiment of the kinase inhibitor of formula (I), (Ia) or (II), R¹, R², R³, R^{4a}, R^{4b}, R⁵ and E are independently as defined above (in particular with respect to formula (I), (Ia) or (II)) or below, and optionally when R⁶ is a 5- to 6-membered (preferably, a 5-membered) monocyclic heteroaryl (e.g., containing at least one ring heteroatom selected from the group consisting of N, O, and S, such as from the group consisting of N and O) such as when R⁶ is (i) (i) a 5-membered monocyclic heteroaryl which contains at least one S ring atom and which is substituted with one or more independently selected R⁷; (ii) a 5-membered monocyclic heteroaryl which contains at least two nitrogen atoms and which is substituted with one or more independently selected R⁷; or (iii) a 5-membered monocyclic heteroaryl which contains at least one nitrogen atom and at least one oxygen atom and which is substituted with one or more independently selected R⁷, then one R⁷ group is bound to a ring atom of R⁶ at position 5 relative to the ring atom by which R⁶ is bound to the remainder of the compound. In certain of such embodiments where R⁶ is a 5-membered monocyclic heteroaryl which contains at least one S ring atom, then one R⁷ is attached to the C ring atom at position 5 relative to the ring atom by which R⁶ is bound to the remainder of the compound.

In one embodiment of the kinase inhibitor of formula (I), (Ia) or (II), R¹, R², R³, R^{4a}, R^{4b}, R⁵ and E are independently as defined above (in particular with respect to formula (I), (Ia) or (II)) or below, and R⁶ is substituted with two, or three independently selected R⁷, and optionally when R⁶ is a 5- to 6-membered (preferably, a 5-membered) monocyclic heteroaryl (e.g., containing at least one ring heteroatom selected from the group consisting of N, O, and S, such as from the group consisting of N and O) such as when R⁶ is (i) (i) a 5-membered monocyclic heteroaryl which contains at least one S ring atom and which is substituted with one or more independently selected R⁷; (ii) a 5-membered monocyclic heteroaryl which contains at least two nitrogen atoms and which is substituted with one or more independently selected R⁷; or (iii) a 5-membered monocyclic heteroaryl which contains at least one nitrogen atom and at least one oxygen atom and which is substituted with one or more independently selected R⁷, then one R⁷ group is bound to a ring atom of R⁶ at position 2 relative to the ring atom by which R⁶ is bound to the remainder of the compound, and another R⁷ group is bound to a ring atom of R⁶ at position 5 relative to the ring atom by which R⁶ is bound to the remainder of the compound. In certain of such embodiments where R⁶ is a 5-membered monocyclic heteroaryl which contains at least one S ring atom, then one R⁷ is attached to the C ring atom at position 5 relative to the ring atom by which R⁶ is bound to the remainder of the compound, then one R⁷ is attached to the C ring atom at position 2 relative to the ring atom by which R⁶ is bound to the remainder of the compound and one R⁷ is attached to the C ring atom at position 5 relative to the ring atom by which R⁶ is bound to the remainder of the compound.

In one embodiment of the kinase inhibitor of formula (I), (Ia) or (II), R¹, R², R³, R^{4a}, R^{4b}, R⁵ and E are independently as defined above (in particular with respect to formula (I), (Ia) or (II)) or below, and at least one of R⁷ is F and/or at least one of R⁷ is selected from the group consisting of alkyl, —O(alkyl), —NH(alkyl), and —N(alkyl)₂, wherein the alkyl group of alkyl, —O(alkyl) and —NH(alkyl) and at least one of the alkyl groups of —N(alkyl)₂ is substituted with one or more F atoms.

In one embodiment of the kinase inhibitor of formula (I), (Ia) or (II), R¹, R², R³, R^{4a}, R^{4b}, R⁵ and E are independently as defined above (in particular with respect to formula (I), (IA) or (II)) or below, and at least one of R⁷ is F and/or at least one of R⁷ is selected from the group consisting of C₁₋₃alkyl, —O(C₁₋₃alkyl), —NH(C₁₋₃alkyl) or —N(C₁₋₃alkyl)₂, wherein the alkyl group of C₁₋₃ alkyl, —NH(C₁₋₃alkyl), and —O(C₁₋₃alkyl) and at least one of the alkyl groups of —N(C₁₋₃alkyl)₂ is substituted with one or more F atoms.

In one embodiment of the kinase inhibitor of formula (I), (Ia) or (II), R¹, R², R³, R^{4a}, R^{4b}, R⁵ and E are independently as defined above (in particular with respect to formula (I), (Ia) or (II)) or below, and at least one of R⁷ is F and/or at least one of R⁷ is C₁₋₃ alkyl, wherein the alkyl group of C₁₋₃ alkyl is substituted with one or more F atoms.

In one embodiment of the kinase inhibitor of formula (I), (Ia) or (II), R¹, R², R³, R^{4a}, R^{4b}, R⁵ and E are independently as defined above (in particular with respect to formula (I), (Ia) or (II)) or below, and at least one of R⁷ is F and/or at least one of R⁷ is selected from the group consisting of —CH₂F, —CHF₂, and —CF₃, preferably selected from the group consisting of —CH₂F and —CHF₂.

In one embodiment of the kinase inhibitor of formula (I), (Ia) or (II), R¹, R², R³, R^{4a}, R^{4b}, R⁵ and E are independently as defined above (in particular with respect to formula (I), (Ia) or (II)) or below, and optionally when R⁶ is a 5-membered monocyclic heteroaryl preferably which contains at least one S ring atom, then one R⁷ is attached to the C ring atom at position 2 relative to the ring atom by which R⁶ is bound to the remainder of the compound, preferably wherein said R⁷ is F and/or said R⁷ is substituted with one or more F atoms.

In one embodiment of the kinase inhibitor of formula (I), (Ia) or (II), R¹, R², R³, R^{4a}, R^{4b}, R⁵ and E are independently as defined above (in particular with respect to formula (I), (Ia) or (II)) or below, and optionally when R⁶ is a 5-membered monocyclic heteroaryl preferably which contains at least one S ring atom, then one R⁷ is attached to the C ring atom at position 5 relative to the ring atom by which R⁶ is bound to the remainder of the compound, preferably wherein said R⁷ is F and/or said R⁷ is substituted with one or more F atoms.

In one embodiment of the kinase inhibitor of formula (I), (Ia) or (II), R¹, R², R³, R^{4a}, R^{4b}, R⁵ and E are independently as defined above (in particular with respect to formula (I), (Ia) or (II)) or below, and R⁶ is selected from the group consisting of

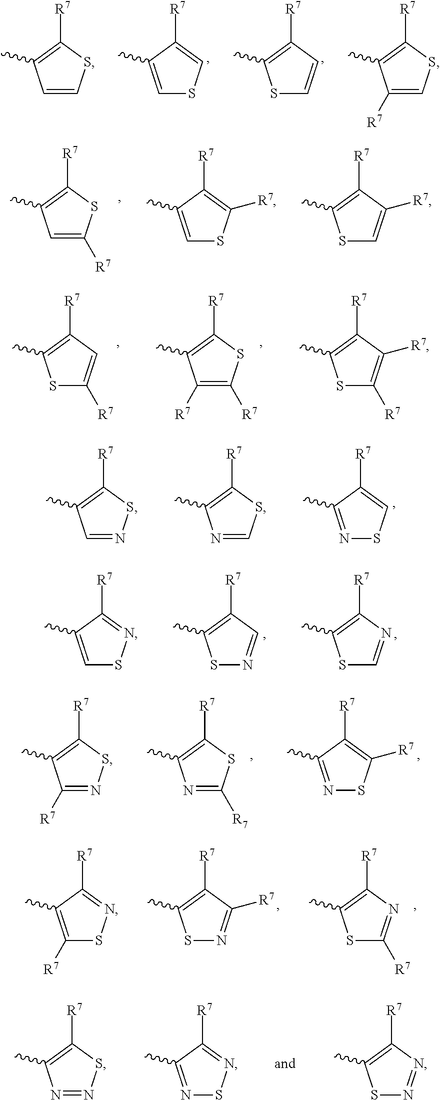

wherein [symbol] represents the bond by which R⁶ is bound to the remainder of the compound.

In another embodiment of the kinase inhibitor of formula (I), (Ia) or (II), R¹, R², R³, R^{4a}, R^{4b}, R⁵ and E are independently as defined above (in particular with respect to formula (I), (Ia) or (II)) or below, and R⁶ is selected from the group consisting of

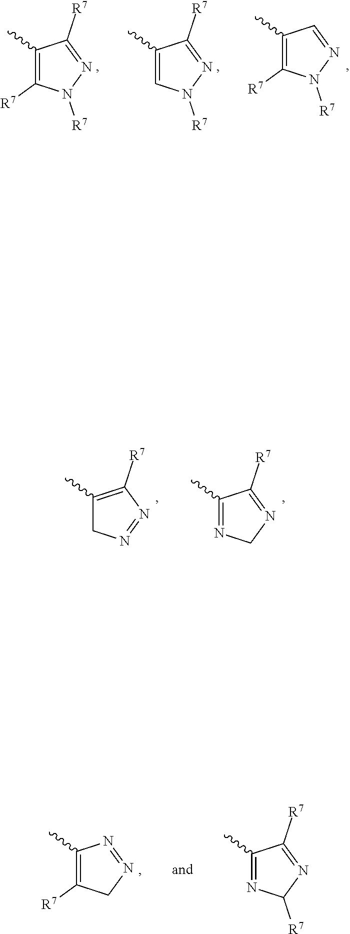

wherein [symbol] represents the bond by which R⁶ is bound to the remainder of the compound.

In yet another embodiment of the kinase inhibitor of formula (I), (Ia) or (II), R¹, R², R³, R^{4a}, R^{4b}, R⁵ and E are independently as defined above (in particular with respect to formula (I), (Ia) or (II)) or below, and R⁶ is selected from the group consisting of

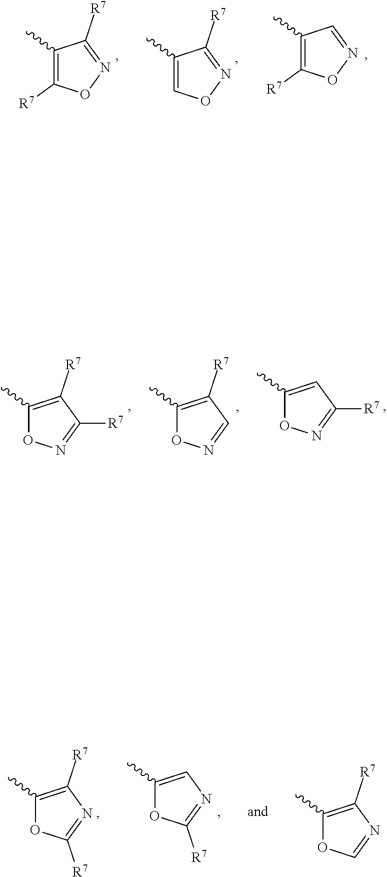

wherein [symbol] represents the bond by which R⁶ is bound to the remainder of the compound.

In one embodiment of the kinase inhibitor of formula (I), (Ia) or (II), R¹, R², R³, R^{4a}, R^{4b}, R⁵ and E are independently as defined above (in particular with respect to formula (I), (Ia) or (II)) or below, and R⁶ is substituted with at least two R⁷.

In one embodiment of the kinase inhibitor of formula (I), (Ia) or (II), R¹, R², R³, R^{4a}, R^{4b}, R⁵ and E are independently as defined above (in particular with respect to formula (I), (Ia) or (II)) or below, and R⁶ is substituted with two R⁷ which differ from each other.

In one embodiment of the kinase inhibitor of formula (I), (Ia) or (II), R¹, R², R³, R^{4a}, R^{4b}, R⁵ and E are independently as defined above (in particular with respect to formula (I), (Ia) or (II)) or below, and optionally when R⁶ is a 5-membered monocyclic heteroaryl preferably which contains at least one S ring atom, then one R⁷ is attached to the C ring atom at position 2 relative to the ring atom by which R⁶ is bound to the remainder of the compound and one R⁷ is attached to the C ring atom at position 5 relative to the ring atom by which R⁶ is bound to the remainder of the compound, preferably wherein at least one of said R⁷ is F and/or at least one of R⁷ is substituted with one or more F atoms.

In one embodiment of the kinase inhibitor of formula (I), (Ia) or (II), R¹, R², R³, R^{4a}, R^{4b}, R⁵ and E are independently as defined above (in particular with respect to formula (I), (Ia) or (II)) or below, and wherein one R⁷ is selected from the group consisting of —CH₂F, —CHF₂, and —CF₃, and one R⁷ is selected from the group consisting of halogen, —CH₃, —CH₂(hal), —CH(hal)₂, and —C(hal)₃, more preferably selected from the group consisting of Cl, Br, F, CH₃, —CH₂F, —CHF₂, and —CF₃.

In one embodiment of the kinase inhibitor of formula (I), (Ia) or (II), R¹, R², R³, R^{4a}, R^{4b}, R⁵ and E are independently as defined above (in particular with respect to formula (I), (Ia) or (II)) or below, and one R⁷ is selected from the group consisting of —CH₂F, —CHF₂, and —CF₃, preferably selected from the group consisting of —CH₂F and —CHF₂, and one R⁷ is Cl.

In one embodiment of the kinase inhibitor of formula (I), (Ia) or (II), R¹, R², R³, R^{4a}, R^{4b}, R⁵ and E are independently as defined above (in particular with respect to formula (I), (Ia) or (II)) or below, and one R⁷ is F, and one R⁷ is selected from the group consisting of halogen, CH₃, —CH₂(hal), —CH(hal)₂, and —C(hal)₃, more preferably selected from the group consisting of Cl, Br, F, CH₃, —CH₂F, —CHF₂, and —CF₃.

In one embodiment of the kinase inhibitor of formula (I), (Ia) or (II), R¹, R², R³, R^{4a}, R^{4b}, R⁵ and E are independently as defined above (in particular with respect to formula (I), (Ia) or (II)) or below, and one R⁷ is F and one R⁷ is Cl.

In one embodiment of the kinase inhibitor of formula (I), (Ia) or (II), R¹, R², R³, R^{4a}, R^{4b}, R⁵ and E are independently as defined above (in particular with respect to formula (I), (Ia) or (II)) or below, and R⁶ is selected from the group consisting of: (i) thienyl, thiazolyl, and thiadiazolyl; (ii) pyrazolyl and imidazolyl; and/or (iii) oxazolyl, oxadiazolyl, and isoxazolyl, each of which is substituted with one or more independently selected R⁷. In one of such embodiments, R⁶ is selected from the group consisting of: (i) thienyl, thiazolyl, and thiadiazolyl.

In one embodiment of the kinase inhibitor of formula (I), (Ia) or (II), R¹, R², R³, R^{4a}, R^{4b}, R⁵ and E are independently as defined above (in particular with respect to formula (I), (Ia) or (II)) or below, and R⁶ is selected from the group consisting of thienyl and thiazolyl, each of which is substituted with one or more independently selected R⁷.

In one embodiment of the kinase inhibitor of formula (I), (Ia) or (II), R¹, R², R³, R^{4a}, R^{4b}, R⁵ and E are independently as defined above (in particular with respect to formula (I), (Ia) or (II)) or below, and the ring atom of R⁶ by which R⁶ is bound to the remainder of the compound is a C atom.

In one embodiment of the kinase inhibitor of formula (I), (Ia) or (II), R¹, R², R³, R^{4a}, R^{4b}, R⁵ and E are independently as defined above (in particular with respect to formula (I), (Ia) or (II)) or below, and R⁶ is a 5-membered monocyclic heteroaryl which contains at least one S ring atom, then the S ring atom of R⁶ is not adjacent to the ring atom by which R⁶ is bound to the remainder of the compound.

In one embodiment of the kinase inhibitor of formula (I), (Ia) or (II), R¹, R², R³, R^{4a}, R^{4b}, R⁵ and E are independently as defined above (in particular with respect to formula (I), (Ia) or (II)) or below, and R⁶ is selected from the group consisting of

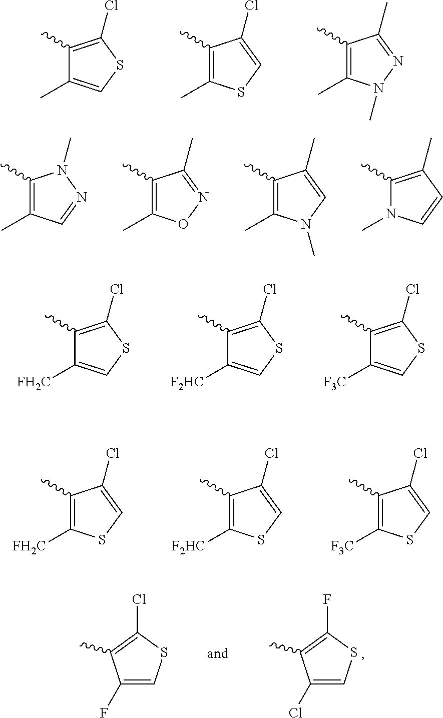

wherein [symbol] represents the bond by which R⁶ is bound to the remainder of the compound.

In one embodiment of the kinase inhibitor of formula (I), (Ia) or (II), R¹, R², R³, R^{4a}, R^{4b}, R⁵ and E are independently as defined above (in particular with respect to formula (I), (Ia) or (II)) or below, and R⁶ is selected from the group consisting of

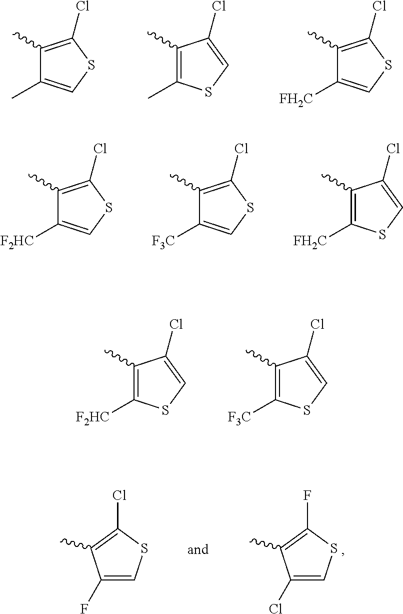

wherein [symbol] represents the bond by which R⁶ is bound to the remainder of the compound.

In one embodiment of the kinase inhibitor of formula (I), (Ia) or (II), R¹, R², R³, R^{4a}, R^{4b}, R⁵ and E are independently as defined above (in particular with respect to formula (I), (Ia) or (II)) or below, and R⁶ is selected from the group consisting of

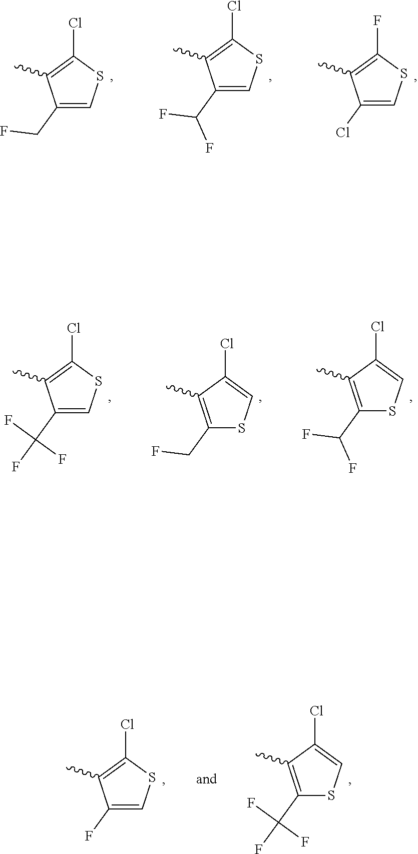

wherein [symbol] represents the bond by which R⁶ is bound to the remainder of the compound.

In one embodiment of the kinase inhibitor of formula (I), (Ia) or (II), R¹, R², R³, R^{4a}, R^{4b}, R⁵ and E are independently as defined above (in particular with respect to formula (I), (Ia) or (II)) or below, and R⁶ is selected from the group consisting of

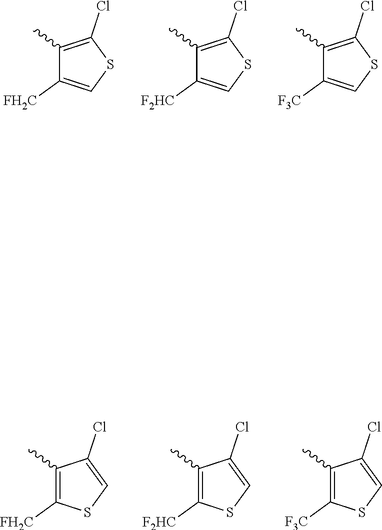

wherein [symbol] represents the bond by which R⁶ is bound to the remainder of the compound.

In one embodiment of the kinase inhibitor of formula (I), (Ia) or (II), R¹, R², R³, R^{4a}, R^{4b}, R⁵ and E are independently as defined above (in particular with respect to formula (I), (Ia) or (II)) or below, and R⁶ is selected from the group consisting of

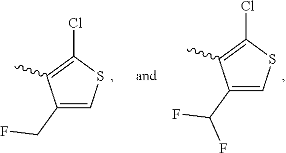

wherein [symbol] represents the bond by which R⁶ is bound to the remainder of the compound.

[symbol]

In another embodiment of the kinase inhibitor of formula (I), (Ia) or (II), R¹, R², R³, R^{4a}, R^{4b}, R⁵ and E are independently as defined above (in particular with respect to formula (I), (Ia) or (II)) or below, and R⁶ is selected from the group consisting of

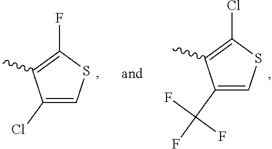

wherein [symbol] represents the bond by which R⁶ is bound to the remainder of the compound.

In a first alternative embodiment of the kinase inhibitor of formula (I), (Ia) or (II), R¹, R², R³, R^{4a}, R^{4b}, R⁵ and E are independently as defined above (in particular with respectto formula (I), (Ia) or (II)) or below, and R⁶ is selected from the group consisting of thienyl, thiazolyl, and thiadiazolyl, each of which is substituted with one or more (such as 1 to the maximum number of hydrogen atoms bound to the 5-membered monocyclic heteroaryl group, e.g., 1, 2, or 3) independently selected R⁷. For example, R⁶ may be selected from the group consisting of thienyl and thiazolyl, each of which is substituted with one or more (such as 1 to the maximum number of hydrogen atoms bound to the 5-membered monocyclic heteroaryl group, e.g., 1, 2, or 3) independently selected R⁷. Preferably, R⁶ is thienyl which is substituted with one or more (such as 1 to the maximum number of hydrogen atoms bound to the 5-membered monocyclic heteroaryl group, e.g., 1, 2, or 3, preferably 2) independently selected R⁷.

In a second alternative embodiment of the kinase inhibitor of formula (I), (Ia) or (II), R¹, R², R³, R^{4a}, R^{4b}, R⁵ and E are independently as defined above (in particular with respect to formula (I), (Ia) or (II)) or below, and R⁶ is selected from the group consisting of pyrazolyl and imidazolyl, each of which is substituted with one or more (such as 1 to the maximum number of hydrogen atoms bound to the 5-membered monocyclic heteroaryl group, e.g., 1, 2, or 3) independently selected R⁷. For example, R⁶ may be imidazolyl which is substituted with one or more (such as 1 to the maximum number of hydrogen atoms bound to the 5-membered monocyclic heteroaryl group, e.g., 1, 2, or 3) independently selected R⁷. In a preferable of such embodiment, R⁶ is pyrazolyl which is substituted with one or more (such as 1 to the maximum number of hydrogen atoms bound to the 5-membered monocyclic heteroaryl group, e.g., 1, 2, or 3, preferably 2) independently selected R⁷.

In a third alternative embodiment of the kinase inhibitor of formula (I), (Ia) or (II), R¹, R², R³, R^{4a}, R^{4b}, R⁵ and E are independently as defined above (in particular with respect to formula (I), (Ia) or (II)) or below, and R⁶ is selected from the group consisting of oxazolyl, oxadiazolyl, and isoxazolyl, each of which is substituted with one or more (such as 1 to the maximum number of hydrogen atoms bound to the 5-membered monocyclic heteroaryl group, e.g., 1, 2, or 3) independently selected R⁷. For example, R⁶ may be selected from the group consisting of oxazolyl and oxadiazolyl, each of which is substituted with one or more (such as 1 to the maximum number of hydrogen atoms bound to the 5-membered monocyclic heteroaryl group, e.g., 1, 2, or 3) independently selected R⁷. Preferably, R⁶ is oxazolyl which is substituted with one or more (such as 1 to the maximum number of hydrogen atoms bound to the 5-membered monocyclic heteroaryl group, e.g., 1, 2, or 3, preferably 2) independently selected R⁷.

In one embodiment, the kinase inhibitor has the formula (III):

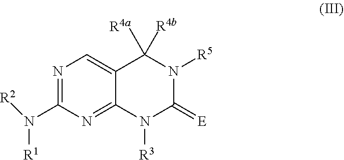

wherein R¹, R², R³, R^{4a}, R^{4b}, R⁵ and E are independently as defined above (in particular with respect to formula (I), (Ia) or (II)) or below, and R^{1a} of R¹ is selected from the group consisting of alkyl, —O(alkyl), —S(alkyl), —NH(alkyl), —N(alkyl)₂, and heterocyclyl, wherein each of the alkyl and heterocyclyl groups is optionally substituted with one or more (e.g., one, two, or three) independently selected R³⁰. Preferably, each R³⁰ is independently a 1st level substituent, a 2^{nd} level substituent, or a 3^{rd} level substituent as specified herein, such as alkyl (e.g., C₁₋₆ alkyl), —(CH₂)₁₋₃OH, alkenyl (e.g., C₂₋₆ alkenyl), alkynyl (e.g., C₂₋₆ alkynyl), halogen, —CN, nitro, —OR¹¹ (e.g., —OH), —SR¹¹ (e.g., —SH), —N(R¹²)(R¹³) (e.g., —NH₂), and —C(═O)R¹¹ (e.g., —C(═O)(C₁₋₃ alkyl)).

In one embodiment of the kinase inhibitor of formula (III), R¹, R², R³, R^{4a}, R^{4b}, R⁵ and E are independently as defined above (in particular with respect to formula (I), (Ia) or (II)) or below, and R^{1a} is selected from the group consisting of alkyl, —O(alkyl), —S(alkyl), —NH(alkyl), —N(alkyl)₂, and heterocyclyl, wherein each of the alkyl and heterocyclyl groups is optionally substituted with one or more independently selected R³⁰.

In one embodiment of the kinase inhibitor of formula (III), R¹, R², R³, R^{4a}, R^{4b}, R⁵ and E are independently as defined above (in particular with respect to formula (I), (Ia) or (II)) or below, and R³⁰ optionally substituting R^{1a} are selected from (i) C₁₋₃ alkyl, phenyl, thiazolidinyl, halogen, —NH₂, —NHS(O)₂(C₁₋₃ alkyl), —NHC(═O)(C₁₋₃ alkyl), and —NHC(═NH)NH_{z-2}(C₁₋₃ alkyl)_{z}, wherein z is 0, 1, or 2 and each C₁₋₃ alkyl is independently methyl, ethyl, propyl or isopropyl; (ii) methyl, ethyl, propyl, isopropyl, phenyl, ═O, and ═S; or (iii) methyl, ethyl, propyl, isopropyl, halogen, and —CF₃.

In one embodiment of the kinase inhibitor of formula (III), R¹, R², R³, R^{4a}, R^{4b}, R⁵ and E are independently as defined above (in particular with respect to formula (I), (Ia) or (II)) or below, and R^{1a} is selected from the group consisting of —O(alkyl), —S(alkyl), —NH(alkyl), —N(alkyl)₂, and heterocyclyl, wherein each of the alkyl and heterocyclyl groups is optionally substituted with one or more independently selected R³⁰.

In one embodiment of the kinase inhibitor of formula (III), R¹, R², R³, R^{4a}, R^{4b}, R⁵ and E are independently as defined above (in particular with respect to formula (I), (Ia) or (II)) or below, and the one or more independently selected R³⁰ optionally substituting R^{1a} are selected from (i) C₁₋₃ alkyl, phenyl, thiazolidinyl, halogen, —NH₂, —NHS(O)₂ (C₁₋₃ alkyl), —NHC(═O)(C₁₋₃ alkyl), and —NHC(═NH)NH_{z-2}(C₁₋₃ alkyl)_{z}, wherein z is 0, 1, or 2 and each C₁₋₃ alkyl is independently methyl, ethyl, propyl or isopropyl; (ii) methyl, ethyl, propyl, isopropyl, phenyl, ═O, and ═S; or (iii) methyl, ethyl, propyl, isopropyl, halogen, and —CF₃.

In one embodiment of the kinase inhibitor of formula (III), R¹, R², R³, R^{4a}, R^{4b}, R⁵ and E are independently as defined above (in particular with respect to formula (I), (Ia) or (II)) or below, and R^{1a} is selected from the group consisting of C₁₋₃ alkyl, —O(C₁₋₃ alkyl), —S(C₁₋₃ alkyl), —NH(C₁₋₃ alkyl), —N(C₁₋₃ alkyl)₂, and 3- to 7-membered heterocyclyl (preferably, a 3- to 7-membered heterocyclyl), wherein the 3- to 7-membered heterocyclyl group is optionally substituted with one or two moieties independently selected from the group consisting of methyl, ethyl, —OH, —OCH₃, —SCH₃, cyclopropyl, 2-hydroxyethyl, 2-(N,N-dimethylamino)ethyl, 2-(N,N-dimethylamino)ethoxy, 2-aminoethyl, 2-(N-methylamino)ethyl, 2-(methoxy)ethyl, 4-methylpiperazinyl, —C(═O)(c₁₋₃ alkyl), —(CH₂)₁₋₃COOH, and —NH_{2-z}(CH₃)_{z}, wherein z is 0, 1, or 2; and each of the C₁₋₃ alkyl groups is optionally substituted with one or two moieties independently selected from the group consisting of —OH, —OCH₃, —SCH₃, cyclopropyl, piperazinyl, 4-methyl-piperazinyl, 4-(2-hydroxyethyl)piperazinyl, 2-(N,N-dimethylamino)ethoxy, and —NH_{2-z}(CH₃)_{z}, wherein z is 0, 1, or 2.

In one embodiment of the kinase inhibitor of formula (III), R¹, R², R³, R^{4a}, R^{4b}, R⁵ and E are independently as defined above (in particular with respect to formula (I), (Ia) or (II)) or below, and R^{1a} is selected from the group consisting of C₁₋₃ alkyl, —O(C₁₋₃ alkyl), —S(C₁₋₃ alkyl), —NH(C₁₋₃ alkyl), piperazinyl, piperidinyl, hexahydropyrimidinyl, hexahydropyridazinyl, morpholinyl, 1,2-oxazinanyl, 1,3-oxazinanyl, pyrrolidinyl, imidazolidinyl, pyrazolidinyl, diazepanyl, oxazepanyl, azaspirononanyl, diazaspirononanyl, azaspirodecanyl, diazaspirodecanyl, azaspiroundecanyl, and diazaspiroundecanyl, wherein each of the piperazinyl, piperidinyl, azepanyl, hexahydropyrimidinyl, hexahydropyridazinyl, morpholinyl, 1,2-oxazinanyl, 1,3-oxazinanyl, pyrrolidinyl, imidazolidinyl, pyrazolidinyl, diazepanyl, oxazepanyl, azaspirononyl, diazaspirononyl, azaspirodecyl, diazaspirodecyl, azaspiroundecyl, and diazaspiroundecyl groups is optionally substituted with one or two independently selected R³⁰, wherein the one or two independently selected R³⁰ optionally substituting R^{1a} are independently selected from a 1^{st} level substituent, a 2^{nd} level substituent, and a 3^{rd} level substituent as specified herein; more preferred the one or more independently selected R³⁰ optionally substituting R^{1a} are independently selected from the group consisting of methyl, ethyl, —OH, ═O, —OCH₃, —SCH₃, cyclopropyl, 2-hydroxyethyl, 2-(N,N-dimethylamino)ethyl, 2-(N,N-dimethylamino)ethoxy, 2-(methoxy)ethoxy, 2-aminoethyl, 2-(N-methylamino)ethyl, 2-(methoxy)ethyl, 4-methylpiperazinyl, —C(═O)(C₁₋₃ alkyl), —NHC(═O)(C₁₋₃ alkyl), —N(C₁₋₃ alkyl)C(═O)(C₁₋₃ alkyl), —NHS(O)₂(C₁₋₃ alkyl), —N(C₁₋₃ alkyl)S(O)₂(C₁₋₃ alkyl), —(CH₂)₁₋₃COOH, and —NH₂-z(CH₃)_{z}, wherein z is 0, 1, or 2; and each of the C₁₋₃ alkyl groups is optionally substituted with one or two moieties independently selected from the group consisting of —OH, —OCH₃, —SCH₃, cyclopropyl, piperazinyl, 4-methyl-piperazinyl, 4-(2-hydroxyethyl)piperazinyl, 2-(N,N-dimethylamino)ethoxy, and —NH_{2-z}(CH₃)_{z}, wherein z is 0, 1, or 2.

In one embodiment of the kinase inhibitor of formula (III), R¹, R², R³, R^{4a}, R^{4b}, R⁵ and E are independently as defined above (in particular with respect to formula (I), (Ia) or (II)) or below, and R^{1a} is selected from the group consisting of —NH(C₁₋₃ alkyl), piperazinyl, piperidinyl, morpholinyl, pyrrolidinyl, diazepanyl, oxazepanyl, and diazaspirononyl, wherein each of the piperazinyl, piperidinyl, azepanyl, morpholinyl, pyrrolidinyl, diazepanyl, oxazepanyl, and diazaspirononyl groups is optionally substituted with one or two independently selected R³⁰, wherein the one or two independently selected R³⁰ optionally substituting R^{1a} are independently selected from the group consisting of methyl, —OH, ═O, —OCH₃, 2-hydroxyethyl, 2-(N,N-dimethylamino)ethyl, 2-(methoxy)ethoxy, —C(═O)(C₁₋₃ alkyl), —NHC(═O)(C₁₋₃ alkyl), —NHS(O)₂(C₁₋₃ alkyl), and —NH_{2-z}(CH₃)_{z}, wherein z is 0, 1, or 2.

In one embodiment of the kinase inhibitor of formula (III), R¹, R², R³, R^{4a}, R^{4b}, R⁵ and E are independently as defined above (in particular with respect to formula (I), (Ia) or (II)) or below, and R^{1a} is selected from the group consisting of 4-(2-hydroxyethyl)piperazinyl, 4-methylpiperazinyl, 3,4-dimethylpiperazinyl, 4-methyl-1,4-diazepan-1-yl, 3-oxopiperazin-1-yl, 2-methylmorpholin-4-yl, 3-methylpiperazin-1-yl, 3-(2-hydroxyethyl)piperazin-1-yl, 3-(2-hydroxyethyl)-4-methylpiperazin-1-yl, 3-(dimethylamino)piperidin-1-yl, 3-(methoxy)piperidin-1-yl, 3-(hydroxy)piperidin-1-yl, 3-(dimethylamino)pyrrolidin-1-yl, 3-(hydroxy)pyrrolidin-1-yl, 3-(2-methoxyethoxy)pyrrolidin-1-yl, 3-(acetylamino)pyrrolidin-1-yl, 3-(methylsulfonylamino)pyrrolidin-1-yl, 7-methyl-2,7-diazaspiro[4.4]non-2-yl, 4-[2-(dimethylamino)ethyl]-1,4-diazepan-1-yl, 4-(acetyl)-1,4-diazepan-1-yl, 5-oxo-1,4-diazepan-1-yl, and 1,4-oxazepan-4-yl.

In one embodiment of the kinase inhibitor of formula (III), R¹, R², R³, R^{4a}, R^{4b}, R⁵ and E are independently as defined above (in particular with respect to formula (I), (Ia) or (II)) or below, and R^{1a} is selected from the group consisting of C₁₋₃ alkyl, —O(C₁₋₃ alkyl), —S(C₁₋₃ alkyl), —NH(C₁₋₃ alkyl), piperazinyl, morpholinyl, piperidinyl, and pyrrolidinyl, wherein each of the piperazinyl, morpholinyl, piperidinyl, and pyrrolidinyl groups is optionally substituted with one or two moieties independently selected from the group consisting of methyl, ethyl, —OH, —OCH₃, —SCH₃, cyclopropyl, 2-hydroxyethyl, 2-(N,N-dimethylamino)ethyl, 2-(N,N-dimethylamino)ethoxy, 2-aminoethyl, 2-(N-methylamino)ethyl, 2-(methoxy)ethyl, 4-methylpiperazinyl, —C(═O)(C₁₋₃ alkyl), —(CH₂)₁₋₃COOH, and —NH_{2-z}(CH₃)_{z}, wherein z is 0, 1, or 2; and each of the C₁₋₃ alkyl groups is optionally substituted with one or two moieties independently selected from the group consisting of —OH, —OCH₃, —SCH₃, cyclopropyl, piperazinyl, 4-methyl-piperazinyl, 4-(2-hydroxyethyl)piperazinyl, 2-(N,N-dimethylamino)ethoxy, and —NH_{2-z}(CH₃)_{z}, wherein z is 0, 1, or 2.

In one embodiment of the kinase inhibitor of formula (III), R¹, R², R³, R^{4a}, R^{4b}, R⁵ and E are independently as defined above (in particular with respect to formula (I), (Ia) or (II)) or below, and R^{1a} is selected from the group consisting of —NH(C₁₋₃ alkyl), piperazinyl, piperidinyl, and pyrrolidinyl, wherein the piperazinyl group is optionally substituted with one or two moieties independently selected from the group consisting of 2-hydroxyethyl, methyl, —CH₂COOH, and —C(═O)CH₃; the piperidinyl group is optionally substituted with one or two moieties independently selected from the group consisting of —NH₂ and 4-methylpiperazinyl; the pyrrolidinyl is optionally substituted with one or two —OH; and each of the C₁₋₃ alkyl groups is optionally substituted with one or two moieties independently selected from the group consisting of —OH, —OCH₃, and —NH_{2-z}(CH₃)_{z}, wherein z is 0, 1, or 2.

In one embodiment of the kinase inhibitor of formula (III), R¹, R², R³, R^{4a}, R^{4b}, R⁵ and E are independently as defined above (in particular with respect to formula (I), (Ia) or (II)) or below, and R^{1a} is selected from the group consisting of 4-(2-hydroxyethyl)piperazinyl, 4-methylpiperazinyl, 4-acetylpiperazinyl, (2-hydroxyethyl)amino, 4-aminopiperidinyl, 4-(4-methylpiperazinyl)piperidinyl, (4-carboxymethylpiperazinyl), and 3-hydroxypyrrolidinyl, such as from the group consisting of 4-(2-hydroxyethyl)piperazinyl, 4-methylpiperazinyl, 4-acetylpiperazinyl, and (2-hydroxyethyl)amino.

In one embodiment of the kinase inhibitor of formula (III), R¹, R², R³, R^{4a}, R^{4b}, R⁵ and E are independently as defined above (in particular with respect to formula (I), (Ia) or (II)) or below, and R^{1a} is a leaving group (e.g., a halogen (such as Cl, Br, or F), nitro, benzotriazol-1-yloxy, C₁-C₁₀ alkylsulfonate, C₁-C₁₀ haloalkylsulfonate, nonaflate (CF₃CF₂CF₂CF₂SO³—), CF₃C(═O)O—, phenylsulfonate (wherein the phenyl is optionally substituted with 1, 2, or 3 groups which are each independently selected from halogen and C₁-C₄ alkyl), or an ammonium salt of the formula —[N(R^{x})(R^{y})(R^{z})]⁺[G]⁻, wherein R^{x}, R^{y}, and R^{z} are independently hydrogen or alkyl, and G is the conjugate base of a strong acid (e.g., G⁻ is Cl⁻)).

In one embodiment of the kinase inhibitor of formula (III), R¹, R², R³, R^{4a}, R^{4b}, R⁵ and E are independently as defined above (in particular with respect to formula (I), (Ia) or (II)) or below, and R^{1a} is selected from the group consisting of:

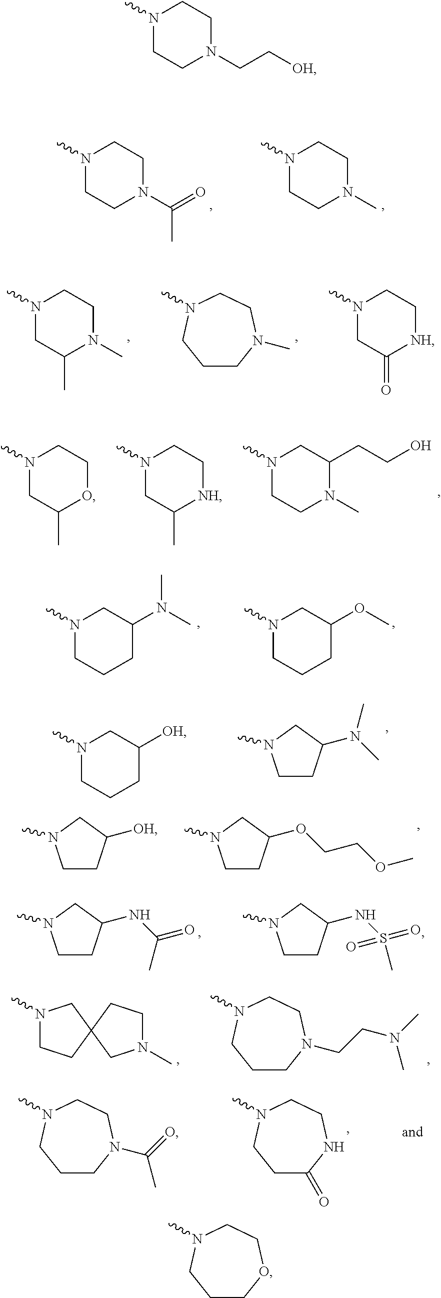

wherein [symbol] represent the bond by which R^{1a} is bound to the remainder of the compound. selected from the group consisting of:

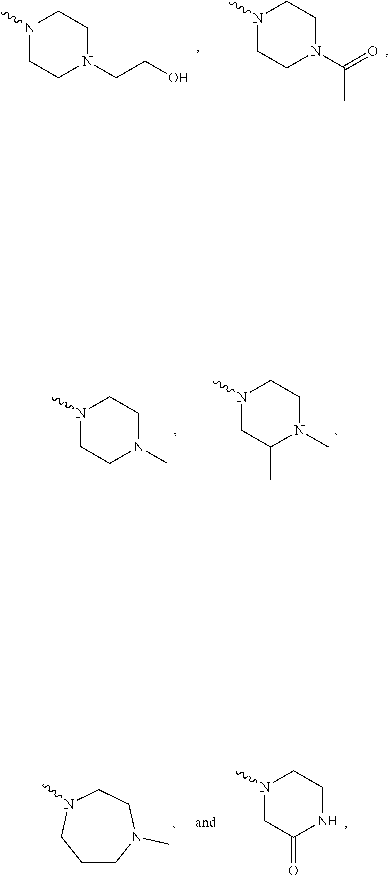

wherein [symbol] represents the bond by which R^{1a} is bound to the remainder of the compound.

In one, embodiment of the kinase inhibitor of formula (III), R¹, R², R³, R^{4a}, R^{4b}, R⁵ and E are independently as defined above (in particular with respect to formula (I), (Ia) or (II)) or below, and R^{1a} is selected from the group consisting of C₁₋₃ alkyl, —O(C₁₋₃ alkyl), —S(C₁₋₃ alkyl), —NH(C₁₋₃ alkyl), piperazinyl, morpholinyl, piperidinyl, pyrrolidinyl, and azepanyl wherein each of the piperazinyl, morpholinyl, piperidinyl, pyrrolidinyl, and azepanyl groups is optionally substituted with one or two moieties independently selected from the group consisting of methyl, ethyl, —OH, —OCH₃, —SCH₃, cyclopropyl, 2-hydroxyethyl, 2-(N,N-dimethylamino)ethyl, 2-(N,N-dimethylamino)ethoxy, 2-aminoethyl, 2-(N-methylamino)ethyl, 2-(methoxy)ethyl, 4-methylpiperaanyl, —C(═O)(C₁₋₃ alkyl), —(CH₂)₁₋₃COOH, and —NH_{2-z}(CH₃)_{z}, wherein z is 0, 1, or 2; and each of the C₁₋₃ alkyl groups is optionally substituted with one or two moieties independently selected from the group consisting of —OH, —OCH₃, —SCH₃, cyclopropyl, piperazinyl, 4-methyl-piperazinyl, 4-(2-hydroxyethyl)piperazinyl, 2-(N,N-dimethylamino)ethoxy, and —NH_{2-z}(CH₃)_{z}, wherein z is 0, 1, or 2.

In one embodiment of the kinase inhibitor of formula (III), R¹, R², R³, R^{4a}, R^{4b}, R⁵ and E are independently as defined above (in particular with respect to formula (I), (Ia) or (II)) or below, and R^{1a} is selected from the group consisting of —NH(C₁₋₃ alkyl), piperazinyl, piperidinyl, pyrrolidinyl, and azepanyl wherein the piperazinyl group is optionally substituted with one or two moieties independently selected from the group consisting of 2-hydroxyethyl, methyl, —CH₂COOH, and —C(═O)CH₃; the piperidinyl group is optionally substituted with one to three R³⁰ independently selected from the group consisting of methyl, —NH₂ and 4-methylpiperazinyl; the pyrrolidinyl is optionally substituted with one or two R³⁰ being —OH; and each of the C₁₋₃ alkyl groups is optionally substituted with one or two R³⁰ independently selected from the group consisting of —OH, —OCH₃, and —NH_{2-z}(CH₃)_{z}, wherein z is 0, 1, or 2.

In one embodiment of the kinase inhibitor of formula (III), R¹, R², R³, R^{4a}, R^{4b}, R⁵ and E are independently as defined above (in particular with respect to formula (I), (Ia) or (II)) or below, and R^{1a} is selected from the group consisting of piperidinyl substituted with one to three moieties independently selected from the group consisting of C₁ to C₄ alkyl; piperazinyl group substituted with one or two R³⁰ independently selected from the group consisting of 2-hydroxyethyl and C₁ to C₄ alkyl; azepanyl substituted with one to three R³⁰ independently selected from the group consisting of C₁ to C₄ alkyl; morpholinyl; and C₁₋₃ alkyl group substituted with R³⁰ being —NH_{2-z}(CH₃)_{z}, wherein z is 0, 1, or 2.

In one embodiment of the kinase inhibitor of formula (III), R¹, R², R³, R^{4a}, R^{4b}, R⁵ and E are independently as defined above (in particular with respect to formula (I), (Ia) or (II)) or below, and R^{1a} is selected from the group consisting of piperidinyl substituted with one to three moieties independently selected from the group consisting of C₁ to C₄ alkyl; piperazinyl group substituted with one or two R³⁰ independently selected from the group consisting of 2-hydroxyethyl and C₁ to C₄ alkyl; azepanyl substituted with one to three R³⁰ independently selected from the group consisting of C₁ to C₄ alkyl; morpholinyl; and C₁₋₃ alkyl group substituted with R³⁰ being —NH_{2-z}(CH₃)_{z}, wherein z is 0, 1, or 2.

In one embodiment of the kinase inhibitor of formula (III), R¹, R², R³, R^{4a}, R^{4b}, R⁵ and E are independently as defined above (in particular with respect to formula (I), (Ia) or (II)) or below, and R^{1a} is piperidinyl substituted with one to three R³⁰ independently selected from the group consisting of C₁ to C₄ alkyl.

In one embodiment of the kinase inhibitor of formula (III), R¹, R², R³, R^{4a}, R^{4b}, R⁵ and E are independently as defined above (in particular with respect to formula (I), (Ia) or (II)) or below, and R^{1a} is piperidinyl substituted with one to three R³⁰ methyl.

In one embodiment of the kinase inhibitor of formula (III), R¹, R², R³, R^{4a}, R^{4b}, R⁵ and E are independently as defined above (in particular with respect to formula (I), (Ia) or (II)) or below, and R^{1a} is selected from the group consisting of 1-methylpiperidinyl, 1,2-dimethylpiperidinyl, 1,2,6-trimethylpiperidinyl, 1-methylazepanyl, 4-(2-hydroxyethyl)piperazinyl, 1-methyl-4-(2-hydroxyethyl)piperazinyl, 4-methylpiperazinyl, 4-acetylpiperazinyl, and (2-hydroxyethyl)amino.

In one embodiment of the kinase inhibitor of formula (III), R¹, R², R³, R^{4a}, R^{4b}, R⁵ and E are independently as defined above (in particular with respect to formula (I), (Ia) or (II)) or below, and R^{1a} is selected from the group consisting of:

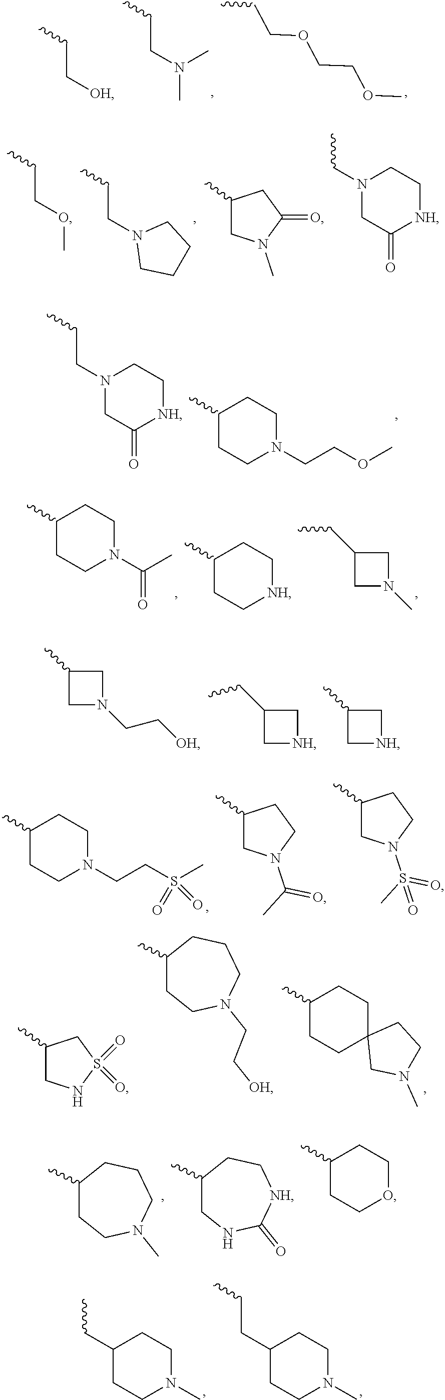

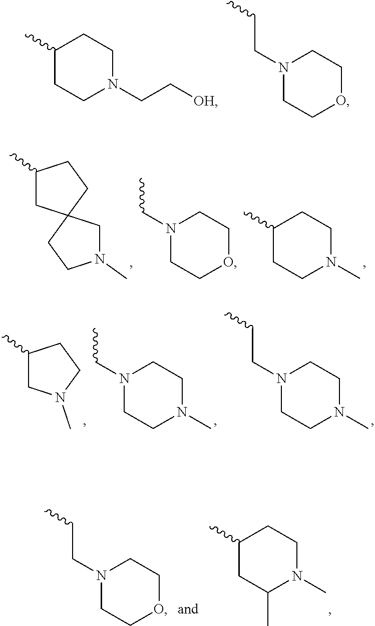

wherein [symbol] represents the bond by which R^{1a} is bound to the remainder of the compound. Preferably, R^{1a} is selected from the group consisting of:

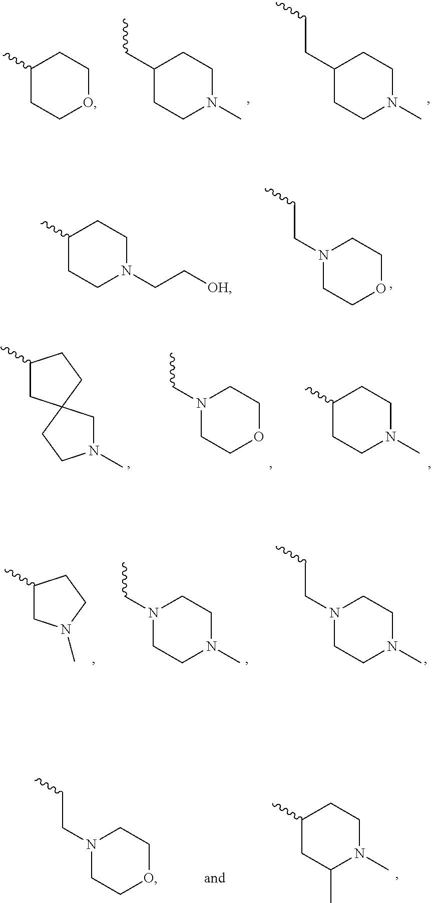

wherein [symbol] represents the bond by which R^{1a} is bound to the remainder of the compound. More preferably, R^{1a} is selected from the group consisting of:

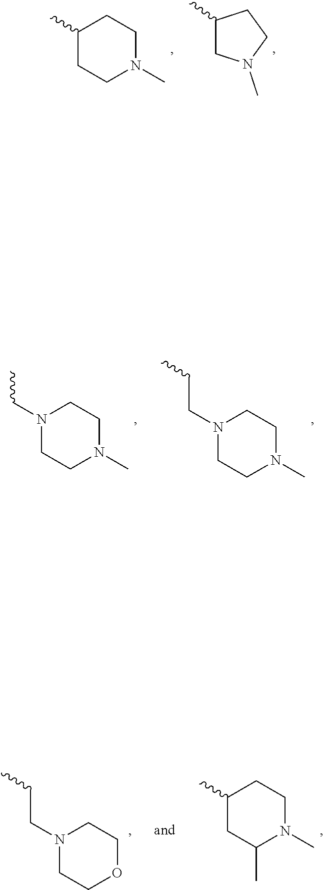

wherein [symbol] represents the bond by which R^{1a} is bound to the remainder of the compound

In one embodiment the kinase inhibitor has the formula (IV):

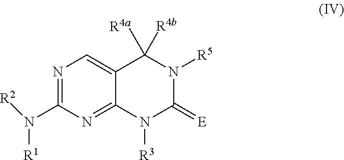

wherein R¹, R², R³, R^{4a}, R^{4b}, R⁵ and E are independently as defined above (in particular with respect to formula (I), (Ia), (II) or (III)) or below, and Q of R¹ is selected from the group consisting of a 3- to 10-membered mono or bicyclic cycloalkyl, aryl, heterocyclyl, heteroaryl, wherein each of the cycloalkyl, aryl, heterocyclyl, and heteroaryl groups is optionally substituted with one or more independently selected R³⁰.

In one embodiment of the kinase inhibitor of formula (IV), R¹, R², R³, R^{4a}, R^{4b}, R⁵ and E are independently as defined above (in particular with respect to formula (I), (Ia), (II) or (III)) or below, and Q is selected from the group consisting of a bond, 3- to 10-membered mono or bicyclic cycloalkyl, aryl, and heteroaryl, wherein each of the cycloalky, aryl and heteroaryl groups is optionally substituted with one or more independently selected R³⁰.

In one embodiment of the kinase inhibitor of formula (IV), R¹, R², R³, R^{4a}, R^{4b}, R⁵ and E are independently as defined above (in particular with respect to formula (I), (Ia), (II) or (III)) or below, and Q is selected from the group consisting of a bond, C₆₋₁₀ aryl, and 5- or 6-membered heteroaryl, wherein each of the C₆₋₁₀ aryl, and 5- or 6-membered heteroaryl is optionally substituted with one or more R³⁰ independently selected from the group consisting of C₁₋₈ alkyl, halogen, —OR¹¹, —N(R¹²)(R¹³), —SR¹¹, C(═O)R¹¹, wherein each of the C₁₋₈ alkyl, halogen, —OR¹¹, —N(R¹²)(R¹³), —SR¹¹, C(═O)R¹¹ is optionally substituted with one or more independently selected R³⁰.

In one embodiment of the kinase inhibitor of formula (IV), R¹, R², R³, R^{4a}, R^{4b}, R⁵ and E are independently as defined above (in particular with respect to formula (I), (Ia), (II) or (III)) or below, and Q is C₆₋₁₀ aryl, wherein the C₆₋₁₀ aryl is optionally substituted with one or more R³⁰ being —OR¹¹, wherein R¹¹ is independently selected from C₁₋₁₂ alkyl.

In one embodiment of the kinase inhibitor of formula (IV), R¹, R², R³, R^{4a}, R^{4b}, R⁵ and E are independently as defined above (in particular with respect to formula (I), (Ia), (II) or (III)) or below, and Q is C₆₋₁₀ aryl, wherein the C₆₋₁₀ aryl is optionally substituted with one or more R³⁰ being —OR¹¹, wherein R¹¹ is independently selected from C₁₋₈ alkyl.

In one embodiment of the kinase inhibitor of formula (IV), R¹, R², R³, R^{4a}, R^{4b}, R⁵ and E are independently as defined above (in particular with respect to formula (I), (Ia), (II) or (III)) or below, and Q is C₆₋₁₀ aryl, wherein the C₆₋₁₀ aryl is optionally substituted with one or more R³⁰ being —OR¹¹, wherein R¹¹ is independently selected from C₁₋₄ alkyl.

In one embodiment of the kinase inhibitor of formula (IV), R¹, R², R³, R^{4a}, R^{4b}, R⁵ and E are independently as defined above (in particular with respect to formula (I), (Ia), (II) or (III)) or below, and Q is C₆ aryl, wherein the C₆ aryl is optionally substituted with one or more R³⁰ being —OR¹¹, wherein R¹¹ is independently selected from C₁₋₄ alkyl.

In one embodiment of the kinase inhibitor of formula (IV), R¹, R², R³, R^{4a}, R^{4b}, R⁵ and E are independently as defined above (in particular with respect to formula (I), (Ia), (II) or (III)) or below, and Q is C₆ aryl, wherein the C₆ aryl is optionally substituted with one or more R³⁰ being —OR¹¹, wherein R¹¹ is methyl.

In one embodiment of the kinase inhibitor of formulas (IV), R¹, R², R³, R^{4a}, R^{4b}, R⁵ and E are independently as defined above (in particular with respect to formula (I), (Ia), (II) or (III)) or below, and R^{1a} and —OR¹¹ are in meta-position with respect to each other.

In one embodiment of the kinase inhibitor of formulas (IV), R¹, R², R³, R^{4a}, R^{4b}, R⁵ and E are independently as defined above (in particular with respect to formula (I), (Ia), (II) or (III)) or below, and R^{1a} and the bond between R¹ (such as between Q of R¹) and N in Formula (I), (Ia), (II) or (III) are in meta-position or in para-position with respect to each other.

In one embodiment of the kinase inhibitor of formulas (IV), R¹, R², R³, R^{4a}, R^{4b}, R⁵ and E are independently as defined above (in particular with respect to formula (I), (Ia), (II) or (III)) or below, and R^{1a} and the bond between R¹ (such as between Q of R¹) and N in Formula (I), (Ia), (II) or (III) are in para-position with respect to each other.

In one embodiment of the kinase inhibitor of formulas (IV), R¹, R², R³, R^{4a}, R^{4b}, R⁵ and E are independently as defined above (in particular with respect to formula (I), (Ia), (II) or (III)) or below, and —OR¹¹ and the bond between R¹ (such as between Q of R¹) and N in Formula (I), (Ia), (II) or (III) are in ortho-position with respect to each other.

In one embodiment of the kinase inhibitor of formulas (IV), R¹, R², R³, R^{4a}, R^{4b}, R⁵ and E are independently as defined above (in particular with respect to formula (I), (Ia), (II) or (III)) or below, and Q is C₆₋₁₀ aryl; R¹ and R³ join together via a group L′ to form a moiety Q-L′-R³; and the bond between Q and L′ and R^{1a} are in ortho-position with respect to each other.

In particular of such embodiments: (i) Q is C₆ aryl, wherein the C₆ aryl is optionally substituted with one or more R³⁰ being —OR¹¹, wherein R¹¹ is independently selected from C₁₋₄ alkyl; and (ii) R^{1a} is selected from the group consisting of:

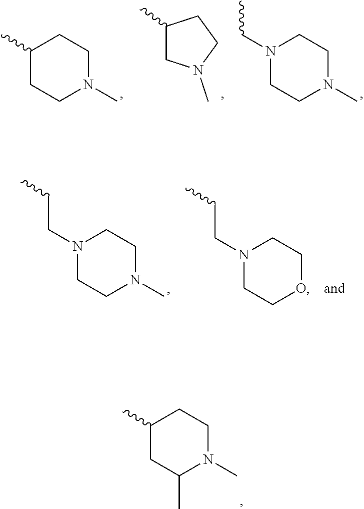

wherein [symbol] represents the bond by which R^{1a} is bound to the remainder of the compound.

In one embodiment, the kinase inhibitor has the formula (V):

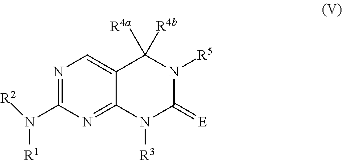

wherein R¹, R², R³, R^{4a}, R^{4b} and R⁵ are independently as defined above (in particular with respect to formula (I), (Ia), (II), (III) or (IV)) or below, and E is O.

In an alternative embodiment, the kinase inhibitor has the formula (Va):

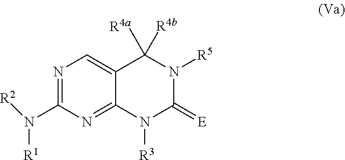

wherein R¹, R², R³, R^{4a}, R^{4b} and R⁵ are independently as defined above (in particular with respect to formula (I), (Ia), (II), (III) or (IV)) or below, and E is S.

In one embodiment, the kinase inhibitor has the formula (VI):

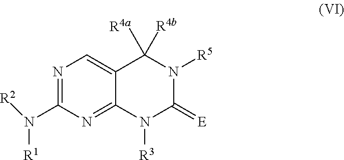

wherein R¹, R², R^{4a}, R^{4b}, R⁵ and E are independently as defined above (in particular with respect to formula (I), (Ia), (II), (III), (IV), (V) or (Va)) or below, and R³ H, alkyl, alkenyl, alkynyl, cycloalkyl, aryl, heterocyclyl, heteroaryl, wherein each of the alkyl, alkenyl, alkynyl, cycloalkyl, aryl, heterocyclyl, and heteroaryl groups is optionally substituted with one or more independently selected R³⁰,

In one embodiment of the kinase inhibitor of formulas (VI), R¹, R², R³, R^{4a}, R^{4b}, R⁵ and E are independently as defined above (in particular with respect to formula (I), (Ia), (II), (III), (IV), (V) or (Va)) or below, and R³⁰ optionally substituting R³ are selected from (i) C₁₋₃ alkyl, phenyl, thiazolidinyl, halogen, —NH₂, —NHS(O)₂(C₁₋₃ alkyl), —NHC(═O) (C₁₋₃ alkyl), and —NHC(═NH)NH_{z-2}(C₁₋₃ alkyl)_{z}, wherein z is 0, 1, or 2 and each C₁₋₃ alkyl is independently methyl, ethyl, propyl or isopropyl; (ii) methyl, ethyl, propyl, isopropyl, phenyl, ═O, and ═S; or (iii) methyl, ethyl, propyl, isopropyl, halogen, and —CF₃.

In one embodiment of the kinase inhibitor of formulas (VI), R¹, R², R^{4a}, R^{4b}, R⁵ and E are independently as defined above (in particular with respect to formula (I), (Ia), (II), (III), (IV), (V) or (Va)) or below, and R³ is selected from the group consisting of alkyl, cycloalkyl, aryl, and heteroaryl, wherein each of the alkyl, cycloalkyl, aryl, and heteroaryl groups is optionally substituted with one or more independently selected R³⁰,

In one embodiment of the kinase inhibitor of formulas (VI), R¹, R², R^{4a}, R^{4b}, R⁵ and E are independently as defined above (in particular with respect to formula (I), (Ia), (II), (III), (IV), (V) or (Va)) or below, and R³ is selected from the groups consisting of methyl (Me), ethyl (Et), propyl, iso-propyl (also called 2-propyl or 1-methylethyl), butyl, iso-butyl, tert-butyl, n-pentyl, iso-pentyl, sec-pentyl, neo-pentyl, 1,2-dimethyl-propyl, iso-amyl, n-hexyl, iso-hexyl, sec-hexyl, n-heptyl, iso-heptyl, n-octyl, 2-ethyl-hexyl, n-nonyl, n-decyl, n-undecyl, n-dodecyl, cyclopropyl, cyclopropenyl, cyclobutyl, cyclobutenyl, cyclopentyl, cyclopentenyl, cyclopentynyl, cyclohexyl, cyclohexenyl, cyclohexynyl, cycloheptyl, cycloheptenyl, cycloheptynyl, cyclooctyl, cyclooctenyl, cyclooctynyl, cyclononyl, cyclononenyl, cyclononynyl, cylcodecyl, cylcodecenyl, cylcodecynyl, adamantly, spiro[3,3]heptyl, spiro[3,4]octyl, spiro[4,3]octyl, bicyclo[4.1.0]heptyl, bicyclo[3.2.0]heptyl, bicyclo[2.2.1]heptyl, bicyclo[2.2.2]octyl, bicyclo[5.1.0]octyl, and bicyclo[4.2.0]octyl, cyclopropenylium, cyclopentadienyl, phenyl, indenyl, naphthyl, azulenyl, fluorenyl, anthryl, phenanthryl, furanyl, thienyl, oxazolyl, isoxazolyl, oxadiazolyl (1,2,5- and 1,2,3-), pyrrolyl, imidazolyl, pyrazolyl, triazolyl (1,2,3- and 1,2,4-), tetrazolyl, thiazolyl, isothiazolyl, thiadiazolyl (1,2,3- and 1,2,5-), pyridyl (also called pyridinyl), pyrimidinyl, pyrazinyl, triazinyl (1,2,3-, 1,2,4-, and 1,3,5-), benzofuranyl (1- and 2), indolyl, isoindolyl, benzothienyl (1- and 2-), 1H-indazolyl, benzimidazolyl, benzoxazolyl, indoxazinyl, benzisoxazolyl, benzothiazolyl, benzisothiazolyl, benzotriazolyl, quinolinyl, isoquinolinyl, benzodiazinyl, quinoxalinyl, quinazolinyl, benzotriazinyl (1,2,3- and 1,2,4-benzotriazinyl), pyridazinyl, phenoxazinyl, thiazolopyridinyl, pyrrolothiazolyl, phenothiazinyl, isobenzofuranyl, chromenyl, xanthenyl, phenoxathiinyl, pyrrolizinyl, indolizinyl, indazolyl, purinyl, quinolizinyl, phthalazinyl, naphthyridinyl (1,5-, 1,6-, 1,7-, 1,8-, and 2,6-), cinnolinyl, pteridinyl, carbazolyl, phenanthridinyl, acridinyl, perimidinyl, phenanthrolinyl (1,7-, 1,8-, 1,10-, 3,8-, and 4,7-), phenazinyl, oxazolopyridinyl, isoxazolopyridinyl, pyrrolooxazolyl, and pyrrolopyrrolyl. Exemplary 5- or 6-membered heteroaryl groups include furanyl, thienyl, oxazolyl, isoxazolyl, oxadiazolyl (1,2,5- and 1,2,3-), pyrrolyl, imidazolyl, pyrazolyl, triazolyl (1,2,3- and 1,2,4-), thiazolyl, isothiazolyl, thiadiazolyl (1,2,3- and 1,2,5), pyridyl, pyrimidinyl, pyrazinyl, triazinyl (1,2,3-, 1,2,4-, and 1,3,5-), and pyridazinyl which are optionally substituted with one or more independently selected R³⁰. Preferably, the substituent other than hydrogen is a 1^{st} level substituent, a 2^{nd} level substituent, or a 3^{rd} level substituent as specified herein, such as halogen, CN, nitro, —OR¹¹ (e.g., —OH), —SR¹¹ (e.g., —SH), —N(R¹²)(R¹³) (e.g., —NH₂), alkyl (e.g., C₁₋₆ alkyl), alkenyl (e.g., C₂₋₆ alkenyl), and alkynyl (e.g., C₂₋₆ alkynyl). Examples of a substituted heteroaryl include 2,4-dimethylpyridin-3-yl, 2-methyl-4-bromopyridin-3-yl, 3-methyl-2-pyridin-2-yl, 3-chloro-5-methylpyridin-4-yl, 4-chloro-2-methylpyridin-3-yl, 3,5-dimethylpyridin-4-yl, 2-methylpyridin-3-yl, 2-chloro-4-methyl-thien-3-yl, 1,3,5-trimethylpyrazol-4-yl, 3,5-dimethyl-1,2-dioxazol-4-yl, 1,2,4-trimethylpyrrol-3-yl, 3-phenylpyrrolyl, 2,3′-bifuryl, 4-methylpyridyl, 2-, or 3-ethylindolyl.

In one embodiment of the kinase inhibitor of formulas (VI), R¹, R², R^{4a}, R^{4b}, R⁵ and E are independently as defined above (in particular with respect to formula (I), (Ia), (II), (III), (IV), (V) or (Va)) or below, and R³ is selected from the group consisting of C₁₋₈ alkyl, C₃₋₁₀ cycloalkyl, C₆₋₁₀ aryl, and 5- to 8-membered heteroaryl, wherein each of the C₁₋₈ alkyl, C₃₋₁₀ cycloalkyl, C₆₋₁₀ aryl, and 5- to 8-membered heteroaryl groups is optionally substituted with one or more independently selected R³⁰

In one embodiment of the kinase inhibitor of formulas (VI), R¹, R², R^{4a}, R^{4b}, R⁵ and E are independently as defined above (in particular with respect to formula (I), (Ia), (II), (III), (IV), (V) or (Va)) or below, and R³ is selected from the groups consisting of methyl (Me), ethyl (Et), propyl, iso-propyl (also called 2-propyl or 1-methylethyl), butyl, iso-butyl, tert-butyl, n-pentyl, iso-pentyl, sec-pentyl, neo-pentyl, 1,2-dimethyl-propyl, iso-amyl, n-hexyl, iso-hexyl, sec-hexyl, n-heptyl, iso-heptyl, n-octyl, 2-ethyl-hexyl, cyclopropyl, cyclopropenyl, cyclobutyl, cyclobutenyl, cyclopentyl, cyclopentenyl, cyclopentynyl, cyclohexyl, cyclohexenyl, cyclohexynyl, cycloheptyl, cycloheptenyl, cycloheptynyl, cyclooctyl, cyclooctenyl, cyclooctynyl, cyclononyl, cyclononenyl, cyclononynyl, cylcodecyl, cylcodecenyl, cylcodecynyl, adamantly, spiro[3,3]heptyl, spiro[3,4]octyl, spiro[4,3]octyl, bicyclo[4.1.0]heptyl, bicyclo[3.2.0]heptyl, bicyclo[2.2.1]heptyl, bicyclo[2.2.2]octyl, bicyclo[5.1.0]octyl, and bicyclo[4.2.0]octyl, phenyl, naphthyl, pyridyl (also called pyridinyl), pyrimidinyl, pyrazinyl, triazinyl (1,2,3-, 1,2,4-, and 1,3,5-), benzofuranyl (1- and 2), indolyl, isoindolyl, benzothienyl (1- and 2-), 1H-indazolyl, benzimidazolyl, benzoxazolyl, indoxazinyl, benzisoxazolyl, benzothiazolyl, benzisothiazolyl, benzotriazolyl, quinolinyl, isoquinolinyl, benzodiazinyl, quinoxalinyl, quinazolinyl, benzotriazinyl (1,2,3- and 1,2,4-benzotriazinyl), pyridazinyl, phenoxazinyl, thiazolopyridinyl, pyrrolothiazolyl, phenothiazinyl, isobenzofuranyl, chromenyl, xanthenyl, phenoxathiinyl, pyrrolizinyl, indolizinyl, indazolyl, purinyl, quinolizinyl, phthalazinyl, naphthyridinyl (1,5-, 1,6-, 1,7-, 1,8-, and 2,6-), cinnolinyl, pteridinyl, carbazolyl, phenanthridinyl, acridinyl, perimidinyl, phenanthrolinyl (1,7-, 1,8-, 1,10-, 3,8-, and 4,7-), phenazinyl, oxazolopyridinyl, isoxazolopyridinyl, pyrrolooxazolyl, and pyrrolopyrrolyl. Exemplary 5- or 6-membered heteroaryl groups include furanyl, thienyl, oxazolyl, isoxazolyl, oxadiazolyl (1,2,5- and 1,2,3-), pyrrolyl, imidazolyl, pyrazolyl, triazolyl (1,2,3- and 1,2,4-), thiazolyl, isothiazolyl, thiadiazolyl (1,2,3- and 1,2,5), pyridyl, pyrimidinyl, pyrazinyl, triazinyl (1,2,3-, 1,2,4-, and 1,3,5-), and pyridazinyl which are optionally substituted with one or more independently selected R³⁰. Preferably, the substituent other than hydrogen is a 1^{st} level substituent, a 2^{nd} level substituent, or a 3^{rd} level substituent as specified herein, such as halogen, CN, nitro, —OR¹¹ (e.g., —OH), —SR¹¹ (e.g., —SH), —N(R¹²)(R¹³) (e.g., —NH₂), alkyl (e.g., C₁₋₆ alkyl), alkenyl (e.g., C₂₋₆ alkenyl), and alkynyl (e.g., C₂₋₆ alkynyl). Examples of a substituted heteroaryl include 2,4-dimethylpyridin-3-yl, 2-methyl-4-bromopyridin-3-yl, 3-methyl-2-pyridin-2-yl, 3-chloro-5-methylpyridin-4-yl, 4-chloro-2-methylpyridin-3-yl, 3,5-dimethylpyridin-4-yl, 2-methylpyridin-3-yl, 2-chloro-4-methyl-thien-3-yl, 1,3,5-trimethylpyrazol-4-yl, 3,5-dimethyl-1,2-dioxazol-4-yl, 1,2,4-trimethylpyrrol-3-yl, 3-phenylpyrrolyl, 2,3′-bifuryl, 4-methylpyridyl, 2-, or 3-ethylindolyl.

In one embodiment of the kinase inhibitor of formulas (VI), R¹, R², R^{4a}, R^{4b}, R⁵ and E are independently as defined above (in particular with respect to formula (I), (Ia), (II), (III), (IV), (V) or (Va)) or below, and R³ is selected from the group consisting of C₁₋₈ alkyl, C₃₋₁₀ cycloalkyl, C₆₋₁₀ aryl, and 5- to 8-membered heteroaryl, wherein each of the C₁₋₈ alkyl, C₃₋₁₀ cycloalkyl, C₆₋₁₀ aryl, and 5- to 8-membered heteroaryl groups is optionally substituted with one or more R³⁰ independently selected from the group consisting of C₁₋₈ alkyl, halogen, —OR¹¹, —N(R¹²)(R¹³), —SR¹¹, C(═O)R¹¹, wherein each of C₁₋₈ alkyl, halogen, —OR¹¹, —N(R¹²)(R¹³), —SR¹¹, and C(═O)R¹¹ is optionally substituted with one or more independently selected R³⁰,

In one embodiment of the kinase inhibitor of formulas (VI), R¹, R², R^{4a}, R^{4b}, R⁵ and E are independently as defined above (in particular with respect to formula (I), (Ia), (II), (III), (IV), (V) or (Va)) or below, and R³ is selected from the group consisting of C₁₋₈ alkyl, C₃₋₁₀ cycloalkyl, C₆₋₁₀ aryl, and 5- to 8-membered heteroaryl, wherein each of the C₁₋₈ alkyl, C₃₋₁₀ cycloalkyl, C₆₋₁₀ aryl, and 5- to 8-membered heteroaryl groups is optionally substituted with one or more R³⁰ independently selected from the group consisting of C₁₋₈ alkyl, halogen, —OR¹¹, —N(R¹²)(R¹³), —SR¹¹, and C(═O)R¹¹.

In one embodiment of the kinase inhibitor of formulas (VI), R¹, R², R^{4a}, R^{4b}, R⁵ and E are independently as defined above (in particular with respect to formula (I)) or below, and R³ is selected from the group consisting of C₁₋₆ alkyl, C₃₋₆ cycloalkyl, C₆ aryl, and 5- or 6-membered heteroaryl, wherein each of the C₁₋₆ alkyl, C₃₋₆ cycloalkyl, C₆ aryl, and 5- or 6-membered heteroaryl groups is optionally substituted with one or more R³⁰ independently selected from the group consisting of C₁₋₆ alkyl, and OR¹¹, wherein R¹¹ C₁-12 alkyl.

In one embodiment of the kinase inhibitor of formulas (VI), R¹, R², R^{4a}, R^{4b}, R⁵ and E are independently as defined above (in particular with respect to formula (I), (Ia), (II), (III), (IV), (V) or (Va)) or below, and R³ is selected from the group consisting of C₁₋₄ alkyl, C₆ aryl, and 6-membered N-containing heteroaryl, wherein each of the C₁₋₄ alkyl, C₆ aryl, and 6-membered heteroaryl groups is optionally substituted with one or more R³⁰ independently selected from the group consisting of C₁₋₄ alkyl, and OR¹¹, wherein R¹¹ C₁₋₄ alkyl.

In one embodiment of the kinase inhibitor of formulas (VI), R¹, R², R^{4a}, R^{4b}, R⁵ and E are independently as defined above (in particular with respect to formula (I), (II), (III), (IV), (V) or (Va)) or below, and R³ is selected from the group consisting of methyl, ethyl, phenyl, and pyridyl, wherein each of the methyl, ethyl, phenyl, and pyridyl is optionally substituted with one or more R³⁰ being methoxy.

In one embodiment of the kinase inhibitor of formulas (VI), R¹, R², R^{4a}, R^{4b}, R⁵ and E are independently as defined above (in particular with respect to formula (I), (Ia), (II), (III), (IV), (V) or (Va)) or below, and R³ is selected from the group consisting of methyl, 2-methoxyethyl, methoxyphenyl, and 3-methoxypyridyl, and 1,3-dimethoxyphenyl.

In one embodiment of the kinase inhibitor of formulas (VI), R¹, R², R^{4a}, R^{4b}, R⁵ and E are independently as defined above (in particular with respect to formula (I), (Ia), (II), (III), (IV), (V) or (Va)) or below, and R³ is methoxypyridyl.

In one embodiment of the kinase inhibitor of formulas (VI), R¹, R², R³, R^{4a}, R^{4b}, R⁵ and E are independently as defined above (in particular with respect to formula (I), (Ia), (II), (III), (IV), (V) or (Va)) or below, and wherein R¹ and R³ join together via a group L′ to form a moiety R¹-L′-R³ and, optionally, wherein R³ is a bond. In certain of such embodiments R¹ and R³ join together via a group L′ to form a moiety Q-L′-R³ and, optionally, wherein R³ is a bond-

In one embodiment, the kinase inhibitor has the formula (VII):

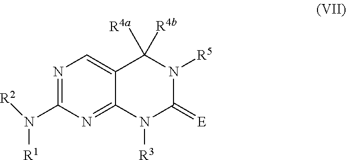

wherein R¹, R², R³, R⁵ E are independently as defined above (in particular with respect to formula (I), (Ia), (II), (III), (IV), (V), (Va) or (VI)) or below, and R^{4a} and R^{4b} are independently selected from the group consisting of H, C₁₋₃ alkyl, and C₂₋₃ alkyloxy; or optionally R^{4a} and R^{4b} may join together to form, together with the carbon to which they are attached, C₃₋₈ cycloalkyl or a 4- to 6-membered heterocyclyl comprising at least one 0 as the only heteroatom element, wherein in case that the 4- to 6-membered heterocyclyl comprises more than one 0, different 0 are not directly bound to each other.

In one embodiment of the kinase inhibitor has the formula (VII), R¹, R², R³, R⁵ E are independently as defined above (in particular with respect to formula (I), (Ia), (II), (III), (IV), (V), (Va) or (VI)) or below, and R^{4a} and R^{4b} are independently selected from the group consisting of H, methyl, ethyl, n-propyl, iso-propyl (also called 2-propyl), methoxymethyl (—CH₂—O—CH₃); or optionally R^{4a} and R^{4b} may join together to form a cyclopropyl, cyclobutyl, cyclopentyl, cyclohexyl, cycloheptyl, cyclooctyl, azepin and N-methyl azepin..

In one embodiment of the kinase inhibitor has the formula (VII), R¹, R², R³, R⁵ E are independently as defined above (in particular with respect to formula (I), (Ia), (II), (III), (IV), (V), (Va) or (VI)) or below, and R^{4a}, R^{4b}, are independently selected from the group consisting of H, C₁ or C₂ alkyl; or optionally R^{4a} and R^{4b} may join together to form, together with the carbon to which they are attached, C₃ or C₄ cycloalkyl or a 4-membered heterocyclyl comprising one O as the only heteroatom element.

In one embodiment of the kinase inhibitor has the formula (VII), R¹, R², R³, R⁵ E are independently as defined above (in particular with respect to formula (I), (Ia), (II), (III), (IV), (V), (Va) or (VI)) or below, and R^{4a} and R^{4b} are independently selected from the group consisting of H, C₁ or C₂ alkyl; or optionally R^{4a} and R^{4b} may join together to form, together with the carbon to which they are attached, C₃ cycloalkyl.

In one embodiment of the kinase inhibitor has the formula (VII), R¹, R², R³, R⁵ E are independently as defined above (in particular with respect to formula (I), (Ia), (II), (III), (IV), (V), (Va) or (VI)) or below, and R^{4a} and R^{4b} are independently selected from the group consisting of H, and methyl; or optionally R^{4a} and R^{4b} may join together to form, together with the carbon to which they are attached, cyclopropy.

In one embodiment of the kinase inhibitor has the formula (VII), R¹, R², R³, R⁵ E are independently as defined above (in particular with respect to formula (I), (Ia), (II), (III), (IV), (V), (Va) or (VI)) or below, and both R^{4a} and R^{4b} are H.

In one alternative embodiment, the kinase inhibitor has the formula (VII), R¹, R², R³, R⁵ E are independently as defined above (in particular with respect to formula (I), (Ia), (II), (III), (IV), (V), (Va) or (VI)) or below, and at least one of R^{4a} and R^{4b} is selected from the group consisting of C₁₋₈ alkyl, and —(CH₂)ₛ-[0-(CH₂)ₜ]ᵤ—H wherein s is an integer between 0 and 7, t is an integer between 0 and 7, u is an integer between 1 and 3, wherein if t is 0 then u is 1, and the total number of carbon and oxygen atoms of the —(CH₂)ₛ-[0-(CH₂)ₜ]ᵤ—H does not exceed 8. In a preferred of such embodiment, at least one of R^{4a} and R^{4b} is selected from the group consisting of C₁₋₃ alkyl, and C₂₋₃ alkyloxy, such as selected from the group consisting of Me, Eth and MeO.

In an alternative first aspect, and as may be further described, defined, claimed or otherwise disclosed herein, the present invention provides a compound selected from the group consisting of a kinase inhibitor of the formula (VIIa):

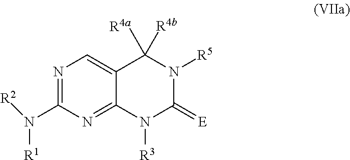

wherein R¹, R², R³, R⁵ E are independently as defined above (in particular with respect to formula (I), (Ia), (II), (III), (IV), (V), (Va) or (VI)) or below, and R^{4a} and R^{4b} together with the carbon to which they are attached form C═O.

In one embodiment, the kinase inhibitor has the formula (VIII):

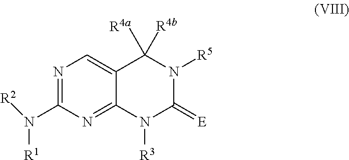

wherein R¹, R², R³, R^{4a}, R^{4b}, R⁵ E are independently as defined above (in particular with respect to formula (I), (Ia), (II), (III), (IV), (V), (Va), (VI), (VII) or (VIIa)) or below, R¹ and R³ join together via a group L′ to form a moiety R¹-L′-R³, preferably to form a moiety Q-L′-R³, and L′ is selected from the group consisting of C₃₋₁₀ alkylene, C₃₋₁₀ alkenylene, C₃₋₁₀ alkynylene, —(CH₂)ₚ—[Y—(CH₂)_{q}]ᵣ—, and alkenylene-[Y—(CH₂)_{q}]ᵣ—, wherein p is an integer between 1 and 10, q is an integer between 0 and 6, r is an integer between 1 and 3, wherein if q is 0 then r is 1; Y is independently selected from 0, S, and —N(R¹³)—; and each of the C₃₋₁₀ alkylene, C₃₋₁₀ alkenylene, C₃₋₁₀ alkynylene, —(CH₂)ₚ-, and —(CH₂)_{q}— groups is optionally substituted with one or more independently selected R³⁰.

In one embodiment of the kinase inhibitor has the formula (VIII) R¹, R², R³, R^{4a}, R^{4b}, R⁵ E are independently as defined above (in particular with respect to formula (I), (Ia), (II), (III), (IV), (V), (Va), (VI), (VII) or (VIIa)) or below, R¹ and R³ join together via a group L′ to form a moiety R¹-L′-R³, preferably to form a moiety Q-L′-R³, and L′ is selected from the group consisting of methylene, ethylene (i.e., 1,1-ethylene, 1,2-ethylene), propylene (i.e., 1,1-propylene, 1,2-propylene (—CH(CH₃)CH₂—), 2,2-propylene (—C(CH₃)₂—), and 1,3-propylene), the butylene isomers (e.g., 1,1-butylene, 1,2-butylene, 2,2-butylene, 1,3-butylene, 2,3-butylene (cis or trans or a mixture thereof), 1,4-butylene, 1,1-iso-butylene, 1,2-iso-butylene, and 1,3-iso-butylene), the pentylene isomers (e.g., 1,1-pentylene, 1,2-pentylene, 1,3-pentylene, 1,4-pentylene, 1,5-pentylene, 1,1-iso-pentylene, 1,1-sec-pentyl, 1,1-neo-pentyl), the hexylene isomers (e.g., 1,1-hexylene, 1,2-hexylene, 1,3-hexylene, 1,4-hexylene, 1,5-hexylene, 1,6-hexylene, and 1,1-isohexylene), the heptylene isomers (e.g., 1,1-heptylene, 1,2-heptylene, 1,3-heptylene, 1,4-heptylene, 1,5-heptylene, 1,6-heptylene, 1,7-heptylene, and 1,1-isoheptylene), the octylene isomers (e.g., 1,1-octylene, 1,2-octylene, 1,3-octylene, 1,4-octylene, 1,5-octylene, 1,6-octylene, 1,7-octylene, 1,8-octylene, and 1,1-isooctylene, vinylidene (also called ethenylidene), 1-propen-1,2-diyl, 1-propen-1,3-diyl, 1-propen-2,3-diyl, allylidene, 1-buten-1,2-diyl, 1-buten-1,3-diyl, 1-buten-1,4-diyl, 1-buten-2,3-diyl, 1-buten-2,4-diyl, 1-buten-3,4-diyl, 2-buten-1,2-diyl, 2-buten-1,3-diyl, 2-buten-1,4-diyl, 2-buten-2,3-diyl, 2-buten-2,4-diyl, 2-buten-3,4-diyl, vinylidene-[Y—(CH₂)_{q}]r-, 1-propen-1,2-diy-[Y—(CH₂)_{q}]ᵣ—, 1-propen-1,3-diyl-[Y—(CH₂)_{q}]ᵣ—, 1-propen-2,3-diyl-[Y—(CH₂)_{q}]ᵣ—, allylidene-[Y—(CH₂)_{q}]ᵣ—, 1-buten-1,2-diyl-[Y—(CH₂)_{q}]ᵣ—, 1-buten-1,3-diyl-[Y—(CH₂)_{q}]ᵣ—, 1-buten-1,4-diyl-[Y—(CH₂)_{q}]ᵣ—, 1-buten-2,3-diyl-[Y—(CH₂)_{q}]ᵣ—, 1-buten-2,4-diyl-[Y—(CH₂)_{q}]ᵣ—, 1-buten-3,4-diyl-[Y—(CH₂)_{q}]ᵣ—, 2-buten-1,2-diyl-[Y—(CH₂)_{q}]ᵣ—, 2-buten-1,3-diyl-[Y—(CH₂)_{q}]ᵣ—, 2-buten-1,4-diyl-[Y—(CH₂)_{q}]ᵣ—, 2-buten-2,3-diyl-[Y—(CH₂)_{q}]ᵣ—, 2-buten-2,4-diyl-[Y—(CH₂)_{q}]ᵣ—, and 2-buten-3,4-diyl-[Y—(CH₂)_{q}]ᵣ—.

In one embodiment of the kinase inhibitor has the formula (VIII) R¹, R², R³, R^{4a}, R^{4b}, R⁵ E are independently as defined above (in particular with respect to formula (I), (Ia), (II), (III), (IV), (V), (Va), (VI), (VII) or (VIIa)) or below, R¹ and R³ join together via a group L′ to form a moiety R¹-L′-R³, preferably to form a moiety Q-L′-R³, and L′ is selected from the group consisting of C₃₋₁₀ alkenylene, and C₃₋₁₀ alkenylene-[Y—(CH₂)_{q}]ᵣ—.

In one embodiment of the kinase inhibitor has the formula (VIII) R¹, R², R³, R^{4a}, R^{4b}, R⁵ E are independently as defined above (in particular with respect to formula (I), (Ia), (II), (III), (IV), (V), (Va), (VI), (VII) or (VIIa)) or below, R¹ and R³ join together via a group L′ to form a moiety R¹-L′-R³, preferably to form a moiety Q-L′-R³, and L′ is selected from the group consisting of C₃₋₁₀ alkenylene, and C₃₋₁₀ alkenylene —[O—(CH₂)_{q}]ᵣ—.

In one embodiment of the kinase inhibitor has the formula (VIII) R¹, R², R³, R^{4a}, R^{4b}, R⁵ E are independently as defined above (in particular with respect to formula (I), (Ia), (II), (III), (IV), (V), (Va), (VI), (VII) or (VIIa)) or below, R¹ and R³ join together via a group L′ to form a moiety R¹-L′-R³, preferably to form a moiety Q-L′-R³, and L′ is C₃₋₁₀ alkenylene-[O—(CH₂)_{q}]ᵣ—.

In one embodiment of the kinase inhibitor has the formula (VIII) R¹, R², R³, R^{4a}, R^{4b}, R⁵ E are independently as defined above (in particular with respect to formula (I), (Ia), (II), (III), (IV), (V), (Va), (VI), (VII) or (VIIa)) or below, R¹ and R³ join together via a group L′ to form a moiety R¹-L′-R³, preferably to form a moiety Q-L′-R³, and L′ is C₃₋₁₀ alkenylene-O—.

In one embodiment of the kinase inhibitor has the formula (VIII) R¹, R², R³, R^{4a}, R^{4b}, R⁵ E are independently as defined above (in particular with respect to formula (I), (Ia), (II), (III), (IV), (V), (Va), (VI), (VII) or (VIIa)) or below, R¹ and R³ join together via a group L′ to form a moiety R¹-L′-R³, preferably to form a moiety Q-L′-R³, and L′ is C₄₋₈ alkenylene-O—.

In one embodiment of the kinase inhibitor has the formula (VIII) R¹, R², R³, R^{4a}, R^{4b}, R⁵ E are independently as defined above (in particular with respect to formula (I), (Ia), (II), (III), (IV), (V), (Va), (VI), (VII) or (VIIa)) or below, R¹ and R³ join together via a group L′ to form a moiety R¹-L′-R³, preferably to form a moiety Q-L′-R³, and L′ is selected from the group consisting of

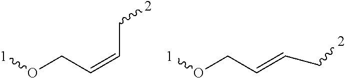

wherein 1 [symbol] represents the bond by which L′ is bound to R¹, and [symbol] 2 represents the bond by which L′ is bound to R³.

[symbol]

In any of the above embodiments of the kinase inhibitor of formula (VIII) R¹, R², R³, R^{4a}, R^{4b}, R⁵ E are independently as defined above (in particular with respect to formula (I), (Ia), (II), (III), (IV), (V), (Va), (VI), (VII) or (VIIa)) or below, R¹ and R³ join together via a group L′ to form a moiety R¹-L′-R³, preferably to form a moiety Q-L′-R³, and L′ is selected from the group consisting of,

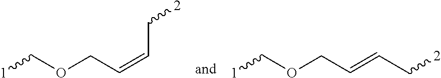

wherein 1 [symbol] represents the bond by which L′ is bound to R¹, and [symbol] 2 represents the bond by which L′ is bound to R³.

In any of the above embodiments of the kinase inhibitor of formula (VIII) R¹, R², R³, R^{4a}, R^{4b}, R⁵ E are independently as defined above (in particular with respect to formula (I), (Ia), (II), (III), (IV), (V), (Va), (VI), (VII) or (VIIa)) or below, R¹ and R³ join together via a group L′ to form a moiety R¹-L′-R³, preferably to form a moiety Q-L′-R³, and: (i) R³ is a bond; and/or (ii) Q is C₆₋₁₀ aryl (preferably C₆ aryl), wherein the C₆₋₁₀ aryl is, preferably, not substituted with one or more R³⁰ being —OR¹¹, wherein R¹¹ is independently selected from C₁₋₈ alkyl.

In embodiments of the kinase inhibitor of formula (I), (Ia), (II), (III), (IV), (V), (Va), (VI), (VII), (VIIa) or (VIII), when L′ is present, the moiety R¹-L′-R³ (for example, the moiety Q-L′-R³) comprises a (eg, 3- to 14-membered, such as a 3- to 10-membered) cycloalkyl or heterocyclyl, preferably 3- to 10-membered mono- or bi-cyclic cycloalkyl, where such cycloalkyl or heterocyclyl is optionally substituted with one or more R³⁰. In certain of such embodiments, the cycloalkyl is 5- or 6-membered mono-cyclic or a 7-membered bi-cyclic cycloalkyl, such as cyclopentyl, cyclohexyl or bi-cyclic heptyl, in each case where such cycloalkyl is optionally substituted with one two or three (preferably one) R³⁰. Suitable such cycloalkyls are disclosed in WO2010028116, in particular those disclosed in the examples thereof.

In any of the above embodiments of the kinase inhibitor of formula (I), (Ia), (II), (III), (IV), (V), (Va), (VI), (VII), (VIIa) or (VIII), L may be selected from the group consisting of a bond, C₁₋₆ alkylene, C₂₋₆ alkenylene, C₂₋₆ alkynylene, and —(CH₂)ₘ[Y—(CH₂)ₙ]o-, wherein m is 1, 2, or 3, n is 0, 1, or 2, o is 1, 2, or 3, wherein if n is 0 then o is 1; Y is independently selected from 0, S, and NH, wherein each of the C₁₋₆ alkylene, C₂₋₆ alkenylene, C₂₋₆ alkynylene, —(CH₂)ₘ—, and —(CH₂)ₙ— groups is optionally substituted with one or more, such as with one or two, independently selected R³⁰. For example, in any of the above embodiments of the kinase inhibitor of formula (I), (Ia), (II), (III), (IV), (V), (Va), (VI), (VII), (VIIa) or (VIII), L may be selected from the group consisting of a bond; C₁ alkylene, optionally substituted with one R³⁰; C₂ alkylene (in particular 1,2-ethylene or 1,1-ethylene), optionally substituted with one R³⁰; C₃ alkylene (in particular trimethylene), optionally substituted with one R³⁰; C₄ alkylene (in particular tetramethylene or 2,4-butandiyl), optionally substituted with one R³⁰; —(CH₂)ₘO—; and —(CH₂)ₘNH—, wherein m is 1, 2, or 3. Particularly, in any of the above embodiments of the kinase inhibitor of formula (I), (Ia), (II), (III), (IV), (V), (Va), (VI), (VII), (VIIa) or (VIII)), L may be a bond (i.e., R⁵ is R⁶).

In any of the above embodiments of the kinase inhibitor of formula (I), (Ia), (III), (IV), (V), (Va), (VI), (VII), (VIIa) (VIII), (IX), (XII) and (XIII)), where R⁶ is a heterocyclyl or heteroaryl (e.g., a 5-membered heteroaryl) containing an N atom as ring heteroatom, L may be attached to R⁶ via the N ring atom of the heterocyclyl or heteroaryl group.

In one embodiment, the kinase inhibitor has the formula (IX):

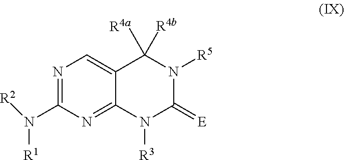

and
R¹ is -Q-R^{1a};
R^{1a} is selected from the group consisting of alkyl, —O(alkyl), —S(alkyl), —NH(alkyl), —N(alkyl)₂, and heterocyclyl, wherein each of the alkyl and heterocyclyl groups is optionally substituted with one or more independently selected R³⁰;
Q is selected from the group consisting of cycloalkyl, aryl, heterocyclyl, heteroaryl, wherein each of the cycloalkyl, aryl, heterocyclyl, and heteroaryl groups is optionally substituted with one or more independently selected R³⁰;
R² is H;
E is O;
R³ is selected from the group consisting of H, alkyl, alkenyl, alkynyl, cycloalkyl, aryl, heterocyclyl, heteroaryl, wherein each of the alkyl, alkenyl, alkynyl, cycloalkyl, aryl, heterocyclyl, and heteroaryl groups is optionally substituted with one or more independently selected R³⁰;
optionally R¹ and R³ may join together via a group L′ to form a moiety R¹-L′-R³, preferably to form a moiety Q-L′-R³;
R^{4a} and R^{4b} are independently selected from the group consisting of H, C₁₋₃ alkyl; or optionally R^{4a} and R^{4b} may join together to form, together with the carbon to which they are attached, C₃₋₆ cycloalkyl or a 4- to 6-membered heterocyclyl comprising at least one 0 as the only heteroatom element, wherein in case that the 4- to 6-membered heterocyclyl comprises more than one 0, different 0 are not directly bound to each other, preferably wherein R^{4a} and R^{4b} are independently selected from the group consisting of H, and methyl; or optionally R^{4a} and R^{4b} may join together to form, together with the carbon to which they are attached, cyclopropyl, most preferably wherein both R^{4a} and R^{4b} are H;
R⁵ is -L-R⁶;
R⁶ is a mono- or bicyclic heteroaryl or a mono- or bicyclic heterocyclyl, each of which is optionally substituted with one, two, or three independently selected R⁷, preferably wherein R⁶ is a 5-membered monocyclic heteroaryl which contains at least one S ring atom and which is substituted with one or more independently selected R⁷;
R⁷ is independently selected from the group consisting of C₁₋₃ alkyl, halogen, —CN, —O(C₁₋₃ alkyl), —NH(C₁₋₃ alkyl), and —N(C₁₋₃ alkyl)₂, and/or any two R⁷ which are bound to the same atom of R⁶ being a heterocyclyl group may join together to form ═O, wherein each of the C₁₋₃ alkyl groups is optionally substituted with one or more independently selected R³⁰;
L is a bond; and
L′, if present, is selected from the group consisting of C₃₋₁₀ alkylene, C₃₋₁₀ alkenylene, C₃₋₁₀ alkynylene, —(CH₂)ₚ-[Y—(CH₂)_{q}]ᵣ, and alkenylene-[Y—(CH₂)_{q}]ᵣ—, wherein p is an integer between 1 and 10, q is an integer between 0 and 6, r is an integer between 1 and 3, wherein if q is 0 then r is 1; Y is independently selected from O, S, and —N(R¹³)—; and each of the C₃₋₁₀ alkylene, C₃₋₁₀ alkenylene, C₃₋₁₀ alkynylene, —(CH₂)ₚ-, and —(CH₂)_{q}— groups is optionally substituted with one or more independently selected R³⁰,

In one embodiment, the kinase inhibitor has the formula (X):

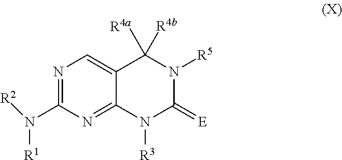

and
R¹ is -Q-R^{1a};
R^{1a} is selected from the group consisting of piperidinyl substituted with one to three moieties independently selected from the group consisting of C₁ to C₄ alkyl; piperazinyl group substituted with one or two moieties independently selected from the group consisting of 2-hydroxyethyl and C₁ to C₄ alkyl; azepanyl substituted with one to three moieties independently selected from the group consisting of C₁ to C₄ alkyl; morpholinyl; and C₁₋₃ alkyl group substituted with —NH_{2-z}(CH₃)_{z}, wherein z is 0, 1, or 2;
Q is C₆₋₁₀ aryl, wherein the C₆₋₁₀ aryl is optionally substituted with one or more R³⁰ being —OR¹¹, wherein R¹¹ is independently selected from C₁₋₁₂ alkyl;
R² is H;
E is O;
R³ is selected from the group consisting of C₁₋₆ alkyl, C₆ aryl, and 5- or 6-membered heteroaryl, wherein each of the C₁₋₆ alkyl, C₆ aryl, and 5- or 6-membered heteroaryl groups is optionally substituted with one or more R³⁰ independently selected from the group consisting of C₁₋₆ alkyl, and OR¹¹, wherein R¹¹ C₁₋₁₂ alkyl;
   optionally R¹ and R³ may join together via a group L′ to form a moiety R¹-L′-R³, preferably to form a moiety Q-L′-R³;
R^{4a} and R^{4b} are independently selected from the group consisting of H, and methyl; or optionally R^{4a} and R^{4b} may join together to form, together with the carbon to which they are attached, cyclopropyl, preferably wherein both R^{4a} and R^{4b} are H;
R⁵ is -L-R⁶;
R⁶ is thienyl substituted with one, two, or three independently selected R⁷;
R⁷ is independently selected from the group consisting of Cl, F, methyl, fluoromethyl, difluoromethyl, and trifluoromethyl, preferably wherein at least one of R⁷ is F and/or at least one of R⁷ is substituted with one or more F atoms;
L is a bond; and
L′, if present, is C₃₋₁₀ alkenylene-[O—(CH₂)_{q}]ᵣ—.

In one embodiment, the kinase inhibitor has the formula (XI):

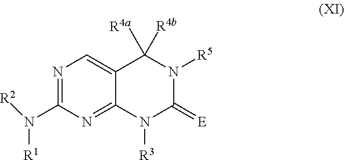

and
R¹ is -Q-R^{1a};
R^{1a} is selected from the group consisting of alkyl, —O(alkyl), —S(alkyl), —NH(alkyl), —N(alkyl)₂, and heterocyclyl, wherein each of the alkyl and heterocyclyl groups is optionally substituted with one or more independently selected R³⁰;
Q is selected from the group consisting of cycloalkyl, aryl, heterocyclyl, heteroaryl, wherein each of the cycloalkyl, aryl, heterocyclyl, and heteroaryl groups is optionally substituted with one or more independently selected R³⁰;
R² is H;
E is O;
R³ is selected from the group consisting of H, alkyl, alkenyl, alkynyl, cycloalkyl, aryl, heterocyclyl, heteroaryl, wherein each of the alkyl, alkenyl, alkynyl, cycloalkyl, aryl, heterocyclyl, and heteroaryl groups is optionally substituted with one or more independently selected R³⁰;
   optionally R¹ and R³ may join together via a group L′ to form a moiety R¹-L′-R³, preferably to form a moiety Q-L′-R³;
both R^{4a} and R^{4b} are H;
R⁵ is -L-R⁶;
R⁶ is selected from the group consisting of

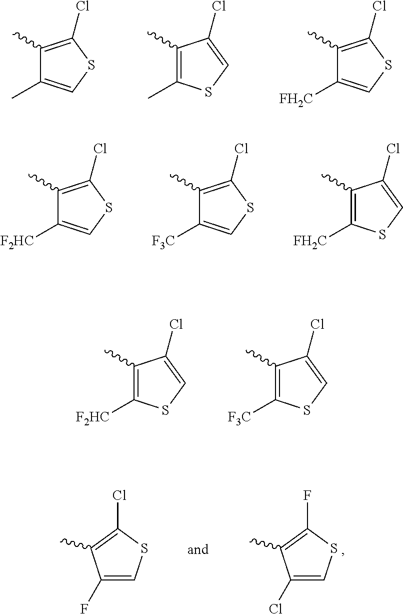

wherein [symbol] represents the bond by which R⁶ is bound to the remainder of the compound;
L is a bond; and
L′, if present, is selected from the group consisting of C₃₋₁₀ alkylene, C₃₋₁₀ alkenylene, C₃₋₁₀ alkynylene, —(CH₂)ₚ-[Y—(CH₂)_{q}]ᵣ, and alkenylene-[Y—(CH₂)_{q}]ᵣ—, wherein p is an integer between 1 and 10, q is an integer between 0 and 6, r is an integer between 1 and 3, wherein if q is 0 then r is 1; Y is independently selected from 0, S, and —N(R¹³)—; and each of the C₃₋₁₀ alkylene, C₃₋₁₀ alkenylene, C₃₋₁₀ alkynylene, —(CH₂)ₚ-, and —(CH₂)_{q}— groups is optionally substituted with one or more independently selected R³⁰.

In one embodiment, the kinase inhibitor has the formula (XII):

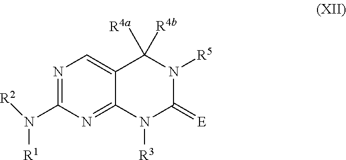

and
R¹ is -Q-R^{1a};
R^{1a} is selected from the group consisting of 1-methylpiperidinyl, 1,2-dimethylpiperidinyl, 1,2,6-trimethylpiperidinyl, 1-methylazepanyl, 4-(2-hydroxyethyl)piperazinyl, 1-methyl-4-(2-hydroxyethyl)piperazinyl, 4-methylpiperazinyl, 4-acetylpiperazinyl, and (2-hydroxyethyl)amino;
Q is C₆ aryl, wherein the C₆ aryl is optionally substituted with one or more —OR¹¹, wherein R¹¹ is methyl;
R² is H;
E is O;
R³ is selected from the group consisting of methyl, 2-methoxyethyl, methoxyphenyl, and 3-methoxypyridyl, and 1,3-dimethoxyphenyl;
   optionally R¹ and R³ may join together via a group L′ to form a moiety R¹-L′-R³, preferably to form a moiety Q-L′-R³;
both R^{4a} and R^{4b} are H;
R⁵ is -L-R⁶;
R⁶ is selected from the group consisting of

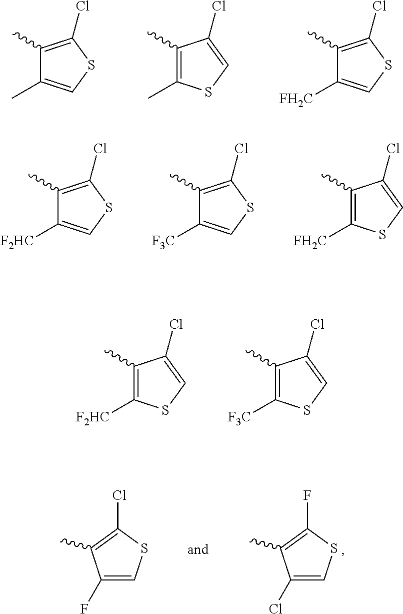

wherein [symbol] represents the bond by which R⁶ is bound to the remainder of the compound;
L is a bond; and [symbol]
L′, if present, is selected from the group consisting of

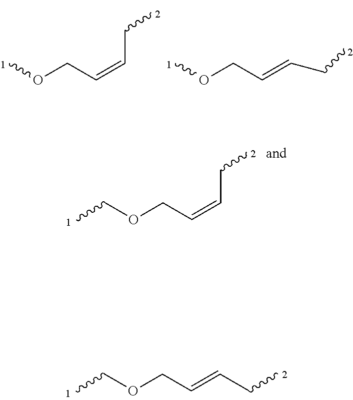

wherein 1 [symbol] represents the bond by which L′ is bound to R¹, and [symbol] 2 represents the bond by which L′ is bound to R³.

[symbol]

In particular of such embodiments, in the kinase inhibitor of formula (IX), (X), (XII) or (XII);

(a) R⁶ is selected from the group consisting of:

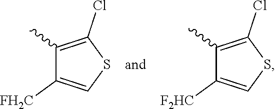

wherein [symbol] represents the bond by which R⁶ is bound to the remainder of the compound; and/or
(B) (i) Q is C₆ aryl, wherein the C₆ aryl is optionally substituted with one or more R³⁰ being —OR¹¹, wherein R¹¹ is independently selected from C₁₋₄alkyl; and (ii) R^{1a} is selected from the group consisting of:

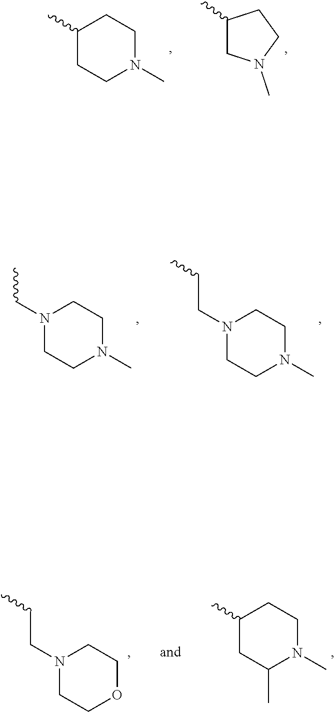

wherein [symbol] represents the bond by which R^{1a} is bound to the remainder of the compound.

In particular of such embodiments, in the kinase inhibitor of formula (IX), (X), (XII) or (XII)), R¹ and R³ join together via a group L′ to form a moiety R¹-L′-R³, preferably to form a moiety Q-L′-R³. In particular of such embodiments, L′ is selected from the group consisting of

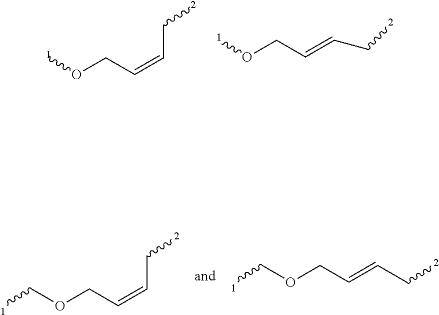

wherein 1 [symbol] represents the bond by which L′ is bound to R¹, and [symbol] 2 represents the bond by which L′ is bound to R³.

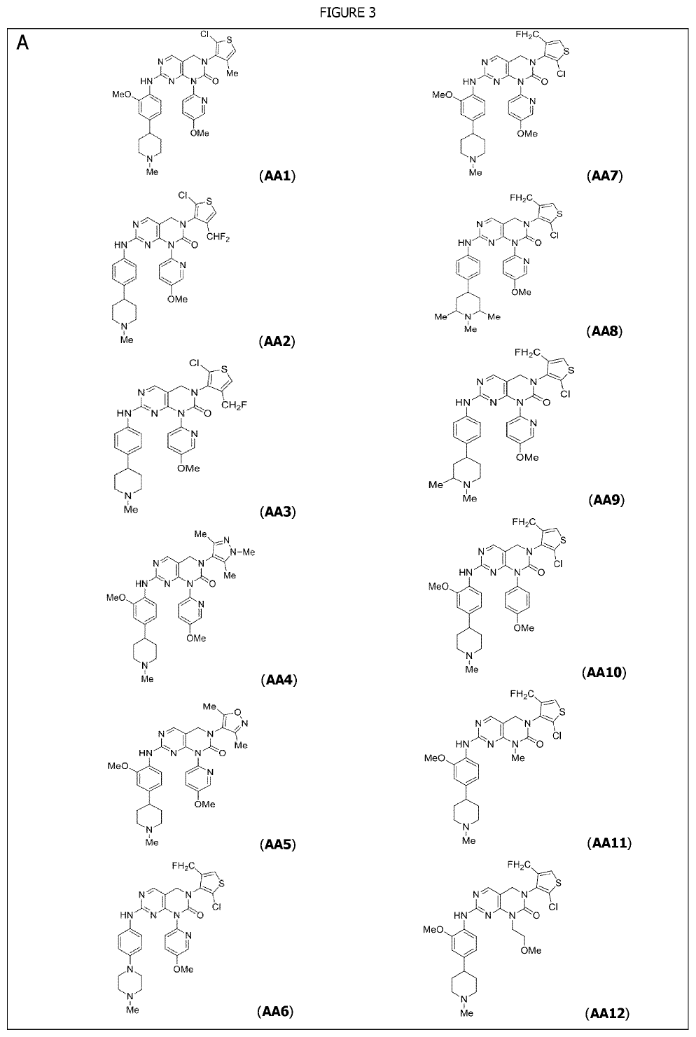

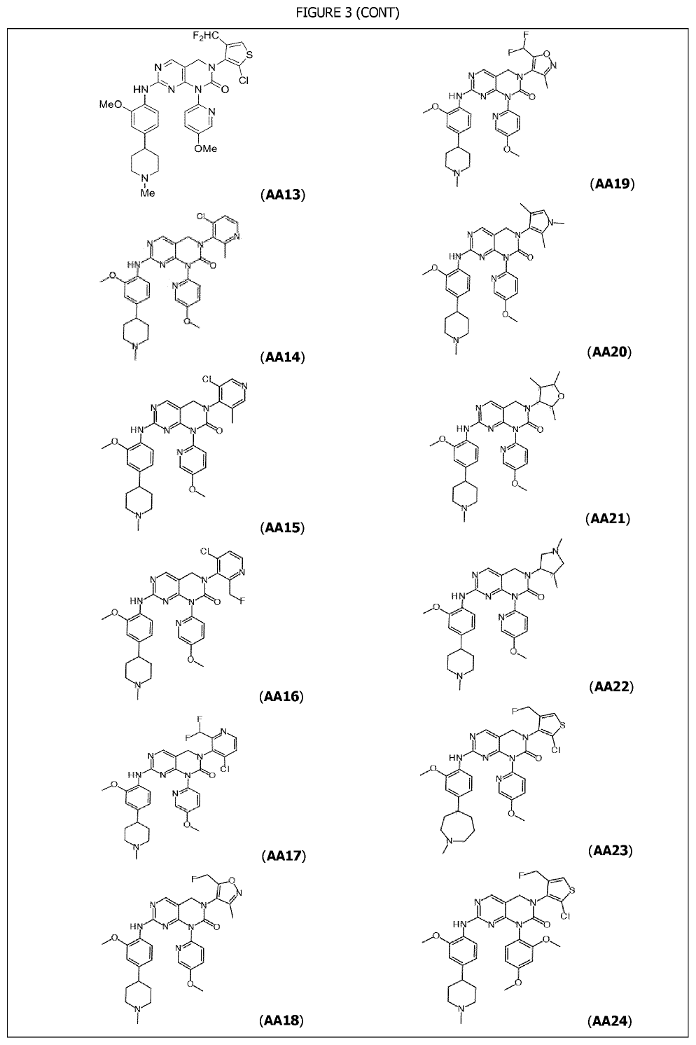

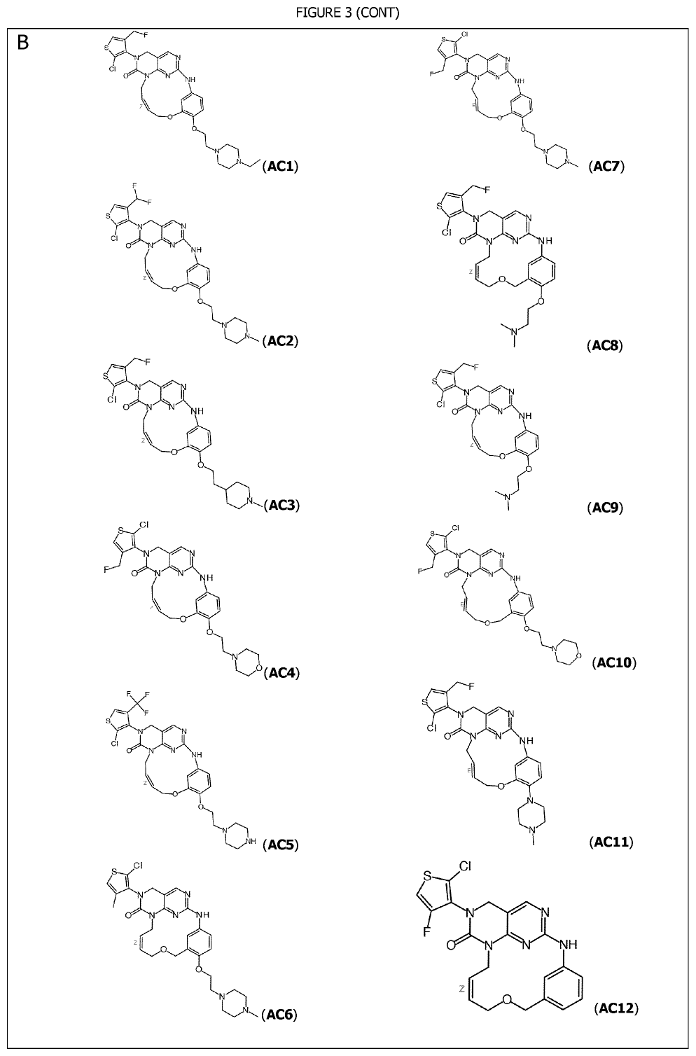

In one embodiment, the compound of the invention is selected from the compounds shown in Table A and Table B and/or those depicted in FIG. 3 A and/or B.

It is intended that the compounds of the present invention (in particular, the compounds of any one of formulas (I), (Ia), (II), (III), (IV), (V), (Va), (VI), (VII), (VIIa), (VIII), (IX), (X), (XII) or (XII) such as those shown in Table A and Table B, below; and/or depicted in FIGS. 3.2 A and B) encompass not only the compounds as depicted but also their solvates, salts (in particular, pharmaceutically acceptable salts), N-oxides (in particular, N-oxides of R^{1a} and/or R^{6″}), complexes, polymorphs, crystalline forms, non-crystalline forms, amorphous forms, racemic mixtures, non-racemic mixtures, diastereomers, enantiomers, tautomers, conformers, isotopically labeled forms, prodrugs, and any combinations thereof.

A selection of compounds, including those which have been synthesised and tested, within the scope of, or for use within the methods of, the present invention—and/or that represent examples of various exemplary or preferred R¹ substituents (such as R^{1a} substituents and/or Q substituents), R² substituents, R³ substituents, R⁴ substituents, R⁵ moieties (such as L moieties, R⁶ substituents and/or R⁷ substituents), E moieties and/or L′ moieties, each individually or in any combination are useful for synthesising further compounds of the invention—is listed in the following Table A and/or Table B.

TABLE A
Kinase inhibitors of formula (1).

| Compound | | |
|---|---|---|
| Number | Structure | Name |
| AA1 | 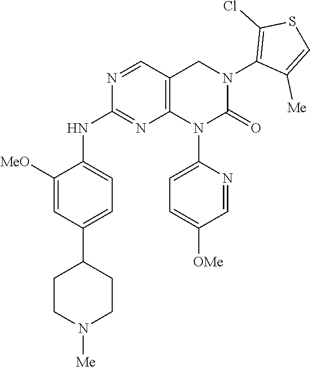 [SMILES: COc1ccc(N2C(=O)N(c3c(C)csc3Cl)Cc3cnc(Nc4ccc(C5CCN(C)CC5)cc4OC)nc32)nc1] | 3-(2-chloro-4-methylthiophen-3-yl)-7-((2-methoxy-4-(1- methylpiperidin-4-yl)phenyl)amino)-l-(5-methoxypyridin-2-yl)- 3,4-dihydropyrimido[4,5-d]pyrimidin-2(1H)-one |
| AA2 | 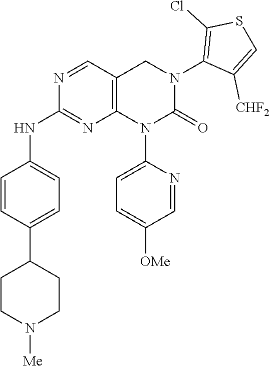 [SMILES: COc1ccc(N2C(=O)N(c3c(C(F)F)csc3Cl)Cc3cnc(Nc4ccc(C5CCN(C)CC5)cc4)nc32)nc1] | 3-(2-chloro-4-(difluoromethyl)thiophen-3-yl)-l-(5- methoxypyridin-2-yl)-7-((4-(l-methylpiperidin-4- yl)phenyl)amino)-3,4-dihydropyrimido[4,5-d]pyrimidin-2(1H)- one |
| AA3 | 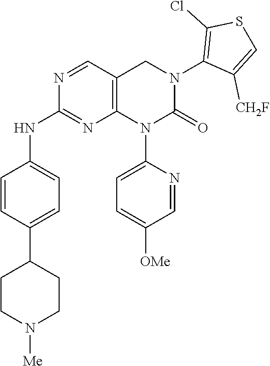 | 3-(2-chloro-4-(fluoromethyl)thiophen-3-yl)-l-(5- methoxypyridin-2-yl)-7-((4-(l-methylpiperidin-4- yl)phenyl)amino)-3,4-dihydropyrimido[4,5-d]pyrimidin-2(1H)- one |
| AA4 | 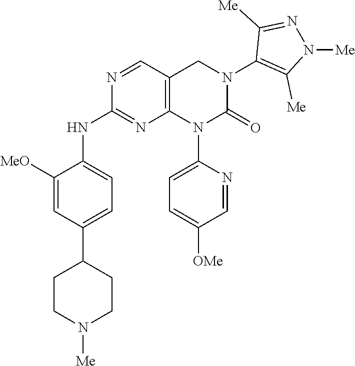 [SMILES: COc1ccc(N2C(=O)N(c3c(C)nn(C)c3C)Cc3cnc(Nc4ccc(C5CCN(C)CC5)cc4OC)nc32)nc1] | 7-((2-methoxy-4-(l-methylpiperidin-4-yl)phenyl)amino)-1-(5- methoxypyridin-2-yl)-3-(1,3,5-trimethyl-lH-pyrazol-4-yl)-3,4- dihydropyrimido[4,5-d]pyrimidin-2(1H)-one |
| AA5 | 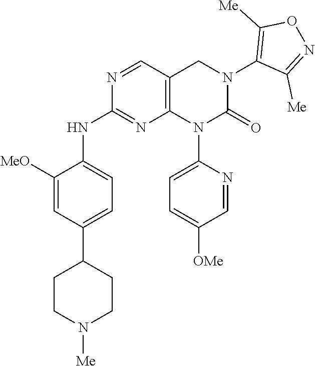 [SMILES: COc1ccc(N2C(=O)N(c3c(C)noc3C)Cc3cnc(Nc4ccc(C5CCN(C)CC5)cc4OC)nc32)nc1] | 3-(3,5-dimethylisoxazol-4-yl)-7-((2-methoxy-4-(1- methylpiperidin-4-yl)phenyl)amino)-1-(5-methoxypyridin-2-yl)- 3,4-dihydropyrimido[4,5-d]pyrimidin-2(1H)-one |
| AA6 | 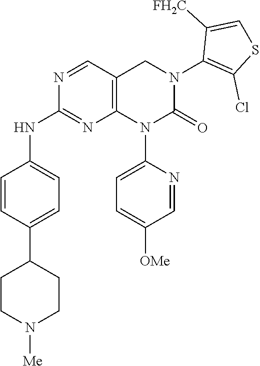 | 3-(2-chloro-4-(fluoromethyl)thiophen-3-yl)-1-(5- methoxypyridin-2-yl)-7-((4-(4-methylpiperazin-1- yl)phenyl)amino)-3,4-dihydropyrimido[4,5-d]pyrimidin-2(l H)- one |
| AA7 | 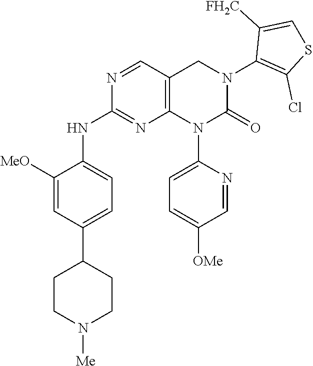 | 3-(2-chloro-4-(fluoromethyl)thiophen-3-yl)-7-((2-methoxy-4- (l-methylpiperidin-4-yl)phenyl)amino)-1-(5-methoxypyridin-2- yl)-3,4-dihydropyrimido[4,5-d]pyrimidin-2(1H)-one |
| AA8 | 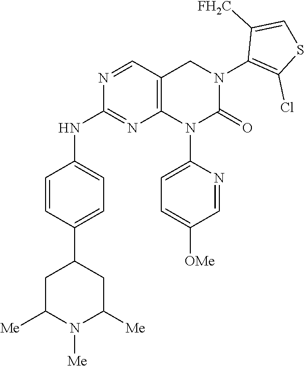 | 3-(2-chloro-4-(fluoromethyl)thiophen-3-yl)-1-(5- methoxypyridin-2-yl)-7-((4-(l,2,6-trimethylpiperidin-4- yl)phenyl)amino)-3,4-dihydropyrimido[4,5-d]pyrimidin-2(lH)- one |
| AA9 | 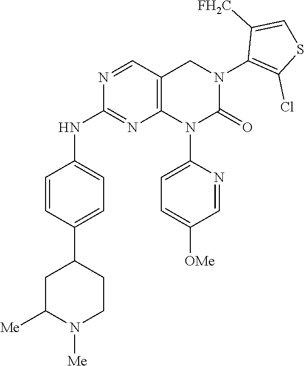 | 3-(2-chloro-4-(fluoromethyl)thiophen-3-yl)-7-((4-(l,2- dimethylpiperidin-4-yl)phenyl)amino)-1-(5-methoxypyridin-2- yl)-3,4-dihydropyrimido[4,5-d]pyrimidin-2(1H)-one |
| AA10 | 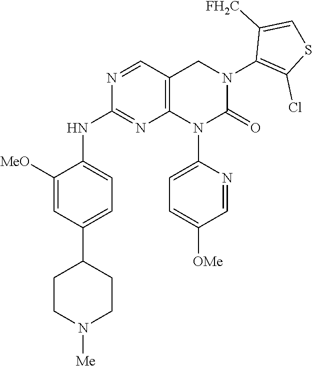 | 3-(2-chloro-4-(fluoromethyl)thiophen-3-yl)-7-((2-methoxy-4- (1-methylpiperidin-4-yl)phenyl)amino)-1-(4-methoxyphenyl)- 3,4-dihydropyrimido[4,5-d]pyrimidin-2(1H)-one |
| AA11 | 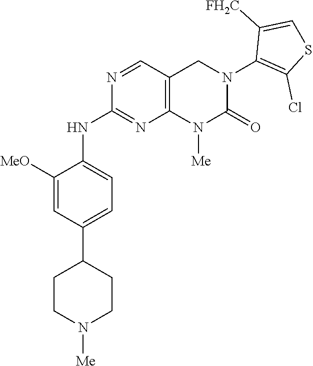 | 3-(2-chloro-4-(fluoromethyl)thiophen-3-yl)-7-((2-methoxy-4- (1-methylpiperidin-4-yl)phenyl)amino)-1-methyl-3,4- dihydropyrimido[4,5-d]pyrimidin-2(1H)-one |
| AA12 | 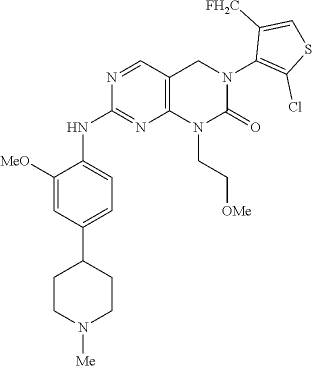 | 3-(2-chloro-4-(fluoromethyl)thiophen-3-yl)-7-((2-methoxy-4- (1-methylpiperidin-4-yl)phenyl)amino)-1-(2-methoxyethyl)- 3,4-dihydropyrimido[4,5-d]pyrimidin-2(1H)-one |
| AA13 | 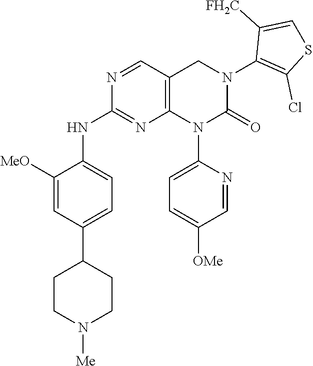 | 3-(2-chloro-4-(difluoromethyl)thiophen-3-yl)-7-((2-methoxy-4- (1-methylpiperidin-4-yl)phenyl)amino)-1-(5-methoxypyridin-2- yl)-3,4-dihydropyrimido[4,5-d]pyrimidin-2(1H)-one |
| AA14 | 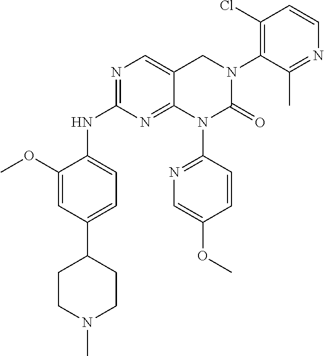 [SMILES: COc1ccc(N2C(=O)N(c3c(Cl)ccnc3C)Cc3cnc(Nc4ccc(C5CCN(C)CC5)cc4OC)nc32)nc1] | 3-(4-chloro-2-methylpyridin-3-yl)-7-((2-methoxy-4-(1- methylpiperidin-4-yl)phenyl)amino)-1-(5-methoxypyridin-2-yl)- 3,4-dihydropyrimido[4,5-d]pyrimidin-2(1H)-one |
| AA15 | 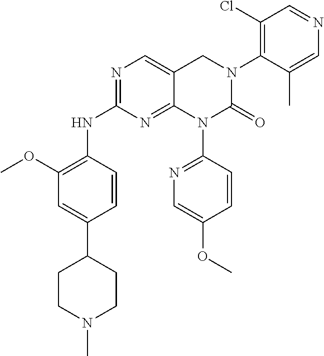 [SMILES: COc1ccc(N2C(=O)N(c3c(C)cncc3Cl)Cc3cnc(Nc4ccc(C5CCN(C)CC5)cc4OC)nc32)nc1] | 3-(3-chloro-5-methylpyridin-4-yl)-7-((2-methoxy-4-(1- methylpiperidin-4-yl)phenyl)amino)-1-(5-methoxypyridin-2-yl)- 3,4-dihydropyrimido[4,5-d]pyrimidin-2(1H)-one |
| AA16 | 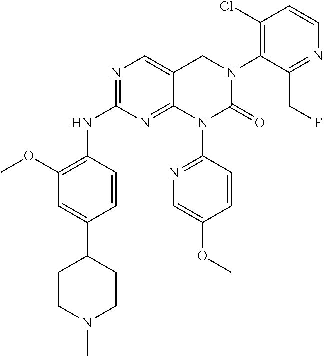 [SMILES: COc1ccc(N2C(=O)N(c3c(Cl)ccnc3CF)Cc3cnc(Nc4ccc(C5CCN(C)CC5)cc4OC)nc32)nc1] | 3-(4-chloro-2-(fluoromethyl)pyridin-3-yl)-7-((2-methoxy-4-(1- methylpiperidin-4-yl)phenyl)amino)-1-(5-methoxypyridin-2-yl)- 3,4-dihydropyrimido[4,5-d]pyrimidin-2(1H)-one |
| AA17 | 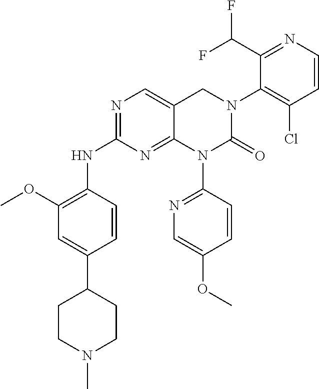 [SMILES: COc1ccc(N2C(=O)N(c3c(Cl)ccnc3C(F)F)Cc3cnc(Nc4ccc(C5CCN(C)CC5)cc4OC)nc32)nc1] | 3-(4-chloro-2-(difluoromethyl)pyridin-3-yl)-7-((2-methoxy-4- (1-methylpiperidin-4-yl)phenyl)amino)-1-(5-methoxypyridin-2- yl)-3,4-dihydropyrimido[4,5-d]pyrimidin-2(1H)-one |
| AA18 | 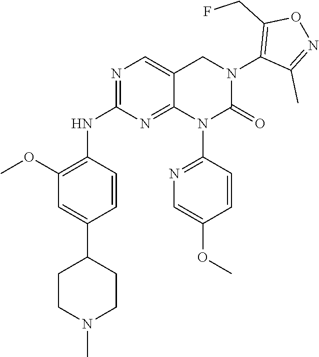 [SMILES: COc1ccc(N2C(=O)N(c3c(C)noc3CF)Cc3cnc(Nc4ccc(C5CCN(C)CC5)cc4OC)nc32)nc1] | 3-(5-(fluoromethyl)-3-methylisoxazol-4-yl)-7-((2-methoxy-4- (1-methylpiperidin-4-yl)phenyl)amino)-1-(5-methoxypyridin-2- yl)-3,4-dihydropyrimido[4,5-d]pyrimidin-2(1H)-one |
| AA19 | 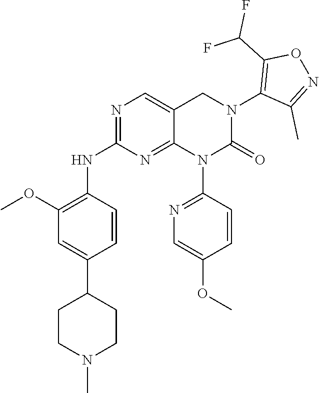 [SMILES: COc1ccc(N2C(=O)N(c3c(C)noc3C(F)F)Cc3cnc(Nc4ccc(C5CCN(C)CC5)cc4OC)nc32)nc1] | 3-(5-(difluoromethyl)-3-methylisoxazol-4-yl)-7-((2-methoxy-4- (1-methylpiperidin-4-yl)phenyl)amino)-1-(5-methoxypyridin-2- yl)-3,4-dihydropyrimido[4,5-d]pyrimidin-2(1H)-one |
| AA20 | 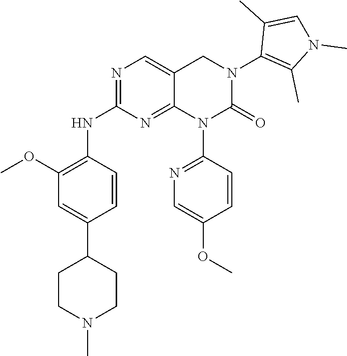 | 7-((2-methoxy-4-(1-methylpiperidin-4-yl)phenyl)amino)-1-(5- methoxypyridin-2-yl)-3-(1,2,4-trimethyl-1H-pyrrol-3-yl)-3,4- dihydropyrimido[4,5-d]pyrimidin-2(1H)-one |
| AA21 | 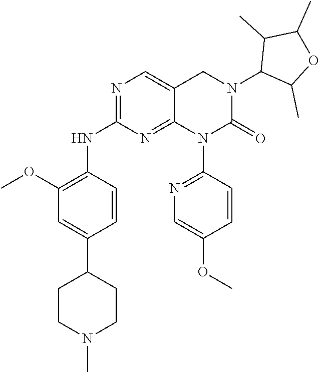 [SMILES: COc1ccc(N2C(=O)N(C3C(C)OC(C)C3C)Cc3cnc(Nc4ccc(C5CCN(C)CC5)cc4OC)nc32)nc1] | 7-((2-methoxy-4-(1-methylpiperidin-4-yl)phenyl)amino)-1-(5- methoxypyridin-2-yl)-3-(2,4,5-trimethyltetrahydrofuran-3-yl)- 3,4-dihydropyrimido[4,5-d]pyrimidin-2(1H)-one |
| AA22 | 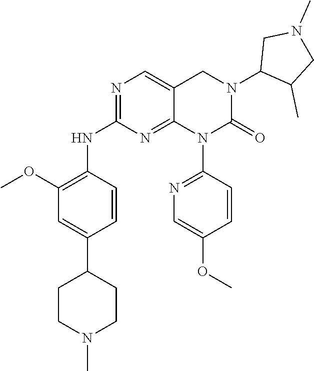 [SMILES: COc1ccc(N2C(=O)N(C3CN(C)CC3C)Cc3cnc(Nc4ccc(C5CCN(C)CC5)cc4OC)nc32)nc1] | 3-(1,4-dimethylpyrrolidin-3-yl)-7-((2-methoxy-4-(1- methylpiperidin-4-yl)phenyl)amino)-1-(5-methoxypyridin-2-yl)- 3,4-dihydropyrimido[4,5-d]pyrimidin-2(1H)-one |
| AA23 | 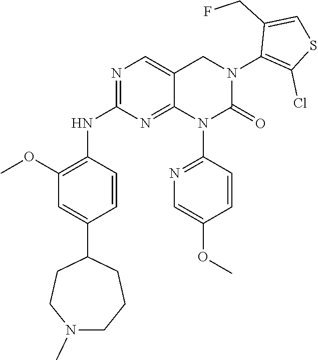 [SMILES: COc1ccc(N2C(=O)N(c3c(CF)csc3Cl)Cc3cnc(Nc4ccc(C5CCCN(C)CC5)cc4OC)nc32)nc1] | 3-(2-chloro-4-(fluoromethyl)thiophen-3-yl)-7-((2-methoxy-4- (1-methylazepan-4-yl)phenyl)amino)-1-(5-methoxypyridin-2- yl)-3,4-dihydropyrimido[4,5-d]pyrimidin-2(1H)-one |
| AA24 | 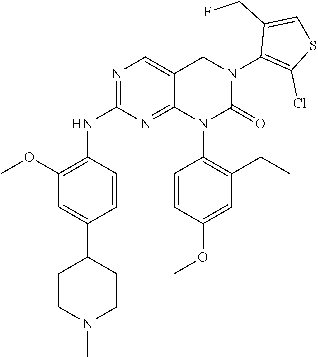 [SMILES: CCc1cc(OC)ccc1N1C(=O)N(c2c(CF)csc2Cl)Cc2cnc(Nc3ccc(C4CCN(C)CC4)cc3OC)nc21] | 3-(2-chloro-4-(fluoromethyl)thiophen-3-yl)-l-(2,4- dimethoxyphenyl)-7-((2-methoxy-4-(1-methylpiperidin-4- yl)phenyl)amino)-3,4-dihydropyrimido[4,5-d]pyrimidin-2(1H)- one |

TABLE B
Macrocyclic compounds of kinase inhibitors of formula (1).

| Compound | | |
|---|---|---|
| Number | Structure | Name |
| AC1 | 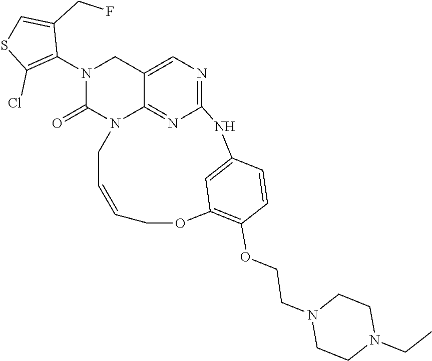 [SMILES: CCN1CCN(CCOc2ccc3cc2OC/C=C\CN2C(=O)N(c4c(CF)csc4Cl)Cc4cnc(nc42)N3)CC1] | (Z)-1³-(2-chloro-4-(fluoromethyl)thiophen-3-yl)-3⁴-(2-(4- ethylpiperazin-1-yl)ethoxy)-1¹,1²,1³,1⁴-tetrahydro-4-oxa-2- aza-1(7,1)-pyrimido[4,5-d]pyrimidina-3(1,3)- benzenacyclooctaphan-6-en-1²-one |
| AC2 | 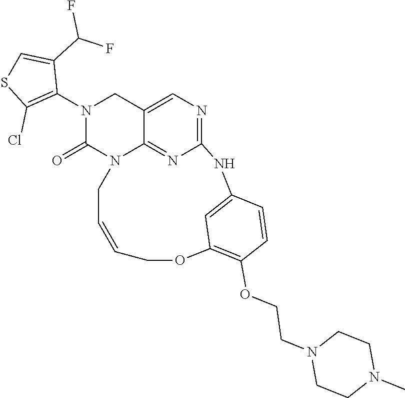 [SMILES: CN1CCN(CCOc2ccc3cc2OC/C=C\CN2C(=O)N(c4c(C(F)F)csc4Cl)Cc4cnc(nc42)N3)CC1] | (Z)-1³-(2-chloro-4-(difluoromethyl)thiophen-3-yl)-3⁴-(2-(4- methylpiperazin-l-yl)ethoxy)-1¹,1²,1³,1⁴-tetrahydro-4-oxa-2- aza-1(7,1)-pyrimido[4,5-d]pyrimidina-3(1,3)- benzenacyclooctaphan-6-en-1²-one |
| AC3 | 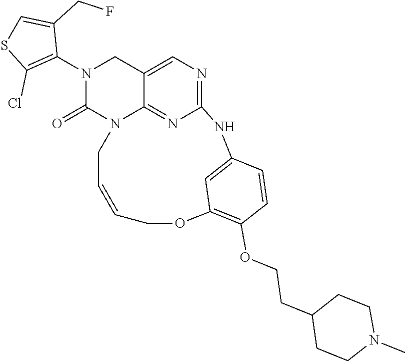 [SMILES: CN1CCC(CCOc2ccc3cc2OC/C=C\CN2C(=O)N(c4c(CF)csc4Cl)Cc4cnc(nc42)N3)CC1] | (Z)-1³-(2-chloro-4-(fluoromethyl)thiophen-3-yl)-3⁴-(2-(1- methylpiperidin-4-yl)ethoxy)-1¹,1²,1³,1⁴-tetrahydro-4-oxa-2- aza-1(7,1)-pyrimido[4,5-d]pyrimidina-3(1,3)- benzenacyclooctaphan-6-en-1²-one |
| AC4 | 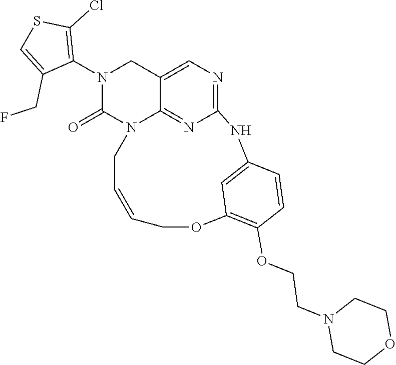 [SMILES: O=C1N2C/C=C\COc3cc(ccc3OCCN3CCOCC3)Nc3ncc(c2n3)CN1c1c(CF)csc1Cl] | (Z)-16-(2-chloro-4-(fluoromethyl)thiophen-3-yl)-7-(2- morpholinoethoxy)-3,4,5,6,7,8,10,13,16,17-decahydro-15H- 2,18-(azenometheno)-4,8-methanopyrimido[l,6- f\|[1]oxa[6,8,10]triazacyclopentadecin-15-one |
| AC5 | 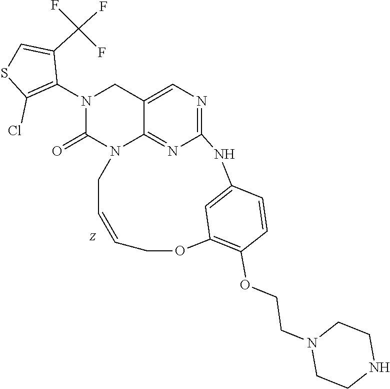 [SMILES: O=C1N2C/C=C\COc3cc(ccc3OCCN3CCNCC3)Nc3ncc(c2n3)CN1c1c(C(F)(F)F)csc1Cl] | (Z)-16-(2-chloro-4-(trifluoromethyl)thiophen-3-yl)-7-(2- (piperazin-l-yl)ethoxy)-3,4,5,6,7,8,10,13,16,17-decahydro- 15H-2,18-(azenometheno)-4,8-methanopyrimido[l,6- f\|[1]oxa[6,8,10]triazacyclopentadecin-15-one |
| AC6 | 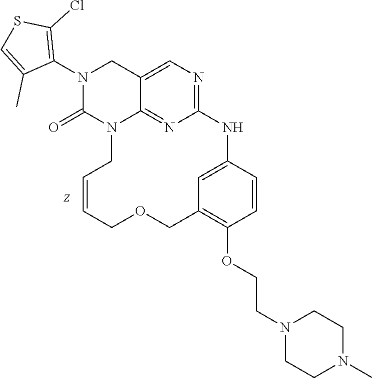 [SMILES: Cc1csc(Cl)c1N1Cc2cnc3nc2N(C/C=C\COCc2cc(ccc2OCCN2CCN(C)CC2)N3)C1=O] | (Z)-1³-(2-chloro-4-methylthiophen-3-yl)-3⁴-(2-(4- methylpiperazin-l-yl)ethoxy)-1¹,1²,1³,1⁴-tetrahydro-5-oxa-2- aza-1(7,1)-pyrimido[4,5-d]pyrimidina-3(1,3)- benzenacyclononaphan-7-en-1²-one |
| AC7 | 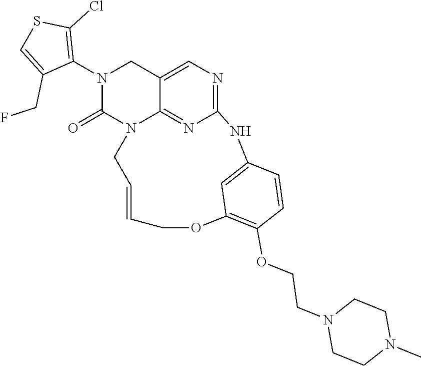 [SMILES: CN1CCN(CCOc2ccc3cc2OC/C=C/CN2C(=O)N(c4c(CF)csc4Cl)Cc4cnc(nc42)N3)CC1] | (Z)-1³-(2-chloro-4-(fluoromethyl)thiophen-3-yl)-3⁴-(2-(4- methylpiperazin-l-yl)ethoxy)-1¹,1²,1³,1⁴-tetrahydro-4-oxa-2- aza-1(7,1)-pyrimido[4,5-d]pyrimidina-3(1,3)- benzenacyclooctaphan-6-en-1²-one |
| AC8 | 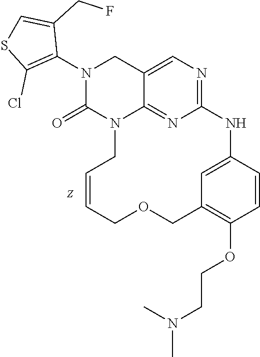 [SMILES: CN(C)CCOc1ccc2cc1COC/C=C\CN1C(=O)N(c3c(CF)csc3Cl)Cc3cnc(nc31)N2] | (Z)-1³-(2-chloro-4-(fluoromethyl)thiophen-3-yl)-3⁴-(2- (dimethylamino)ethoxy)-1¹,1²,1³,1⁴-tetrahydro-5-oxa-2-aza- 1(7,1)-pyrimido[4,5-d]pyrimidina-3(1,3)- benzenacyclononaphan-7-en-1²-one |
| AC9 | 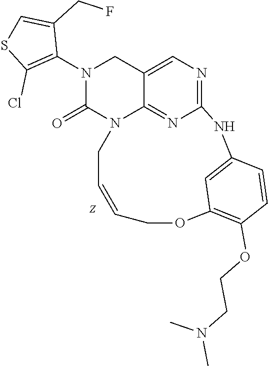 [SMILES: CN(C)CCOc1ccc2cc1OC/C=C\CN1C(=O)N(c3c(CF)csc3Cl)Cc3cnc(nc31)N2] | (Z)-1³-(2-chloro-4-(fluoromethyl)thiophen-3-yl)-3⁴-(2- (dimethylamino)ethoxy)-1¹,1²,1³,1⁴-tetrahydro-4-oxa-2-aza- 1(7,1)-pyrimido[4,5-d]pyrimidina-3(1,3)- benzenacyclooctaphan-6-en-1²-one |
| AC10 | 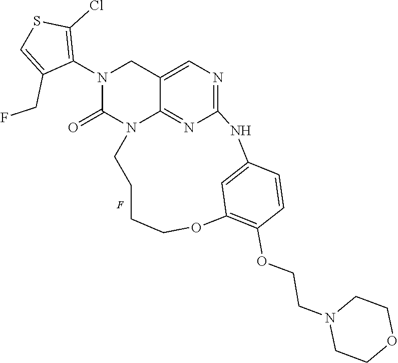 | (E)-1³-(2-chloro-4-(fluoromethyl)thiophen-3-yl)-3⁴-(2- morpholinoethoxy)-1¹,1²,1³,1⁴-tetrahydro-5-oxa-2-aza- 1(7,1)-pyrimido[4,5-d]pyrimidina-3(1,3)- benzenacyclononaphan-7-en-1²-one |
| AC11 | 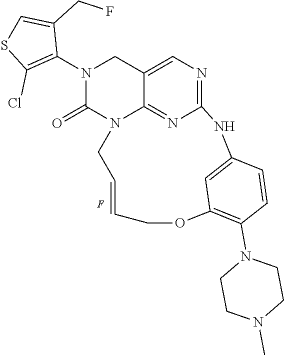 | (Z)-1³-(2-chloro-4-(fluoromethyl)thiophen-3-yl)-3⁴-(4- methylpiperazin-l-yl)-1¹,1²,1³,1⁴-tetrahydro-4-oxa-2-aza- 1(7,1)-pyrimido[4,5-d]pyrimidina-3(1,3)- benzenacyclooctaphan-6-en-1²-one |
| AC12 | 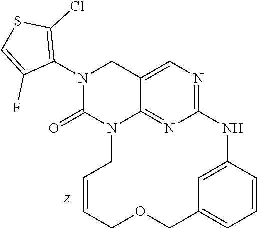 [SMILES: O=C1N2C/C=C\COCc3cccc(c3)Nc3ncc(c2n3)CN1c1c(F)csc1Cl] | (Z)-1³-(2-chloro-4-fluorothiophen-3-yl)-1¹,1²,1³,1⁴- tetrahydro-5-oxa-2-aza-l(7,1)-pyrimido[4,5-d]pyrimidina- 3(1,3)-benzenacyclononaphan-7-en-1²-one |
| AC13 | 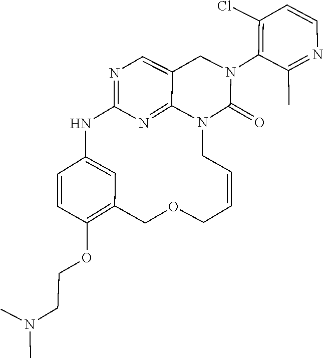 [SMILES: Cc1nccc(Cl)c1N1Cc2cnc3nc2N(C/C=C\COCc2cc(ccc2OCCN(C)C)N3)C1=O] | (Z)-1³-(4-chloro-2-methylpyridin-3-yl)-3⁴-(2- (dimethylamino)ethoxy)-1¹,1²,1³,1⁴-tetrahydro-5-oxa-2-aza- 1(7,1)-pyrimido[4,5-d]pyrimidina-3(1,3)- benzenacyclononaphan-7-en-1²-one |
| AC14 | 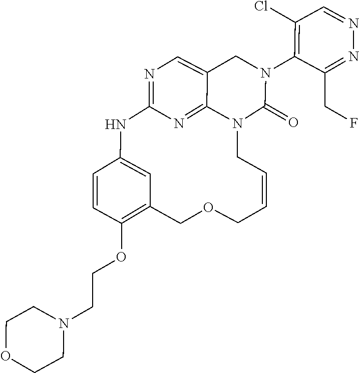 [SMILES: O=C1N2C/C=C\COCc3cc(ccc3OCCN3CCOCC3)Nc3ncc(c2n3)CN1c1c(Cl)cnnc1CF] | (Z)-1³-(5-chloro-3-(fluoromethyl)pyridazin-4-yl)-3⁴-(2- morpholinoethoxy)-1¹,1²,1³,1⁴-tetrahydro-5-oxa-2-aza- 1(7,1)-pyrimido[4,5-d]pyrimidina-3(1,3)- benzenacyclononaphan-7-en-1²-one |
| AC15 | 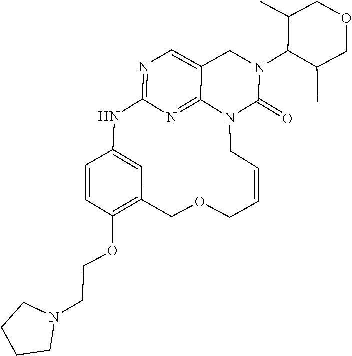 [SMILES: CC1COCC(C)C1N1Cc2cnc3nc2N(C/C=C\COCc2cc(ccc2OCCN2CCCC2)N3)C1=O] | (Z)-1³-(3,5-dimethyltetrahydro-2H-pyran-4-yl)-3⁴-(2- (pyrrolidin-1-yl)ethoxy)-1¹,1²,1³,1⁴-tetrahydro-5-oxa-2-aza- 1(7,1)-pyrimido[4,5-d]pyrimidina-3(1,3)- benzenacyclononaphan-7-en-1²-one |
| AC16 | 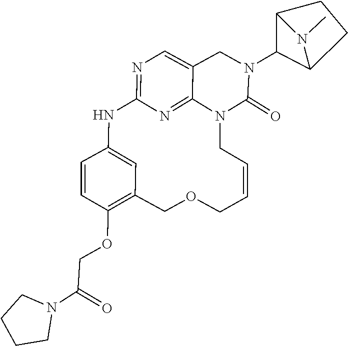 [SMILES: CN1C2CCC1C2N1Cc2cnc3nc2N(C/C=C\COCc2cc(ccc2OCC(=O)N2CCCC2)N3)C1=O] | (Z)-1³-(5-methyl-5-azabicyclo[2.1.1]hexan-6-yl)-3⁴-(2-oxo-2- (pyrrolidin-1-yl)ethoxy)-1¹,1²,1³,1⁴-tetrahydro-5-oxa-2-aza- 1(7,1)-pyrimido[4,5-d]pyrimidina-3(1,3)- benzenacyclononaphan-7-en-1²-one |
| AC17 | 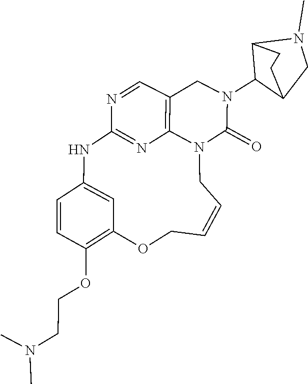 [SMILES: CN(C)CCOc1ccc2cc1OC/C=C\CN1C(=O)N(C3C4CCC3N(C)C4)Cc3cnc(nc31)N2] | (Z)-3⁴-(2-(dimethylamino)ethoxy)-1³-(2-methyl-2- azabicyclo[2.2.1]heptan-7-yl)-1¹,1²,1³,1⁴-tetrahydro-4-oxa-2- aza-1(7,1)-pyrimido[4,5-d]pyrimidina-3(1,3)- benzenacyclooctaphan-6-en-1²-one |
| AC18 | 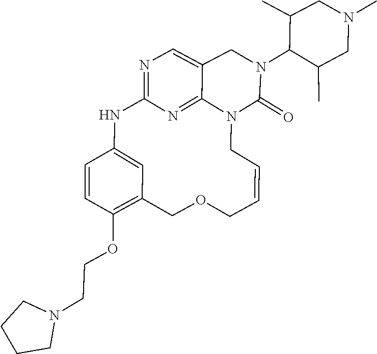 [SMILES: CC1CN(C)CC(C)C1N1Cc2cnc3nc2N(C/C=C\COCc2cc(ccc2OCCN2CCCC2)N3)C1=O] | (Z)-3⁴-(2-(pyrrolidin-1-yl)ethoxy)-1³-(1,3,5-trimethylpiperidin- 4-yl)-1¹,1²,1³,1⁴-tetrahydro-5-oxa-2-aza-1(7,1)-pyrimido[4,5- d]pyrimidina-3(1,3)-benzenacyclononaphan-7-en-1²-one |
| AC18 | 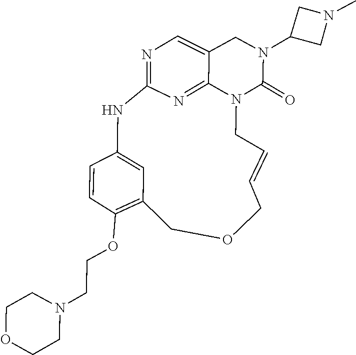 [SMILES: CN1CC(N2Cc3cnc4nc3N(C/C=C/COCc3cc(ccc3OCCN3CCOCC3)N4)C2=O)C1] | (E)-1³-(1-methylazetidin-3-yl)-3⁴-(2-morpholinoethoxy)- 1¹,1²,1³,1⁴-tetrahydro-5-oxa-2-aza-1(7,1)-pyrimido[4,5- d]pyrimidina-3(1,3)-benzenacyclononaphan-7-en-1²-one |

In particular embodiments, the compound of the invention is selected from the group consisting of AA1, AA2, AA3, AM4, AA5, AA6, AA7, AA8, AA9, AA10, AA11, AA12, AA13, AA14, AA15, AA16, AA17, AA18, AA19, AA20, AA21, AA22, AA23 and AA24; and also their solvates, salts, N-oxides, complexes, polymorphs, crystalline forms, racemic mixtures, diastereomers, enantiomers, tautomers, conformers, isotopically labelled forms, prodrugs, and combinations thereof.

In particular embodiments, the compound of the invention is selected from the group consisting of AA1, AA2, AA3, AA6, AA7, AA8, AA9, AA10, AA11, AA12, AA13, AA23 and AA24; and also their solvates, salts, N-oxides, complexes, polymorphs, crystalline forms, racemic mixtures, diastereomers, enantiomers, tautomers, conformers, isotopically labelled forms, prodrugs, and combinations thereof.

In particular embodiments, the compound of the invention is selected from the group consisting of AA4; and also their solvates, salts, N-oxides, complexes, polymorphs, crystalline forms, racemic mixtures, diastereomers, enantiomers, tautomers, conformers, isotopically labelled forms, prodrugs, and combinations thereof.

In particular embodiments, the compound of the invention is selected from the group consisting of AA5, AA18 and AA19; and also their solvates, salts, N-oxides, complexes, polymorphs, crystalline forms, racemic mixtures, diastereomers, enantiomers, tautomers, conformers, isotopically labelled forms, prodrugs, and combinations thereof.

In particular embodiments, the compound of the invention is selected from the group consisting of AA14, AA15, AA16 and AA17; and also their solvates, salts, N-oxides, complexes, polymorphs, crystalline forms, racemic mixtures, diastereomers, enantiomers, tautomers, conformers, isotopically labelled forms, prodrugs, and combinations thereof.

In particular embodiments, the compound of the invention is selected from the group consisting of AA20; and also their solvates, salts, N-oxides, complexes, polymorphs, crystalline forms, racemic mixtures, diastereomers, enantiomers, tautomers, conformers, isotopically labelled forms, prodrugs, and combinations thereof.

In particular embodiments, the compound of the invention is selected from the group consisting of AA21 and AA22; and also their solvates, salts, N-oxides, complexes, polymorphs, crystalline forms, racemic mixtures, diastereomers, enantiomers, tautomers, conformers, isotopically labelled forms, prodrugs, and combinations thereof.

In particular embodiments, the compound of the invention is selected from the group consisting of AA2, AA3, AA6, AA7, AA8, AA9, AA10, AA11, AA12, AA13, AA16, AA17, AA18, AA19, AA23 and AA24; and also their solvates, salts, N-oxides, complexes, polymorphs, crystalline forms, racemic mixtures, diastereomers, enantiomers, tautomers, conformers, isotopically labelled forms, prodrugs, and combinations thereof.

In particular embodiments, the compound of the invention is selected from the group consisting of AA2, AA3, AA6, AA7, AA8, AA9, AA10, AA11, AA12, AA13, AA23 and AA24; and also their solvates, salts, N-oxides, complexes, polymorphs, crystalline forms, racemic mixtures, diastereomers, enantiomers, tautomers, conformers, isotopically labelled forms, prodrugs, and combinations thereof.

In particular embodiments, the compound of the invention is selected from the group consisting of AA1, AA2, AA3, AA4, AA5, AA6, AA7, AA8, AA9, AA10, AA11, AA12 and AA13; and also their solvates, salts, N-oxides, complexes, polymorphs, crystalline forms, racemic mixtures, diastereomers, enantiomers, tautomers, conformers, isotopically labelled forms, prodrugs, and combinations thereof.

In particular embodiments, the compound of the invention is selected from the group consisting of AA2, AA3, AA6, AA7, AA8, AA9, AA10, AA11, AA12 and AA13; and also their solvates, salts, N-oxides, complexes, polymorphs, crystalline forms, racemic mixtures, diastereomers, enantiomers, tautomers, conformers, isotopically labelled forms, prodrugs, and combinations thereof.

In particular embodiments, the compound of the invention is selected from the group consisting of AC1, AC2, AC3, AC4, AC5, AC6, AC7, AC8, AC9, AC10, AC11, AC12, AC13, AC14, AC15, AC16, AC17 and AC18, and also their solvates, salts, N-oxides, complexes, polymorphs, crystalline forms, racemic mixtures, diastereomers, enantiomers, tautomers, conformers, isotopically labelled forms, prodrugs, and combinations thereof.

In particular embodiments, the compound of the invention is selected from the group consisting of AC1, AC2, AC3, AC4, AC5, AC6, AC7, AC8, AC9, AC10, AC11 and AC12, and also their solvates, salts, N-oxides, complexes, polymorphs, crystalline forms, racemic mixtures, diastereomers, enantiomers, tautomers, conformers, isotopically labelled forms, prodrugs, and combinations thereof.

In one certain embodiment, the compound of the invention is AA3 or AA6, or a solvate, salt, N-oxide, complex, polymorph, crystalline form, tautomer, conformer, isotopically labelled form, prodrug, or combination thereof.

In one certain embodiment, the compound of the invention is AA11 or AA12, or a solvate, salt, N-oxide, complex, polymorph, crystalline form, tautomer, conformer, isotopically labelled form, prodrug, or combination thereof.

In one certain embodiment, the compound of the invention is AA3 or AA5, or a solvate, salt, N-oxide, complex, polymorph, crystalline form, tautomer, conformer, isotopically labelled form, prodrug, or combination thereof.

In one certain embodiment, the compound of the invention is AA7, or a solvate, salt, N-oxide, complex, polymorph, crystalline form, tautomer, conformer, isotopically labelled form, prodrug, or combination thereof.

In one certain embodiment, the compound of the invention is AA1, or a solvate, salt, N-oxide, complex, polymorph, crystalline form, tautomer, conformer, isotopically labelled form, prodrug, or combination thereof.

In certain aspects herein, a compound used in the invention may be, for example, ARN-3261 (Vankayalapati et al 2017, AACR Cancer Res 77(13 Suppl):Abstract nr LB-296; U.S. Pat. Nos. 9,260,426, 9,890,153, 9,951,062).

In certain embodiments, the invention may relate to a solvate, salt, N-oxide, complex, racemic mixture, diastereomer, enantiomer, tautomer, conformer, isotopically labeled form, prodrug, or combination thereof, of any of the compounds of the invention or of any of the compounds used in the invention; such as a solvate, salt, complex, racemic mixture, diastereomer, enantiomer, tautomer, conformer, isotopically labeled form, or combination thereof, or such compound; such as a solvate, salt, racemic mixture, diastereomer, enantiomer, tautomer, conformer, isotopically labeled form, or combination thereof, or such compound.

In one embodiment the compounds of the invention (or the compounds used in the invention) do not encompass compounds of one or more of the following groups (1) to (4) of formula (I) or (VIIa) (in the groups (1) to (4) of a moiety (such as a methyl) is unsubstituted unless it is explicitly specified that such moiety is substituted):

(1) (w) pyrimido[4,5-d]pyrimidin-2(1H)-one, 1-[3-[2-[[(1,1-dimethylethyl)diphenylsilyl]oxy]ethyl]phenyl]-3-(2-furanylmethyl)-3,4-dihydro-7-(phenylamino)—[CAS: 266315-42-8]; (x) pyrimido[4,5-d]pyrimidin-2(1H)-one, 1-[3-[2-[[(1,1-dimethylethyl)diphenylsilyl]oxy]ethyl]phenyl]-3,4-dihydro-3-(1-oxido-3-pyridinyl)-7-(phenylamino)—[CAS: 266315-40-6]; (y) pyrimido[4,5-d]pyrimidin-2(1H)-one, 3-(2-furanylmethyl)-3,4-dihydro-1-[3-(2-hydroxyethyl)phenyl]-7-(phenylamino)—[CAS: 266313-80-8]; and/or (z) pyrimido[4,5-d]pyrimidin-2(1H)-one, 3,4-dihydro-1-[3-(2-hydroxyethyl)phenyl]-3-(1-oxido-3-pyridinyl)-7-(phenylamino)—[CAS: 266313-79-5];
(2) when both R^{4a} and R^{4b} are H, then R⁵ consists of: (x) 2-furanylmethyl; or (y) 1-oxido-3-pyridinyl;
(3) when both R^{4a} and R^{4b} are H, then R⁵ consists of thienyl, furyl, pyridyl, pyrimidinyl, quinolyl, indolyl, benzofuranyl, imidazole, 1,2,3-triazole, 1,2,4-triazole, tetrazole, thiazole, and/or pyridine-N-oxide, in each case optionally mono- or multiply-substituted by halogen, lower alkyl, lower alkoxy, lower-alkoxy lower alkyl, trifluoromethyl, hydroxy, hydroxy lower-alkyl, carboxylic acid, carboxylic ester, nitro, amino or phenyl, and/or by a group of the formula —Z^{x}—NR^{4x}R^{5x} or —Z^{x}—OR^{6x} in which Z^{x} represents a spacer group and R^{4x} and R^{5x} each individually represent hydrogen or lower alkyl or R^{4x} and R^{5x} together with the nitrogen atom to which they are attached represent a 4-, 5- or 6-membered saturated or partially unsaturated or 5- or 6-membered aromatic heterocyclic group which contains one or more hetero atoms selected from nitrogen, sulphur and oxygen and which is optionally substituted by lower alkyl, lower alkoxy and/or oxo and/or which is optionally benz-fused, and in which R^{6x} is defined as H or lower-alkyl, preferably H. Examples of spacer groups are —(CH)ₘₓ— in which mx stands for 1, 2, 3 or 4 and —O(CH₂)ₙₓ— in which nx stands for 2, 3 or 4; and/or
(4) when both R^{4a} and R^{4b} are H, and R⁶ comprises either pyridl or thienyl, then neither R¹ nor R³ is or is substituted with the groups —NH—(C═O)—CH═CH—CH₂—N(CH₃)₂, —NH—(C═O)—CH═CH₂ and/or —NH—(C═O)—CH₂—CH₃.

The compounds of the invention (and/or the compounds used in the invention) which contain a basic functionality may form salts with a variety of inorganic or organic acids. The compounds of the invention (and/or the compounds used in the invention) which contain an acidic functionality may form salts with a variety of inorganic or organic bases. Exemplary inorganic and organic acids/bases as well as exemplary acid/base addition salts of the compounds of the present invention (or of the compounds used in the invention) are given in the definition of “pharmaceutically acceptable salt” in the section “Pharmaceutical composition”, below. The compounds of the invention (and/or the compounds used in the invention) which contain both basic and acidic functionalities may be converted into either base or acid addition salt. The neutral forms of the compounds of the invention (or of the compounds used in the invention) may be regenerated by contacting the salt with a base or acid and isolating the parent compound in the conventional manner.

The compounds of the invention (and/or the compounds used in the invention) may be in the form of an N-oxide, i.e., they can contain the functional group functional group ≡N⁺—O⁻ (e.g., (Rⁿ)₃N⁺—O⁻, i.e., an N—O coordinate covalent bond, wherein Rⁿ is independently selected from the group consisting of hydrogen, alkyl, alkenyl, alkynyl, aryl, heteroaryl, cycloalkyl, and heterocyclyl, wherein each of the alkyl, alkenyl, alkynyl, aryl, heteroaryl, cycloalkyl, and heterocyclyl groups is optionally substituted with one or more (such as 1 to the maximum number of hydrogen atoms bound to the alkyl, alkenyl, alkynyl, aryl, heteroaryl, cycloalkyl, or heterocyclyl group, e.g., 1, 2, 3, 4, 5, 6, 7, 8, 9, or up to 10, such as between 1 to 5, 1 to 4, or 1 to 3, or 1 or 2) independently selected R³⁰, the R³⁰ preferably being a 1^{st} level substituent, a 2^{nd} level substituent, or a 3^{rd} level substituent as specified herein). Particular examples of N-oxides of compounds of the invention (or of the compounds used in the invention) are those, wherein R^{1a} and/or R⁶) contains the functional group —N⁺—O⁻. Non-limiting examples of R^{1a} substituents which can occur as N-oxides include the following, and also include (to the extent applicable in respect of the position of the indicated N-oxide) the piperidinyl substituents corresponding thereto:

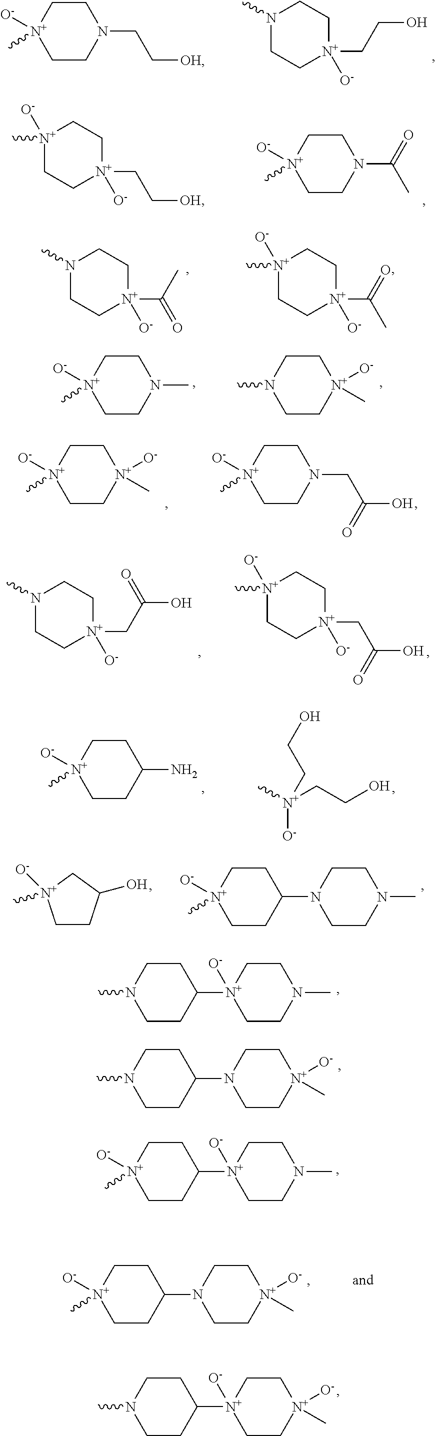

wherein [symbol] represents the bond by which the R⁶ substituent is bound to the remainder of the compound.

Non-limiting examples of R⁶ substituents which can occur as N-oxides include the following:

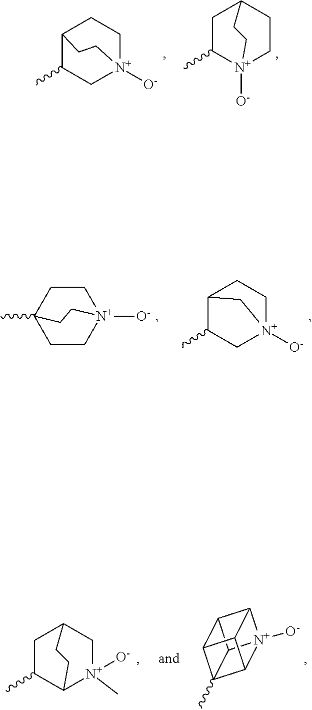

wherein [symbol] represents the bond by which the R⁶ substituent is bound to the remainder of the compound.

The come invention (and/or the compounds used in the invention) may be in a prodrug form. Prodrugs of the compounds of the invention (or of the compounds used in the invention) are those compounds that upon administration to an individual undergo chemical conversion under physiological conditions to provide the compounds of the invention. Additionally, prodrugs can be converted to the compounds of the invention (or of the compounds used in the invention) by chemical or biochemical methods in an ex vivo environment. For example, prodrugs can be slowly converted to the compounds of the invention (or to the compounds used in the invention) when, for example, placed in a transdermal patch reservoir with a suitable enzyme or chemical reagent. Exemplary prodrugs are esters (using an alcohol or a carboxy group contained in the kinase inhibitor of the invention (or in the compounds used in the invention)) or amides (using an amino or a carboxy group contained in the kinase inhibitor of the invention (or in the compounds used in the invention)) which are hydrolyzable in vivo. Specifically, any amino group which is contained in the kinase inhibitor of the invention (or in the compounds used in the invention) and which bears at least one hydrogen atom can be converted into a prodrug form. Typical N-prodrug forms include carbamates (1), Mannich bases (2), enamines (3), and enaminones (4).

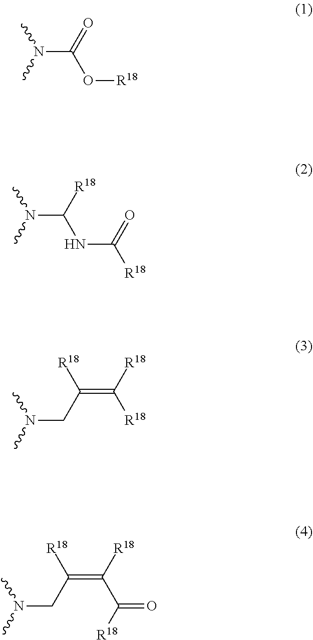

wherein R¹⁸ is independently selected from the group consisting of H, alkyl, alkenyl, alkynyl, cycloalkyl, aryl, heteroaryl, and heterocyclyl, wherein each of the alkyl, alkenyl, alkynyl, cycloalkyl, aryl, heteroaryl, and heterocyclyl groups is optionally substituted with one or more (such as 1 to the maximum number of hydrogen atoms bound to the alkyl, alkenyl, alkynyl, cycloalkyl, aryl, heteroaryl, or heterocyclyl group, e.g., 1, 2, 3, 4, 5, 6, 7, 8, 9, or up to 10, such as between 1 to 5, 1 to 4, or 1 to 3, or 1 or 2) independently selected R³⁰, wherein R³⁰ is as defined herein (preferably, each R³⁰ is independently a 1^{st} level substituent, a 2^{nd} level substituent, or a 3^{rd} level substituent as specified herein). The prodrug properties (such as solubility, permeability, stability, how fast cleaved, where in the body cleaved under what conditions, target specificity, etc.) can be fine-tuned via modification of R¹⁸.

In one embodiment, a compound of the invention (and/or a compound used in the invention) is in the form of an N-oxide of a N atom comprised in R^{1a}.

For those compounds of the invention having any one of formulas (I), (Ia), (II), (III), (IV), (V), (Va), (VI), (VII), (VIIa), (VIII), (IX), (X), (XI), and (XII) (or for those compounds use in the invention)) and bearing one or more hydroxyl (i.e., —OH) groups, a further particular prodrug form is that wherein at least one of these two or more hydroxyl groups is derivatized to be a moiety selected from the group consisting of —OP(O)(OR^{11a})₂, —O(CH₂)₁₋₃—R¹⁹, —OC(═X^{a})R^{11a}, and —OC(═X^{a})X^{a}R^{11a}, wherein R^{11a} is independently selected from the group consisting of H, alkyl, alkenyl, alkynyl, cycloalkyl, aryl, heteroaryl, and heterocyclyl, wherein each of the alkyl, alkenyl, alkynyl, cycloalkyl, aryl, heteroaryl, and heterocyclyl groups is optionally substituted with one or more (such as 1 to the maximum number of hydrogen atoms bound to the alkyl, alkenyl, alkynyl, cycloalkyl, aryl, heteroaryl, or heterocyclyl group, e.g., 1, 2, 3, 4, 5, 6, 7, 8, 9, or up to 10, such as between 1 to 5, 1 to 4, or 1 to 3, or 1 or 2) independently selected R³⁰; R¹⁹ is independently selected from the group consisting of —OP(O)(OR^{11a})₂, —X^{a}C(═X^{a})R^{11a}, —X^{a}C(═X^{a})X^{a}R^{11a}, and 5-alkyl-2-oxo-1,3-dioxolo-4-yl; X^{a} is independently selected from O, S, and NH; and the —(CH₂)₁₋₃— group is optionally substituted with one or two independently selected R³⁰, wherein R³⁰ is as defined herein (preferably, each R³⁰ is independently selected from the group consisting of a 1^{st} level substituent, a 2^{nd} level substituent, and a 3^{rd} level substituent as specified herein). In one embodiment of this prodrug form of the compounds of the invention (or of the compounds used in the invention), the at least one derivatized hydroxyl group is selected from the group consisting of —OP(O)(OR^{11a})₂, —O(CH₂)₁₋₃—R¹⁹, —OC(═O)R^{11a}, and —OC(O)OR^{11a}, wherein R^{11a} is independently selected from the group consisting of H and C₁₋₆ alkyl (preferably C₁₋₃ alkyl), wherein the alkyl group is optionally substituted with one or two substituents independently selected from halogen, —OH, —OCH₃, —SCH₃, 2-(N,N-dimethylamino)ethoxy, and —NH_{2-z}(CH₃)_{z}, wherein z is 0, 1, or 2; R¹⁹ is independently selected from the group consisting of —OP(O)(OR^{11a})₂, —OC(═O)R^{11a}, —OC(═O)OR^{11a}, and 5-(C₁₋₃ alkyl)-2-oxo-1,3-dioxolo-4-yl; and the —(CH₂)₁₋₃— group is optionally substituted with one or two substituents independently selected from the group consisting of halogen, —OH, —OCH₃, —SCH₃, 2—(N,N-dimethylamino)ethoxy, and —NH_{2-z}(CH₃)_{z}, wherein z is 0, 1, or 2.

In certain embodiments, the invention (or a compound used in the invention) may relate to a solvate, salt, N-oxide, complex, racemic mixture, diastereomer, enantiomer, tautomer, conformer, isotopically labeled form, or combination thereof, of any of such prodrug; such as a solvate, salts, complex, racemic mixture, diastereomer, enantiomer, tautomer, conformer, isotopically labeled form, or combination thereof, of such prodrug.

In a one particular embodiment, a compound of the invention (or a compound used in the invention) is a solvate of a kinase inhibitor as specified under the heading “Compounds” (e.g., a kinase inhibitor having the general formula (I), (Ia), (II), (III), (IV), (Va), (Va), (VI), (VII), (VIIa), (VIII), (IX), (X), (XI) or (XII), or salt (in particular a pharmaceutically acceptable salt), N-oxide (in particular, N-oxides of R^{1a} and/or R⁶), complex, polymorph, crystalline form, racemic mixture, diastereomer, enantiomer, tautomer, conformer, isotopically labeled form, prodrug and/or having at least one derivatized hydroxyl group, as specified above, or a solvate, salt, N-oxide, complex, polymorph, crystalline form, racemic mixture, diastereomer, enantiomer, tautomer, conformer, isotopically labeled form or combination thereof), or combination thereof).

A compound of the invention (e.g., as specified under the heading “Compounds”) can, in certain embodiments, be in (e.g., provided in) a purified or (e.g., substantially) pure form. For example, the compound may be greater than about 50% pure, such as greater than about 60%, 70% or 80% pure, suitably greater than about 90% pure (in particular, greater than about 95%, 97% 98% and even 99%). That is, in certain of such embodiments such a compound is present together with only a limited amount of impurities (e.g., such as those introduced during manufacturing), such as only small amounts of impurities are present, including embodiments where the compound is present in a form where impurities are substantially absent. The purity (e.g., the absence, or degree of presence of impurities) of the compound can be determined by routine procedures e.g. by HLPC.

In one embodiment, the present invention provides such a compound containing less than about 50%, 40%, 30% and suitably 10% or 5% area by HPLC, preferably less than about 3% and 2% area by HPLC, more preferably less than 1% area by HPLC, of total impurities. The term “% area by HPLC” as used herein refers to the area in an HPLC chromatogram of one or more peaks compared to the total area of all peaks in the HPLC chromatogram expressed in percent of the total area. Further, the purity of the compound may be expressed herein as “HPLC” purity. As such, “HPLC purity”, is a calculation of the area under the compound peak divided by the total area under the curve in an HPLC chromatogram. Suitably, the compound contains less than about 10% area by HPLC of total impurities. More preferably, less than about 5% area by HPLC of total impurities.

In a further aspect, the present invention provides a compound of the invention (in particular those specified above with respect to any of formulas (I), (Ia), (II), (III), (IV), (V), (Va), (VI), (VII), (VIIa), (VIII), (IX), (X), (XI) or (XII)) for use as medicament, for example for use in therapy.

As it is evident from the examples, the inventors have found that the compounds of the invention as well as other structurally similar compounds inhibit one or more protein-tyrosine kinases, including any of those selected from the group consisting of: SIK3, SIK2, SIK1, ABL/BCR-ABL, KIT, NEK2, BRAF. CSF1R, HCK, TEC-family kinases (eg BTK, TXK or ITK), AXL, BLK, TYRO3, MERTK, ZAP70, SYK, EGFR and BRK; and/or selected from the group consisting of: FLT3, LCK, PHA2, EPHA4, ACK1, NEK11, WEE1, WNK2, Aurora-A, Aurora-B and TBK1.

In one embodiment, the compounds of the invention exhibit a different inhibition profile of kinases to the kinases inhibited by YKL-05-099 (compound PY1 herein), in particular. In one embodiment, the compounds of the invention are kinase inhibitors which: (i) are more potent at inhibiting or more specific to one or more key disease-related kinases (e.g., SIK3, SIK2, SIK1, ABL/BCR-ABL, KIT, NEK2, BRAF. CSF1R, HCK, TEC-family kinases (eg BTK, TXK or ITK), AXL, BLK, TYRO3, MERTK, ZAP70, SYK, EGFR, and/or BRK), especially to SIK3, relative to other kinases, than the specificity shown by YKL-05-099 to one or more such other kinases (eg, compounds AA3, and/or AA6); (ii) inhibit key disease- (eg SIK3), side-effect-related or other kinases in a different inhibition profile than YKL-05-099 (e.g. to SIK2, SIK1, KIT. LCK and/or FLT3) (eg, compounds AA11, AA5 and/or AA12); (iii) exhibit a (eg substantial) different inhibition profile of kinase inhibition over a plurality of (eg, 100 or more) of different kinases (eg, compounds AA3 and/or AA5); and/or (iv) inhibit one or more mutant of a disease-related kinase, in particular a mutant that is resistant to one or other kinase inhibitor, such as mutants of ABL/BCR-ABL or KIT.

In another embodiment, the compounds of the invention exhibit properties against in-vitro cultured cell lines that are different to (eg, superior to) those of YKL-05-099. For example, in certain of such embodiments, compounds of the invention sensitise cells to the cell killing by TNF more potently than YKL-05-099 (eg, compound AA7).

In yet another embodiment, the compounds of the invention exhibit one or more pharmacological properties (eg, pharmacokinetics, and/or selectivity, bioavailability, toxicity, side effects, dosing, patient compliance, compatibility, stability, half-life, etc) that are different to those of YKL-05-099 (eg, compound AA1), and which may be—in certain embodiments—superior in at least one aspect to the pharmacological properties exhibited by YKL-05-099. Such differences in pharmacological properties can lead to the administration of compounds of the invention in different therapeutic regimens than eg YKL-05-099. Such properties may be one or more improved drug metabolism and pharmacokinetic (DMPK) properties such as those described in Example 8 (such as AUC, plasma concentration and/or free plasma concentration).

### Pharmaceutical Compositions

The compounds described in the present invention (in particular those specified above such as those of formula (I), (Ia), (II), (III), (IV), (V), (Va), (VI), (VII), (VIIa), (VIII), (IX), (X), (XI) or (XII) particularly those given in Table A and Table B) or the compounds used in the present invention are preferably administered to a patient in need thereof via a pharmaceutical composition. Thus, in a second aspect, the present invention provides a pharmaceutical composition comprising a compound (eg, a kinase inhibitor) as specified above under the heading “Compounds” (e.g., a kinase inhibitor having the general formula (I), (Ia), (II), (III), (IV), (V), (Va), (VI), (VII), (VIIa), (VIII), (IX), (X), (XI) or (XII), or a solvate, salt (in particular a pharmaceutically acceptable salt), N-oxide (in particular, an N-oxide of R^{1a} and/or R⁶), complex, polymorph, crystalline form, racemic mixture, diastereomer, enantiomer, tautomer, conformer, isotopically labeled form, prodrug and/or having at least one derivatized hydroxyl group, as specified above, or a solvate, salt, N-oxide, complex, polymorph, crystalline form, racemic mixture, diastereomer, enantiomer, tautomer, conformer, isotopically labeled form or combination thereof), or combination thereof) and optionally one or more pharmaceutically acceptable excipients.

Thus, in one embodiment the pharmaceutical composition comprises a kinase inhibitor as specified above under the heading “Compounds” (in particular a compound of the first aspect of the invention) and one or more pharmaceutically acceptable excipients. Furthermore, the pharmaceutical composition may further comprise one or more additional therapeutic agents. Thus, in particular embodiments, the pharmaceutical composition comprises (i) a kinase inhibitor as specified above under the heading “Compounds” (in particular a compound of the first aspect of the invention) and one or more additional therapeutic agents; or (ii) a kinase inhibitor as specified above under the heading “Compounds” (in particular a compound of the first aspect of the invention), one or more additional therapeutic agents, and one or more pharmaceutically acceptable excipients.

The term “pharmaceutically acceptable” refers to the non-toxicity of a material which does not interact with the (e.g., therapeutic) action of the active component (e.g., a kinase inhibitor of the invention (or a compound used in the invention), either alone or in combination with one or more additional therapeutic agents) of the pharmaceutical composition.

The pharmaceutical composition may be administered to an individual by any route, such as enterally or parenterally.

The expressions “enteral administration” and “administered enterally” as used herein mean that the drug administered is taken up by the stomach and/or the intestine. Examples of enteral administration include oral and rectal administration. The expressions “parenteral administration” and “administered parenterally” as used herein mean modes of administration other than enteral administration, usually by injection or topical application, and include, without limitation, intravenous, intramuscular, intraarterial, intrathecal, intracapsular, intraosseous, intraorbital, intracardiac, intradermal, intraperitoneal, transtracheal, subcutaneous, subcuticular, intraarticular, subcapsular, intracerebral, intracerebroventricular, subarachnoid, intraspinal, epidural and intrasternal administration (such as by injection and/or infusion) as well as topical administration (e.g., epicutaneous, inhalational, or through mucous membranes (such as buccal, sublingual or vaginal)).

Dosage forms for topical and/or transdermal administration of a compound described herein may include ointments, pastes, creams, lotions, gels, powders, solutions, sprays, inhalants, and/or patches. Generally, the active ingredient is admixed under sterile conditions with a pharmaceutically acceptable excipient such as one or more pharmaceutical carriers) and/or any needed preservatives and/or buffers as can be required. Additionally, the present disclosure contemplates the use of transdermal patches, which often have the added advantage of providing controlled delivery of an active ingredient to the body. Such dosage forms can be prepared, for example, by dissolving and/or dispensing the active ingredient in the proper medium. Alternatively, or additionally, the rate can be controlled by either providing a rate controlling membrane and/or by dispersing the active ingredient in a polymer matrix and/or gel.

Suitable devices for use in delivering intradermal pharmaceutical compositions described herein include short needle devices. Intradermal compositions can be administered by devices which limit the effective penetration length of a needle into the skin. Alternatively, or additionally, conventional syringes can be used in the classical mantoux method of intradermal administration. Jet injection devices which deliver liquid formulations to the dermis via a liquid jet injector and/or via a needle which pierces the stratum corneum and produces a jet which reaches the dermis are suitable. Ballistic powder/particle delivery devices which use compressed gas to accelerate the compound in powder form through the outer layers of the skin to the dermis are suitable.

Formulations suitable for topical administration include, but are not limited to, liquid and/or semi-liquid preparations such as liniments, lotions, oil-in-water and/or water-in-oil emulsions such as creams, ointments, and/or pastes, and/or solutions and/or suspensions. Topically administrable formulations may, for example, comprise from about 0.1% to about 10% (w/w) active ingredient, although the concentration of the active ingredient can be as high as the solubility limit of the active ingredient in the solvent. Formulations for topical administration may further comprise one or more of the additional ingredients described herein.

The compounds of the present invention (or the compounds used in the present invention) are generally applied in “pharmaceutically acceptable amounts” and in “pharmaceutically acceptable preparations”. Such compositions may contain salts, buffers, preserving agents, carriers and optionally other therapeutic agents. “Pharmaceutically acceptable salts” comprise, for example, acid addition salts which may, for example, be formed by mixing a solution of compounds with a solution of a pharmaceutically acceptable acid such as hydrochloric acid, sulfuric acid, fumaric acid, maleic acid, succinic acid, acetic acid, benzoic acid, citric acid, tartaric acid, carbonic acid or phosphoric acid. Furthermore, where the compound carries an acidic moiety, suitable pharmaceutically acceptable salts thereof may include alkali metal salts (e.g., sodium or potassium salts); alkaline earth metal salts (e.g., calcium or magnesium salts); and salts formed with suitable organic ligands (e.g., ammonium, quaternary ammonium and amine cations formed using counteranions such as halide, hydroxide, carboxylate, sulfate, phosphate, nitrate, alkyl sulfonate and aryl sulfonate). Illustrative examples of pharmaceutically acceptable salts include, but are not limited to, acetate, adipate, alginate, arginate, ascorbate, aspartate, benzenesulfonate, benzoate, bicarbonate, bisulfate, bitartrate, borate, bromide, butyrate, calcium edetate, camphorate, camphorsulfonate, camsylate, carbonate, chloride, citrate, clavulanate, cyclopentanepropionate, digluconate, dihydrochloride, dodecylsulfate, edetate, edisylate, estolate, esylate, ethanesulfonate, formate, fumarate, galactate, galacturonate, gluceptate, glucoheptonate, gluconate, glutamate, glycerophosphate, glycolylarsanilate, hemisulfate, heptanoate, hexanoate, hexylresorcinate, hydrabamine, hydrobromide, hydrochloride, hydroiodide, 2-hydroxy-ethanesulfonate, hydroxynaphthoate, iodide, isobutyrate, isothionate, lactate, lactobionate, laurate, lauryl sulfate, malate, maleate, malonate, mandelate, mesylate, methanesulfonate, methylsulfate, mucate, 2-naphthalenesulfonate, napsylate, nicotinate, nitrate, N-methylglucamine ammonium salt, oleate, oxalate, pamoate (embonate), palmitate, pantothenate, pectinate, persulfate, 3-phenylpropionate, phosphate/diphosphate, phthalate, picrate, pivalate, polygalacturonate, propionate, salicylate, stearate, sulfate, suberate, succinate, tannate, tartrate, teoclate, tosylate, triethiodide, undecanoate, valerate, and the like (see, for example, Berge et al., “Pharmaceutical Salts”, J. Pharm. Sci., 66, pp. 1-19 (1977)).

The term “excipient” when used herein is intended to indicate all substances in a pharmaceutical composition which are not active ingredients (e.g., which are therapeutically inactive ingredients that do not exhibit any therapeutic effect in the amount/concentration used), such as, e.g., carriers, binders, lubricants, thickeners, surface active agents, preservatives, stabilizers, emulsifiers, buffers, flavoring agents, colorants, or antioxidants.

The compositions described in the present invention may comprise a pharmaceutically acceptable carrier. As used herein, “pharmaceutically acceptable carrier” includes any and all solvents, dispersion media, coatings, isotonic and absorption delaying agents, and the like that are physiologically compatible. The “pharmaceutically acceptable carrier” may be in the form of a solid, semisolid, liquid, or combinations thereof. Preferably, the carrier is suitable for enteral (such as oral) or parenteral administration (such as intravenous, intramuscular, subcutaneous, spinal or epidermal administration (e.g., by injection or infusion)). Depending on the route of administration, the active compound, e.g., the compound of the present invention (or the compound used in the present invention), either alone or in combination with one or more additional therapeutic agents, may be coated in a material to protect the active compound(s) from the action of acids and other natural conditions that may inactivate the active compound.

Examples of suitable aqueous and non-aqueous carriers which may be employed in the pharmaceutical compositions according to the present invention include water (e.g., water for injection), ethanol, polyols (such as glycerol, propylene glycol, polyethylene glycol, and the like), aqueous solutions of a salt, carbohydrate, sugar alcohol, or an amino acid (such as saline or an aqueous amino acid solution), and suitable mixtures and/or buffered forms thereof, vegetable oils (such as olive oil), and injectable organic esters (such as ethyl oleate). Proper fluidity can be maintained, for example, by the use of coating materials, such as lecithin, by the maintenance of the required particle size in the case of dispersions, and by the use of surfactants.

Pharmaceutically acceptable carriers include sterile aqueous solutions or dispersions and sterile powders for the extemporaneous preparation of sterile injectable solutions or dispersions. The use of such media and agents for pharmaceutically active compounds is known in the art. Except insofar as any conventional media or agent is incompatible with the active compound, use thereof in the pharmaceutical compositions according to the present invention is contemplated.

Additional therapeutic agents can be administered together with, before or after the compound of the present invention or with, before or after the compound used in the invention (in particular that specified above such as those of formula (I), (Ia), (II), (III), (IV), (V), (Va), (VI), (VII), (VIIa), (VIII), (IX), (X), (XI) or (XII)) or incorporated into the compositions. In one embodiment, the pharmaceutical composition described herein comprises a kinase inhibitor of the invention as described above (or a compound as used in the present invention) (e.g. having the general formula (I), (Ia), (II), (III), (IV), (V), (Va), (VI), (VII), (VIIa), (VIII), (IX), (X), (XI) or (XII) or a solvate, salt (in particular a pharmaceutically acceptable salt), N-oxide (in particular, an N-oxide of R^{1a} or R⁶), complex, polymorph, crystalline form, racemic mixture, diastereomer, enantiomer, tautomer, conformer, isotopically labeled form, prodrug and/or having at least one derivatized hydroxyl group, as specified above, or a solvate, salt, N-oxide, complex, polymorph, crystalline form, racemic mixture, diastereomer, enantiomer, tautomer, conformer, isotopically labeled form or combination thereof), or combination of any of the foregoing), at least one additional therapeutic agent, and one or more pharmaceutically acceptable excipients.

The “additional therapeutic agent” (which in one embodiment is not a kinase inhibitor of formula (I), as specified herein, or in another embodiment (i) is not a kinase inhibitor of formula (I), as specified herein; and (ii) is not a compound of formula (VIIa),, or in another embodiment may be a different kinase inhibitor of formula (I), or in another embodiment may be a different kinase inhibitor of formula or (VIIa))) may be selected from any compound which can be used in the treatment of a disorder, disease or condition being a proliferative disorder (e.g., a cancer, such as one described, defined or disclosed elsewhere herein), and/or caused by or associated with: (i) the (e.g., pathological or erroneous) expression and/or activity of kinase, such as SIK3, SIK2, SIK1, ABL/BCR-ABL, KIT, NEK2, BRAF. CSF1R, HCK, TEC-family kinases (eg BTK, TXK or ITK), AXL, BLK, TYRO3, MERTK, ZAP70, SYK, EGFR, and/or BRK and/or FLT3, LCK, PHA2, EPHA4, ACK1, NEK1l, WEE1, WNK2, Aurora-A, Aurora-B and TBK1 and/or (ii) cellular resistance to an (eg a cell-mediated) immune response. Examples of suitable additional therapeutic agents are defined or disclosed elsewhere herein, and include an immune checkpoint inhibitor (such as an inhibitor of PD1, PDL1, CTLA-4, LAG3 or IDO1, and in particular an immune checkpoint inhibitor selected from the list consisting of: nivolumab, relatlimab, ipilimumab and BMS-986205), TNF or an agonist of TNFR1- or TNFR2-signalling, adoptive cellular therapy including CAR T cells directed against a tumor antigen, vaccines including dendritic cell- (DC) based vaccination, or an agent that is capable of inducing or induces the exposure of the cells involved with the proliferative disorder to TNF or an agonist of TNFR1- and/or TNFR2-signalling, is administered to the subject. The additional therapeutic agent may induce an additive or synergistic therapeutic effect when used in combination with the compound of the invention.

The pharmaceutical composition described herein may comprise, in addition to the kinase inhibitor of the invention (and/or the compound used in the invention), at least one, e.g., 1, 2, 3, 4, 5, 6, 7 or 8, additional therapeutic agents. According to the present teaching, the at least one additional therapeutic agent may be formulated together with the kinase inhibitor of the invention (and/or with the compound used in the invention) in a single pharmaceutical composition. Alternatively, the pharmaceutical composition may be structured as kit of parts, wherein the kinase inhibitor of the invention (or the compound used in the invention) is provided in a first formulation and the at least one additional therapeutic agent is provided in a second formulation, i.e., a second pharmaceutical composition. The first and the second pharmaceutical compositions may be combined prior to use. In other words, before administering the pharmaceutical composition, a formulation comprising the additional therapeutic agent may be added to the first pharmaceutical composition comprising the kinase inhibitor of the invention (or the compound used in the invention). Alternatively, the present teaching envisages administering the kinase inhibitor of the invention (or the compound used in the invention) formulated in a first pharmaceutical composition and administering the at least one additional therapeutic agent formulated in a second pharmaceutical composition. The pharmaceutical compositions may be administered concomitantly or in succession. For example, the first pharmaceutical composition may be administered at a first point in time and the second pharmaceutical composition may be administered at a second point in time, wherein the points in time may be separated by, for example, 0, or up to 1, 2, 3, 4, 5 or 10 min, up to 1, 2, 3, 4, 5 or 10 hours, up to 1, 2, 3, 4, 5 or 10 days, up to 1, 2, 3, 4, 5 or 10 weeks, up to 1, 2, 3, 4, 5 or 10 months or up to 1, 2, 3, 4, 5 or 10 years.

The compositions may also contain adjuvants such as preservatives, stabilizers, wetting agents, emulsifying agents, pH buffering agents, and dispersing agents. Prevention of the presence of microorganisms may be ensured by sterilization procedures and/or by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

Regardless of the route of administration selected, the active compounds, which may be used in a suitable solvated form, and/or the pharmaceutical compositions according to the present invention, are formulated into pharmaceutically acceptable dosage forms by conventional methods known to those of skill in the art (cf., e.g., Remington, “The Science and Practice of Pharmacy” edited by Allen, Loyd V., Jr., 22nd edition, Pharmaceutical Sciences, September 2012; Ansel et al., “Pharmaceutical Dosage Forms and Drug Delivery Systems”, 7th edition, Lippincott Williams & Wilkins Publishers, 1999).

A pharmaceutical composition can be administered by a variety of methods known in the art. As will be appreciated by the skilled artisan, the route and/or mode of administration will vary depending upon the desired results. The pharmaceutical compositions containing one or more active compounds can be prepared with carriers that will protect the one or more active compounds against rapid release, such as a controlled release formulation, including implants, transdermal patches, and microencapsulated delivery systems. Biodegradable, biocompatible polymers can be used, such as ethylene vinyl acetate, polyanhydrides, polyglycolic acid, collagen, polyorthoesters, and polylactic acid. Methods for the preparation of such compositions are generally known to those skilled in the art. See, e.g., Sustained and Controlled Release Drug Delivery Systems, J. R. Robinson, ed., Marcel Dekker, Inc., New York, 1978.

To administer a compound of the present invention (or a compound used in the present invention) by certain routes of administration, it may be necessary to coat the compound with, or co-administer the compound with, a material to prevent its inactivation. For example, the compound may be administered to an individual in an appropriate carrier, for example, liposomes, or a diluent. Pharmaceutically acceptable diluents include saline and aqueous buffer solutions. Liposomes include water-in-oil-in-water CGF emulsions as well as conventional liposomes (Strejan et al., J. Neuroimmunol. 7: 27(1984)).

Pharmaceutical compositions typically are sterile and stable under the conditions of manufacture and storage. The composition can be formulated as a solution, microemulsion, liposome, or other ordered structure suitable to high drug concentration. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. In many cases, it will be preferable to include isotonic agents, for example, sugars, polyalcohols such as mannitol, sorbitol, or sodium chloride in the composition. Prolonged absorption of the injectable compositions can be brought about by including in the composition an agent that delays absorption, for example, monostearate salts and gelatin.

An injectable composition should be sterile and fluid to the extent that the composition is deliverable by syringe. In addition to water, the carrier can be an isotonic buffered saline solution, ethanol, polyol (for example, glycerol, propylene glycol, and liquid polyethylene glycol, and the like), and suitable mixtures thereof. Sterile injectable solutions can be prepared by incorporating the active compound in the required amount in an appropriate solvent with one or a combination of ingredients enumerated above, as required, followed by sterilization microfiltration.

Generally, dispersions are prepared by incorporating the active compound into a sterile vehicle that contains a basic dispersion medium and the required other ingredients from those enumerated above. In the case of sterile powders for the preparation of sterile injectable solutions, the preferred methods of preparation are vacuum drying and freeze-drying (lyophilization) that yield a powder of the active ingredient plus any additional desired ingredient from a previously sterile-filtered solution thereof.

Dosage regimens are adjusted to provide the optimum desired response (e.g., a therapeutic response). For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the individuals to be treated; each unit contains a predetermined quantity of active compound calculated to produce the desired therapeutic effect in association with the required pharmaceutical carrier. The specification for the dosage unit forms used according to the present invention are dictated by and directly dependent on (a) the unique characteristics of the active compound and the particular therapeutic effect to be achieved, and (b) the limitations inherent in the art of compounding such an active compound for the treatment of sensitivity in individuals.

Examples of pharmaceutically-acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

For the therapeutic/pharmaceutical formulations, compositions according to the present invention include those suitable for enteral administration (such as oral or rectal) or parenteral administration (such as nasal, topical (including vaginal, buccal and sublingual)). The compositions may conveniently be presented in unit dosage form and may be prepared by any methods known in the art of pharmacy. The amount of active ingredient (in particular, the amount of a compound according to the present invention) which can be combined with a carrier material to produce a pharmaceutical composition (such as a single dosage form) will vary depending upon the individual being treated, and the particular mode of administration. The amount of active ingredient which can be combined with a carrier material to produce a single dosage form will generally be that amount of the composition which produces a therapeutic effect.

Generally, out of 100% (for the pharmaceutical formulations/compositions), the amount of active ingredient (in particular, the amount of the compound according to the present invention (or of the compound used in the present invention), optionally together with other therapeutically active agents, if present in the pharmaceutical formulations/compositions) will range from about 0.01% to about 99%, preferably from about 0.1% to about 70%, most preferably from about 1% to about 30%, wherein the reminder is preferably composed of the one or more pharmaceutically acceptable excipients.

The amount of active ingredient, e.g., a compound according to the present invention (or a compound used in the present invention), in a unit dosage form and/or when administered to an individual or used in therapy, may range from about 0.1 mg to about 1000 mg per unit, administration or therapy. In certain embodiments, a suitable amount of such active ingredient may be calculated using the mass or body surface area of the individual, including amounts of between about 1 mg/kg and 100 mg/kg, or between about 1 mg/m² and about 400 mg/m².

Actual dosage levels of the active ingredients in the pharmaceutical compositions according to the present invention may be varied so as to obtain an amount of the active ingredient which is effective to achieve the desired therapeutic response for a particular patient, composition, and mode of administration, without being toxic to the patient. The selected dosage level will depend upon a variety of pharmacokinetic factors including the activity of the particular compositions employed, the route of administration, the time of administration, the rate of excretion of the particular compound being employed, the duration of the treatment, other drugs, compounds and/or materials used in combination with the particular compositions employed, the age, sex, weight, condition, general health and prior medical history of the patient being treated, and like factors well known in the medical arts.

A physician or veterinarian having ordinary skill in the art can readily determine and prescribe the (e.g., therapeutically) effective amount of the pharmaceutical composition required. For example, the physician or veterinarian could start with doses of the compounds according to the present invention (or of the compounds used in the present invention) at levels lower than that required in order to achieve the desired therapeutic effect and gradually increase the dosage until the desired effect is achieved. In general, a suitable daily dose of a composition according to the present invention will be that amount of the compound which is the lowest dose effective to produce a therapeutic effect. Such an effective dose will generally depend upon the factors described above. It is preferred that administration be oral, intravenous, intramuscular, intraperitoneal, or subcutaneous, preferably administered proximal to the site of the target. If desired, the (e.g., therapeutically) effective daily dose of a pharmaceutical composition may be administered as two or more sub-doses administered separately at appropriate intervals throughout the day, optionally, in unit dosage forms. While it is possible for a compound according to the present invention (or for the compound used in the present invention) to be administered alone, it is preferable to administer the compound as a pharmaceutical formulation/composition.

For oral administration, the pharmaceutical composition according to the present invention can take the form of, for example, tablets or capsules prepared by conventional means with pharmaceutical acceptable excipients such as binding agents (e.g., pregelatinised maize starch, polyvinylpyrrolidone, hydroxypropyl methylcellulose), fillers (e.g., lactose, microcrystalline cellulose, calcium hydrogen phosphate), lubricants (e.g., magnesium stearate, talc, silica), disintegrants (e.g., potato starch, sodium starch glycolate), or wetting agents (e.g., sodium lauryl sulphate). Liquid preparations for oral administration can be in the form of, for example, solutions, syrups, or suspensions, or can be presented as a dry product for constitution with water or other suitable vehicle before use. Such liquid preparation can be prepared by conventional means with pharmaceutically acceptable additives such as suspending agents (e.g., sorbitol, syrup, cellulose derivatives, hydrogenated edible fats), emulsifying agents (e.g., lecithin, acacia), non-aqueous vehicles (e.g., almond oil, oily esters, ethyl alcohol, fractionated vegetable oils), preservatives (e.g., methyl or propyl-p-hydroxycarbonates, sorbic acids). The preparations can also contain buffer salts, flavouring, colouring and sweetening agents as deemed appropriate. Preparations for oral administration can be suitably formulated to give controlled release of the pharmaceutical composition of the invention.

In one embodiment, the compound is orally administered in a concentration of, for example, at most 100 mg/kg body weight.

In one embodiment, the compound is parenterally administered (e.g., intravenously, intramuscularly, or subcutaneously), in a concentration of, for example, at most 10 mg/kg body weight.

The pharmaceutical composition can be formulated as a suppository, with traditional binders and carriers such as triglycerides. Oral formulation can include standard carriers such as pharmaceutical grades of mannitol, lactose, starch, magnesium stearate, sodium saccharine, cellulose, magnesium carbonate, etc.

For administration by inhalation, the pharmaceutical composition according to the present invention is conveniently delivered in the form of an aerosol spray presentation from a pressurized pack or a nebulizer, with the use of a suitable propellant (e.g., dichlorodifluoromethane, trichlorofluoromethane, dichlorotetrafluoroethane, carbon dioxide, nitrogen, or other suitable gas). In the case of a pressurized aerosol, the dosage unit can be determined by providing a valve to deliver a metered amount. Capsules and cartridges of, for example, gelatine, for use in an inhaler or insufflator can be formulated containing a powder mix of the pharmaceutical composition according to the present invention and a suitable powder base such as lactose or starch.

The pharmaceutical composition according to the present invention can be formulated for parenteral administration by injection, for example, by bolus injection or continuous infusion. In one embodiment, the compounds or compositions according to the present invention may be administered by slow continuous infusion over a long period, such as more than 24 hours, in order to reduce toxic side effects. The administration may also be performed by continuous infusion over a period of from 2 to 24 hours. Such regimen may be repeated one or more times as necessary, for example, after 6 months.

In yet another embodiment, the compounds or compositions according to the present invention are administered by maintenance therapy, such as, e.g., once a week for a period of 6 months or more.

Formulations for injection can be presented in units dosage form (e.g., in phial, in multi-dose container), and with an added preservative. The pharmaceutical composition according to the present invention can take such forms as suspensions, solutions or emulsions in oily or aqueous vehicles, and can contain formulatory agents such as suspending, stabilizing, or dispersing agents. Alternatively, the agent can be in powder form for constitution with a suitable vehicle (e.g., sterile pyrogen-free water) before use. Typically, compositions for intravenous administration are solutions in sterile isotonic aqueous buffer. Where necessary, the composition can also include a solubilizing agent and a local anesthetic such as lignocaine to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients can be mixed prior to administration.

Compositions according to the present invention which are suitable for vaginal administration also include pessaries, tampons, creams, gels, pastes, foams or spray formulations containing such carriers as are known in the art to be appropriate. Dosage forms for the topical or transdermal administration of compositions according to the present invention include powders, sprays, ointments, pastes, creams, lotions, gels, solutions, patches and inhalants. The active compound may be mixed under sterile conditions with a pharmaceutically acceptable carrier, and with any preservatives, buffers, or propellants which may be required.

Therapeutic/pharmaceutical compositions can be administered with medical devices known in the art. For example, in a preferred embodiment, a therapeutic/pharmaceutical composition according to the present invention can be administered with a needleless hypodermic injection device, such as the devices disclosed in U.S. Pat. Nos. 5,399,163; 5,383,851; 5,312,335; 5,064,413; 4,941,880; 4,790,824; or U.S. Pat. No. 4,596,556. Examples of well-known implants and modules useful in the present invention include those described in: U.S. Pat. No. 4,487,603, which discloses an implantable micro-infusion pump for dispensing medication at a controlled rate; U.S. Pat. No. 4,486,194, which discloses a therapeutic device for administering medicaments through the skin; U.S. Pat. No. 4,447,233, which discloses a medication infusion pump for delivering medication at a precise infusion rate; U.S. Pat. No. 4,447,224, which discloses a variable flow implantable infusion apparatus for continuous drug delivery; U.S. Pat. No. 4,439,196, which discloses an osmotic drug delivery system having multi-chamber compartments; and U.S. Pat. No. 4,475,916, which discloses an osmotic drug delivery system.

Many other such implants, delivery systems, and modules are known to those skilled in the art. In certain embodiments, the compounds according to the present invention can be formulated to ensure proper distribution in vivo. For example, the blood-brain barrier (BBB) excludes many highly hydrophilic compounds. To ensure that the compounds according to the present invention cross the BBB (if desired), they can be formulated, for example, in liposomes. For methods of manufacturing liposomes, see, e.g., U.S. Pat. Nos. 4,522,811; 5,374,548; and 5,399,331. The liposomes may comprise one or more moieties which are selectively transported into specific cells or organs, and thus enhance targeted drug delivery (see, e.g., V. V. Ranade (1989) J. Clin. Pharmacol. 29: 685). Exemplary targeting moieties include folate or biotin (see, e.g., U.S. Pat. No. 5,416,016 to Low et al.); mannosides (Umezawa et al., (1988) Biochem. Biophys. Res. Commun. 153: 1038); antibodies (P. G. Bloeman et al. (1995) FEBS Lett. 357: 140; M. Owais et al. (1995) Antimicrob. Agents Chemother. 39: 180); and surfactant protein A receptor (Briscoe et al. (1995) Am. J. Physiol. 1233: 134).

In one embodiment, the compounds according to the present invention (or the compounds used in the present invention) are formulated in liposomes. In a more preferred embodiment, the liposomes include a targeting moiety. In a most preferred embodiment, the compounds in the liposomes are delivered by bolus injection to a site proximal to the desired area. Such liposome-based composition should be fluid to the extent that easy syringability exists, should be stable under the conditions of manufacture and storage and should be preserved against the contaminating action of microorganisms such as bacteria and fungi.

A “therapeutically effective dosage” for therapy/treatment can be measured by objective responses which can either be complete or partial. A complete response (CR) is defined as no clinical, radiological or other evidence of a condition, disorder or disease. A partial response (PR) results from a reduction in disease of greater than 50%. Median time to progression is a measure that characterizes the durability of the objective tumor response.

A “therapeutically effective dosage” for therapy/treatment can also be measured by its ability to stabilize the progression of a condition, disorder or disease. The ability of a compound to inhibit one or more protein kinases or to reduce the viability of cells associated with a proliferative disorder, such as cancer cells can be evaluated by using appropriate in-vitro assays known to the skilled practitioner, such as those described herein (in particular in the Examples below). Alternatively, the properties of a compound described in the present invention can be evaluated by examining the ability of the compound in appropriate animal model systems known to the skilled practitioner such as those described herein (in particular in the Examples below). A therapeutically effective amount of a compound according to the present invention can cure, heal, alleviate, relieve, alter, remedy, ameliorate, improve or affect the condition, disorder or disease or the symptoms of the condition, disorder or disease or the predisposition toward the condition, disorder or disease in an individual. One of ordinary skill in the art would be able to determine such amounts based on such factors as the individual's size, the severity of the individual's symptoms, and the particular composition or route of administration selected.

The pharmaceutical composition according to the invention can also, if desired, be presented in a pack, or dispenser device which can contain one or more (e.g., unit) dosage forms containing the active compound. The pack can for example comprise metal or plastic foil, such as blister pack. The pack or dispenser device can be accompanied with a leaflet or other information; in particular, that describing (either to the patient and/or the administering physician) salient information or details on the pharmaceutical composition contained in the package, such as how to administer, recommended dosages, safety and/or side-effect information.

In a particular embodiment, a pharmaceutical composition of the invention is formulated for oral administration, and in an alternative particular embodiment, a pharmaceutical composition of the invention is formulated for intravenous administration.

In one embodiment, a pharmaceutical composition of the invention is in unit dose form, and in particular may be in a unit dose form that is formulated for oral administration.

Each of such a unit dose form may comprise (e.g., it may contain) between 1 and 1000 mg of the compound, such as the kinase inhibitor of the first aspect (or a compound used in the invention) (e.g., a kinase inhibitor having the general formula (I), (I), (II), (III), (IV), (V), (Va), (VI), (VII), (VIIa), (VIII), (IX), (X), (XI) or (XII), or a solvate, salt (in particular a pharmaceutically acceptable salt), N-oxide (in particular, an N-oxide of R^{1a} and/or R^{6″}), complex, polymorph, crystalline form, racemic mixture, diastereomer, enantiomer, tautomer, conformer, isotopically labeled form, prodrug and/or having at least one derivatized hydroxyl group, as specified above, or a solvate, salt, N-oxide, complex, polymorph, crystalline form, racemic mixture, diastereomer, enantiomer, tautomer, conformer, isotopically labeled form or combination thereof), or combination thereof).

In particular of such embodiments, a pharmaceutical composition of the invention that is in unit dose form (and in particular one be in a unit dose form that is formulated for oral administration) may comprise (e.g., it may contain)—for each unit dose form—about an amount of such compound between about 1 mg and 1000 mg.

In one particular embodiment, the pharmaceutical composition of the invention is (e.g., is formed as) a tablet, caplet or capsule; suitably the pharmaceutical composition of the invention (e.g., a unit dose form thereof) is a caplet. Methods to form (e.g., manufacture) tablets and caplets are, for example, described elsewhere herein.

Suitable excipients for the pharmaceutical compositions of the invention, in particular when formed as a tablet or caplet, include, and particular embodiments of such a pharmaceutical composition of the invention include those that further comprise one or more of the excipients disclosed herein.

### Therapeutic and Other Applications

In a third aspect, the present application provides a compound as specified above under the heading “Compounds” or a pharmaceutical composition as specified above under the heading “Pharmaceutical compositions” for use as a medicament, for example for use in therapy.

It is contemplated that a compound as specified above under the heading “Compounds” (or a pharmaceutical composition as specified above under the heading “Pharmaceutical compositions”) may be used for the inhibition of: (i) a kinase, such as one described herein, in particular SIK3, SIK2, SIK1, ABL/BCR-ABL, KIT, NEK2, BRAF. CSF1R, HCK, TEC-family kinases (eg BTK, TXK or ITK), AXL, BLK, TYRO3, MERTK, ZAP70, SYK, EGFR, and/or BRK and/or FLT3, LCK, PHA2, EPHA4, ACK1, NEK11, WEE1, WNK2, Aurora-A, Aurora-B and TBK1; and/or (ii) cellular resistance to an (e.g., a cell-mediated) immune response. For example, in a related aspect the compound (especially, where the compound is one of the first aspect of the invention) or pharmaceutical composition (especially, where the pharmaceutical composition is one of the second aspect of the invention) can be used in a method for the treatment of a disease, disorder or condition in a subject (in particular a human patient), comprising administering to the subject the compound (or pharmaceutical composition), wherein the disease or condition is associated with such kinase.

In particular (and as further described in the fifth aspect below), it is contemplated that a compound as specified above under the heading “Compounds” (or a pharmaceutical composition as specified above under the heading “Pharmaceutical compositions”) may be used for the treatment of a proliferative disorder (such as MPAL) characterised by (or cells involved with the proliferative disorder characterised by), inter-alia, the presence of MEF2C protein (such as phosphorylated MEF2C protein and/or MEF2C protein as an active transcription factor), a human chromosomal translocation at 11q23, and/or a KMT2A fusion oncoprotein.

The compounds or the pharmaceutical compositions of the invention (or the compounds or the pharmaceutical compositions used in the invention) may be used for treatment alone or in conjunction with one or more additional therapeutic agents, for example in combination with those that are defined or disclosed elsewhere herein, and that include an immune checkpoint inhibitor (such as an inhibitor of PD1, PDLL, CTLA-4, LAG3 or IDO1, and in particular an immune checkpoint inhibitor selected from the list consisting of: nivolumab, relatlimab, ipilimumab and BMS-986205), TNF or an agonist of TNFR1- or TNFR2-signalling (preferably of TNFR1-signalling), adoptive cellular therapy including CART cells directed against a tumor antigen, vaccines including dendritic cell- (DC) based vaccination, or an agent that is capable of inducing or induces the exposure of the cells involved with the proliferative disorder to TNF or an agonist of TNFR1- or TNFR2-signalling, is administered to the subject.

Treatment including or utilising such compounds may be provided at home, the doctor's office, a clinic, a hospital's outpatient department, or a hospital. Treatment generally begins under medical supervision so that medical personnel can observe the treatment's effects closely and make any adjustments that are needed. The duration of the treatment depends on the age and condition of the patient, as well as how the patient responds to the treatment.

A person having a greater risk of developing a condition, disorder or disease may receive prophylactic treatment to inhibit or delay symptoms of the condition, disorder or disease.

The term “treatment” is known to the person of ordinary skill, and includes the application or administration of a therapeutic agent (e.g., a pharmaceutical composition containing said agent) or procedure to a patient or application or administration of a therapeutic agent (e.g., a pharmaceutical composition containing said agent) or procedure to a cell, cell culture, cell line, sample, tissue or organ isolated from a patient, who has a condition, disorder or disease, a symptom of the condition, disorder or disease or a predisposition toward a condition, disorder or disease, with the purpose to cure, heal, alleviate, relieve, alter, remedy, ameliorate, improve, affect or prevent the condition, disorder or disease, the symptoms of the condition, disorder or disease or the predisposition toward the condition, disorder or disease. Hence, the term “treatment” can include prophylactic treatment of a condition, disorder or disease, or the symptom of a condition, disorder or disease. A therapeutic agent, when used in treatment, includes the kinase inhibitors of the invention (or the compounds used in the fifth aspect of the present invention) and includes, but is not limited to, additional therapeutic agents that may be small molecules, peptides, peptidomimetics, polypeptides/proteins, antibodies, nucleotides such as DNA or RNA, cells, viruses, ribozymes, siRNA, and antisense oligonucleotides.

Accordingly, in one fourth aspect, and as may be further described, defined, claimed or otherwise disclosed herein, the present invention relates to a compound as specified under the heading “Compounds” (e.g., a kinase inhibitor having the general formula (I), (I), (II), (III), (IV), (V), (Va), (VI), (VII), (VIIa), (VIII), (IX), (X), (XI) or (XII), or a solvate, salt (in particular a pharmaceutically acceptable salt), N-oxide (in particular, N-oxides of R^{1a} and/or R⁶), complex, polymorph, crystalline form, racemic mixture, diastereomer, enantiomer, tautomer, conformer, isotopically labeled form, prodrug (in particular a prodrug having at least one derivatized hydroxyl group, as specified above, or a solvate, salt, N-oxide, complex, polymorph, crystalline form, racemic mixture, diastereomer, enantiomer, tautomer, conformer, isotopically labeled form or combination thereof), or combination thereof) for use in a treatment of a proliferative disorder in a subject.

In another fourth aspect, and as may be further described, defined, claimed or otherwise disclosed herein, the present invention relates to a pharmaceutical composition as described above (e.g., one comprising a compound as specified under the heading “Compounds” (e.g., a kinase inhibitor having the general formula (I), (I), (II), (III), (IV), (V), (Va), (VI), (VII), (VIIa), (VIII), (IX), (X), (XI) or (XII), or a solvate, salt (in particular a pharmaceutically acceptable salt), N-oxide (in particular, N-oxides of R^{1a} and/or R⁶), complex, polymorph, crystalline form, racemic mixture, diastereomer, enantiomer, tautomer, conformer, isotopically labeled form, prodrug (in particular a prodrug having at least one derivatized hydroxyl group, as specified above, or a solvate, salt, N-oxide, complex, polymorph, crystalline form, racemic mixture, diastereomer, enantiomer, tautomer, conformer, isotopically labeled form or combination thereof), or combination thereof)) for use in a treatment of a proliferative disorder in a subject.

In a related fourth aspect, and as may be further described, defined, claimed or otherwise disclosed herein, the present invention relates to a method for the treatment of a proliferative disorder in a subject, comprising administering to the subject (e.g., a therapeutically effective amount of): (X) a compound as specified under the heading “Compounds” (e.g., a kinase inhibitor having the general formula (I), (I), (II), (III), (IV), (V), (Va), (VI), (VII), (VIIa), (VIII), (IX), (X), (XI) or (XII), or a solvate, salt (in particular a pharmaceutically acceptable salt), N-oxide (in particular, N-oxides of R^{1a}) and/or R⁶, complex, polymorph, crystalline form, racemic mixture, diastereomer, enantiomer, tautomer, conformer, isotopically labeled form, prodrug (in particular a prodrug having at least one derivatized hydroxyl group, as specified above, or a solvate, salt, N-oxide, complex, polymorph, crystalline form, racemic mixture, diastereomer, enantiomer, tautomer, conformer, isotopically labeled form or combination thereof), or combination thereof); or (Y) a pharmaceutical composition as described above (e.g., one comprising a compound as specified under the heading “Compounds” (e.g., a kinase inhibitor having the general formula (I), (I), (II), (III), (IV), (V), (Va), (VI), (VII), (VIIa), (VIII), (IX), (X), (XI) or (XII), or a solvate, salt (in particular a pharmaceutically acceptable salt), N-oxide (in particular, N-oxides of R^{1a} and/or R⁶), complex, polymorph, crystalline form, racemic mixture, diastereomer, enantiomer, tautomer, conformer, isotopically labeled form, prodrug (in particular a prodrug having at least one derivatized hydroxyl group, as specified above, or a solvate, salt, N-oxide, complex, polymorph, crystalline form, racemic mixture, diastereomer, enantiomer, tautomer, conformer, isotopically labeled form or combination thereof), or combination thereof)).

In another related fourth aspect, and as may be further described, defined, claimed or otherwise disclosed herein, the present invention relates to a use of a compound as specified under the heading “Compounds” (e.g., a kinase inhibitor having the general formula (I), (I), (II), (III), (IV), (V), (Va), (VI), (VII), (VIIa), (VIII), (IX), (X), (XI) or (XII), or a solvate, salt (in particular a pharmaceutically acceptable salt), N-oxide (in particular, N-oxides of R^{1a} and/or R⁶), complex, polymorph, crystalline form, racemic mixture, diastereomer, enantiomer, tautomer, conformer, isotopically labeled form, prodrug (in particular a prodrug having at least one derivatized hydroxyl group, as specified above, or a solvate, salt, N-oxide, complex, polymorph, crystalline form, racemic mixture, diastereomer, enantiomer, tautomer, conformer, isotopically labeled form or combination thereof), or combination thereof) for the manufacture of a medicament for the treatment of a proliferative disorder in a subject.

In such fourth aspects, the treatment of such use or method comprises administering to the subject (e.g., a therapeutically effective amount of) a compound or pharmaceutical composition of the invention.

In certain embodiments of the therapy-related aspects of the present invention, the disease, disorder or condition is associated with a kinase, such as one or more disclosed herein; for example one or more of such kinases as disclosed herein being associated with a disease, disorder or condition.

Furthermore, in a fifth aspect, and as may be further described, defined, claimed or otherwise disclosed herein, the present invention relates to a compound for use, or a pharmaceutical composition for use, in a treatment of a proliferative disorder in a subject, the treatment comprising administering the compound or the pharmaceutical composition to the subject, wherein the compound is selected from a compound of the first aspect (e.g., a kinase inhibitor having the general formula (I), (I), (II), (III), (IV), (V), (Va), (VI), (VII), (VIIa), (VIII), (IX), (X), (XI) or (XII), or a solvate, salt (in particular a pharmaceutically acceptable salt), N-oxide (in particular, N-oxides of R^{1a} and/or R⁶), complex, polymorph, crystalline form, racemic mixture, diastereomer, enantiomer, tautomer, conformer, isotopically labeled form, prodrug (in particular a prodrug having at least one derivatized hydroxyl group, as specified above, or a solvate, salt, N-oxide, complex, polymorph, crystalline form, racemic mixture, diastereomer, enantiomer, tautomer, conformer, isotopically labeled form or combination thereof), or combination thereof); and wherein the proliferative disorder is selected from one or more of (a) to (y): (a) a proliferative disorder characterised by (or cells involved with the proliferative disorder characterised by) the presence of (or an amount of) myocyte enhancer factor 2C (MEF2C) protein, such as of phosphorylated MEF2C protein and/or of MEF2C protein as an active transcription factor; preferably wherein the proliferative disorder is further characterised by (or cells involved with the proliferative disorder are further characterised by) the presence of (or an amount of phosphorylated histone deacetylase 4 (HDAC4) protein, such as of HDAC4 protein phosphorylated by SIK3; and/or (0) a proliferative disorder characterised by: (i) the presence of a human chromosomal translocation at 11q23; (ii) the presence of a rearrangement of the lysine methyltransferase 2A (KMT2A) gene; (iii) the presence of (or an amount of) an KMT2A fusion oncoprotein; and/or (iv) the presence of a mutation in the K-RAS proto-oncogene GTPase (KRAS) gene and/or in the RUNX family transcription factor 1 (RUNX1) gene; and/or (y) a mixed phenotype acute leukaemia (MPAL). In a related aspect, the present invention provides a method for the treatment of a proliferative disorder in a subject, comprising administering to the subject a compound or pharmaceutical composition as defined in the fifth aspect, wherein the proliferative disorder is as defined in the fifth aspect.

In one particular embodiment of such aspects, the subject is a human, suitably an adult human; for example, a human that is 18 (or 16) years old or older.

As an alternative to such embodiments, the subject treated is a paediatric human, such as being younger than about 18 (or 16) years old.

In one embodiment of such aspects, the treatment comprises administering to an adult human subject in need thereof an amount of a compound of the invention (for example, as comprised in a pharmaceutical composition).

In one alternative embodiment, the treatment comprises administering to a paediatric human subject in need thereof an amount of a compound of the invention (or of a compound used in the invention).

The disease, disorder or a condition, in the context of the herein described invention, is, in certain embodiments, a proliferative disorder (including a condition or symptom associated with such disorder).

A “proliferative disorder” refers to a disorder characterised by abnormal proliferation of cells. A proliferative disorder does not imply any limitation with respect to the rate of cell growth, but merely indicates loss of normal controls that affect growth and cell division. Thus, in some embodiments, cells of a proliferative disorder can have the same cell division rates as normal cells but do not respond to signals that limit such growth. Within the ambit of “proliferative disorder” is neoplasm or tumour, which is an abnormal growth of tissue or cells. Cancer is art understood, and includes any of various malignant neoplasms characterised by the proliferation of cells that have the capability to invade surrounding tissue and/or metastasise to new colonisation sites. Proliferative disorders include cancer, atherosclerosis, rheumatoid arthritis, idiopathic pulmonary fibrosis and cirrhosis of the liver. Non-cancerous proliferative disorders also include hyperproliferation of cells in the skin such as psoriasis and its varied clinical forms, Reiter's syndrome, pityriasis rubra pilaris, and hyperproliferative variants of disorders of keratinisation (e.g., actinic keratosis, senile keratosis), scleroderma, and the like.

In more particular embodiments, the proliferative disorder is a cancer or tumour, in particular a solid tumour (including a condition or symptom associated with such cancer or tumour). Such proliferative disorders including but not limited to head and neck cancer, squamous cell carcinoma, multiple myeloma, solitary plasmacytoma, renal cell cancer, retinoblastoma, germ cell tumours, hepatoblastoma, hepatocellular carcinoma, melanoma, rhabdoid tumour of the kidney, Ewing Sarcoma, chondrosarcoma, any haemotological malignancy (e.g., chronic lymphoblastic leukemia, chronic myelomonocytic leukemia, acute lymphoblastic leukemia, acute lymphocytic leukemia, acute myelogenous leukemia, acute myeloblasts leukemia, chronic myeloblastic leukemia, Hodgkin's disease, non-Hodgkin's lymphoma, chronic lymphocytic leukemia, chronic myelogenous leukemia, myelodysplastic syndrome, hairy cell leukemia, mast cell leukemia, mast cell neoplasm, follicular lymphoma, diffuse large cell lymphoma, mantle cell lymphoma, marginal zone lymphoma, Burkitt Lymphoma, mycosis fungoides, seary syndrome, cutaneous T-cell lymphoma, peripheral T cell lymphoma, chronic myeloproliferative disorders, myelofibrosis, myeloid metaplasia, systemic mastocytosis), and central nervous system tumours (eg, brain cancer, glioblastoma, non-glioblastoma brain cancer, meningioma, pituitary adenoma, vestibular schwannoma, a primitive neuroectodermal tumour, medulloblastoma, astrocytoma, anaplastic astrocytoma, oligodendroglioma, ependymoma and choroid plexus papilloma), myeloproliferative disorders (eg, polycythemia vera, thrombocythemia, idiopathic myelfibrosis), soft tissue sarcoma, thyroid cancer, endometrial cancer, carcinoid cancer, or liver cancer.

In a particular embodiment, the various aspects of the invention relate to (for example the compounds or the pharmaceutical compositions of the invention are used in) treatments for proliferative disorders that include those described herein. Accordingly, in such embodiments the proliferative disorder may be a cancer or tumour.

In certain embodiments of the various aspects of the invention, the proliferative disorder is selected from one or more of:
a mixed phenotype acute leukaemia (MPAL), especially MPAL with MLL (KMT2A) rearrangement; and/or
a proliferative disorder characterised by (or cells involved with the proliferative disorder characterised by): (i) the presence of a human chromosomal translocation at 11q23; (ii) the presence of a rearrangement of the lysine methyltransferase 2A (KMT2A) gene; (iii) the presence of a KMT2A fusion oncoprotein; and/or (iv) the presence of a mutation in the K-RAS proto-oncogene GTPase (KRAS) gene and/or in the RUNX family transcription factor 1 (RUNX1) gene; and/or
a proliferative disorder characterised by (or cells involved with the proliferative disorder characterised by) the presence of myocyte enhancer factor 2C (MEF2C) protein.

In particular embodiments, the proliferative disorder may be mixed phenotype acute leukaemia (MPAL); also known as “mixed lineage leukaemia” (MLL). MPAL is a very aggressive blood cancer that predominantly occurs in paediatric patients and, unlike other types of childhood acute leukaemias, has a dismal prognosis (reviewed by Slany 2009, Haematologica 94:984). One form of MPAL is characterised by the presence of lysine methyltransferase 2A (KMT2A) fusion proteins (also known as MLLfusion proteins) that are the result of chromosomal translocations affecting the KMT2A gene (also known as the MLL1 gene) at 11q23. This KMT2A/MLL rearrangement is the second most frequent genetic lesion in MPAL (MPAL MLL+). These 11q23 translation events juxtapose the amino-terminus of the histone methyltransferase KMT2A with a variety of different (translocation) fusion partners that destroy normal histone methyltransferase function of KMT2A and replace it by heterologous functions contributed by the (translocation) fusion partner. The resulting protein chimeras are transcriptional regulators that take control of targets normally controlled by KMT2A. In particular, the transcription factor MEF2C can be controlled by KMT2A and is described as an oncogene in childhood acute leukaemias. MEF2C expression is associated with KMT2A fusion gene rearrangement in AML (Schwieger et al 2009, Blood 114:2476), and MEF2C expression defines a subset of AML patients with poor survival outcome (Lazlo et al 2015, J Hematol & Oncol 8:115). In one of such embodiments, the proliferative disorder is MPAL with MLL (KMT2A) rearrangement.

In particular embodiments, the proliferative disorder may be characterised by (or cells involved with the proliferative disorder may be characterised by) the presence of a human chromosomal translocation at 11q23, such as a human chromosome translocation selected from the group consisting of: t(4,11), t(9,11), t(11,19), t(10,11) and t(6,11), in particular t(4;11)(q21;q23) [TPG: AF4], t(9;11)(p22;q23) [TPG: AF9], t(11;19)(q23;p13.3) [TPG: ENL], ins(10;11)(p12;q23q13) [TPG: AF10], t(11;19)(q23;p13.1) [TPG: ELL] and t(6;11)(q27;q23) [TPG: AF6]. In other embodiments, the human chromosomal translocation may be any of those identified in Table 2 of Meyer et al 2018.

The lysine methyltransferase 2A (KMT2A) gene on human chromosome 11q23 (previously now as mix-lineage leukaemia 1 gene, MLL1) can be disrupted by such translocations; producing a fusion with one of more than 90 (known) translocation partner genes (Meyer et al 2018, Leukemia 32:273). The majority of leukaemias result from KMT2A fusions with one of about six common (translocation) partner genes (as reviewed by Winters & Bernt 2017, Front Ped 5:4), with nine specific gene fusions accounting for more than 90% of all illegitimate recombinations of the KMT2A fusions (Meyer et al 2018). Approximately 10% or all leukaemias harbour such translocations (and hence, KMT2A-fusion genes).

In certain embodiments, the proliferative disorder may be characterised by (or cells involved with the proliferative disorder may be characterised by) the presence of a rearrangement of the lysine methyltransferase 2A (KMT2A) gene, and/or the presence of a KMT2A fusion oncoprotein. For example, the KMT2A fusion oncoprotein may be present at an amount (eg a quantitative amount), such as an amount that is in excess of physiological amount (eg, for that cell type and/or that time/stage), including from expression or over-expression of the protein. In another embodiment, such protein may be present at an amount (eg a quantitative amount) that is in excess of a threshold amount or is an outlier from a reference distribution of amounts of such protein. In particular of such embodiments, the rearrangement of the KMT2A gene comprises, or the KMT2A fusion oncoprotein is expressed from a rearrangement that comprises, a fusion of the KMT2A gene with a translocation partner gene (TPG) selected from the group consisting of: AF4, AF9, ENL, AF10, ELL and AF6, in particular selected from the group consisting of AF4, AF9 and ENL. Other TPGs can include EPS15 or AF1Q, or any other any of those TPGs identified in lines 10 to 20, 21 to 30, 31 to 40, 41 to 50, 51 to 60, 61 to 70, 71 to 80, 81, 82 and/or 83 or Table 1 of Meyer et al 2018.

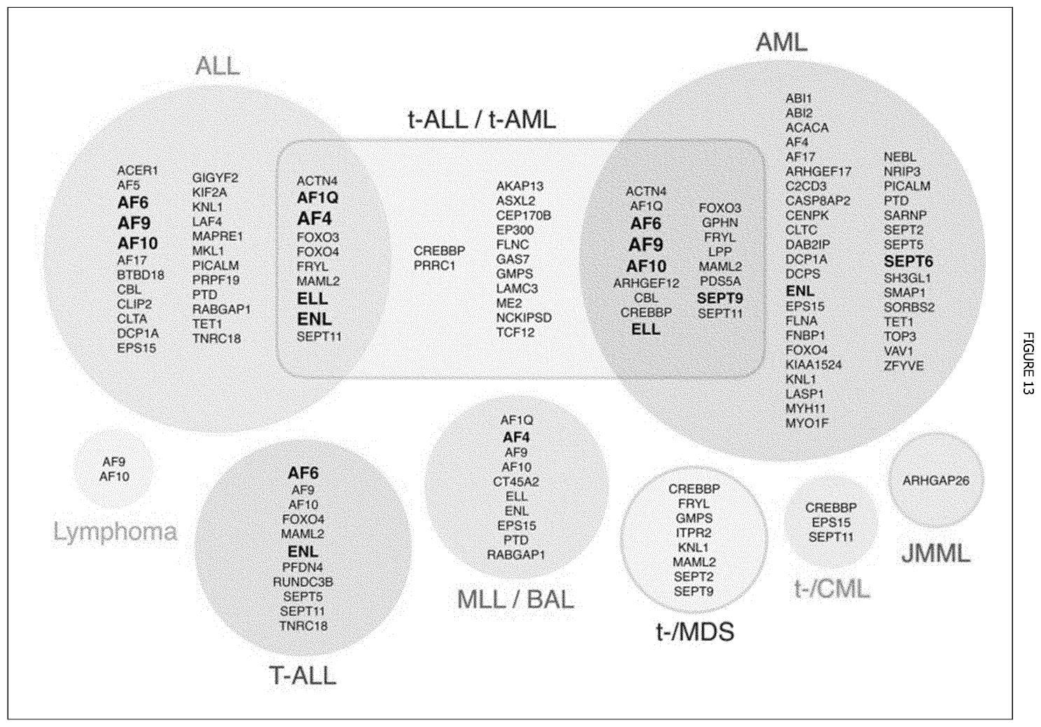

Certain TPGs can be associated with certain proliferative disorders, and in particular embodiments herein, the proliferative disorder and TPG of the KMT2A gene is one selected from the group shown in FIG. 13 (from Meyer et al 2018).

Tarumoto and co-workers (2018, Mol Cell 69:1017) showed that MEF2C activity in AML is driven by SIK3-phosphorylation of HDAC4, and that SIK3 knock-out or chemical inhibition with the small molecule tool compound HG-9-91-01 strongly decreases viability of several MPAL-associated AML cell lines (including MOLM-13 and MV4-11): because cytoplasmic retention of SIK3-phosphorylated HDAC4 regulates MEF2C activity, by preventing nuclear-located (un-phosphorylated) HDAC4 acting as a repressive cofactor of MEF2C, a transaction factor of tumour survival/maintenance genes associated with AML proliferation (FIG. 2).

Accordingly, in certain embodiments, a proliferative disorder may be one (or more) characterised by (or cells involved with the proliferative disorder may be characterised by) the presence of myocyte enhancer factor 2C (MEF2C) protein. For example, the MEF2C protein may be present at an amount (eg a quantitative amount), such as an amount that is in excess of physiological amount (eg, for that cell type and/or that time/stage), including from expression or over-expression of the protein. In another embodiment, such protein may be present at an amount (eg a quantitative amount) that is in excess of a threshold amount or is an outlier from a reference distribution of amounts of such protein. In particular of such embodiments, the MEF2C protein is a phosphorylated MEF2C protein, such as one phosphorylated by a MARK kinase (Vakoc & Kentis 2018, Oncotarget 9:32276), such as MEF2C protein phosphorylated at S222. The MEF2C protein may be one that acts as (eg, is) an active transcription factor. In particular embodiments, the proliferative disorder may be further characterised by (or cells involved with the proliferative disorder may be further characterised by) the presence of histone deacetylase 4 (HDAC4) protein, preferably in the nucleus of a cell and/or of phosphorylated HDAC4 protein (eg, HDAC4 protein phosphorylated by SIK3). Any of such HDAC4 proteins may be present at an amount (eg a quantitative amount), such as an amount that is in excess of physiological amount (eg, for that cell type and/or that time/stage), including from expression or over-expression of the protein. In another embodiment, such protein may be present at an amount (eg a quantitative amount) that is in excess of a threshold amount or is an outlier from a reference distribution of amounts of such protein.

Furthermore, Tarumoto and co-workers (2020, Blood 135:56) noted that multiple myeloma and AML cell lines express the highest levels of MEF2C and SIK3 when compared to other cancer cell lines. In their analysis of 162 genomically characterised human AML samples in TCGA1, they found that MEF2C expression was correlated, not only with the presence of MLL (11q23) translocations, but was also correlated with K-RAS proto-oncogene GTPase (KRAS) mutations and RUNX family transcription factor 1 (RUNX1) mutations.

Therefore, in certain embodiments the proliferative disorder may be characterised by (or cells involved with the proliferative disorder may be characterised by) the presence of a mutation in the KRAS gene and/or in the RUNX1 gene.

Another form of of MPAL is characterised by a BCR/ABL rearrangement. MPAL with t(9;22)(q34;q11.2) (or BCR/ABL1 rearrangement) is considered as a separate entity (Arber et al 2016, Blood 127:2391). The t(9;22)(q34;q11.2 translocation results in a BCR/ABL1 fusion gene located on the Philadelphia chromosome (Ph), causing a constitutively active BCR/ABL1 tyrosine kinase.

Hence, in another certain embodiments of the various aspects of the invention, the proliferative disorder is selected from one or more of:
a mixed phenotype acute leukaemia (MPAL), in particular MPAL with BCR/ABL1 fusion gene; and/or
a proliferative disorder characterised by (or cells involved with the proliferative disorder characterised by): (i) the presence of a human chromosomal translocation t(9;22)(q34;q11.2); (ii) the presence of a BCR/ABL1 rearrangement gene; (iii) the presence of a BCR/ABL1 fusion oncoprotein.

Accordingly, and in a further aspect, the invention relates to a compound or a pharmaceutical composition for use in a treatment of a proliferative disorder in a subject, the treatment comprising administering the compound or the pharmaceutical composition to the subject, wherein the compound is selected from the following compounds (a) or (b), or the pharmaceutical composition comprises such a compound and, optionally, a pharmaceutically acceptable excipient: (a) a compound of the first aspect (e.g., a kinase inhibitor having the general formula (I), (I), (II), (III), (IV), (V), (Va), (VI), (VII), (VIIa), (VIII), (IX), (X), (XI) or (XII), or a solvate, salt (in particular a pharmaceutically acceptable salt), N-oxide (in particular, N-oxides of R^{1a} and/or R⁶), complex, polymorph, crystalline form, racemic mixture, diastereomer, enantiomer, tautomer, conformer, isotopically labeled form, prodrug (in particular a prodrug having at least one derivatized hydroxyl group, as specified above, or a solvate, salt, N-oxide, complex, polymorph, crystalline form, racemic mixture, diastereomer, enantiomer, tautomer, conformer, isotopically labeled form or combination thereof), or combination thereof), such as any embodiment thereof as described above; or (b) ARN-3261 (Vankayalapati et al 2017, AACR Cancer Res 77(13 Suppl):Abstract nr LB-296; U.S. Pat. Nos. 9,260,426, 9,890,153, 9,951,062).

In a related aspect, the invention relates to a method for the treatment of a proliferative disorder in a subject, comprising administering to the subject such a compound or pharmaceutical composition.

In one particular embodiments of such further and related aspects, the proliferative disorder is selected from one or more of (X) to (Z):
(X) a proliferative disorder characterised by (or cells involved with the proliferative disorder characterised by) the presence of (and/or an amount of) myocyte enhancer factor 2C (MEF2C) protein, such as of phosphorylated MEF2C protein and/or of MEF2C protein as an active transcription factor; preferably wherein the proliferative disorder is further characterised by (or cells involved with the proliferative disorder are further characterised by) the presence of (and/or an amount of) phosphorylated histone deacetylase 4 (HDAC4) protein, such as of HDAC4 protein phosphorylated by SIK3; and/or
(Y) a proliferative disorder characterised by (or cells involved with the proliferative disorder characterised by): (i) the presence of a human chromosomal translocation at 11q23; (ii) the presence of a rearrangement of the lysine methyltransferase 2A (KMT2A) gene; (iii) the presence of (and/or an amount of) a KMT2A fusion oncoprotein; and/or (iv) the presence of a mutation in the K-RAS proto-oncogene GTPase (KRAS) gene and/or in the RUNX family transcription factor 1 (RUNX1) gene; and/or
(Z) a mixed phenotype acute leukaemia (MPAL), especially MPAL with MLL (KMT2A) rearrangement.

In another particular embodiments of such further and related aspects, the proliferative disorder is selected from one or more of (X′) an (Y′):
(X′) a mixed phenotype acute leukaemia (MPAL), especially MPAL with BCR/ABL1 fusion gene; and/or
(Y′) a proliferative disorder characterised by (or cells involved with the proliferative disorder characterised by): (i) the presence of a human chromosomal translocation t(9;22)(q34;q11.2); (ii) the presence of a BCR/ABL1 rearrangement gene; (iii) the presence of a BCR/ABL1 fusion oncoprotein.

In further embodiments of such aspects, the compound is one having a formula of (I), of (Ia) or of (II), wherein L is a bond and R⁶ is a 5- (or 6)-membered monocyclic heteroaryl which contains at least one S ring atom and which is optionally substituted with one, two or three independently selected R⁷, in particular those embodiments where R⁶ is thienyl optionally substituted with one, two or three independently selected R⁷.

In particular of such embodiments, such R⁶ is substituted with one, two or three independently selected R⁷, including those embodiments wherein R⁷ is independently selected from the group consisting of halogen and C₁₋₂alkyl, wherein the C₁₋₂ alkyl is optionally substituted with one, two or three independently selected R³⁰.

In certain of embodiments of such aspects, the compound is of formula (Ia), such as one selected from the group consisting of those shown in Table A and Table B (and/or depicted in FIGS. 3 A and B). In other aspects, the compound can be one selected from the group consisting of AA1, AA3, AA5, AA6, AA7, AA11 and AA12, such as compound AA5, or a compound selected from compound AA3, AA6 and AA7 (such as AA7). In yet other aspects, the can be ARN-3261 (Vankayalapati et al 2017, AACR Cancer Res 77(13 Suppl):Abstract nr LB-296; U.S. Pat. Nos. 9,260,426, 9,890,153, 9,951,062).

In any of such aspects, the proliferative disorder is, for example, a cancer of a tumour, such as a cancer or tumour described elsewhere herein. In particular embodiments, the proliferative disorder is a haematopoietic malignancy. The proliferative disorder may be a lymphoid malignancy.

In particular of such embodiments, the proliferative disorder may be: (i) a myeloma, preferably multiple myeloma; or (ii) a leukaemia, preferably an acute myeloid leukaemia (AML) or an acute lymphoblastic leukaemia (ALL), more preferably T cell acute lymphoblastic leukaemia (T-ALL), an MLL-AML or an MLL-ALL. In other embodiments, the proliferative disorder may be one selected from the group set out in FIG. 13.

A subject for treatment in connection with such aspects may, suitably, be a human paediatric patient; for example, a human individual of less than about 18 years of age (or 16 years or age).

In further embodiments, the subject carries a KMT2A rearrangement (KMT2A-r). For example, the subject may be a patient suffering from a KMT2A-r leukaemia, especially a (eg, paediatric) human patient as described elsewhere herein.

In one particular embodiment, the cancer is a hematopoietic or lymphoid cancer.

In another particular embodiment, the cancer is a solid tumour, and in one such embodiment, the proliferative disorder is (eg, the subject suffers from, or is suspected of suffering from) a solid tumour being one of those described elsewhere herein.

As described elsewhere, a compound (or pharmaceutical composition) of the invention (or a compound used in the invention) may be administered to the subject (eg, as a combination therapy or regimen) with another medical procedure (eg, an additional therapeutic agent, such as described elsewhere herein, surgery or radiotherapy). Then such combination treatment regimen may comprise embodiments where such exposures/administrations are concomitant. In alternative embodiments such administrations may be sequential; in particular those embodiments where a compound (or pharmaceutical composition) of the invention (or a compound used in the invention) is administered before such other procedure. For example the compound (or pharmaceutical composition) may be sequentially administered within about 14 days of (eg before) the other procedure.

Such combination regimens can include the (eg further) administration to the subject of:
an immune checkpoint inhibitor.

Exemplary immune checkpoint inhibitor that may be comprise such combination therapy or regimen are described elsewhere, and include an antibody or small-molecule inhibitor of PD1, PDL1, CTLA-4, LAG3 or IDO1, and in particular such an immune checkpoint inhibitor may be one selected from the list consisting of: nivolumab, relatlimab, ipilimumab and BMS-986205, in particular nivolumab.

In other embodiments, the combination regimens can include the (eg further) administration to the subject of:
an immune-activator (eg, agonist) antibody, such as an antibody against OX40 (eg, Yang et al 2012, Blood 120:4533), 41BB, CD40 or ICOS (eg, Deng et al 2004, Hybrid Hybridomics 23:176), in particular those that increase TNF levels by stimulated/stimulating T cells; and/or
dendritic cell- (DC) based vaccination (eg, Lowe et al 2014, Oncoimmunology 3:e27589).

In one particular embodiment, the proliferative disorder (eg, in the subject) has progressed on (eg despite) standard therapy, or in anther embodiment, the subject may be unable to receive standard therapy, for example as the subject is intolerant thereto. In either of such embodiments, the subject may be so characterised (eg, stratified) as having progressed on standard therapy or being unable to receive (eg, is intolerant to) standard therapy.

As examples of standard therapy, may be immunotherapy such as an immune checkpoint inhibitor described herein.

### Sensitisation to Immune Responses and Inhibition of Kinases

The compounds of the invention can sensitise cells involved with a proliferative disorder to a cell-mediated immune response.

Accordingly, in one embodiment, a treatment comprising administering a compound (or a pharmaceutical composition) of the invention to the subject involves (eg, is mediated, is or supported) sensitising cells involved with the proliferative disorder (in the subject) to a cell-mediated immune response.

In an alternative embodiment, a treatment comprising administering a compound (or a pharmaceutical composition) of the invention to the subject involves (eg, is mediated, is or supported by) inhibiting a kinase involved in resistance to a cell-mediated immune response, such as inhibiting SIK3 (in the subject).

In a related embodiment, a treatment comprising administering a compound (or a pharmaceutical composition) of the invention to the subject involves (eg, is mediated, is or supported by) inhibiting a kinase involved in resistance to a cell-mediated immune response, such as inhibiting SIK3, and (for example, thereby) sensitising cells involved with the proliferative disorder (in the subject) to a cell-mediated immune response.

In a further aspect, and as may be further described, defined, claimed or otherwise disclosed herein, the invention relates to a method for the sensitisation of cells involved with a proliferative disorder to a cell-mediated immune response in the treatment of the proliferative disorder in a subject, the method comprising administering a compound (or a pharmaceutical composition) of the invention to the subject; and in another further aspect, and as may be further described, defined, claimed or otherwise disclosed herein, the invention relates to a method for the inhibition of a kinase (in the subject) involved in resistance to a cell-mediated immune response, such as inhibiting, in the treatment of a proliferative disorder in a subject, the method comprising administering a compound (or a pharmaceutical composition) of the invention to the subject.

In a related further aspect, and as may be further described, defined, claimed or otherwise disclosed herein, the invention relates to a compound (or a pharmaceutical composition) of the invention for use as a medicament for: (i) sensitising cells involved with a proliferative disorder (in the subject) to a cell-mediated immune response; and/or (ii) inhibiting a kinase involved in resistance to a cell-mediated immune response, such as inhibiting SIK (in the subject).

In yet a related further aspect, and as may be further described, defined, claimed or otherwise disclosed herein, the invention relates to a compound (or a pharmaceutical composition) of the invention for use as a medicament (eg an immuno-oncology medicament) sensitising cells involved with a proliferative disorder (such as a tumour or cancer) to a cell-mediated immune response, for example sensitising cells involved with a proliferative disorder (in the subject) to killing (cell-death) that may be induced by the cell-mediated immune response. An “immune-oncology” medicament is one that would be recognised by the person of ordinary skill, and includes a medicament that is intended to (eg, specifically designed to) enhance one or more components of the immune system of an organism (such as a human) towards cancerous or tumourous cells present in such organism. An immune-oncology medicament may be one (eg an antibody) that binds to an extrinsic immune (inhibitory) checkpoint molecule (such as one described elsewhere herein) and that (eg directly) suppresses T cell function against the cancerous or tumourous cells, or an immune-oncology medicament may be one that inhibits an immune regulator (such as SIK3, as in the present invention) that is intrinsic to the cancerous or tumourous cells where such intrinsic immune regulator does not actively (eg directly) suppress T cells but rather protects the tumour or cancer cells from an immune response via a resistance mechanism.

In particular embodiments of such aspects, the cells involved with a proliferative disorder (in the subject) may be sensitised to killing (cell-death) by (such as induced by) the cell-mediated immune response.

“Salt-inducible kinase 3” or “SIK3” (synonyms QSK and KIAA0999) is a member of a subfamily of serine/threonine protein kinases including SIK1, SIK2, and SIK3 that belong to an AMP-activated protein kinase (AMPK) family. A SIK3 protein in context of the invention is, typically, a protein kinase. Pertinent information on the human SIK3 protein is accessible on UniProt: Q9Y2K2 (Entry version 138 of 15 Mar. 2017) and a SIK3 protein in context of the invention has, preferably, an amino acid sequence shown in SIK3, Entry version 138 of 15 Mar. 2017 or Entry version 144 of 28 Mar. 2018, which sequences are incorporated herein by reference. SIK3 is a cytoplasmatic protein with serine/threonine kinase activity which is regulated through phosphorylation of a conserved threonine residue (position 163) in the T-loop of the kinase domain by the LKB1 complex; a phosphorylation which is reported as essential for catalytic activity of SIK3 (Lizcano, J. M. et al.; EMBO J. 23, 833-843 (2004)). For the purposes of the herein disclosed invention the term “phosphorylated SIK3” shall denote a SIK3 protein that is phosphorylated substantially as SIK3 protein can be (eg is) phosphorylated by LKB1, wherein preferably such phosphorylated SIK3 comprising a phosphor-threonine at amino acid position 163. A phosphorylated SIK3 in context of the invention is an SIK3 protein that is activated in its cell-biological context. At least four protein isoforms (SIK3-001 to SIK3-004) generated by alternative splicing of the SIK3 gene product are known. The human SIK3 gene is located at chromosomal position 11q23.3 (HGNC gene Symbol Acc: HGNC:29165), and is conserved in many species such as in chimpanzee, Rhesus monkey, dog, cow, mouse, rat, chicken, zebrafish, and frog. The term SIK3 in some embodiments of the invention may also pertain to variants of the human SIK3 protein having an amino acid sequence that is substantially identical to, or of at least 80%, preferably 85%, more preferably 90, 95, 96, 97, 98, 99, or 100% sequence identity to, the amino acid sequence of SIK3 as described above, as determined using, e.g., the “Blast 2 sequences” algorithm described by Tatusova & Madden 1999 (FEMS Microbiol Lett 174: 247-250), and which (preferably) retain biological activity identical or substantially identical to the respective reference SIK3 (eg to phosphorylate one or more class II (eg IIa) HDACs, such as HDAC4). Preferred variants of SIK3 protein comprise sequence variants thereof due to sequence polymorphism between and within populations of the respective species, as well as mutations compared to the wild-type sequence of SIK3 which are located in or in close proximity to the activity loop or activation loop (T-loop) of SIK3. A preferred variant of SIK3 protein is a SIK3 T163 mutation, such as a mutation affecting the activation of SIK3. In preferred embodiments a SIK3 protein of the invention is not a SIK1 (synonyms: SIK and SNF1LK) protein and/or is not a SIK2 (synonyms: QIK, KIAA0781 and SNF1LK2) protein. The amino acid sequence of human SIK1 (UniProt: P57059; entry version 168 of 15 Mar. 2017) and human SIK2 (UniProt: Q9HOK1; entry version 153 of 15 Mar. 2017) are incorporated herein by reference. The term SIK3 can mean, as applicable to the context (if not more specifically indicated), a SIK3 protein (such as one described above) or an mRNA molecule encoding such a SIK3 protein. The analogous meaning with respect of “SIK1” and “SIK2” is to be understood.

A compound being an “inhibitor of SIK3” (or “SIK3 inhibitor”) is any moiety that inhibits SIK3, which can mean inhibition of the activity of SIK3, especially of protein of SIK3, and in particular of phosphorylated SIK3. A SIK3 inhibitor may impair (eg, induces a decrease or reduction in) the efficiency, effectiveness, amount or rate of one or more activities of SIK3, such as one or more of those activities described herein, for example, the activity of SIK3 to phosphorylate class II (eg IIa) HDACs (eg HDAC4) and/or to sensitise a cell involved with a proliferative disorder to a cell-mediated immune response.

Such a SIK3 inhibiting moiety can act directly, for example, by binding to SIK3 and decreasing the amount or rate of one or more of the properties of SIK3 such as its function, in particular its ability to act as a kinase (eg to phosphorylate HDAC4), for example by reducing the activity of phosphorylated SIK3 in the cell.

Compounds being SIK3 inhibitors are described elsewhere herein, including those as may be characterised by the applicable functional and/or structural features set out herein.

In preferred embodiments, a “subject”, in particular, is also meant to include all mammals, including without limitation humans, but also non-human primates such as cynomolgus monkeys. It also includes dogs, cats, horses, sheep, goats, cows, rabbits, pigs and rodents (such as mice and rats). It will be appreciated that a particularly preferred subject according to the invention is a human subject, such as a human suffering from (or at risk of suffering from) a disorder, disease or condition, for example a human patient.

As used herein, “therapy” is synonymous with treating a disease, disorder or condition, which includes reducing symptoms of the disease, disorder or condition, inhibiting progression of the disease, disorder or condition, causing regression of the disease, disorder or condition and/or curing the disease, disorder or condition.

In preferred embodiments, a “treatment” in the present invention, and in particular, is also meant to include therapy, e.g. therapeutic treatment, as well as prophylactic or suppressive measures for a disease (or disorder or condition). Thus, for example, successful administration of a compound (or pharmaceutical composition) of the invention prior to onset of the disease results in treatment of the disease. “Treatment” also encompasses administration of a compound (or pharmaceutical composition) of the invention after the appearance of the disease in order to ameliorate or eradicate the disease (or symptoms thereof). Administration of a compound (or pharmaceutical composition) of the invention after onset and after clinical symptoms, with possible abatement of clinical symptoms and perhaps amelioration of the disease, also comprises treatment of the disease. Those “in need of treatment” include subjects (such as a human subject) already having the disease, disorder or condition, as well as those prone to or suspected of having the disease, disorder or condition, including those in which the disease, disorder or condition is to be prevented.

The cell that is sensitised to the cell-mediated immune response is, suitably, one involved with the proliferative disorder (eg, a cell associated with the proliferative disorder) (in the subject), which in certain embodiments such cell is one involved in the proliferative disorder (eg, a cell that is abnormally proliferating, such as one that is over-proliferating). For example, such cell may be a cell characterised by loss of normal controls that affect its growth and cell division, such as a cell of a neoplasm or tumour. In particular embodiments, such cell may be a cancerous cell or one that is derived form or is a cell of a cancer or tumour. In other embodiments, such cell may be skin cell, such as one showing hyperproliferation such as one involved in psoriasis, Reiter's syndrome, pityriasis rubra pilaris or scleroderma.

A cell may be “involved with a proliferative disorder” if, for example, it is associated therewith, such as it being a causative factor in such proliferative disorder or if it is affected by such proliferative disorder. In particular a cell is “involved with a proliferative disorder” if the cell is characterised by an abnormal proliferation such as abnormal cell growth or cell division, and if the abnormal cell growth or cell division is part of the pathology of, or causative for, the proliferative disease. A cell “involved with a proliferative disorder”, in those embodiments wherein the proliferative disorder is a tumour or cancer, can as a non-limiting example, be a tumour (or cancer) cell, or a cell of derived from (tissue) of such tumour or cancer; in particular of a solid tumour.

In certain embodiments, a compound of the invention may inhibit SIK3 in the cell involved with the proliferative disorder (eg the tumour cell). In particular of such embodiments, the compound may inhibit SIK3 in such cell preferentially to inhibiting SIK1 and/or SIK2 in such cell; and/or may inhibit SIK3 in such cell preferentially to inhibiting SIK1 and/or SIK2 and/or SIK3 in one or more types of immune cells. For example, a compound of the invention may inhibit SIK3 in the cell involved with the proliferative disorder (eg the tumour cell) preferentially to inhibiting SIK1 and/or SIK2 and/or SIK3 in macrophages and/or dendritic cells (in particular, those capable of or producing IL-10).

A compound (or pharmaceutical composition) of the invention (or a compound used in the invention) may be administered to the subject, in particular in an amount (such as a dose) that is effective to, inhibit SIK3 and/or that is effective to sensitise the cells involved with the proliferative disorder to the cell-mediated immune response. Suitable amounts, formulations and means for such administration are described elsewhere herein.

In particular embodiments, a compound (or pharmaceutical composition) of the invention (or a compound used in the invention) is administered in an amount (such as a therapeutically effective amount) that is effective to reduce activity of SIK3, preferably of SIK3 in (of) the cells involved with the proliferative disorder. In such embodiments, a “therapeutically effective amount” of the compound (or pharmaceutical composition) can be an amount that is capable to reduce the activity of the SIK3 to an applicable level, but that does not lead to significant (eg intolerable) side effects or over-dosage in respect of other activities of the compound (or pharmaceutical composition).

Preferably, the activity of SIK3 is effectively inhibited (reduced), preferably referring to the SIK3 kinase in (of) the cells involved with a proliferative disorder. For example, an “effective” inhibition (or reduction) may include one where the activity is lowered by a degree (or to a level) that has a physiological effect (eg to a therapeutically effective level), such as a reduction by about 10%, 20%, 50%, or more than 50% such as 70% or 90% of activity of the respective kinase. In respect of SIK3, one of such reductions may be desirable to elicit a therapeutic response.

The term “immune cell” is art recognised to describe any cell of an organism involved in the immune system of such organism, in particular of a mammal such as a human. Leukocytes (white blood cells) are immune cells that are involved in the innate immune system, and the cells of the adaptive immune system are special types of leukocytes, known as lymphocytes. B cells and T cells are the major types of lymphocytes and are derived from hematopoietic stem cells in the bone marrow. B cells are involved in the humoral immune response, whereas T cells are involved in cell-mediated immune response. In preferred embodiments of the invention, the immune cell can be a myeloid cell eg a T cell, and in particular (such as when an increase in cell-mediated immune response is required, such as to treat a cancer) the T cell can be a cytotoxic T cell (also known as TC, cytotoxic T lymphocyte, CTL, T-killer cell, cytolytic T cell, CD8+ T-cell or killer T cell). A CTL is a T-cell that is involved in the killing of cancer cells, cells that are infected (particularly with viruses), or cells that are damaged in other ways. Other preferred immune cells for such embodiments can include Tumour-Infiltrating Lymphocytes (TILs). TILs are white blood cells that have left the bloodstream and migrated into a tumour. Typically, TILs are a mix of different types of cells (eg, T cells, B cells, NK cells, macrophages) in variable proportions, T cells being the most abundant cells. TILs can often be found in the stroma and within the tumour itself, and are implicated in killing tumour cells. The presence of lymphocytes in tumours is often associated with better clinical outcomes.

The term “cell-mediated immune response”, as used herein, may include, but is not limited to, a response in a host organism involving, utilising, and/or promoting any one or combinations of T cell maturation, proliferation, activation, migration, infiltration and/or differentiation, and/or the activation/modulation/migration/infiltration of a macrophage, a natural killer cell, a T lymphocyte (or T cell), a helper T lymphocyte, a memory T lymphocyte, a suppressor T lymphocyte, a regulator T lymphocyte, and/or a cytotoxic T lymphocyte (CTL), and/or the production, release, and/or effect of one or more cell-secretable or cell-secreted factor such as a cytokine or autocoid (in particular a pro-inflammatory cytokine such as TNF), and/or one or more components of any of such processes (such as a cytokine or autocoid, particular a pro-inflammatory cytokine such as TNF). The term “cell-mediated immune response,” as used herein, may include a cellular response involving a genetically engineered, in-vitro cultured, autologous, heterologous, modified, and/or transferred T lymphocyte, or it may include a cell-secretable or cell-secreted factor (such as a cytokine or autocoid, in particular a pro-inflammatory cytokine such as TNF) produced by genetic engineering. A cell-mediated immune response is preferably not a humoral immune response, such as an immune response involving the release of antibodies. In certain embodiments, in particular when the proliferative disorder is a cancer or tumour, the cell-mediated immune response is an anti-tumour cell-mediated immune response. For example, one that leads to a reduction in tumour (cell) growth, such as a cytotoxic cell-mediated immune response (such as a cytotoxic T cell and/or TNF exposure) that kills cells of the cancer or tumour.

In certain embodiments, the cell-mediated immune response may be mediated by a cell, such as an immune cell, capable of secreting (eg secreting) pro-inflammatory cytokine, such as one selected from the group consisting of: interleukin-1 (IL-1), IL-8 and IL-12, tumour necrosis factor (TNF), interferon gamma (IFN-gamma), and granulocyte-macrophage colony stimulating factor. In particular of such embodiments, the pro-inflammatory cytokine is tumour necrosis factor (TNF) [alpha].

In other embodiments, the cell-mediated immune response may a cell-secretable or cell-secreted factor (such as a cytokine or autocoid), in particular one secretable or secreted by an immune cell. In particular of such embodiments, the cell-mediated immune response is a pro-inflammatory cytokine, in particular tumour necrosis factor (TNF).

The terms “sensitising”, “sensitisation” and “to sensitise” (and the like), as used herein in the context of cell(s) being sensitised to a cell-mediated immune response, will be understood by the person of ordinary skill, and include the meaning that such cells can exhibit an increased susceptibility to one or more effect (eg a treatment effect) that the cell-mediated immune response may have on such cells. In particular, cells that are so sensitised may, when in the presence of (eg exposed to) a cell-mediated immune response, be killed more easily (such as more rapidly, a greater proportion of cells dying or being killed and/or upon a lower amount or exposure of the cell-mediated immune response) than analogous cells that have not been so “sensitised”. For example, cell(s) so sensitised may be induced into cell-death (eg apoptosis) upon exposure to a lower number of T cells or to a lower concentration of TNF (such as about 10%, 20%, 30% 40%, 50% or more than 50% fewer T cells or lower concentration of TNF). Methods to determine whether such cells have been sensitised (and by which degree) to cell-mediated immune responses are described herein, such as in the examples. Accordingly, in certain embodiments of the present invention, cells involved with the proliferative disorder may be sensitised to cell-death/killing (eg by entry into apoptosis) by a cell-mediated immune response (such as CTL or a proinflammatory cytokine eg TNF).

The terms “tumour necrosis factor” and “TNF” (previously and hence alternatively known as tumour necrosis factor alpha and TNF-alpha) shall, in the context of the herein disclosed invention, be understood to refer to any proteins know under these denotations in the art. In particular, the term TNF encompasses endogenous TNF of any organism where such is present, and preferably of animals or mammals, such as humans. By means of example and not limitation, human TNF may encompass endogenous proteins as disclosed in inter alia Pennica et al. 1984 (Nature 312: 724-9) and in the UniProtKB/Swiss-Prot database with the entry No P01375 (for example, entry version 224 of 15 Mar. 2017), as well as any sequence variants thereof due to normal sequence polymorphism between and within human populations. By means of further non-limiting examples, the term may encompass endogenous TNF proteins as annotated in the UniProtKB/Swiss-Prot database for bovine (Q06599), dog (P51742), goat (P13296), guinea pig (P51435), cat (P19101), horse (P29553), mouse (P06804), chimp (Q8HZD9), pig (P23563), rabbit (P04924), rat (P16599) and others, as well as any sequence variants thereof due to sequence polymorphism between and within populations of each respective species. Further, the term TNF particularly encompasses the soluble, secreted cytokine form of TNF, including monomeric as well as, preferably, the typically more active trimeric forms thereof (see, e.g., Smith & Baglioni 1987. J Biol Chem 262: 6951-4). The primary amino acid sequences of soluble forms of endogenous TNF are indicated in the above mentioned UniProtKB/Swiss-Prot database entries for the respective exemplified organisms. In addition, the term TNF may also encompass membrane-bound forms of TNF expressed on the surface of some cell types (see, e.g., Kriegler et al. 1988. Cell 53: 45-53). Further, the term TNF may also encompass synthetic or recombinant proteins whose primary amino acid sequence is identical or substantially identical (“substantially identical”, as used throughout this specification, generally refers to ?80%, e.g., ?85%, preferably 90%, more preferably 95%, even more preferably 98% or 99% sequence identity) to the sequence of an endogenous TNF, as determined using, e.g., the “Blast 2 sequences” algorithm described by Tatusova & Madden 1999 (FEMS Microbiol Lett 174: 247-250), and which (preferably) retain biological activity identical or substantially identical to the respective endogenous TNF, as determined using, e.g., the cytotoxicity tests described by Flick & Gifford 1984 (J Immunol Methods 68: 167-75). As will appear from the context of aspects and embodiments of the present invention, the term TNF may, in particular, refer herein to endogenous TNF, soluble and/or membrane bound, preferably soluble, produced by cells, tissues, organs or organisms, preferably human. Nevertheless, also envisioned by the term “TNF” are exogenous forms of tumour necrosis factor, in particular those produced by recombinant technologies and, in certain embodiments, may be administered to subjects, or exposed to or contacted with cells in various aspects and embodiments of the invention. In certain of such embodiments, the TNF may be a recombinant TNF used as a therapeutic, such as tasonermin (BEROMUN).

In certain embodiments, the cell-mediated immune response can be mediated by a pro-inflammatory cytokine-secreting cell, such as a lymphocyte (eg a T cell), in particular a cytotoxic T lymphocyte (CTL).

In particular embodiments, the cell-mediated immune response may induce killing (eg cell-death, such via apoptosis) of cells involved with the proliferative disorder. For example, the treatment (method) may comprise (eg may involve) that (or be mediated by) the cell-mediated immune response induces such killing of cells involved with the proliferative disorder.

The cells involved with the proliferative disorder may be killed (eg induced into cell death) by one or more cytotoxic processes, in particular those that are endogenous to such cell such as programmed cell death (PCD). Cell death processes may include, but are not limited to, necrosis (in particular necroptosis), apoptosis, anoikis, autophagy, ferroptosis, mitotic catastrophe and activation-induced cell death. In certain preferred embodiments, the cells involved with the proliferative disorder (eg the tumour cells) are induced into apoptosis by the cell-mediated immune response (eg by TNF). In a further embodiment, a compound (or pharmaceutical composition) of the invention is administered to not kill such cells in the absence of the cell-mediated immune response (eg in the absence of TNF). In particular of such further embodiments, the compound (or pharmaceutical composition) may be administered in an amount (eg in a dose) that is not effective to kill such cells in the absence of the cell-mediated immune response. The examples herein, describe various assays by which an amount of a compound (or pharmaceutical composition) of the invention may be determined that is effective to kill such cells only, or preferentially, in the presence of the cell-mediated immune response.

In other particular embodiments, the cell-mediated immune response may involve at least one immune cell effector molecule, in particular an effector molecule that is secretable or secreted by an immune cell. In particular of such embodiments, the effector molecule can be a pro-inflammatory cytokine, preferably tumour necrosis factor (TNF).

In certain embodiments, the effector molecule is not a cell effector molecule selected from Fas ligand (FasL or CD95L) and TNF-related apoptosis-inducing ligand (TRAIL, CD253 or TNFSF10).

In particular embodiments of the invention, a compound (or pharmaceutical composition) of the invention may be administered to the subject (eg in an amount or dose effective) with the intent to (or so as to) (effectively) sensitise cells involved with the proliferative disorder to killing induced by TNF. For example, the compound (or pharmaceutical composition) may be administered in a therapeutically effective amount, such as an amount effective to sensitise the cells involved with the proliferative disorder to killing (cell-death) induced by TNF.

For example, a compound (or pharmaceutical composition) of the invention may be administered to the subject (for example, in an amount or dose effective) to induce apoptosis of such cells mediated by TNF, such as when such cells are in the presence of or contacted with TNF. In further embodiments, the a compound (or pharmaceutical composition) of the invention may be administered to the subject (eg in an amount or dose effective) to induce a reduced amount of cytotoxicity (eg apoptosis)—such as to not induce killing (eg apoptosis) of such cells—in the absence of TNF; for example the compound (or pharmaceutical composition) may be administered in an amount or dose that is—not as effective in cytotoxicity (eg apoptosis)—such as being not effective to induce such killing—in the absence of TNF.

TNF can induce pro-apoptotic processes via binding to and/or signalling via tumour necrosis factor receptor 1 (TNFR1) and or tumour necrosis factor receptor 2 (TNFR2). Accordingly, in certain embodiments a compound (or pharmaceutical composition) of the invention may be administered to the subject (eg in an amount or dose effective) to (effectively) sensitise cells involved with the proliferative disorder to apoptosis mediated by tumour necrosis factor receptor 1 (TNFR1) signalling and/or tumour necrosis factor receptor 2 (TNFR2) signalling. Preferably, the compound (or pharmaceutical composition) can be administered to the subject (eg in an amount or dose effective) to (effectively) sensitise cells involved with the proliferative disorder to apoptosis mediated thereby in particular mediated by TNFR1. For example, the compound (or pharmaceutical composition) may be administered in a therapeutically effective amount that is effective to mediate TNFR1- and/or TNFR2-signalling, and/or apoptosis mediated thereby.

For example, in certain embodiments, a compound (or pharmaceutical composition) of the invention may be administered (eg in an amount or dose effective) to induce apoptosis of such cells by TNFR1 and/or TNFR2 signalling, such as upon active TNFR1 signalling. In particular of such embodiments, the compound (or pharmaceutical composition) may be administered to the subject (eg in an amount or dose, such as a therapeutically effective amount) to (effectively) induce a reduced amount of cytotoxicity (eg apoptosis)—such as to not induce apoptosis of such cells —in the absence of TNFR1 and/or TNFR2 signalling, such as in the absence of active TNFR1 signalling. For example, the compound (or pharmaceutical composition) may be administered in an amount or does that is not as effective in cytotoxicity (eg apoptosis)—such as being not effective to induce such apoptosis—in the absence of such signalling.

Therefore, in certain embodiments, a compound (or pharmaceutical composition) of the invention may be administered to the subject (eg in an amount or dose) to induce a reduced amount of cytotoxicity (eg apoptosis)—such as to not be cytotoxic—to cells involved with the proliferative disorder in the absence of the cell-mediated immune response.

In particular embodiments, a compound (or pharmaceutical composition) of the invention may be continued to be administered to the subject even if the tumour of the subject is increased in size during treatment. Without being bound to theory, even if an increase in tumour size is observed during such treatment, this may indicate an (enhanced) immune reaction against cells of the tumour (eg, the cells have become sensitised to the cell-mediated immune response; and the tumour is increasing in size because of such immune response), and hence the administration of the compound (or pharmaceutical composition) can, in such embodiments, continued to be administered so as to maintain such sensitivity and associated (enhanced) immune reaction.

As described in PCT/EP2018/060172 (WO 2018/193084), the inhibition of SIK3 is associated with a number of key biological processes or phenotypes, including those surprisingly involved in the control and/or triggering of cytotoxic process innate to cells, such as apoptosis. For example, tumour cells can be sensitised to the apoptotic/cytotoxic effects of TNF by the inhibition of SIK3, acting through pathways and components thereof including liver kinase B1 (LKB1, STK11 or NY-REN-19), histone deacetylase 4 (HDAC4), nuclear factor kappa-light-chain-enhancer of activated B cells (NF-kappaB), and pro-apoptotic genes regulated by NF-kappaB such as Caspase 8 and Caspase 9. Also c-Jun N-terminal kinase (JNK) is a signalling component associated with sensitisation to the apoptotic/cytotoxic effects of TNF by the inhibition of SIK3.

The term “associated with”, in the context of this embodiment (and other embodiments, where applicable) can mean that two components, variables, effects or phenotypes are interrelated with each other, and/or that they are related to (eg correlated to) each other, and/or that there is a causative link between a first and a second component, variable, effect or phenotype (such as the second is in response to the first, the second is a consequence of the first, or the second is caused by the first).

Accordingly, in one such embodiment, administration of a compound (or pharmaceutical composition) of the invention can associate with impairment of NF-kappaB activity (eg, by an enhancement or increase in translocation of NF-kappaB out of the nucleus) in cells involved with the proliferative disorder.

In particular of such embodiments, such impairment of NF-kappaB activity (eg, by an enhancement or translocation of NF-kappaB out of the nucleus) may be associated with (activated) TNF- and/or TNFR1-mediated signalling (or TNFR2-mediated signalling) in such cells.

In certain embodiments, a compound (or pharmaceutical composition) of the invention may be administered to the subject (eg in an amount or dose effective) to impair or inhibit NF-kappaB activity in the cells involved with the proliferative disorder, for example to enhance or increase translocation of NF-kappaB out of the nucleus of such cells. For example, the compound (or pharmaceutical composition) may be administered to the subject in a (eg, therapeutically effective) amount being effective to (effectively) impair NF-kappaB activity in cells involved with the proliferative disorder, in particular in an amount effective to (effectively) enhance or increase translocation of NF-kappaB out of the nucleus of the cells involved with the proliferative disorder.

In alternative or further embodiments, administration of a compound (or pharmaceutical composition) of the invention may be associated with an increase in (eg, the compound (or pharmaceutical composition) is administered, such as in an amount or dose effective, to increase) activity of class II (eg IIa) HDACs, eg HDAC4, in the cells involved with the proliferative disorder, for example its translocation or localisation to or its activity in the nucleus of such cells; and in particular upon TNF- and/or TNFR1-mediated signalling (or TNFR2-mediated signalling) in such cells.

In other alternative or further embodiments, administration of a compound (or pharmaceutical composition) of the invention may be associated with de-acylation of nuclear NF-kappaB (eg de-acylation at its p65 subunit) and/or decreased transactivation of one or more anti-apoptotic factors, in particular upon TNF- and/or TNFR1-mediated signalling (or TNFR2-mediated signalling) in the cells involved with the proliferative disorder. For example, the compound (or pharmaceutical composition) may be administered (such as in an amount or dose effective) to cause de-acylation of nuclear NF-kappaB (eg at its p65 subunit) and/or decreased transactivation of one or more anti-apoptotic factors.

In another alternative or further embodiment, administration of a compound (or pharmaceutical composition) of the invention may be associated with an increase in (eg the compound (or pharmaceutical composition) is administered, such as in an amount or dose effective, to increase) cleavage of Caspase 8 and/or Caspase 9 in the cells involved with the proliferative disorder, in particular upon TNF- and/orTNFR1-mediated (orTNFR2-mediated signalling) signalling in such cells.

In yet other alternative or further embodiments, administration of a compound (or pharmaceutical composition) of the invention may be associated with a reduction in the transcription of one or more anti-apoptotic factors, in particular upon TNF- and/or TNFR1-mediated signalling (or TNFR2-mediated signalling) in the cells involved with the proliferative disorder, for example the reduction of the transcription of one or more NF-kappaB target genes in such cells. In particular, the compound (or pharmaceutical composition) may be administered (eg in an amount dose effective) to reduce the transcription of one or more such anti-apoptotic factors, in particular upon TNF- and/or TNFR1-mediated signalling (or TNFR2-mediated signalling) in the cells involved with the proliferative disorder.

In one embodiment the administration of a compound (or pharmaceutical composition) of the invention may be associated with an increase in (eg the compound (or pharmaceutical composition) is administered, such as in an amount or dose effective, to increase) JNK activation (such as by phosphorylation) in the cells involved with the proliferative disorder, in particular upon TNF- and/or TNFR1-mediated signalling (or TNFR2-mediated signalling) in such cells.

In another embodiment, administration of a compound (or pharmaceutical composition) of the invention may not be associated with a significant change in CREB-pathways signalling and/or a significant change gene expression mediated by CREB and/or CREB-regulation.

In a particular embodiment, the TNF- (TNFR2-) and/or TNFR1-mediated signalling in the cells involved with the proliferative disorder may be associated with increased levels of pLKB1 in such cells.

As will now be apparent to the person of ordinary skill given knowledge of the present invention, the treatment aspects of the invention may further comprise a step of administering one or more other moieties that appropriately modify the expression, activity, function or stability of one or more these other pathway components described above, so as to additively or synergistically contribute to the treatment effect. For example, in one such embodiment, a treatment aspect of the invention may further comprise a step of administering an inhibitor of LKB1. In another of such embodiments, a treatment aspect of the invention may further comprise a step of administering a compound of the invention that promotes, enhances or increases one or more class II (eg IIa) HDACs (histone deacetylases), such as HDAC4, in the nucleus of the cells involved with the proliferative disorder. In yet another of such embodiments, a treatment aspect of the invention may further comprise a step of administering an inhibitor of NF-kappaB (activation). The invention also envisions that combinations of two or more such other moieties may be used in a treatment together with a compound (or pharmaceutical composition) of the invention and/or using other (eg anti-cancer) therapeutically active agents (such as an additional therapeutic agent as described elsewhere herein) together with the compound (or pharmaceutical composition).

In a further aspect, and as may be further described, defined, claimed or otherwise disclosed herein, the invention relates to a method for the sensitisation of cells involved with a proliferative disorder to a cell-mediated immune response, the method comprising exposing (eg contacting) the cells involved with a proliferative disorder to a compound (or pharmaceutical composition) of the invention. Such a method may, typically, be practiced as an in-vitro and/or ex-vivo method.

In a particular embodiment, the cell-mediated immune response comprises killing the cells involved with a proliferative disorder, such as where said killing involves (eg, is mediated, is or supported by) TNF, TNFR2- and/or TNFR1-mediated signalling. For example, the killing of such cells may involve apoptosis of such cells induced by TNF, TNFR2- and/or TNFR1-mediated signalling. Within this and the other applicable embodiments of the various aspects of the invention, TNFR2- and/or TNFR1-mediated signalling may be triggered (eg activated) by any appropriate triggering molecule, such as TNF, a variant of TNF and or a TNFR2 or TNFR1 agonist; in particular by exposing (eg by contacting) the cells associated with the proliferative disorder to the triggering molecule (eg TNF, TNF variant or TNFR1 agonist). Such exposure can lead to the triggering molecule (eg TNF, TNF variant or TNFR1 agonist) binding to TNFR2 and/or TNFR1 and, in particular the triggering (eg activation) of TNFR1 signalling.

In a yet further aspect, and as may be further described, defined, claimed or otherwise disclosed herein, the invention relates to a method for the killing of cells involved with a proliferative disorder, the method comprising exposing (eg contacting) the cell involved with the proliferative disorder to: (i) TNF, a TNF variant and/or an agonist of TNFR1- or TNFR2-signalling (preferably, TNFR1-signalling); and exposing (eg contacting) the cells involved with the proliferative disorder to (ii) a compound (or pharmaceutical composition) of the invention. As will be appreciated by the person or ordinary skill, such a method may, typically, be practiced as an in-vitro and/or ex-vivo method.

In a related aspect, the invention relates to a compound (or pharmaceutical composition) of the invention for use in the treatment of a proliferative disease involving the killing of a cell involved with the proliferative disorder, the treatment comprising exposing such cell to: (i) TNF, a TNF variant and/or a TNFR1 or TNFR2 agonist; and (ii) a compound (or pharmaceutical composition) of the invention.

In particular embodiments of such aspects, the killing of the cell involved with the proliferative disorder is mediated by sensitising such cell to a cell-mediated immune response, in particular by inducing sensitivity to apoptosis of such cell that involves (eg, is mediated, is or supported by) TNF, TNFR2 and/or TNFR1-mediated signalling.

The cell(s) involved with the proliferative disorder may be exposed to the TNF, a TNF variant and/or a TNFR1 or TNFR2 agonist by contacting the cell to such triggering molecule; and/or such cell(s) may be exposed to a compound (or pharmaceutical composition) of the invention by contacting (or introducing into) such cell(s) with a compound (or pharmaceutical composition) of the invention. The amounts (or dose) of (i) TNF, a TNF variant and/or a TNFR1 or TNFR2 agonist; and/or (ii) a compound (or pharmaceutical composition) of the invention are, typically, effective amounts; that is amounts (or doses) that are effective in, for example, sensitising the cell(s) to (such as killing such cell(s) by) apoptosis induced by TNF, TNFR2 and/or TNFR1-mediated signalling. Elsewhere are disclosed suitable amounts of these active agents (or ways to determine them) that may be incorporated in these aspects of the invention; as are further particular characteristics of the compound (or pharmaceutical composition) of the invention. Accordingly, in certain embodiments: (i) TNF, a TNF variant and/or a TNFR1 or TNFR2 agonist; and (ii) a compound (or pharmaceutical composition) of the invention, can be administered to a subject suffering from the proliferative disorder (eg, the treatment can comprise the administration of: (i) TNF, a TNF variant and/or a TNFR1 or TNFR2 agonist; and (ii) a compound (or pharmaceutical composition) of the invention, can be administered to the subject).

The cell(s) involved with the proliferative disorder may be one as described elsewhere herein, and in particular such cell(s) may be cancerous or tumour cell. For example, such cell(s) may be one that is of, or derived from, a solid tumour.

In certain embodiments of these aspects, the method is an in vitro (and/or ex-vivo) method. In alternative embodiments of such methods, the cell(s) involved with the proliferative disorder (such as tumour cells) is present in such subject, in particular in a subject in need of treatment thereof.

In further embodiments of the methods of these aspects, the (treatment) effect of such method (eg, on the cell(s) involved with the proliferative disorder) can be mediated by (eg, the treatment may comprise, involve or be mediated by) inhibiting SIK3; in particular, by inhibiting the function and/or activity of SIK3 protein (eg, of phosphorylated SIK3 protein, and/or as described elsewhere herein). In particular, in such embodiments, the SIK3 activity is (eg, effectively) reduced, such as reduced to a therapeutically effective level.

In certain embodiments of such methods, in the absence of (eg such effective amount or dose of) a compound (or pharmaceutical composition) of the invention, the cell(s) involved with the proliferative disorder (such as the tumour cell(s)) are not killed or induced to enter apoptosis (for example, they proliferate) upon TNF, TNFR2- and/or TNFR1-mediated signalling and/or exposure to (eg, the effective amount or dose of) TNF, TNF variant, TNFR2 or TNFR1 agonist.

As described above, in certain embodiments of these methods, a compound (or pharmaceutical composition) of the invention may inhibit SIK3 in (of) the cell(s) involved with the proliferative disorder (eg tumour cells). In particular of such embodiments, the compound (or pharmaceutical composition) may inhibit SIK3 in (of) such cell(s) preferentially to inhibiting SIK1 and/or SIK2 in (of) such cell; and/or may inhibit SIK3 in such cell preferentially to inhibiting SIK1 and/or SIK2 and/or SIK3 in (of) one or more types of immune cells. For example, a compound (or pharmaceutical composition) of the invention may inhibit SIK3 in (of) the cell(s) involved with the proliferative disorder (eg tumour cells) preferentially to inhibiting SIK1 and/or SIK2 and/or SIK3 in (of) macrophages and/or dendritic cells (in particular, those capable of or producing IL-10). In particular embodiments, the (treatment) effect is mediated by (eg, the treatment comprises, involves, is by or is mediated by) inhibition of SIK3 in (of) the cell(s) involved with the proliferative disorder (eg a tumour cell); and in further of such embodiments, the (treatment) effect is not mediated by (or the effect is mediated by not) (eg, the treatment does not comprise, involve or is not mediated by) inhibiting SIK2, in particular SIK2 in/of other cells (such as those involved with the proliferative disorder or immune cells), and/or the (treatment) effect is not mediated by (or the effect is mediated by not) inhibiting SIK1 (eg, the treatment does not comprise, involve or is not mediated by inhibiting SIK1), in particular SIK1 in/of other cells (such as those involved with the proliferative disorder or immune cells).

Accordingly, in one embodiment, the SIK3 of (eg, in) the cell(s) involved with the proliferative disorder is inhibited (eg, by a compound or pharmaceutical composition of the invention). In another (or further) embodiment, another kinase (eg SIK2, in particular SIK2) of (eg in) immune cells—such as CTLs—is inhibited to a lesser extent than SIK3 (eg in the cell(s) involved with the proliferative disorder). In yet another (or further) embodiment SIK1, in particular SIK1 of (eg in) immune cells—such as CTLs—is inhibited to a lesser extent than such SIK3.

In certain of such embodiments, one or more of the kinases selected from the list consisting of: SIK2, SIK1, ABL1, SRC, BCR-ABL, LCK, LYN, YES, FYN, KIT and FLT3 (such as SIK2 and/or LCK) is inhibited (eg, by a compound or pharmaceutical composition of the invention) to a lesser extent than one or more of the kinases selected from the list consisting of:) SIK3, SIK2, ABL/BCR-ABL, KIT, NEK2, BRAF. CSF1R, HCK, TEC-family kinases (eg BTK, TXK or ITK), AXL, BLK, TYRO3, MERTK, ZAP70, SYK, EGFR, and/or BRK, (in particular, SIK3 or BRK).

A given kinase (such as SIK1 or SIK2) may be inhibited to a “lesser extent” than another kinase (such as SIK3) if, for example, the other kinase (such as SIK3) is inhibited by an amount greater than about 2 fold more than the given kinase, such as by an amount greater than about 5, 10, 20, 50, 75 or 100-fold more than the given kinase. In particular, the other kinase (such as SIK3) may be inhibited by an amount between about 5 and 20 fold, 20 and 50 or 50 and 100 fold more than the given kinase. For example, the SIK3 (ie, the other kinase) may be inhibited between about 20 and 50 fold more than SIK1 and/or SIK2 (ie, a given kinase). By way of example, a compound (or pharmaceutical composition) of the invention may inhibit the other kinase (eg SIK3) by 80% (ie, to have only 20% of its uninhibited activity) but inhibit the given kinase (eg SIK1) by only 4% and SIK2 by only 8%. Accordingly, the other kinase (eg SIK3) is inhibited about 20-fold more than the given kinase (eg SIK1) and 10-fold more than another given kinase (eg SIK2). In particular embodiments, the other kinase (eg SIK3) may be inhibited to about the same extent as eg SIK1 (eg between about 2 to 53 fold of each other), and eg SIK2 is inhibited to a lesser extent that either (or both) of eg SIK3 and SIK1: For example, in such embodiments, eg SIK3 and SIK1 are inhibited by between about a 20 and 50 fold more than eg SIK2 (eg in immune cells) is inhibited.

The expression “inhibited to a “lesser extent” can be also applied by analogy, in the applicable embodiments as the context suggests, when comparing the relative inhibitory properties of one compound (eg, a compound of the invention) to another compound (eg, prior art compound PY1).

In certain of such embodiments, one or more of the kinases selected from the list consisting of: BLK, SIK3 and TEC, is inhibited by one or more of compounds of the invention (eg by compound AA1) to a lesser extent than by PY1.

In certain of such embodiments, one or more of the kinases selected from the list consisting of: BRAF, NEK2, PRK2, PKC, and in particular KIT, RIPK2, ABL2 and PDGF-alpha, is inhibited by PY1 to a lesser extent than one or more of compounds of the invention (eg by compound AA11); and/or one or more of the kinases selected from the list consisting of: BMX, TEC and in particular BTK, is inhibited by one or more of compounds of the invention (eg by compound AA11) to a lesser extent than by PY1.

In certain of such embodiments, one or more of the kinases selected from the list consisting of: TAOK2, SYK, TYRO3, ACVR2B, MEKK2, AXL, ITK, MAP3K11, TRKA, MERTK, ZAP70, and MEKK2 (or in particular CSF1R, HCK, TXK, YES, LCK, SRC, EPHA1 or FGR), is inhibited by PY1 to a lesser extent than one or more of compounds of the invention (eg by compound AA3); and/or one or more of the kinases selected from the list consisting of: SRMS, NLK, RIPK5, LTK and ALK, is inhibited by one or more of compounds of the invention (eg by compound AA3) to a lesser extent than by PY1.

In certain of such embodiments, one or more of the kinases selected from the list consisting of: FYN, BTK, EPHB2, LCK and CSK (in particular LCK) is inhibited by one or more of compounds of the invention (eg by compound AA5) to a lesser extent than by PY1; and/or such compound of the invention (eg AA5) inhibits one or more kinase selected from the list consisting of: TXK, ERBB4, EPHB1, FRK, BRK, EPHA4, ACK1, EGFR, EPHA1 and SIK1 (or in particular TXK, BRK or CSF1R).

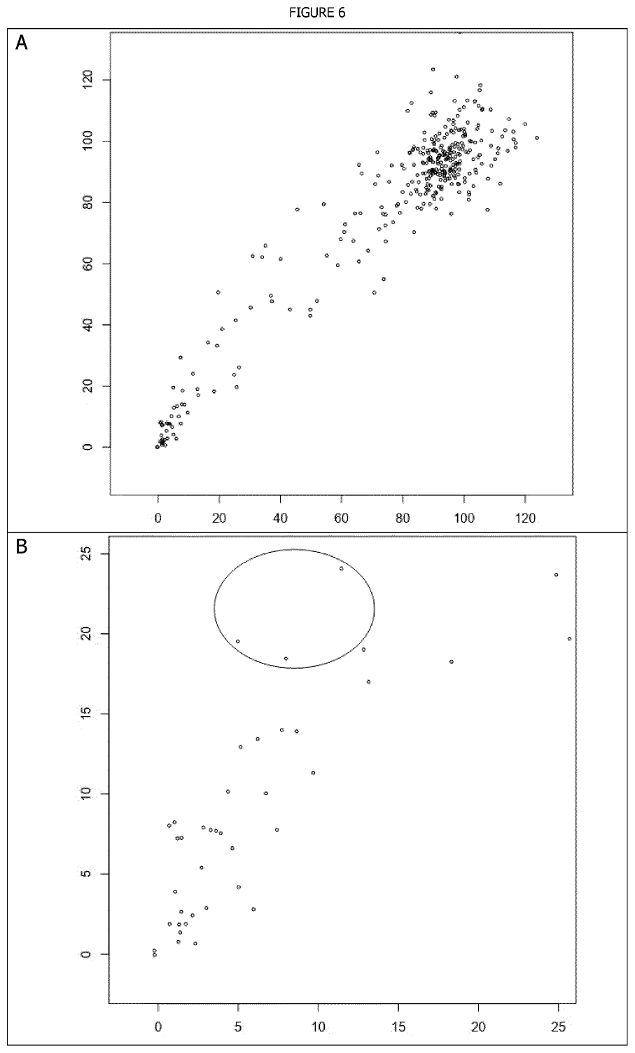

The compounds of the invention (in particular those of formula (I), (Ia), (II), (III), (IV), (V), (Va), (VI), (VII), (VIIa), (VIII), (IX), (X), (XI) or (XII)) and/or compounds used in the invention are shown to be potent inhibitors of one or more kinases (as shown in the Examples, and in particular by FIG. 6 to FIG. 9). In particular, any of (or any combination of) those kinases described in Example 3 having a residual activity of between about 50% and about 25%, or less than about 25% residual activity (and particular, those having a residual activity of less than about 10%), are considered, in certain embodiments to be “key-kinases” that are inhibited by the respective compounds of the invention. Mutants of such kinases are also considered therein. As particular examples, the key-kinases include one or more kinases selected from the list consisting of: SIK3, SIK2, SIK1, ABL/BCR-ABL, KIT, NEK2, BRAF. CSF1R, HCK, TEC-family kinases (eg BTK, TXK or ITK), AXL, BLK, TYRO3, MERTK, ZAP70, SYK, EGFR and BRK; and/or selected from the list consisting of: FLT3, LCK, PHA2, EPHA4, ACK1, NEK11, WEE1, WNK2, Aurora-A, Aurora-B and TBK1.

The inventors find that, despite the significant structural differences compared to the prior art compound PY1 (YKL-05-099) the compounds of the invention are potent inhibitors of various disease related kinases such as one selected from the group consisting of: SIK3, SIK2, SIK1, ABL/BCR-ABL, KIT, NEK2, BRAF. CSF1R, HCK, TEC-family kinases (eg BTK, TXK or ITK), AXL, BLK, TYRO3, MERTK, ZAP70, SYK, EGFR and BRK; and/or selected from the group consisting of: FLT3, LCK, PHA2, EPHA4, ACK1, NEK11, WEE1, WNK2, Aurora-A, Aurora-B and TBK1L. Accordingly, in one embodiment a compound of (or for use in a method of) the invention is an inhibitor of one or more of the kinases selected from the group consisting of: SIK3, SIK2, BRAF, NEK2, PRK2 and PKC (or in particular KIT, RIPK2, ABL2 or PDGF-alpha); and/or selected from the group consisting of: SIK3, SIK2, TAOK2, SYK, TYRO3, ACVR2B, MEKK2, AXL, ITK, MAP3K11, TRKA, MERTK, ZAP70, and MEKK2 (or in particular CSF1R, HCK, TXK, YES, LCK, SRC, EPHA1 or FGR); or in particular, selected from the group consisting of: SIK3, CSF1R, HCK, TEK-family, BRK, ABL and KIT. In one particular embodiment, such a compound may be an inhibitor of SIK3 kinase. In one further (or alternative) particular embodiment, such a compound may be an inhibitor of CSF1R kinase, and/or for example is a compound that is capable of depleting (eg depletes) M2-like tumour associated macrophages (TAMs) in an MC38 syngeneic mouse tumour model such as one analogous to that described in Example 9 herein. In another further (or alternative) particular embodiment, such a compound may be an inhibitor of HCK kinase, and/or for example is a compound that is capable of inhibiting the formation of podosomes within TAMs (such as TAMs in an MC38 syngeneic mouse tumour model analogous to one described in Example 9 herein).

As described above, the inventors find that compounds disclosed herein inhibit a different set of kinases and/or each to a different degree compared to other kinase inhibitors

Compounds that inhibit different kinases and/or kinases to different degrees will have different properties in vivo, and can be used for different medical indications, or for the same medical indications but showing different properties in terms of efficacy and side-effects. As will be appreciated, compounds with different specificity to kinases can have surprisingly different properties and applications.

Accordingly, in one embodiment, the treatment comprises (eg, involves, is by or is mediated by) inhibition of one or more of the key-kinases (eg of SIK3, SIK2, SIK1, ABL/BCR-ABL, KIT, NEK2, BRAF. CSF1R, HCK, TEC-family kinases (eg BTK, TXK or ITK), AXL, BLK, TYRO3, MERTK, ZAP70, SYK, EGFR, and/or BRK and/or FLT3, LCK, PHA2, EPHA4, ACK1, NEK11, WEE1, WNK2, Aurora-A, Aurora-B and TBK1). In particular of such embodiments, the treatment comprises (eg, involves, is by or is mediated by) inhibition of such key-kinase(s) more than comprising (eg, involving, is or is mediated by) inhibition of one or more of the other key-kinases (eg SIK3 and/or SIK1 and/or SIK2). For example, the treatment can involve inhibiting SIK3, SIK2, CSF1R, HCK, BTK, TXK or ITK, BRK, ABL and/or KIT.

In a particular (alternative or additional) embodiment, the treatment does not comprise (eg, does not involve, is not or is not mediated by) inhibition of one or more of the key-kinases. In particular of such embodiments, the treatment does not comprise (eg, does not involve, is not or is not mediated by) inhibition of SIK3, and/or the treatment does not comprise (eg, does not involve, is not or is not mediated by) inhibition of SIK1 and/or SIK2.

In further embodiments, the treatment may not comprise (eg, may not involve, is not or is not mediated by) inhibition of one or more of following kinases: FYN, BTK, EPHB2, LCK and/or CSK; and/or SRMS, NLK, RIPK5, LTK and/r ALK; and/or BMX, TEC and in particular BTK.

In one particular (alternative or additional) embodiment, the treatment comprises (eg, involves, is by or is mediated by) inhibition of SIK3.

In another particular (alternative or additional) embodiment, the treatment comprises (eg, involves, is by or is mediated by) inhibition of CSF1R.

In yet another particular (alternative or additional) embodiment, the treatment comprises (eg, involves, is by or is mediated by) inhibition of HCK.

In yet another particular (alternative or additional) embodiment, the treatment comprises (eg, involves, is by or is mediated by) inhibition of BTK.

In yet another particular (alternative or additional) embodiment, the treatment comprises (eg, involves, is by or is mediated by) inhibition of ITK.

In yet another particular (alternative or additional) embodiment, the treatment comprises (eg, involves, is by or is mediated by) inhibition of TXK.

In yet another particular (alternative or additional) embodiment, the treatment comprises (eg, involves, is by or is mediated by) inhibition of BLK.

In yet another particular (alternative or additional) embodiment, the treatment comprises (eg, involves, is by or is mediated by) inhibition of TYRO3.

In yet another particular (alternative or additional) embodiment, the treatment comprises (eg, involves, is by or is mediated by) inhibition of AXL.

In yet another particular (alternative or additional) embodiment, the treatment comprises (eg, involves, is by or is mediated by) inhibition of MERTK.

In yet another particular (alternative or additional) embodiment, the treatment comprises (eg, involves, is by or is mediated by) inhibition of ZAP70.

In yet another particular (alternative or additional) embodiment, the treatment comprises (eg, involves, is by or is mediated by) inhibition of SYK.

In yet another particular (alternative or additional) embodiment, the treatment comprises (eg, involves, is by or is mediated by) inhibition of BRK.

In yet another particular (alternative or additional) embodiment, the treatment comprises (eg, involves, is by or is mediated by) inhibition of KIT.

In one further particular (alternative or additional) embodiment, the treatment comprises (eg, involves, is by or is mediated by) inhibition of FLT3.

In yet another particular (alternative or additional) embodiment, the treatment comprises (eg, involves, is by or is mediated by) inhibition of NEK11. P In one further embodiment, the treatment comprises (eg, involves, is by or is mediated by) inhibition of a mutant of either ABL1 or KIT kinase; such as the inhibition of BCR-ABL, or another mutant of ABL1, such as one selected from the list consisting of: G250E, Q252H, Y253F, E255K, F317I, M351T and H396P.

As described elsewhere herein, in one (alternative or additional) embodiment, compounds of the invention sensitise (eg, the treatment comprises, involves, is by or is mediated by sensitisation of) cells involved with a proliferative disorder to a cell-mediated immune response (such as TNF). However, in an alternative (alternative or additional) embodiment, the compounds do not sensitise (eg, the treatment does not comprise, involve, is not by or is not mediated by sensitisation of) cells involved with a proliferative disorder to a cell-mediated immune response (such as TNF).

In contrast to other studies using kinase (eg SIK) inhibitors, treatment with a compound (or pharmaceutical composition) of the invention in accordance with the present invention, in certain embodiments, may not be associated with an (effective) increase in the production of one or more anti-inflammatory cytokines (for example the anti-inflammatory cytokine may be one selected from the list consisting of: IL-1ra, IL-4, IL-10, IL-11, IL-13 and TGF-beta), and in particular may not be associated with an (effective) increase in the production of IL-10. Correspondingly, in other or further embodiments, treatment with a compound (or pharmaceutical composition) of the invention in accordance with the present invention may not be associated with an (effective) decrease in the production of one or more pro-inflammatory cytokines; for example, one selected from the list consisting of: IL-1-beta, IL-6, IL-12 and TNF, IFN-gamma and granulocyte-macrophage colony stimulating factor, and in particular embodiments may not be associated with an (effective) decrease in the production of TNF. Accordingly, in certain embodiments, a compound (or pharmaceutical composition) of the invention may be administered to a subject in: (i) a (therapeutically effective) amount NOT effective to (effectively) increase the production of one or more (eg such) anti-inflammatory cytokines; and/or (ii) in a (therapeutically effective) amount NOT effective to (effectively) decrease the production of one or more (eg such) pro-inflammatory cytokines.

Certain cells involved with the proliferative disorder (eg tumour cells) may, in certain embodiments, be expected to be more susceptible to the sensitising effects of a compound (or pharmaceutical composition) of the invention in the various aspects of the invention. For example, such cells may be those that exhibit (eg are subject to) activation of TNFR2- and/or TNFR1-signalling, in particular an activated TNFR1. In certain embodiments, such cells are those that express TNFR2 and/or TNFR1, in particular tumour cells that express TNFR1. Accordingly, in certain embodiments, such cells are distinguished or characterised by activated TNFR1- and/or TNFR2-signalling (or the subject is distinguished or characterised by having cells involved with the proliferative disorder—eg tumour cells—that are so distinguished or characterised). The person of ordinary skill will know techniques for determining the status of TNFR1- and/or TNFR2-activation in such cells (such as of the subject). For example, by detecting or monitoring one or more down-stream protein in the TNFR1- and/or TNFR2-signalling pathways. Such proteins are described elsewhere herein, and include NF-kappaB and/or HDAC4.

In one related aspect, the invention relates to a method for the treatment of a proliferative disorder (such as a tumour) in a subject, the (treatment) method comprising administering a compound (or pharmaceutical composition) of the invention to the subject, by inhibiting a kinase/key-kinase (eg SIK3), wherein cells involved with the proliferative disorder are characterised by (eg exhibit or are subject to) activated TNFR2- and/or TNFR1-signalling (eg activated TNFR1 signalling). In another related aspect, the invention relates to a compound (or pharmaceutical composition) of the invention for use in the treatment of a proliferative disorder, wherein cells involved with the proliferative disorder are distinguished or characterised by (eg exhibit or are subject to) activated TNFR2- and/or TNFR1-signalling (eg activated TNFR1 signalling).

In certain embodiments of the various aspects of the invention, cells involved with the proliferative disorder are those exposed to an appropriate triggering or activating molecule, such as TNF, a variant of TNF and or an agonist of TNFR2- or TNFR1-signalling (preferably, an agonist of TNFR1-signalling), in particular are exposed to an effective amount of such triggering or activating molecule.

In particular embodiments, when the triggering or activating molecule is TNF, it is human TNF. In certain of such embodiments, the TNF is recombinant human TNF (rHuTNF). However, in other embodiments the TNF is endogenous TNF, such as that is produced by or otherwise present in the subject (eg the human patient).

Studies have shown that plasma TNF levels are elevated in numerous types of cancers, including in ovarian cancer (Dobrzycka et al 2009, Eur Cytokine Netw 20:131), and that for example, the upper normal limit of total TNF in healthy subjects is 1.8 pg/mL, as measured using a Quantikine human TNF-alpha Immunoassay PDTA00C. In other cancers and assays (eg, TNF-alpha-EASIA Kit, DIAsource), the TNF plasma levels of oesophageal cancer patients and the control group were 12.35 f 9.69 and 4.62 f 3.06 pg/mL, respectively (Aydin et al 2012, Turk J Med Sci 42:762). Accordingly, in other embodiments the cells involved with the proliferative disorder are (for example a tumour is) one present in a subject having a plasma concentration of TNF greater than about 1.5, 2.5 or 4 pg/mL, such as greater than about 5 pg/mL, and in particular greater than about 10 pg/mL (for example, as measured by a Quantikine human TNF-alpha Immunoassay PDTA00C or a TNF-alpha-ELISA Kit, DIAsource).

Accordingly, in one particular embodiment, the subject involved in the treatment methods of the invention may have (that is, such a subject can be distinguished by, such as distinguished as one suitable for the therapeutic methods of the present invention, by showing, possessing or displaying) a plasma concentration of TNF greater than about 2 pg/mL or greater than about 5 pg/mL (eg, the cells involved with the proliferative disorder are one present in a subject having a plasma concentration of TNF greater than about 2 pg/mL or 5 pg/mL).

Indeed, in those embodiments where the proliferative disorder is a tumour, then the intratumoural concentration of TNF may be a characterisation of the tumour, such as when the tumour is a solid tumour and accessible for biopsy (Reissfelder et al 2015, J Clin Inv 125:739). For example, a tumour (such as a solid tumour eg colorectal cancer) can, in some embodiments of the invention, have an intratumoural concentration (eg, within the tumour tissue) of TNF that is greater than about 0.2, 0.5 or 1 pg/mL, such as greater than about 2 pg/mL, and in particular greater than about 5 pg/mL (for example, as measured by a Quantikine human TNF-alpha Immunoassay).

Accordingly, in such embodiments when the proliferative disorder is a tumour (eg a solid tumour), then the solid tumour (eg, within the subject) may have (that is, such a subject can be distinguished by, such as distinguished as one suitable for the therapeutic methods of the present invention, by showing, possessing or displaying) an intratumoural concentration of TNF greater than (about) 0.5 pg/mL or greater than about 1 pg/mL.

Accordingly, in a related aspect, the invention can relate to a method for the treatment of a proliferative disorder (or a compound (or pharmaceutical composition) of the invention for use in such a treatment) in a subject distinguished by having: (i) a plasma concentration of TNF greater than about 2 pg/mL (preferably greater than about 5 pg/mL); and/or (ii) an intratumoural concentration of TNF greater than about 0.5 pg/mL preferably greater than about 1 pg/mL), the treatment method comprising administering a compound (or pharmaceutical composition) of the invention to the subject, wherein the compound (or pharmaceutical composition): (a) inhibits a kinase/key-kinase (eg SIK3) in cells involved with the proliferative disorder; and/or (b) sensitises cells in the subject involved with the proliferative disorder to a cell-mediated immune response.

In particular of such embodiments, the amount (or dose) of a compound (or pharmaceutical composition) of the invention that is exposed to cells involved with the proliferative disorder, or that is administered to the subject, is related to (eg correlated to) the plasma or intratumoural concentration of TNF, wherein a greater amount (or dose) of the compound (or pharmaceutical composition) is exposed to such cells (or administered to such subject) in those cases of a greater plasma or intratumoural concentration of TNF.

In other or further embodiments, the tumour may be present in a subject having tumour-reactive T-cells in peripheral blood or bone marrow, for example as may be determined by IFN-gamma ELISPOT. In yet other or further embodiments, the tumour shows infiltration by Tregs, CD4+ Tconv and/or CD8+ T cells.

In other embodiments, the cells involved with the proliferative disorder comprises a single nucleotide polymorphism (SNP) in the promoter region of TNF associated with increased expression of TNF and cancer sensitivity, for example with an AA or GA genotype at the −308G/A SNP in the promoter region of TNF; and in alternative embodiments the tumour does not comprise a SNP associated with decreased expression of TNF and reduced cancer risk, such as does not comprise an AA or GA genotype at the −238G/A SNP or a −857T allele, in each case in the promoter region of TNF (Wang and Lin 2008, Acta Pharmacol Sin 28:1275).

The invention hereby provides alternative combination treatment regimens based on the surprising finding of the inventors that inhibition of one or more kinase, such a one or more key-kinases, (eg SIK3) by compounds of the invention can influence the sensitivity of a cell towards the apoptotic/cytotoxic effects of TNF. Accordingly, in a further aspect, and as may be further described, defined, claimed or otherwise disclosed herein, the invention relates to a method for the treatment of a proliferative disorder in a subject, the method comprising exposing (eg contacting) cells involved with the proliferative disorder in the subject to: (i) TNF, a TNF variant and/or an agonist of TNFR2- or TNFR1-signalling; and exposing (eg contacting) the cells involved with the proliferative disorder in the subject to (ii) a compound (or pharmaceutical composition) of the invention. In certain embodiments, step (i) of such method does not comprise exposing (eg contacting) cells involved with the proliferative disorder in the subject to a TNF variant.

In certain embodiments, the proliferative disorder and/or such cells are those of the tumour, and in other embodiments, component (i) is TNF, in particular human TNF (such as rHuTNF); and/or component (i) is an agonist of TNFR1-signalling.

In particular embodiments, the method comprises (eg the treatment comprises, involves, is by or is mediated by) increasing the amount of TNF exposed to the cells involved with the proliferative disorder in the subject.

In certain embodiments of such aspects, the treatment may comprise (eg, involves, is by or is mediated by) increasing TNFR1- and/or TNFR2-signalling in (of) the cells involved with the proliferative disorder in the subject. Accordingly, in a related aspect the invention relates to a method for the treatment of a proliferative disorder in a subject, the method comprising: (i) increasing TNFR1- and/or TNFR2-signalling in (of) the cells involved with the proliferative disorder; and (ii) exposing (eg contacting) the cells involved with the proliferative disorder in the subject to a compound (or pharmaceutical composition) of the invention.

In particular the method can, for example, be effected though the consequence(s) of inhibition of a kinase (eg a key-kinase such as SIK3) (such as inhibition of the function and/or activity of phosphorylated SIK3), in particular in combination with the consequence(s) of activation of TNFR1- and/or TNFR2-signalling, such as upon binding of the TNF, TNF variant and/or TNFR1 agonist to TNFR1 or TNFR2.

Accordingly, the treatment effect can, in certain embodiments, involve, or be mediated (eg, caused) by, inhibiting a kinase (eg a key-kinase, such as SIK3), and/or by sensitising the cells involved with the proliferative disorder to the cytotoxic (eg apoptotic) effects of TNFR1- or TNFR2-signalling. In particular of such embodiments, the kinase/key-kinase activity may be (effectively) reduced, such as to a therapeutically effective level.

As described above, herein are also envisioned embodiments wherein a kinase, such as a key-kinase (eg SIK3) in the tumour cells is inhibited and, optionally, where one or more other kinase/key-kinase (eg SIK2 and/or SIK1) are inhibited to a lesser extent, such as such other kinase (eg SIK2 or SIK1) of immune cells.

Also as described above, herein are also envisioned embodiments wherein the treatment comprises, involves, is by or is mediated by (eg, a compound (or pharmaceutical composition) of the invention is administered in an amount, such as a therapeutically effective amount that is effective to) inhibition of a kinase/key-kinase activity such that it is (eg, effectively) reduced, such as reduced to a therapeutically effective level.

In certain embodiments of such aspect, the subject can be administered a compound (or pharmaceutical composition) of the invention and/or can be administered (the) TNF, an (the) TNF variant or an (the) agonist of TNFR1- or TNFR2-signalling.

In such embodiments, a compound (or pharmaceutical composition) of the invention and the TNF, TNF variant or TNFR1 or TNFR2 agonist can be exposed to (for example administered in) an effective amount (or dose), including in formulations or administrative routes as described elsewhere herein. In particular are envisioned embodiments where the TNF, TNF variant or TNFR1 or TNFR2 agonist is encapsulated as a liposomal or other nanoparticle formulation.

When the TNF, TNF variant or TNFR1 or TNFR2 agonist is exposed/administered and a compound (or pharmaceutical composition) of the invention is exposed/administered, then such combination treatment regimen may comprise embodiments where such exposures/administrations are concomitant. In alternative embodiments such exposures/administrations may be sequential; in particular those embodiments where a compound (or pharmaceutical composition) of the invention is exposed/administered before the TNF, TNF variant or TNFR1 or TNFR2 agonist is exposed/administered. For example a compound (or pharmaceutical composition) of the invention may be sequentially exposed/administered within about 14 days of (eg before) the other component, such as within about 10 days, 7 days, 5 days, 2 days or 1 day of (eg before) the other component; and further including where the compound (or pharmaceutical composition) may be sequentially exposed/administered within about 48 hours, 24 hours, 12 hours, 8 hours, 6 hours, 4 hours, 2 hours, 1 hours, 30 mins, 15 mins or 5 mins of (eg before) the other component.

The TNF or the TNF variant or TNFR1 or TNFR2 agonist may be administered via conventional routes, such as s.c., i.v. or i.m., and on certain embodiments may be administered intratumourally or by isolated limb perfusion (ILP), such as isolated hepatic perfusion (IHP); and/or may be so administered (in particular, rHuTNF may be so administered) at a dose of between about 5 and 500 μg/m²/day. For example, TNF may be administered between about 25 and 250 μg/m²/day, such as between about 50 and 150 μg/m²/day or between about 75 and 100 μg/m²/day; or wherein TNF is administered up to a MTD of about 50 and 75 μg/m²/day when administered s.c. or up to a MTD of about 150 and 200 μg/m²/day when administered i.v. or i.m. Accordingly, in particular of such embodiments, TNF can be administered to the subject at a dose of between about 5 and 500 μg/m²/day, in particular between about 20 and 200 μg/m²/day.

In particular embodiments a variant of TNF, such as a TNF variant having higher anti-tumour activity and lower systemic toxicity that rHuTNF may be exposed/administered. For example, the TNF variant may be one selected from the group consisting of: (i) a −K90R variant of TNF; (ii) a tumour-homing peptide conjugated to TNF; and (iii) a TNF-antibody conjugate.

In those embodiments of the invention involving a TNF variant, it may be a variant form of TNF having higher cytotoxic activity and lower systemic toxicity.

In other embodiments a TNFR1 or TNFR2 agonist, such as the anti-TNFR1 monoclonal antibody htr-9 (Ferrero et al 2001, Am J Physiol Cell Physiol 281:C1173) may be exposed/administered, and in other embodiments lymphotoxin-alpha (Etemadi et al 2013, FEBS J 280:5283) or a variant thereof may be exposed/administered.

In alternative embodiments, cells involved with the proliferative disorder (eg tumour cells) may be exposed to TNF (or increased TNFR1- and/or TNFR2-signalling) through the administration of an agent (eg to a subject harbouring such cell) that can lead to the exposure of such cells to (eg endogenous) TNF, or to another triggering molecule such as a variant of TNF or a TNFR1 or TNFR2 agonist. Such an agent may, for example, be one that is capable of inducing (eg induces) the exposure of such cells to (eg an elevated level of) TNF, in particular an agent that induces the exposure of such cells to TNF levels, such as to an effective amount of (eg endogenous) TNF, for example levels of plasma or intratumoural TNF that are greater than one or those levels described elsewhere herein.

Accordingly, the invention includes those embodiments wherein the subject is administered an agent that is capable of inducing (eg induces) the exposure of the cells involved with the proliferative disorder to (the) TNF, an (the) TNF variant or an (the) agonist of TNFR1- or TNFR2-signalling. The invention also includes those embodiments wherein the subject gets administered an agent that is capable of increasing TNFR1-signalling (and/or TNFR2-signalling) of, and/or increasing the amount of TNF exposed to, cells involved with the proliferative disorder in the subject.

In certain of such embodiments, the agent is a virus, in particular one that has been engineered to produce a triggering molecule being TNF, a TNF variant or the TNFR1 or TNFR2 agonist (especially, a virus engineered to produce human TNF). Further of such embodiments include those where such virus preferentially infects the cell(s) involved with the proliferative disorder (eg tumour cells) and/or preferentially produces the triggering molecule in the context of (eg when it infects) such cells. As will now be apparent, the administration of such a virus can lead to the exposure of the cell(s) involved with the proliferative disorder to such triggering molecule, and in particular to an effective amount of such a triggering molecule such as TNF.

Accordingly, in certain of such methods, the agent may be a virus that is capable of inducing (eg induces) the exposure of the cell(s) involved with the proliferative disorder the TNF, TNF variant or agonist of TNFR1- or TNFR2-signalling.

Such a virus may be any that is suitable for inducing the exposure of the triggering molecule, and in particular may be a recombinant virus; for example, one engineered to infect tumour cells and/or to express TNF (eg after infecting a tumour cell). Examples of virus that may be so engineered include oncolytic viruses (eg, those based on an adenovirus, HSV, vaccinia virus, vesicular stomatitis virus or Newcastle disease virus), such as intratumoural injection of adenovirus vectors to increase plasma levels of pro-inflammatory cytokines and chemokines, including TNF (Bernt et al 2005, Cancer Res 65:4343). In particular of such embodiments, the oncolytic virus may be one based on a DNA virus described in Table 1 of Kaufman et al 2015 (Nature Rev Drug Disc 14:642), one based on an RNA virus described in Table 2 of Kaufman et al 2015, preferably, is an oncolytic virus described in Table 3 of Kaufman et al 2015 as being in clinical trials.

In other of such embodiments, the agent that is administered (and that consequentially leads to exposure of the cells involved with the proliferative disorder to a triggering molecule being TNF, a TNF variant or a TNFR1 or TNFR2 agonist) is an immune cell. In certain of such embodiments, the immune cell may not be an IL10-producing macrophage, for example the immune cells can be a pro-inflammatory immune cell. In particular of such embodiments, the immune cell that is administered may be a lymphoid cell, eg a T cell or a natural killer (NK) cell, for example such a cell that produces TNF.

When administered as an agent in such embodiments of the invention, the immune cell may be administered via adoptive cell transfer (ACT); meaning the transfer of the immune cell into the subject (eg, by infusion or other delivery techniques). Such process is, typically, conducted with the goal of improving immune functionality and characteristics in the subject, and while conventionally the transferred immune cells will have originated from the same subject, they may alternatively have been derived from another (suitable) individual.

When used in this embodiment of the invention, the immune cells may be T cells extracted from the subject, genetically modified and cultured in vitro and returned to the same subject, such as in a therapeutic method of the invention. Such genetic modification can include those that enhance the specificity or targeting of the immune cell, such as the targeting of the immune cell (eg increasing its specificity) to the cell(s) involved with the proliferative disorder (eg a tumour cell). For example, a T cell that is used in such embodiments may be modified to alter the specificity of the T cell receptor (TCR) or to introduce antibody-like recognition in chimeric antigen receptors (CARs). CAR immune cells, in particular, are envisioned for use in such embodiments. CAR immune cells are immune cells displaying engineered receptors, which graft an arbitrary specificity (eg to a tumour cell) onto an immune effector cell (eg a T cell). Typically, these receptors are used to graft the specificity of a monoclonal antibody onto a T cell; with transfer of their coding sequence facilitated by retroviral vectors. CAR T cells are a promising therapy for cancer (Song et al 2015, Oncotarget. 6:21533): using ACT, T cells are removed from an individual (typically the subject) and modified so that they express receptors specific to the patient's particular cancer. These T cells, which can then recognise the subject's cancer cells, are (re)introduced into the subject, leading to exposure of TNF (eg produced by the CAR T cells) to the tumour cells and hence killing of such cells, in particular such cells that are sensitised to such TNF-mediate cytotoxicity by exposure to (eg following administration to the subject of) a compound (or pharmaceutical composition) of the invention. Accordingly, in particular of such embodiments, the immune cells can be a CAR T cell, such as one engineered to have increased specificity to the subject's cells that are involved with the proliferative disorder (such as tumour cells).

In alternative embodiments, the exposure of the cells involved with the proliferative disorder to TNF (eg endogenous TNF) may be induced by other means or procedures. Accordingly, in such embodiments, the exposure of the cells involved with the proliferative disorder to (eg an effective amount of) TNF can be induced by (and/or the increase in TNFR1-signalling (and/or TNFR2-signalling) in/of the cells involved with the proliferative disorder is induced by) a pharmaceutical, therapeutic or other procedure that increases the amount of TNF in the plasma of the subject and/or in the environment of such cells.

In certain embodiments, such induced exposure to TNF may be brought about by the administration of a cancer immunotherapy.

In one example, such induced exposure to TNF is brought about by an anti-tumour vaccine (eg, a cancer vaccine). Such cancer vaccines include those whereby antigens (eg, those specific to or preferentially expressed by cancer cells) are directly or indirectly introduced into the subject so as to raise or increase an immune response (typically, an adaptive immune response) in the subject that is envisioned to be (more) specific to the cancer cell. Cancer vaccine may comprise, for example, attenuated viruses, in particular for use against cancers such as cervical or liver cancers that are caused by such virus (eg HPV or HBV). Cancer vaccines can alternatively represent individual (or combinations) of particular tumour antigens (eg, those specific to or preferentially expressed by cancer cells), such as tumour-associated antigens (TAAs) that are used to immunise the subject so as to also raise or increase the immune response in the subject. The cancer vaccine may comprise recombinant protein representing (eg a peptide from) the TAA(s), or may be a tumour specific carbohydrate antigen, and hence are directly introduced into the subject upon administration. The cancer vaccine may, alternatively, comprise a nucleic acid (such as DNA or mRNA) than encodes the protein (or peptide) TAA, and upon administration of the nucleic acid vaccine into the subject, the encoded TAA is expressed by cellular targets in the subject, and hence are indirectly introduced into the subject. TAAs may be divided into two categories: shared tumour antigens; and unique tumour antigens. Shared antigens are expressed by many tumours. Unique tumour antigens result from mutations induced through physical or chemical carcinogens (also known as neoantigens); they are therefore expressed only by individual tumours. The person of ordinary skill will be aware of examples of cancer vaccines in clinical trials, or approved for use, and include PROSTVAC (Bavarian Nordic), PROVENGE (Dendreon) and CV9104 (CureVac), as well as being aware of various TAAs (including neoantigens) and approaches by such tumour antigens may be utilised in cancer vaccines. As further examples: (1) immunisation with recipient-derived clonal myeloma immunoglobulin, idiotype (Id), as a tumour antigen, conjugated with keyhole limpet hemocyanin (KLH) has been shown to produce substantial amount or pro-inflammatory cytokines including TNF (Foglietta et al 2013, Bone Marrow Transplant 48: 269); and (2) a synthetic micro-consensus SynCon DNA vaccine of WT1 antigens induced new, neo-antigen-like responses that were superior to those induced by native WT1 DNA immunogens, such as strong CD4 and CD8 T cell responses (including IFN-gamma, CD107a, and TNF responses).

In another example, such induced exposure to TNF may be brought about by the administration of a ligand (such as an antibody, eg, a monoclonal antibody), for example one that binds to the surface of the cell(s) involved with the proliferative disorder (such as a tumour cell), for example by binding to a TAA or a receptor on the surface of such cell. Cell surface receptors are common targets for such ligand (antibody) therapies and include CD52 and CD20. Once bound to such a cancer antigen, the eg antibodies can induce antibody-dependent cell-mediated cytotoxicity, activate the complement system, or prevent a receptor from interacting with its ligand, all of which can lead to cell death. Approved such ligands that are antibodies include alemtuzumab, ofatumumab and rituximab. In certain embodiments, such ligands used in combination with a compound (or pharmaceutical composition) of the invention can include those that activate T cells or other cell-mediated immune response. For example: (1) anti-CD137 monoclonal antibodies can dramatically promote proliferation of cytokine-induced killer (CIK) cells and expression of TNF (Zhu et al 2009, Biomed Pharmacother 63:509); (2) an agonist anti-OX40 monoclonal antibody can enhance antitumour immune response by augmenting T-cell differentiation (Redmond et al 2014, Cancer Immunol Res. 2014, 2:142); and (3) an anti-ICOS antibody that activates T cells (eg Deng et al 2004, Hybrid Hybridomics 23:176).

In yet another example, the ligand that is administered to the subject is one that binds to an immune (inhibitory) checkpoint molecule. For example, such checkpoint molecule may be one selected from the group consisting of: A2AR, B7-H3, B7-H4, CTLA-4, IDO, KIR, LAG3, PD-1 (or one of its ligands PD-L1 and PD-L2), TIM-3 (or its ligand galectin-9), TIGIT and VISTA. In particular of such embodiments, the ligand binds to a checkpoint molecule selected from: CTLA-4, PD-1 and PD-L1. In other more particular embodiments, the ligand is an antibody selected from the group consisting of: ipilimumab, nivolumab, pembrolizumab, BGB-A317, atezolizumab, avelumab and durvaluma; in particular an antibody selected from the group consisting of: ipilimumab (YERVOY), nivolumab (OPDIVO), pembrolizumab (KEYTRUDA) and atezolizumab (TECENTRIQ). In other embodiments, the ligand that binds to a immune (inhibitory) checkpoint molecule may be a non-antibody peptide, such as a high-affinity PD-1 variant (eg, Maute et al, 2015; PNAS 112:E6506), a peptide targeting the immune checkpoint molecule (such as AUNP-12 of Aurigene Discovery Technologies, US 2011/0318373) or a D peptide blocking an interaction between immune checkpoint molecule (such as the PDL1-PD1 interaction and (D) PPA-1, Chang et al, 2015; Anyeg Chem Int 54:11760). In yet other embodiments, the ligand that binds to an immune (inhibitory) checkpoint molecule may be a small molecule, such as the PDL1-targeting BMS-202 or BMS-8 (Zak et al 2016; Oncotarget 7:30323), the inhibitors of PDL1/D1 known as BMS-1001 or BMS-1166 (Skalniak et al, 2017; Oncotarget 8:72167), the PDL1 and VISTA antagonist CA-170 of Curis/Aurigen undergoing phase 1 trials (Powderly et al, Ann Onc 28: Issue suppl 5, mdx376.007) or CA-327 of Curis/Aurigen which targets PDL1 and TIM3.

In yet another particular embodiment, such induced exposure to TNF may be brought about by radiotherapy.

Radiotherapy is a method of locoregional treatment of cancers or tumours, using radiation to destroy the cancer cells by blocking their ability to multiply and/or to stimulate an immune reaction against them (such one raised as a response to the presence of dead or dying cancer cells). Radiotherapy, in the context of the present invention, consists—in particular—of the therapeutic use of ionising radiation. Said radiotherapy and the associated ionising radiation are those commonly used and known to those skilled in the art. Radiotherapy includes in particular the use of ionizing radiation, for example gamma-rays, X-rays and/or radiation emanating from radioisotopes. In the context of the present invention, it is more particularly X-ray radiation. The radiotherapy may be administered in fractionated form during one or more cycles, such as a cycle that can range from 1 to 4 weeks, more particularly 3 weeks. The cycle defines the interval between the beginning and the end of an administration scheme. When the cycle takes three weeks, radiotherapy can be administered over three weeks, with one week between. The radiotherapy may in particular be administered at a rate of one daily irradiation, 5 days out of 7, for the desired number of weeks. The amount of radiation used in (photon) radiation therapy is measured in gray (Gy), and varies depending on the type and stage of cancer being treated. For curative cases, the typical dose for a solid epithelial tumour ranges from 60 to 80 Gy, while lymphomas are treated with 20 to 40 Gy.

When a compound (or pharmaceutical composition) of the invention (or a compound used in the fifth aspect of the invention) is used in combinations treatments together with any of such other procedures (eg, the other agent, the cancer immunotherapy, the cancer vaccine, the antibody or the radiotherapy, in each case as described herein), then such combination treatment regimen may comprise embodiments where such exposures/administrations are concomitant. In alternative embodiments such administrations may be sequential; in particular those embodiments where the compound (or pharmaceutical composition) is administered before such other procedure. For example a compound (or pharmaceutical composition) of the invention (or a compound used in the invention) may be sequentially administered within about 14 days of (eg before) the other procedure.

Without being bound to theory, administration of a compound (or pharmaceutical composition) of the invention (and hence inhibition of the expression, amount, function, activity or stability of a kinase/key-kinase such as SIK3, eg in a tumour cell) prior to administration of the TNF, TNF variant or TNFR1 or TNFR2 agonist, or prior to administration of such other procedures (eg, the other agent, the cancer immunotherapy, the cancer vaccine, the antibody or the radiotherapy, is foreseen to be particularly effective in sensitising the cells involved with the proliferative disorder to the cytotoxic effects of the cell-mediated immune response.

As described above, existing therapies (or those under clinical trials) involving administration of TNF and/or use of anti-TNF molecules suffer certain known disadvantages; and particular side effects. The present invention provides methods that may be used to mitigate (or reduce) such disadvantages and/or particular side effects.

In a sixth aspect, and as may be further described, defined, claimed or otherwise disclosed herein, the invention relates to a method for the increase of the therapeutic index of treatment with TNF in a subject being treated therewith for a proliferative disorder (eg a cancer disease or a tumour), the method comprising administering a compound (or pharmaceutical composition) of the invention to the subject.

In a related aspect, the invention relates to a method for supporting TNF therapy in a subject suffering from a proliferative disorder (eg a cancer disease or a tumour), the method comprising administering a compound (or pharmaceutical composition) of the invention to the subject.

In a seventh aspect, and as may be further described, defined, claimed or otherwise disclosed herein, the invention relates to a method for the sensitisation of a subject suffering from a proliferative disorder (eg a cancer disease or tumour) to a therapy involving the administration of TNF to the subject, the method comprising administering a compound (or pharmaceutical composition) of the invention to the subject.

The term “sensitisation” (and the like), as used herein in the context of a subject being sensitised to a therapy (eg one involving the administration of TNF), will be understood by the person of ordinary skill, and includes the meaning that the subject increases susceptibility to one or more (treatment) effect—in particular an efficacy effect —that such therapy may have on the subject. In particular, a subject that is so sensitised may, when undergoing such therapy, show an increased response (such as more rapidly, a greater degree of response and/or upon a lower amount or exposure of such therapy) than an analogous subject that have not been so “sensitised”.

In an eighth aspect, and as may be further described, defined, claimed or otherwise disclosed herein, the invention relates to a method for the reduction in risk of (developing) a haematological proliferative disorder (eg, as a secondary disorder) in a subject being treated with an anti-TNF agent, the method comprising administering a compound (or pharmaceutical composition) of the invention to the subject. For example, such aspect may alternatively, be considered as a method for the prevention of a haematological proliferative disorder (as a secondary disorder) in a subject being treated with an anti-TNF agent, the method comprising administering a compound (or pharmaceutical composition) of the invention to the subject.

This aspect of the invention is based on the observation as described above, that there are reports of patients receiving anti-TNF biologics developing lymphomas and other haematological malignancies. Indeed, such disorders are typically described in package leaflets/prescribing information as possible (but rare) side-effects of treatment with anti-TNF agents. As a direct consequence of the perceived increase in haematological malignancy and widespread use of these and other immunosuppressive agents, the WHO classification of tumours now includes the category “iatrogenic immunodeficiency-associated lymphoproliferative disease”.

Therefore, typically in such aspects, the subject is being treated with the anti-TNF agent for an indication other than a proliferative disorder, and in particular of such embodiments the subject does not—upon commencement of the anti-TNF treatment—suffer from a haematological proliferative disorder. Indeed, typically the subject would suffer from, and/or is being treated with the anti-TNF agent for an autoimmune disorder; preferably an autoimmune disorder selected from the group consisting of: rheumatoid arthritis, psoriatic arthritis, ankylosing spondylitis, plaque psoriasis, inflammatory bowel disease, ulcerative colitis, Crohn's Disease, psoriasis, hidradenitis suppurativa and refractory asthma; such as one selected from the group consisting of: rheumatoid arthritis, psoriatic arthritis, ankylosing spondylitis, plaque psoriasis and Crohn's Disease; and in particular rheumatoid arthritis.

In certain embodiments, the anti-TNF agent is one selected from a list consisting of: infliximab, adalimumab, golimumab, humicade, etanercept, onercept and certolizumab pegol, in particular infliximab or humicade.

In certain embodiments, the haematological malignancies proliferative disorder may be a lymphoproliferative disease, in particular an iatrogenic immunodeficiency-associated lymphoproliferative disease.

In certain embodiments of such sixth to eighth aspects, the (treatment) effect (eg the increase in therapeutic index, sensitisation of a subject or reduction in risk) is mediated by (eg, the treatment comprises, is by, is mediated by or involves): (i) inhibiting a kinase (eg a key-kinase such as SIK3) (such as by the inhibition of the function and/or activity of phosphorylated SIK3), in particular by inhibiting such a kinase (eg a key-kinase) in cells involved with the proliferative disorder; and/or (ii) sensitising such cells to the killing (apoptotic/cytotoxic) effects of TNF. In further embodiments, the (treatment) effect may not be mediated by (eg, the treatment may not comprise or involve) inhibiting one or more other key-kinases (eg, ABL1 and/or SRC, or SIK2 and/or SIK1), in particular not mediated by (eg, the treatment does not comprise or involve) inhibiting or more other key-kinases (eg SIK2 and/or SIK1 (and/or SIK3) in immune cells.

### Pre-Clinical and Clinical Testing

In certain embodiments, the subject is a human volunteer; for example, one that has chosen (eg consented) to be administered a compound (or pharmaceutical composition) of the invention for a clinical trial or other experimental use of the compound. Such a human volunteer may be a healthy human (eg, a healthy volunteer) or may be suffering from a disorder such as a proliferative disorder (eg, a cancer patient). In another embodiment, the subject is a laboratory animal, in particular an animal selected from the group consisting of: mouse, rat, rabbit, pig and monkey.

In such (eg experimental treatment) embodiments, a plurality of such subjects can be treated; in particular 5 or more subjects, such as between about 5 and 20, 10 and 50, 25 and 200, or 75 and 250 subjects, or more than about 250 subjects.

Such experimental (or clinical trial) treatments may comprise: (i) the administration to at least one of such subjects of one dosage of a compound of the invention and/or one formulation of a pharmaceutical composition of the invention; and (ii) the administration to at least one other of such subjects of a different dosage of the compound and/or a different formulation of the pharmaceutical composition.

In further of such embodiments, such experimental (or clinical trial) treatments may comprise: (i) the administration to at least one of such subjects of one dosage of a compound of the invention and/or one formulation of the pharmaceutical composition of the invention; and (ii) the administration to at least one other of such subjects of either: (a) a placebo; or (b) the dosage of the compound and/or the formulation of the pharmaceutical composition of the subject(s) of (i) as well as an additional pharmaceutical, therapeutic or other procedure.

The term “placebo” will be art recognised, and includes a substance or treatment of no intended therapeutic value. In such embodiments, the placebo can be made to resemble the other administration so that it functions as a control, such as in a blinded trial.

In certain of such embodiments, such experimental (or clinical trial) treatment is specifically designed for the investigation and/or determination of a therapeutically effective dosage of a compound of the invention and/or the identification of a therapeutically effective formulation of a pharmaceutical composition of the invention.

### Diagnosis

In a ninth aspect, the invention relates to a method of diagnosing and treating a disease, disorder or condition characterised by the presence of or an amount of, and/or characterised by (eg aberrant) expression or activity of, one or more applicable biomarkers (such as a kinase) in a subject, such as a human patient, comprising:
detecting one or more such applicable biomarkers in a biological sample from said subject, thereby diagnosing if the subject is suffering (or is likely to suffer) from such a disease, disorder or condition; and
administering an effective amount of a compound of the invention (and/or a pharmaceutical composition comprising such compound) to the so diagnosed subject, in particular practicing a treatment method of the invention on the subject.

In one of such embodiments, the disease, disorder or condition is a proliferative disorder, such as one disclosed elsewhere herein (eg a tumour or cancer).

The term “applicable biomarker” means any one (or more) of the genes expressed by the cell involved with the proliferative disorder that are involved in the (eg kinase/key-kinase mediated) cellular resistance against an immune response (eg a cell-mediated immune response such as TNF). Such genes include: (X) one or more kinase, in particular one or more key-kinase as described herein, such as one selected from the group consisting of: SIK3, SIK2, SIK1, ABL/BCR-ABL, KIT, NEK2, BRAF. CSF1R, HCK, TEC-family kinases (eg BTK, TXK or ITK), AXL, BLK, TYRO3, MERTK, ZAP70, SYK, EGFR and BRK; and/or selected from the group consisting of: FLT3, LCK, PHA2, EPHA4, ACK1, NEK11, WEE1, WNK2, Aurora-A, Aurora-B and TBK1, 3 or in particular, SIK3 or phosphorylated SIK3; (Y) a mutant of a kinase, such as a mutant ABL1 kinase (eg BCR-ABL) or a mutant of KIT kinase; and/or (Z) one or more of (a) to (f) below):
(a) TNFR1 (or TNFR2), such as the presence of (or an amount of) or expression and/or activity of TNFR1 (or TNFR2), in particular TNFR1;
(b) LKB1, such as the presence of (or an amount of) or expression and/or activity of LKB1, in particular increased amount or activity of LKB1 or pLKB1;
(c) one or more class II (eg IIa) HDACs, eg HDAC4, such as the presence of (or an amount of) or expression and/or activity of such HDAC, in particular increased amount or activity of such HDAC or pHDAC, especially in the cytoplasm of cells of the tumour;
(d) Expression of NF-kappa-B, in particular, constitutive expression of NF-kappa-B;
(e) NF-kappa-B, such as the presence of (or an amount of) or expression and/or activity of NF-kappa-B, in particular increased amount or activity of NF-kappa-B or acetylated NF-kappa-B, especially in the nucleus of cells of the tumour; and/or
(f) one or more anti-apoptotic genes, such as the presence of (or an amount of) or expression and/or activity of one or more anti-apoptotic genes, in particular one or more of such genes under transcriptional control by NF-kappa-B.

In certain embodiments, the applicable biomarker is one or more key-kinases selected from the list consisting of: BRAF, NEK2, PRK2, PKC, and in particular KIT, RIPK2, ABL2 and PDGF-alpha: and/or selected from the list consisting of: TAOK2, SYK, TYRO3, ACVR2B, MEKK2, AXL, ITK, MAP3K11, TRKA, MERTK, ZAP70, and MEKK2, and in particular CSF1R, HCK, TXK, YES, LCK, SRC, EPHA1 and FGR.

In a certain of such embodiments, the applicable biomarker is CSF1R or HCK, BTK, TXK, ITK or BRK.

In another embodiment of the above method of diagnosing and treating, the “applicable biomarker” is, alternatively, TGFbeta. In particular, those subjects having tumour types with high TGFbeta content (Hsing et al 1996, Cancer Res 56:5146) such as breast, lung, prostate, liver cancer, or a lymphoma, may be suitable for treatment with a compound disclosed herein (eg a compound of any of Formulae (I), (Ia), (II), (III), (IV), (V), (Va), (VI), (VII), (VIIa), ((VIII), (IX), (X), (XI) or (XII), for example by the compound inhibiting SIK3 and rendering sensitive to apoptosis the tumour cells. Recently, SIKs (particularly SIK3) have been demonstrated to also regulate TGFbeta-mediated transcriptional activity and apoptosis, with Hutchinson et al (2010, Cell Death and Disease 11:49) showing that SIK3 expression or activity results in resistance to TGFbeta-mediated apoptosis. TGFbeta is a member of the cytokine family that binds to its cognate receptors on cells and mediates multiple cellular processes ranging from proliferation, differentiation, migration, apoptosis to epithelial-mesenchymal transition (Massagud et al 2012, Nat Rev Mol Cell Biol 13:616). In a tumour setting, the TGFbeta axis can be mis-regulated; leading to oncogenic processes (Drabsch & ten Dijke 2012, Cancer Metastasis Rev 31:553). The TGFbeta axis can serve to limit immune responses by inhibiting the expression of pro-apoptotic and cytolytic factors such as granzymes, perforins, IFNgamma in T cells or NK cells. Therefore, targeting TGFbeta in oncology has recently gained prominence. However, TGFbeta can also induce apoptosis in tumour cells via the caspase pathway. Indeed, tumour cell lines from lymphoma (Inman & Allday 2000, J Immunol 165:2500), liver cancer (Kim et al 2002, Mol Cell Biol 22:1369) and prostate cancer (http Hsing et al 1996, Cancer Res 56:5146) have been shown to be sensitive to TGFbeta-mediated apoptosis.

In one aspect, the invention relates to a method for determining that a subject suffering from a proliferative disorder is suitable for treatment with a compound or pharmaceutical composition as defined elsewhere herein, in particular wherein the compound is selected from the following compounds (a) or (b), or the pharmaceutical composition comprises such a compound and, optionally, a pharmaceutically acceptable excipient: (a) a compound of formula (I), (Ia), (II), (III), (IV), (V), (Va), (VI), (VII), (VIIa), ((VIII), (IX), (X), (XI) or (XII), such as any embodiment thereof as described above; or (b) ARN-3261 (Vankayalapati et al 2017, AACR Cancer Res 77(13 Suppl):Abstract nr LB-296; U.S. Pat. Nos. 9,260,426, 9,890,153, 9,951,062).

Such a determining method can comprise, determining in a biological sample that has been (previously) obtained from said subject, (X) the presence of (and/or an amount of) MEF2C protein, such as of phosphorylated MEF2C protein and/or of MEF2C protein as an active transcription factor; preferably wherein the proliferative disorder is further characterised by the presence of (and/or an amount of) phosphorylated HDAC4 protein, such as of HDAC4 protein phosphorylated by SIK3; and/or (Y) (i) the presence of a human chromosomal translocation at 11q23; (ii) the presence of a rearrangement of the KMT2A gene; (iii) the presence of (and/or an amount of) a KMT2A fusion oncoprotein; and/or (iv) the presence of a mutation in the KRAS gene and/or in the RUNX1 gene. For example, the protein or oncoprotein may be present at an amount (eg a quantitative amount), such as an amount that is in excess of physiological amount (eg, for that cell type and/or that time/stage), including from expression or over-expression of the protein. In another embodiment, such protein may be present at an amount (eg a quantitative amount) that is in excess of a threshold amount or is an outlier from a reference distribution of amounts of such protein/oncoprotein.

In one embodiment of such method, the biological sample that had (previously) been obtained from the subject comprises cells that are involved with the proliferative disorder (eg, cancer or tumour cells).

Such a method is, for example, conducted as an in-vitro method; such as a method that is not practiced on the human or animal body (eg, is not practiced on the body of such subject).

The presence of (and/or the amount of) said protein, translocation, rearrangement, oncoprotein and or mutation (as applicable) in the biological sample can indicate that the subject is suitable for treatment with the compound or pharmaceutical composition.

Certain embodiments of such determining methods comprise those embodiments of said protein, translocation, rearrangement, oncoprotein and or mutation as are described elsewhere herein.

In other embodiments (and as may be further specified elsewhere herein), the proliferative disorder may be a cancer or a tumour, for example a haematopoietic malignancy and/or a lymphoid malignancy. In particular, the proliferative disorder may be: (i) a myeloma, preferably multiple myeloma; or (ii) a leukaemia, preferably an acute myeloid leukaemia (AML) or an acute lymphoblastic leukaemia (ALL), more preferably T cell acute lymphoblastic leukaemia (T-ALL), an MLL-AML or an MLL-ALL. The proliferative disorder may be one set forth in FIG. 13.

The subject in such determining methods may be, for example, a human paediatric patient and/or may be a subject carrying a KMT2A rearrangement (KMT2A-r); In particular embodiments, the subject may be a patient suffering from a KMT2A-r leukaemia, especially a (eg, paediatric) human patient as described elsewhere herein.

In certain embodiments, such determining method can further comprise administering the compound (eg, a compound selected from the compounds (a) or (b)) to the subject, in particular when the presence of (and/or the amount of) said protein, translocation, oncoprotein and or mutation is determined in a biological sample that had been obtained from said subject. Such embodiment may, alternatively, be described as an additional aspect of the invention that provides a method of determining (the suitability for) and treating the subject, such method comprising the steps of such embodiment.

Further embodiments of the administering (or treatment) step of this method of diagnosis and treatment are described in more details elsewhere; as are particular embodiments of the methods of the detection, determination or diagnostic method step of this method. Particular of such embodiments include those where the amount of a compound (and/or pharmaceutical composition) of the invention administered to the subject is correlated to the plasma or intratumoural concentration of TNF (in the subject), wherein a greater amount (or dose) of the compound (and/or pharmaceutical composition) administered to such subject in those cases of a greater plasma or intratumoural concentration of TNF.

In certain embodiments, a biological sample will (preferably) comprise cells or tissue of the subject, or an extract of such cells or tissue, in particular where such cells are those (usually, typically; or in the case or a specific subject as suspected to be) involved with the proliferative disorder (eg tumour cells such as cells of a solid tumour). The tumour or cell thereof, may be one of, or derived from, one of the tumours described elsewhere herein.

In particular embodiments of such aspect, the method will also comprise a step of:
providing (such as by obtaining) the biological sample from the subject, in particular where such step is conducted prior to the detection step.

In particular embodiments, such detection and/or determination methods can be practiced as a method of diagnosis, such as a method of diagnosis whether a mammalian subject (such as a human subject or patient) has a disease, disorder or condition, in particular (the presence of) a proliferative disorder such as a cancer or tumour (or has a risk of developing such a disease, disorder or condition) that is associated with cellular resistance against a cell-mediated immune response and/or that is associated with (eg aberrant) expression or activity of the applicable biomarker (eg SIK3); in particular a (solid) tumour, such as one having cellular resistance against a cell-mediated immune response.

In certain embodiments of these detection, determination and/or diagnostic methods, the cellular resistance against a cell-mediated immune response is cellular resistance against a Tcell-mediated immune response, in particular cellular resistance to the killing (apoptotic/cytotoxic) effect of TNF and/or of TNFR1- or TNFR2-signalling.

Accordingly, particular embodiments of these detection and/or diagnostic methods may also comprise a step of determining the presence or amount of TNF in the sample, wherein the presence of (or an amount of) TNF in the sample indicates a/the proliferative disorder (or a/the risk of developing a proliferative disorder) that is associated with cellular resistance against the cell-mediated immune response, and/or associated with (aberrant) expression or activity of the kinase (eg SIK3), in the subject. In particular of such embodiments, amount of TNF in the sample is determined qualitatively. Preferably, the subject is distinguished as having: (i) a plasma concentration of TNF greater than about 2 pg/mL or 5 pg/mL in a plasma sample from the subject; and/or (ii) an intratumoural concentration of TNF greater than about 0.5 pg/mL or 1 pg/mL from a tissue sample from the subject, indicates the (presence of the proliferative disorder or a risk of developing the proliferative disorder that is associated with cellular resistance against the cell-mediated immune response, and/or associated with expression or activity of the kinase (eg SIK3), in the subject.

Methodologies to determine the presence or amount of TNF in a sample are described elsewhere herein (in particular, quantitative detection of TNF using ELISA assays such as a Quantitkine TNF-alpha Immunoassay; as are amounts of TNF that, if are exceeded by the TNF present in the sample, indicate that a proliferative disorder associated with cellular resistance against the cell-mediated immune response, and/or associated with (aberrant) expression or activity of the kinase (eg SIK3), in the subject.

In certain embodiments, the biological sample is one obtained from a mammalian subject like a human patient. The term “biological sample” is used in its broadest sense and can refer to a bodily sample obtained from the subject (eg, a human patient). For example, the biological sample can include a clinical sample, ie, a sample derived from a subject. Such samples can include, but are not limited to: peripheral bodily fluids, which may or may not contain cells, eg, blood, urine, plasma, mucous, bile pancreatic juice, supernatant fluid, and serum; tissue or fine needle biopsy samples; tumour biopsy samples or sections (or cells thereof), and archival samples with known diagnosis, treatment and/or outcome history. Biological samples may also include sections of tissues, such as frozen sections taken for histological purposes. The term “biological sample” can also encompass any material derived by processing the sample. Derived materials can include, but are not limited to, cells (or their progeny) isolated from the biological sample, nucleic acids and/or proteins extracted from the sample. Processing of the biological sample may involve one or more of: filtration, distillation, extraction, amplification, concentration, fixation, inactivation of interfering components, addition of reagents, and the like. In certain embodiments, the biological sample may comprise cells that are involved with a disorder (eg, a proliferative disorder) such as cancer or tumour cells).

In some embodiments, these detection, determination and/or diagnostic methods may be a computer-implemented method, or one that is assisted or supported by a computer. In some embodiments, information reflecting the presence or an amount of the applicable biomarker (eg, a key-kinase such as ABL1/BCR-ABL, SRC and/or SIK3) to be determined (or activity thereof) in a sample is obtained by at least one processor, and/or information reflecting the presence or an amount of such marker (or activity thereof) in a sample is provided in user readable format by another processor. The one or more processors may be coupled to random access memory operating under control of or in conjunction with a computer operating system. The processors may be included in one or more servers, clusters, or other computers or hardware resources, or may be implemented using cloud-based resources. The operating system may be, for example, a distribution of the Linux™ operating system, the Unix™ operating system, or other open-source or proprietary operating system or platform. Processors may communicate with data storage devices, such as a database stored on a hard drive or drive array, to access or store program instructions other data. Processors may further communicate via a network interface, which in turn may communicate via the one or more networks, such as the Internet or other public or private networks, such that a query or other request may be received from a client, or other device or service. In some embodiments, the computer-implemented method of detecting the presence or an amount of the applicable biomarker (or activity thereof) in a sample is provided as a kit.

Such detection, determination and/or diagnosis methods can be conducted as an in-vitro (eg ex-vivo) method, and can be, for example, practiced using the kit of the present invention (or components thereof). An in-vitro method may use, involve or be practised on immortalised cell lines (such as those replicated, cultured or indefinitely maintained outside of the body of an animal or human), or it may be use, involve or be practised in-vitro using cells (such as primary cells) directly or freshly obtained from the body of an animal of human (eg, practised as a so-called “ex-vivo” method).

In some embodiments of these detection, determination and/or diagnosis methods, the biological sample is a tissue sample from the subject, such as a sample of a tumour or a cancer from the subject. As described above, such tissue sample may be a biopsy sample of the tumour or a cancer such as a needle biopsy sample, or a tumour biopsy section or an archival sample thereof. Such a tissue sample may comprise living, dead or fixed cells, such as from the tumour or a cancer, and such cells may be suspected of expressing (e.g. aberrantly or localised) the applicable biomarker to be determined.

In some embodiments, determination and/or diagnosis method of the invention can comprise, such as in a further step, comparing the detected amount (or activity of) of (eg protein or mRNA of) the applicable biomarker (eg the kinase/key-kinase such as SIK3, and in particular phosphorylated SIK3) with a standard or cut-off value; wherein a detected amount greater than the standard or cut-off value indicates a phenotype (or a risk of developing a phenotype) that is associated with cellular resistance against the cell-mediated immune response in the subject and/or is associated with is associated with (aberrant) expression or activity of the kinase/key-kinase (eg SIK3) in the subject. Such a standard or cut-off value may be determined from the use of a control assay, or may be pre-determined from one or more values obtained from a study or a plurality of samples having known phenotypes. For example, a cut-off value for a diagnostic test may be determined by the analysis of samples taken from patients in the context of a controlled clinical study, and determination of a cut-off depending on the desired (or obtained) sensitivity and/or specificity of the test.

The applicable biomarker can, in certain embodiments, be detected by detecting protein of the applicable biomarker, or by detecting mRNA that encodes protein of the applicable biomarker. Methods to detect proteins (eg antibody detection, in particular by immunohistochemistry) and mRNA (eg by hybridisation, qPCR or sequencing) are well known.

Examples of methods useful in the detection of (such as the presence or absence of, or an amount of) the applicable biomarker (such as the kinase/key-kinase eg SIK3, and in particular phosphorylated SIK3) include immunoassays, such as the enzyme linked immunosorbent assay (ELISA) and the radioimmunoassay (RIA), which employ an antigen binding protein (“ABP”) such as an antibody or an antigen-binding fragment thereof, that specifically binds to such applicable biomarker.

### Skin Pigmentation and Related Aspects

The role of salt inducible kinase 2 (SIK2) as an inhibitor of melanogenesis via the suppression of the cAMP-response element binding protein (CREB)-specific coactivator 1 (TORC1) is described by Kumagai et al (2011), and that a potent inhibitor of SIK2 was able to promote melanogenesis in B16F10 Melanoma Cells. Mujahid et al (2017) was able to confirm that topical treatment of human skin explants with the SIK2 inhibitor HG-9-91-01 (and other SIK2 inhibitors structurally related to those of the present invention) was able to induce eumelanisation in such human skin. Example 12 herein describes analogous investigations into the promotion of melanogenesis in (human) skin cells by compounds of formulae (I), (Ia), (II), (III), (IV), (V), (Va), (VI), (VII), (VIIa), ((VIII), (IX), (X), (XI) or (XII).

Accordingly, in another aspect, the invention relates to a method of increasing skin pigmentation (or of increasing the appearance of skin pigmentation) in a subject, the method comprising administering to the subject an (eg effective) amount of a kinase inhibitor as specified under the heading “Compounds”, or a pharmaceutical composition thereof. An amount (such as an effective amount) of the compound (or pharmaceutical composition), in this context, is one that refers to an amount sufficient to elicit the desired biological response (eg, the degree of skin pigmentation, or the appearance of skin pigmentation).

In one embodiment of such aspect, this method is not practiced as a method for treatment of the human or animal body by surgery or therapy (or a diagnostic method) practised on the human or animal body. For example, the method is practised for cosmetic purposes, such as to increase the production of melanin in the skin (and hence the skin colour) of the subject for aesthetic reasons.

In another embodiment of such aspect, it is practiced as a method of increasing skin pigmentation (or of increasing the appearance of skin pigmentation) in a subject for medical purposes (eg for treatment, including to prevent). For example, the method may be practiced to increase skin pigmentation (eg, without a requirement for UV irradiation), to improve UV protection and for example to reduce the risk of skin cancer. For example, in such contexts term “prevent” can refer to a prophylactic treatment of a subject who is not and did not have a disease but is at risk of developing the disease or who had a disease, does not have the disease, but is at risk of regression of the disease. In certain of such embodiments, the administered amount of the compound or pharmaceutical composition may be one that is “prophylactically effective”. A “prophylactically effective amount” of a compound (or pharmaceutical composition) described herein can be an amount sufficient to prevent a condition, or one or more symptoms associated with the condition or prevent its recurrence. For example, a prophylactically effective amount in this context can mean an amount of a therapeutic agent, alone or in combination with other agents, (or those present in one or more pharmaceutical compositions) which provides a prophylactic benefit in the prevention of skin cancer. The term “prophylactically effective amount” can encompass an amount that improves overall prophylaxis or enhances the prophylactic efficacy of another prophylactic agent.

Accordingly, in a related aspect the invention relates to a compound for use, or a pharmaceutical composition for use, in a treatment of increasing skin pigmentation (or of increasing the appearance of skin pigmentation) in a subject, the treatment comprising administering the compound or the pharmaceutical composition to the subject, wherein the compound is selected from a a kinase inhibitor as specified under the heading “Compounds”.

In certain embodiments of such aspects, the compound is any compound of formula (I), (Ia), (II), (III), (IV), (V), (Va), (VI), (VII), (VIIa), ((VIII), (IX), (X), (XI) or (XII), or ARN-3261 (Vankayalapati et al 2017, AACR Cancer Res 77(13 Suppl):Abstract nr LB-296; U.S. Pat. Nos. 9,260,426, 9,890,153, 9,951,062), and any embodiment thereof as specified under the heading “Compounds”.

In certain embodiments of such another and related aspects, the compound (or the pharmaceutical composition comprising the compound) can be topical (or transdermal) administration to the subject. The term “topical” is art-recognised in this context and its meaning includes the local application of the compounds described herein to a body surface of a human or non-human animal. In certain embodiments, the body surface is skin. In certain embodiments, the skin is on a body part. In certain embodiments, the skin is on the face. In certain embodiments, the skin is on the neck. In certain embodiments, the skin is on the torso. In certain embodiments, the skin is on the chest. In certain embodiments, the skin is on the back. In certain embodiments, the skin is on the arms. In certain embodiments, the skin is on the legs.

### Intermediates, Synthesis, Manufacturing and Other Aspects

The compounds disclosed herein can be prepared as described below or prepared by methods analogous thereto, which are readily known and available to one of ordinary skill in the art of organic synthesis.

In a tenth aspect, and as may be further described, defined, claimed or otherwise disclosed herein, the invention relates to an intermediate selected from a compound having formula (Id):

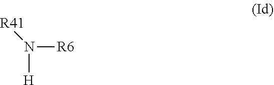

and solvates, salts, complexes, polymorphs, crystalline forms, racemic mixtures, diastereomers, enantiomers, tautomers, conformers, isotopically labeled forms, and combinations thereof, wherein:
R⁶ is any R⁶ described, disclosed or defined elsewhere herein; and
R⁴¹ is selected from the group consisting of H and an amino protecting group.

In one embodiment of the intermediate of the tenth aspect, R⁴¹ is an amino protecting group. For example, the amino protecting group may be selected from the group consisting of tert-butyloxycarbonyl (BOC), 9-fluorenylmethoxycarbonyl (FMOC), benzyloxycarbonyl (Cbz), p-methoxybenzylcarbonyl (MOZ), acetyl (Ac), trifluoroacetyl, benzoyl (Bz), benzyl (Bn), p-methoxybenzyl (PMB), 3,4-dimethoxyphenyl (DMPM), p-methoxyphenyl (PMP), 2,2,2-trichloroethoxycarbonyl (Troc), triphenylmethyl (trityl; Tr), toluenesulfonyl (tosyl; Ts), para-bromophenylsulfonyl (brosyl), 4-nitrobenzenesulfonyl (nosyl), and 2-nitrophenylsulfenyl (Nps).

In an alternative embodiment of the intermediate of the tenth aspect, R⁴1 is H.

In one embodiment of the intermediate of the tenth aspect, the intermediate is selected from (and in an alternative embodiment is not selected from one or more, preferably all of) the group consisting of:

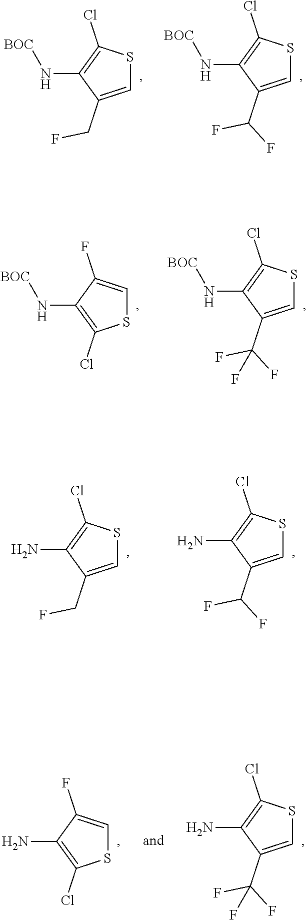

and solvates, salts, complexes, polymorphs, crystalline forms, tautomers, conformers, isotopically labeled forms, and combinations thereof. For example, the intermediate may be selected from (and in an alternative embodiment is not selected from one or more, preferably all of) the group consisting of:

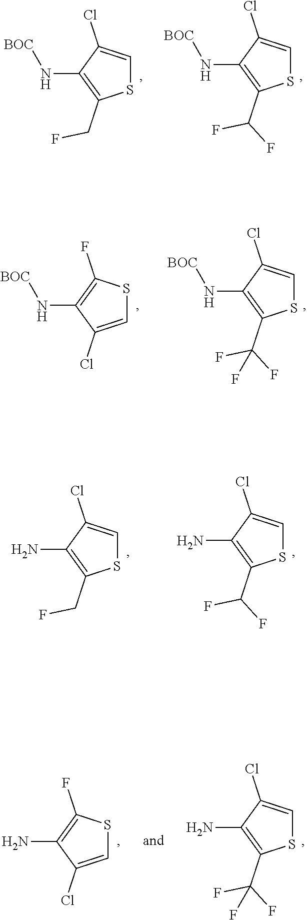

and solvates, salts, complexes, polymorphs, crystalline forms, tautomers, conformers, isotopically labeled forms, and combinations thereof.

In one embodiment, the intermediates of the invention do not encompass compounds of one or more of the following groups (1) to (3) (in the groups (1) to (3) a moiety (such as methyl) is unsubstituted unless it is explicitly specified that said moiety is substituted):
(1) the intermediate is not 2-bromo-4-(trifluoromethyl)thiophen-3-amine; [CAS 1369240-59-4]
(2) when the R⁴⁰ attached to the C ring atom at position 4 of the thienyl ring is -Me, and the other R⁴⁰ is —CHF₂, then R⁴1 is not (1-propylpiperidin-2-yl)carbonyl; and [CAS 2099704-46-6, 2099704-17-1, 2099703-62-3, 2099703-17-8]
(3) when the R⁴⁰ attached to the C ring atom at position 4 of the thienyl ring is -Me, and the other R⁴⁰ is F, then R⁴¹ is not 4,5-dihydro-1H-imidazol-2-yl. [CAS 1369494-03-0]

In an eleventh aspect, and as may be further described, defined, claimed or otherwise disclosed herein, the invention relates to the use of an intermediate of formula (Id) to prepare a compound cyclic urea moiety (in particular a kinase inhibitor, especially an inhibitor of one or more protein kinases selected from the list consisting of: SIK (preferably SIK3), CSFR1, ABL/BCR-ABL, HCK, PDGRF, LCK, SRC, and KrT; preferably one or more protein kinases selected from the list consisting of: SIK3, ABL/BCR-ABL, HCK, and CSF1R kinases, such as a compound of the invention (eg, a compound of formula (I), (Ia), (II), (III), (IV), (V), (Va), (VI), (VII), (VIIa), ((VIII), (IX), (X), (XI) or (XII), or a solvate, salt (in particular a pharmaceutically acceptable salt), N-oxide (in particular, an N-oxide of R^{1a} and/or R⁶), complex, polymorph, crystalline form, racemic mixture, diastereomer, enantiomer, tautomer, conformer, isotopically labeled form, prodrug (in particular a prodrug having at least one derivatized hydroxyl group, as specified above, or a solvate, salt, N-oxide, complex, polymorph, crystalline form, racemic mixture, diastereomer, enantiomer, tautomer, conformer, isotopically labeled form or combination thereof), or combination thereof)), wherein the method comprises reacting the intermediate of formula (Id) with a corresponding reactant (eg, see Example 1.1), and, optionally, removing the amino protecting group.

In one embodiment of the method of the eleventh aspect, the reaction of the intermediate with the corresponding reactant takes place in a solvent, such as an aprotic solvent, e.g., acetonitrile.

In a twelfth aspect, the invention relates to a method for preparing a compound of the invention (eg, a compound of formula (I), (Ia), (II), (III), (IV), (V), (Va), (VI), (VII), (VIIa), ((VIII), (IX), (X), (XI) or (XII)) that is in a (eg substantially) purified form, the method comprising the steps:
providing the compound (eg AA3, AA5, AA6 or AA7) in admixture with one or more impurities; and
removing at least a fraction of the impurities from the admixture.

In certain embodiments of such aspect, suitable methods to remove a fraction of the impurities are well known and include eg column chromatography, selective precipitation, trituration and elution of impurities with a suitable solvent in which the desired compound is not soluble, etc.

The fraction of impurities removed may be such that the compound is prepared in substantially pure form; that is, for example, in a percentage purity as described above.

In other embodiments, the admixture of provided by synthesising an impure form of the compound.

In a thirteenth aspect, the invention also relates to a method for manufacturing a pharmaceutical composition comprising the step of formulating a compound of the invention (eg a compound of formula (I), (Ia), (II), (III), (IV), (V), (Va), (VI), (VII), (VIIa), ((VIII), (IX), (X), (XI) or (XII)) together with a pharmaceutically acceptable excipient (such as one described elsewhere herein, such as a pharmaceutically acceptable stabiliser of carrier).

In a fourteenth aspect, the invention also relates to a method of preparing a pharmaceutical package, comprising the steps:
inserting into packaging a pharmaceutical composition of the invention, thereby forming a package containing the pharmaceutical composition; and optionally,
inserting into the package a leaflet describing prescribing information for the pharmaceutical composition.

In one of such embodiments, the pharmaceutical composition is in finished pharmaceutical form; for example, that is in the form that would be administered (or finally prepared to be administered) to a subject.

The packaging can be primary and/or secondary packaging. For example, the primary packaging may be a glass vial or a blister strip. Typical (but non-limiting) secondary packaging can be a box or carton, which in certain embodiments may be marked with the name, strength and/or brand of the pharmaceutical composition it contains.

The packaging may further comprise a leaflet or other information. In particular, that describing (either to the patient and/or the administering physician) salient information or details on the pharmaceutical composition contained in the package, such as how to administer, recommended dosages, safety and/or side-effect information.

In a fifteenth aspect, the invention also relates to a pharmaceutical package containing a pharmaceutical composition of the invention; preferably, wherein the pharmaceutical composition is in finished pharmaceutical form. In certain embodiments of such aspect, the pharmaceutical package may further containing a leaflet describing prescribing information for the pharmaceutical composition.

In view of the above, it will be appreciated that the present invention also relates to the following itemised embodiments:

ITEM 1. A compound selected from the group consisting of a kinase inhibitor of the formula (I):

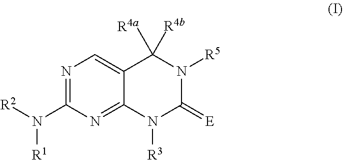

and solvates, salts, N-oxides, complexes, polymorphs, crystalline forms, racemic mixtures, diastereomers, enantiomers, tautomers, conformers, isotopically labeled forms, prodrugs, and combinations thereof; wherein:
R¹ is -Q-R^{1a};
R^{1a} is selected from the group consisting of H, alkyl, alkenyl, alkynyl, cycloalkyl, aryl, heterocyclyl, heteroaryl, halogen, —CN, azido, —NO₂, —OR¹¹, —N(R¹²)(R¹³), —N(R¹¹)(OR¹¹), —S(O)₀₋₂R¹¹, —S(O)₁₋₂OR¹¹, —OS(O)₁₋₂R¹¹, —OS(O)₁₋₂OR¹¹, —S(O)₁₋₂N(R¹²)(R¹³), —OS(O)₁₋₂N(R¹²)(R¹³), —N(R¹¹)S(O)₁₋₂R¹¹, —NR¹¹S(O)₁₋₂OR¹¹, —NR¹¹S(O)₁₋₂N(R¹²)(R¹³), —P(O)(OR¹¹)₂, —OP(O)(OR¹¹)₂, —C(═X)R¹¹, —C(═X)XR¹¹, —XC(═X)R¹¹, and —XC(═X)XR¹¹, wherein each of the alkyl, alkenyl, alkynyl, cycloalkyl, aryl, heterocyclyl, and heteroaryl groups is optionally substituted with one or more independently selected R³⁰;
Q is selected from the group consisting of a bond, alkyl, alkenyl, alkynyl, cycloalkyl, aryl, heterocyclyl, heteroaryl, wherein each of the alkyl, alkenyl, alkynyl, cycloalkyl, aryl, heterocyclyl, and heteroaryl groups is optionally substituted with one or more independently selected R³⁰;

### R² is H;

R³ is selected from the group consisting of a bond, H, alkyl, alkenyl, alkynyl, cycloalkyl, aryl, heterocyclyl, heteroaryl, halogen, —CN, azido, —NO₂, —OR¹¹, —N(R¹²)(R¹³), —N(R¹¹)(OR¹¹), —S(O)₀₋₂R¹¹, —S(O)₁₋₂OR¹¹, —OS(O)₁₋₂R¹¹, —OS(O)_{1- 2}OR¹¹, —S(O)₁₋₂N(R¹²)(R¹³), —OS(O)₁₋₂N(R¹²)(R¹³), —N(R¹¹)S(O)₁₋₂R¹¹, —NR¹¹S(O)₁₋₂OR¹¹, —NR¹¹S(O)₁₋₂N(R¹²)(R¹³), —P(O)(OR¹¹)₂, —OP(O)(OR¹¹)₂, —C(═X)R¹¹, —C(═X)XR¹¹, —XC(═X)R¹¹, and —XC(═X)XR¹¹, wherein each of the alkyl, alkenyl, alkynyl, cycloalkyl, aryl, heterocyclyl, and heteroaryl groups is optionally substituted with one or more independently selected R³⁰;
optionally R¹ and R³ may join together via a group L′ to form a moiety R¹-L′-R³, preferably to form a moiety Q-L′-R³, wherein L′ is selected from the group consisting of a bond, alkylene, alkenylene, alkynylene, —(CH₂)ₚ-[Y—(CH₂)_{q}]ᵣ—, alkenylene —[Y—(CH₂)_{q}]ᵣ—, wherein p is an integer between 1 and 10, q is an integer between 0 and 6, r is an integer between 1 and 3, wherein if q is 0 then r is 1; Y is independently selected from 0, S, and —N(R¹³)—; and each of the alkylene, alkenylene, alkynylene, —(CH₂)ₚ—, and —(CH₂)_{q}— groups is optionally substituted with one or more independently selected R³⁰;
each of R^{4a} and R^{4b} is independently selected from the group consisting of H, C₁₋₈ alkyl, and —(CH₂)ₛ—[O—(CH₂)ₜ]ᵤ—H wherein s is an integer between 0 and 7, t is an integer between 0 and 7, u is an integer between 1 and 3, wherein if t is 0 then u is 1, and the total number of carbon and oxygen atoms of the —(CH₂)ₛ—[O—(CH₂)ₜ]ᵤ—H does not exceed 8; or optionally R^{4a} and R^{4b} may join together to form, together with the carbon to which they are attached, C₃₋₈ cycloalkyl or a 4- to 8-membered heterocyclyl comprising at least one 0 as the only heteroatom element, wherein in case that the 4- to 8-membered heterocyclyl comprises more than one 0, different 0 are not directly bound to each other;
R⁵ is -L-R⁶;
L is selected from the group consisting of a bond, C₁₋₆ alkylene, C₂₋₆ alkenylene, C₂₋₆ alkynylene, and —(CH₂)ₘ—[Y—(CH₂)ₙ]ₒ—, wherein m is an integer between 1 and 6, n is an integer between 0 and 3, o is an integer between 1 and 3, wherein if n is 0 then o is 1; Y is independently selected from 0, S, and —N(R¹³)—; and each of the C₁₋₆ alkylene, C₂₋₆ alkenylene, C₂₋₆ alkynylene, —(CH₂)ₘ—, and —(CH₂)ₙ— groups is optionally substituted with one or two independently selected R³⁰;
R⁶ is heteroaryl or heterocyclyl each of which is optionally substituted with one or more independently selected R⁷;
R⁷ is independently selected from the group consisting of alkyl, alkenyl, alkynyl, cycloalkyl, aryl, heterocyclyl, heteroaryl, halogen, —CN, azido, —NO₂, —OR¹¹, —N(R¹²)(R¹³), —N(R¹¹)(OR¹¹), —S(O)₀₋₂R¹¹, —S(O)₁₋₂OR¹¹, —OS(O)₁₋₂R¹¹, —OS(O)₁₋₂OR¹¹, —S(O)₁₋₂N(R¹²)(R¹³), —OS(O)₁₋₂N(R¹²)(R¹³), —N(R¹¹)S(O)₁₋₂R¹¹, —NR¹¹S(O)₁₋₂OR¹¹, —NR¹¹S(O)₁₋₂N(R¹²)(R¹³), —P(O)(OR¹¹)₂, —OP(O)(OR¹¹)₂, —C(═X)R¹¹, —C(═X)XR¹¹, —XC(═X)R¹¹, and —XC(═X)XR¹¹, and/or any two R⁷ which are bound to the same atom of R⁶ being a heterocyclyl group may join together to form ═O, wherein each of the alkyl, alkenyl, alkynyl, cycloalkyl, aryl, heterocyclyl, and heteroaryl groups is optionally substituted with one or more independently selected R³⁰;

### E is O or S;

R¹¹ is independently selected from the group consisting of H, alkyl, alkenyl, alkynyl, cycloalkyl, aryl, heteroaryl, and heterocyclyl, wherein each of the alkyl, alkenyl, alkynyl, cycloalkyl, aryl, heteroaryl, and heterocyclyl groups is optionally substituted with one or more independently selected R³⁰;
each of R¹² and R¹³ is independently selected from the group consisting of H, alkyl, alkenyl, alkynyl, cycloalkyl, aryl, heteroaryl, and heterocyclyl, or R¹² and R¹³ may join together with the nitrogen atom to which they are attached to form the group —N═CR¹⁵R¹⁶, wherein each of the alkyl, alkenyl, alkynyl, cycloalkyl, aryl, heteroaryl, and heterocyclyl groups is optionally substituted with one or more independently selected R³⁰;
each of R¹⁵ and R¹⁶ is independently selected from the group consisting of H, alkyl, alkenyl, alkynyl, cycloalkyl, aryl, heteroaryl, heterocyclyl, and —NH_{y}R²⁰_{2-y}, or R¹⁵ and R¹⁶ may join together with the atom to which they are attached to form a ring which is optionally substituted with one or more independently selected R³⁰, wherein each of the alkyl, alkenyl, alkynyl, cycloalkyl, aryl, heteroaryl, and heterocyclyl groups is optionally substituted with one or more independently selected R³⁰;
y is an integer from 0 to 2;
R²⁰ is independently selected from the group consisting of alkyl, alkenyl, alkynyl, cycloalkyl, aryl, heteroaryl, and heterocyclyl, wherein each of the alkyl, alkenyl, alkynyl, cycloalkyl, aryl, heteroaryl, and heterocyclyl groups is optionally substituted with one or more independently selected R³⁰; and
R³⁰ is a 1^{st} level substituent and is, in each case, independently selected from the group consisting of alkyl, alkenyl, alkynyl, aryl, heteroaryl, cycloalkyl, heterocyclyl, halogen, —CN, azido, —NO₂, —OR⁷¹, —N(R⁷²)(R⁷³), —S(O)₀₋₂R⁷¹, —S(O)₁₋₂OR⁷¹, —OS(O)₁₋₂R⁷¹, —OS(O)₁₋₂OR⁷¹, —S(O)₁₋₂N(R⁷²)(R⁷³), —OS(O)₁₋₂N(R⁷²)(R⁷³), —N(R⁷¹)S(O)₁₋₂R⁷¹, —NR⁷¹S(O)₁₋₂OR⁷¹, —NR⁷¹S(O)₁₋₂N(R⁷²)(R⁷³), —OP(O)(OR⁷¹)₂, —C(═X¹)R⁷¹, —C(═X¹)X¹R⁷¹, —X¹C(═X¹)R⁷¹, and —X¹C(═X¹)X¹R⁷¹, and/or any two R³⁰ which are bound to the same carbon atom of a cycloalkyl or heterocyclyl group may join together to form ═X¹, wherein each of the alkyl, alkenyl, alkynyl, aryl, heteroaryl, cycloalkyl, and heterocyclyl groups being a 1^{st} level substituent is optionally substituted by one or more 2^{nd} level substituents, wherein said 2^{nd} level substituent is, in each case, independently selected from the group consisting of C₁₋₆ alkyl, C₂₋₆ alkenyl, C₂₋₆ alkynyl, 3- to 14-membered aryl, 3- to 14-membered heteroaryl, 3- to 14-membered cycloalkyl, 3- to 14-membered heterocyclyl, halogen, —CF₃, —CN, azido, —NO₂, —OR⁸¹, —N(R⁸²)(R⁸³), —S(O)₀₋₂R⁸¹, —S(O)₁₋₂OR⁸¹, —OS(O)₁₋₂R⁸¹, —OS(O)₁₋₂OR⁸¹, —S(O)₁₋₂N(R⁸²)(R⁸³), —OS(O)—₂N(R⁸²)(R⁸³), —N(R⁸¹)S(O)₁₋₂R⁸¹, —NR⁸¹S(O)₁₋₂OR⁸¹, —NR⁸¹S(O)₁₋₂N(R⁸²)(R⁸³), —OP(O)(OR⁸¹)₂, —C(═X²)R⁸¹, —C(═X²)X²R⁸¹, —X²C(═X²)R⁸¹, and —X²C(═X²)X²R⁸¹, and/or any two 2^{nd} level substituents which are bound to the same carbon atom of a cycloalkyl or heterocyclyl group being a 1^{st} level substituent may join together to form ═X², wherein each of the C₁₋₆ alkyl, C₂₋₆ alkenyl, C₂₋₆ alkynyl, 3- to 14-membered aryl, 3- to 14-membered heteroaryl, 3- to 14-membered cycloalkyl, 3- to 14-membered heterocyclyl groups being a 2^{nd} level substituent is optionally substituted with one or more 3rd level substituents, wherein said 3^{rd} level substituent is, in each case, independently selected from the group consisting of C₁₋₃ alkyl, halogen, —CF₃, —CN, azido, —NO₂, —OH, —O(C₁₋₃ alkyl), —OCF₃, —S(C₁₋₃ alkyl), —NH₂, —NH(C₁₋₃ alkyl), —N(C₁₋₃ alkyl)₂, —NHS(O)₂(C₁₋₃ alkyl), —S(O)₂NH_{2-z}(C₁₋₃ alkyl)_{z}, —C(═O)OH, —C(═O)O(C₁₋₃ alkyl), —C(═O)NH_{2-z}(C₁₋₃ alkyl)_{z}, —NHC(═O)(C₁₋₃ alkyl), —NHC(═NH)NH_{z-2}(C₁₋₃ alkyl)_{z}, and —N(C₁₋₃ alkyl)C(═NH)NH_{2-z}(C₁₋₃ alkyl)_{z}, wherein each z is independently 0, 1, or 2 and each C₁₋₃ alkyl is independently methyl, ethyl, propyl or isopropyl, and/or any two 3^{rd} level substituents which are bound to the same carbon atom of a 3- to 14-membered cycloalkyl or heterocyclyl group being a 2^{nd} level substituent may join together to form ═O, ═S, ═NH, or ═N(C₁₋₃ alkyl);
wherein
each of R⁷¹, R⁷², and R⁷³ is independently selected from the group consisting of H, C₁₋₆ alkyl, C₂₋₆ alkenyl, C₂₋₆ alkynyl, 3- to 7-membered cycloalkyl, 5- or 6-membered aryl, 5- or 6-membered heteroaryl, and 3- to 7-membered heterocyclyl, wherein each of the C₁₋₆ alkyl, C₂₋₆ alkenyl, C₂₋₆ alkynyl, 3- to 7-membered cycloalkyl, 5- or 6-membered aryl, 5- or 6-membered heteroaryl, and 3- to 7-membered heterocyclyl groups is optionally substituted with one, two or three substituents independently selected from the group consisting of C₁₋₃ alkyl, halogen, —CF₃, —CN, azido, —NO₂, —OH, —O(C₁₋₃ alkyl), —OCF₃, ═O, —S(C₁₋₃ alkyl), —NH₂, —NH(C₁₋₃ alkyl), —N(C₁₋₃ alkyl)₂, —NHS(O)₂(C₁₋₃ alkyl), —S(O)₂NH_{2-z}(C₁₋₃ alkyl)_{z}, —C(═O)(C₁₋₃ alkyl), —C(═O)OH, —C(═O)O(C₁₋₃ alkyl), —C(═O)NH_{2-z}(C₁₋₃ alkyl)_{z}, —NHC(═O)(C₁₋₃ alkyl), —NHC(═NH)NH_{z-2}(C₁₋₃ alkyl)_{z}, and —N(C₁₋₃ alkyl)C(═NH)NH_{2-z}(C₁₋₃ alkyl)_{z}, wherein each z is independently 0, 1, or 2 and each C₁₋₃ alkyl is independently methyl, ethyl, propyl or isopropyl;
each of R⁸¹, R⁸², and R⁸3 is independently selected from the group consisting of H, C₁₋₄ alkyl, C₂₋₄ alkenyl, C₂₋₄ alkynyl, 3- to 6-membered cycloalkyl, 5- or 6-membered aryl, 5- or 6-membered heteroaryl, and 3- to 6-membered heterocyclyl, wherein each of the C₁₋₄ alkyl, C₂₋₄ alkenyl, C₂₋₄ alkynyl, 3- to 6-membered cycloalkyl, 5- or 6-membered aryl, 5- or 6-membered heteroaryl, and 3- to 6-membered heterocyclyl groups is optionally substituted with one, two or three substituents independently selected from the group consisting of C₁₋₃ alkyl, halogen, —CF₃, —CN, azido, —NO₂, —OH, —O(C₁₋₃ alkyl), —OCF₃, ═O, —S(C₁₋₃ alkyl), —NH₂, —NH(C₁₋₃ alkyl), —N(C₁₋₃ alkyl)₂, —NHS(O)₂(C₁₋₃ alkyl), —S(O)₂NH_{2-z}(C₁₋₃ alkyl)_{z}, —C(═O)(C₁₋₃ alkyl), —C(═O)OH, —C(═O)O(C₁₋₃ alkyl), —C(═O)NH_{2-z}(C₁₋₃ alkyl)_{z}, —NHC(═O)(C₁₋₃ alkyl), —NHC(═NH)NH_{z-2}(C₁₋₃ alkyl)_{z}, and —N(C₁₋₃ alkyl)C(═NH)NH_{2-z}(C₁₋₃ alkyl)_{z}, wherein each z is independently 0, 1, or 2 and each C₁₋₃ alkyl is independently methyl, ethyl, propyl or isopropyl; and
each of X¹ and X² is independently selected from O, S, and N(R⁸⁴), wherein R⁸⁴ is H or C₁₋₃ alkyl;
with the proviso that:
when E is O and either of (1) or (2) is true:
(3) R¹ and R³ do not join together via a group L′ to form a moiety R¹-L′-R³; or
(4) both of R^{4a} and R^{4b} are H,
then either:
(c) R⁶ is: (i) a 5-membered monocyclic heteroaryl which contains at least one S ring atom and which is substituted with one or more independently selected R⁷; (ii) a 5-membered monocyclic heteroaryl which contains at least two nitrogen atoms and which is substituted with one or more independently selected R⁷; or (iii) a 5-membered monocyclic heteroaryl which contains at least one nitrogen atom and at least one oxygen atom and which is substituted with one or more independently selected R⁷; or
(d) R⁷ is independently selected from the group consisting of alkyl, alkenyl, alkynyl, cycloalkyl, aryl, heterocyclyl, heteroaryl, halogen, —CN, azido, —NO₂, —OR¹¹, —N(R¹²)(R¹³), —N(R¹¹)(OR¹¹), —S(O)₀₋₂R¹¹, —S(O)₁₋₂OR¹¹, —OS(O)₁₋₂R¹¹, —OS(O)₁₋₂OR¹¹, —S(O)₁₋₂N(R¹²)(R¹³), —OS(O)₁₋₂N(R¹²)(R¹³), —N(R¹¹)S(O)₁₋₂R¹¹, —NR¹¹S(O)₁₋₂OR¹¹, —NR¹¹S(O)₁₋₂N(R¹²)(R¹³), —P(O)(OR¹¹)₂, —OP(O)(OR¹¹)₂, —C(═X)R¹¹, —C(═X)XR¹¹, —XC(═X)R¹¹, and —XC(═X)XR¹¹, wherein each of the alkyl, alkenyl, alkynyl, cycloalkyl, aryl, heterocyclyl, and heteroaryl groups is optionally substituted with one or more independently selected R³⁰, wherein at least one of R⁷ is F and/or at least one of R⁷ is substituted with one or more F atoms.
   ITEM 1a. The compound of item 1, wherein R⁶ is a 5-membered monocyclic heteroaryl which contains at least one S ring atom and which is substituted with one or more independently selected R⁷;

R⁷ is independently selected from the group consisting of alkyl, alkenyl, alkynyl, cycloalkyl, aryl, heterocyclyl, heteroaryl, halogen, —CN, azido, —NO₂, —OR¹¹, —N(R¹²)(R¹³), —N(R¹¹)(OR¹¹), —S(O)₀₋₂R¹¹, —S(O)₁₋₂OR¹¹, —OS(O)₁₋₂R¹¹, —OS(O)₁₋₂OR¹¹, —S(O)₁₋₂N(R¹²)(R¹³), —OS(O)₁₋₂N(R¹²)(R¹³), —N(R¹¹)S(O)₁₋₂R¹¹, —NR¹¹S(O)₁₋₂OR¹¹, —NR¹¹S(O)₁₋₂N(R¹²)(R¹³), —P(O)(OR¹¹)₂, —OP(O)(OR¹¹)₂, —C(═X)R¹¹, —C(═X)XR¹¹, —XC(═X)R¹¹, and —XC(═X)XR¹¹; and wherein each of the alkyl, alkenyl, alkynyl, cycloalkyl, aryl, heterocyclyl, and heteroaryl groups is optionally substituted with one or more independently selected R³⁰, and

wherein at least one of R⁷ is F and/or at least one of R⁷ is substituted with one or more F atoms.

ITEM 1b. The compound of item 1a, wherein at least one of R⁷ is F and/or at least one of R⁷ is C₁₋₃ alkyl, wherein the alkyl group of C₁₋₃ alkyl is substituted with one or more F atoms.
ITEM 2. The compound of any of items 1 to 1b, wherein R^{1a} is selected from the group consisting of alkyl, —O(alkyl), —S(alkyl), —NH(alkyl), —N(alkyl)₂, and heterocyclyl, wherein each of the alkyl and heterocyclyl groups is optionally substituted with one or more independently selected R³⁰.
ITEM 3. The compound of item 2, wherein the one or more independently selected R³⁰ optionally substituting R^{1a} are selected from (i) C₁₋₃ alkyl, phenyl, thiazolidinyl, halogen, —NH₂, —NHS(O)₂(C₁₋₃ alkyl), —NHC(═O)(C₁₋₃ alkyl), and —NHC(═NH)NH_{z-2}(C₁₋₃ alkyl)_{z}, wherein z is 0, 1, or 2 and each C₁₋₃ alkyl is independently methyl, ethyl, propyl or isopropyl; (ii) methyl, ethyl, propyl, isopropyl, phenyl, ═O, and ═S; or (iii) methyl, ethyl, propyl, isopropyl, halogen, and —CF₃.
ITEM 4. The compound of any of the items 1 to 3, wherein R^{1a} is selected from the group consisting of —O(alkyl), —S(alkyl), —NH(alkyl), —N(alkyl)₂, and heterocyclyl, wherein each of the alkyl and heterocyclyl groups is optionally substituted with one or more independently selected R³⁰,
ITEM 5. The compound of any of the items 1 to 4, wherein R^{1a} is selected from the group consisting of C₁₋₃ alkyl, —O(C₁₋₃ alkyl), —S(C₁₋₃ alkyl), —NH(C₁₋₃ alkyl), piperazinyl, morpholinyl, piperidinyl, pyrrolidinyl, and azepanyl wherein each of the piperazinyl, morpholinyl, piperidinyl, pyrrolidinyl, and azepanyl groups is optionally substituted with one or two moieties independently selected from the group consisting of methyl, ethyl, —OH, —OCH₃, —SCH₃, cyclopropyl, 2-hydroxyethyl, 2-(N,N-dimethylamino)ethyl, 2-(N,N-dimethylamino)ethoxy, 2-aminoethyl, 2-(N-methylamino)ethyl, 2-(methoxy)ethyl, 4-methylpiperazinyl, —C(═O)(C₁₋₃ alkyl), —(CH₂)₁₋₃COOH, and —NH_{2-z}(CH₃)_{z}, wherein z is 0, 1, or 2; and each of the C₁₋₃ alkyl groups is optionally substituted with one or two moieties independently selected from the group consisting of —OH, —OCH₃, —SCH₃, cyclopropyl, piperazinyl, 4-methyl-piperazinyl, 4-(2-hydroxyethyl)piperazinyl, 2-(N,N-dimethylamino)ethoxy, and —NH_{2-z}(CH₃)_{z}, wherein z is 0, 1, or 2.
ITEM 6. The compound of any of the items 1 to 5, wherein R^{1a} is selected from the group consisting of —NH(C₁₋₃ alkyl), piperazinyl, piperidinyl, pyrrolidinyl, and azepanyl wherein the piperazinyl group is optionally substituted with one or two moieties independently selected from the group consisting of 2-hydroxyethyl, methyl, —CH₂COOH, and —C(═O)CH₃; the piperidinyl group is optionally substituted with one to three R³⁰ independently selected from the group consisting of methyl, —NH₂ and 4-methylpiperazinyl; the pyrrolidinyl is optionally substituted with one or two R³⁰ being —OH; and each of the C₁₋₃ alkyl groups is optionally substituted with one or two R³⁰ independently selected from the group consisting of —OH, —OCH₃, and —NH_{2-z}(CH₃)_{z}, wherein z is 0, 1, or 2.
ITEM 7. The compound of any of the items 1 to 6, wherein R^{1a} is selected from the group consisting of piperidinyl substituted with one to three moieties independently selected from the group consisting of C₁ to C₄ alkyl; piperazinyl group substituted with one or two R³⁰ independently selected from the group consisting of 2-hydroxyethyl and C₁ to C₄ alkyl; azepanyl substituted with one to three R³⁰ independently selected from the group consisting of C₁ to C₄ alkyl; morpholinyl; and C₁₋₃ alkyl group substituted with R³⁰ being —NH_{2-z}(CH₃)_{z}, wherein z is 0, 1, or 2.
ITEM 8. The compound of any of the items 1 to 7, wherein R^{1a} is piperidinyl substituted with one to three R³⁰ independently selected from the group consisting of C₁ to C₄ alkyl.
ITEM 9. The compound of any of the items 1 to 8, wherein R^{1a} is piperidinyl substituted with one to three R³⁰ methyl.
ITEM 10. The compound of any of the items 1 to 9, wherein R^{1a} is selected from the group consisting of 1-methylpiperidinyl, 1,2-dimethylpiperidinyl, 1,2,6-trimethylpiperidinyl, 1-methylazepanyl, 4-(2-hydroxyethyl)piperazinyl, 1-methyl-4-(2-hydroxyethyl)piperazinyl, 4-methylpiperazinyl, 4-acetylpiperazinyl, and (2-hydroxyethyl)amino.
ITEM 11. The compound of any of the items 1 to 10, wherein Q is selected from the group consisting of a 3-to 10-membered mono or bicyclic cycloalkyl, aryl, heterocyclyl, heteroaryl, wherein each of the cycloalkyl, aryl, heterocyclyl, and heteroaryl groups is optionally substituted with one or more independently selected R³⁰,
ITEM 12. The compound of any of the items 1 to 11, wherein Q is selected from the group consisting of 3- to 10-membered mono or bicyclic cycloalkyl, aryl, and heteroaryl, wherein each of the cycloalky, aryl and heteroaryl groups is optionally substituted with one or more independently selected R³⁰.
ITEM 13. The compound of any of the items 1 to 12, wherein Q is selected from the group consisting of C₆₋₁₀ aryl, and 5- or 6-membered heteroaryl, wherein each of the C₆₋₁₀ aryl, and 5- or 6-membered heteroaryl is optionally substituted with one or more R³⁰ independently selected from the group consisting of C₁₋₈ alkyl, halogen, —OR¹¹, —N(R¹²)(R¹³), —SR¹¹, C(═O)R¹¹, wherein each of the C₁₋₈ alkyl, halogen, —OR¹¹, —N(R¹²)(R¹³), —SR¹¹, C(═O)R¹¹ is optionally substituted with one or more independently selected R³⁰.
ITEM 14. The compound of any of the items 1 to 13, wherein Q is C₆₋₁₀ aryl, wherein the C₆₋₁₀ aryl is optionally substituted with one or more R³⁰ being —OR¹¹, wherein R¹¹ is independently selected from C₁₋₁₂alkyl.
ITEM 15. The compound of any of the items 1 to 14, wherein Q is C₆₋₁₀ aryl, wherein the C₆₋₁₀ aryl is optionally substituted with one or more R³⁰ being —OR¹¹, wherein R¹¹ is independently selected from C₁₋₈ alkyl.
ITEM 16. The compound of any of the items 1 to 15, wherein Q is C₆₋₁₀ aryl, wherein the C₆₋₁₀ aryl is optionally substituted with one or more R³⁰ being —OR¹¹, wherein R¹¹ is independently selected from C₁₋₄ alkyl.
ITEM 17. The compound of any of the items 1 to 16, wherein Q is C₆ aryl, wherein the C₆ aryl is optionally substituted with one or more R³⁰ being —OR¹¹, wherein R¹¹ is independently selected from C₁₋₄ alkyl.
ITEM 18. The compound of any of the items 1 to 17, wherein Q is C₆ aryl, wherein the C₆ aryl is optionally substituted with one or more R³⁰ being —OR¹¹, wherein R¹¹ is methyl.
ITEM 19. The compound of any of the items 14 to 18, wherein R^{1a} and —OR¹¹ are in meta-position with respect to each other.
ITEM 20. The compound of any of the items 14 to 19, wherein R^{1a} and the bond between R¹ and N in Formula (I) are in meta-position or in para-position with respect to each other.
ITEM 21. The compound of any of the items 14 to 20, wherein R^{1a} and the bond between R¹ and N in Formula (I) are in para-position with respect to each other.
ITEM 22. The compound of any of the items 14 to 21, wherein —OR¹¹ and the bond between R¹ and N in Formula (I) are in ortho-position with respect to each other.
ITEM 22a. The compound of any of the items 1 to 13, wherein Q is C₆₋₁₀ aryl; R¹ and R³ join together via a group L′ to form a moiety Q-L′-R³; and the bond between Q and L′ and R¹, are in ortho-position with respect to each other.
ITEM 23. The compound of any of the items 1 to 22a, wherein E is O.
ITEM 24. The compound of any of the items 1 to 23, wherein R³ is selected from the group consisting of H, alkyl, alkenyl, alkynyl, cycloalkyl, aryl, heterocyclyl, heteroaryl, wherein each of the alkyl, alkenyl, alkynyl, cycloalkyl, aryl, heterocyclyl, and heteroaryl groups is optionally substituted with one or more independently selected R³⁰,
ITEM 25. The compound of any of the items 1 to 24, wherein R³ is selected from the group consisting of alkyl, cycloalkyl, aryl, and heteroaryl, wherein each of the alkyl, cycloalkyl, aryl, and heteroaryl groups is optionally substituted with one or more independently selected R³⁰.
ITEM 26. The compound of any of the items 1 to 25, wherein R³ is selected from the group consisting of C₁₋₈ alkyl, C₃₋₁₀ cycloalkyl, C₆₋₁₀ aryl, and 5- to 8-membered heteroaryl, wherein each of the C₁₋₈ alkyl, C₃₋₁₀ cycloalkyl, C₆₋₁₀ aryl, and 5- to 8-membered heteroaryl groups is optionally substituted with one or more independently selected R³⁰.
ITEM 27. The compound of any of the items 1 to 26, wherein R³ is selected from the group consisting of C₁₋₈ alkyl, C₃₋₁₀ cycloalkyl, C₆₋₁₀ aryl, and 5- to 8-membered heteroaryl, wherein each of the C₁₋₈ alkyl, C₃₋₁₀ cycloalkyl, C₆₋₁₀ aryl, and 5- to 8-membered heteroaryl groups is optionally substituted with one or more R³⁰ independently selected from the group consisting of C₁₋₈ alkyl, halogen, —OR¹¹, —N(R¹²)(R¹³), —SR¹¹, C(═O)R¹¹, wherein each of C₁₋₈ alkyl, halogen, —OR¹¹, —N(R¹²)(R¹³), —SR¹¹, and C(═O)R¹¹ is optionally substituted with one or more independently selected R³⁰.
ITEM 28. The compound of any of the items 1 to 27, wherein R³ is selected from the group consisting of C₁₋₈ alkyl, C₃₋₁₀ cycloalkyl, C₆₋₁₀ aryl, and 5- to 8-membered heteroaryl, wherein each of the C₁₋₈ alkyl, C₃₋₁₀ cycloalkyl, C₆₋₁₀ aryl, and 5- to 8-membered heteroaryl groups is optionally substituted with one or more R³⁰ independently selected from the group consisting of C₁₋₈ alkyl, halogen, —OR¹¹, —N(R¹²)(R¹³), —SR¹¹, and C(═O)R¹¹.
ITEM 29. The compound of any of the items 1 to 28, wherein R³ is selected from the group consisting of C₁₋₆ alkyl, C₃₋₆ cycloalkyl, C₆ aryl, and 5- or 6-membered heteroaryl, wherein each of the C₁₋₆ alkyl, C₃₋₆ cycloalkyl, C₆ aryl, and 5- or 6-membered heteroaryl groups is optionally substituted with one or more R³⁰ independently selected from the group consisting of C₁₋₆ alkyl, and OR¹¹, wherein R¹¹ C₁₋₁₂ alkyl.
ITEM 30. The compound of any of the items 1 to 29, wherein R³ is selected from the group consisting of C₁₋₄ alkyl, C₆ aryl, and 6-membered N-containing heteroaryl, wherein each of the C₁₋₄ alkyl, C₆ aryl, and 6-membered heteroaryl groups is optionally substituted with one or more R³⁰ independently selected from the group consisting of C₁₋₄ alkyl, and OR¹¹, wherein R¹¹ C₁₋₄ alkyl.
ITEM 31. The compound of any of the items 1 to 30, wherein R³ is selected from the group consisting of methyl, ethyl, phenyl, and pyridyl, wherein each of the methyl, ethyl, phenyl, and pyridyl is optionally substituted with one or more R³⁰ being methoxy.
ITEM 32. The compound of any of the items 1 to 31, wherein R³ is selected from the group consisting of methyl, 2-methoxyethyl, methoxyphenyl, and 3-methoxypyridyl, and 1,3-dimethoxyphenyl.
ITEM 33. The compound of any of the items 1 to 32, wherein R³ is methoxypyridyl.
ITEM 34. The compound of any of the items 1 to 31, wherein R¹ and R³ join together via a group L′ to form a moiety R¹-L′-R³ and, optionally, wherein R³ is a bond.
ITEM 34a. The compound of item 34, wherein R¹ and R³ join together via a group L′ to form a moiety Q-L′-R³, and optionally, wherein R³ is a bond.
ITEM 35. The compound of any of the items 1 to 34a, wherein R^{4a} and R^{4b} are independently selected from the group consisting of H, C₁₋₃ alkyl, and C₂₋₃ alkyloxy; or optionally R^{4a} and R^{4b} may join together to form, together with the carbon to which they are attached, C₃₋₆ cycloalkyl or a 4- to 6-membered heterocyclyl comprising at least one O as the only heteroatom element, wherein in case that the 4- to 6-membered heterocyclyl comprises more than one O, different O are not directly bound to each other.
ITEM 36. The compound of any of the items 1 to 35, wherein R^{4a} and R^{4b} are independently selected from the group consisting of H, C₁ or C₂ alkyl; or optionally R^{4a} and R^{4b} may join together to form, together with the carbon to which they are attached, C₃ or C₄ cycloalkyl or a 4-membered heterocyclyl comprising one O as the only heteroatom element.
ITEM 37. The compound of any of the items 1 to 36, wherein R^{4a} and R^{4b} are independently selected from the group consisting of H, C₁ or C₂ alkyl; or optionally R^{4a} and R^{4b} may join together to form, together with the carbon to which they are attached, C₃ cycloalkyl.
ITEM 38. The compound of any of the items 1 to 37, wherein R^{4a} and R^{4b} are independently selected from the group consisting of H, and methyl; or optionally R^{4a} and R^{4b} may join together to form, together with the carbon to which they are attached, cyclopropyl.
ITEM 39. The compound of any of the items 1 to 38, wherein both R^{4a} and R^{4b} are H.
ITEM 40. The compound of any of the items 1 and 2 to 39, wherein R⁶ is a mono- or bicyclic heteroaryl or a mono- or bicyclic heterocyclyl, each of which is optionally substituted with one or more independently selected R⁷.
ITEM 41. The compound of any of the items 1 and 2 to 40, wherein R⁶ is a 5- to 6-membered monocyclic heteroaryl optionally substituted with one, two, three or four independently selected R⁷.
ITEM 42. The compound of any of the items 1 and 2 to 41, wherein R⁶ is a 5-membered monocyclic heteroaryl which contains at least one ring heteroatom selected from the group consisting of N, O, and S and which is optionally substituted with one, two, or three independently selected R⁷.
ITEM 43. The compound of any of the items 1 and 2 to 41, wherein R⁶ is a 5- or 6-membered monocyclic heteroaryl which contains at least one S ring atom and which is optionally substituted with one, two, or three independently selected R7.
ITEM 44. The compound of any of the items 1 to 43, wherein R⁶ is thienyl optionally substituted with one, two, or three independently selected R⁷.
ITEM 45. The compound of any of the items 1 to 44, wherein R⁶ is substituted with one, two, or three independently selected R⁷.
ITEM 45a. The compound of item 45, wherein R⁶ is substituted with two independently selected R⁷, optionally wherein R⁶ is substituted with two R⁷ that differ from each other.
ITEM 46. The compound of any of the items 1 to 45a, wherein R⁷ is independently selected from the group consisting of C₁₋₃ alkyl, halogen, —CN, —O(C₁₋₃ alkyl), —NH(C₁₋₃ alkyl), and —N(C₁₋₃ alkyl)₂, and/or any two R⁷ which are bound to the same atom of R⁶ being a heterocyclyl group may join together to form ═O, wherein each of the C₁₋₃ alkyl groups is optionally substituted with one or more independently selected R³⁰.
ITEM 47. The compound of any of the items 1 to 46, wherein R⁷ is independently selected from the group consisting of halogen and C₁₋₂ alkyl, wherein the C₁₋₂ alkyl groups is optionally substituted with one, two, or three independently selected R³⁰.
ITEM 48. The compound of any of the items 1 to 47, wherein R⁷ is independently selected from the group consisting of Cl, F, methyl, fluoromethyl, difluoromethyl, and trifluoromethyl.
ITEM 49. The compound of any of the items 1 to 48, wherein one R⁷ group is bound to a ring atom of R⁶ at position 2 relative to the ring atom by which R⁶ is bound to the remainder of the compound.
ITEM 50. The compound of any of the items 1 to 49, wherein one R⁷ group is bound to a ring atom of R⁶ at position 5 relative to the ring atom by which R⁶ is bound to the remainder of the compound.
ITEM 51. The compound of any of the items 1 to 50, wherein R⁶ is substituted with two, or three independently selected R⁷, and one R⁷ group is bound to a ring atom of R⁶ at position 2 relative to the ring atom by which R⁶ is bound to the remainder of the compound, and another R⁷ group is bound to a ring atom of R⁶ at position 5 relative to the ring atom by which R⁶ is bound to the remainder of the compound.
ITEM 52. The compound of any of the items 1 to 42, wherein R⁶ is selected from the group consisting of

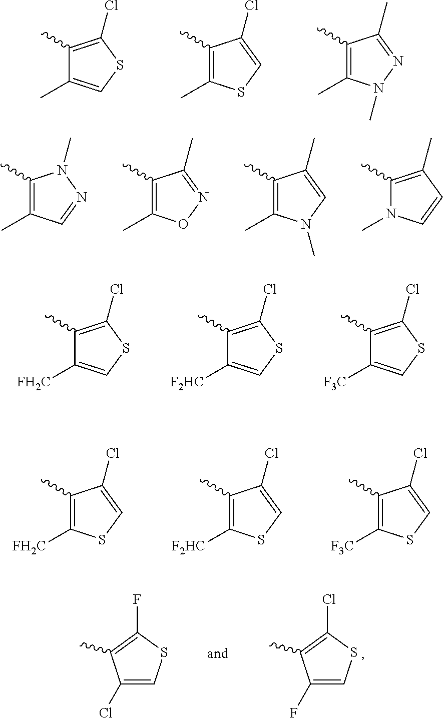

wherein [symbol] represents the bond by which R⁶ is bound to the remainder of the compound.
ITEM 53. The compound of any of the items 1 to 42 or 52, wherein R⁶ is selected from the group consisting of

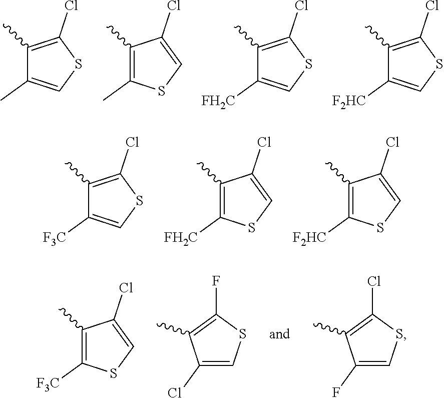

wherein [symbol] represents the bond by which R⁶ is bound to the remainder of the compound.
ITEM 53a. The compound of item 53, wherein R⁶ is selected from the group consisting of

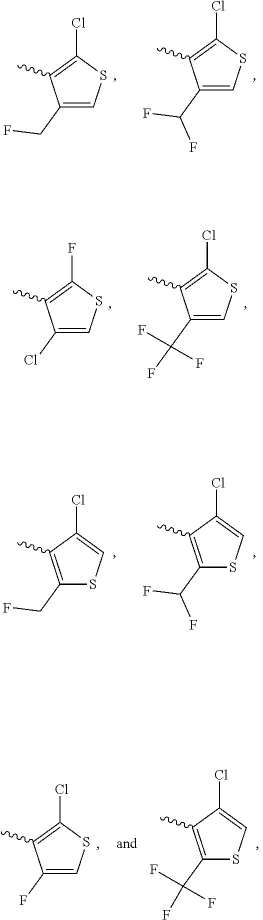

wherein [symbol] represents the bond by which R⁶ is bound to the remainder of the compound.
ITEM 54. The compound of any of the items 1 to 53a, wherein L is selected from the group consisting of a bond; C₁ alkylene, optionally substituted with one R³⁰; C₂ alkylene (in particular 1,2-ethylene or 1,1-ethylene), optionally substituted with one R³⁰; C₃ alkylene (in particular trimethylene), optionally substituted with one R³⁰; C₄ alkylene (in particular tetramethylene or 2,4-butandiyl), optionally substituted with one R³⁰; —(CH₂)ₘO—; and —(CH₂)ₘNH—, wherein m is 1, 2, or 3.
ITEM 55. The compound of any of the items 1 to 54, wherein L is a bond.
ITEM 56. The compound of any of the items 1 to 55, wherein R¹ and R³ join together via a group L′ to form a moiety R¹-L′-R³ and wherein L′ is selected from the group consisting of C₃₋₁₀ alkylene, C₃₋₁₀ alkenylene, C₃₋₁₀ alkynylene, —(CH₂)ₚ—[Y—(CH₂)_{q}]ᵣ—, and alkenylene —[Y—(CH₂)_{q}]ᵣ—, wherein p is an integer between 1 and 10, q is an integer between 0 and 6, r is an integer between 1 and 3, wherein if q is 0 then r is 1; Y is independently selected from 0, S, and —N(R¹³)—; and each of the C₃₋₁₀ alkylene, C₃₋₁₀ alkenylene, C₃₋₁₀ alkynylene, —(CH₂)ₚ—, and —(CH₂)_{q}— groups is optionally substituted with one or more independently selected R³⁰; optionally wherein R¹ and R³ join together via a group L′ to form a moiety Q-L′-R³.
ITEM 57. The compound of item 56, wherein L′ is selected from the group consisting of C₃₋₁₀ alkenylene, and C₃₋₁₀ alkenylene —[Y—(CH₂)_{q}]ᵣ—.
ITEM 58. The compound of item 56 or 57, wherein L′ is selected from the group consisting of C₃₋₁₀ alkenylene, and C₃₋₁₀ alkenylene —[O—(CH₂)_{q}]ᵣ—.
ITEM 59. The compound of any of the items 56 to 58, wherein L′ is C₃₋₁₀ alkenylene-[O—(CH₂)_{q}]ᵣ—.
ITEM 60. The compound of any of the items 56 to 59, wherein L′ is C₃₋₁₀ alkenylene-O—.
ITEM 61. The compound of any of the items 56 to 60, wherein L′ is C₄₋₈ alkenylene-O—.
ITEM 62. The compound of any of the items 56 to 61, wherein L′ is selected from the group consisting of

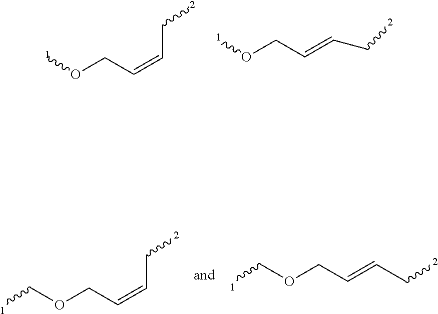

wherein [symbol] represents the bond by which L′ is bound to R¹, and [symbol] 2 represents the bond by which L′ is bound to R³.
ITEM 62a. The compound of any of the items 56 to 62, wherein R³ is a bond.
ITEM 63. The compound of item 1, wherein:
R^{1a} is selected from the group consisting of alkyl, —O(alkyl), —S(alkyl), —NH(alkyl), —N(alkyl)₂, and heterocyclyl, wherein each of the alkyl and heterocyclyl groups is optionally substituted with one or more independently selected R³⁰;
Q is selected from the group consisting of cycloalkyl, aryl, heterocyclyl, heteroaryl, wherein each of the cycloalkyl, aryl, heterocyclyl, and heteroaryl groups is optionally substituted with one or more independently selected R³⁰;
E is O;
R³ is selected from the group consisting of H, alkyl, alkenyl, alkynyl, cycloalkyl, aryl, heterocyclyl, heteroaryl, wherein each of the alkyl, alkenyl, alkynyl, cycloalkyl, aryl, heterocyclyl, and heteroaryl groups is optionally substituted with one or more independently selected R³⁰;
R^{4a} and R^{4b} are independently selected from the group consisting of H, C₁₋₃ alkyl; or optionally R^{4a} and R^{4b} may join together to form, together with the carbon to which they are attached, C₃₋₆ cycloalkyl or a 4- to 6-membered heterocyclyl comprising at least one O as the only heteroatom element, wherein in case that the 4- to 6-membered heterocyclyl comprises more than one O, different O are not directly bound to each other, preferably wherein R^{4a} and R^{4b} are independently selected from the group consisting of H, and methyl; or optionally R^{4a} and R^{4b} may join together to form, together with the carbon to which they are attached, cyclopropyl, most preferably wherein both R^{4a} and R^{4b} are H;
R⁶ is a mono- or bicyclic heteroaryl or a mono- or bicyclic heterocyclyl, each of which is optionally substituted with one, two, or three independently selected R⁷, preferably wherein R⁶ is a 5-membered monocyclic heteroaryl which contains at least one S ring atom and which is substituted with one or more independently selected R⁷;
R⁷ is independently selected from the group consisting of C₁₋₃ alkyl, halogen, —CN, —O(C₁₋₃ alkyl), —NH(C₁₋₃ alkyl), and —N(C₁₋₃ alkyl)₂, and/or any two R⁷ which are bound to the same atom of R⁶ being a heterocyclyl group may join together to form ═O, wherein each of the C₁₋₃ alkyl groups is optionally substituted with one or more independently selected R³⁰;
L is a bond; and
L′, if present, is selected from the group consisting of C₃₋₁₀ alkylene, C₃₋₁₀ alkenylene, C₃₋₁₀ alkynylene, —(CH₂)ₚ—[Y—(CH₂)_{q}]ᵣ—, and alkenylene —[Y—(CH₂)_{q}]ᵣ—, wherein p is an integer between 1 and 10, q is an integer between 0 and 6, r is an integer between 1 and 3, wherein if q is 0 then r is 1; Y is independently selected from 0, S, and —N(R¹³)—; and each of the C₃₋₁₀ alkylene, C₃₋₁₀ alkenylene, C₃₋₁₀ alkynylene, —(CH₂)ₚ—, and —(CH₂)_{q}— groups is optionally substituted with one or more independently selected R³⁰,
   ITEM 64. The compound of item 1, wherein:
R^{1a} is selected from the group consisting of piperidinyl substituted with one to three moieties independently selected from the group consisting of C₁ to C₄ alkyl; piperazinyl group substituted with one or two moieties independently selected from the group consisting of 2-hydroxyethyl and C₁ to C₄ alkyl; azepanyl substituted with one to three moieties independently selected from the group consisting of C₁ to C₄ alkyl; morpholinyl; and C₁₋₃ alkyl group substituted with —NH_{2-z}(CH₃)_{z}, wherein z is 0, 1, or 2;
Q is C₆₋₁₀ aryl, wherein the C₆₋₁₀ aryl is optionally substituted with one or more R³⁰ being —OR¹¹, wherein R¹¹ is independently selected from C₁₋₁₂ alkyl;
E is O;
R³ is selected from the group consisting of C₁₋₆ alkyl, C₆ aryl, and 5- or 6-membered heteroaryl, wherein each of the C₁₋₆ alkyl, C₆ aryl, and 5- or 6-membered heteroaryl groups is optionally substituted with one or more R³⁰ independently selected from the group consisting of C₁₋₆ alkyl, and OR¹¹, wherein R¹¹ C₁₋₁₂ alkyl;
R^{4a} and R^{4b} are independently selected from the group consisting of H, and methyl; or optionally R^{4a} and R^{4b} may join together to form, together with the carbon to which they are attached, cyclopropyl, preferably wherein both R^{4a} and R^{4b} are H;
R⁶ is thienyl substituted with one, two, or three independently selected R⁷;
R⁷ is independently selected from the group consisting of Cl, F, methyl, fluoromethyl, difluoromethyl, and trifluoromethyl, preferably wherein at least one of R⁷ is F and/or at least one of R⁷ is substituted with one or more F atoms;
L is a bond; and
L′, if present, is C₃₋₁₀ alkenylene-[O—(CH₂)_{q}]ᵣ—.
   ITEM 64a. The compound of any of the item 1, wherein:
R^{1a} is selected from the group consisting of alkyl, —O(alkyl), —S(alkyl), —NH(alkyl), —N(alkyl)₂, and heterocyclyl, wherein each of the alkyl and heterocyclyl groups is optionally substituted with one or more independently selected R³⁰;
Q is selected from the group consisting of cycloalkyl, aryl, heterocyclyl, heteroaryl, wherein each of the cycloalkyl, aryl, heterocyclyl, and heteroaryl groups is optionally substituted with one or more independently selected R³⁰;
E is O;
R³ is selected from the group consisting of H, alkyl, alkenyl, alkynyl, cycloalkyl, aryl, heterocyclyl, heteroaryl, wherein each of the alkyl, alkenyl, alkynyl, cycloalkyl, aryl, heterocyclyl, and heteroaryl groups is optionally substituted with one or more independently selected R³⁰;
both R^{4a} and R^{4b} are H;
R⁶ is selected from the group consisting of

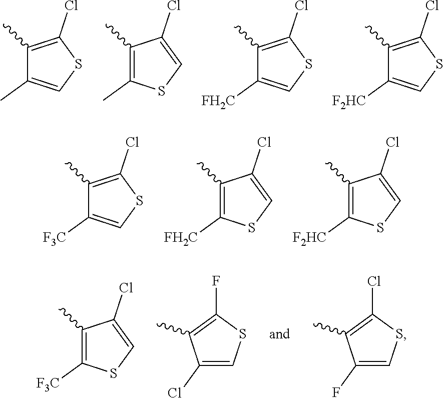

wherein [symbol] represents the bond by which R⁶ is bound to the remainder of the compound;
L is a bond; and
L′, if present, is selected from the group consisting of C₃₋₁₀ alkylene, C₃₋₁₀ alkenylene, C₃₋₁₀ alkynylene, —(CH₂)ₚ—[Y—(CH₂)_{q}]ᵣ, and alkenylene —[Y—(CH₂)_{q}]ᵣ—, wherein p is an integer between 1 and 10, q is an integer between 0 and 6, r is an integer between 1 and 3, wherein if q is 0 then r is 1; Y is independently selected from 0, S, and —N(R¹³)—; and each of the C₃₋₁₀ alkylene, C₃₋₁₀ alkenylene, C₃₋₁₀ alkynylene, —(CH₂)ₚ—, and —(CH₂)_{q}— groups is optionally substituted with one or more independently selected R³⁰,
   ITEM 65. The compound of item 1, wherein:
R^{1a} is selected from the group consisting of 1-methylpiperidinyl, 1,2-dimethylpiperidinyl, 1,2,6-trimethylpiperidinyl, 1-methylazepanyl, 4-(2-hydroxyethyl)piperazinyl, 1-methyl-4-(2-hydroxyethyl)piperazinyl, 4-methylpiperazinyl, 4-acetylpiperazinyl, and (2-hydroxyethyl)amino;
Q is C₆ aryl, wherein the C₆ aryl is optionally substituted with one or more —OR¹¹, wherein R¹¹ is methyl;
E is O;
R³ is selected from the group consisting of methyl, 2-methoxyethyl, methoxyphenyl, and 3-methoxypyridyl, and 1,3-dimethoxyphenyl;
both R^{4a} and R^{4b} are H;
R⁶ is selected from the group consisting of

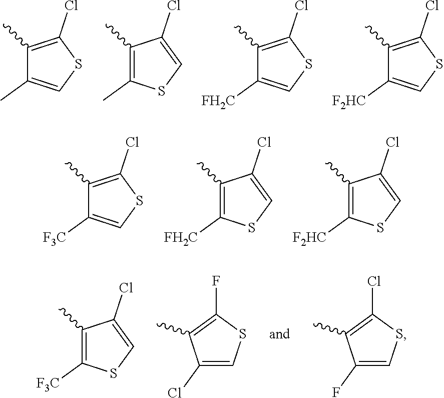

wherein [symbol] represents the bond by which R⁶ is bound to the remainder of the compound;
L is a bond; and
L′, if present, is selected from the group consisting of

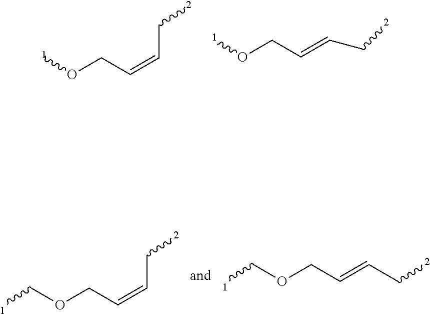

wherein 1 [symbol] represents the bond by which L′ is bound to R¹, and [symbol] 2 represents the bond by which L′ is bound to R³.
ITEM 65a. The compound of any one of items 63 to 65, wherein R6 is selected from the group consisting of:

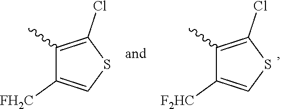

wherein [symbol] represents the bond by which R⁶ is bound to the remainder of the compound.
ITEM 65b. The compound of any one of items 63 to 65a, wherein R¹ and R³ join together via a group L′ to form a moiety R¹-L′-R³, preferably to form a moiety Q-L′-R³, and optionally wherein R³ is a bond.
ITEM 66. The compound of item 1, selected from the group consisting of

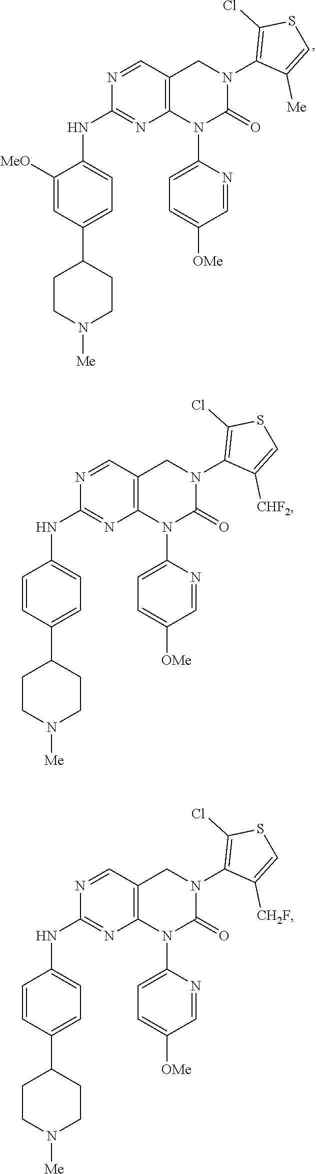

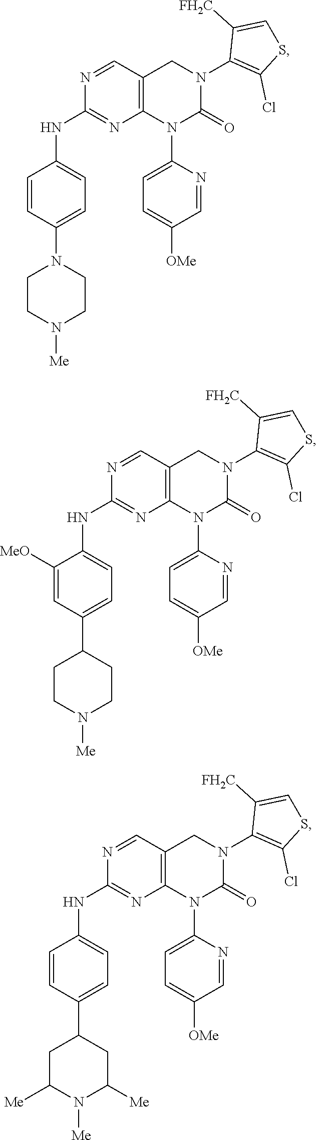

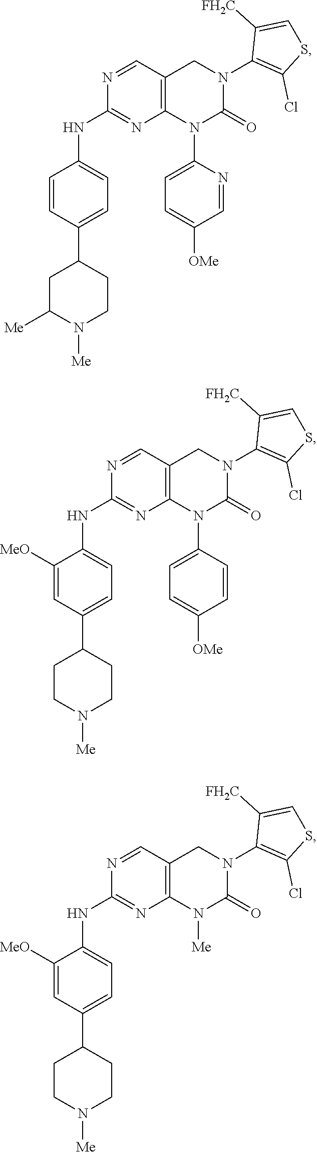

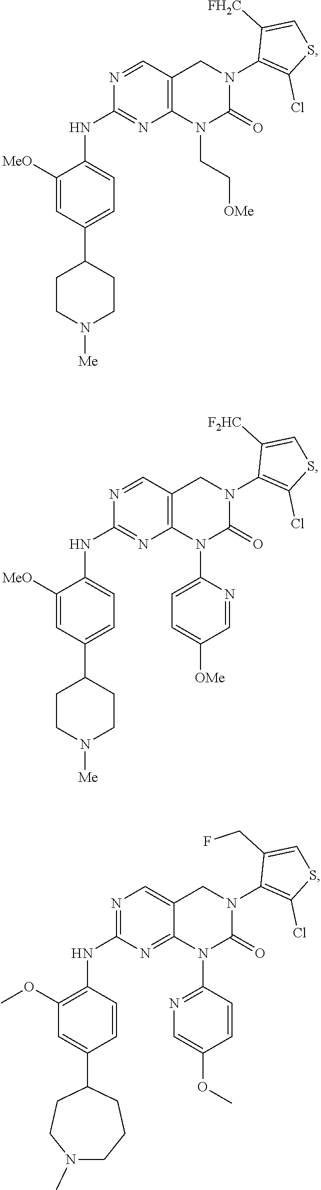

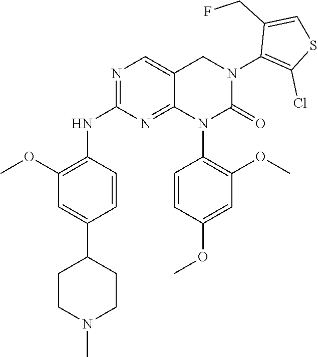 [SMILES: COc1ccc(N2C(=O)N(c3c(CF)csc3Cl)Cc3cnc(Nc4ccc(C5CCN(C)CC5)cc4OC)nc32)c(OC)c1]

and solvates, salts, N-oxides, complexes, polymorphs, crystalline forms, racemic mixtures, diastereomers, enantiomers, tautomers, conformers, isotopically labeled forms, prodrugs, and combinations thereof.
ITEM 66a. The compound of item 1, selected from the group consisting of

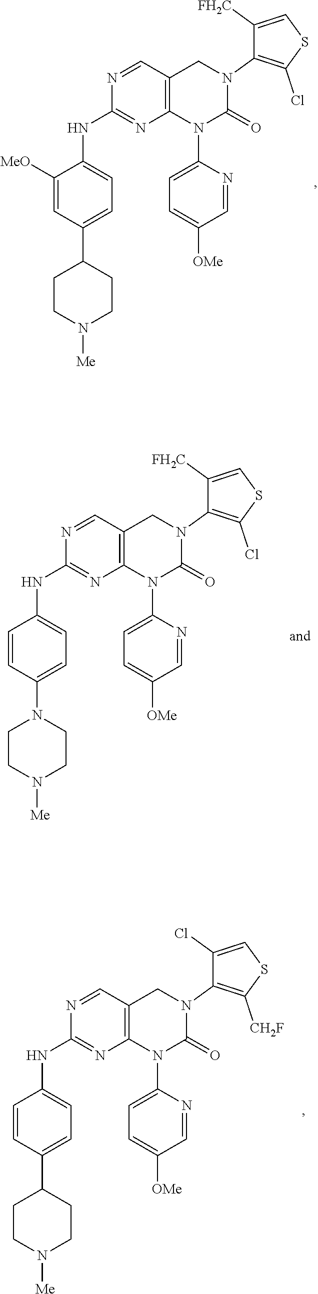

and solvates, salts, N-oxides, complexes, polymorphs, crystalline forms, tautomers, conformers, isotopically labeled forms, prodrugs, and combinations thereof.
ITEM 67. The compound of item 1, selected from the group consisting of

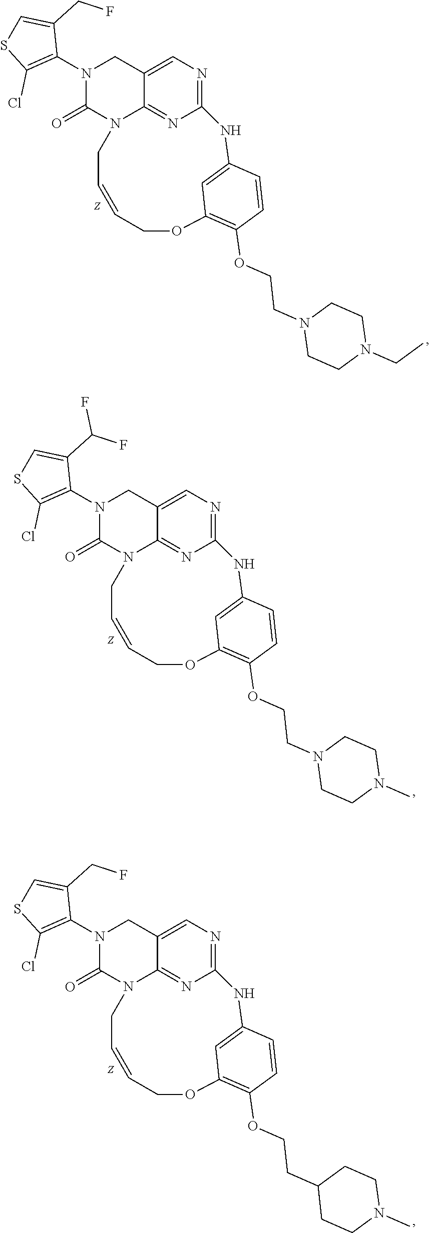 [SMILES: CCN1CCN(CCOc2ccc3cc2OC/C=C\CN2C(=O)N(c4c(CF)csc4Cl)Cc4cnc(nc42)N3)CC1 ; CN1CCN(CCOc2ccc3cc2OC/C=C\CN2C(=O)N(c4c(C(F)F)csc4Cl)Cc4cnc(nc42)N3)CC1 ; CN1CCC(CCOc2ccc3cc2OC/C=C\CN2C(=O)N(c4c(CF)csc4Cl)Cc4cnc(nc42)N3)CC1]

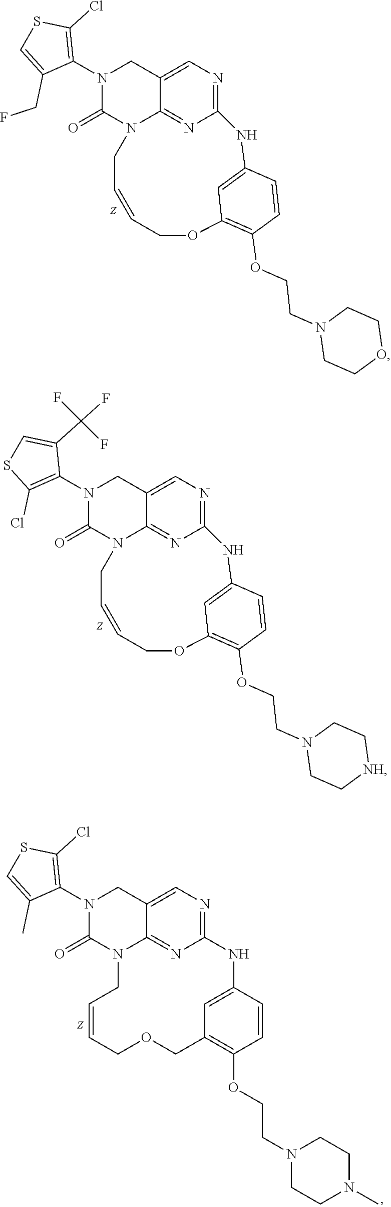 [SMILES: O=C1N2C/C=C\COc3cc(ccc3OCCN3CCOCC3)Nc3ncc(c2n3)CN1c1c(CF)csc1Cl ; O=C1N2C/C=C\COc3cc(ccc3OCCN3CCNCC3)Nc3ncc(c2n3)CN1c1c(C(F)(F)F)csc1Cl ; Cc1csc(Cl)c1N1Cc2cnc3nc2N(C/C=C\COCc2cc(ccc2OCCN2CCN(C)CC2)N3)C1=O]

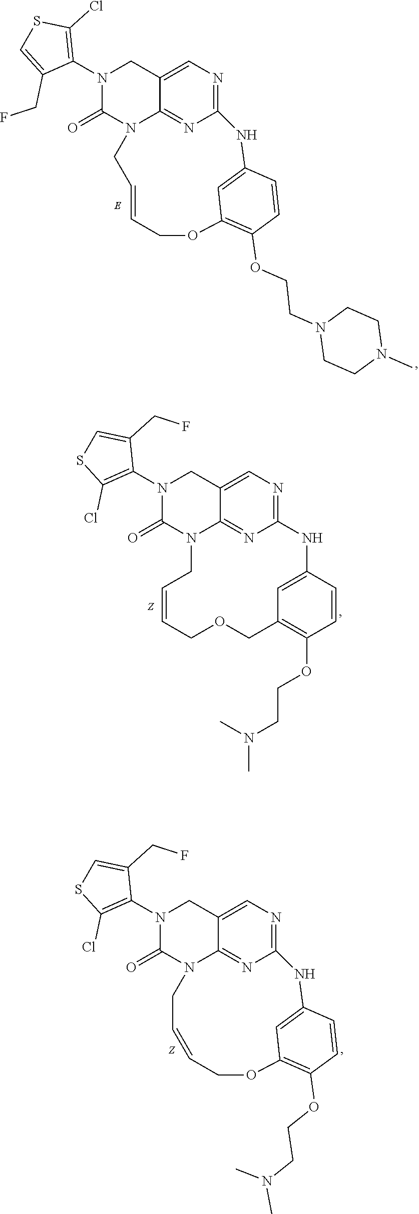 [SMILES: CN1CCN(CCOc2ccc3cc2OC/C=C/CN2C(=O)N(c4c(CF)csc4Cl)Cc4cnc(nc42)N3)CC1 ; CN(C)CCOc1ccc2cc1COC/C=C\CN1C(=O)N(c3c(CF)csc3Cl)Cc3cnc(nc31)N2 ; CN(C)CCOc1ccc2cc1OC/C=C\CN1C(=O)N(c3c(CF)csc3Cl)Cc3cnc(nc31)N2]

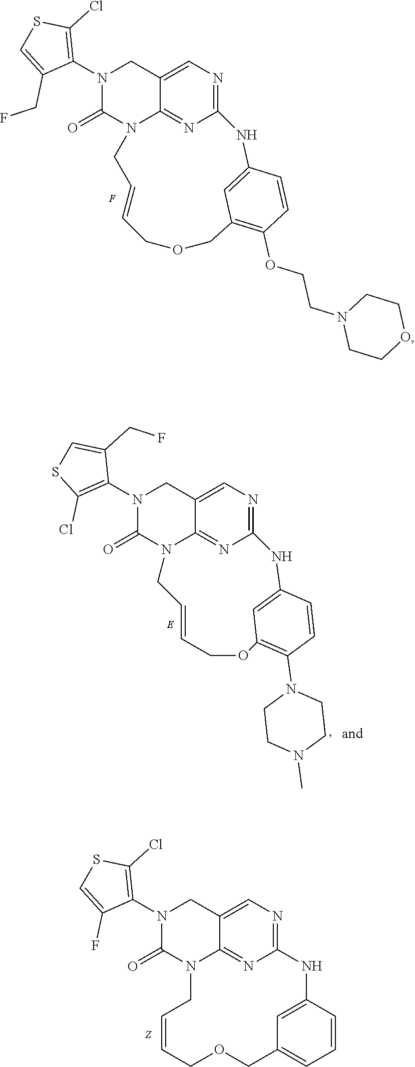

and solvates, salts, N-oxides, complexes, polymorphs, crystalline forms, tautomers, conformers, isotopically labeled forms, prodrugs, and combinations thereof.
ITEM 68. The compound of any one of items 1 to 67, wherein the compound is in substantially pure form, in particular in greater than about 90%, 95%, 98% or 99% pure form.
ITEM 69. A pharmaceutical composition comprising the compound of any one of items 1 to 68, and optionally further comprising a pharmaceutically acceptable excipient.
ITEM 70. The pharmaceutical composition of item 69 formulated for oral administration.
ITEM 71. The pharmaceutical composition of item 69 or 70 in unit dose form.
ITEM 72. The compound of any one of items 1 to 68, or the pharmaceutical composition of any one of items 69 to 71, for use in therapy.
ITEM 73. A method for the treatment of a disease, disorder or condition in a subject, comprising administering to the subject a compound of any one of items 1 to 68, or a pharmaceutical composition of any one of items 69 to 71, optionally wherein the disease, disorder or condition is associated with a kinase, such as one or more disclosed herein.
ITEM 74. A method for the treatment of a proliferative disorder in a subject, comprising administering to the subject a compound of any one of items 1 to 68, or a pharmaceutical composition of any one of items 69 to 71.
ITEM 75. A compound for use, or a pharmaceutical composition for use, in a treatment of a proliferative disorder in a subject, the treatment comprising administering the compound or the pharmaceutical composition to the subject, wherein, the compound is a compound of any one of items 1 to 68, and the pharmaceutical composition is a pharmaceutical composition of any one of items 69 to 71, optionally wherein the disease, disorder or condition is associated with a kinase, such as one or more disclosed herein.
ITEM 76. The compound for use, or the pharmaceutical composition for use, of item 75, wherein the proliferative disorder is a cancer or tumour.
ITEM 77. The compound for use, or the pharmaceutical composition for use, of item 76, wherein the cancer is a solid tumour.
ITEM 78. The compound for use, or the pharmaceutical composition for use, of any one of items 72 and 75 to 77, wherein treatment further comprises administration of an immune checkpoint inhibitor to the subject.
ITEM 79. The compound for use, or the pharmaceutical composition for use, of any one of items 72 and 75 to 78, wherein: (i) the proliferative disorder has progressed on standard therapy in the subject; or (ii) the subject is unable to receive standard therapy.
ITEM 80. The compound for use, or the pharmaceutical composition for use, of any one of items 72 and 75 to 79, wherein the treatment involves inhibiting SIK3 in the subject.
ITEM 81. The compound for use, or the pharmaceutical composition for use, of any one of items 72 and 75 to 80, wherein the treatment involves sensitising cells involved with the proliferative disorder in the subject to a cell-mediated immune response.
ITEM 82. The compound for use, or the pharmaceutical composition for use, of any one of items 72 and 75 to 81, wherein the compound or pharmaceutical composition is administered to the subject to sensitise cells involved with the proliferative disorder to killing induced by TNF.
ITEM 83. The compound for use, or the pharmaceutical composition for use, of any one of items 72 and 75 to 82, the treatment comprising exposing cells involved with the proliferative disorder in the subject to: (i) TNF, a TNF variant, and/or an agonist of TNFR1- or TNFR2-signalling; and (ii) the compound or pharmaceutical composition.
ITEM 84. The compound for use, or the pharmaceutical composition for use, of item 83, wherein the amount of TNF exposed to cells involved with the proliferative disorder in the subject is increased.
ITEM 85. The compound for use, or the pharmaceutical composition for use, of item 83 or 84, wherein: (i) TNF, a TNF variant or an agonist of TNFR1- or TNFR2-signalling is administered to the subject; (ii) an agent that is capable of inducing or induces the exposure of the cells involved with the proliferative disorder to TNF, a TNF variant or an agonist of TNFR1- or TNFR2-signalling, is administered to the subject; or (iii) the exposure of the cells involved with the proliferative disorder to TNF is induced by a pharmaceutical, therapeutic or other procedure that increases the amount of TNF in the plasma of the subject and/or in the environment of such cells.
ITEM 86. The compound for use, or the pharmaceutical composition for use, of any one of items 83 to 85, wherein the exposure of the cells involved with the proliferative disorder to TNF is induced by a pharmaceutical, therapeutic or other procedure that increases the amount of TNF in the plasma of the subject and/or in the environment of such cells.
ITEM 87. A compound for use, or a pharmaceutical composition for use, in a treatment of a proliferative disorder in a subject, the treatment comprising administering the compound or the pharmaceutical composition to the subject, wherein the compound is selected from any one of items 1 to 68, and the pharmaceutical composition comprises such a compound and, optionally, a pharmaceutically acceptable excipient.
ITEM 88. The compound or a pharmaceutical composition for use of item 87, wherein the compound is selected from the group consisting of:

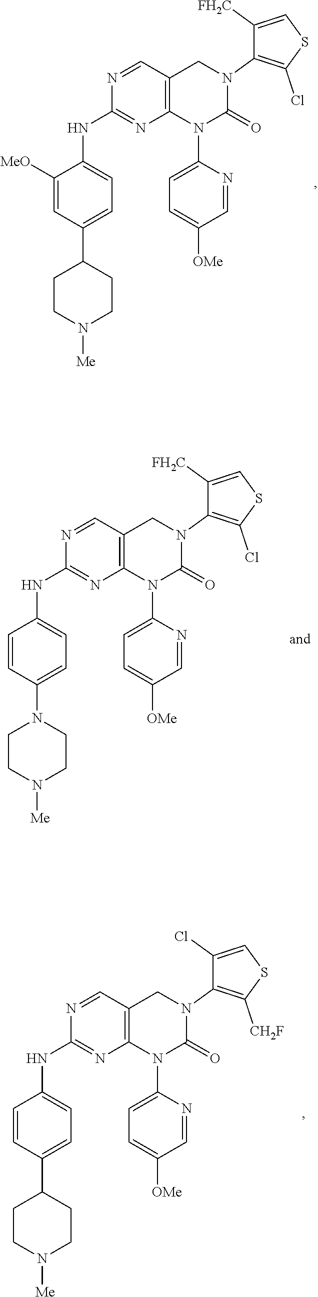

and solvates, salts, N-oxides, complexes, polymorphs, crystalline forms, tautomers, conformers, isotopically labeled forms, prodrugs, and combinations thereof.
ITEM 89. A method for the treatment of a proliferative disorder in a subject, comprising administering to the subject a compound or pharmaceutical composition as defined in item 87, wherein the proliferative disorder is as defined in item 87.
ITEM 90. The compound for use, or pharmaceutical composition for use, of item 87 or 88 or the method of item 89, wherein the proliferative disorder is characterised by, or cells involved with the proliferative disorder characterised by: (i) the presence of a human chromosomal translocation at 11q23; (ii) the presence of a rearrangement of the KMT2A gene; and/or (iii) the presence of an KMT2A fusion oncoprotein, preferably wherein:
(a) the human chromosome translocation is one selected from the group consisting of: t(4,11), t(9,11), t(11,19), t(10,11) and t(6,11); and/or
(b) the rearrangement of the KMT2A gene comprises, or the KMT2A fusion oncoprotein is expressed from a rearrangement that comprises, a fusion of the KMT2A gene with a translocation partner gene selected from the group consisting of: AF4, AF9, ENL, AF10, ELL and AF6.
ITEM 91. The compound for use, or pharmaceutical composition for use, of any one of items 87, 88 and 90 or the method of item 89 or 90, wherein the proliferative disorder is a cancer or a tumour, preferably a haematopoietic malignancy and/or a lymphoid malignancy.
ITEM 92. The compound for use, or pharmaceutical composition for use, of any one of items 87, 88, 90 and 91, or the method of any one of items 89 to 91, wherein the proliferative disorder is: (i) a myeloma, preferably multiple myeloma; or (ii) a leukaemia, preferably an acute myeloid leukaemia (AML) or an acute lymphoblastic leukaemia (ALL), more preferably T cell acute lymphoblastic leukaemia (T-ALL), an MLL-AML or an MLL-ALL.
ITEM 93. The compound for use, or pharmaceutical composition for use, of any one of items 87, 88 and 90 to 92, or the method of any one of items 89 to 92, wherein the subject is a human paediatric patient and/or is a subject carrying a KMT2A rearrangement (KMT2A-r); preferably wherein the subject is a patient suffering from a KMT2A-r leukaemia.
ITEM 94. A method for determining that a subject suffering from a proliferative disorder is suitable for treatment with a compound or pharmaceutical composition as defined in item 87 or 88, the method comprising, determining in a biological sample that has been obtained from said subject, and preferable that comprises cells involved with the proliferative disorder:
(X) the presence of MEF2C protein, such as of phosphorylated MEF2C protein and/or of MEF2C protein as an active transcription factor; preferably wherein the proliferative disorder is further characterised by the presence of phosphorylated HDAC4 protein, such as of HDAC4 protein phosphorylated by SIK3; and/or
(Y) (i) the presence of a human chromosomal translocation at 11q23; (ii) the presence of a rearrangement of the KMT2A gene; (iii) the presence of an KMT2A fusion oncoprotein; and/or (iv) the presence of a mutation in the KRAS gene and/or in the RUNX1 gene,
wherein, the presence of said protein, translocation, rearrangement, oncoprotein and or mutation in the biological sample indicates that the subject is suitable for treatment with the compound or pharmaceutical composition.
ITEM 95. The method of item 94, comprising determining in a biological sample that has been obtained from said subject: (i) the presence of a human chromosomal translocation at 11q23; (ii) the presence of a rearrangement of the KMT2A gene; and/or (iii) the presence of an KMT2A fusion oncoprotein, preferably wherein:
(a) the human chromosome translocation is one selected from the group consisting of: t(4,11), t(9,11), t(11,19), t(10,11), and t(6,11); and/or
(b) the rearrangement of the KMT2A gene comprises, or the KMT2A fusion oncoprotein is expressed from a rearrangement that comprises, a fusion of the KMT2A gene with a translocation partner gene selected from the group consisting of: AF4, AF9, ENL, AF10, ELL and AF6.
ITEM 96. The method of item 94 or 95, wherein the proliferative disorder is a cancer or a tumour, preferably a haematopoietic malignancy and/or a lymphoid malignancy.
ITEM 97. The method of any one of items 94 to 96, wherein the proliferative disorder is: (i) a myeloma, preferably multiple myeloma; or (ii) a leukaemia, preferably an acute myeloid leukaemia (AML) or an acute lymphoblastic leukaemia (ALL), more preferably T cell acute lymphoblastic leukaemia (T-ALL), an MLL-AML or an MLL-ALL.
ITEM 98. The method of any one of items 94 to 97, wherein the subject is a human paediatric patient and/or is a subject carrying a KMT2A rearrangement (KMT2A-r); preferably wherein such subject is a patient suffering from a KMT2A-r leukaemia.
ITEM 99. The method of any one of items 94 to 98, further comprising a step of administering a compound or pharmaceutical composition as defined in item 87 or 88 to a subject where the presence of, or an amount of, said protein, translocation, oncoprotein and or mutation is determined in a biological sample that had been obtained from said subject.
ITEM 100. A method of preparing a compound of item 68, comprising the steps:
providing a compound of any one of items 1 to 67 in admixture with one or more impurities; and
removing at least a fraction of the impurities from the admixture.
   ITEM 101. A method of manufacturing a pharmaceutical composition comprising the step of formulating a compound of any one of items 1 to 68 together with a pharmaceutically acceptable excipient.
   ITEM 102. A method of preparing a pharmaceutical package, comprising the steps:
inserting into packaging a pharmaceutical composition of any one of items 69 to 71 (preferably in finished pharmaceutical form), thereby forming a package containing the pharmaceutical composition; and optionally,
inserting into the package a leaflet describing prescribing information for the pharmaceutical composition.
   ITEM 103. A pharmaceutical package containing a pharmaceutical composition of any one of items 69 to 71; preferably, wherein the pharmaceutical composition is in finished pharmaceutical form.

### Examples

A selection of compounds within the scope of, or for use within the methods of, the present invention—and/or that represent examples of various exemplary or preferred R¹ substituents (such as R^{1a} substituents and/or Q substituents), R² substituents, R³ substituents, R⁴ substituents, R⁵ moieties (such as L moieties, R⁶ substituents and/or R⁷ substituents), E moieties and/or L′ moieties, each individually or in any combination are useful for synthesising further compounds of the invention—is listed in Table A and/or Table B. A selection of the compounds shown in Table A and/or Table B are synthesised and tested as described herein.

The examples show:

### Example 1.1: Synthesis of Prior Art Compound PY1 and Various Compounds of Formula (I)

### General Methods and Materials:

The urea formation was performed using a Discovery Microwave Synthesizer. All reactions were monitored by TLC with 0.25 mm E. Merck precoated silica gel plates (60 F254) and Waters liquid chromatography-mass spectroscopy (LCMS). LC-MS spectra were recorded on a Waters Acquity I class UPLC system using the following system [solvent A: acetonitrile, solvent B: 0.1% formic in water or solvent A: acetonitrile, solvent B: 0.1% ammonia in water. Formic acid and ammonia was used as HPLC grade. All the separations were performed at ambient temperatures.

Reverse phase HPLC was performed on a Waters HPLC system using following system [solvent A: acetonitrile, solvent B: 0.2% NH₃ in water]. Ammonia was used as HPLC grade. All the separations were performed at ambient temperatures. For analytical RP-HPLC analysis [Interchim: Acquity BEH C18 (2.1×100 mm, 1.7 um)], the flow rate was 0.4 ml·min⁻¹; injection volume: 10 μL, detection wavelengths: 220 nm and 254 nm. The following gradient was used: 0.01 min 90% B, over 8 min to 10% B, 4 min 10% B.

Purification of reaction products was carried out by column chromatography using commercially available silica or flash chromatography using Combiflash Rf with Teledyne IscoRediSepRfHigh Performance Gold or SilicycleSiliaSep High Performance columns (40, 80, or 120 g). The purity of all final compounds was over 95% and was analyzed with Waters LCMS system.

¹H NMR spectra were recorded on Bruker 300 MHz spectrometers and are reported in ppm with the solvent resonance employed as the internal standard [CDCl₃ at 7.26 ppm, DMSO-d6 at 2.50 ppm]. Peaks are reported as (s=singlet, d=doublet, t=triplet, q=quartet, m=multiplet or unresolved, bs=broad signal, coupling constant(s) in Hz, integration).

Abbreviations: Boc₂O: di-tert-butyl decarbonate; 1-BuOH: 1-butanol; cHex: cyclohexane; CV: column value; d: doublet (NMR); d: days; dd: doublet of doublets; DCM=CH₂Cl₂: dichloromethane; DIPEA: N,N-diethylisopropyl amine; DMF: N,N-dimethylformamide; DMSO: dimethyl sulfoxide; EDC HCl: 1-Ethyl-3-(3-dimethylaminopropyl)carbodiimid; equiv.: equivalents; Et₂O: diethyl ether; EtOAc: ethyl acetate; g: gram; h: hours; H: proton; HCl: hydrochloric acid; H₂O: water; HOBT: 1-hydroxybenzotriazol; Hz: Hertz; IPA: iso-propanol; 1: scalar ¹H-¹H coupling constant; K₂CO₃: potassium carbonate; KOH: potassium hydroxide; LC-MS: liquid chromatography-mass spectrometry; m: multiplet; M: molar; mAU: milli absorption units; Me: methyl; MeCN: acetonitrile; MeOH:methanol; mg: milli gram; MHz: mega Hertz: min: minutes; pw: microwave; N₂: nitrogen; NaH: sodium hydride; NaHCO₃: sodium bicarbonate; NaOH: sodium hydroxide; Na₂SO₄: sodium sulfate; NBS: N-bromo succinimid; NCS: N-chloro succinimid; NMR: nuclear magnetic resonance; PdCl₂(dppf): [1,1′-Bis(diphenylphosphino)ferrocene]dichloropalladium (II); Pd₂dba₃: Tris(dibenzylideneacetone)dipalladium(0); quant.: quantitative; R_{f}: retention factor (TLC); rt: room temperature; s: singlet; SiO₂: silica; TCFH: tetramethylchloroformamidinium hexafluorophosphate; TFA: Trifluoro acetic acid; THF: Tetrahydrofuran; TLC: thin layer chromatography; XantPhos: 4,5-Bis(diphenylphosphino)-9,9-dimethylxanthene.

### General Synthesis Schemes:

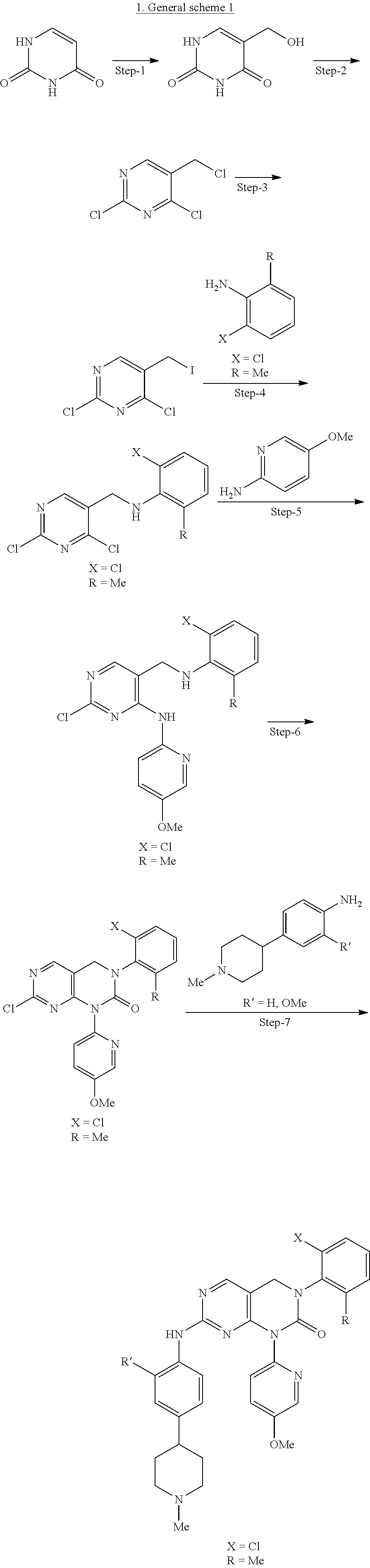

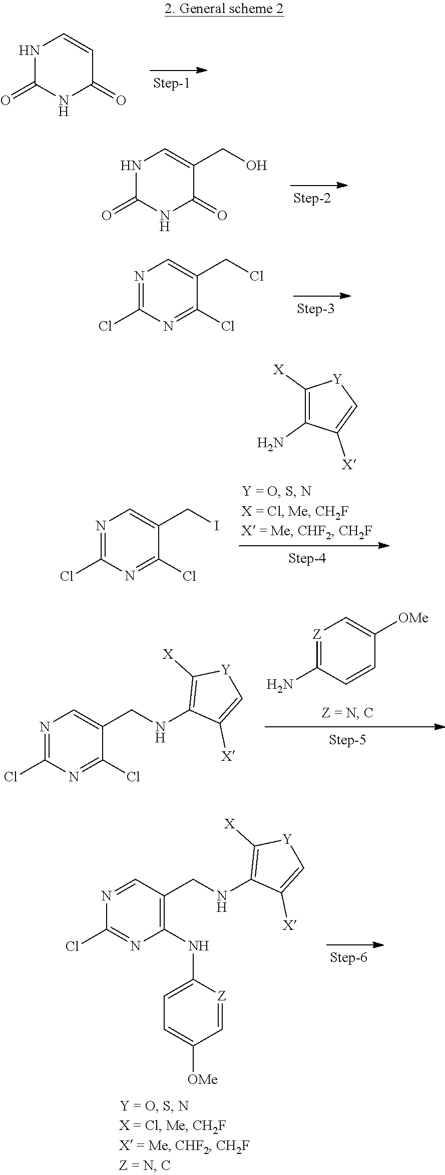

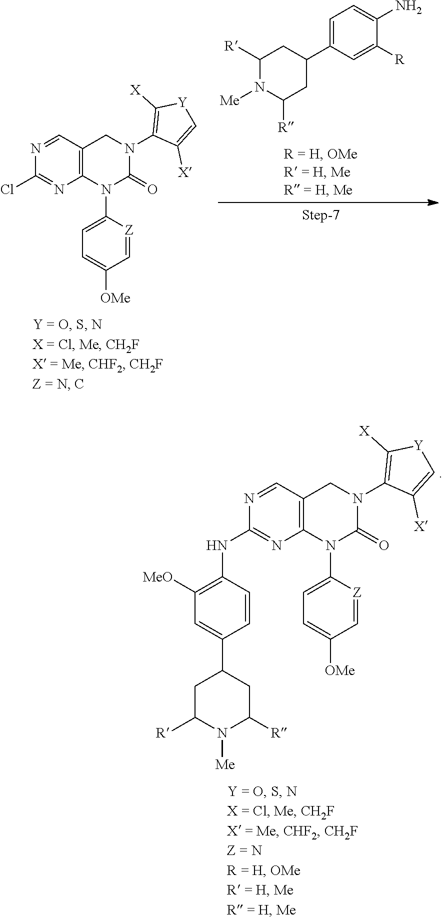

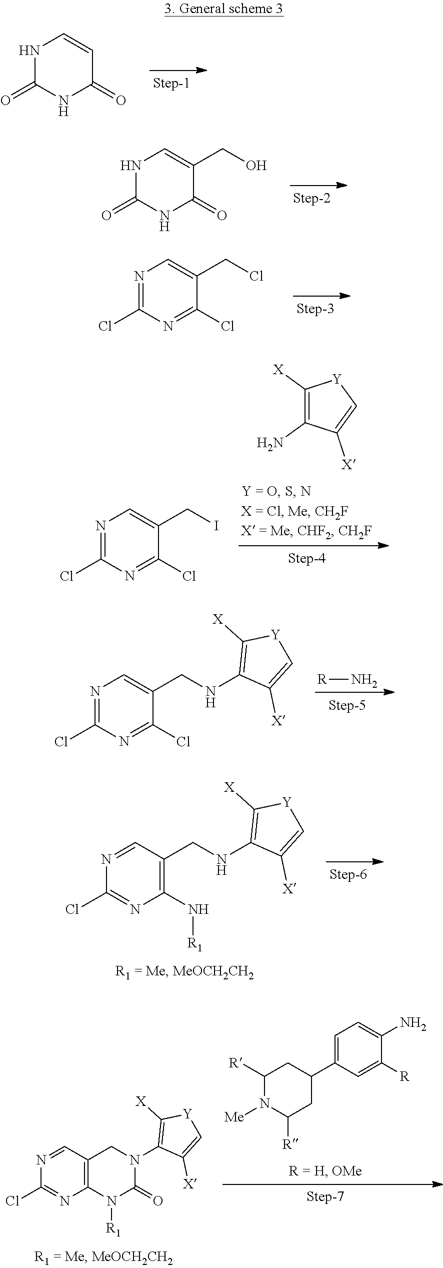

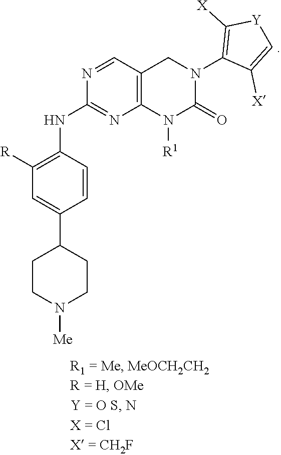

### Compound PY1

### Synthesis of 3-(2-chloro-6-methylphenyl)-7-(2-methoxy-4-(1-methylpiperidin-4-yl) phenylamino)-1-(5-methoxypyridin-2-yl)-3,4-dihydropyrimido[4,5-d]pyrimidin-2(1H)-one (PY1)

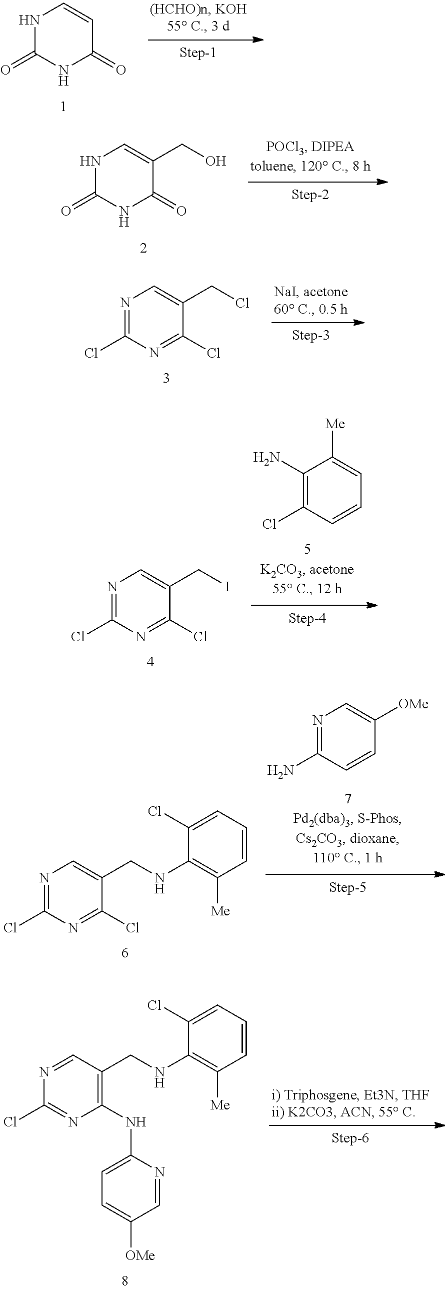

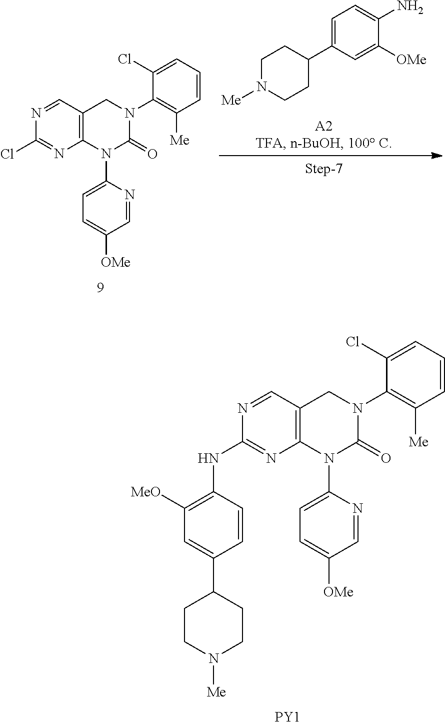

### Step-1: 5-(hydroxymethyl) pyrimidine-2,4(1H,3H)-dione (2)

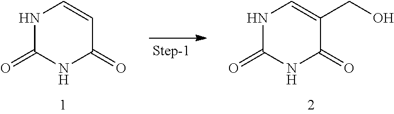 [SMILES: O=c1cc[nH]c(=O)[nH]1 ; O=c1[nH]cc(CO)c(=O)[nH]1]

To a solution of the KOH (28.3 g, 505.62 mmol 0.56 eq) in H₂O (780 mL), paraformaldehyde (33.2 g, 1106.2 mmol) and uracil (1) (100 g, 892.85 mmol, 1.0 eq) was added. The reaction was heated to 55° C. for 16 h and progress of the reaction was monitored by TLC. After complete consumption of starting material, the reaction mixture was concentrated under reduced pressure to get the crude solid compound. The solid was filtered, washed with acetone and dried under vacuo to get 5-(hydroxymethyl)pyrimidine-2,4(1H,3H)-dione (2) (110.4 g 87.3%) as a white solid. ¹H NMR (300 MHz, DMSO-d6): δ 7.31 (s, 1H), 4.06 (s, 2H).

### Step-2: 2,4-dichloro-5-(chloromethyl)pyrimidine (3)

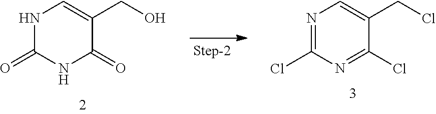 [SMILES: O=c1[nH]cc(CO)c(=O)[nH]1 ; ClCc1cnc(Cl)nc1Cl]

To a solution of 5-(hydroxymethyl)pyrimidine-2,4(1H,3H)-dione (2) (110 g, 774.64 mmol, 1.0 eq) in toluene (330 mL) cooled to 0° C. and POCl₃ (546.6 g 3564.8 mmol 4.6 eq) added drop wise followed by DIPEA (279.1 g 2159.38 mmol 2.79 eq). The reaction mixture was heated to 120° C. for 16 h. The progress of the reaction was monitored by TLC. The TLC shows complete consumption of starting material. The reaction mixture was poured to mixture of ice water and ethyl acetate separated the organic layer and washed with brine solution dried over Na₂SO₄ then concentrated the solvent under reduced pressure to get the crude compound. The residue was purified by column chromatography on silica gel to afford 2,4-dichloro-5-(chloromethyl)pyrimidine (3) (59.1 g, 38.6%) as a off-white solid. ¹H NMR (300 MHz, DMSO-d6): δ 8.66 (s, 1H), 4.64 (s, 2H). LCMS: m/z=196.87 [M+H]⁺, 99.37% (2.99 min).

### Step-3: 2,4-dichloro-5-(iodomethyl)pyrimidine (4)

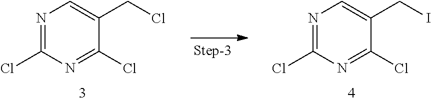 [SMILES: ClCc1cnc(Cl)nc1Cl ; Clc1ncc(CI)c(Cl)n1]

A solution of 2,4-dichloro-5-(chloromethyl)pyrimidine (3) (59.0 g, 301.02 mmol, 1.0 eq.), NaI (54.14 g, 361.22 mmol, 1.2 eq.) in Acetone (350 mL) was stirred at 60° C. for 1 h. The progress of the reaction was monitored by TLC. After complete consumption of starting material, the reaction mixture was filtered and the filtrate was concentrated to get crude compound. The residue was purified by column chromatography on silica gel to afford 2,4-dichloro-5-(iodomethyl)pyrimidine (4) (70.1 g, 81.39%) as a off-white solid. ¹H NMR (300 MHz, CDCl₃): δ 8.60 (s, H), 4.38 (s, 2H). LCMS: m/z=288.87 [M+H]⁺, 94.88% (3.22 min)

### Step-4: 2-chloro-N-((2,4-dichloropyrimidin-5-yl)methyl)-6-methylaniline (6)

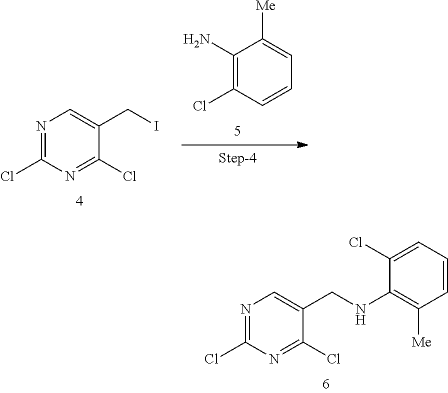 [SMILES: Cc1cccc(Cl)c1N ; Clc1ncc(CI)c(Cl)n1 ; Cc1cccc(Cl)c1NCc1cnc(Cl)nc1Cl]

To a solution of 2,4-dichloro-5-(iodomethyl)pyrimidine (4) (5.0 g, 17.36 mmol, 1.0 eq.) and 2-chloro-6-methylaniline (5) (3.2 g, 21.98 mmol 1.3 eq.) in Acetone (30 mL) was added K₂CO₃(7.2 g, 52.09 mmol, 3.0 eq.) and stirred at 55° C. for 12 h. The progress of the reaction was monitored by TLC. The TLC shows complete consumption of starting material. The reaction mixture was filtered and the filtrate was concentrated to get crude compound. The residue was purified by column chromatography on silica gel to afford 2-chloro-N-((2,4-dichloropyrimidin-5-yl)methyl)-6-methylaniline (6) (4.1 g, 76.92%) as a gummy liquid. ¹H NMR (400 MHz, DMSO-d6): δ 8.84 (s, 1H), 7.19-7.17 (d, J=8.8 Hz, 1H), 7.11-7.09 (d, J=8.4 Hz, 1H), 6.88-6.84 (t, J=15.2 Hz, 1H) 4.991 (s, 1H), 4.33-4.32 (d, J=6.4 Hz, 2H), 2.27 (s, 3H). LCMS: m/z=302.00 [M+H]⁺, 89.28% (3.18 min).

### Step-5: 2-chloro-5-((2-chloro-6-methylphenylamino) methyl)-N-(5-methoxypyridin-2-yl) pyrimidin-4-amine (8)

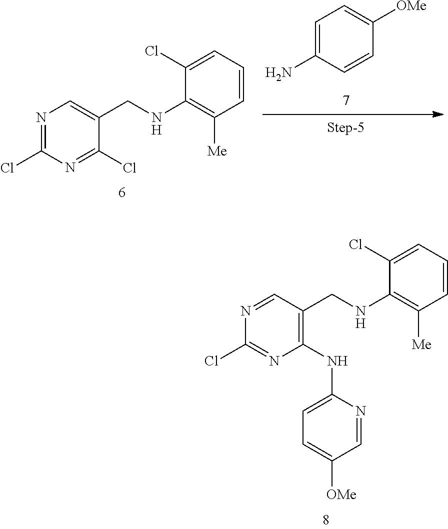

A mixture of 2-chloro-N-((2,4-dichloropyrimidin-5-yl)methyl)-6-methylaniline (6) (4.0 g, 13.28 mmol, 1.0 eq.), 5-methoxypyridin-2-amine (7) (1.97 g, 15.87 mmol 1.2 eq.), Cs₂CO₃ (3.64 g, 11.16 mmol, 1.5 eq.) in dioxane (40 mL) was degassed for 10 min. To a reaction mixture was added Pd₂ (dba)₃ (1.2 g, 1.311 mmol, 0.1 eq.) and S-Phos (1.1 g, 2.679 mmol 0.2 eq.) and heated at 110° C. for 4 h. The progress of the reaction was monitored by TLC. The TLC shows complete consumption of starting material. Reaction mixture was filtered with a pad of celite and concentrated under reduced pressure. The residue was purified by column chromatography on silica gel to afford 2-chloro-5-((2-chloro-6-methylphenylamino) methyl)-N-(5-methoxypyridin-2-yl) pyrimidin-4-amine (8) (0.9 g, 15.5%) as a off-white solid. ¹H NMR (400 MHz, DMSO-d6): δ 10.01 (s, 1H), 8.09 (d, J=2.0 Hz, 1H), 8.01 (d, J=9.2 Hz, 1H), 7.45 (dd, J=9.2, 3.2 Hz, 1H), 7.21 (d, 3=7.2 Hz, 1H), 7.12 (d, J=7.6 Hz, 1H), 6.91 (t, J=8.0 Hz, 1H), 4.92 (t, 3=7.6 Hz, 1H), 4.24 (d, J=7.2 Hz, 2H), 3.83 (s, 3H), 2.31 (s, 3H). LCMS: m/z=390.00 [M+H]⁺, 86.24% (6.53 min).

### Step-6: 7-chloro-3-(2-chloro-6-methylphenyl)-1-(5-methoxypyridin-2-yl)-3,4-dihydropyrimido[4,5-d]pyrimidin-2(1H)-one (9)

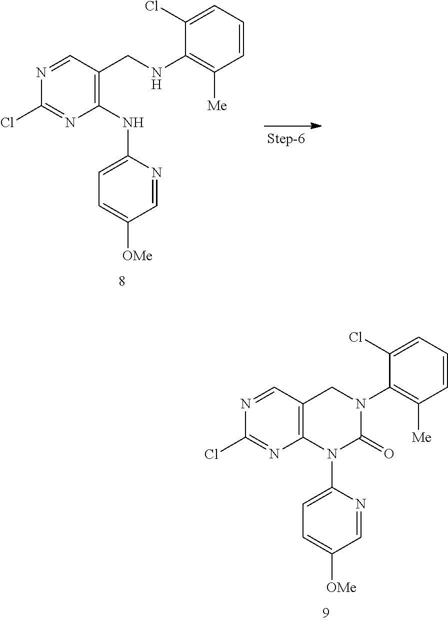 [SMILES: COc1ccc(Nc2nc(Cl)ncc2CNc2c(C)cccc2Cl)nc1 ; COc1ccc(N2C(=O)N(c3c(C)cccc3Cl)Cc3cnc(Cl)nc32)nc1]

To a solution of 2-chloro-5-((2-chloro-6-methylphenylamino)methyl)-N-(5-methoxypyridin-2-yl)pyrimidin-4-amine (8) (0.9 g, 2.307 mmol 1.0 eq.) in Dry THF (8.0 mL) Triphosgene (750 mg, 2.527 mmol 1.1 eq.) in THF (2 mL) were added followed by Et₃N (280 mg, 2.767 mmol 1.2 eq.) added, then stirred at Room temperature for 2 h, To the reaction mixture K₂CO₃ (3.18 g 23.01 mmol 10.0 eq.) and ACN were added, then stirred at 55° C. for 16 h. The progress of the reaction was monitored by TLC. The TLC shows complete consumption of starting material. The reaction mixture was filtered and the filtrate was concentrated to get crude compound. The residue was purified by column chromatography on silica gel to afford 7-chloro-3-(2-chloro-6-methylphenyl)-1-(5-methoxypyridin-2-yl)-3,4-dihydropyrimido[4,5-d]pyrimidin-2(1H)-one (9) (0.5 g, 52.63%) as a off-white solid. ¹H NMR (300 MHz, DMSO-d6): δ 8.46 (s, 1H), 8.27 (d, J=4.0 Hz, 1H), 8.01 (d, J=9.2 Hz, 1H), 7.59-7.55 (m, 1H), 7.48-7.41 (m, 1H), 7.36-7.30 (m, 1H), 4.83 (s, 2H), 3.90 (s, 3H), 2.31 (s, 3H). LCMS: m/z=416.09 [M+H]⁺, 97.52% (6.58 min).

### Step-7: 3-(2-chloro-6-methylphenyl)-7-(2-methoxy-4-(1-methylpiperidin-4-yl) phenylamino)-1-(5-methoxypyridin-2-yl)-3,4-dihydropyrimido[4,5-d]pyrimidin-2(1H)-one (PY1)

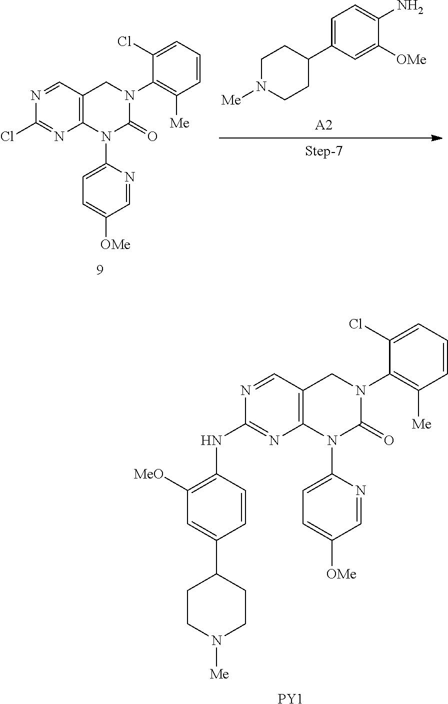

To a solution of 7-chloro-3-(2-chloro-6-methylphenyl)-1-(5-methoxypyridin-2-yl)-3,4-dihydropyrimido[4,5-d]pyrimidin-2(1H)-one (9) (0.5 g, 1.201 mmol 1.0 eq.) and 2-methoxy-4-(1-methylpiperidin-4-yl)aniline (A2) (0.32 g 1.424 mmol 1.2 eq) in n-BuOH (2 mL) was added Trifluroacetic acid (0.18 g, 1.538 mmol 1.3 eq.) and stirred at 100° C. for 6 h. The progress of the reaction was monitored by TLC. The TLC shows complete consumption of starting material The reaction mixture was concentrated and was purified with Combiflash Rf with Teledyne IscoRediSepRfHigh Performance Gold or SilicycleSiliaSep High Performance columns (40, 80, or 120 g) to afford 3-(2-chloro-6-methylphenyl)-7-(2-methoxy-4-(1-methylpiperidin-4-yl)phenylamino)-1-(5-methoxypyridin-2-yl)-3,4-dihydropyrimido[4,5-d]pyrimidin-2(1H)-one (PY1) (100 mg, 14%) as a white solid. Analytical data for this synthesised compound PY1 is shown in Table 1.1.

### Preparation of 5-methoxypyridin-2-amine (7)

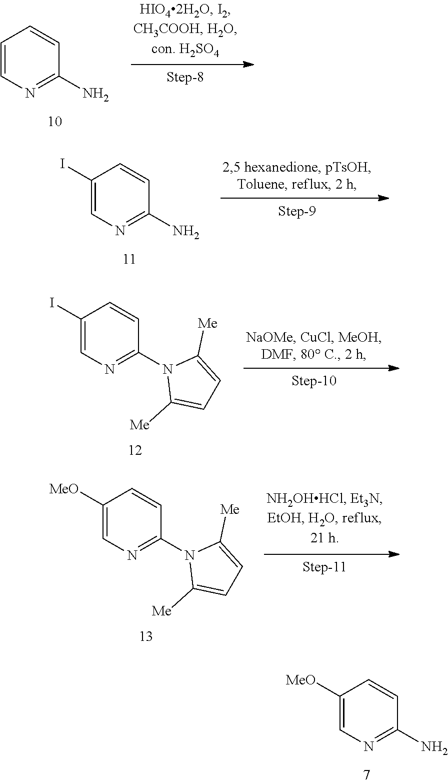 [SMILES: Nc1ccccn1 ; Nc1ccc(I)cn1 ; Cc1ccc(C)n1-c1ccc(I)cn1 ; COc1ccc(-n2c(C)ccc2C)nc1 ; COc1ccc(N)nc1]

### Step-8: 2-amino-5-iodopyridine (11)

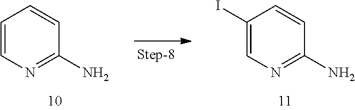 [SMILES: Nc1ccccn1 ; Nc1ccc(I)cn1]

A mixture of 2-aminopyridine (10) (100.0 g, 1062.6 mmol, 1.0 equiv.), periodic acid dihydrate (10.7 g, 160.25 mmol, 0.15 equiv.) and iodine (32.4 g, 446.6 mmol, 0.42 equiv.) was heated in a mixed solution of acetic acid (2.6 L), water (120 ml) and sulfuric acid (20.6 ml) at 80° C. for 4 h. The reaction mixture was poured into 10% aqueous Na₂S₂O₃ solution to quench any remaining iodine and extracted with ether. The extract was washed with 10% aqueous NaOH solution, dried (MgSO₄) and concentrated in vacuo. The residue was purified by column chromatography to give 2-amino-5-iodopyridine (11) (163.1 g, =70%). as a yellow solid. LCMS: m/z=220.89 [M+H]⁺, 99.35% (1.66 min).

### Step-9: 2-(2,5-dimethyl-1H-pyrrol-1-yl)-5-iodopyridine (12)

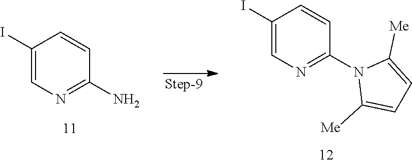 [SMILES: Nc1ccc(I)cn1 ; Cc1ccc(C)n1-c1ccc(I)cn1]

2-Amino-5-iodopyridine (11) (163.0 g, 741.3 mmol, 1.0 equiv), 2,5-hexanedione (100.4 g 888.6 mmol, 1.2 equiv.) and p-toluenesulfonic acid (12.7 g, 73.68 mmol, 0.1 equiv.) were dissolved in toluene (670 ml) and heated in a Dean-Stark apparatus for 5 h. After cooling to room temperature, the dark brown reaction mixture was washed with a saturated aqueous NaHCO₃ solution, water and brine. The organic phase was dried with MgSO₄ and concentrated in vacuo. The resulting dark residue was dried under high vacuum and used in the next step without further purification (176.1 g=80%). LCMS: m/z=299.01 [M+H]⁺, 98.01% (3.91 min).

### Step-10: 2-(2,5-dimethyl-1H-pyrrol-1-yl)-5-methoxypyridine (13)

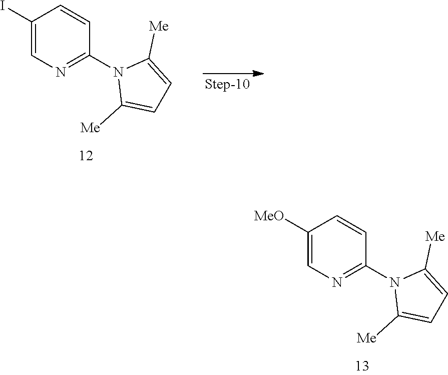 [SMILES: Cc1ccc(C)n1-c1ccc(I)cn1 ; COc1ccc(-n2c(C)ccc2C)nc1]

A mixture of 2-(2,5-dimethyl-1H-pyrrol-1-yl)-5-iodopyridine (12) (176.0 g, 590.6 mmol, 1.0 equiv.) in DMF (385 ml), CuI (16.8 g, 89.20 mmol, 0.15 equiv.) and sodium methoxide (95.3 g, 1765.2 mmol, 3.0 equiv.) were added. The reaction mixture was heated to 80° C. for 3 h. After the mixture had been allowed to room temperature, the solids were filtered off over Celite and the filtrate was extracted several times with dichloromethane. The combined organic phase was concentrated in vacuo to get the crude product. The residue was purified by column chromatography on silica gel to afford 2-(2,5-dimethyl-1H-pyrrol-1-yl)-5-methoxy-pyridine (13) (83.0 g, =70%) as a white solid. LCMS: m/z=203.03 [M+H]⁺, 92.64% (3.20 min).

### Step-11: 5-methoxypyridin-2-amine (7)

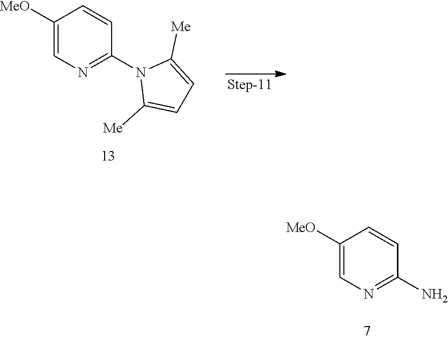 [SMILES: COc1ccc(-n2c(C)ccc2C)nc1 ; COc1ccc(N)nc1]

A mixture of 2-(2,5-dimethyl-1H-pyrrol-1-yl)-5-methoxypyridine (13) (83.0 g, 410.2 mmol, 1.0 equiv.), hydroxylamine hydrochloride (110.9 g, 2667 mmol, 6.5 equiv.), triethylamine (83.0 g, 820.4 mmol, 2.0 equiv.), ethanol (640 ml) and water (320 ml) was refluxed for 20 h. The solution was cooled and quenched with 2 M HCl, washed with isopropyl ether and the pH was adjusted to 9-10 with 6 M NaOH. The resulting mixture was extracted several times with dichloromethane. The combined organic phases were dried with MgSO₄ and the solvent was removed in vacuo. The residue was purified by column chromatography on silica gel to afford to give 2-amino-5-methoxy-pyridine (7) (40.7 g, 80%) as a gummy dark brown liquid. ¹H NMR (300 MHz, DMSO-d6): δ 3.66 (3H, s), 5.42 (brs, 2H), 6.42 (d, J=9.0 Hz, 1H), 7.09 (dd, J=11.6, 3.0 Hz, 1H), 7.64-7.63 (d, J=3.3 Hz, 1H). LCMS: m/z=124.99 [M+H]⁺, 98.56% (0.82 min).

### General Scheme for Synthesis of Intermediate—A1 & A2

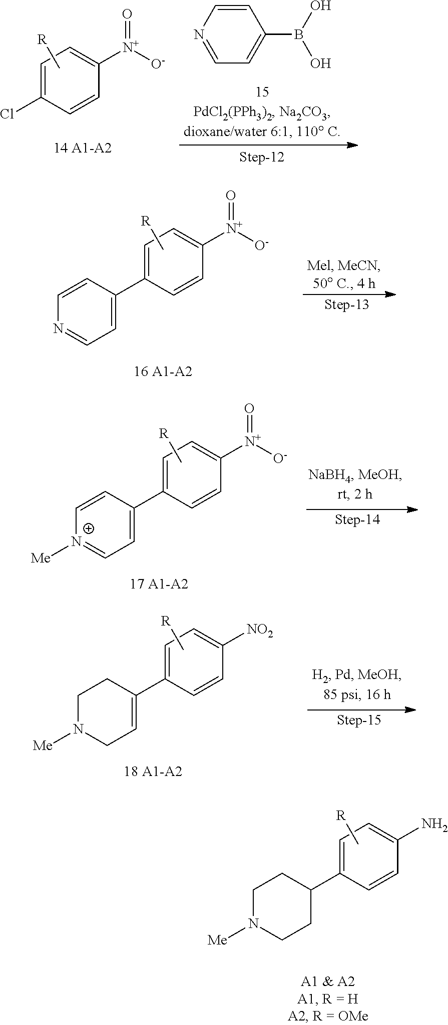

### Synthesis of Intermediate A1 & A2

### Step-12(a): 4-(4-nitrophenyl) Pyridine (16 A1)

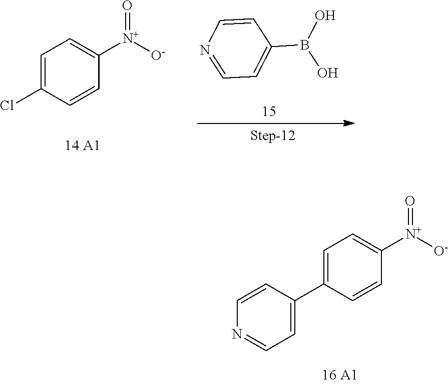 [SMILES: O=[N+]([O-])c1ccc(Cl)cc1 ; OB(O)c1ccncc1 ; O=[N+]([O-])c1ccc(-c2ccncc2)cc1]

To a mixture of pyridin-4-ylboronic acid (15) (2.46 g, 20.02 mmol, 1.0 equiv.), 1-bromo-4-nitrobenzene (14 A1) (4.42 g, 22 mmol, 1.0 equiv.), in dioxane: H2O (3:1.40 mL) Na₂CO₃(8.28 g, 60 mmol, 3.0 equiv) and Pd(dppf)Cl₂ (776 mg, 0.95 mmol, 0.03 equiv.) was added The mixture was stirred for 18 h under N2 at 100° C. After completion of the reaction, the mixture was cooled to room temperature, filtered, and the filtrate was concentrated to dryness. The residue was diluted with DCM, washed with water, dried over Na₂SO₄ and evaporated to dryness. The residue was purified by silica gel column chromatography to afford 4-(4-nitrophenyl) pyridine (16 A1) (1.64 g, 30%) as a white solid. LCMS: m/z=200.96 [M+H]⁺, 95.33% (2.71 min).

### Step-12(b): 4-(3-methoxy-4-nitrophenyl)pyridine (16 A2)

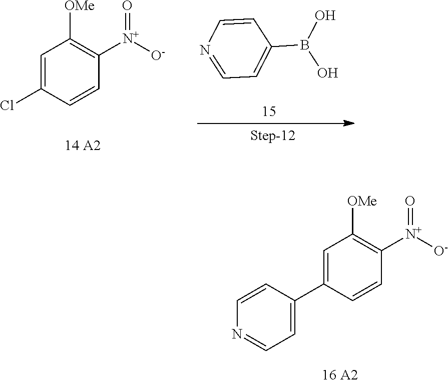 [SMILES: COc1cc(Cl)ccc1[N+](=O)[O-] ; OB(O)c1ccncc1 ; COc1cc(-c2ccncc2)ccc1[N+](=O)[O-]]

Synthesis of 16 A2 was same as above procedure. ¹H NMR (300 MHz, DMSO-d6): δ 8.72-8.70 (m, 2H), 8.01 (d, J=11.2 Hz, 1H), 7.84-7.82 (m, 2H), 7.68 (d, J=2.4 Hz, 1H), 4.0 (s, 3H).

### Step-13(a): 1-methyl-4-(4-nitrophenyl) Pyridinium (17 A1)

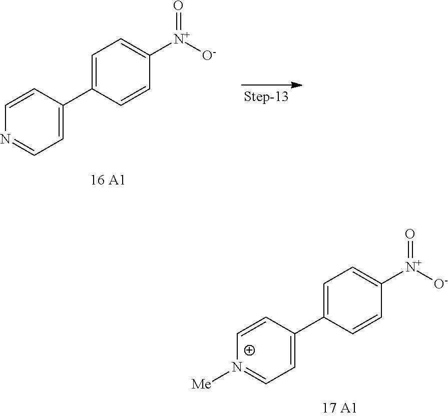 [SMILES: O=[N+]([O-])c1ccc(-c2ccncc2)cc1 ; C[n+]1ccc(-c2ccc([N+](=O)[O-])cc2)cc1]

A mixture of 4-(4-nitrophenyl)pyridine (16 A1) (1.6 g, 8 mmol, 1.0 equiv.) and MeI (3.9 g, 27.4 mmol, 3.5 equiv.) in 10 mL of acetonitrile was stirred for 4 h at 50° C. The solid that formed was collected by filtration, washed with cold acetonitrile, dried in vacuo to afford 1-methyl-4-(4-nitrophenyl)pyridinium (17 A1) (1.6 g, 90%) as a pale yellow solid. LCMS: m/z=215.01 [M], 98.72% (1.52 min).

### Step-13(b): 4-(3-methoxy-4-nitrophenyl)-1-methylpyridin-1-ium (17A2)

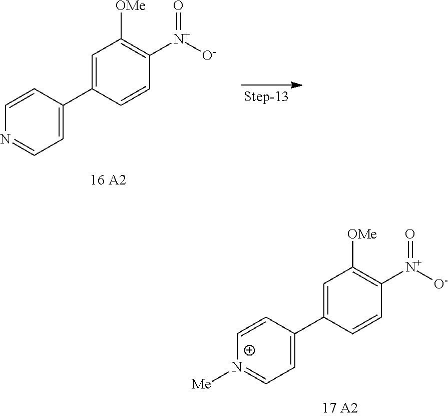 [SMILES: COc1cc(-c2ccncc2)ccc1[N+](=O)[O-] ; COc1cc(-c2cc[n+](C)cc2)ccc1[N+](=O)[O-]]

Synthesis of 17 A2 was same as above procedure. LCMS: m/z=245.04 [M+H]⁺, 81.33% (1.81 min).

### Step-14: 1-methyl-4-(4-nitrophenyl)-1,2,3,6-tetrahydropyridine (18 A1)

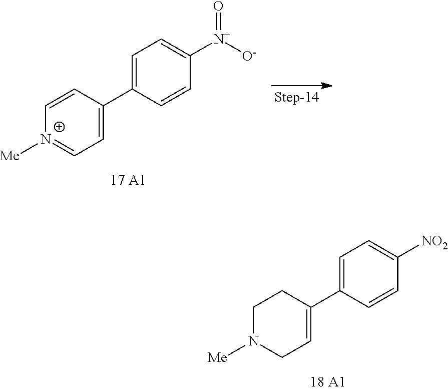 [SMILES: C[n+]1ccc(-c2ccc([N+](=O)[O-])cc2)cc1 ; CN1CC=C(c2ccc([N+](=O)[O-])cc2)CC1]

To a solution of 1-methyl-4-(4-nitrophenyl) pyridinium (17 A1) (1.56 g, 4.56 mmol, 1.0 equiv.) in 20 ml of MeOH was added NaBH₄ (0.52 g, 13.68 mmol, 10.0 equiv.) in portions at 0° C. The mixture was stirred for 2 h at room temperature. The mixture was treated with 40 mL of sat aq NaHCO₃. The solid that formed was collected by filtration and dissolved in 20 mL of 1.0 N HCl, washed with MTBE (2×20 mL). Then the aqueous phase was diluted with sat aq Na₂CO₃, extracted with DCM (3×30 mL), dried over Na₂SO₄ and evaporated to afford 1-methyl-4-(4-nitrophenyl)-1,2,3,6-tetrahydropyridine (18 A1) (850 mg, 85%) as a pale yellow solid. LCMS: m/z=219.06 [M+H]⁺, 99.61% (1.74 min).

### Step-15: Synthesis of 4-(1-methylpiperidin-4-yl)aniline (A1) & 2-methoxy-4-(1-methylpiperidin-4-yl)aniline (A2)

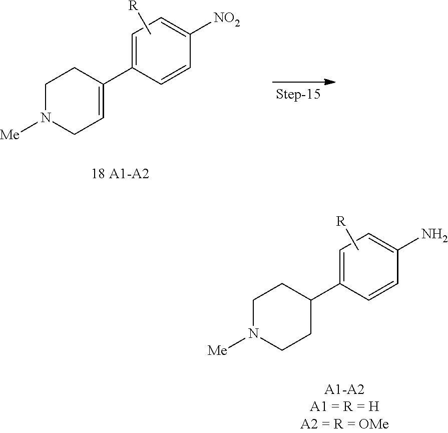

A1: A mixture of 1-methyl-4-(4-nitrophenyl)-1,2,3,6-tetrahydropyridine (18 A1) (850 mg, 3.88 mmol, 1.0 equiv.) and 0.4 g of Pd/C in 10 mL of MeOH was placed under 50 psi of H₂ gas for 16 h. The mixture was filtered and the filtrate was evaporated to afford 4-(1-methylpiperidin-4-yl) aniline (A1) (510 mg, 70%) as a white solid. 1H NMR (400 MHz, DMSO-d6): δ 6.87-6.85 (d, J=8.4 Hz, 2H), 6.49-6.46 (d, J=13.6 Hz, 2H), 4.80 (br s, 2H), 2.84-2.81 (m, 2H), 2.26-2.19 (m, 1H), 2.17 (s, 3H), 1.95-1.89 (m, 2H), 1.65-1.50 (m, 4H). LCMS: m/z=191.04 [M+H]⁺, 94.82% (0.70 min).

A2: Synthesis of A2 was same as above procedure. LCMS: m/z=221.02 [M+H]⁺, 93.69 (0.69 min).

### Compound AA1

### Synthesis of 3-(2-chloro-4-methylthiophen-3-yl)-7-(2-methoxy-4-(1-methylpiperidin-4-yl) phenylamino)-1-(5-methoxypyridin-2-yl)-3,4-dihydropyrimido[4,5-d]pyrimidin-2(1H)-one (AA1)

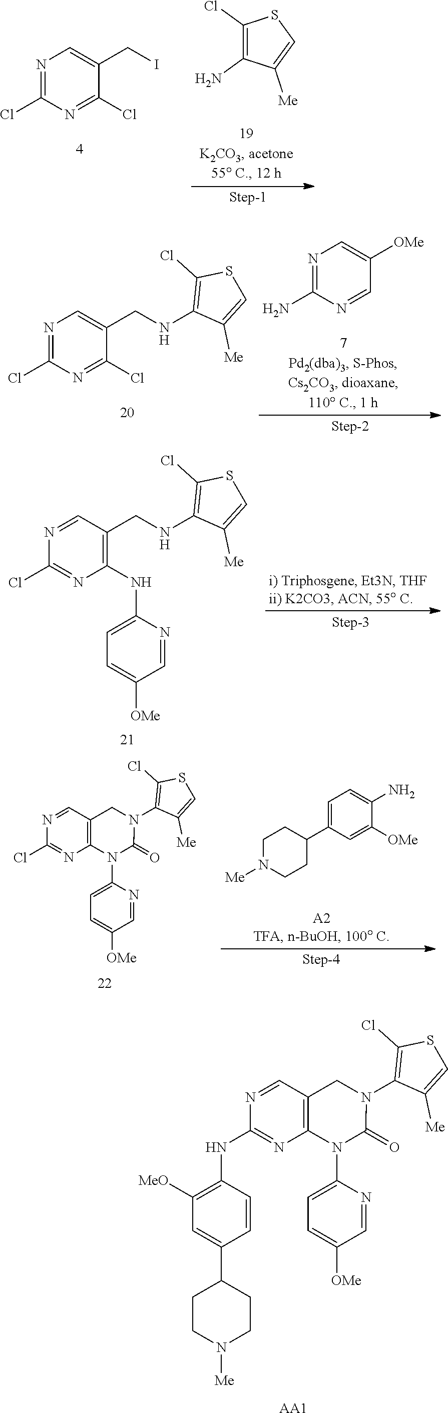

### Step-1: 2-chloro-N-((2,4-dichloropyrimidin-5-yl)methyl)-4-methylthiophen-3-amine (20)

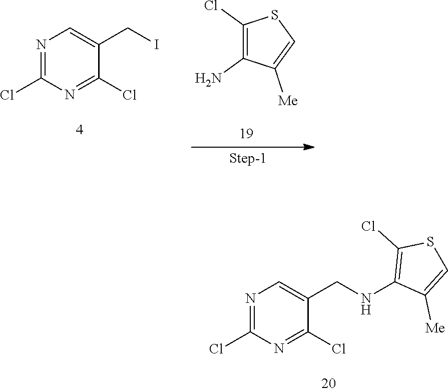 [SMILES: Clc1ncc(CI)c(Cl)n1 ; Cc1csc(Cl)c1N ; Cc1csc(Cl)c1NCc1cnc(Cl)nc1Cl]

To a solution of 2,4-dichloro-5-(iodomethyl)pyrimidine (4) (5.0 g, 17.36 mmol 1.0 eq.) and 2-chloro-4-methylthiophen-3-amine (19) (3.3 g, 22.44 mmol 1.3 eq.) in Acetone (30 mL) was added K₂CO₃ (7.2 g, 52.09 mmol 3.0 eq.) then stirred at 55° c. for 12 h. The progress of the reaction was monitored by TLC. The TLC shows complete consumption of starting material. The reaction mixture was filtered and the filtrate was concentrated to get crude compound. The residue was purified by column chromatography on silica gel to afford 2-chloro-N-((2,4-dichloropyrimidin-5-yl)methyl)-4-methylthiophen-3-amine (20) (2.5 g, 47.16%) as a gummy solid. LCMS: m/z=307.97 [M+H]⁺, 96.31% (8.42 min).

### Step-2: 2-chloro-5-((2-chloro-4-methylthiophen-3-ylamino) methyl)-N-(5-methoxypyridin-2-yl) pyrimidin-4-amine (21)

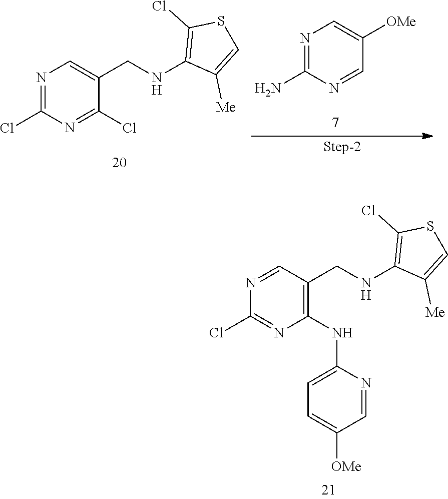 [SMILES: Cc1csc(Cl)c1NCc1cnc(Cl)nc1Cl ; COc1cnc(N)nc1 ; COc1ccc(Nc2nc(Cl)ncc2CNc2c(C)csc2Cl)nc1]

A mixture of 2-chloro-N-((2,4-dichloropyrimidin-5-yl)methyl)-6-methylaniline (20) (2.5 g, 8.14 mmol 1.0 eq.), 5-methoxypyridin-2-amine (7) (1.22 g, 9.76 mmol 1.2 eq.), Cs₂CO₃ (7.7 g, 23.63 mmol 3.0 eq.) in dioxane (20 mL) was degassed for 10 min, to a reaction mixture was added Pd₂(dba)₃ (0.74 g, 0.80 mmol 0.1 eq.) and S-Phos (0.66 g, 1.60 mmol 0.2 eq.) and heated at 110° C. for 4 h. The progress of the reaction was monitored by TLC. The TLC shows complete consumption of starting material. After which it was filtered with a pad of celite and concentrated under reduced pressure. The residue was purified by column chromatography on silica gel to afford 2-chloro-5-((2-chloro-4-methylthiophen-3-ylamino) methyl)-N-(5-methoxypyridin-2-yl) pyrimidin-4-amine (21) (0.6 g, 18.7%) as a off-gummy liquid. LCMS: m/z=396.05 [M+H]⁺, 92.05% (8.50 min).

### Step-3: 7-chloro-3-(2-chloro-4-methylthiophen-3-yl)-1-(5-methoxypyridin-2-yl)-3,4-dihydropyrimido[4,5-d]pyrimidin-2(1H)-one (22)

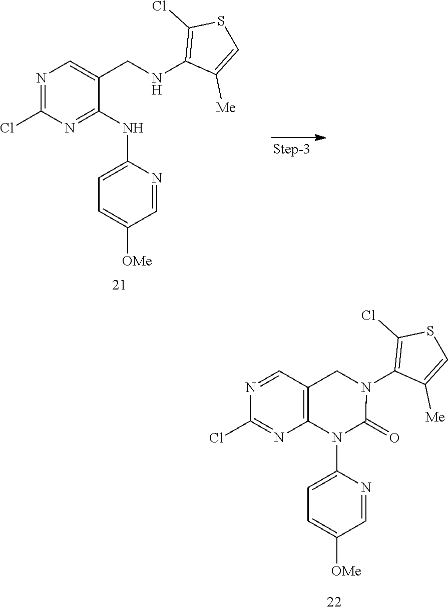 [SMILES: COc1ccc(Nc2nc(Cl)ncc2CNc2c(C)csc2Cl)nc1 ; COc1ccc(N2C(=O)N(c3c(C)csc3Cl)Cc3cnc(Cl)nc32)nc1]

To a solution of 2-chloro-5-((2-chloro-4-methylthiophen-3-ylamino)methyl)-N-(5-methoxypyridin-2-yl)pyrimidin-4-amine (21) (0.6 g, 1.51 mmol 1.0 eq.) in Dry THF (5.0 mL) Triphosgene (490 mg, 1.651 mmol 1.1 eq.) in THF (1 mL) were added followed by Et₃N (184 mg, 1.818 mmol 1.2 eq.) added, then stirred at Room temperature for 2 h, to the reaction mixture K₂CO₃ (2.1 g 15.19 mmol 10.0 eq.) and ACN (2 mL) were added, then stirred at 55° C. for 16 h. The progress of the reaction was monitored by TLC. The TLC shows complete consumption of starting material. The reaction mixture was filtered and the filtrate was concentrated to get crude compound. The residue was purified by column chromatography on silica gel to afford 7-chloro-3-(2-chloro-4-methylthiophen-3-yl)-1-(5-methoxypyridin-2-yl)-3,4-dihydropyrimido[4,5-d]pyrimidin-2(1H)-one (22) (0.3 g, 47.61%) as a pale yellow solid. LCMS: m/z=422.01 [M+H]⁺, 96.74% (6.42 min).

### Step-4: 3-(2-chloro-4-methylthiophen-3-yl)-7-(2-methoxy-4-(1-methylpiperidin-4-yl) phenylamino)-1-(5-methoxypyridin-2-yl)-3,4-dihydropyrimido[4,5-d]pyrimidin-2(1H)-one (AA1)

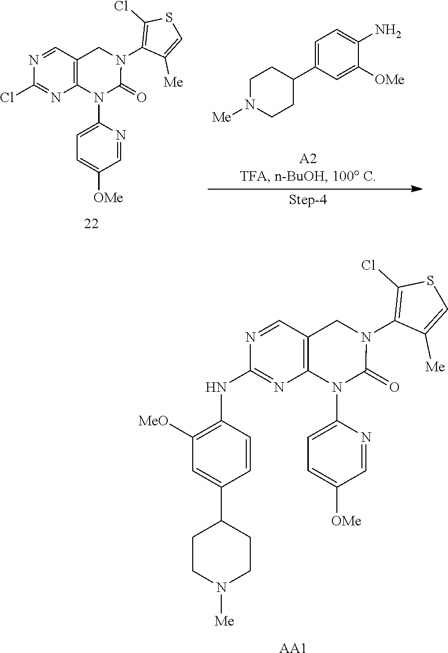

To a solution of 7-chloro-3-(2-chloro-4-methylthiophen-3-yl)-1-(5-methoxypyridin-2-yl)-3,4-dihydropyrimido[4,5-d]pyrimidin-2(1H)-one (22) (300 mg, 0.712 mmol 1.0 eq.) and 2-methoxy-4-(1-methylpiperidin-4-yl)aniline (A2) (0.19 g 0.863 mmol 1.2 eq) in n-BuOH (2 mL) was added Trifluroacetic acid (0.108 g, 0.877 mmol 1.3 eq.) and stirred at 100° C. for 6 h. The progress of the reaction was monitored by TLC. The TLC shows complete consumption of starting material The reaction mixture was concentrated and was purified with Combiflash Rf with Teledyne IscoRediSepRfHigh Performance Gold or SilicycleSiliaSep High Performance columns (40, 80, or 120 g) to afford 3-(2-chloro-4-methylthiophen-3-yl)-7-(2-methoxy-4-(1-methylpiperidin-4-yl)phenylamino)-1-(5-methoxypyridin-2-yl)-3,4-dihydropyrimido[4,5-d]pyrimidin-2(1H)-one (AA1) (100 mg, 23%) as a white solid. Analytical data for this synthesised compound AA1 is shown in Table 1.1.

### Compound AA2

### Synthesis of 3-(2-chloro-4-(difluoromethyl) thiophen-3-yl)-1-(5-methoxypyridin-2-yl)-7-(4-(1-methylpiperidin-4-yl) phenylamino)-3,4-dihydropyrimido[4,5-d]pyrimidin-2(1H)-one (AA2)

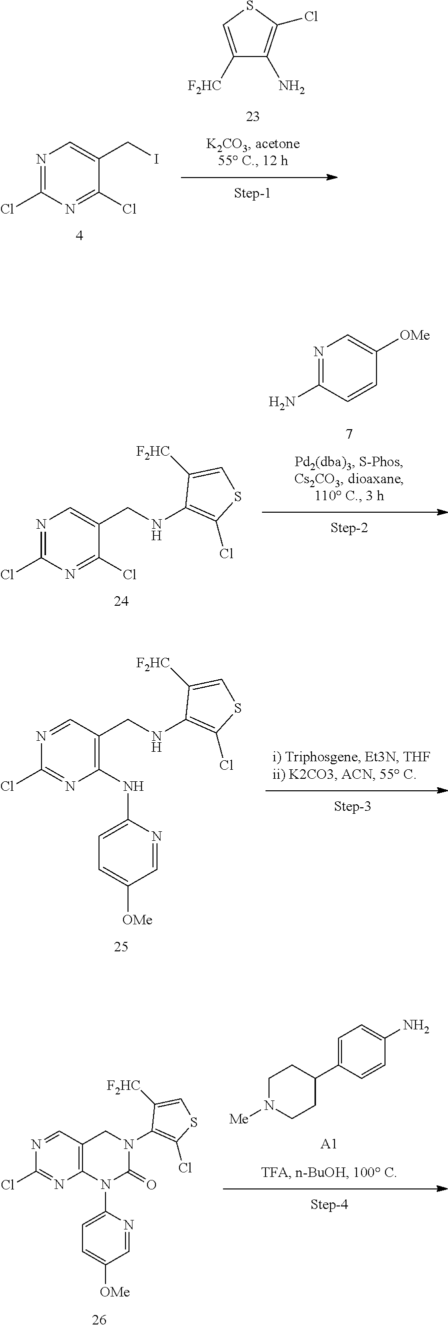

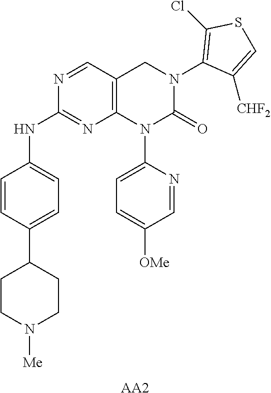

### Step-1: 2-chloro-N-((2,4-dichloropyrimidin-5-yl)methyl)-4-(difluoromethyl)thiophen-3-amine (24)

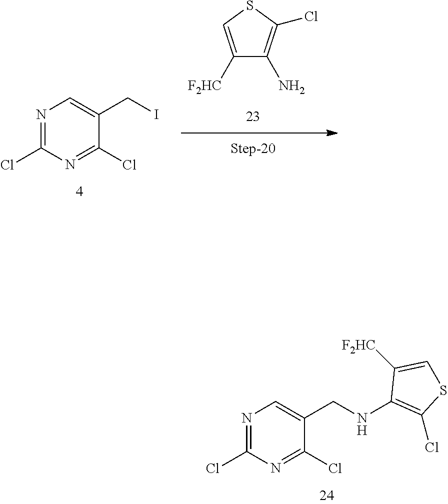 [SMILES: Nc1c(C(F)F)csc1Cl ; Clc1ncc(CI)c(Cl)n1 ; FC(F)c1csc(Cl)c1NCc1cnc(Cl)nc1Cl]

To a solution of 2,4-dichloro-5-(iodomethyl)pyrimidine (4) (5.0 g, 17.36 mmol 1.0 eq.) and 2-chloro-4-(difluoromethyl)thiophen-3-amine (23) (4.1 g, 22.40 mmol 1.3 eq.) in Acetone (30 mL) was added K₂CO₃ (7.2 g, 52.09 mmol 3.0 eq.) then stirred at 55° c. for 12 h. The progress of the reaction was monitored by TLC. The TLC shows complete consumption of starting material. The reaction mixture was filtered and the filtrate was concentrated to get crude compound. The residue was purified by column chromatography on silica gel to afford 2-chloro-N-((2,4-dichloropyrimidin-5-yl)methyl)-6-methylaniline (24) (2.1 g, 35.59%) as a gummy liquid. LCMS: m/z=343.89 [M+H]⁺, 79.84% (4.11 min).

### Step-2: 2-chloro-5-((2-chloro-4-(difluoromethyl) thiophen-3-ylamino) methyl)-N-(5-methoxypyridin-2-yl) pyrimidin-4-amine (25)

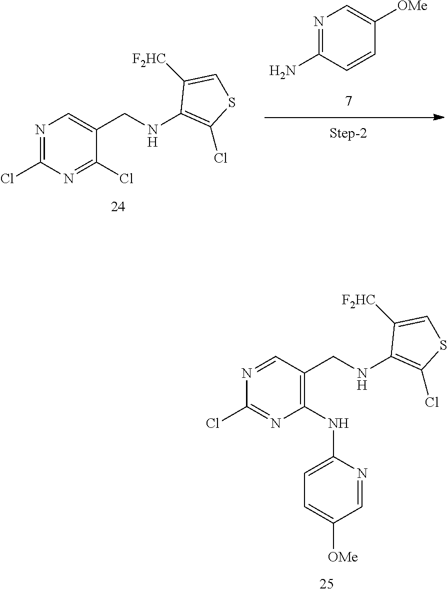 [SMILES: FC(F)c1csc(Cl)c1NCc1cnc(Cl)nc1Cl ; COc1ccc(N)nc1 ; COc1ccc(Nc2nc(Cl)ncc2CNc2c(C(F)F)csc2Cl)nc1]

A mixture of 2-chloro-N-((2,4-dichloropyrimidin-5-yl)methyl)-6-methylaniline (24) (2.0 g, 6.155 mmol 1.0 eq.), 5-methoxypyridin-2-amine (7) (0.87 g, 6.96 mmol 1.2 eq.), Cs₂CO₃ (5.6 g, 17.18 mmol 3.0 eq.) in dioxane (20 mL) was degassed for 10 min, to a reaction mixture was added Pd₂(dba)₃ (0.53 g, 0.615 mmol 0.1 eq.) and S-Phos (0.47 g, 0.223 mmol 0.2 eq.) and heated at 110° C. for 3 h. The progress of the reaction was monitored by TLC. The TLC shows complete consumption of starting material. After which it was filtered with a pad of celite and concentrated under reduced pressure. The residue was purified by column chromatography on silica gel to afford 2-chloro-5-((2-chloro-4-(difiluoromethyl) thiophen-3-ylamino) methyl)-N-(5-methoxypyridin-2-yl) pyrimidin-4-amine (25) (0.5 g, 15.5%) as a off-white solid. LCMS: m/z=432.07 [M+H]⁺, 73.24% (4.08 min).

### Step-3: 7-chloro-3-(2-chloro-4-(difluoromethyl) thiophen-3-yl)-1-(5-methoxypyridin-2-yl)-3,4-dihydropyrimido[4,5-d]pyrimidin-2(1H)-one (26)

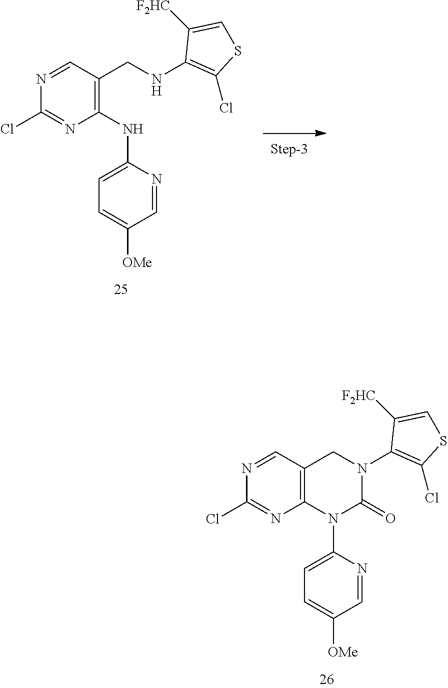 [SMILES: COc1ccc(Nc2nc(Cl)ncc2CNc2c(C(F)F)csc2Cl)nc1 ; COc1ccc(N2C(=O)N(c3c(C(F)F)csc3Cl)Cc3cnc(Cl)nc32)nc1]

To a solution of 2-chloro-5-((2-chloro-6-methylphenylamino)methyl)-N-(5-methoxypyridin-2-yl)pyrimidin-4-amine (25) (0.5 g, 1.106 mmol 1.0 eq.) in DryTHF (5.0 mL) Triphosgene (0.37 g, 1.246 mmol 1.1 eq.) in THF (1 mL) were added followed by Et₃N (0.14 g, 1.382 mmol 1.2 eq.) added, then stirred at Room temperature for 2 h, To the reaction mixture K₂CO₃ (1.6 g 11.57 mmol 10.0 eq.) and ACN (2 mL) were added, then stirred at 55° C. for 16 h. The progress of the reaction was monitored by TLC. The TLC shows complete consumption of starting material. The reaction mixture was filtered and the filtrate was concentrated to get crude compound. The residue was purified by column chromatography on silica gel to afford 7-chloro-3-(2-chloro-4-(difluoromethyl) thiophen-3-yl)-1-(5-methoxypyridin-2-yl)-3,4-dihydropyrimido[4,5-d]pyrimidin-2(1H)-one (26) (0.25 g, 50.25%) as a gummy liquid. LCMS: m/z=458.20 [M+H]⁺, 79.99% (3.38 min).

### Step-4: 3-(2-chloro-4-(difluoromethyl) thiophen-3-yl)-1-(5-methoxypyridin-2-yl)-7-(4-(1-methylpiperidin-4-yl) phenyl amino)-3,4-dihydropyrimido[4,5-d]pyrimidin-2(1H)-one (AA2)

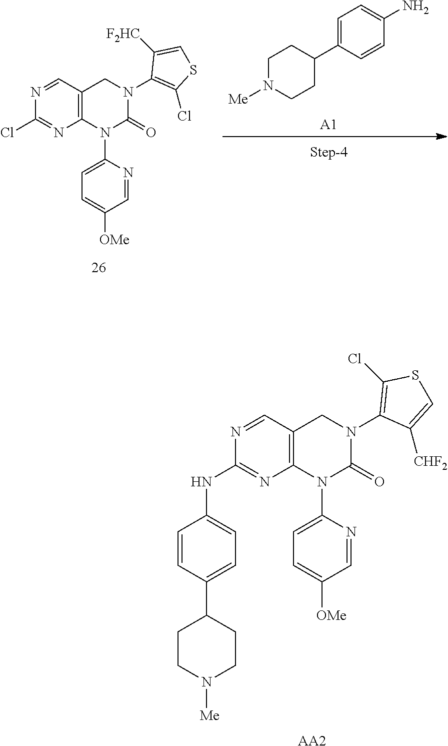

To a solution of 7-chloro-3-(2-chloro-4-(difluoromethyl)thiophen-3-yl)-1-(5-methoxypyridin-2-yl)-3,4-dihydropyrimido[4,5-d]pyrimidin-2(1H)-one (26) (0.25 g, 0.545 mmol 1.0 eq.) and 4-(1-methylpiperidin-4-yl)aniline (A1) (0.12 g, 0.631 mmol 1.2 eq) in n-BuOH (2 mL) was added Trifluroacetic acid (0.12 g, 1.092 mmol, 2.0 eq.) and stirred at 100° C. for 6 h. The progress of the reaction was monitored by TLC. The TLC shows complete consumption of starting material The reaction mixture was concentrated and was purified with Combiflash Rf with Teledyne IscoRediSepRfHigh Performance Gold or SilicycleSiliaSep High Performance columns (40, 80, or 120 g) to afford 3-(2-chloro-4-(difluoromethyl)thiophen-3-yl)-1-(5-methoxypyridin-2-yl)-7-(4-(1-methylpiperidin-4-yl)phenylamino)-3,4-dihydropyrimido[4,5-d]pyrimidin-2(1H)-one (AA2) (100 mg, 30.3%) as a white solid. Analytical data for this synthesised compound AA2 is shown in Table 1.1.

### Compound AA3

### Synthesis of 3-(2-chloro-4-(fluoromethyl) thiophen-3-yl)-1-(5-methoxypyridin-2-yl)-7-(4-(1-methylpiperidin-4-yl) phenyl amino)-3,4-dihydropyrimido[4,5-d]pyrimidin-2(1H)-one (AA3)

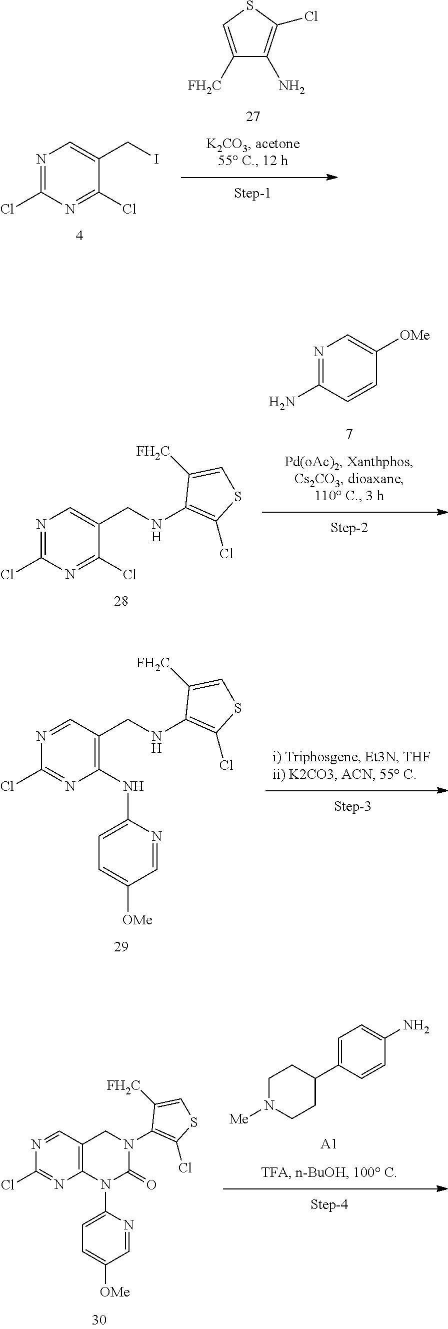

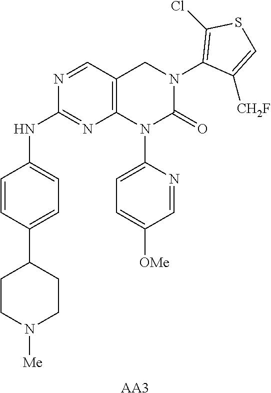

### Step-1: 2-chloro-N-((2,4-dichloropyrimidin-5-yl)methyl)-4-(fluoromethyl)thiophen-3-amine (28)

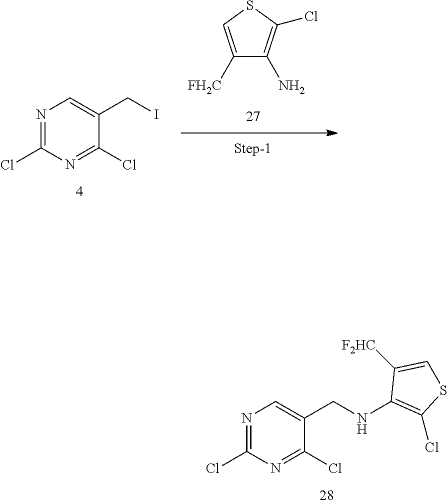

To a solution of 2,4-dichloro-5-(iodomethyl)pyrimidine (4) (5.0 g, 17.36 mmol 1.0 eq.) and 2-chloro-4-(fluoromethyl)thiophen-3-amine (27) (3.7 g, 22.42 mmol 1.3 eq.) in Acetone (30 mL) was added K₂CO₃ (7.2 g, 52.09 mmol 3.0 eq.) then stirred at 55° c. for 12 h. The progress of the reaction was monitored by TLC. The TLC shows complete consumption of starting material. The reaction mixture was filtered and the filtrate was concentrated to get crude compound. The residue was purified by column chromatography on silica gel to afford 2-chloro-N-((2,4-dichloropyrimidin-5-yl)methyl)-6-methylaniline (28) (2.5 g, 44.64%) as a gummy liquid. LCMS: m/z=326.06 [M+H]⁺, 73.47% (4.05 min).

### Step-2: 2-chloro-5-((2-chloro-4-(fluoromethyl) thiophen-3-ylamino) methyl)-N-(5-methoxypyridin-2-yl) pyrimidin-4-amine (29)

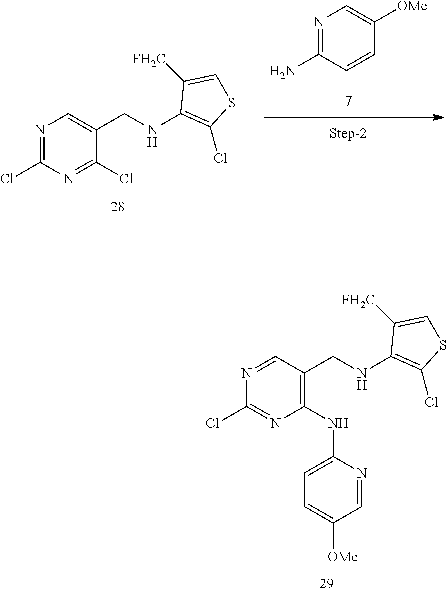

A mixture of 2-chloro-N-((2,4-dichloropyrimidin-5-yl)methyl)-6-methylaniline (28) (2.5 g, 7.692 mmol 1.0 eq.), 5-methoxypyridin-2-amine (7) (1.15 g, 9.20 mmol, 1.2 eq.), Cs₂CO₃ (7.5 g, 23.03 mmol, 3.0 eq.) in dioxane (25 mL) was degassed for 10 min, to a reaction mixture was added Pd(OAc)₂ (0.17 g, 0.757 mmol 0.1 eq.) and Xantphos (0.47 g, 0.223 mmol 0.2 eq.) and heated at 110° C. for 3 h. The progress of the reaction was monitored by TLC. The TLC shows complete consumption of starting material. After which it was filtered with a pad of celite and concentrated under reduced pressure. The residue was purified by column chromatography on silica gel to afford 2-chloro-5-((2-chloro-4-(fluoromethyl) thiophen-3-ylamino) methyl)-N-(5-methoxypyridin-2-yl) pyrimidin-4-amine (29) (0.5 g, 16.12%) as a gummy solid. ¹H NMR (400 MHz, DMSO-d6): δ 9.70 (s, 1H), 8.10-8.08 (m, 2H), 7.96 (d, J=11.6 Hz, 2H), 7.55-7.51 (m, 2H), 5.46-5.23 (t, J=6.8 Hz, 2H), 4.37 (d, J=6.8 Hz, 2H), 3.83 (s, 3H).

### Step-3: 7-chloro-3-(2-chloro-4-(fluoromethyl) thiophen-3-yl)-1-(5-methoxypyridin-2-yl)-3,4-dihydropyrimido[4,5-d]pyrimidin-2(1H)-one (30)

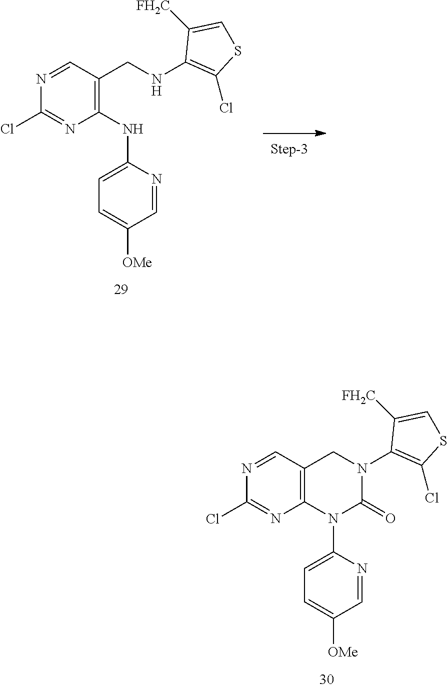

To a solution of 2-chloro-5-((2-chloro-4-(fluoromethyl)thiophen-3-ylamino)methyl)-N-(5-methoxypyridin-2-yl)pyrimidin-4-amine (29) (0.5 g, 1.210 mmol 1.0 eq.) in Dry THF (5.0 mL) Triphosgene (0.37 g, 1.246 mmol 1.1 eq.) in THF (1 mL) were added followed by Et₃N (0.14 g, 1.382 mmol 1.2 eq.) added, then stirred at Room temperature for 2 h, To the reaction mixture K₂CO₃ (1.6 g 11.57 mmol 10.0 eq.) and ACN (2 mL) were added, then stirred at 55° C. for 16 h. The progress of the reaction was monitored by TLC. The TLC shows complete consumption of starting material. The reaction mixture was filtered and the filtrate was concentrated to get crude compound. The residue was purified by column chromatography on silica gel to afford 7-chloro-3-(2-chloro-4-(fluoromethyl)thiophen-3-yl)-1-(5-methoxypyridin-2-yl)-3,4-dihydropyrimido[4,5-d]pyrimidin-2(1H)-one (30) (0.25 g, 47.16%) as a gummy liquid.

### Step-4: 3-(2-chloro-4-(fluoromethyl) thiophen-3-yl)-1-(5-methoxypyridin-2-yl)-7-(4-(1-methylpiperidin-4-yl) phenyl amino)-3,4-dihydropyrimido[4,5-d]pyrimidin-2(1H)-one (AA3)

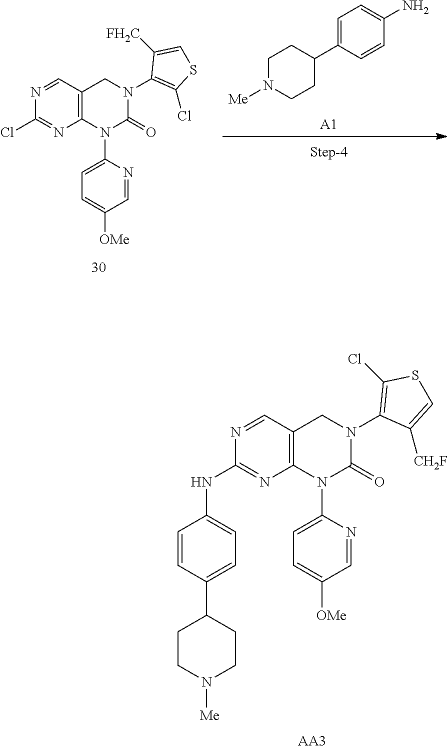

To a solution of 7-chloro-3-(2-chloro-4-(difluoromethyl)thiophen-3-yl)-1-(5-methoxypyridin-2-yl)-3,4-dihydropyrimido[4,5-d]pyrimidin-2(1H)-one (30) (0.25 g, 0.545 mmol 1.0 eq.) and 4-(1-methylpiperidin-4-yl)aniline (A) (0.12 g, 0.631 mmol 1.2 eq) in n-BuOH (2 mL) was added Trifluroacetic acid (0.12 g, 1.092 mmol, 2.0 eq.) and stirred at 100° C. for 6 h. The progress of the reaction was monitored by TLC. The TLC shows complete consumption of starting material The reaction mixture was concentrated and was purified with Combiflash Rf with Teledyne IscoRediSepRfHigh Performance Gold or SilicycleSiliaSep High Performance columns (40, 80, or 120 g) to afford 3-(2-chloro-4-(fluoromethyl)thiophen-3-yl)-1-(5-methoxypyridin-2-yl)-7-(4-(1-methylpiperidin-4-yl)phenylamino)-3,4-dihydropyrimido[4,5-d]pyrimidin-2(1H)-one (AA3) (100 mg, 30.3%) as a white solid. Analytical data for this synthesised compound AA3 is shown in Table 1.1.

### Compound AA4

### Synthesis of 7-(2-methoxy-4-(1-methylpiperidin-4-yl) phenylamino)-1-(5-methoxypyridin-2-yl)-3-(1,3,5-trimethyl-1H-pyrazol-4-yl)-3,4-dihydropyrimido[4,5-d]pyrimidin-2(1H)-one (AA4)

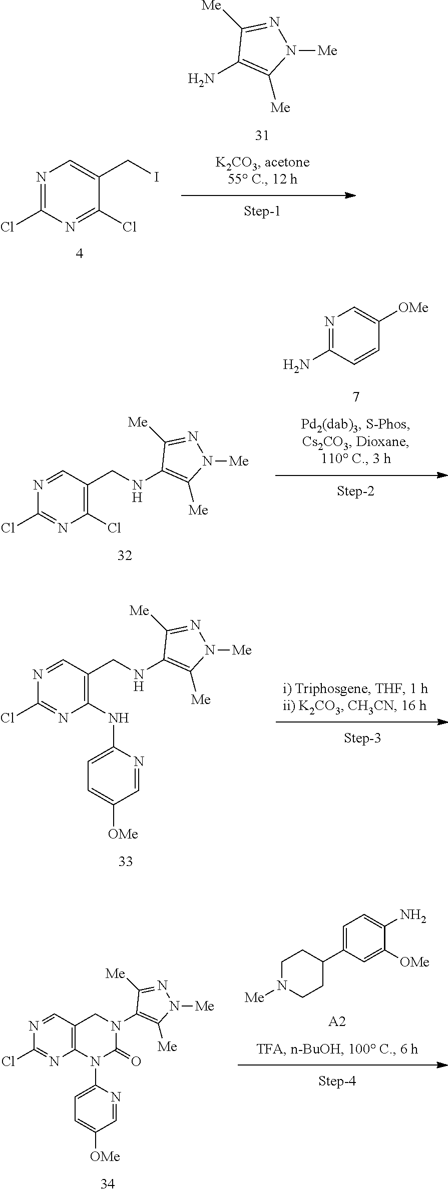

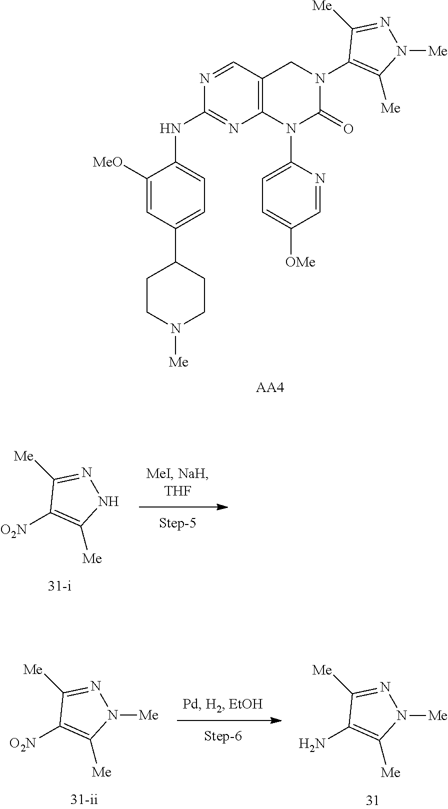

### Step-1: N-((2,4-dichloropyrimidin-5-yl)methyl)-1,3,5-trimethyl-1H-pyrazol-4-amine (32)

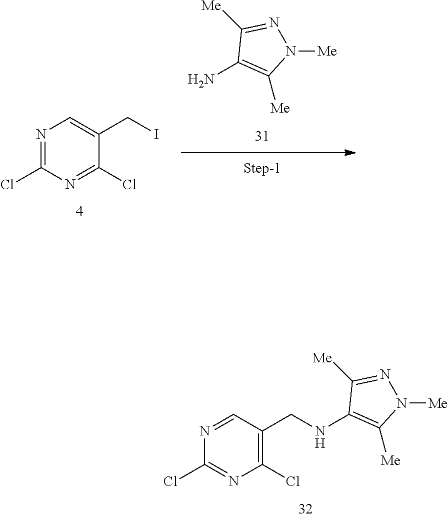 [SMILES: Cc1nn(C)c(C)c1N ; Clc1ncc(CI)c(Cl)n1 ; Cc1nn(C)c(C)c1NCc1cnc(Cl)nc1Cl]

To a solution of 2,4-dichloro-5-(iodomethyl)pyrimidine (4) (5.0 g, 17.36 mmol 1.0 eq.) and 1,3,5-trimethyl-1H-pyrazol-4-amine (31) (2.8 g, 22.40 mmol 1.3 eq.) in Acetone (30 mL) was added K₂CO₃ (7.2 g, 52.09 mmol 3.0 eq.) then stirred at 55° c. for 12 h. The progress of the reaction was monitored by TLC. The TLC shows complete consumption of starting material. The reaction mixture was filtered and the filtrate was concentrated to get crude compound. The residue was purified by column chromatography on silica gel to afford N-((2,4-dichloropyrimidin-5-yl)methyl)-1,3,5-trimethyl-1H-pyrazol-4-amine (32) (2.3 g, 46.93%) as a gummy liquid. LCMS: m/z=286.02 [M+H]⁺, 91.11% (2.46 min).

### Step-2: 2-chloro-N-5-methoxypyridin-2-yl)-5-((1,3,5-trimethyl-1H-pyrazol-4-ylamino)methy)pyrimidin-4-amine (33)

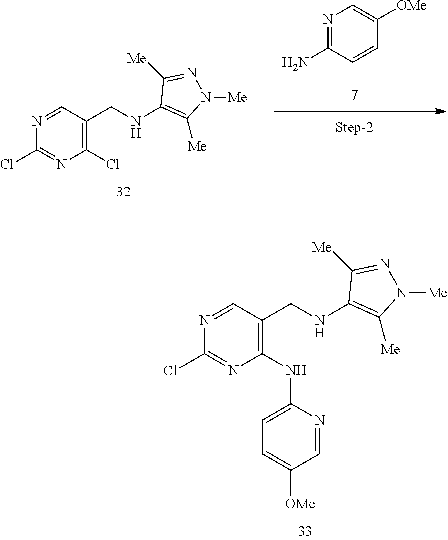 [SMILES: Cc1nn(C)c(C)c1NCc1cnc(Cl)nc1Cl ; COc1ccc(N)nc1 ; COc1ccc(Nc2nc(Cl)ncc2CNc2c(C)nn(C)c2C)nc1]

A mixture of N-((2,4-dichloropyrimidin-5-yl)methyl)-1,3,5-trimethyl-1H-pyrazol-4-amine (32) (2.3 g, 8.070 mmol 1.0 eq.), 5-methoxypyridin-2-amine (7) (0.87 g, 6.96 mmol 1.2 eq.), Cs₂CO₃ (5.6 g, 17.18 mmol 3.0 eq.) in dioxane (20 mL) was degassed for 10 min, to a reaction mixture was added Pd₂(dba)₃ (0.53 g, 0.615 mmol 0.1 eq.) and S-Phos (0.47 g, 0.223 mmol 0.2 eq.) and heated at 100° C. for 3 h. The progress of the reaction was monitored by TLC. The TLC shows complete consumption of starting material. After which it was filtered with a pad of celite and concentrated under reduced pressure. The residue was purified by column chromatography on silica gel to afford 2-chloro-N-(5-methoxypyridin-2-yl)-5-((1,3,5-trimethyl-1H-pyrazol-4-ylamino)methyl)pyrimidin-4-amine (33) (0.4 g, 13.3%) as a gummy liquid. ¹H NMR (400 MHz, DMSO-d₆): δ 10.6 (s, 1H), 8.13-8.11 (m, 1H), 8.08-8.06 (m, 2H), 7.55 (dd, J=3.2 Hz, 1H), 4.33 (m, 1H), 3.95 (d, J=6.0 Hz, 2H), 3.83 (s, 3H), 3.55 (s, 3H), 2.11 (s, 3H), 2.05 (s, 3H). LCMS: m/z=374.16 [M+H]⁺, 94.62% (2.79 min).

### Step-3: 7-chloro-1-(5-methoxypyridin-2-yl)-3-(1,3,5-trimethyl-1H-pyrazol-4-yl)-3,4-dihydropyrimido[4,5-d]pyrimidin-2(1H)-one (34)

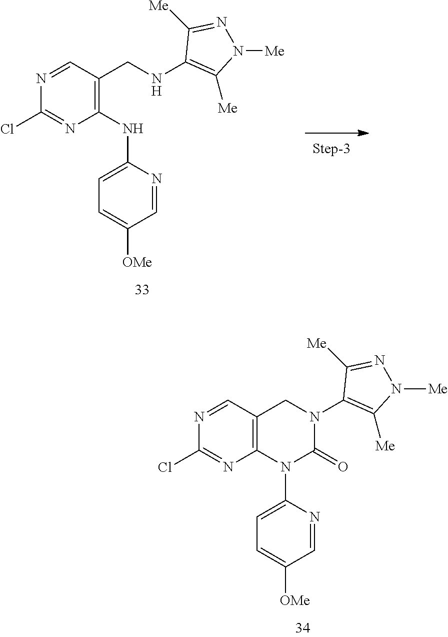 [SMILES: COc1ccc(Nc2nc(Cl)ncc2CNc2c(C)nn(C)c2C)nc1 ; COc1ccc(N2C(=O)N(c3c(C)nn(C)c3C)Cc3cnc(Cl)nc32)nc1]

To a solution of 2-chloro-N-(5-methoxypyridin-2-yl)-5-((1,3,5-trimethyl-1H-pyrazol-4-ylamino)methyl)pyrimidin-4-amine (33) (0.4 g, 1.07 mmol 1.0 eq.) in Dry THF (5.0 mL) Triphosgene (0.35 g, 1.17 mmol 1.1 eq.) in THF (1 mL) were added followed by Et₃N (0.13 g, 1.28 mmol 1.2 eq.) added, then stirred at Room temperature for 2 h, To the reaction mixture K₂CO₃ (1.48 g 11.57 mmol 10.0 eq.) and ACN (2 mL) were added, then stirred at 55° C. for 16 h. The progress of the reaction was monitored by TLC. The TLC shows complete consumption of starting material. The reaction mixture was filtered and the filtrate was concentrated to get crude compound. The residue was purified by column chromatography on silica gel to afford 7-chloro-1-(5-methoxypyridin-2-yl)-3-(1,3,5-trimethyl-1H-pyrazol-4-yl)-3,4-dihydropyrimido[4,5-d]pyrimidin-2(1H)-one (34) (0.2 g, 47.61%) as a gummy liquid. LCMS: m/z=400.26 [M+H]⁺, 60.48% (2.54 min).

### Step-4: 7-(2-methoxy-4-(1-methylpiperidin-4-yl) phenylamino)-1-(5-methoxypyridin-2-yl)-3-(1,3,5-trimethyl-1H-pyrazol-4-yl)-3,4-dihydropyrimido[4,5-d]pyrimidin-2(1H)-one (AA4)

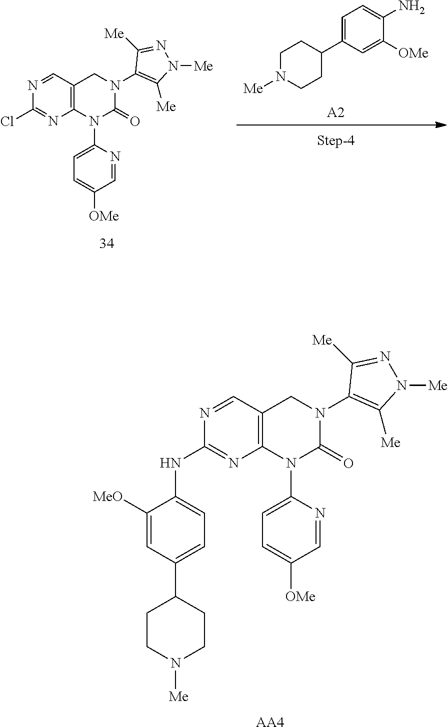

To a solution of 7-chloro-3-(2-chloro-4-(difluoromethyl)thiophen-3-yl)-1-(5-methoxypyridin-2-yl)-3,4-dihydropyrimido[4,5-d]pyrimidin-2(1H)-one (34) (0.2 g, 0.50 mmol 1.0 eq.) and 2-methoxy-4-(1-methylpiperidin-4-yl)aniline (A2) (0.13 g, 0.612 mmol 1.2 eq) in n-BuOH (2 mL) was added Trifluroacetic acid (0.12 g, 1.092 mmol, 2.0 eq.) and stirred at 100° C. for 6 h. The progress of the reaction was monitored by TLC. The TLC shows complete consumption of starting material The reaction mixture was concentrated and was purified with Combiflash Rf with Teledyne IscoRediSepRfHigh Performance Gold or SilicycleSiliaSep High Performance columns (40, 80, or 120 g) to afford 7-(2-methoxy-4-(1-methylpiperidin-4-yl)phenylamino)-1-(5-methoxypyridin-2-yl)-3-(1,3,5-trimethyl-1H-pyrazol-4-yl)-3,4-dihydropyrimido[4,5-d]pyrimidin-2(1H)-one (AA4) (13 mg, 6.89%) as a white solid. Analytical data for this synthesised compound AA4 is shown in Table 1.1.

### Preparation of Intermediate-31

### Step-5: 1,3,5-trimethyl-4-nitro-1H-pyrazole (31-i)

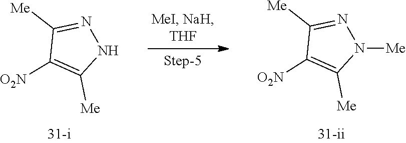 [SMILES: Cc1n[nH]c(C)c1[N+](=O)[O-] ; Cc1nn(C)c(C)c1[N+](=O)[O-]]

To a stirring solution of 3,5-dimethyl-4-nitro-1H-pyrazole (31-i) (5.0 g, 35.46 mmol, 1.0 eq) in anhydrous THF (50 mL) at 0° C., added NaH (60% dispersion in mineral oil, 2.12 g, 53.19 mmol, 1.5 eq), after 15 minutes, remove the ice bath and stirred the reaction mixture for 45 minutes. Added iodomethane (13.0 g, 92.19 mmol, 2.6 eq) to the reaction mixture, then further 3 hours stirred at room temperature, the progress of the reaction was monitored by TLC. The TLC shows complete consumption of starting material. The reaction mixture was quenched with water extracted with EtOAc and the organic layer was washed with brine and dried over MgSO₄, concentrated in vacuo to get the crude residue. The residue was purified by column chromatography on silica gel to afford 1,3,5-trimethyl-4-nitro-1H-pyrazole (31-ii) (5.1 g, 92.8%) as a white solid. LCMS: m/z=156.04 [M+H]⁺, 90.09% (2.54 min).

### Step-6: 1,3,5-trimethyl-1H-pyrazol-4-amine (31)

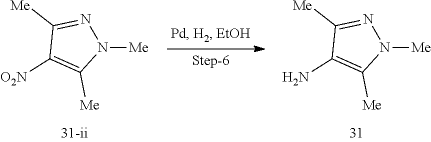 [SMILES: Cc1nn(C)c(C)c1[N+](=O)[O-] ; Cc1nn(C)c(C)c1N]

A mixture of 1,3,5-trimethyl-4-nitro-1H-pyrazole (31-ii) (5.0 g, 29.96 mmol, 1.0 equiv.) and 1.0 g of Pd/C in 10 mL of EtOH (50 mL) was placed under 50 psi of H₂ gas for 16 h. The mixture was filtered and the filtrate was evaporated to afford 1,3,5-trimethyl-1H-pyrazol-4-amine (31) (3.5 g, 87.5%) as a pink solid. ¹H NMR (400 MHz, DMSO-d₆): δ 3.50 (s, 3H), 3.31 (s, 3H), 2.03 (s, 3H), 1.94 (s, 3H). LCMS: m/z=126.00 [M+H]⁺, 87.56% (0.76 min).

### Compound AA5

### Synthesis of 3-(3,5-dimethylisoxazol-4-yl)-7-(2-methoxy-4-(1-methylpiperidin-4-yl)phenylamino)-1-(5-methoxypyridin-2-yl)-3,4-dihydropyrimido[4,5-d]pyrimidin-2(1H)-one (AA5)

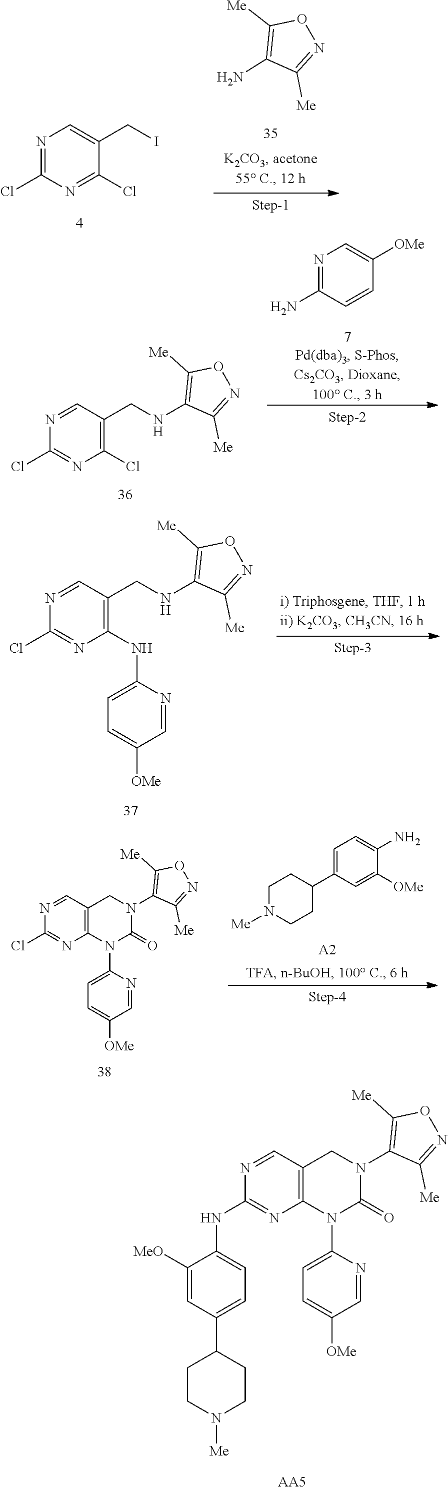

### Step-1: N-((2,4-dichloropyrimidin-5-yl)methyl)-3,5-dimethylisoxazol-4-amine (36)

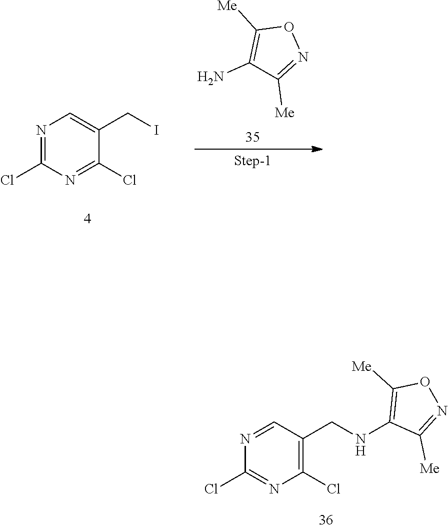 [SMILES: Cc1noc(C)c1N ; Clc1ncc(CI)c(Cl)n1 ; Cc1noc(C)c1NCc1cnc(Cl)nc1Cl]

To a solution of 2,4-dichloro-5-(iodomethyl)pyrimidine (4) (5.0 g, 17.36 mmol 1.0 eq.) and 3,5-dimethylisoxazol-4-amine (35) (2.5 g, 20.66 mmol 1.3 eq.) in Acetone (30 mL) was added K₂CO₃ (7.2 g, 52.09 mmol 3.0 eq.) then stirred at 55° c. for 12 h. The progress of the reaction was monitored by TLC. The TLC shows complete consumption of starting material. The reaction mixture was filtered and the filtrate was concentrated to get crude compound. The residue was purified by column chromatography on silica gel to afford N-((2,4-dichloropyrimidin-5-yl)methyl)-3,5-dimethylisoxazol-4-amine (36) (2.1 g, 46.93%) as a gummy liquid. ¹H NMR (400 MHz, DMSO-d₆): δ 8.78 (s, 1H), 4.54 (t, J=12.0 Hz, 1H), 4.10 (d, J=6.8 Hz, 2H), 2.15 (s, 3H), 2.10 (s, 3H). LCMS: m/z=273.04 [M+H]⁺, 98.56% (3.00 min).

### Step-2: N-((2-chloro-4-(5-methoxypyridin-2-ylamino) pyrimidin-5-yl) methyl)-3,5-dimethylisoxazol-4-amine (37)

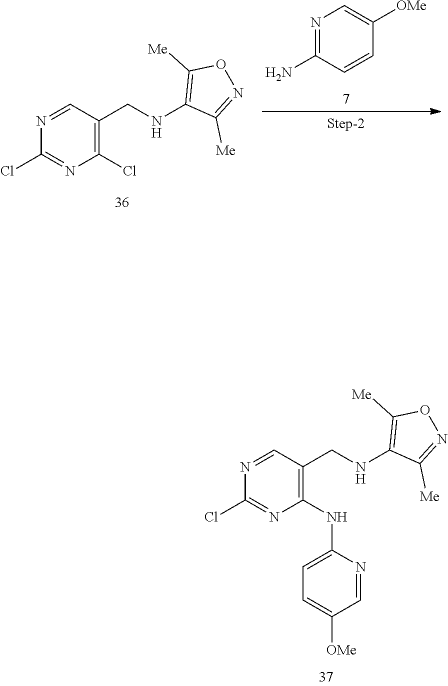 [SMILES: Cc1noc(C)c1NCc1cnc(Cl)nc1Cl ; COc1ccc(N)nc1 ; COc1ccc(Nc2nc(Cl)ncc2CNc2c(C)noc2C)nc1]

A mixture N-((2,4-dichloropyrimidin-5-yl)methyl)-3,5-dimethylisoxazol-4-amine (36) (2.1 g, 7.69 mmol 1.0 eq.), 5-methoxypyridin-2-amine (7) (1.15 g, 9.22 mmol 1.2 eq.), Cs₂CO₃ (7.5 g, 23.07 mmol 3.0 eq.) in dioxane (20 mL) was degassed for 10 min, to a reaction mixture was added Pd₂(dba)₃ (0.71 g, 0.76 mmol 0.1 eq.) and S-Phos (0.63 g, 0.15 mmol 0.2 eq.) and heated at 100° C. for 3 h. The progress of the reaction was monitored by TLC. The TLC shows complete consumption of starting material. After which it was filtered with a pad of celite and concentrated under reduced pressure. The residue was purified by column chromatography on silica gel to afford N-((2-chloro-4-(5-methoxypyridin-2-ylamino) pyrimidin-5-yl) methyl)-3,5-dimethylisoxazol-4-amine (37) (0.3 g, 11.1%) as a gummy liquid. LCMS: m/z=361.10 [M+H]⁺, 84.77% (3.25 min). ¹H NMR (400 MHz, DMSO-d₆): δ 10.0 (s, 1H), 8.14-8.06 (m, 3H), 7.55 (dd, J=12.0 Hz, 1H), 4.65 (m, 1H), 4.09 (d, J=6.0 Hz, 2H), 3.80 (s, 3H), 2.27 (s, 3H), 2.19 (s, 3H).

### Step-3: 7-chloro-3-(3,5-dimethylisoxazol-4-yl)-1-(5-methoxypyridin-2-yl)-3,4-dihydropyrimido[4,5-d]pyrimidin-2(1H)-one-(38)

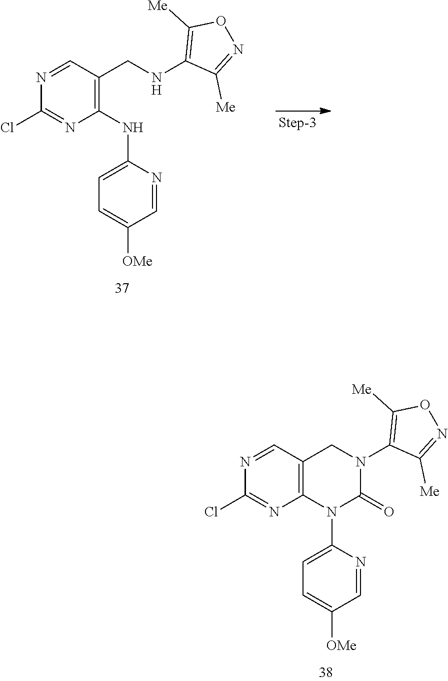 [SMILES: COc1ccc(Nc2nc(Cl)ncc2CNc2c(C)noc2C)nc1 ; COc1ccc(N2C(=O)N(c3c(C)noc3C)Cc3cnc(Cl)nc32)nc1]

To a solution of N-((2-chloro-4-(5-methoxypyridin-2-ylamino)pyrimidin-5-yl)methyl)-3,5-dimethylisoxazol-4-amine (37) (0.3 g, 0.831 mmol 1.0 eq.) in Dry THF (3.0 mL) Triphosgene (0.27 g, 0.91 mmol 1.1 eq.) in THF (1 mL) were added followed by Et₃N (0.11 g, 0.99 mmol 1.2 eq.) added, then stirred at room temperature for 2 h, To the reaction mixture K₂CO₃ (1.14 g 8.301 mmol 10.0 eq.) and ACN (2 mL) were added, then stirred at 55° C. for 16 h. The progress of the reaction was monitored by TLC. The TLC shows complete consumption of starting material. The reaction mixture was filtered and the filtrate was concentrated to get crude compound. The residue was purified by column chromatography on silica gel to 7-chloro-3-(3,5-dimethylisoxazol-4-yl)-1-(5-methoxypyridin-2-yl)-3,4-dihydropyrimido[4,5-d]pyrimidin-2(1H)-one (38) (0.1 g, 31.25%) as a gummy liquid. LCMS: m/z=387.11 [M+H]⁺, 77.73% (2.59 min).

### Step-4: 3-(3,5-dimethylisoxazol-4-yl)-7-(2-methoxy-4-(1-methylpiperidin-4-yl)phenylamino)-1-(5-methoxypyridin-2-yl)-3,4-dihydropyrimido[4,5-d]pyrimidin-2(1H)-one (AA5)

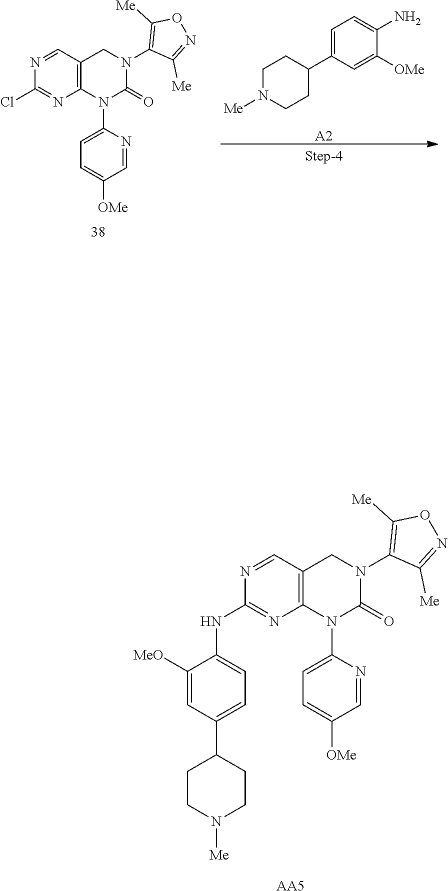

To a solution of 7-chloro-3-(3,5-dimethylisoxazol-4-yl)-1-(5-methoxypyridin-2-yl)-3,4-dihydropyrimido[4,5-d]pyrimidin-2(1H)-one (38) (0.1 g, 0.258 mmol 1.0 eq.) and 2-methoxy-4-(1-methylpiperidin-4-yl)aniline (A2) (0.07 g, 0.318 mmol 1.2 eq) in n-BuOH (1 mL) was added Trifluroacetic acid (0.06 g, 0.516 mmol, 2.0 eq.) and stirred at 100° C. for 6 h. The progress of the reaction was monitored by TLC. The TLC shows complete consumption of starting material The reaction mixture was concentrated and was purified with Combiflash Rf with Teledyne IscoRediSepRfHigh Performance Gold or SilicycleSiliaSep High Performance columns (40, 80, or 120 g) to afford 3-(3,5-dimethylisoxazol-4-yl)-7-(2-methoxy-4-(1-methylpiperidin-4-yl)phenylamino)-1-(5-methoxypyridin-2-yl)-3,4-dihydropyrimido[4,5-d]pyrimidin-2(1H)-one (AA5) (20 mg, 14.2%) as a white solid. Analytical data for this synthesised compound AA5 is shown in Table 1.1.

### Compound AA6

### Synthesis of 3-(2-chloro-4-(fluoromethyl)thiophen-3-yl)-1-(5-methoxypyridin-2-yl)-7-((4-(4-methylpiperazin-1-yl)phenyl)amino)-3,4-dihydropyrimido[4,5-d]pyrimidin-2(1H)-one (AA6)

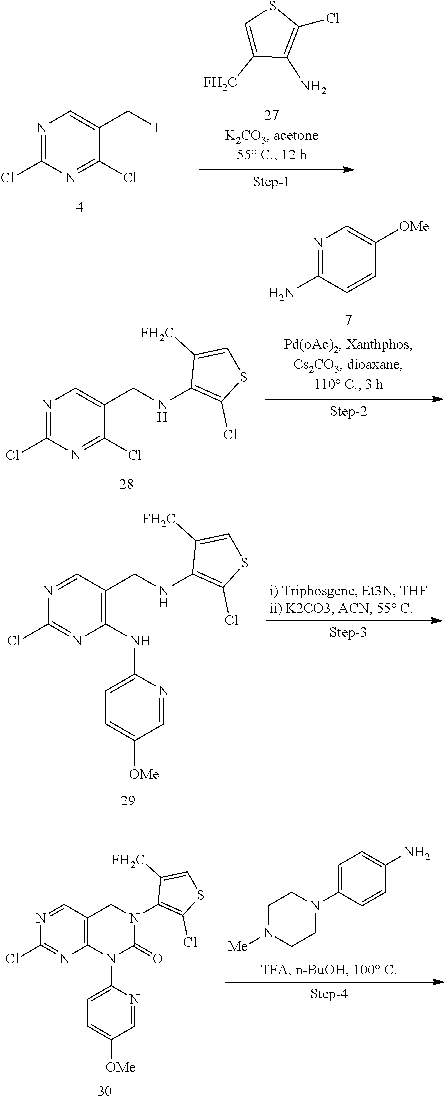

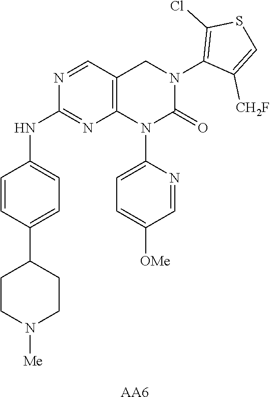

### Step-1: Preparation of Compound 28

The procedure for the preparation of compound 28 was described in step-1 for the synthesis of compound AA3.

### Step-2: Preparation of Compound 29

The procedure for the preparation of compound 29 was described in step-2 for the synthesis of compound AA3.

### Step-3: Preparation of Compound 30

The procedure for the preparation of compound 30 was described in step-3 for the synthesis of compound AA3.

### Step-4: 3-(2-chloro-4-(fluoromethyl)thiophen-3-yl)-1-(5-methoxypyridin-2-yl)-7-((4-(4-methylpiperazin-1-yl)phenyl)amino)-3,4-dihydropyrimido[4,5-d]pyrimidin-2(1H)-one (AA6)

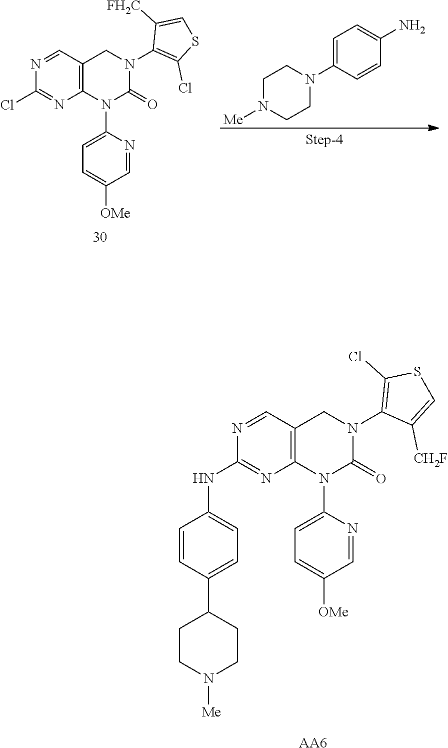

To a solution of 7-chloro-3-(2-chloro-4-(difluoromethyl)thiophen-3-yl)-1-(5-methoxypyridin-2-yl)-3,4-dihydropyrimido[4,5-d]pyrimidin-2(1H)-one (30) (0.1 g, 0.227 mmol 1.0 eq.) and 4-(4-methylpiperazin-1-yl)aniline (0.052 g, 0.272 mmol 1.2 eq) in n-BuOH (2 mL) was added Trifluroacetic acid (0.051 g, 0.454 mmol, 2.0 eq.) and stirred at 100° C. for 6 h. The progress of the reaction was monitored by TLC. The TLC shows complete consumption of starting material The reaction mixture was concentrated and was purified with Combiflash Rf with Teledyne IscoRediSepRfHigh Performance Gold or SilicycleSiliaSep High Performance columns (40, 80, or 120 g) to afford 3-(2-chloro-4-(fluoromethyl)thiophen-3-yl)-1-(5-methoxypyridin-2-yl)-7-((4-(4-methylpiperazin-1-yl)phenyl)amino)-3,4-dihydropyrimido[4,5-d]pyrimidin-2(1H)-one (AA6) (15 mg, 11.5%) as a white solid. Analytical data for this synthesised compound AA6 is shown in Table 1.1.

### Compound AA7

### Synthesis of 3-(2-chloro-4-(fluoromethyl) thiophen-3-yl)-7-(2-methoxy-4-(1-methylpiperidin-4-yl) phenylamino)-1-(5-methoxypyridin-2-yl)-3,4-dihydropyrimido[4,5-d]pyrimidin-2(1H)-one (AA7)

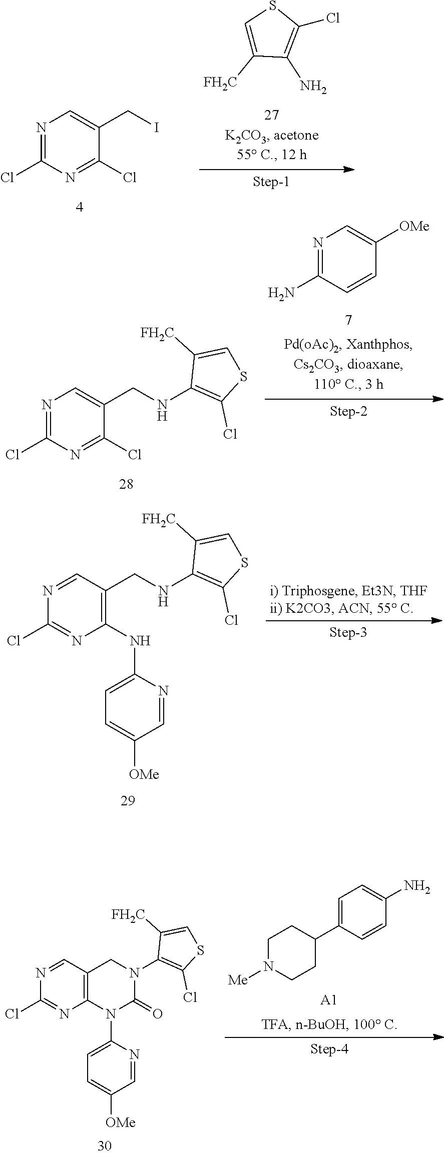

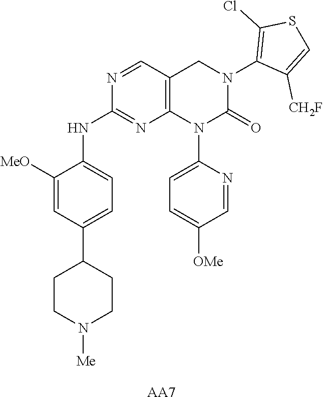

### Step-1: Preparation of Compound 28

The procedure for the preparation of compound 28 was described in step-1 for the synthesis of compound AA3.

### Step-2: Preparation of Compound 29

The procedure for the preparation of compound 29 was described in step-2 for the synthesis of compound AA3.

### Step-3: Preparation of Compound 30

The procedure for the preparation of compound 30 was described in step-3 for the synthesis of compound AA3.

### Step-4: 3-(2-chloro-4-(fluoromethyl) thiophen-3-yl)-7-(2-methoxy-4-(1-methylpiperidin-4-yl) phenylamino)-1-(5-methoxypyridin-2-yl)-3,4-dihydropyrimido[4,5-d]pyrimidin-2(1H)-one (AA7)

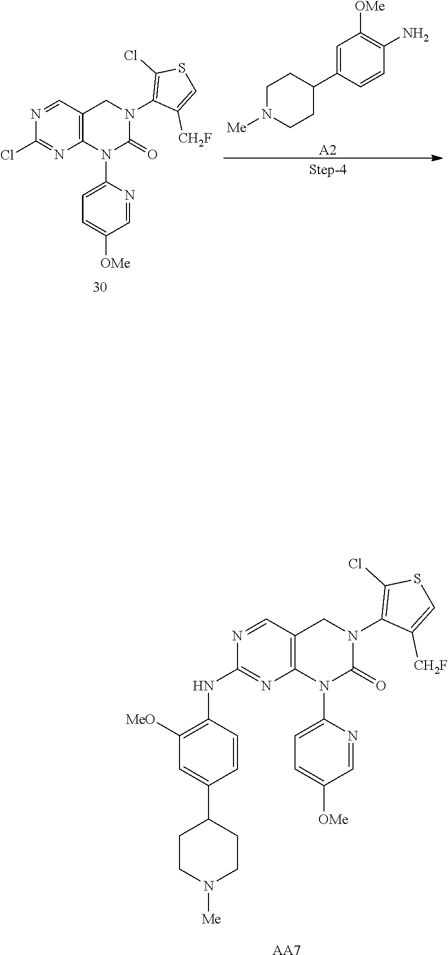

To a solution of 7-chloro-3-(2-chloro-4-(difluoromethyl)thiophen-3-yl)-1-(5-methoxypyridin-2-yl)-3,4-dihydropyrimido[4,5-d]pyrimidin-2(1H)-one (30) (0.12 g, 0.227 mmol 1.0 eq.) and 2-methoxy-4-(1-methylpiperidin-4-yl)aniline (A2) (0.09 g, 0.272 mmol 1.2 eq) in n-BuOH (2 mL) was added Trifluroacetic acid (0.062 g, 0.454 mmol, 2.0 eq.) and stirred at 100° C. for 6 h. The progress of the reaction was monitored by TLC. The TLC shows complete consumption of starting material The reaction mixture was concentrated purified using Combiflash Rf with Teledyne IscoRediSepRfHigh Performance Gold or SilicycleSiliaSep High Performance columns (40, 80, or 120 g) to afford 3-(2-chloro-4-(fluoromethyl)thiophen-3-yl)-7-(2-methoxy-4-(1-methylpiperidin-4-yl)phenylamino)-1-(5-methoxypyridin-2-yl)-3,4-dihydropyrimido[4,5-d]pyrimidin-2(1H)-one (AA7) (15 mg, 11.5%) as a white solid. Analytical data for this synthesised compound AA7 is shown in Table 1.1.

### Compound AA8

### Experimental Procedure of 3-(2-chloro-4-(fluoromethyl) thiophen-3-yl)-1-(5-methoxypyridin-2-yl)-7-(4-(1,2,6-trimethylpiperidin-4-yl)phenylamino)-3,4-dihydropyrimido[4,5-d]pyrimidin-2(1H)-one (AA8)

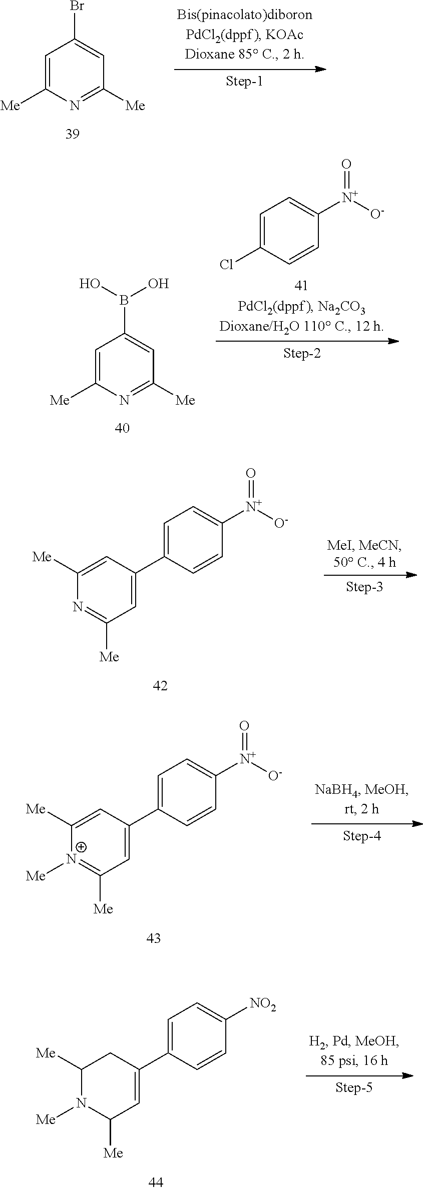 [SMILES: Cc1cc(Br)cc(C)n1 ; O=[N+]([O-])c1ccc(Cl)cc1 ; Cc1cc(B(O)O)cc(C)n1 ; Cc1cc(-c2ccc([N+](=O)[O-])cc2)cc(C)n1 ; Cc1cc(-c2ccc([N+](=O)[O-])cc2)cc(C)[n+]1C ; CC1C=C(c2ccc([N+](=O)[O-])cc2)CC(C)N1C]

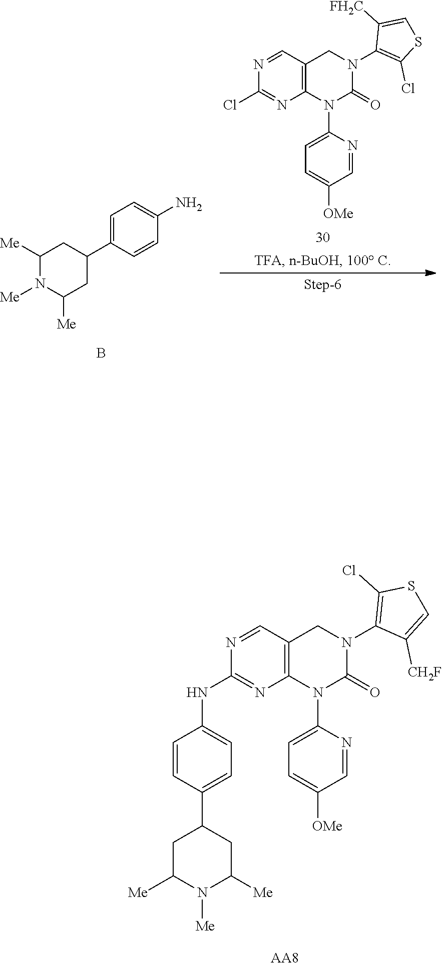

### Step-1: 2,6-dimethylpyridin-4-ylboronic acid (40)

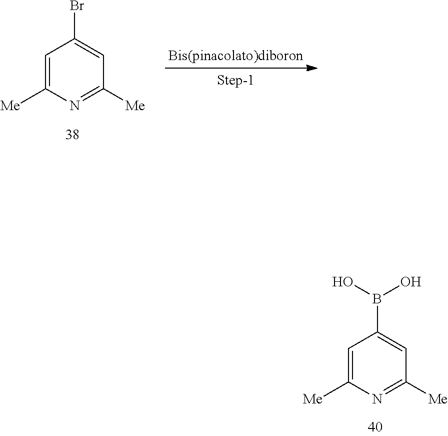 [SMILES: Cc1cc(Br)cc(C)n1 ; Cc1cc(B(O)O)cc(C)n1]

To a mixture of 4-bromo-2,6-dimethylpyridine (39) (5.0 g, 27.02 mmol, 1.0 equiv.), Bis(pinacolato)diboron (8.23 g, 32.42 mmol, 1.2 equiv.), in dioxane (100 mL) KOAc (7.9 g, 81.06 mmol, 3.0 equiv) and Pd(dppf)Cl₂ (1.97 mg, 2.702 mmol, 0.1 equiv.) was added The mixture was stirred for 2 h under N₂ at 85° C. After completion of the reaction, the mixture was cooled to room temperature, filtered, and the filtrate was concentrated to dryness. The residue was diluted with 50 mL of DCM, washed with water, dried over Na₂SO₄ and evaporated to dryness. The residue was purified by silica gel column chromatography to afford 2,6-dimethylpyridin-4-ylboronic acid (40) (3.1 g, 77.5%) as a yellow oil. LCMS: m/z=151.00 [M]⁺, 70.31% (0.86 min).

### Step-2: 2,6-dimethyl-4-(4-nitrophenyl)pyridine (42)

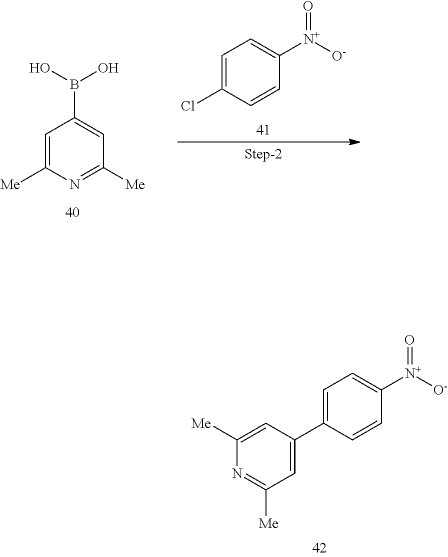 [SMILES: Cc1cc(B(O)O)cc(C)n1 ; O=[N+]([O-])c1ccc(Cl)cc1 ; Cc1cc(-c2ccc([N+](=O)[O-])cc2)cc(C)n1]

To a mixture of 2,6-dimethylpyridin-4-ylboronic acid (40) (3.0 g, 27.02 mmol, 1.0 equiv.), 1-chloro-4-nitrobenzene (41) (3.1 g, 19.74 mmol, 1.0 equiv in dioxane: H2O (3:1, 30 mL) Na₂CO₃ (6.3 g, 59.43 mmol, 3.0 equiv) and Pd(dppf)Cl₂DCM (486 mg, 0.59 mmol, 0.03 equiv.) was added The mixture was stirred for 12 h under N₂ at 110° C. After completion of the reaction, the mixture was cooled to room temperature, filtered, and the filtrate was concentrated to dryness. The residue was diluted with 50 mL of DCM, washed with water, dried over Na₂SO₄ and evaporated to dryness. The residue was purified by silica gel column chromatography to afford 2,6-dimethyl-4-(4-nitrophenyl)pyridine (42) (3.0 g, 66.6%) as a yellow oil. LCMS: m/z=229.08 [M]⁺, 95.40% (2.36 min). ¹H NMR (400 MHz, DMSO-d6): δ 8.32 (d, J=7.0 Hz, 2H), 8.05 (d, J=7.2 Hz, 2H), 7.47 (s, 2H), 2.52-2.49 (m, 6H).

### Step-3: 1,2,6-trimethyl-4-(4-nitrophenyl)pyridinium (43)

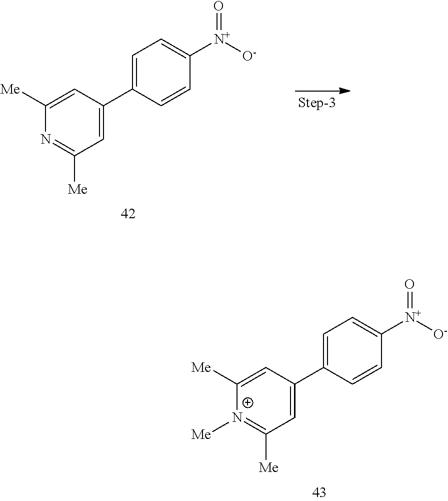 [SMILES: Cc1cc(-c2ccc([N+](=O)[O-])cc2)cc(C)n1 ; Cc1cc(-c2ccc([N+](=O)[O-])cc2)cc(C)[n+]1C]

A mixture of 2,6-dimethyl-4-(4-nitrophenyl)pyridine (42) (3.0 g, 13.15 mmol, 1.0 equiv.) and MeI (6.5 g, 46.02 mmol, 3.5 equiv.) in 30 mL of acetonitrile was stirred for 4 h at 50° C. The solid that formed was collected by filtration, washed with cold acetonitrile, dried in vacuo to afford 1,2,6-trimethyl-4-(4-nitrophenyl)pyridinium (43) (2.9 g, 90%) as a pale yellow solid.

### Step-4: 1,2,6-trimethyl-4-(4-nitrophenyl)-1,2,3,6-tetrahydropyridine (44)

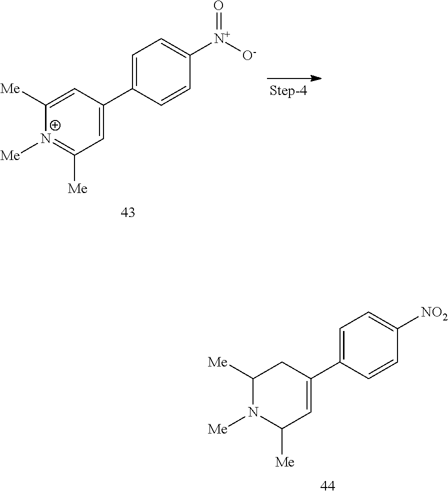 [SMILES: Cc1cc(-c2ccc([N+](=O)[O-])cc2)cc(C)[n+]1C ; CC1C=C(c2ccc([N+](=O)[O-])cc2)CC(C)N1C]

To a solution of 1,2,6-trimethyl-4-(4-nitrophenyl)pyridinium (43) (2.9 g, 11.93 mmol, 1.0 equiv.) in 30 ml o f MeOH was added NaBH₄ (4.5 g, 119.3 mmol, 10.0 equiv.) in portions at 0° C. The mixture was stirred for 2 h at room temperature. The mixture was treated with 40 mL of sat aq NaHCO₃. The solid that formed was collected by filtration and dissolved in 20 mL of 1.0 N HCl, washed with MTBE (2×20 mL). Then the aqueous phase was diluted with sat aq Na₂CO₃, extracted with DCM (3×30 mL), dried over Na₂SO₄ and evaporated to afford 1,2,6-trimethyl-4-(4-nitrophenyl)-1,2,3,6-tetrahydropyridine (44) (1.0 g, 34.12%) as a off-white solid. LCMS: m/z=247.11 [M+H]⁺, 99.1% (2.94, 3.06 min, diastereoisomers).

### Step-5: 4-(1,2,6-trimethylpiperidin-4-yl)aniline (B)

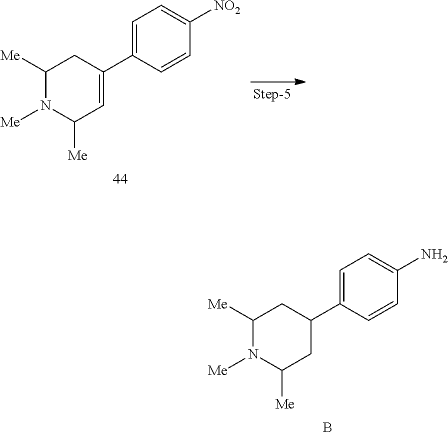

A mixture of 1,2,6-trimethyl-4-(4-nitrophenyl)-1,2,3,6-tetrahydropyridine (44) (1.0 g, 4.065 mmol, 1.0 equiv.) and 0.5 g of Pd/C in 20 mL of MeOH was placed under 85 psi of H₂ gas for 16 h. The mixture was filtered and the filtrate was evaporated to afford 4-(1,2,6-trimethylpiperidin-4-yl)aniline (B) (510 mg 56.8%) as a yellow gummy liquid. LCMS: m/z=219.19 [M+H]⁺.

Preparation Compound-30 procedure was described in the synthesis of compound (AA3).

### Step-6: 3-(2-chloro-4-(fluoromethyl) thiophen-3-yl)-1-(5-methoxypyridin-2-yl)-7-(4-(1,2,6-trimethylpiperidin-4-yl)phenylamino)-3,4-dihydropyrimido[4,5-d]pyrimidin-2(1H)-one (AA8)

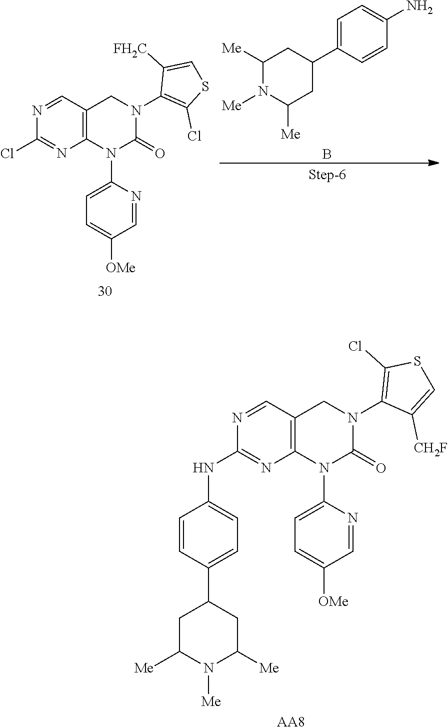

To a solution of 7-chloro-3-(2-chloro-4-(fluoromethyl)thiophen-3-yl)-1-(5-methoxypyridin-2-yl)-3,4-dihydropyrimido[4,5-d]pyrimidin-2(1H)-one (30) (0.1 g, 0.227 mmol 1.0 eq.) and 4-(1,2,6-trimethylpiperidin-4-yl)aniline (B) (0.06 g, 0.272 mmol 1.2 eq) in n-BuOH (2 mL) was added Trifluroacetic acid (0.05 mg, 1.092 mmol, 2.0 eq.) and stirred at 100° C. for 6 h. The progress of the reaction was monitored by TLC. The TLC shows complete consumption of starting material The reaction mixture was concentrated and was purified with Combiflash Rf with Teledyne IscoRediSepRfHigh Performance Gold or SilicycleSiliaSep High Performance columns (40, 80, or 120 g) to afford 3-(2-chloro-4-(fluoromethyl)thiophen-3-yl)-1-(5-methoxypyridin-2-yl)-7-(4-(1,2,6-trimethylpiperidin-4-yl)phenylamino)-3,4-dihydropyrimido[4,5-d]pyrimidin-2(1H)-one (AA8) (20 mg, 14.1%) as a white solid. Analytical data for this synthesised compound AA8 is shown in Table 1.1.

### Compound AA9

### Synthesis of 3-(2-chloro-4-(fluoromethyl) thiophen-3-yl)-7-(4-(1,2-dimethylpiperidin-4-yl)phenylamino)-1-(5-methoxypyridin-2-yl)-3,4-dihydropyrimido[4,5-d]pyrimidin-2(1H)-one (AA9)

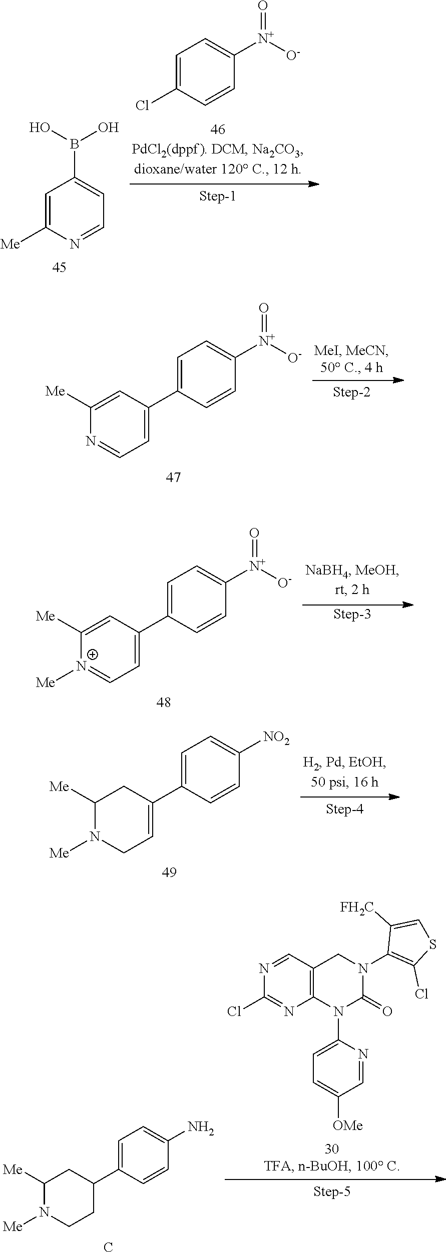

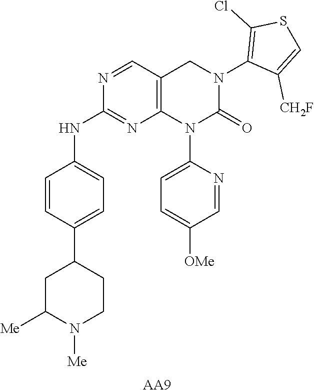

### Step-1: 2-methyl-4-(4-nitrophenyl) Pyridine (47)

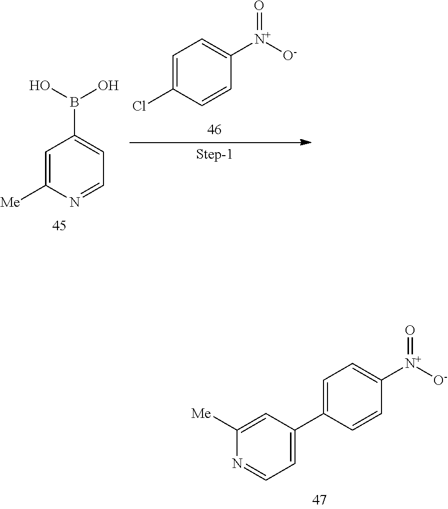

To a mixture of 2-methylpyridin-4-ylboronic acid (45) (2.0 g, 14.60 mmol, 1.0 equiv.) 1-chloro-4-nitrobenzene (46) (2.3 g, 14.60 mmol, 1.0 equiv.), in dioxane: H2O (3:1, 20 mL) Na₂CO₃ (4.6 g, 43.80 mmol, 3.0 equiv) and Pd(dppf)Cl₂ DCM (357 mg, 0.438 mmol, 0.03 equiv.) was added The mixture was stirred for 12 h under N₂ at 120° C. After completion of the reaction, the mixture was cooled to room temperature, filtered, and the filtrate was concentrated to dryness. The residue was diluted with 50 mL of DCM, washed with water, dried over Na₂SO₄ and evaporated to dryness. The residue was purified by silica gel column chromatography to afford 2-methyl-4-(4-nitrophenyl) pyridine (47) (1.7 g, 54.4%) as a white solid. LCMS: m/z=215.02 [M+H]⁺, 94.72% (3.33 min).

### Step-2: 1,2-dimethyl-4-(4-nitrophenyl)pyridinium (48)

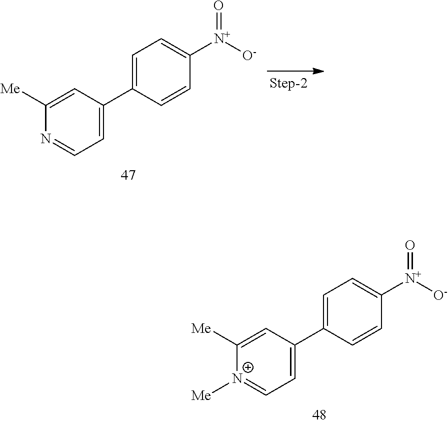 [SMILES: Cc1cc(-c2ccc([N+](=O)[O-])cc2)ccn1 ; Cc1cc(-c2ccc([N+](=O)[O-])cc2)cc[n+]1C]

A mixture of 2-methyl-4-(4-nitrophenyl) pyridine (47) (1.7 g, 7.94 mmol, 1.0 equiv.) and MeI (3.9 g, 27.4 mmol, 3.5 equiv.) in 15 mL of acetonitrile was stirred for 4 h at 50° C. The solid that formed was collected by filtration, washed with cold acetonitrile, dried in vacuo to afford 1,2-dimethyl-4-(4-nitrophenyl)pyridinium (48) (1.6 g, 90%) as a yellow solid.

### Step-3: 1,2-dimethyl-4-(4-nitrophenyl)-1,2,3,6-tetrahydropyridine (49)

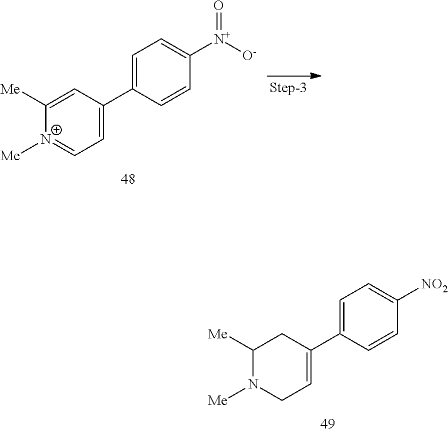 [SMILES: Cc1cc(-c2ccc([N+](=O)[O-])cc2)cc[n+]1C ; CC1CC(c2ccc([N+](=O)[O-])cc2)=CCN1C]

To a solution of 1,2-dimethyl-4-(4-nitrophenyl)pyridinium (48) (1.6 g, 6.98 mmol, 1.0 equiv.) in 20 ml o f MeOH was added NaBH₄ (2.6 g, 69.8 mmol, 10.0 equiv.) in portions at 0° C. The mixture was stirred for 2 h at room temperature. The mixture was treated with 40 mL of sat aq NaHCO₃. The solid that formed was collected by filtration and dissolved in 20 mL of 1.0 N HCl, washed with MTBE (2×20 mL). Then the aqueous phase was diluted with sat aq Na₂CO₃, extracted with DCM (3×30 mL), dried over Na₂SO₄ and evaporated to afford 1,2-dimethyl-4-(4-nitrophenyl)-1,2,3,6-tetrahydropyridine (49) (400 mg, 25.12%) as a pale yellow oil. LCMS: m/z=233.26 [M+H]⁺, 75.21% (6.79 min).

### Step-4;4-(1,2-dimethylpiperidin-4-y)aniline (C)

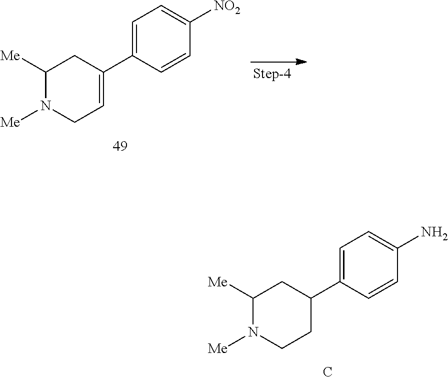

A mixture of 1,2-dimethyl-4-(4-nitrophenyl)-1,2,3,6-tetrahydropyridine (49) (400 mg, 3.88 mmol, 1.0 equiv.) and 0.17 g of Pd/C in 10 mL of MeOH was placed under 80 psi of H₂ gas for 16 h. The mixture was filtered and the filtrate was evaporated to afford 4-(1,2-dimethylpiperidin-4-yl)aniline (C) (0.15 g 44.1%) as a white solid. LCMS: m/z=205.05 [M+H]⁺, 64.13% (4.59 min).

### Step-5: 3-(2-chloro-4-(fluoromethyl) thiophen-3-yl)-7-(4-(1,2-dimethylpiperidin-4-yl)phenylamino)-1-(5-methoxypyridin-2-yl)-3,4-dihydropyrimido[4,5-d]pyrimidin-2(1H)-one (AA9)

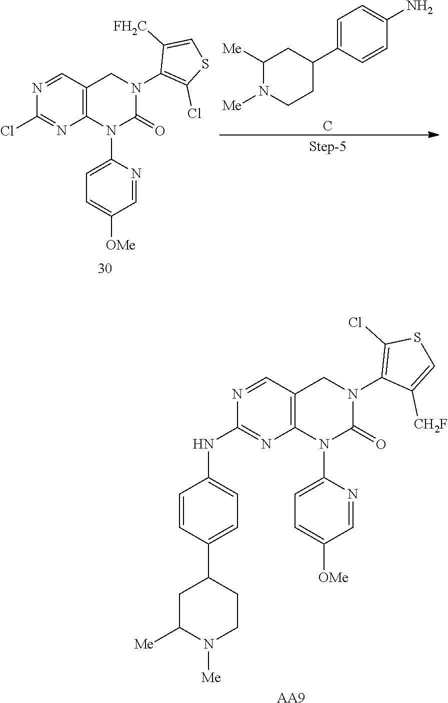

To a solution of 7-chloro-3-(2-chloro-4-(fluoromethyl)thiophen-3-yl)-1-(5-methoxypyridin-2-yl)-3,4-dihydropyrimido[4,5-d]pyrimidin-2(1H)-one (30) (0.1 g, 0.227 mmol 1.0 eq.) and 4-(1,2-dimethylpiperidin-4-yl)aniline (C) (0.07 g, 0.364 mmol 1.5 eq) in n-BuOH (2 mL) was added Trifluroacetic acid (0.05 mg, 0.486 mmol, 2.0 eq.) and stirred at 100° C. for 6 h. The progress of the reaction was monitored by TLC. The TLC shows complete consumption of starting material The reaction mixture was concentrated and was purified with Combiflash Rf with Teledyne IscoRediSepRfHigh Performance Gold or SilicycleSiliaSep High Performance columns (40, 80, or 120 g) to afford 3-(2-chloro-4-(fluoromethyl)thiophen-3-yl)-7-(4-(1,2-dimethylpiperidin-4-yl)phenylamino)-1-(5-methoxypyridin-2-yl)-3,4-dihydropyrimido[4,5-d]pyrimidin-2(1H)-one (AA9) (14 mg, 10.1%) as a white solid. Analytical data for this synthesised compound AA9 is shown in Table 1.1.

### Compound AA10

### Synthesis of 3-(2-chloro-4-(fluoromethyl) thiophen-3-yl)-7-(2-methoxy-4-(1-methylpiperidin-4-yl) phenylamino)-1-(4-methoxyphenyl)-3,4-dihydropyrimido[4,5-d]pyrimidin-2(1H)-one (AA10)

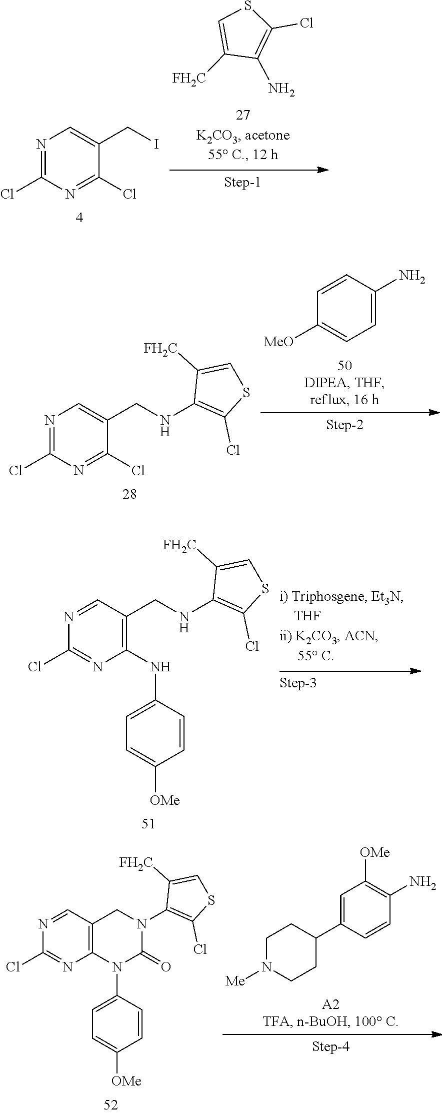

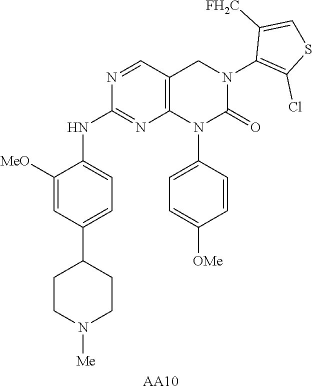

### Step-1: Preparation of Compound 28

The procedure for the preparation of compound 28 was described in step-1 for the synthesis of compound AA3.

### Step-2: 2-chloro-5-((2-chloro-4-(fluoromethyl) thiophen-3-ylamino)methyl)-N-(4-methoxyphenyl)pyrimidin-4-amine (51)

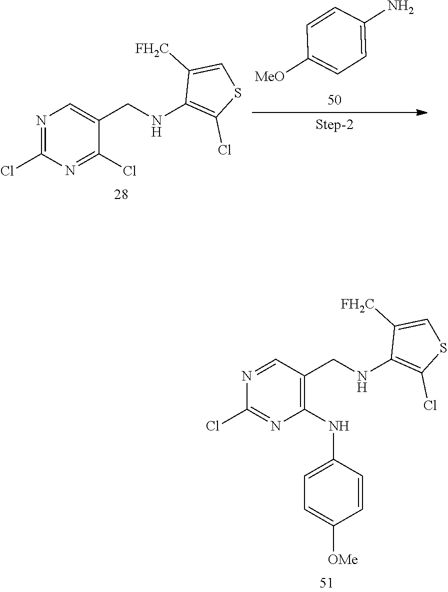

A mixture of 2-chloro-N-((2,4-dichloropyrimidin-5-yl)methyl)-4-(fluoromethyl)thiophen-3-amine (28) (1.0 g, 3.067 mmol 1.0 eq.), 4-methoxyaniline (50) (1.9 g, 15.42 mmol, 5.0 eq) in Dry THF (5 mL) DIPEA (3.17 g, 24.2 mmol, 8.0 eq) were added then heated to reflux for 16 h. The progress of the reaction was monitored by TLC. The TLC shows complete consumption of starting material. After which was concentrated under reduced pressure. The residue was purified by column chromatography on silica gel to afford 2-chloro-5-((2-chloro-4-(fluoromethyl) thiophen-3-ylamino) methyl)-N-(4-methoxyphenyl) pyrimidin-4-amine (51) (0.6 g, 47.6%) as a pale yellow solid.

### Step-3: 7-chloro-3-(2-chloro-4-(fluoromethyl) thiophen-3-yl)-1-(4-methoxyphenyl)-3,4-dihydropyrimido[4,5-d]pyrimidin-2(1H)-one (52)

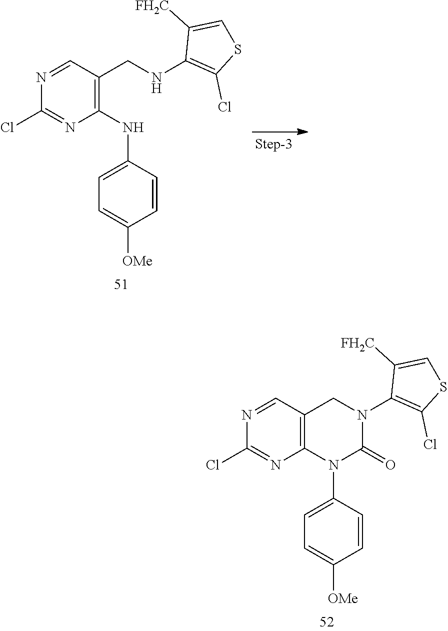

To a solution of 2-chloro-5-((2-chloro-4-(fluoromethyl)thiophen-3-ylamino)methyl)-N-(4-methoxyphenyl)pyrimidin-4-amine (51) (0.21 g, 0.508 mmol, 1.0 eq.) in Dry THF (2.0 mL) Triphosgene (0.17 g, 0.558 mmol, 1.1 eq.) in THF (1 mL) were added followed by Et₃N (0.079 g, 0.725 mmol, 1.5 eq.) added, then stirred at room temperature for 2 h, to the reaction mixture K₂CO₃ (0.70 g, 5.082 mmol, 10.0 eq.) and ACN (1 mL) were added, then stirred at 55° C. for 16 h. The progress of the reaction was monitored by TLC. The TLC shows complete consumption of starting material. The reaction mixture was filtered and the filtrate was concentrated to get crude compound. The residue was purified by column chromatography on silica gel to afford 7-chloro-3-(2-chloro-4-(fluoromethyl) thiophen-3-yl)-1-(4-methoxyphenyl)-3,4-dihydropyrimido[4,5-d]pyrimidin-2(1H)-one (52) (0.13 g, 59.09%) as a gummy liquid. LCMS: m/z=439.01 [M+H]⁺ 47.96% (3.29 min).

### Step-4: 3-(2-chloro-4-(fluoromethyl) thiophen-3-yl)-7-(2-methoxy-4-(1-methylpiperidin-4-yl) phenylamino)-1-(4-methoxyphenyl)-3,4-dihydropyrimido[4,5-d]pyrimidin-2(1H)-one (AA10)

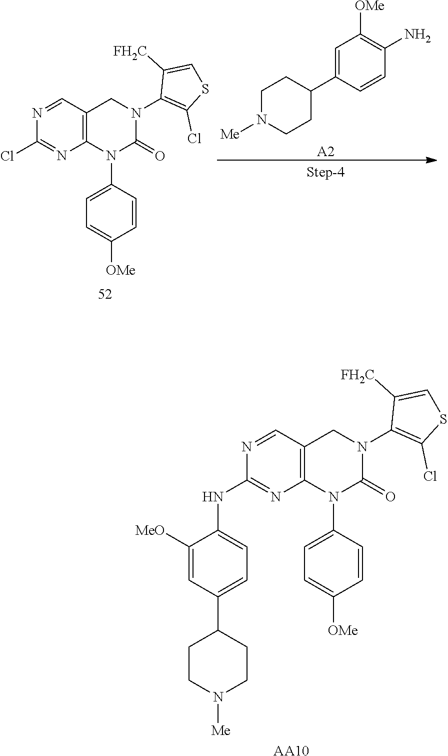

To a solution of 7-chloro-3-(2-chloro-4-(fluoromethyl)thiophen-3-yl)-1-(4-methoxyphenyl)-3,4-dihydropyrimido[4,5-d]pyrimidin-2(1H)-one (52) (0.13 g, 0.296 mmol 1.0 eq.) and 2-methoxy-4-(1-methylpiperidin-4-yl)aniline (A2) (0.1 g, 0.445 mmol 1.5 eq) in n-BuOH (2 mL) was added Trifluroacetic acid (0.07 mg, 0.593 mmol, 2.0 eq.) and stirred at 100° C. for 6 h. The progress of the reaction was monitored by TLC. The TLC shows complete consumption of starting material The reaction mixture was concentrated and was purified with Combiflash Rf with Teledyne IscoRediSepRfHigh Performance Gold or SilicycleSiliaSep High Performance columns (40, 80, or 120 g) to afford 3-(2-chloro-4-(fluoromethyl) thiophen-3-yl)-7-(2-methoxy-4-(1-methylpiperidin-4-yl)phenylamino)-1-(4-methoxyphenyl)-3,4-dihydropyrimido[4,5-d]pyrimidin-2(1H)-one (AA10) (13 mg, 7.6%) as a white solid. Analytical data for this synthesised compound AA10 is shown in Table 1.1.

### Compound AA11

### Synthesis of 3-(2-chloro-4-(fluoromethyl) thiophen-3-yl)-7-(2-methoxy-4-(1-methylpiperidin-4-yl) phenylamino)-1-methyl-3,4-dihydropyrimido[4,5-d]pyrimidin-2(1H)-one (AA11)

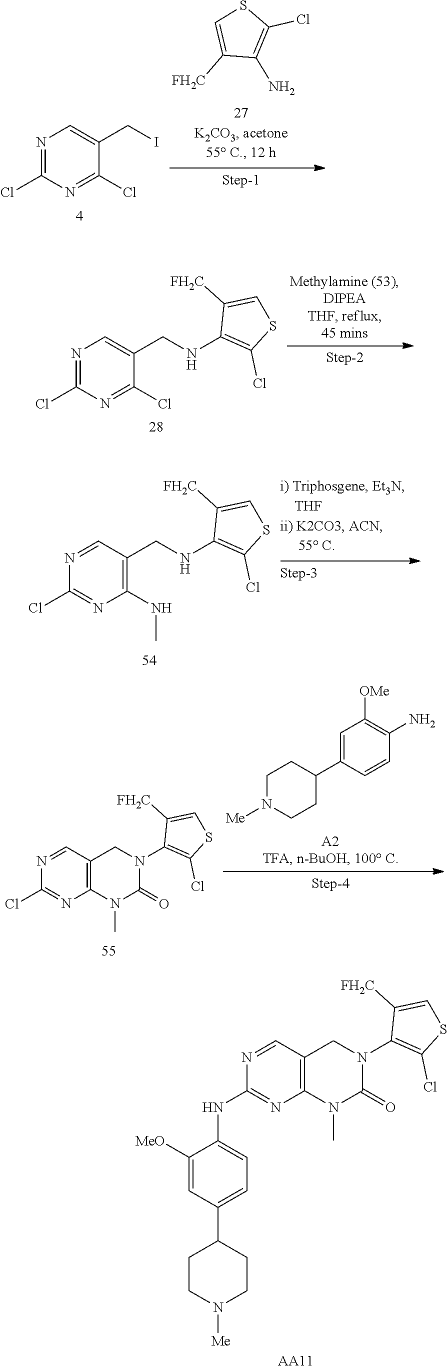

### Step-1: Preparation of Compound 28

The procedure for the preparation of compound 28 was described in step-1 for the synthesis of compound AA3.

### Step-2: 2-chloro-5-((2-chloro-4-(fluoromethyl) thiophen-3-ylamino) methyl)-N-methylpyrimidin-4-amine (54)

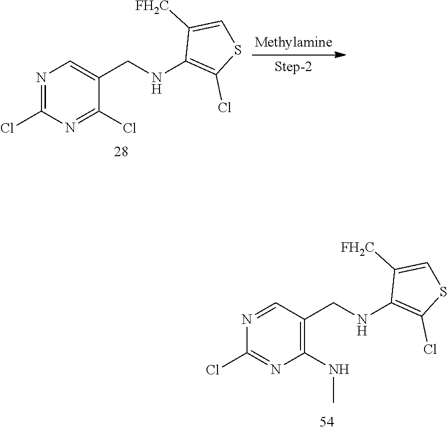

A mixture of 2-chloro-N-((2,4-dichloropyrimidin-5-yl)methyl)-4-(fluoromethyl)thiophen-3-amine (28) (0.5 g, 1.538 mmol 1.0 eq.), methylamine (53) (1.0M soln in THF) (0.28 g, 7.69 mmol, 5.0 eq) in Dry THF (5 mL) DIPEA (0.99 g, 12.30 mmol, 8.0 eq) were added then heated to reflux temperature for 45 min. The progress of the reaction was monitored by TLC. The TLC shows complete consumption of starting material. After which was concentrated under reduced pressure. The residue was purified by column chromatography on silica gel to afford 2-chloro-5-((2-chloro-4-(fluoromethyl) thiophen-3-ylamino)methyl)-N-methylpyrimidin-4-amine (54) (0.25 g, 51.02%) as a pale yellow solid. LCMS: m/z=321.01 [M+H]⁺, 94.13% (3.38 min).

### Step-3: 7-chloro-3-(2-chloro-4-(fluoromethyl) thiophen-3-yl)-1-methyl-3,4-dihydropyrimido[4,5-d]pyrimidin-2(1H)-one (55)

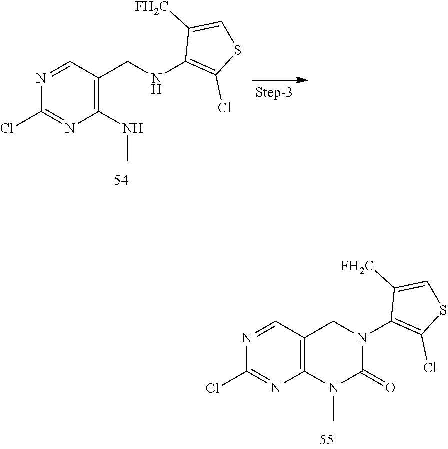

To a solution of 2-chloro-5-((2-chloro-4-(fluoromethyl)thiophen-3-ylamino)methyl)-N-methylpyrimidin-4-amine (54) (0.25 g, 0.781 mmol 1.0 eq.) in Dry THF (2.0 mL), triphosgene (0.25 g, 0.858 mmol 1.1 eq.) in THF (1 mL) were added followed by Et₃N (0.94 g, 0.937 mmol 1.2 eq.) added, then stirred at Room temperature for 2 h, To the reaction mixture K₂CO₃ (1.1 g, 7.810 mmol 10.0 eq.) and ACN (1 mL) were added, then stirred at 55° C. for 16 h. The progress of the reaction was monitored by TLC. The TLC shows complete consumption of starting material. The reaction mixture was filtered and the filtrate was concentrated to get crude compound. The residue was purified by column chromatography on silica gel to afford 7-chloro-3-(2-chloro-4-(fluoromethyl)thiophen-3-yl)-1-methyl-3,4-dihydropyrimido[4,5-d]pyrimidin-2(1H)-one (55) (0.12 g, 47.16%) as a gummy liquid. LCMS: m/z=346.98 [M+H]+, 96.56% (3.23 min).

### Step-4: 3-(2-chloro-4-(fluoromethyl) thiophen-3-yl)-7-(2-methoxy-4-(1-methylpiperidin-4-yl) phenylamino)-1-methyl-3,4-dihydropyrimido[4,5-d]pyrimidin-2(1H)-one (AA11)

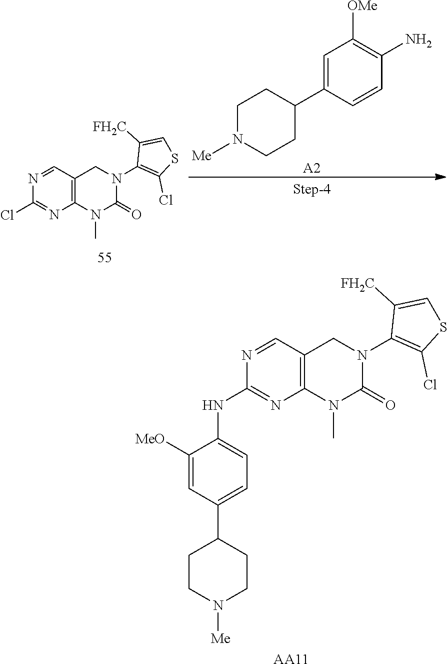

To a solution of 7-chloro-3-(2-chloro-4-(fluoromethyl)thiophen-3-yl)-1-methyl-3,4-dihydropyrimido[4,5-d]pyrimidin-2(1H)-one (55) (0.12 g, 0.345 mmol 1.0 eq.) and 2-methoxy-4-(1-methylpiperidin-4-yl)aniline (A2) (0.091 g, 0.414 mmol 1.2 eq) in n-BuOH (2 mL) was added Trifluroacetic acid (0.08 g, 0.690 mmol, 2.0 eq.) and stirred at 100° C. for 6 h. The progress of the reaction was monitored by TLC. The TLC shows complete consumption of starting material The reaction mixture was concentrated and was purified with Combiflash Rf with Teledyne IscoRediSepRfHigh Performance Gold or SilicycleSiliaSep High Performance columns (40, 80, or 120 g) to afford 3-(2-chloro-4-(fluoromethyl)thiophen-3-yl)-7-(2-methoxy-4-(1-methylpiperidin-4-yl)phenylamino)-1-methyl-3,4-dihydropyrimido[4,5-d]pyrimidin-2(1H)-one (AA11) (30 mg, 16.4%) as a white solid. Analytical data for this synthesised compound AA11 is shown in Table 1.1.

### Compound AA12

### Synthesis of 3-(2-chloro-4-(fluoromethyl) thiophen-3-yl)-7-(2-methoxy-4-(1-methylpiperidin-4-yl) phenylamino)-1-(2-methoxyethyl)-3,4-dihydropyrimido[4,5-d]pyrimidin-2(1H)-one (AA12)

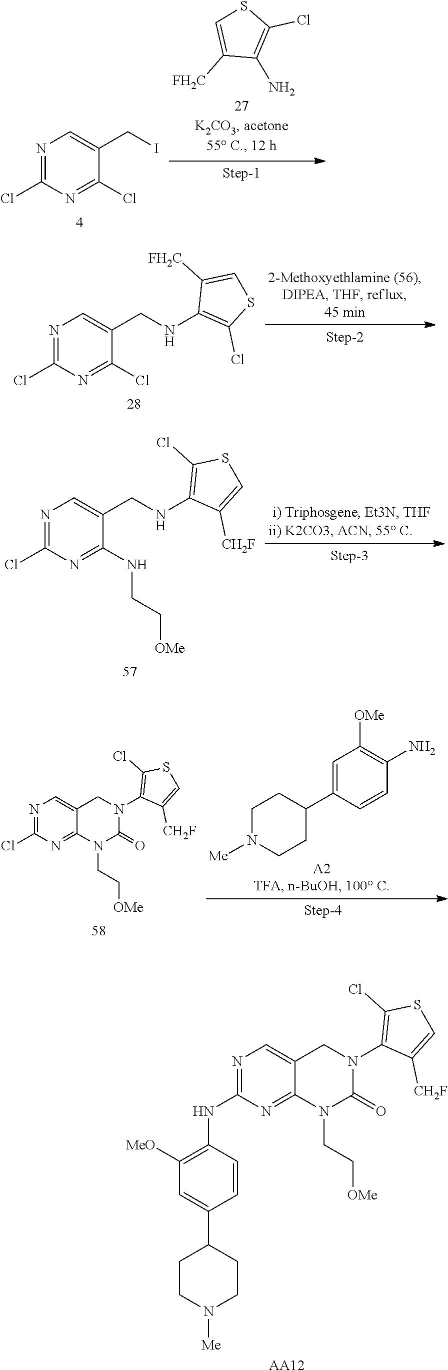

### Step-1: Preparation of Compound 28

The procedure for the preparation of compound 28 was described in step-1 for the synthesis of compound AA3.

### Step-2: 2-chloro-5-((2-chloro-4-(fluoromethyl) thiophen-3-ylamino) methyl)-N-(2-methoxyethyl) pyrimidin-4-amine (57)

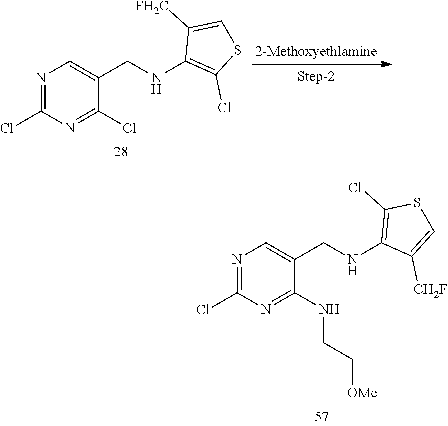

A mixture of 2-chloro-N-((2,4-dichloropyrimidin-5-yl)methyl)-4-(fluoromethyl)thiophen-3-amine (28) (0.2 g, 0.613 mmol 1.0 eq.), 2-Methoxyethylamine (56) (0.23, 3.065, 5.0 eq) in Dry THF (6 mL) DIPEA (0.99 g, 12.30 mmol, 8.0 eq) were added then heated to reflux temperature for 45 mins. The progress of the reaction was monitored by TLC. The TLC shows complete consumption of starting material. After which was concentrated under reduced pressure. The residue was purified by column chromatography on silica gel to afford 2-chloro-5-((2-chloro-4-(fluoromethyl) thiophen-3-ylamino) methyl)-N-(2-methoxyethyl) pyrimidin-4-amine (57) (0.2 g, 90.90%) as a pale yellow solid. LCMS: m/z=365.04 [M+H]⁺, 77.08% (3.53 min).

### Step-3: 7-chloro-3-(2-chloro-4-(fluoromethyl) thiophen-3-yl)-1-(2-methoxyethyl)-3,4-dihydropyrimido[4,5-d]pyrimidin-2(1H)-one (58)

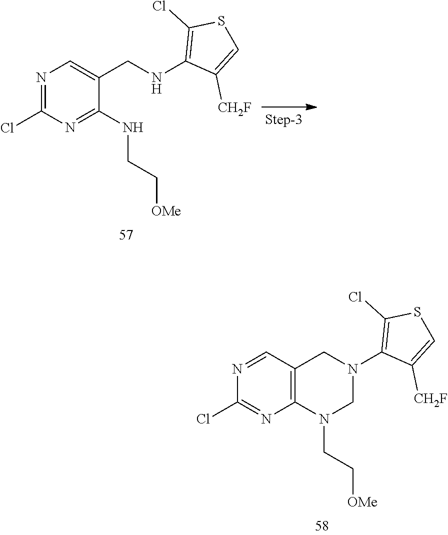

To a solution of 2-chloro-5-((2-chloro-4-(fluoromethyl) thiophen-3-ylamino) methyl)-N-(2-methoxyethyl) pyrimidin-4-amine (57) (0.2 g, 0.549 mmol 1.0 eq.) in Dry THF (2.0 mL) triphosgene (0.18 g, 0.603 mmol 1.1 eq.) in THF (1 mL) were added followed by Et₃N (0.066 g, 0.658 mmol 1.2 eq.) added, then stirred at Room temperature for 2 h, To the reaction mixture K₂CO₃ (1.1 g, 7.810 mmol 10.0 eq.) and ACN (1 mL) were added, then stirred at 55° C. for 16 h. The progress of the reaction was monitored by TLC. The TLC shows complete consumption of starting material. The reaction mixture was filtered and the filtrate was concentrated to get crude compound. The residue was purified by column chromatography on silica gel to afford 7-chloro-3-(2-chloro-4-(fluoromethyl) thiophen-3-yl)-1-(2-methoxyethyl)-3, 4-dihydropyrimido [4,5-d]pyrimidin-2(1H)-one (58) (0.1 g, 47.61%) as a gummy liquid. LCMS: m/z=391.05 [M+H]⁺, 89.32% (3.22 min).

### Step-4: 3-(2-chloro-4-(fluoromethyl) thiophen-3-yl)-7-(2-methoxy-4-(1-methylpiperidin-4-yl)phenylamino)-1-(2-methoxyethyl)-3,4-dihydropyrimido[4,5-d]pyrimidin-2(1H)-one (AA12)

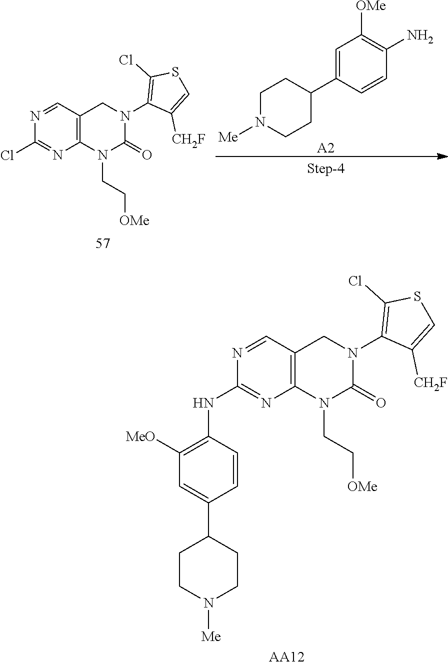

To a solution of 7-chloro-3-(2-chloro-4-(fluoromethyl) thiophen-3-yl)-1-(2-methoxyethyl)-3, 4-dihydropyrimido [4,5-d]pyrimidin-2(1H)-one (57) (0.1 g, 0.256 mmol 1.0 eq.) and 2-methoxy-4-(1-methylpiperidin-4-yl)aniline (A2) (0.067 g, 0.307 mmol 1.2 eq) in n-BuOH (2 mL) was added Trifluroacetic acid (0.06 g, 0.512 mmol, 2.0 eq.) and stirred at 100° C. for 6 h. The progress of the reaction was monitored by TLC. The TLC shows complete consumption of starting material The reaction mixture was concentrated and was purified with Combiflash Rf with Teledyne IscoRediSepRfHigh Performance Gold or SilicycleSiliaSep High Performance columns (40, 80, or 120 g) to afford 3-(2-chloro-4-(fluoromethyl)thiophen-3-yl)-7-(2-methoxy-4-(1-methylpiperidin-4-yl)phenylamino)-1-(2-methoxyethyl)-3,4-dihydropyrimido[4,5-d]pyrimidin-2(1H)-one (AA12) (40 mg, 27.3%) as a white solid. Analytical data for this synthesised compound AA12 is shown in Table 1.1.

### Compound AA13

### Synthesis of 3-(2-chloro-4-(fluoromethyl) thiophen-3-yl)-7-(4-(1,2-dimethylpiperidin-4-yl)-2-methoxyphenylamino)-1-(5-methoxypyridin-2-yl)-3,4-dihydropyrimido[4,5-d]pyrimidin-2(1H)-one (AA13)

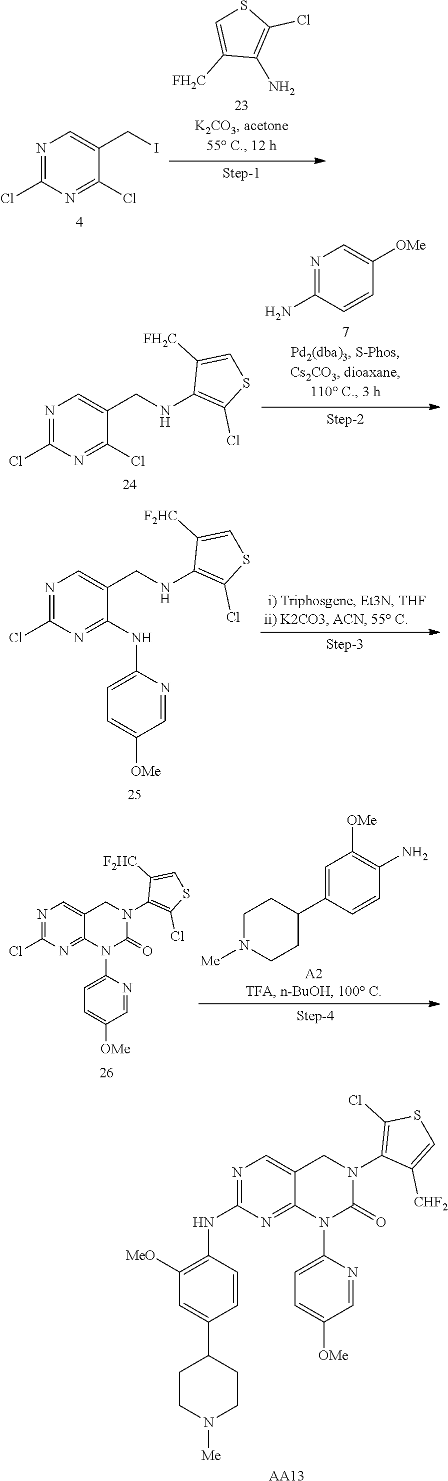

### Step-1: Preparation of Compound 24

The procedure for the preparation of compound 24 was described in step-1 for the synthesis of compound AA2.

### Step-2: Preparation of Compound 25

The procedure for the preparation of compound 25 was described in step-2 for the synthesis of compound AA2.

### Step-3: Preparation of Compound 26

The procedure for the preparation of compound 26 was described in step-3 for the synthesis of compound AA2.

### Step-4: 3-(2-chloro-4-(fluoromethyl) thiophen-3-yl)-7-(4-(1,2-dimethylpiperidin-4-yl)-2-methoxyphenylamino)-1-(5-methoxypyridin-2-yl)-3,4-dihydropyrimido[4,5-d]pyrimidin-2(1H)-one (AA13)

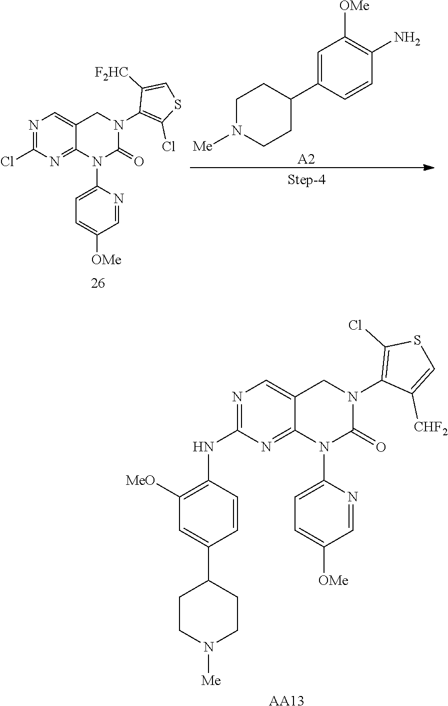

To a solution of 7-chloro-3-(2-chloro-4-(fluoromethyl)thiophen-3-yl)-1-(5-methoxypyridin-2-yl)-3,4-dihydropyrimido[4,5-d]pyrimidin-2(1H)-one (26) (0.13 g, 0.284 mmol 1.0 eq.) and 2-methoxy-4-(1-methylpiperidin-4-yl)aniline (A2) (0.093 g, 0.426 mmol 1.5 eq) in n-BuOH (2 mL) was added Trifluroacetic acid (0.066 g, 0.568 mmol, 2.0 eq.) and stirred at 100° C. for 6 h. The progress of the reaction was monitored by TLC. The TLC shows complete consumption of starting material The reaction mixture was concentrated and was purified with Combiflash Rf with Teledyne IscoRediSepRfHigh Performance Gold or SilicycleSiliaSep High Performance columns (40, 80, or 120 g) to afford 3-(2-chloro-4-(fluoromethyl)thiophen-3-yl)-7-(4-(1,2-dimethylpiperidin-4-yl)-2-methoxyphenylamino)-1-(5-methoxypyridin-2-yl)-3,4-dihydropyrimido[4,5-d]pyrimidin-2(1H)-one (AA13) (45 mg, 24.8%) as a white solid. Analytical data for this synthesised compound AA12 is shown in Table 1.1.

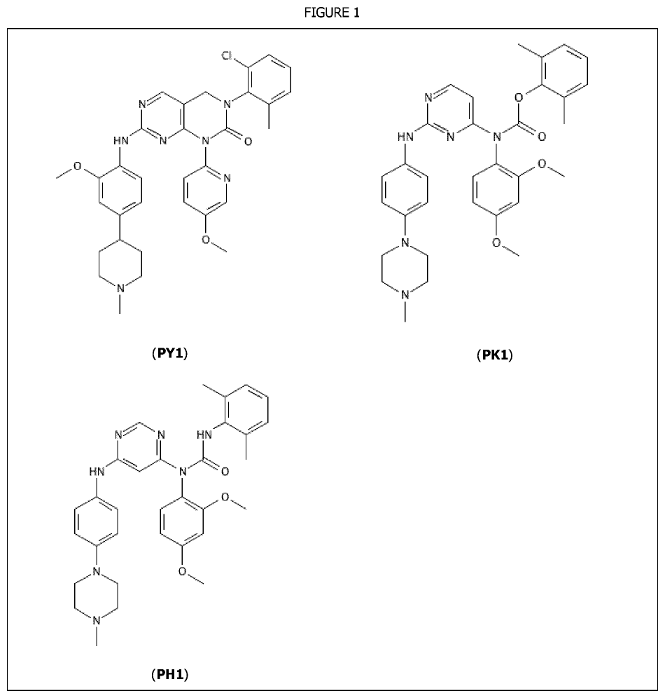

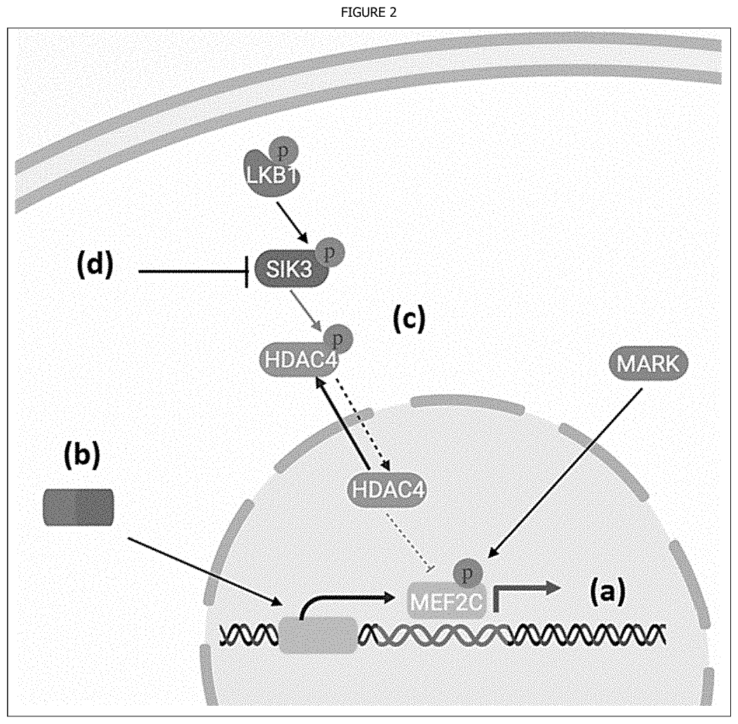

The structures of compounds PY1 and AA1 to AA13 are shown in FIG. 1 and FIG. 3 A, and the analytical data from their synthesis are shown in Table 1.1.

TABLE 1.1
Synthesis of kinase inhibitors.

| Com- | Mol. Wt. | |
|---|---|---|
| pound | LCMS: | |
| Number | m/z [M + H]⁺ | ¹H NMR |
| PY1 | 600.39 | ¹H NMR (400 MHz, DMSO-d6): δ 8.31 |
| | 99.4% | (d, J = 2.8 Hz, 1H), 8.21 (s, |
| | (9.06 min) | 1H), 7.73 (s, 1H), 7.58 (dd, J = 8.8, |
| | | 3.2 Hz, 1H), 7.55 (dd, J = 2.8, |
| | | 2.0 Hz, 1H), 7.41 (d, J = 8.8 Hz, 1H), |
| | | 7.36-7.34 (m, 2H), 7.25 (d, J = |
| | | 8.0 Hz, 1H), 6.80 (d, J = 1.6 Hz , 1H), 6.37 (d, J = 8.0 Hz, 1H ), |
| | | 4.74-4.65 (m, 2H ), 3.93 (s, 3H), 3.79 (s, 3H), 2.86 (br d, J = 11.6 |
| | | Hz, 1H), 2.32 (s, 4H), 2.19 (s, 3H), 1.97-1.90 (m, 2H), 1.70-1.56 |
| | | (m, 5H). |
| AA1 | 606.19 | ¹H NMR (400 MHz, DMSO-d6): δ |
| | 99.06% | 8.314 (d, J = 2.8 Hz, 1H), 8.22 |
| | (10.01 min) | (s, 1H), 7.73 (s, 1H), 7.60 (dd, J = 8.4, |
| | | 3.2 Hz, 1H), 7.42 (d, J = |
| | | 8.4 Hz, 1H), 7.25-7.22 (m, 2H), 6.80 |
| | | (d, J = 1.6 Hz, 1H), 6.37 (d, |
| | | J = 8.0 Hz, 1H ), 4.72 (q, J = 14.8, |
| | | 2.4 Hz, 2H ), 3.93 (s, 3H), |
| | | 3.79 (s, 3H), 2.85 (d, J = 11.2 Hz, 2H), 2.19 (s, 3H), 2.11 (d, J = |
| | | 1.2 Hz, 3H), 1.98-1.92 (m, 2H), 1.70-1.56 (m, 5H). |
| AA2 | 612.37 | ¹H NMR (400 MHz, DMSO-d6): δ 9.43 |
| | 99.26% | (s, 1H), 8.33 (d, J = 2.8 Hz, |
| | (6.46 min) | 1H), 8.25 (s, 1H), 7.96 (s, 1H), |
| | | 7.63 (dd, J = 8.4, 3.2 Hz, 1H), |
| | | 7.42 (d, J = 8.8 Hz, 1H), 7.14-7.09 |
| | | (m, 2H), 6.85-6.82 (m, 2H), |
| | | 4.80 (d, J = 14 Hz, 1H ), 4.67 (d, J = 41.4 Hz , 1H ), 3.94 (s, 3H), |
| | | 2.86 (d, J = 11.2 Hz, 2H), 2.32-2.30 (m, 1H), 2.20 (s, 3H), 1.98- |
| | | 1.94 (m, 2H), 1.67-1.54 (m, 5H). |
| AA3 | 594.35 | ¹H NMR (400 MHz, DMSO-d6): δ |
| | 95.20% | 9.42 (s, 1H), 8.32 (d, J = 2.8 Hz, |
| | (3.62 min) | 1H), 8.20 (s, 1H), 7.75 (d, J = 4 Hz, |
| | | 1H), 7.62 (dd, J = 11.6 Hz, |
| | | 1H), 7.44 (d, J = 8.8 Hz, 1H), 7.12 |
| | | (d, J = 8.0 Hz, 2H), 6.84 (d, J = |
| | | 8.0 Hz, 2H), 5.30 (d, J = 47.6 Hz, 2H), 4.73 (q, J = 24.8, 14.0 |
| | | Hz, 2H ), 3.94 (s, 3H), 2.86 (d, J = 10.8 Hz, 2H), 2.33-2.30 (m, |
| | | 1H), 2.20 (s, 3H) , 1.98-1.93 (m, 2H), 1.67-1.53 (m, 4H). |
| AA4 | 584.31 | ¹H NMR (400 MHz, DMSO-d6): δ |
| | 98.7% | 8.30 (d, J = 3.2 Hz, 1H), 8.17 (s, |
| | (6.90 min) | 1H), 7.65 (s, 1H), 7.60 (dd, J = 8.4, |
| | | 3.2 Hz, 1H), 7.41 (d, J = 8.8 |
| | | Hz, 1H), 7.27 (br d, J = 8.0 Hz, 1H), |
| | | 6.79 (s, 1H), 6.36 (d, J = 8.8 |
| | | Hz, 1H ), 4.64 (d, J = 5.6 Hz, 1H ), 3.93 (s, 3H), 3.79 (s, 3H), |
| | | 3.65 (s, 3H), 2.86 (d, J = 10.8 Hz, 2H), 2.32-2.34-2.30 (m, 1H), |
| | | 2.19 (s, 3H), 2.13 (s, 3H), 2.04 (s, 3H), 1.96-1.90 (m, 2H), 1.69- |
| | | 1.58 (m, 5H). |
| AA5 | 571.25 | ¹H NMR (400 MHz, DMSO-d6): δ 8.31 |
| | 98.8% | (d, J = 3.2 Hz, 1H), 8.18 (s, |
| | (7.72 min) | 1H), 7.70 (s, 1H), 7.59 (dd, J = 8.8, |
| | | 2.8 Hz, 1H), 7.42 (d, J = 8.8 |
| | | Hz, 1H), 7.25 (m, J = 8.8 Hz, 1H), 6.80 (s, 1H), 6.36 (d, J = 8.8 |
| | | Hz, 1H ), 4.76 (s, 2H ), 3.93 (s, 3H), 3.79 (s, 3H), 2.85 (br d, J = |
| | | 8.8 Hz, 2H), 2.38 (s, 3H), 2.19 (s, 3H), 2.17 (s, 3H), 1.96-1.91 |
| | | (m, 2H), 1.69-1.58 (m, 5H). |
| AA6 | 595.19 | ¹H NMR (400 MHz, DMSO-d6): δ |
| | 95.80% | 9.25 (s, 1H), 8.31 (d, J = 2.8 Hz, |
| | (3.14 min) | 1H), 8.20 (s, 1H), 7.75 (d, J = 2.8 Hz, |
| | | 1H), 7.62 (dd, J = 8.8, 3.2 |
| | | Hz, 1H), 7.42 (d, J = 8.8 Hz, 1H), |
| | | 7.07-7.05 (m, 2H), 6.56 (d, J = |
| | | 8.8 Hz, 2H), 5.30 (d, J = 47.6 Hz, 2H ), 4.70 (q, J = 14.4 Hz, 2H), |
| | | 3.93 (s, 3H), 2.99-2.97 (m, 4H), 2.45-2.42 (m, 4H), 2.2 (s, 3H). |
| AA7 | 624.32 | ¹H NMR (400 MHz, DMSO-d6): δ |
| | 97.89% | 8.32 (d, J = 2.8 Hz, 1H), 8.23 (s, |
| | (3.77 min) | 1H), 7.75 (d, J = 3.2 Hz, 2H), 7.61 |
| | | (dd, J = 8.8, 2.8 Hz, 1H), 7.44 |
| | | (d, J = 8.8 Hz, 1H), 7.24 (br d, J = 8.0 Hz, 1H), 6.81 (d, J = 8.8 |
| | | Hz, 1H), 6.38 (d, J = 8.8 Hz, 1H), 5.30 (d, J = 47.6 Hz, 2H), 4.73 |
| | | (q, J = 14.4 Hz, 2H ), 3.93 (s, 3H), 3.79 (s, 3H), 2.85 (br d, J = |
| | | 8.8 Hz, 2H), 2.45-2.42 (m, 1H), 2.19 (s, 3H) , 1.97-1.92 (m, 2H), |
| | | 1.70-1.58 (m, 4H). |
| AA8 | 622.32 | ¹H NMR (400 MHz, DMSO-d6): δ |
| | 98.32% | 9.42 (s, 1H), 8.32 (d, J = 2.8 Hz, |
| | (10.86 min) | 1H), 8.24 (s, 1H), 7.74 (d, J = 4.0 Hz, |
| | | 1H), 7.61 (dd, J = 8.8, 2.8 |
| | | Hz, 1H), 7.45 (d, J = 8.8 Hz, 1H), 7.12 (d, J = 8.4 Hz, 2H), 6.83- |
| | | 6.80 (m, 2H), 5.36 (d, J = 47.6 Hz, 2H), 4.73 (q, J = 14.4 Hz, |
| | | 2H), 3.95 (s, 3H), 2.22 (br s, 1H), 2.18 (s, 3H), 2.13- 2.09 (m, |
| | | 2H), 1.65-1.61 (m, 2H), 1.31-1.28 (d, J = 12.0 Hz 2H), 1.08 (s, |
| | | 3H), 1.06 (s, 3H). |
| AA9 | 608.62 | ¹H NMR (400 MHz, DMSO-d6): δ |
| | 96.66% | 9.42 (s, 1H), 8.32 (d, J = 2.8 Hz, |
| | (10.59 min) | 1H), 8.24 (s, 1H), 7.74 (d, J = 4.0 Hz, |
| | | 1H), 7.61 (dd, J = 8.8, 2.8 |
| | | Hz, 1H), 7.44 (d, J = 8.8 Hz, 1H), 7.13 (d, J = 8.4 Hz, 2H), 6.82 |
| | | (d, J = 8.8 Hz, 2H), 5.36 (d, J = 47.6 Hz, 2H), 4.71 (q, J = 14.4 |
| | | Hz, 2H), 3.94 (s, 3H), 2.89-2.83 (m, 1H), 2.18 (s, 3H), 2.09-2.03 |
| | | (m 1H), 1.94-1.89 (m, 1H), 1.65-1.61 (m, 2H), 1.56-1.51 (m 1H), |
| | | 1.29-1.20 (m 2H), 1.04 (d, J = 6.4 Hz, 3H). |
| AA10 | 623.28 | ¹H NMR (400 MHz, DMSO-d6): δ |
| | 97.8% | 8.21 (s, 1H), 7.74 (d, J = 3.6 Hz, |
| | (11.74 min) | 1H), 7.70 (s, 1H), 7.41 (d, J = 8.8 Hz, |
| | | 1H), 7.26-7.23 (m, 2H), |
| | | 7.08-7.05 (m, 2H), 6.80 (d, J = 1.6 Hz, 1H), 6.36 (d, J = 7.6 Hz, |
| | | 1H), 5.30 (d, J = 47.6 Hz, 2H), 4.68 (q, J = 14.4 Hz, 2H), 4.03 |
| | | (m, 1H), 3.86 (s, 3H), 3.80 (s, 3H), 3.57-3.55 (m, 1H), 3.50 (s, |
| | | 3H), 3.16-3.12 (m, 2H), 1.82-1.66 (m, 5H). |
| AA11 | 531.29 | ¹H NMR (400 MHz, DMSO-d6): δ |
| | 99.47% | 8.11 (s, 1H), 8.00 (d, J = 8.0 Hz, |
| | (4.26 min) | 1H), 7.96 (s, 1H), 7.73 (d, J = 4 Hz, |
| | | 1H), 6.91 (d, J = 1.6 Hz, 1H, |
| | | 1H), 6.81 (dd, J = 8.0, 2.0 Hz, 1H), 5.32 (q, J = 8.4 Hz, 1H), 5.18 |
| | | (q, J = 8.4 Hz, 1H), 4.58 (q, J = 14.4 Hz, 2H), 3.85 (s, 3H), 3.29 |
| | | (s, 3H), 2.89 (br d, J = 10.4 Hz, 2H), 2.22 (s, 3H), 2.08-1.93 (m, |
| | | 2H), 1.76-1.66 (m, 5H). |
| AA12 | 575.29 | ¹H NMR (400 MHz, DMSO-d6): δ |
| | 98.45% | 8.12 (s, 1H), 8.03 (s, 1H), 7.91 |
| | (4.20 min) | (d, J = 8.0 Hz, 1H ), 7.73 (d, J = 4.0 |
| | | Hz, 1H), 6.92 (d, J = 1.6 |
| | | Hz, 1H), 6.78 (dd, J = 8.0, 1.6 Hz, 1H), 5.28 (dq, J2 = 11.2 Hz, |
| | | J₂₋₃ = 16.4 Hz, 2H), 4.50 (q, J = 14.4 Hz, 2H ), 4.13 (t, J = 6.0 |
| | | Hz, 2H), 3.85 (s, 3H), 3.52 (t, J = 6.0 Hz, 2H), 3.23 (s, 3H), 2.89 |
| | | (br d, J = 10.4 Hz, 2H), 2.21 (s, 3H), 1.99 (m, 2H), 1.76-1.66 (m, |
| | | 5H). |
| AA13 | 642.30 | ¹H NMR (400 MHz, DMSO-d6): δ |
| | 97.34% | 8.31 (d, J = 2.8 Hz, 1H), 8.23 (s, |
| | (3.84 min) | 1H), 7.97 (d, J = 1.6 Hz, 1H), |
| | | 7.75 (m, 1H), 7.61 (dd, J = 8.8, 3.2 |
| | | Hz, 1H), 7.40 (d, J = 8.8 Hz, 1H), 7.23 (d, J = 8.4 Hz, 1H), 7.08- |
| | | 6.80 (m, 2H), 6.37 (d, J = 8.8 Hz, 1H), 4.73 (q, J = 54.2 Hz, 2H ), |
| | | 3.93 (s, 3H), 3.79 (s, 3H), 2.87 (d, J = 11.2 Hz, 2H), 2.19 (s, 3H), |
| | | 1.96-1.91 (m, 2H), 1.70-1.56 (m, 5H). |

### Example 1.2 [Prophetic]: Synthesis of Further Compounds of Formula (I), Including Those Macrocyclic Compounds of Formulae (VIII)

Further compounds of Formula (I) are synthesised based on the structure and activity of related structures disclosed in WO2015/006492, in particular compounds are made that are based formula I thereof, yet with a moiety Q thereof analogous to any R⁶ disclosed herein, or in co-pending PCT/EP2019/078751 (to the present applicant).

For example, compounds of Formula (I) are made as analogues to formula IVA of WO2015/006492, in each case with a moiety Q thereof analogous to any R⁶ disclosed herein, or in co-pending PCT/EP2019/078751.

Furthermore, macrocyclic compounds of Formula (VIII)) are synthesised based on the structure and activity of related structured disclosed in WO2016/023014, in particular compounds are made that are based formula I thereof, yet with a ring C thereof analogous to any R⁶ disclosed herein, or in co-pending PCT/EP2019/078751.

In particular, the cyclisation/metathesis procedure to synthesise macrocyclic compounds of Formula (VIII) can be, after the teaching of the present invention, readily derived by the person of ordinary skill from the equivalent cyclisation/metathesis procedure described in WO2016/023104. For example, starting from the intermediate product of step 4 of the general schemes 1 to 3 above, compounds of Formula (VIII) are synthesised according to general scheme 4 (where such scheme, although shown with an E double bond within the macrocyclic linker L′, is also applicable for the synthesis of corresponding compounds with a Z double bond):

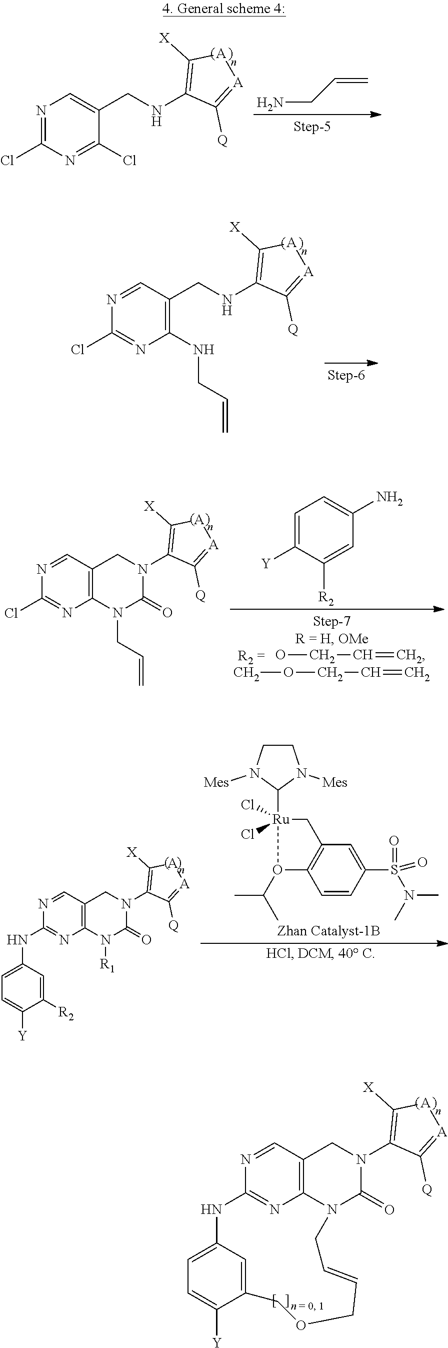

The preparation of the two allylamines for cyclisation can be readily derived by the person of ordinary skill from the equivalent procedures described in WO2017/011590 and WO2008/140419 (for example, as depicted respectively below).

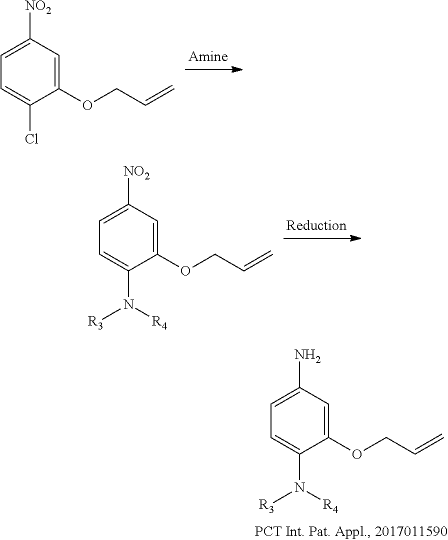

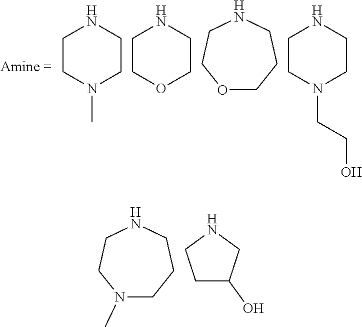

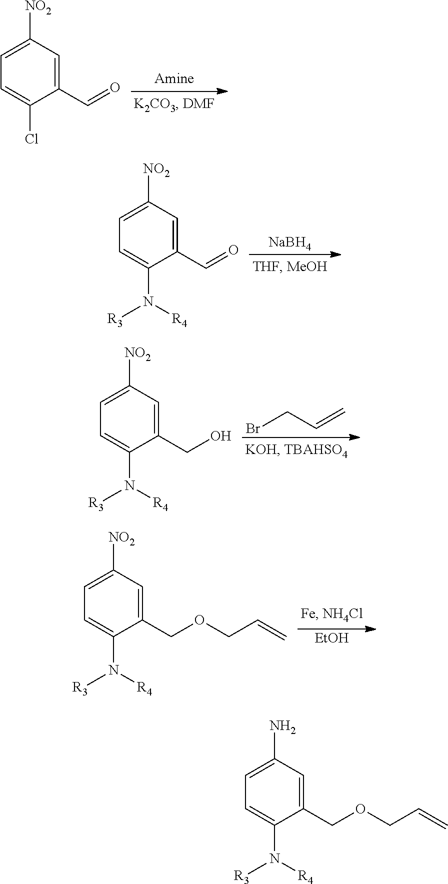

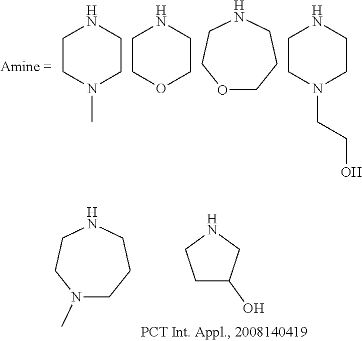

For Y=N-methyl-piperazine (attachment of R^{1a} to Q via carbon):

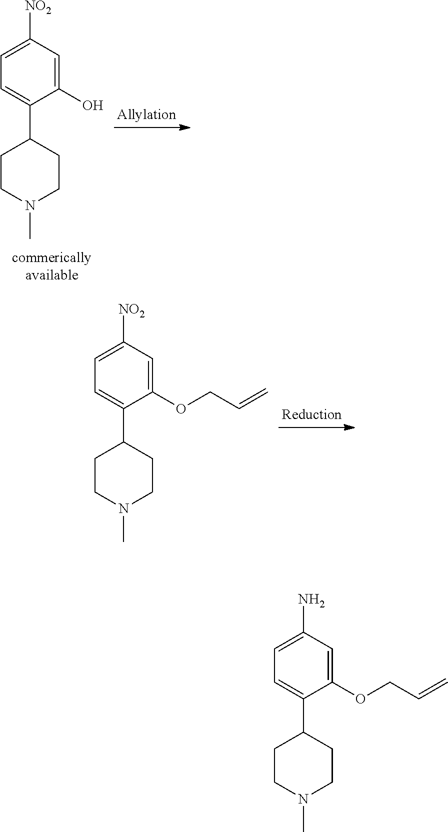 [SMILES: CN1CCC(c2ccc([N+](=O)[O-])cc2O)CC1 ; C=CCOc1cc([N+](=O)[O-])ccc1C1CCN(C)CC1 ; C=CCOc1cc(N)ccc1C1CCN(C)CC1]

Alternatively:

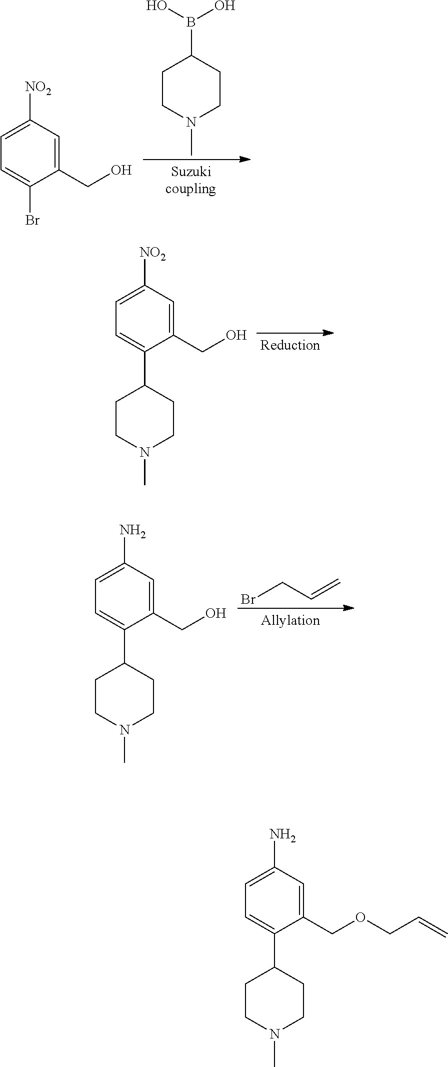

And more generally:

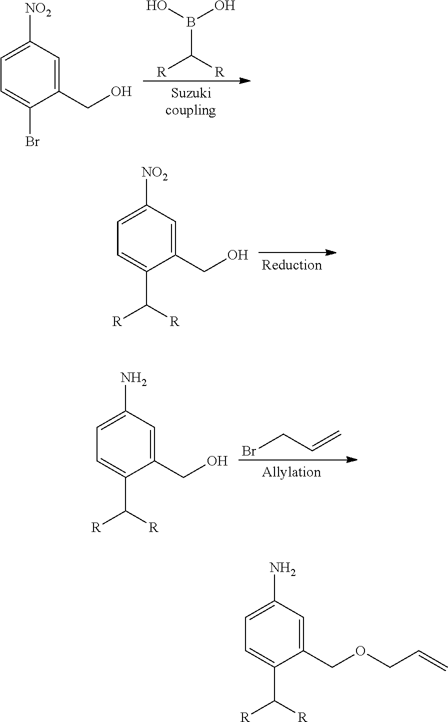

Specific examples of compounds of Formula (VIII) are synthesised, with various indicative 5- and 6-membered linkers containing a C═C double bond in either Z- or C-configuration, including those with the indicative X and Y moieties shown below.

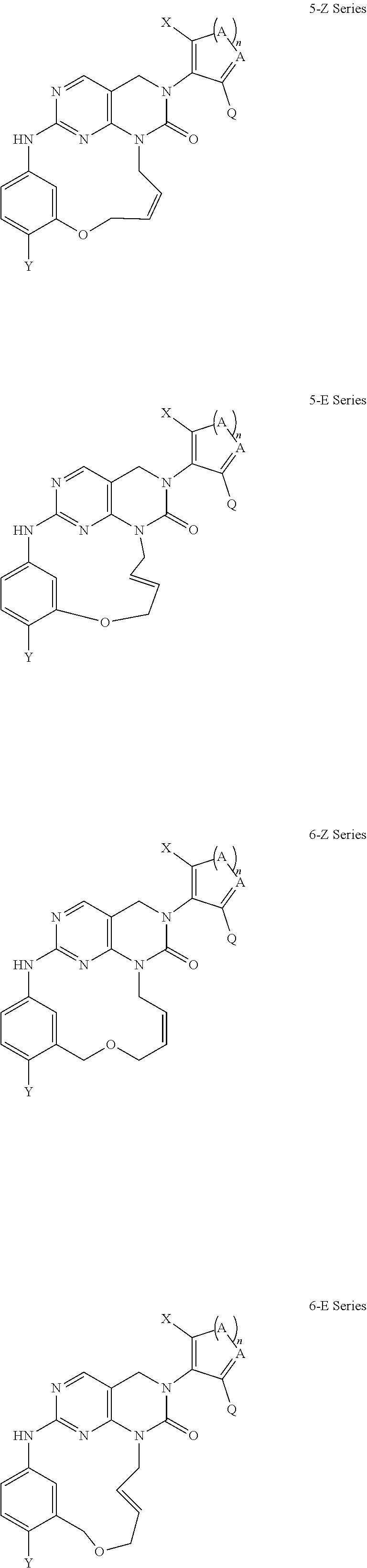

X and Q=R⁷; A=C, N, O, S with at least one A being N, O, S. n>0;

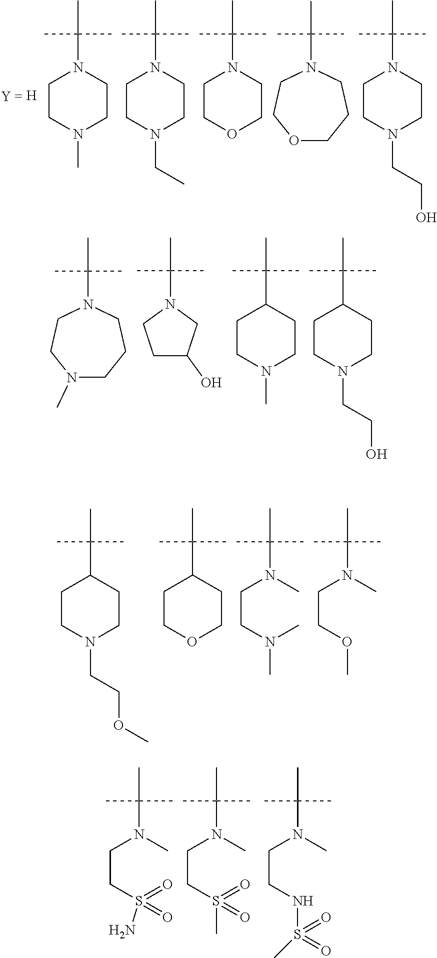

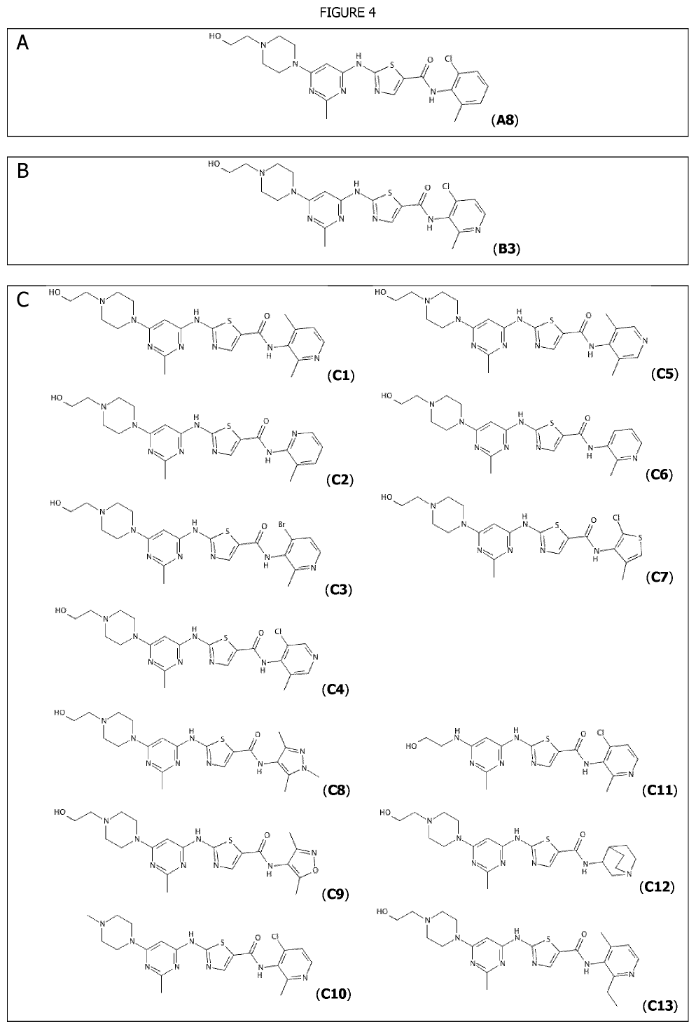

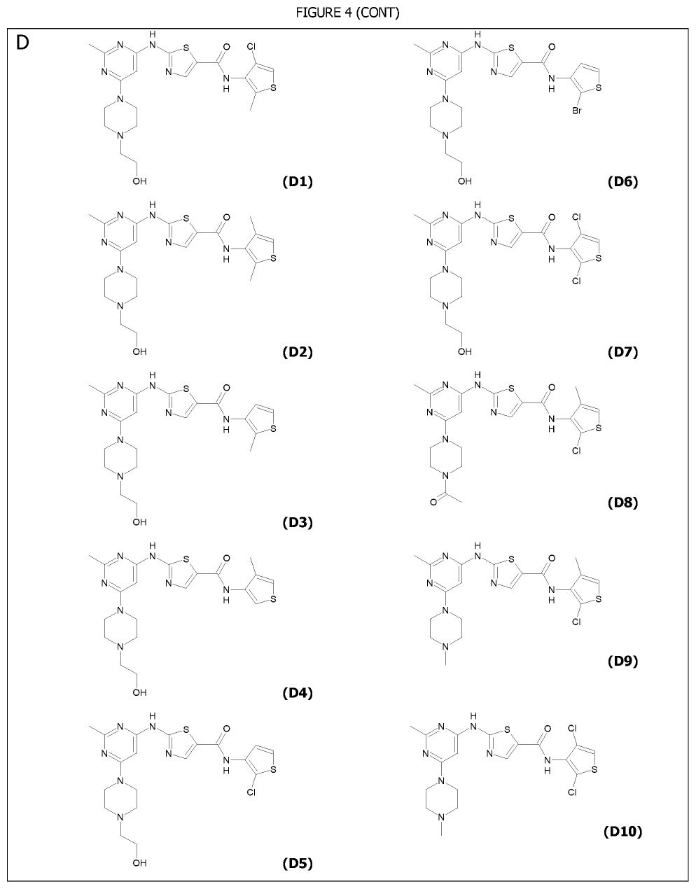

Specific examples of compounds of Formula (VIII) are synthesised, with various indicative R5 (-L-R6) moieties as shown by: (a) the amide-linked heterocyclic and heteroaromatic moieties in compounds B3, C1 to C13 and D1 to D10 (FIGS. 4 B, C and D); (b) those R5 (-L-R6) moieties as shown in compounds AA1 to AA224 (FIG. 3 A); and (c) the moieties shown below.

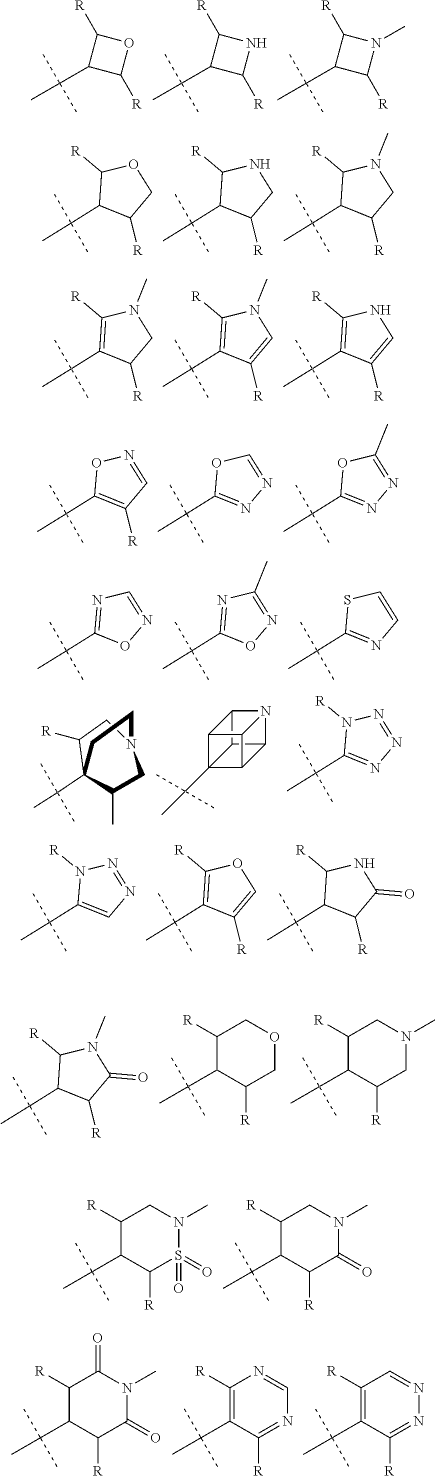

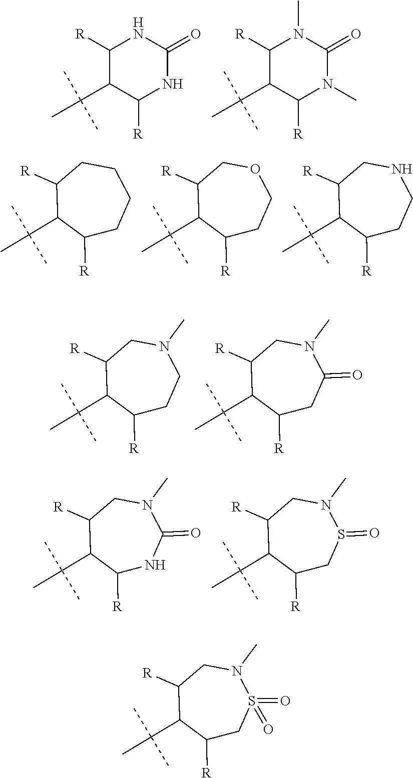

The synthesis teachings of WO2015/006492 and WO2016/023014, as applicable, together with those herein and in PCT/EP2019/078751 with respect to R⁶ moieties, are used to design and effect the synthesis of such further compounds of Formula (I), and the synthesis of the applicable reactants is described by WO2008/140419 and WO2017/011590 (the synthesis teachings of such patent applications, in particular Example 1 of PCT/EP2019/078751, hereby incorporated herein by reference).

In particular, the biological activities of the related structures disclosed in WO2015/006492 from Example 93 thereof (and in particular, that shown in Tables A to D, and hereby incorporated herein by reference) are used to guide SAR of compounds of Formulae (1); and in particular, biological activities related to SIK2 and SIK3 described in Sundberg et al (2016) and WO 216/023014 (and hereby incorporated herein by reference) are also used to guide SAR of compounds of Formulae (I), and of compounds of Formula (VIII).

### Example 2.1: Inhibition of SIK2 and SIK3 by Compounds of Formula (I)

The inventors demonstrated that, surprisingly, the compounds of Formula (I) disclosed herein, despite the significant structural changes compared to the prior art compound PY1, potently inhibit SIK kinases, with many compounds inhibiting SIK3 and/or SIK2 with low double-digit nM IC50s (Table 2.1.1). In particular, the inventors were surprised to observe that the kinases inhibitors AA2, AA3, AA6, AA8, AA9 and AA12 were superior in inhibiting SIK2 and SIK3 compared to PY1 (prior art compound YKL-05-099), and very surprisingly, compounds of Formula (I) (eg AA11, AA12 and AA13) even showed increased selectively towards SIK3 over SIK2.

TABLE 2.1.1
Biological activity of compounds of Formula (I)

| | Kinase: IC50 (nM) | |
|---|---|---|
| Compound | SIK2 | SIK3 |
| PY1 | 50-100 | 100-200 |
| (YKL-05-099) | | |
| AA1 | 100-200 | 200-400 |
| AA2 | <50 | <50 |
| AA3 | <50 | <50 |
| AA4 | 5,000-10,000 | >10,000 |
| AA5 | 400-800 | 800-1,000 |
| AA6 | <50 | <50 |
| AA7 | 50-100 | 100-200 |
| AA8 | <50 | <50 |
| AA9 | <50 | <50 |
| AA10 | 50-100 | 100-200 |
| AA11 | 100-200 | <50 |
| AA12 | <50 | <50 |
| AA13 | 100-200 | 100-200 |

Other compounds of Formula (I) (such as those of Formulae (VIII)), for example those shown in FIGS. 3 A and B and/or are synthesised in Example 1.2, are similarly tested for their inhibitory activity against SIK2 and SIK3 kinases, in particular to investigate their potency against and/or selectively for SIK3.

The IC50s of the compounds against the SIK family of protein-serine/threonine kinases (SIK2 and SIK3) were determined as followed. Briefly, a radiometric protein kinase assay (33PanQinase® Activity Assay) was used for measuring the kinase activity of the two protein kinases. The kinase assays were performed in 96-well FlashPlates™ from PerkinElmer (Boston, Mass., USA) in a 50 uL reaction volume. The reaction cocktail was pipetted in four steps in the following order:
25 uL of assay buffer (standard buffer/[gamma-33P]-ATP)
10 uL of ATP solution (in water)
5 uL of test compound (in 10% DMSO)
20 uL enzyme/substrate mix

The assay contained 70 mM HEPES-NaOH pH7.5, 3 mM MgCl2, 3 mM MnCl₂, 3 μM Na-orthovanadate, 1.2 mM DTT, ATP (variable concentrations, corresponding to the apparent ATP-Km of the respective kinase, see Table 2B), [gamma-33P]-ATP (approx. 8×10⁵ cpm per well), protein kinase (variable amount, see Table 2B), and substrate (variable amounts, see Table 2.1.2).

The following amounts of enzyme and applicable substrate were used per well:

TABLE 2.1.2
Assay parameters for the tested protein kinases.

| | Kinase | Kinase | ATP | | Substrate |
|---|---|---|---|---|---|
| Kinase | Conc. | Conc. | Conc. | | Conc. |
| Name | (ng/50 μL) | (nM*) | (μM) | Substrate | (μg/50 μL) |
| SIK2 | 3 | 1 | 1.0 | RBER-CHKtide | 2 |
| SIK3 | 50 | 15.9 | 1.0 | RBER-CHKtide | 2 |

*Maximal molar enzyme assay concentrations, implying enzyme preparations exclusively containing 100% active enzyme

The reaction cocktails were incubated at 300C for 60 minutes. The reaction was stopped with 50 uL of 2% (v/v) H3PO4, plates were aspirated and washed two times with 200 μL 0.9% (w/v) NaCl. Incorporation of 33Pi was determined with a microplate scintillation counter (Microbeta, Wallac). All assays were performed with a BeckmanCoulter/SAGIAN™ Core System.

### Example 2.2 [Comparative]: Inhibition of SIK2 and SIK3 and Other Kinases by Compounds Having Diverse R⁶-Equivilent Moieties

It has been demonstrated that a surprisingly diverse set of heterocyclic R⁶ moieties, when used in a corresponding back-pocket binding position of another kinase-inhibiting small molecule scaffold based on the approved drug dasatinib (compound A8 herein), maintained, and even changed the selectively profile of, inhibition over a wide range of kinases (PCT/EP2019/078751; to the present applicant). Indeed, the range of heterocyclic R⁶ moieties tested and demonstrated as kinases inhibitors include those shown in FIG. 4 B to D. Such compounds were synthesised as described in Example 1 of PCT/EP2019/078751 (such example, incorporated herein by reference), and their biochemical kinase-inhibitory, in-vitro cellular and in-vivo activity were described in Example 2 to 5 (biochemical activity), Example 9 (in-vitro cellular activity) and Example 8 (in-vivo activity) thereof (such examples of PCT/EP2019/078751, incorporated herein by reference).

In particular, the IC50 of various of these compounds against various kinases is shown here Table 2.2.1. Example 3 of PCT/EP2019/078751 (such example, incorporated herein by reference) also demonstrates single point (1 uM) inhibition assays (in duplicate) over a diverse set of 320 wild-type protein kinases (“Kinase Profiler”; ProQinase, Freiburg, Germany), confirming that modification of this R⁶-equivilent moieties can surprisingly change the selectively profile of a given compound to this panel of kinases.

TABLE 2.2.1
Inhibition of various kinases by diverse R⁶ moieties.

| Com- | Kinase: IC50 (nM) | | | | |
|---|---|---|---|---|---|
| pound | ABL | SRC | SIK1 | SIK2 | SIK3 |
| A8 | ~1 to 2 | ~1 to 2 | ~1 to 2 | ~3 to 4 | ~5 to 7 |
| B3 | ~1 to 2 | ~1 to 2 | ~30 | ~60 | ~250 |
| C1 | 13.0 | <1.50 | 132 | 641 | 867 |
| C2 | 14.4 | <1.50 | 177 | 427 | 1100 |
| C3 | 39.7 | 1.59 | 571 | 1,540 | 2,780 |
| C4 | 17.7 | <1.50 | 32.7 | 130 | 677 |
| C5 | 28.2 | <1.50 | 30.6 | 119 | 340 |
| C6 | 14.7 | <1.50 | 336 | 385 | 987 |
| C7 | 5.02 | <1.50 | 13.4 | 17.7 | 52.3 |
| C8 | 296 | 22.8 | 2,800 | 4,440 | 9,730 |
| C9 | 27.4 | 6.44 | 169 | 232 | 800 |
| C10 | 4.30 | <1.5 | 302 | 138 | 493 |
| C11 | 4.43 | <1.5 | 302 | 470 | 1,510 |
| C12 | 391 | 73.7 | 1,710 | 1,940 | 8,850 |
| D1 | NT | NT | NT | ~10 to 15 | ~40 to 45 |
| D7 | NT | NT | NT | ~2 to 5 | ~15 to 20 |
| D8 | NT | NT | ~20 to 25 | ~25 to 30 | ~150 |
| D9 | NT | NT | ~10 to 15 | ~10 to 15 | ~60 |

NT = not tested

### Example 3 Kinase Selectively of Compounds of Formula (I)

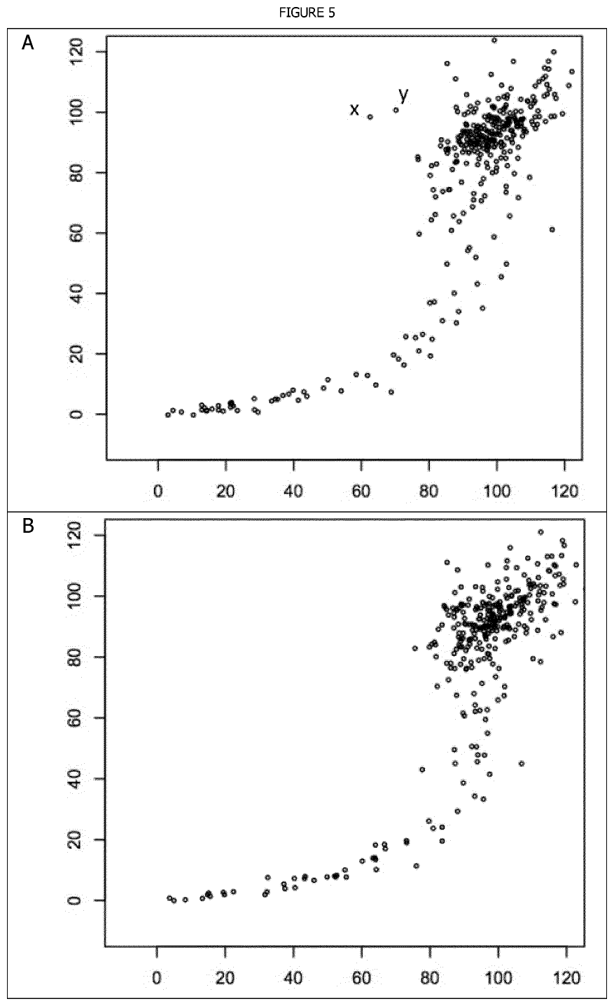

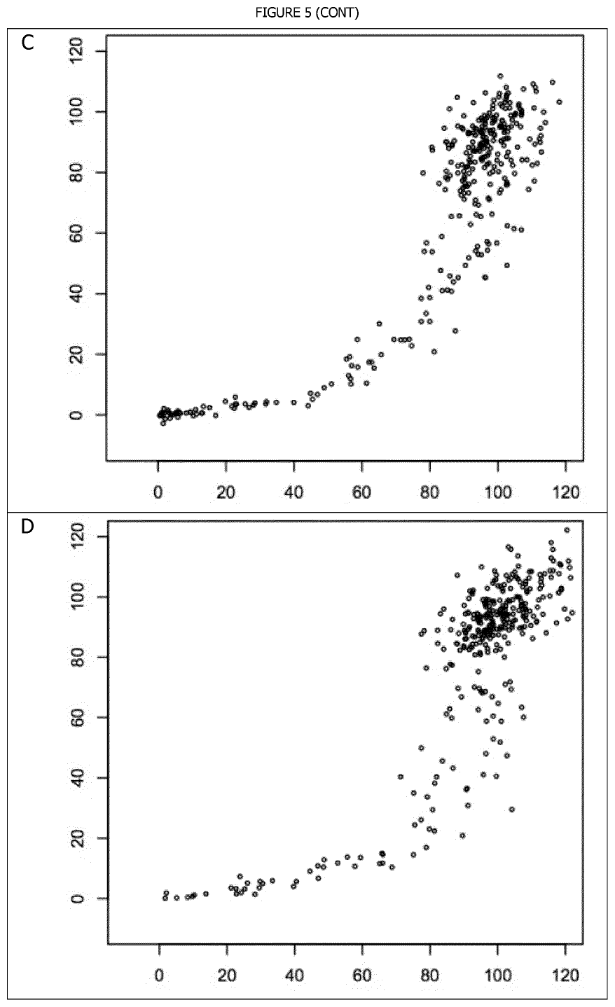

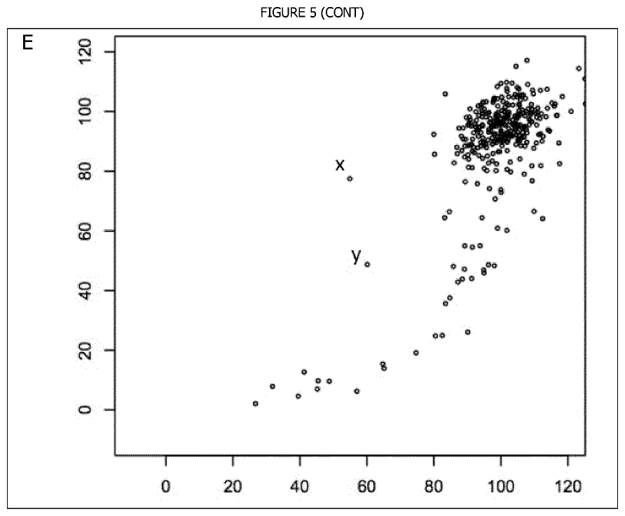

Several compounds of Formula (I) described herein were also tested in single-point (0.1 uM and 1 uM) inhibition assays over a diverse set of 335 wild-type protein (ProQinase “Kinase Profiler”; ProQinase, Freiburg, Germany), in which the residual activity of each kinase was determined. Firstly, the residual activity of each kinase was compared between the two concentrations of each compound tested and by scatter plot analysis were found to be essentially comparable (FIG. 5 A to E), especially for the kinases significantly inhibited. Secondly, the residual activity of each kinase was compared to other compounds tested (in particular, against the prior art compounds PY1) by use of scatter plots.

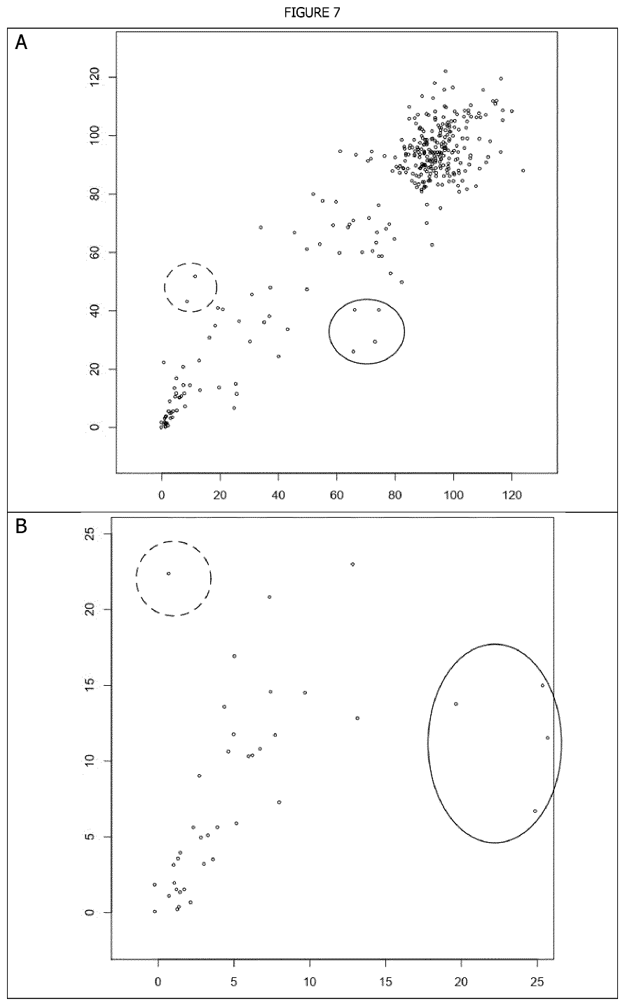

Of the compounds of Formula (I) tested, although compound AA1 had the most similar overall kinase profile to the prior art compound PY1 (FIG. 6 A), it still showed surprisingly different inhibition of particular kinases such as BLK, SIK3 and TEC (FIG. 6 B). Compound AA11 showed a substantially different profile of inhibition of the kinases compared to the prior art compound PY1; for example with the following kinases being inhibited more strongly by compound AA11 than by PY1: BRAF, NEK2, PRK2, PKC, and in particular KIT, RIPK2, ABL2 and PDGF-alpha, and with the following kinases being inhibited less strongly by compound AA11 than by PY1: BMX, TEC and in particular BTK (FIGS. 7 A and B).

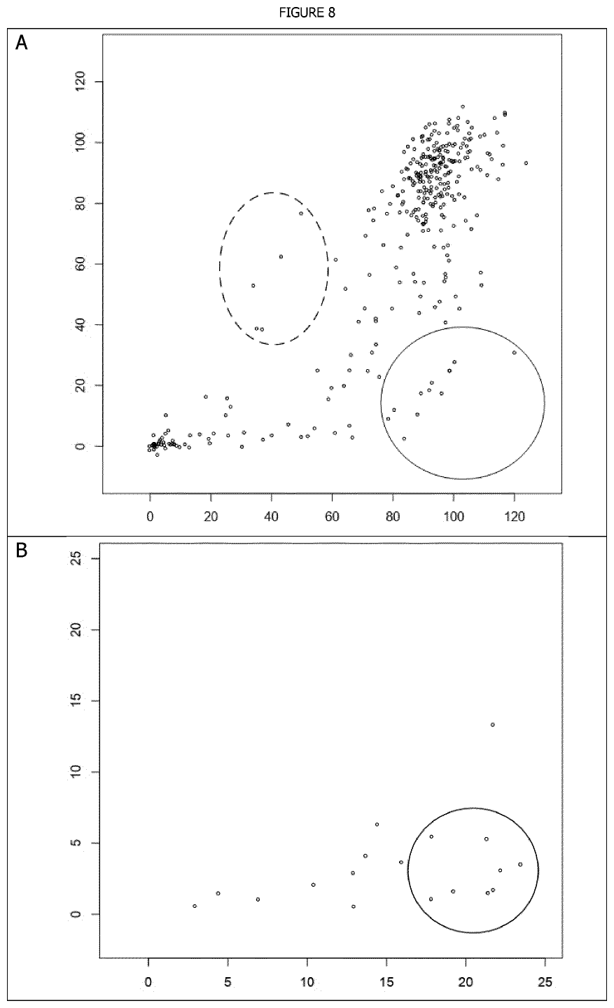

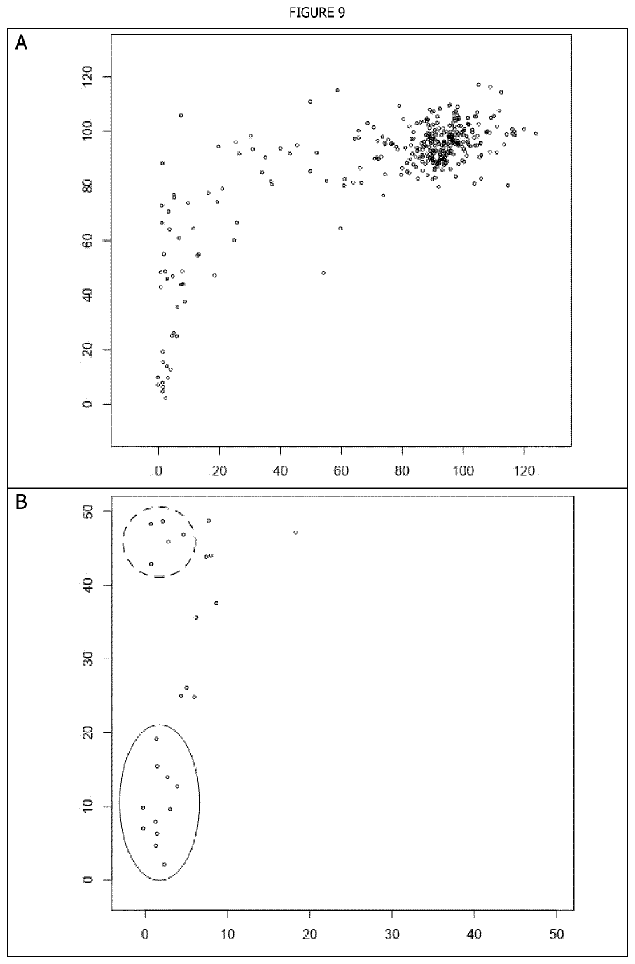

Very surprisingly, were the significantly different kinase profiles of compounds AA3 and AA5 compared to the prior art compound PY1. Overall, compound AA3 was a potent inhibitor of surprisingly more kinases than prior art compound PY1 (FIG. 8A); and in contrast, compound AA5 potently inhibited far fewer kinases than prior art compound PY1 (FIG. 9A). In Particular, there were certain relevant kinases that were significantly differently inhibited. For example, the following kinases were inhibited more strongly by compound AA3 than by PY1: TAOK2, SYK, TYRO3, ACVR2B, MEKK2, AXL, ITK, MAP3K11, TRKA, MERTK, ZAP70, and MEKK2, and in particular CSF1R, HCK, TXK, YES, LCK, SRC, EPHA1 and FGR, and with the following kinases being inhibited less strongly by compound AA3 than by PY1: SRMS, NLK, RIPK5, LTK and ALK (FIGS. 8 A and B). In contrast, far fewer kinases were potently inhibited by compound AA5 than by PY1, and many kinases were inhibited to only a limited extent compared to by PY1, for example kinases FYN, BTK, EPHB2, LCK and CSK. However, compound AA5 remained a potent inhibitor of a number of relevant kinases such as: TXK, ERBB4, EPHB1, FRK, BRK, EPHA4, ACK1, EGFR, EPHA1 and SIK1, as well as being reasonably potent against CSF1R (FIGS. 9 A and B).

These data confirm that although compounds of Formula (I) are demonstrated to be kinase inhibitors, seemingly minor structural changes can lead to surprising changes to the profile of kinases inhibited by such kinases. Accordingly, a judicial selection of substitutions to Formula (I) and/or compounds thereof, can lead to drug candidate having a particularly desired inhibitory profile of those kinases desired to be (more strongly) inhibited, and those not to be (so strongly) inhibited in order to treat the applicable disorder, disease or condition.

Subsequent IC50 testing of one or more kinases (in particular, those showing as outliers) is conducted to further investigate these differences.

For example, compounds of Formula (I) are tested for their inhibitory activity against several other kinases (eg AXL, EPHA1 EPHA3, ITK, MEKK2, LCK, SRC, ABL, MEKK3, MERTK. TYRO3, ZAP70, NEK2, BRAF, KIT, CSF1R, HCK, BTK, BLK, BRK, TEX and/or TXK), or mutants thereof).

The IC50 of each compound against these kinases is tested analogously to the IC50 assay described in Example 2.1 (in particular, see Table 2.1.2), but using the applicable kinase and its associated peptide substrate.

### Example 4: Sensitisation of Tumour Cells to In-Vitro TNF Attack by Compounds of Formula (I)

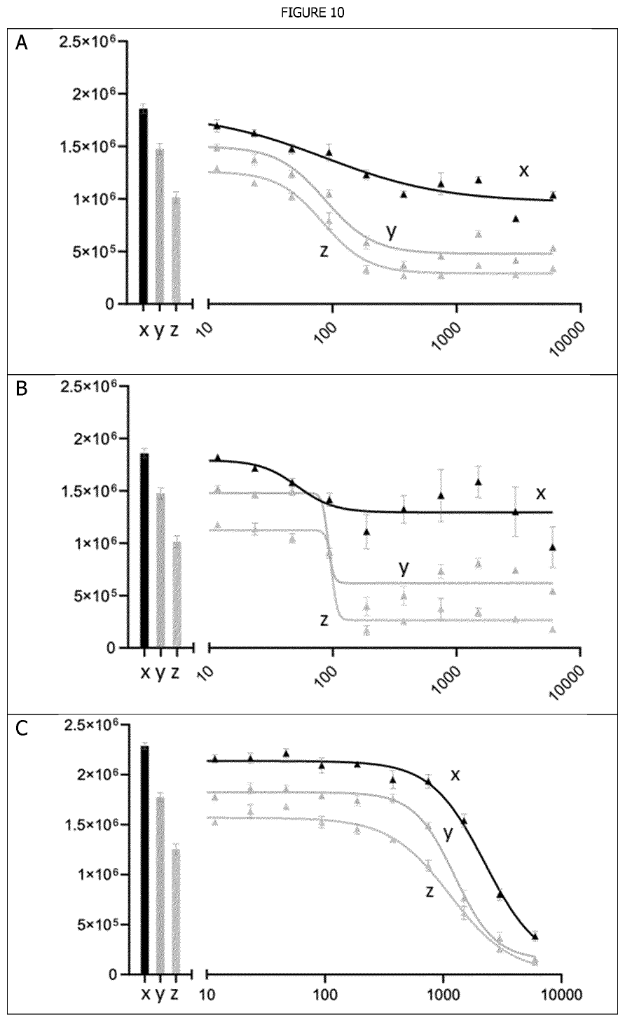

The inventors demonstrated a surprising sensitisation of tumour cells to the cytotoxic effects of recombinant TNF using a TNF-sensitised cell viability assay (Table 4.1). Indeed, human HCT116 tumour cells were surprisingly sensitised to the cytotoxic effect of TNF by compounds of Formula (I) (eg, those with a fluorinated R6 moiety) such as AA6, AA7, AA8 and AA9, which exhibited a much stronger effect compared to the prior art compound PY1 (for example, FIG. 10 A to C). Accordingly, those compounds also sensitised human PANC tumour cells to the cytotoxic effects of TNF, with AA6, AA7 and AA8 again exhibiting a stronger effect than PY1. In addition, those compounds also showed sensitising effects, comparable to PY1, in the murine MC38 tumour cell line.

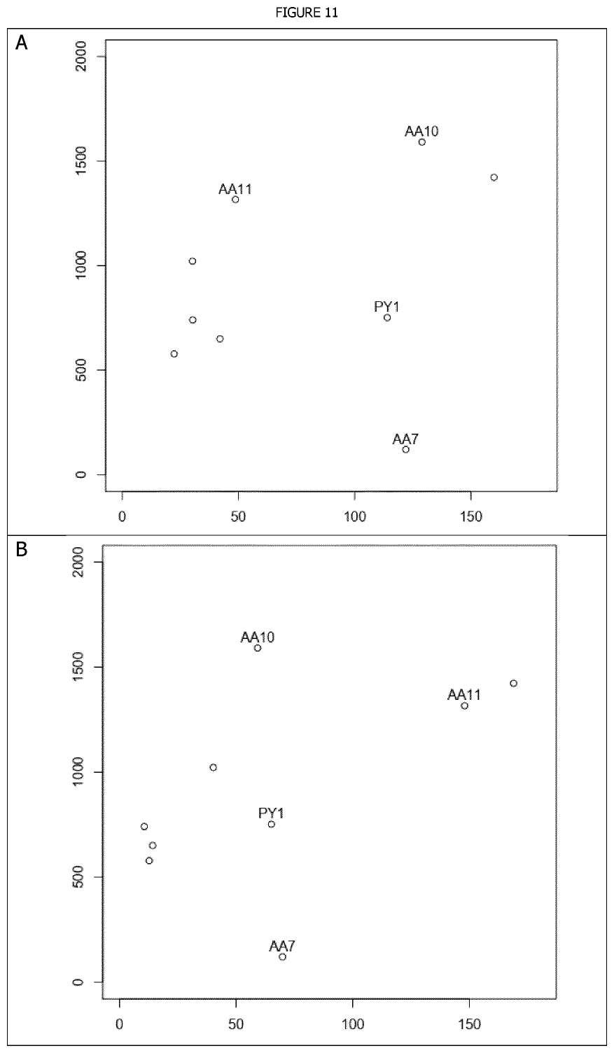

Indeed, when normalised against the respective EC50 value at Ong/mL, the EC50 of TNF-sensitisation of tested compounds of Formula (I) against each cell line generally correlated (data not shown) with the IC50 of such compounds against SIK3 (and SIK2), especially for MC38 cells exposed to 100 ng/mL rMuTNF, and all differed from that of the prior art compound PY1 (FIGS. 11 A and B). However, compound AA7 was surprisingly potent at sensitising cells to the cytotoxic effect of TNF. Also, of note is that compounds AA10 and AA11 exhibited similar effects of TNF sensitisation across all cell lines, yet had approximately-inverted inhibitory activity against SIK3 and SIK2 (See Table 2.1.1).

TABLE 4.1
Sensitisation to TNF-mediated tumour cell killing by YKL-like compounds having a fluorinated R6 moiety)

| | HCT116 IC50 | PANC IC50 | MC38 IC50 |
|---|---|---|---|
| Com- | (at 10 ng/ml | (at 100 ng/ml | (at 100 ng/ml |
| pound | rHuTNF) | rHuTNF) | rMuTNF) |
| PY1 | ++ | ++ | ++ |
| AA1 | NT | NT | + |
| AA2 | NT | NT | ++ |
| AA3 | NT | NT | ++ |
| AA6 | ++++ | +++ | ++ |
| AA7 | ++++ | +++ | +++ |
| AA8 | ++++ | +++ | ++ |
| AA9 | ++++ | ++ | ++ |
| AA10 | + | + | + |
| AA11 | / | / | + |
| AA12 | / | ND | + |
| AA13 | / | / | + |

++++ = <100 nM;
+++ = 100-500 nM;
++ = 500-1000 nM;
+ = 1000-1500 nM;
/ = >1500 nM;
NT = not tested;
ND = not determinable

Typically, those compounds of Formula (I) tested were less toxic against the cell lines than the prior art compound PY1 when tested (eg, in the absence of TNF), especially against the human cell lines such as HCT116 (data not shown). Indeed, compounds AA5 and AA6 were essentially non-toxic against all cell lines tested in the Ong/mL TNF assay conditions (MC39, HCT116 and PANC1), even up to concentrations of 10 uM (data not shown).

Other compounds of Formula (I) (such as those of Formulae (VIII)), for example those shown in FIGS. 3 A and B are synthesised in Example 1.2, are similarly tested for their ability to sensitise tumour cells o TNF-mediated killing.

The assay demonstrating sensitisation of HCT116 cells to TNF-mediated killing is conducted as follows. Cell viability of HCT116 tumour cells was measured using the CellTiter-Glo (CTG) luminescent cell viability assay (Promega, Madison, USA) according to the manufacturer's protocol. In short, 1×10³ HCT116 cells were seeded in a 384-well plate for 24 h and subsequently treated with different concentrations of compounds and 10 ng/ml rHuTNF for 72 h at 37° C. and 5% CO2. After incubation, CTG reagent was added to the wells and cells were lysed for 10 min. Read-out was performed using the Tecan reader with 0.1 sec counting time. TNF-induced cell killing in human PANC1 and murine MC38 tumour cells was measured analogously to the assay in human HCT116 cells, except that for the PANC1 assay 100 ng/ml rHuTNF and for the MC38 assay 100 ng/ml rMuTNF were used.

### Example 5 [Prophetic]: Inhibition of NFKB Activity and HDAC4 Phosphorylation by Compounds of Formula (I)

The investigators also test inhibition of TNF-induced activity of NFkB in MC38 and PANC1 cells, as well as inhibition of phosphorylation of HDAC4, the key mediator of NFkB activity.

TNF-induced NFKB activity in PANC-1 cells is measured using NFKB-dependent luciferase activity. PANC-1 clones were generated to express luciferase under the control of a NFKB promotor. NFKB reporter PANC-1 cells (1,250 per well) are seeded in 384-well plates for 24 h. Afterwards, cells are treated with different concentrations of compounds for one hour at 37° C. and 5% CO2 before addition of 10 ng/ml rHuTNF. After 7 h incubation, cells are lysed, and luciferase activity is measured as before. TNF-induced NFKB activity in MC38 cells is measured analogously to the assay in PANC-1 cells, except that 10 ng/ml rMuTNF is used.

HDAC4 phosphorylation levels in PANC-1 cells are measured using a Meso Scale Discovery (MSD) assay. PANC-1 cells (6×10⁴) are seeded in a 96-well plate overnight and subsequently treated with different concentrations of compounds of Formula (I) (in the presence of 10 ng/mL rHuTNF) for 3 h at 37° C. and 5% CO2. Whole cell lysates are generated using RIPA lysis buffer (Thermo Scientific) and incubated on GAM plates coated with anti-total HDAC4 antibody (Abcam ab12171) overnight at 4° C. Afterwards, phosphorylated HDAC4 is detected using the pHDAC4 antibody (CST #3443). ECL signal is measured using an MSD reader.

### Example 6: [Prophetic] In-Vitro Cell-Based Anti-Leukaemia Efficacy of Kinase Inhibitors, Especially Anti-MPAL Activity of Compounds of Formula (I)

The inventors test the anti-leukaemia activity of compounds of Formula (I) disclosed herein againsta particular subset of acute myeloid leukaemia (AML) cell lines (Oncolead, Karlsfeld, Germany). Of specific interest in this subset of cell lines are those that are described by others to be positive for phosphorylated myocyte enhancer factor 2C (MEF2C) protein, such as KASUMI-1, MOLM-13 and MV4-11 (Tarumoto et al 2020); or in contrast cell lines that are described by others to be pMEF2C-negative cell lines such as HL-60 and HEL, or control PBMCs.

Expression of the transcription factor MEF2C is one characteristic of mixed phenotype acute leukaemia (MPAL) (also known as “mixed lineage leukaemia”, MLL). Other characteristics of MPAL include: (i) the presence of a human chromosomal translocation at 11q23; (ii) the presence of a rearrangement of the lysine methyltransferase 2A (KMT2A) gene; (iii) the presence of, or an amount of, a KMT2A fusion oncoprotein; and/or (iv) the presence of a mutation in the K-RAS proto-oncogene GTPase (KRAS) gene and/or in the RUNX family transcription factor 1 (RUNX1) gene (Slany 2009; Schwieger et al 2009; Laszlo et al 2015; Meyer et al 2018; Tarumoto et al 2020), and in particular the expression of MEF2C is controlled by HDAC4 as a cofactor, which in turn can be retained in the cytoplasm when phosphorylated by the kinase SIK3.

The compounds of Formula (I) are inhibitors of SIK3 (and SIK2), and hence the inventors test whether such compounds are suitable for use in treatments for proliferative disorders (such as MPAL) characterised by expression of the transcription factor MEF2C, and/or is characterised by: (i) the presence of a human chromosomal translocation at 11q23; (ii) the presence of a rearrangement of the lysine methyltransferase 2A (KMT2A) gene; (iii) the presence of, or an amount of, a KMT2A fusion oncoprotein; and/or (iv) the presence of a mutation in the K-RAS proto-oncogene GTPase (KRAS) gene and/or in the RUNX family transcription factor 1 (RUNX1) gene.

Therefore, compounds of Formula (I) herein are tested for their potency to kill cancer cell lines that overexpress MEF2C, such as KASUMI-1 and MV4-11 cell lines.

Compounds of Formula (I) are also investigated in patient derived xenograft (PDX) assays, analogously to Frismantas et al 2017 (Blood 129:e26). Briefly, primary human leukaemia cells are recovered from bone marrow aspirates of T-ALL or MPAL patients and used in PDXs generated as previously described (Schmitz et al 2011, Bloood 118:1854). In addition, such compounds are assessed against primary T-ALL/MPAL cells as cell co-cultures on hTERT-immortalised primary bone marrow MSCs (Bonapace et al 2014, Oncotarget 5:11501).

### Example 7: ADMET Properties of Compounds of Formula (I)

The inventors observed that the compounds of Formula (I), when tested in ADMET assays, exhibit favourable ADMET properties, which typically were superior in terms of mouse hepatocyte stability and cell efflux ratio compared to the prior art compound PY1. These data, together with their surprising biochemical activity and selectively, can be considered to represent a viable lead-series for lead optimisation/lead selection and/or drug development activities (Table 7.1 and Table 7.2).

To determine the drug-like properties of certain of the compounds of Formula (I), the inventors test compounds in several in-vitro ADMET (absorption, distribution, metabolism, excretion, toxicity) assays, including solubility, plasma-protein binding, protein stability and stability in human and mouse liver microsomes and hepatocytes, and CaCo2 permeability and efflux, and hERG inhibition assays.

TABLE 7.1
Comparable stability and solubility of kinase inhibitors of Formula (1) compared to PY1

| | | Compound | | | |
|---|---|---|---|---|---|
| Assay | Parameter | PY1 | AA1 | AA2 | AA3 |
| Solubility | Kinetic solubility | 3.6 | 6.9 | 9.1 | 9.1 |
| | (μM) | | | | |
| Human Liver | Half-life (min) | 154 | 18 | 12 | 16 |
| microsomal | Intrinsic clearance | 4.5 | 37.3 | 57.7 | 42.2 |
| stability | (uL/min/10e6 | | | | |
| | cells) | | | | |
| Mouse liver | Half-life (min) | 87 | 45 | 10 | 20 |
| microsomal | Intrinsic clearance | 7.9 | 15.5 | 69.8 | 34.2 |
| stability | (uL/min/10e6 | | | | |
| | cells) | | | | |
| Human | Half-life (min) | 644 | 658 | 186 | 42 |
| hepatocyte | Intrinsic clearance | 1.1 | 1.1 | 3.7 | 16.3 |
| stability | (uL/min/mg) | | | | |
| Mouse | Half-life (min) | 75 | 104 | 82 | 108 |
| hepatocyte | Intrinsic clearance | 9.2 | 6.7 | 8.4 | 6.4 |
| stability | (uL/min/mg) | | | | |
| Human plasma- | Percentage | 88.2 | 90.7 | 88.6 | 87.6 |
| protein binding | unbound (%) | | | | |
| | Recovery at 5 h (%) | 53 | 32 | 32 | 34 |
| Murine plasma | Percentage | 95.2 | 95.6 | 93.7 | 93.3 |
| protein binding | unbound (%) | | | | |
| | Recovery at 5 h (%) | 42 | 40 | 38 | 45 |

Also, compounds of Formula (I) exhibited high CaCo2 A<B permeability, with an indication of some efflux but with efflux ratios of 2.4-2.9, compared to a higher A<B permeability of 3.2 for the prior art compound PY1 (Table 7.2).

TABLE 7.2
Mean efflux ratios of kinase inhibitors of Formula (I) compared to PY1 in a CaCo2 assay

| | Compound | | | |
|---|---|---|---|---|
| Parameter | PY1 | AA1 | AA2 | AA3 |
| A > B(10 EE-06 cm/s) | 9.5 | 10.5 | 12.9 | 8.4 |
| Efflux Ratio | 3.2 | 2.4 | 2.7 | 2.9 |

ADMET testing is conducted by Intonation Research Laboratories (Hyderabad, India), according to their applicable standard operating procedures.

The Kinetic solubility assay was conducted as follows. 4 uL compound of the 50 mM DMSO stock was added to deep well plate containing 396 uL PBS buffer (pH 7.4). The DMSO content in the sample was 1.0%, and the compound concentration 500 μM. The sample plate was vortexed at 800 rpm for 24 h at RT on a thermomixer. At the end of the incubation period, the sample plate was centrifuged at 4000 rpm for 10 mins. 3 uL of supernatant was transferred to well containing 297 uL of ISTD in Acetonitrile water:50:50, v/v and analysed in LC-MS/MS against a calibration curve (CC).

Briefly, human and liver microsomal stability was determined by pre-incubation of 2.5 uL compound and 75 uL of the respective liver microsomes in potassium phosphate buffer (pH 7.4) at 37° C. After adding of NADPH as cofactor and further incubation at 37° C. for 60 min, acetonitrile containing internal standard was added, and the sample was vortexed for 5 min at 1200 rpm and then centrifuged 10 min at 4000 rpm. The supernatant was diluted 2-fold with water, and intrinsic clearance and half-life were determined using LC-MS/MS.

Determination of human and mice hepatocyte stability was conducted as follows. 200 uL of the respective hepatocyte cell suspension (2×10e6/ml) was preincubated at 37° C. for 10 min. Then, 200 uL of a 2 uM working stock of the test compound was added to the hepatocyte cell suspension, and the mixture was further incubated at 37° C. and 500 rpm. The reaction was stopped at 30, 60 and 120 min by precipitating 50 uL of the incubation mixture with 200 uL of acetonitrile. Samples were vortexed for 5 min at 1200 rpm and centrifuged at 4000 rpm for 10 min. Intrinsic clearance and half-life were analysed using LC-MS/MS.

Human and mice protein plasma binding was determined using the Plasma Protein Binding by RED Method. Briefly, 200 uL of the respective plasma containing the test compound (10 uM) was spiked into the donor well (RED Chamber) of the insert. 350 uL of PBS was spiked into receiver well (white chamber). The plate was incubated at 37° C. in a thermomixer at 400 rpm for 5 hours. The samples were matrix equilibrated with opposite matrix (25 uL blank buffet/Plasma). Matrix matched samples were precipitated with 200 uL of Acetonitrile containing Internal standard. Samples were vortexed at 1000 rpm for 5 min and centrifuged at 4000 rpm for 10 min. The supernatant was diluted 2 folds with water and % bound and unbound protein and % recovery at 5 h was determined by LC-MS/MS.

To determine apparent permeability of the compounds an assay using a Caco-2 cells monolayer (cultured for 21d) in a 24-well format (poly carbonate high pore density plate with 0.4 μm pore size and a 0.7 cm2 active membrane area; Millipore) was performed as followed. 800 uL of DMEM was added to the basal compartment of the 24 well-plate and 60000 CaCo2 cells/well were seeded in the apical wells and incubated in a CO2 incubator at 37° C. for proliferation of cells. Apical to Basal permeability was determined by adding 400 uL of test compound to the apical well and 800 uL of HBSS buffer with 2% BSA to the basal well. 25 uL of basal sample was collected after 120 min and processed as stated below. Basal to Apical permeability was determined by adding 800 uL of test compound to basal well and 400 uL of HBSS buffer with 2% BSA to apical well. 25 uL of apical sample was collected after 120 min and processed as stated below. The samples were processed by diluting the donor samples 1:1 with HBSS containing 2% BSA and the receiver samples 1:1 with HBSS buffer. Samples were then precipitated with 200 uL of acetonitrile containing internal standard, vortexed for 5 min at 1000 rpm, and centrifuged at 4000 rpm for 20 min. 150 uL of supernatant was diluted with 150 uL of water and submitted for LC-MS/MS analysis, and Bi-directional (A-B and B-A) apparent permeability (Papp) was calculated.

LC-MS/MS was performed for all assays using a C18 column (50*4.6 mm, 5 um) for the CaCo2 assay, or a C18 column (50*4.6 mm, 4 um) for the other assays. The mobile phases used were 10 mM Ammonium Acetate with 0.1% Formic acid (aqueous) and 100% Acetonitrile (organic).

hERG inhibition assays are conducted by Charles River Inc., at their UK Discovery site (Cambridge, UK). Briefly, the potential for test compound to inhibit the hERG potassium channel is determined using the Charles River ChanTest® hERG-HEK stably transfected cell line on the Sophion Qube automated electrophysiology platform. The assay is performed at room temperature and recordings of the hERG tail current from individual cells are made using single-hole QChips. The cells are held at a voltage of −80 mV and then stepped to +40 mV for 2 seconds before stepping to −40 mV for a further 2 seconds, this represents 1 experimental sweep. This voltage protocol is applied every 15 seconds for the duration of the experiment. Both the vehicle and 2nd compound addition periods are applied for 20 sweeps. The 1st compound addition period is applied for 10 sweeps. The potency (IC50) of the test compound to inhibit the hERG channel is determined from a concentration-response curve generated from up to 8 test compound concentrations with up to 4 replicates per concentration. The compound concentration is added to the test well twice to assure complete exchange of the external buffer with the test compound. In total, compound is applied to the well for 450 seconds. Quality control filters used are: whole-cell membrane resistance≥200 MOhm, and vehicle current amplitude≥400 pA. The analysis methodology comprised: The peak tail currents evoked by the step to −40 mV are measured for the analysis of the percentage inhibition by test compounds. The peak tail currents are first normalised to the vehicle addition (0.3% DMSO) in the same well. The percent inhibition versus Log 10 compound concentration data is plotted and the IC50 determined using a sigmoidal dose response equation.

### Example 8: Pharmacokinetic and Tolerability Studies of Compounds of Formula (I) Disclosed Herein

Screening PK properties of compounds of Formula (I) are additionally determined, including plasma and free plasma concentration, as well as Cmax. For example, the free concentration of compound after a dose of eg 30 mg/kg po (per os) administered mice can be determined for certain time points after administrations.

To further investigate PK properties of kinase inhibitors of Formula (I), the compounds are tested for in vivo drug exposure and potency in a full pharmacokinetic study with oral administration (per os, po).

The experimental procedure and the determination of the pharmacokinetic parameters are investigated after single administration of each compound per gavage in male CD1 mice (Intonation Research Laboratories (Hyderabad, India)). The compounds are diluted in 5% DMSO+5% Solutol+90% water. Plasma level (eg sampling 0.25 h; 0.5 h; 1 h; 1.5 h; 2 h; 4 h; 6 h; 12 h; 24 h after drug administration) of the compounds are analysed by LC-MS/MS and pharmacokinetic parameters are determined using non-compartmental analysis and nominal dose levels of eg 30 mg/kg po and 1 mg/kg iv. Three animals are analysed per time point and each mouse is used for three plasma samplings in total.

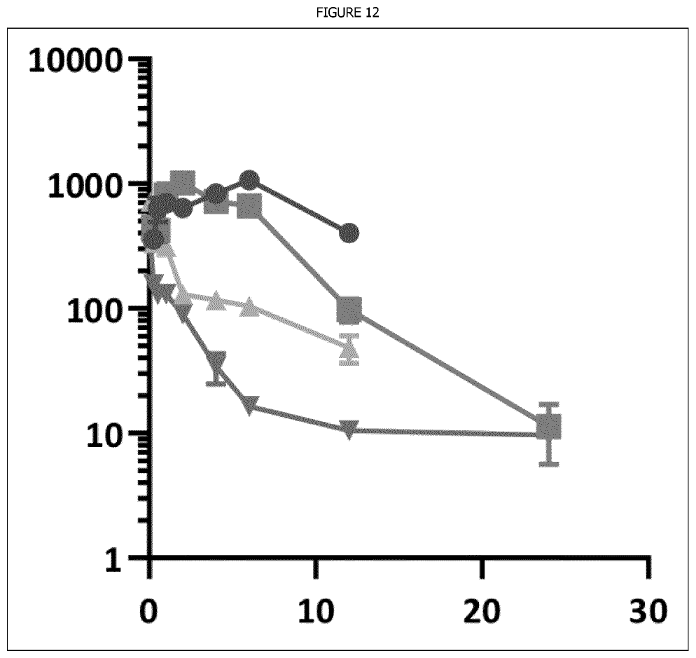

No PY1 (the prior art compound YKL-05-099) was detectable after 24 h in either 30 mg/kg po or 1 mg/kg iv administration. Surprisingly however, compound AA1 was detectable after 24 hours in both 30 mg/kg po or 1 mg/kg iv administrations (FIG. 12). These data are an indication of superior in vivo PK properties for compounds of Formula (I) such as compound AA1 (or other compounds such as AA6 or AA7).

Also, the maximal tolerable dose (MTD) of the kinase inhibitors disclosed herein is investigated. This is done by once (QD) or twice daily (BID) administration of different concentrations (eg 3 and 100 mg/kg) of the compounds by gavage in female C57Bl/6 mice for 7 consecutive days. The compounds are diluted in 4% (v/v) DMSO, 72% (v/v) propylene glycol+24%(v/v) dd-water. The C57BL/6 animals are observed for seven days for any signs of intolerance (posture, vocalization, ease of handling, lacrimation, chromodacryorrhea, salivation, intact fur/coat, rearing, arousal, piloerection, normal motor movements, tail pinch, diarrhoea). Additionally, body weight is monitored and plasma-level of the compounds is measured by LC-MS/MS.

### Example 9: [Prophetic] In-Vivo Anti-Cancer (Solid Tumour) Efficacy of Compounds of Formula (I)

The anti-cancer activity of compounds of Formula (I) (eg AA6 and AA7) against solid tumours are investigated in an in vivo syngeneic mouse model. Dosage and administration regimens for the tested compounds of Formula (I) are adapted according to their respective SIK3 kinase inhibition and DMPK properties (see previous examples).

The study is conducted by implanting murine colorectal carcinoma MC38 cells subcutaneously in the flanks of C57Bl/6N mice and treating the mice with the test compounds (at doses of between eg 2.5 mg/mL/day up to 50, 60 or 100 mg/Kg/day*). In detail, female C57Bl/6N mice (4-6 weeks old), are implanted with of 1×10⁶ MC38 cells (100 μl in PBS). Mice are randomised into treatment groups after reaching 150 mm3 tumour volume, and treatment groups may, for example, comprise those as set forth in Table 9.1, with treatment starting within 24 h of randomisation.

TABLE 9.1
Example treatment groups.

| | | Total daily dose | Dosing | | | Number |
|---|---|---|---|---|---|---|
| Group | Treatment | [mg/kg/d] | days | Route | Vehicle | of mice |
| | Rat IgG2a isotyp | 10 mg/Kg | daily | i.p. | Vehicle | 15 |
| | control (clone 2A3) | | | | | |
| | Anti-PD-1 | 10 mg/Kg | daily | i.p. | Vehicle | 15 |
| | (clone RMP1-14) | | | | | |
| 1 | Vehicle | — | daily | po | — | 15 |
| 2 | AA6 | eg 60 mg/Kg*# | daily# | po | Vehicle** | 15 |
| 2 | AA7 | eg 30 mg/Kg* | daily | po | Vehicle** | 15 |

*Based on last bodyweight measurement;
#For at least 3 weeks, up to about 5-8 weeks. Lower dosage (eg, 10, 15 or 20 mg/Kg) can be adapted according to their ADMET/PK properties.
** = 10% DMSO + 5% Solutol + 40% PEG + 45% water

Mice are measured for body weight and tumour volume (mm3) by calliper measurement twice weekly for up to 8 weeks until termination criteria (tumour volume>2000 mm3) is reached.

Additionally, 5 mice per group are sacrificed after day 9 of first treatment to analyse tumour and blood samples for various immune-response markers, for example those set forth in Table 9.2 below, (as well as using Aqua Zombie (BioLegend) to determine live/dead cells).

Briefly, peripheral blood samples are collected (however, the day before sacrifice) from these mice into heparin-precoated tubes by making an incision in the tail of the mice. After treating the blood samples with ammonium-chloride-potassium (ACK) lysing buffer (Life Technologies, Cat. A10492-01), the cells are stained with fluorochrome-conjugated mAbs to one or more of the example immune-phenotyping markers (eg, Table 9.2).

For immune-phenotyping of the tumours, tumours are surgically removed on the day of sacrifice with a scalpel and then divided in half. One part of the tumour is fixed in 4% paraformaldehyde (PFA) for immunohistochemistry for one or more of the example immune-phenotype markers. For IHC, PFA-fixed tumour tissues are embedded into paraffin blocks and cut at 4 mm thickness. After fixing on glass slides and antigen-retrieval step, the sections are stained with anti-CD8 antibody and counterstained with Mayer-Hematoxylin. The counting of the tumour infiltrating CD8 T cells is done at 50× magnification starting from capsule area and counting 3 fields toward to the core of the tissue. The same process is repeated 3 times. All counts are summed, and the median calculated (as described in Hekim et al 2017, Can Imm Res 5:157).

The other half of the tumour is transferred into 1.5 mL tubes containing RPMI 1640 medium and then homogenized manually using micro tube pellet pestle. After centrifugation at 300×g, the supernatant is discarded, and cells are resuspended in RPMI medium equal to the tumour weight. Tumour homogenate is diluted 1:1 in PBS and stained with fluorochrome-labelled antibodies for one or more of the example immune-phenotype markers. For all Foxp3-specific staining (peripheral or intra-tumoural), cells are first labelled with the anti-CD4 antibody before incubation with the anti-Foxp3 antibody using an Intracellular Fixation and Permeabilization Kit as recommended by the manufacturer (eBioscience)

Two hours after the last dose, at least 100 uL of whole blood, as well as tumour tissue, of six animals in each group are analysed by flow cytometry for CD4+ and CD8+ T cells, Tregs, granulocytic and monocytic MDSCs, M1 and M2 macrophages and NK cells. Immediately following blood collection, tumours are excised and processed for analysis by flow cytometry. Tumour and blood samples are analysed for various immune-response markers (Table 9.2), as well as using a Zombie dye (BioLegend) to determine live/dead cells. Intracellular cytokines within the lymphoid panel are detected after ex vivo stimulation of T cells with PMA/ionomycin/Brefeldin A.

TABLE 9.2
Immune-phenotype markers.

| T cell panel | | Myeloid cell panel | |
|---|---|---|---|
| Target | Clone | Target | Clone |
| CD45 | 30-F11 | CD45 | 30-F11 |
| CD3e | 145-2C11 | CD3e | 145-2C11 |
| CD4 | RM4-5 | CD11b | M1/70 |
| CD8 | 53-6.7 | F4/80 | BM8 |
| CD25 | PC61 | Ly6C | AL-21 |
| FoxP3 | FJK-16s | Ly6G | 1A8 |
| IFNy | XMG1.2 | CD206 | C068C2 |
| GrzB | NGZB | MHC-II | 10-3.6 |
| CD69 | H1.2F3 | CD49b | DX5 |
| CD107a | 1D4B | CD335 | 29A1.4 |
| Live/dead | (Zombie Dye) | Live/dead | (Zombie Dye) |

Sample preparation for flow cytometry is conducted as follows: whole blood samples are processed by adding a ten-fold volume of ammonium-chloridepotassium (ACK) buffer at ambient temperature, mixed gently and incubated for 3-5 minutes at room temperature. Immediately after incubation, a ten-fold volume of cold PBS is added to stop the lysis reaction, and cells are pelleted at 400 g for five minutes and washed again in PBS. Mouse tumour samples are dissociated according to the manufacturer's instructions using the gentleMACS™ protocol “Tumor Dissociation Kit”. Briefly, tumours are excised, cut into small pieces (2-4 mm), placed into an enzymatic buffer and processed on a gentleMACS Dissociator, incubated for 20 minutes at 37° C. with continuous rotation. Samples are filtered through a 70 um cell strainer and rinsed twice in PBS/2.5% FBS buffer to remove enzymatic buffer. All single cell suspensions are prepared at ˜1×10e7 cells/mL in PBS and kept on ice. Ex vivo stimulation of samples is performed for T-cell marker-panel populations with PMA/ionomycin/Brefeldin A. 100 uL of single cell suspensions are added into 96-well plates, stained, and analysed with an LSRFortessa™ (BD) and analysed with FlowJo software (Tree Star, Inc.; version 10.0.7r2).

### Example 10 [Prophetic]: In-Vivo Immune-Oncology Activity of Compounds of Formula (I)

To investigate the synergistic effect of TNF-inducing therapy (eg, an anti-PD1 antibody, such as murine anti-PD1 clone: RMP1-14, BioLegend) with a compound of Formula (I) (eg, AA6 and AA7), (at doses of between eg 2.5 mg/Km/day up to 50, 60 or 100 mg/Kg/day*), a further in vivo syngeneic mouse model as described in Example 9 is conducted, but with treatment groups addressing applicable combinations (and controls), for example treatment groups which may, for example, comprise those as set forth in Table 10.1, below.

TABLE 10.1
Example treatment groups.

| | | Total daily dose | Dosing | | | Number |
|---|---|---|---|---|---|---|
| Group | Treatment | [mg/kg/d] | days | Route** | Vehicle | of mice |
| 5 | Vehicle** + | 10 mg/Kg* | 2x weekly | ip | — + PBS | 15 |
| | ratIgG2a | | | | | |
| | (control) | | | | | |
| 6 | Vehicle** + | — | daily + | po + | — + PBS | 15 |
| | anti-PD-1 | 10 mg/Kg* | 2x weekly | ip | | |
| 7a | AA6 + | eg 60 mg/Kg*# | daily# + | po + | Vehicle** + | 15 |
| | anti-PD-1 | 10 mg/Kg* | 2x weekly | ip | PBS | |
| 7b | AA6 + | eg 30 mg/Kg* | daily# + | po + | Vehicle** + | 15 |
| | anti-PD-1 | 10 mg/Kg* | 2x weekly | ip | PBS | |
| 8 | PY1 + | eg 30 mg/Kg* | Twice | po + | Vehicle** + | 15 |
| | anti-PD-1 | 10 mg/Kg* | daily# + | ip | PBS | |
| | | | 2x weekly | | | |

*Based on last bodyweight measurement;
**po = per os, oral administration via gavage, ip = intra-peritoneal
#Compound dosed for at least 3 weeks, up to between 5 and 8 weeks. Anti-PD-1 and vehicle treatment for duration. Lower dosage (eg, 10, 15 or 20 mg/Kg) or higher dosage (eg, 50, 75 or 100 mg/Kg) can be adapted according to their ADMET/PK properties.
**Propylene glycol:water 1:1

As described in Example 9: (i) mice are measured for body weight and tumour volume (mm3) by calliper measurement twice weekly for up to 8 weeks until termination criteria (tumour volume>2000 mm3) is reached; and (ii) 5 mice per group are sacrificed after day 9 of first treatment to analyse tumour and blood samples for various immune response markers (eg, as described in Example 9).

### Example 11 [Prophetic]: Formulation for and Preparation of Unit Dose Form of Compounds of Formula (I) for Oral Administration

A caplet unit dose form of a pharmaceutical composition is made, briefly as follows.

First, a tableting blend comprising the compounds of Formula (I) (eg, AA6 and AA7), is prepared by dry granulation of an amount of (AA6 and AA7) together with one or more excipients. Example excipients in the tableting blend can include a binder, such as lactose (monohydrate), microcrystalline cellulose and/hydroxypropyl cellulose, and optionally with a disintegrant such as starch. The blend may also include a lubricant such as magnesium stearate. Alternatively, the tableting blend is prepared by wet granulation followed by drying.

Second, using a rotary tablet press, the blend is filled into a suitably shaped die from above, and compressed to a porosity of between about 5% and 20% by lowering an upper punch into the die. Compression can take pace in in one or two stages (main compression, and optionally pre-compression or tamping), with compression occurring rapidly for scaled manufacturing (eg within 500 ms per caplet). The upper punch is pulled up and out of the die (decompression), and the caplet is ejected from the die.

Third, the caplet is coated using an automatic coater. The coating can comprise hypromellose, titanium dioxide, polyethylene glycol and purified water.

The tableting blend comprises an amount of the kinase inhibitor (AA6 and AA7, as used) such that each caplet is made to include a therapeutically effective amount of AA6 and AA7, and caplets of different dosages may be made to aid the administration of the correct overall dose of AA6 and AA7. For example, each caplet may include about 20 mg, 50 mg or 70 mg of (AA6 and AA7, as applicable), or may include less than these amounts such as about 5 mg, 10 mg or 40 mg of (AA6 and AA7 as applicable).

### Example 12 [Prophetic]: Production of Melanin in Human Skin by Compounds of Formula (I)

Following the methodology described by Kumagai et al (2011, PLoS ONE 6(10): e26148), compounds of Formula (I) are tested first for their ability to induce melanogenesis in B16F10 melanoma cells.

B16F10 murine melanoma cells and HEK293 cells are obtained from the American Type Culture Collection (Manassas, Va., USA). B16F10 cells are growth at 37° C. under 5% CO2 in Dulbecco's modified Eagle's medium (DMEM; high glucose) (Wako) supplemented with 10% foetal bovine serum (FBS), penicillin (100 U/mL), and streptomycin (50 mg/mL). B16F10 are seeded in 6-well plates at a density of 3.4×10**5 cells/well. After 24 h, the culture medium is replaced with fresh medium supplemented with test compound, and, after 48 h, the medium is changed again with fresh medium containing the same compound. After an additional 24 h, the cells ae harvested for a melanin or mRNA/protein assay. To measure melanin, the cells are washed twice with phosphate-buffered saline (PBS), suspended in PBS, and recovered by centrifugation at 8,000 rpm for 1.5 min. The cell pellet is suspended in 300 mL of 1N NaOH and incubated at 45° C. for 2 h, and, then, melanin extracted with a chloroform-methanol mixture (2:1). Melanin is detected with a spectrophotometer (BIO-RAD Model 680 MICRO PLATE READER; Bio-Rad, Hercules, Calif., USA) at 405 nm. A standard curve is obtained by using purified melanin (0-1,000 mg/mL). The protein concentration of the cell pellets was determined using the Bradford reagent (Bio-Rad) and used for normalisation of the melanin content.

Compounds of Formula (I) are secondly tested for their ability to recue melanogenesis I mice with an inactive melanocortin 1 receptor, using the methodology described in Mujahid et al (2017, Cell Reports 19:2177).

Briefly, a previously described mouse “red hair” model is utilised that carries the inactivating Mc1r^{e/e} mutant allele and a transgene, K14-SCF, in which stem cell factor expression is driven by the keratin-14 promoter, allowing for epidermal homing of melanocytes (D′Orazio et al 2006, Nature 443:430; Kunisada et al 1998, J Exp Med 187:1565). Albino mice harbouring a mutation in the tyrosinase gene are combined with the K14-SCF transgene (Tyr^{c/c};K14-SCF mice) and serve as controls to evaluate whether the pigmentation afforded by topical SIK inhibitor is dependent upon the canonical tyrosinase-melanin pathway. Daily application of the SIK inhibitors of Formula (I) for 7 days can cause darkening in Mc1r^{e/e};K14-SCF mice. No visible change in skin pigmentation is observed in Mc1r^{e/e};K14-SCF mice treated with vehicle or in Tyr^{c/c};K14-SCF mice treated with vehicle or SIK inhibitors of Formula (I).

In a third investigation, compounds of Formula (I) are studied for their ability to induce human skin eumelanisation, using the methodology described in WO2018/160774.

Full thickness human breast skin explants are cultured in petri dishes with a solid phase and liquid phase phenol red free DMEM medium with 20% penicillin streptomycin, 5% fungizone (Gibco®), and 10% FBS. Explants are treated daily with vehicle, HG 9-91-01 (obtained from MedChemExpress LLC, Monmouth Junction, NJ, USA) or SIK inhibitor of Formula (I). Passive application refers to simply applying the treatment to skin without further rubbing or manipulation. Mechanical application refers to application of agents to skin with further rubbing of treatment with 10 clockwise turns of a gloved cotton swab applicator. Skin is harvested, fixed, and processed for paraffin embedding. Sections are cut from paraffin blocks, and sections stained utilising haematoxylin and eosin (morphology) and Fontana-Masson (for melanin). Treatment of human skin explants with passive topical application of SIK inhibitors of Formula (I) can induce significant pigmentation without any additional treatments after 8 days (1×/day) of treatment, but no significant gross pigmentation is observed in skin treated with HG 9-91-01. Fontanna Mason staining reveals increased melanin content in skin treated with SIK inhibitors of Formula (I) and marginally increased melanin in skin treated with HG 9-91-01 as compared to control. Mechanical application of the HG 9-91-01 (by rubbing via an applicator) induces significant gross pigmentation, and increased melanin content is observed upon Fontana Masson staining of skin sections. HG 9-91-01's human skin limited penetration can thereby be at least partially overcome through mechanical application. SIK inhibitors of Formula (I) may not require mechanical application (rubbing) to induce significant human epidermal darkening after 8 days (lx/day) of treatment. Fontanna Mason staining for melanin further illustrates whether exemplary SIK inhibitors of Formula (I) induce pigmentation in the human skin explants.

1. A compound selected from the group consisting of a kinase inhibitor of the formula (I): 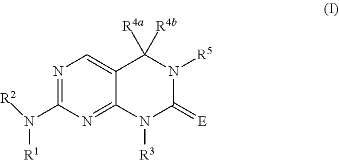
and solvates, salts, N-oxides, complexes, polymorphs, crystalline forms, racemic mixtures, diastereomers, enantiomers, tautomers, conformers, isotopically labeled forms, prodrugs, and combinations thereof;
wherein:
R¹ is -Q-R^{1a};
R^{1a} is selected from the group consisting of H, alkyl, alkenyl, alkynyl, cycloalkyl, aryl, heterocyclyl, heteroaryl, halogen, —CN, azido, —NO₂, —OR¹¹, —N(R¹²)(R¹³), —N(R¹¹)(OR¹¹), —S(O)₀₋₂R¹¹, —S(O)₁₋₂OR¹¹, —OS(O)₁₋₂R¹¹, —OS(O)₁₋₂OR¹¹, —S(O)₁₋₂N(R¹²)(R¹³), —OS(O)₁₋₂N(R¹²)(R¹³), —N(R¹¹)S(O)₁₋₂R¹¹, —NR¹¹S(O)₁₋₂OR¹¹, —NR¹¹S(O)₁₋₂N(R¹²)(R¹³), —P(O)(OR¹¹)₂, —OP(O)(OR¹¹)₂, —C(═X)R¹¹, —C(═X)XR¹¹, —XC(═X)R¹¹, and —XC(═X)XR¹¹, wherein each of the alkyl, alkenyl, alkynyl, cycloalkyl, aryl, heterocyclyl, and heteroaryl groups is optionally substituted with one or more independently selected R³⁰;
Q is selected from the group consisting of C₆₋₁₀ aryl, and 5- or 6-membered heteroaryl, wherein each of the C₆₋₁₀ aryl, and 5- or 6-membered heteroaryl is optionally substituted with one or more R³⁰ independently selected from the group consisting of C₁₋₈ alkyl, halogen, —OR¹¹, —N(R¹²)(R¹³), —SR¹¹, C(═O)R¹¹, wherein each of the C₁₋₈ alkyl, halogen, —OR¹¹, —N(R¹²)(R¹³), —SR¹¹, C(═O)R¹¹ is optionally substituted with one or more independently selected R³⁰;
R² is H;
R³ C₁₋₈ alkyl, C₃₋₁₀ cycloalkyl, C₆₋₁₀ aryl, and 5- to 8-membered heteroaryl, wherein each of the C₁₋₈ alkyl, C₃₋₁₀ cycloalkyl, C₆₋₁₀ aryl, and 5- to 8-membered heteroaryl groups is optionally substituted with one or more R³⁰ independently selected from the group consisting of C₁₋₈ alkyl, halogen, —OR¹¹, —N(R¹²)(R¹³), —SR¹¹, C(═O)R¹¹, wherein each of C₁₋₈ alkyl, halogen, —OR¹¹, —N(R¹²)(R¹³), —SR¹¹, and C(═O)R¹¹ is optionally substituted with one or more independently selected R³⁰;
optionally R¹ and R³ may join together via a group L′ to form a moiety R¹-L′-R³, preferably to form a moiety Q-L′-R³, wherein L′ is selected from the group consisting of a bond, alkylene, alkenylene, alkynylene, —(CH₂)ₚ—[Y—(CH₂)_{q}]ᵣ—, alkenylene —[Y—(CH₂)_{q}]ᵣ—, wherein p is an integer between 1 and 10, q is an integer between 0 and 6, r is an integer between 1 and 3, wherein if q is 0 then r is 1; Y is independently selected from O, S, and —N(R¹³)—; and each of the alkylene, alkenylene, alkynylene, —(CH₂)ₚ—, and —(CH₂)_{q}— groups is optionally substituted with one or more independently selected R³⁰
both R^{4a} and R^{4b} are H;
R⁵ is -L-R⁶;
L is a bond;
R⁶ is thienyl optionally substituted with one, two, or three independently selected R⁷;
R⁷ is independently selected from the group consisting of alkyl, alkenyl, alkynyl, cycloalkyl, aryl, heterocyclyl, heteroaryl, halogen, —CN, azido, —NO₂, —OR¹¹, —N(R¹²)(R¹³), —N(R¹¹)(OR¹¹), —S(O)₀₋₂R¹¹, —S(O)₁₋₂OR¹¹, —OS(O)₁₋₂R¹¹, —OS(O)₁₋₂OR¹¹, —S(O)₁₋₂N(R¹²)(R¹³), —OS(O)₁₋₂N(R¹²)(R¹³), —N(R¹¹)S(O)₁₋₂R¹¹, —NR¹¹S(O)₁₋₂OR¹¹, —NR¹¹S(O)₁₋₂N(R¹²)(R¹³), —P(O)(OR¹¹)₂, —OP(O)(OR¹¹)₂, —C(═X)R¹¹, —C(═X)XR¹¹, —XC(═X)R¹¹, and —XC(═X)XR¹¹, and/or any two R⁷ which are bound to the same atom of R⁶ being a heterocyclyl group may join together to form ═O, wherein each of the alkyl, alkenyl, alkynyl, cycloalkyl, aryl, heterocyclyl, and heteroaryl groups is optionally substituted with one or more independently selected R³⁰;
E is O;
R¹¹ is independently selected from the group consisting of H, alkyl, alkenyl, alkynyl, cycloalkyl, aryl, heteroaryl, and heterocyclyl, wherein each of the alkyl, alkenyl, alkynyl, cycloalkyl, aryl, heteroaryl, and heterocyclyl groups is optionally substituted with one or more independently selected R³⁰;
each of R¹² and R¹³ is independently selected from the group consisting of H, alkyl, alkenyl, alkynyl, cycloalkyl, aryl, heteroaryl, and heterocyclyl, or R¹² and R¹³ may join together with the nitrogen atom to which they are attached to
form the group —N═CR¹⁵R¹⁶, wherein each of the alkyl, alkenyl, alkynyl, cycloalkyl, aryl, heteroaryl, and heterocyclyl
groups is optionally substituted with one or more independently selected R³⁰;
each of R¹⁵ and R¹⁶ is independently selected from the group consisting of H, alkyl, alkenyl, alkynyl, cycloalkyl, aryl, heteroaryl, heterocyclyl, and —NH_{y}R²⁰_{2-y}, or R¹⁵ and R¹⁶ may join together with the atom to which they are attached to form a ring which is optionally substituted with one or more independently selected R³⁰, wherein each of the alkyl, alkenyl, alkynyl, cycloalkyl, aryl, heteroaryl, and heterocyclyl groups is optionally substituted with one or more independently selected R³⁰;
y is an integer from 0 to 2;
R²⁰ is independently selected from the group consisting of alkyl, alkenyl, alkynyl, cycloalkyl, aryl, heteroaryl, and heterocyclyl, wherein each of the alkyl, alkenyl, alkynyl, cycloalkyl, aryl, heteroaryl, and heterocyclyl groups is optionally substituted with one or more independently selected R³⁰; and
R³⁰ is a 1^{st} level substituent and is, in each case, independently selected from the group consisting of alkyl, alkenyl, alkynyl, aryl, heteroaryl, cycloalkyl, heterocyclyl, halogen, —CN, azido, —NO₂, —OR⁷¹, —N(R⁷²)(R⁷³), —S(O)₀₋₂R⁷¹, —S(O)₁₋₂OR⁷¹, —OS(O)₁₋₂R⁷¹, —OS(O)₁₋₂OR⁷¹, —S(O)₁₋₂N(R⁷²)(R⁷³), —OS(O)₁₋₂N(R⁷²)(R⁷³), —N(R⁷¹)S(O)₁₋₂R⁷¹, —NR⁷¹S(O)₁₋₂OR⁷¹, —NR⁷¹S(O)₁₋₂N(R⁷²)(R⁷³), —OP(O)(OR⁷¹)₂, —C(═X¹)R⁷¹, —C(═X¹)X¹R⁷¹, —X¹C(═X¹)R⁷¹, and —X¹C(═X¹)X¹R⁷¹, and/or any two R³⁰ which are bound to the same carbon atom of a cycloalkyl or heterocyclyl group may join together to form ═X¹, wherein each of the alkyl, alkenyl, alkynyl, aryl, heteroaryl, cycloalkyl, and heterocyclyl groups being a 1^{st} level substituent is optionally substituted by one or more 2^{nd} level substituents, wherein said 2^{nd} level substituent is, in each case, independently selected from the group consisting of C₁₋₆ alkyl, C₂₋₆ alkenyl, C₂₋₆alkynyl, 3- to 14-membered aryl, 3- to 14-membered heteroaryl, 3- to 14-membered cycloalkyl, 3- to 14-membered heterocyclyl, halogen, —CF₃, —CN, azido, —NO₂, —OR⁸¹, —N(R⁸²)(R⁸³), —S(O)₀₋₂R⁸¹, —S(O)₁₋₂OR⁸¹, —OS(O)₁₋₂R⁸¹, —OS(O)₁₋₂OR⁸¹, —S(O)₁₋₂N(R⁸²)(R⁸³), —OS(O)₁₋₂N(R⁸²)(R⁸³), —N(R⁸¹)S(O)₁₋₂R⁸¹, —NR⁸¹S(O)₁₋₂OR⁸¹, —NR⁸¹S(O)₁₋₂N(R⁸²)(R⁸³) —OP(O)(OR⁸¹)₂, —C(═X²)R⁸¹, —C(═X²)X²R⁸¹, —X²C(═X²)R⁸¹, and —X²C(═X²)X²R⁸¹, and/or any two 2^{nd} level substituents which are bound to the same carbon atom of a cycloalkyl or heterocyclyl group being a 1^{st} level substituent may join together to form ═X², wherein each of the C₁₋₆ alkyl, C₂₋₆ alkenyl, C₂₋₆ alkynyl, 3- to 14-membered aryl, 3- to 14-membered heteroaryl, 3- to 14-membered cycloalkyl, 3- to 14-membered heterocyclyl groups being a 2^{nd} level substituent is optionally substituted with one or more 3rd level substituents, wherein said 3^{rd} level substituent is, in each case, independently selected from the group consisting of C₁₋₃ alkyl, halogen, —CF₃, —CN, azido, —NO₂, —OH, —O(C₁₋₃ alkyl), —OCF₃, —S(C₁₋₃ alkyl), —NH₂, —NH(C₁₋₃ alkyl), —N(C₁₋₃ alkyl)₂, —NHS(O)₂(C₁₋₃ alkyl), —S(O)₂NH_{2-z}(C₁₋₃ alkyl)_{z}, —C(═O)OH, —C(═O)O(C₁₋₃ alkyl), —C(═O)NH_{2-z}(C₁₋₃ alkyl)_{z}, —NHC(═O)(C₁₋₃ alkyl), —NHC(═NH)NH_{z-2}(C₁₋₃ alkyl)_{z}, and —N(C₁₋₃ alkyl)C(═NH)NH_{2-z}(C₁₋₃ alkyl)_{z}, wherein each z is independently 0, 1, or 2 and each C₁₋₃ alkyl is independently methyl, ethyl, propyl or isopropyl, and/or any two 3^{rd} level substituents which are bound to the same carbon atom of a 3- to 14-membered cycloalkyl or heterocyclyl group being a 2^{nd} level substituent may join together to form ═O, ═S, ═NH, or ═N(C₁₋₃ alkyl);
wherein
each of R⁷¹, R⁷², and R⁷³ is independently selected from the group consisting of H, C₁₋₆ alkyl, C₂₋₆ alkenyl, C₂₋₆ alkynyl,
3- to 7-membered cycloalkyl, 5- or 6-membered aryl, 5- or 6-membered heteroaryl, and 3- to 7-membered heterocyclyl, wherein each of the C₁₋₆ alkyl, C₂₋₆ alkenyl, C₂₋₆ alkynyl, 3- to 7-membered cycloalkyl, 5- or 6-membered aryl, 5- or 6-membered heteroaryl, and 3- to 7-membered heterocyclyl groups is optionally substituted with one, two or three substituents independently selected from the group consisting of C₁₋₃ alkyl, halogen, —CF₃, —CN, azido, —NO₂, —OH, —O(C₁₋₃ alkyl), —OCF₃, ═O, —S(C₁₋₃ alkyl), —NH₂, —NH(C₁₋₃ alkyl), —N(C₁₋₃ alkyl)₂, —NHS(O)₂(C₁₋₃ alkyl), —S(O)₂NH_{2-z}(C₁₋₃ alkyl)_{z}, —C(═O)(C₁₋₃ alkyl), —C(═O)OH, —C(═O)O(C₁₋₃ alkyl), —C(═O)NH_{2-z}(C₁₋₃ alkyl)_{z}, —NHC(═O)(C₁₋₃ alkyl),
—NHC(═NH)NH_{z-2}(C₁₋₃ alkyl)_{z}, and —N(C₁₋₃ alkyl)C(═NH)NH_{2-z}(C₁₋₃ alkyl)_{z}, wherein each z is independently 0, 1, or 2 and each C₁₋₃ alkyl is independently methyl, ethyl, propyl or isopropyl;
each of R⁸¹, R⁸², and R⁸³ is independently selected from the group consisting of H, C₁₋₄ alkyl, C₂₋₄ alkenyl, C₂₋₄ alkynyl, 3- to 6-membered cycloalkyl, 5- or 6-membered aryl, 5- or 6-membered heteroaryl, and 3- to 6-membered heterocyclyl, wherein each of the C₁₋₄ alkyl, C₂₋₄ alkenyl, C₂₋₄ alkynyl, 3- to 6-membered cycloalkyl, 5- or 6-membered aryl, 5- or 6-membered heteroaryl, and 3- to 6-membered heterocyclyl groups is optionally substituted with one, two or three substituents independently selected from the group consisting of C₁₋₃ alkyl, halogen, —CF₃, —CN, azido, —NO₂, —OH, —O(C₁₋₃ alkyl), —OCF₃, ═O, —S(C₁₋₃ alkyl), —NH₂, —NH(C₁₋₃ alkyl), —N(C₁₋₃ alkyl)₂, —NHS(O)₂(C₁₋₃ alkyl), —S(O)₂NH_{2-z}(C₁₋₃ alkyl)_{z}, —C(═O)(C₁₋₃ alkyl), —C(═O)OH, —C(═O)O(C₁₋₃ alkyl), —C(═O)NH_{2-z}(C₁₋₃ alkyl)_{z}, —NHC(═O)(C₁₋₃ alkyl),
—NHC(═NH)NH_{z-2}(C₁₋₃ alkyl)_{z}, and —N(C₁₋₃ alkyl)C(═NH)NH_{2-z}(C₁₋₃ alkyl)_{z}, wherein each z is independently 0, 1, or 2 and
each C₁₋₃ alkyl is independently methyl, ethyl, propyl or isopropyl; and
each of X¹ and X² is independently selected from O, S, and N(R⁸⁴), wherein R⁸⁴ is H or C₁₋₃ alkyl.

2. The compound of claim 1, wherein R⁶ is substituted with one or more independently selected R⁷;
R⁷ is independently selected from the group consisting of alkyl, alkenyl, alkynyl, cycloalkyl, aryl, heterocyclyl, heteroaryl, halogen, —CN, azido, —NO₂, —OR¹¹, —N(R¹²)(R¹³), —N(R¹¹)(OR¹¹), —S(O)₀₋₂R¹¹, —S(O)₁₋₂OR¹¹, —OS(O)₁₋₂R¹¹, —OS(O)₁₋₂OR¹¹, —S(O)₁₋₂N(R¹²)(R¹³), —OS(O)₁₋₂N(R¹²)(R¹³), —N(R¹¹)S(O)₁₋₂R¹¹, —NR¹¹S(O)₁₋₂OR¹¹, —NR¹¹S(O)₁₋₂N(R¹²)(R¹³), —P(O)(OR¹¹)₂, —OP(O)(OR¹¹)₂, —C(═X)R¹¹, —C(═X)XR¹¹, —XC(═X)R¹¹, and —XC(═X)XR¹¹; and wherein each of the alkyl, alkenyl, alkynyl, cycloalkyl, aryl, heterocyclyl, and heteroaryl groups is optionally substituted with one or more independently selected R³⁰, and
wherein at least one of R⁷ is F and/or at least one of R⁷ is substituted with one or more F atoms.

3. The compound of claim 2, wherein at least one of R⁷ is F and/or at least one of R⁷ is C₁₋₃ alkyl, wherein the alkyl group of C₁₋₃ alkyl is substituted with one or more F atoms.

4. The compound of any one of claims 1 to 3:
wherein R⁶ is selected from the group consisting of 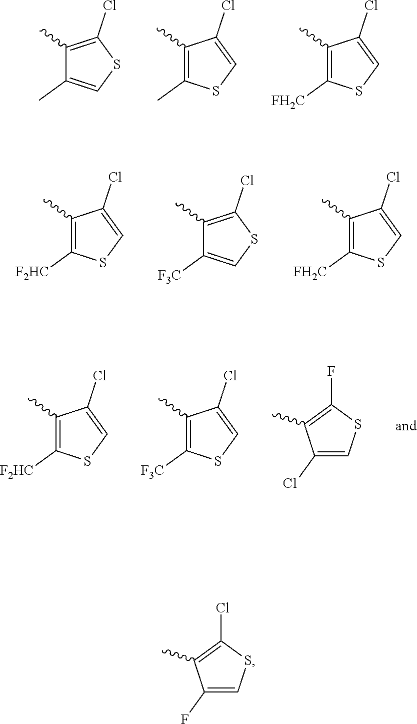
wherein [symbol] represents the bond by which R⁶ is bound to the remainder of the compound.

5. The compound of claim 4, wherein R⁶ is selected from the group consisting of 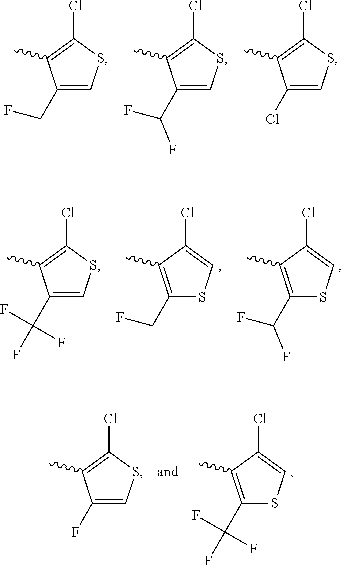
wherein [symbol] represents the bond by which R⁶ is bound to the remainder of the compound.

6. The compound of any of the claims 1 to 5, wherein R^{1a} is selected from the group consisting of C₁₋₃ alkyl, —O(C₁₋₃ alkyl), —S(C₁₋₃ alkyl), —NH(C₁₋₃ alkyl), piperazinyl, morpholinyl, piperidinyl, pyrrolidinyl, and azepanyl wherein each of the piperazinyl, morpholinyl, piperidinyl, pyrrolidinyl, and azepanyl groups is optionally substituted with one or two moieties independently selected from the group consisting of methyl, ethyl, —OH, —OCH₃, —SCH₃, cyclopropyl, 2-hydroxyethyl, 2-(N,N-dimethylamino)ethyl, 2-(N,N-dimethylamino)ethoxy, 2-aminoethyl, 2-(N-methylamino)ethyl, 2-(methoxy)ethyl, 4-methylpiperazinyl, —C(═O)(C₁₋₃ alkyl), —(CH₂)₁₋₃COOH, and —NH_{2-z}(CH₃)_{z}, wherein z is 0, 1, or 2; and each of the C₁₋₃ alkyl groups is optionally substituted with one or two moieties independently selected from the group consisting of —OH, —OCH₃, —SCH₃, cyclopropyl, piperazinyl, 4-methyl-piperazinyl, 4-(2-hydroxyethyl)piperazinyl, 2-(N,N-dimethylamino)ethoxy, and —NH_{2-z}(CH₃)_{z}, wherein z is 0, 1, or 2.

7. The compound of any of the claims 1 to 6, wherein R^{1a} is piperidinyl substituted with one to three R³⁰ independently selected from the group consisting of C₁ to C₄ alkyl.

8. The compound of any of the claims 1 to 7, wherein R^{1a} is piperidinyl substituted with one to three methyl.

9. The compound of any of the claims 1 to 8, wherein Q is C₆₋₁₀ aryl, wherein the C₆₋₁₀ aryl is optionally substituted with one or more R³⁰ being —OR¹¹, wherein R¹¹ is independently selected from C₁₋₈ alkyl.

10. The compound of any of the claims 1 to 9, wherein Q is C₆ aryl, wherein the C₆ aryl is optionally substituted with one or more R³⁰ being —OR¹¹, wherein R¹¹ is methyl.

11. The compound of any of the claims 1 to 10, wherein R³ is selected from the group consisting of methyl, ethyl, phenyl, and pyridyl, wherein each of the methyl, ethyl, phenyl, and pyridyl is optionally substituted with one or more R³⁰ being methoxy.

12. The compound of any of the claims 1 to 11, wherein R³ is selected from the group consisting of methyl, 2-methoxyethyl, methoxyphenyl, and 3-methoxypyridyl, and 1,3-dimethoxyphenyl.

13. The compound of any of the claims 1 to 12, wherein R¹ and R³ join together via a group L′ to form a moiety R¹-L′-R³ and, optionally, wherein R³ is a bond.

14. The compound of claim 13, wherein R¹ and R³ join together via a group L′ to form a moiety Q-L′-R³, and optionally, wherein R³ is a bond.

15. The compound of any of the claims 1 to 14,
wherein R⁶ is substituted with two independently selected R⁷.

16. The compound of claim 15, wherein R⁶ is substituted with two R⁷ that differ from each other.

17. The compound of any of the claims 1 to 16, wherein R⁷ is independently selected from the group consisting of C₁₋₃ alkyl, halogen, —CN, —O(C₁₋₃ alkyl), —NH(C₁₋₃ alkyl), and —N(C₁₋₃ alkyl)₂.

18. The compound of any of the claims 1 to 17, wherein any two R⁷ which are bound to the same atom of R⁶ being a heterocyclyl group may join together to form ═0, wherein each of the C₁₋₃ alkyl groups is optionally substituted with one or more independently selected R³⁰.

19. The compound of any of the claims 1 to 18, wherein R⁷ is independently selected from the group consisting of halogen and C₁₋₂ alkyl, wherein the C₁₋₂ alkyl groups is optionally substituted with one, two, or three independently selected R³⁰.

20. The compound of any of the claims 1 to 19, wherein R⁷ is independently selected from the group consisting of Cl, F, methyl, fluoromethyl, difluoromethyl, and trifluoromethyl.

21. The compound of any of the claims 1 to 20, wherein R¹ and R³ join together via a group L′ to form a moiety R¹-L′-R³ and wherein L′ is selected from the group consisting of C₃₋₁₀ alkylene, C₃₋₁₀ alkenylene, C₃₋₁₀ alkynylene, —(CH₂)ₚ—[Y—(CH₂)_{q}]ᵣ—, and alkenylene —[Y—(CH₂)_{q}]ᵣ—, wherein p is an integer between 1 and 10, q is an integer between 0 and 6, r is an integer between 1 and 3, wherein if q is 0 then r is 1; Y is independently selected from 0, S, and —N(R¹³)—; and each of the C₃₋₁₀ alkylene, C₃₋₁₀ alkenylene, C₃₋₁₀ alkynylene, —(CH₂)ₚ—, and —(CH₂)_{q}— groups is optionally substituted with one or more independently selected R³⁰; optionally wherein R¹ and R³ join together via a group L′ to form a moiety Q-L′-R³; and, optionally:
wherein L′ is selected from the group consisting of 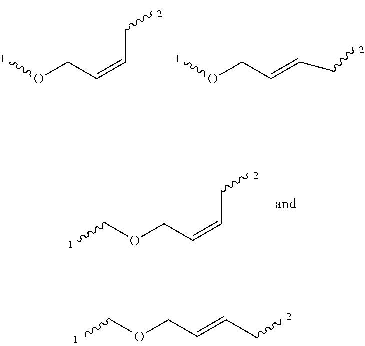
wherein 1 [symbol] represents the bond by which L′ is bound to R¹, and [symbol] 2 represents the bond by which L′ is bound to R; and/or
wherein R³ is a bond.

22. The compound of claim 1, wherein:
R^{1a} is selected from the group consisting of piperidinyl substituted with one to three moieties independently selected from the group consisting of C₁ to C₄ alkyl; piperazinyl group substituted with one or two moieties independently selected from the group consisting of 2-hydroxyethyl and C₁ to C₄ alkyl; azepanyl substituted with one to three moieties independently selected from the group consisting of C₁ to C₄ alkyl; morpholinyl; and C₁₋₃ alkyl group substituted with —NH_{2-z}(CH₃)_{z}, wherein z is 0, 1, or 2;
Q is C₆₋₁₀ aryl, wherein the C₆₋₁₀ aryl is optionally substituted with one or more R³⁰ being —OR¹¹, wherein R¹¹ is independently selected from C₁₋₁₂ alkyl;
E is O;
R³ is selected from the group consisting of C₁₋₆ alkyl, C₆ aryl, and 5- or 6-membered heteroaryl, wherein each of the C₁₋₆ alkyl, C₆ aryl, and 5- or 6-membered heteroaryl groups is optionally substituted with one or more R³⁰ independently selected from the group consisting of C₁₋₆ alkyl, and OR¹¹, wherein R¹¹ C₁₋₁₂ alkyl;
both R^{4a} and R^{4b} are are H;
R⁶ is thienyl substituted with one, two, or three independently selected R⁷;
R⁷ is independently selected from the group consisting of Cl, F, methyl, fluoromethyl, difluoromethyl, and trifluoromethyl, preferably wherein at least one of R⁷ is F and/or at least one of R⁷ is substituted with one or more F atoms; and
L is a bond; and
L′, if present, is C₃₋₁₀ alkenylene-[O—(CH₂)_{q}]ᵣ—.

23. The compound of claim 1, wherein:
R^{1a} is selected from the group consisting of 1-methylpiperidinyl, 1,2-dimethylpiperidinyl, 1,2,6-trimethylpiperidinyl, 1-methylazepanyl, 4-(2-hydroxyethyl)piperazinyl, 1-methyl-4-(2-hydroxyethyl)piperazinyl, 4-methylpiperazinyl, 4-acetylpiperazinyl, and (2-hydroxyethyl)amino;
Q is C₆ aryl, wherein the C₆ aryl is optionally substituted with one or more —OR¹¹, wherein R¹¹ is methyl;
E is O;
R³ is selected from the group consisting of methyl, 2-methoxyethyl, methoxyphenyl, and 3-methoxypyridyl, and 1,3-dimethoxyphenyl;
both R^{4a} and R^{4b} are H;
R⁶ is selected from the group consisting of 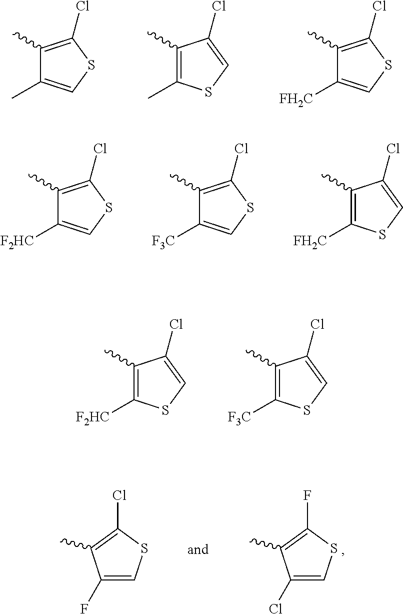
wherein [symbol] represents the bond by which R⁶ is bound to the remainder of the compound;
L is a bond; and [symbol]
L′, if present, is selected from the group consisting of 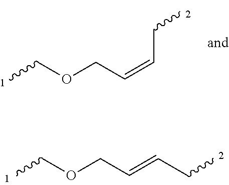 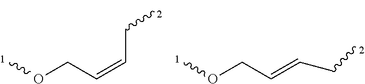
wherein 1 [symbol] represents the bond by which L′ is bound to R¹, and [symbol] 2 represents the bond by which L′ is bound to R³.

24. The compound of claim 22 or 23, wherein R⁶ is selected from the group consisting of: 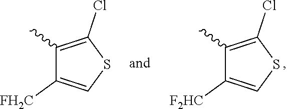
wherein [symbol] represents the bond by which R⁶ is bound to the remainder of the compound; and/or
wherein R¹ and R³ join together via a group L′ to form a moiety R¹-L′-R³, preferably to form a moiety Q-L′-R³, and optionally wherein R³ is a bond.

25. The compound of claim 1, selected from the group consisting of 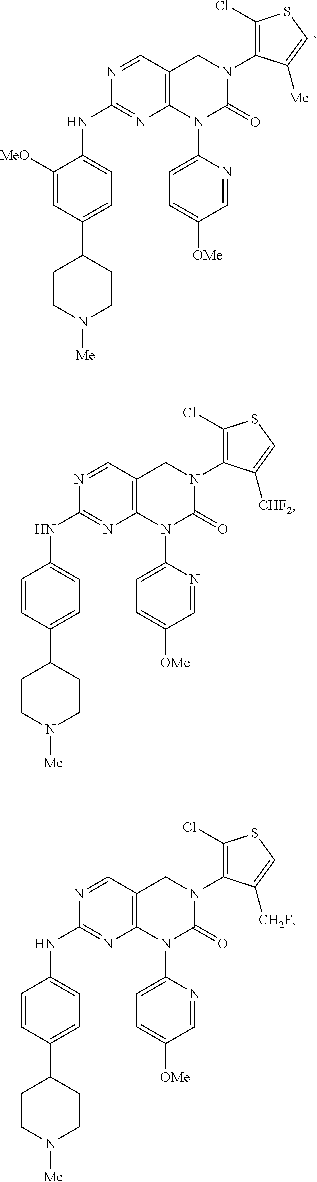 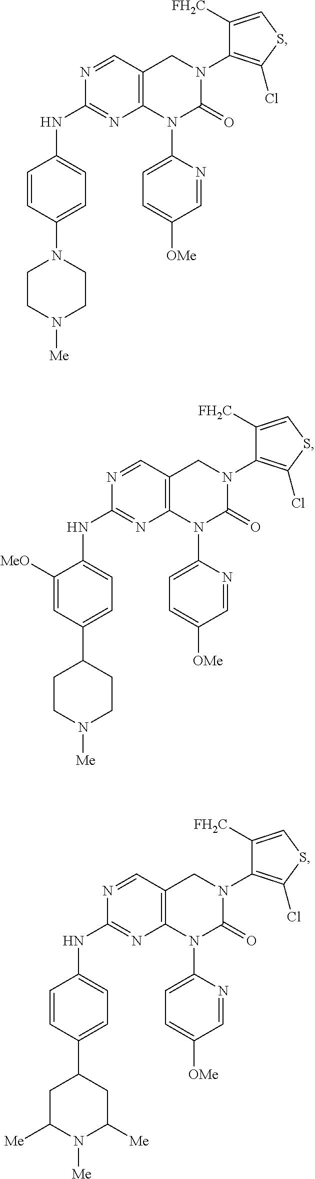 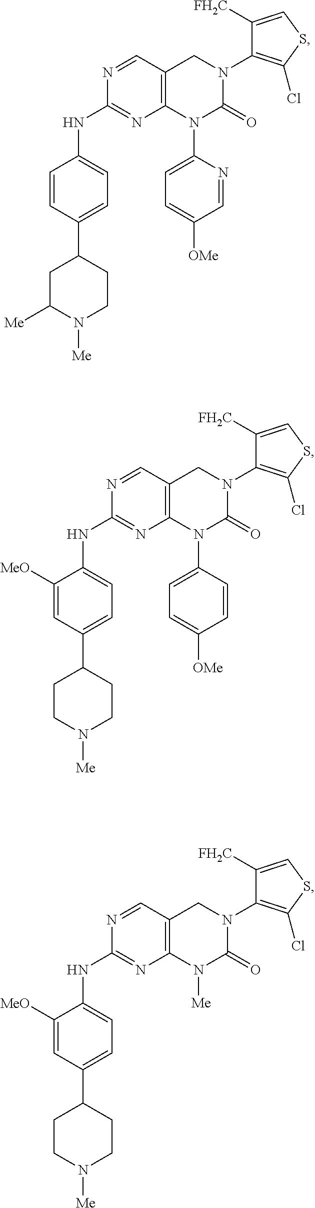 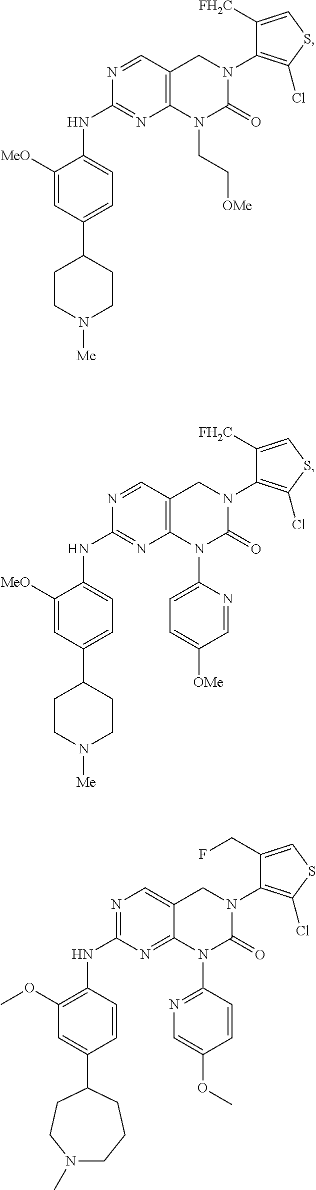 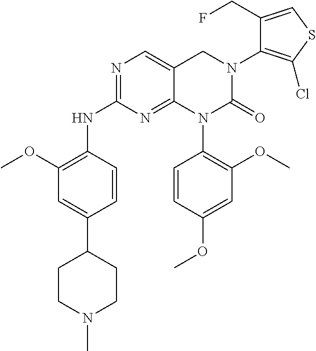 [SMILES: COc1ccc(N2C(=O)N(c3c(CF)csc3Cl)Cc3cnc(Nc4ccc(C5CCN(C)CC5)cc4OC)nc32)c(OC)c1]
and solvates, salts, N-oxides, complexes, polymorphs, crystalline forms, racemic mixtures, diastereomers, enantiomers, tautomers, conformers, isotopically labeled forms, prodrugs, and combinations thereof.

26. A compound selected from the group consisting of a kinase inhibitor of the formula (I): 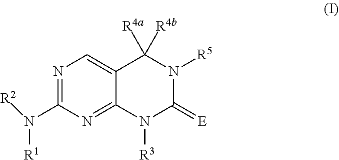
and solvates, salts, N-oxides, complexes, polymorphs, crystalline forms, racemic mixtures, diastereomers, enantiomers, tautomers, conformers, isotopically labeled forms, prodrugs, and combinations thereof;
wherein:
R¹ is -Q-R^{1a};
R^{1a} is selected from the group consisting of H, alkyl, alkenyl, alkynyl, cycloalkyl, aryl, heterocyclyl, heteroaryl, halogen, —CN, azido, —NO₂, —OR¹¹, —N(R¹²)(R¹³), —N(R¹¹)(OR¹), —S(O)₀₋₂R¹¹, —S(O)₁₋₂OR¹¹, —OS(O)₁₋₂R¹¹, —OS(O)₁₋₂OR¹¹, —S(O)₁₋₂N(R¹²)(R¹³), —OS(O)₁₋₂N(R¹²)(R¹³), —N(R¹¹)S(O)₁₋₂R¹¹, —NR¹¹S(O)₁₋₂OR¹¹, —NR¹¹S(O)₁₋₂N(R¹²)(R¹³), —P(O)(OR¹¹)₂,
—OP(O)(OR¹¹)₂, —C(═X)R¹¹, —C(═X)XR¹¹, —XC(═X)R¹¹, and —XC(═X)XR¹¹, wherein each of the alkyl, alkenyl, alkynyl, cycloalkyl, aryl, heterocyclyl, and heteroaryl groups is optionally substituted with one or more independently selected R³⁰;
Q is selected from the group consisting of a bond, alkyl, alkenyl, alkynyl, cycloalkyl, aryl, heterocyclyl, heteroaryl, wherein each of the alkyl, alkenyl, alkynyl, cycloalkyl, aryl, heterocyclyl, and heteroaryl groups is optionally substituted with one or more independently selected R³⁰;
R² is H;
R³ is selected from the group consisting of a bond, H, alkyl, alkenyl, alkynyl, cycloalkyl, aryl, heterocyclyl, heteroaryl, halogen, —CN, azido, —NO₂, —OR¹¹, —N(R¹²)(R¹³), —N(R¹¹)(OR¹), —S(O)₀₋₂R¹¹, —S(O)₁₋₂OR¹¹, —OS(O)₁₋₂ R¹¹, —OS(O)₁₋₂OR¹¹, —S(O)₁₋₂N(R¹²)(R¹³), —OS(O)₁₋₂N(R¹²)(R¹³), —N(R¹¹)S(O)₁₋₂R¹¹, —NR¹¹S(O)₁₋₂OR¹¹, —NR¹¹S(O)₁₋₂N(R¹²)(R¹³), —P(O)(OR¹¹)₂, —OP(O)(OR¹¹)₂, —C(═X)R¹¹, —C(═X)XR¹¹, —XC(═X)R¹¹, and —XC(═X)XR¹¹, wherein each of the alkyl, alkenyl, alkynyl, cycloalkyl, aryl, heterocyclyl, and heteroaryl groups is optionally substituted with one or more independently selected R³⁰;
optionally R¹ and R³ may join together via a group L′ to form a moiety R¹-L′-R³, preferably to form a moiety Q-L′-R³, wherein L′ is selected from the group consisting of a bond, alkylene, alkenylene, alkynylene, —(CH₂)ₚ—[Y—(CH₂)_{q}]ᵣ—, alkenylene —[Y—(CH₂)_{q}]ᵣ—, wherein p is an integer between 1 and 10, q is an integer between 0 and 6, r is an integer between 1 and 3, wherein if q is 0 then r is 1; Y is independently selected from O, S, and —N(R¹³)—; and each of the alkylene, alkenylene, alkynylene, —(CH₂)ₚ—, and —(CH₂)_{q}— groups is optionally substituted with one or more independently selected R³⁰;
each of R^{4a} and R^{4b} is independently selected from the group consisting of H, C₁₋₈ alkyl, and —(CH₂)ₛ—[O—(CH₂)ₜ]ᵤ—H wherein s is an integer between 0 and 7, t is an integer between 0 and 7, u is an integer between 1 and 3, wherein if t is 0 then u is 1, and the total number of carbon and oxygen atoms of the —(CH₂)ₛ—[O—(CH₂)ₜ]ᵤ—H does not exceed 8; or optionally R^{4a} and R^{4b} may join together to form, together with the carbon to which they are attached, C₃₋₈ cycloalkyl or a 4- to 8-membered heterocyclyl comprising at least one O as the only heteroatom element, wherein in case that the 4- to 8-membered heterocyclyl comprises more than one O, different O are not directly bound to each other;
R⁵ is -L-R⁶;
L is selected from the group consisting of a bond, C₁₋₆ alkylene, C₂₋₆ alkenylene, C₂₋₆ alkynylene, and —(CH₂)ₘ—[Y—(CH₂)ₙ]ₒ—, wherein m is an integer between 1 and 6, n is an integer between 0 and 3, o is an integer between 1 and 3, wherein if n is 0 then o is 1; Y is independently selected from 0, S, and —N(R¹³)—; and
each of the C₁₋₆ alkylene, C₂₋₆ alkenylene, C₂₋₆ alkynylene, —(CH₂)ₘ—, and —(CH₂)ₙ— groups is optionally substituted with one or two independently selected R³⁰;
R⁶ is heteroaryl or heterocyclyl each of which is optionally substituted with one or more independently selected R⁷;
R⁷ is independently selected from the group consisting of alkyl, alkenyl, alkynyl, cycloalkyl, aryl, heterocyclyl, heteroaryl, halogen, —CN, azido, —NO₂, —OR¹¹, —N(R¹²)(R¹³), —N(R¹¹)(OR¹¹), —S(O)₀₋₂R¹¹, —S(O)₁₋₂OR¹¹, —OS(O)₁₋₂R¹¹,
—OS(O)₁₋₂OR¹¹, —S(O)₁₋₂N(R¹²)(R¹³), —OS(O)₁₋₂N(R¹²)(R¹³), —N(R¹¹)S(O)₁₋₂R¹¹, —NR¹¹S(O)₁₋₂OR¹¹, —NR¹¹S(O)₁₋₂N(R¹²)(R¹³), —P(O)(OR¹¹)₂, —OP(O)(OR¹¹)₂, —C(═X)R¹¹, —C(═X)XR¹¹, —XC(═X)R¹¹, and —XC(═X)XR¹¹, and/or any two R⁷ which are bound to the same atom of R⁶ being a heterocyclyl group may join together to form ═O, wherein each of the alkyl, alkenyl, alkynyl, cycloalkyl, aryl, heterocyclyl, and heteroaryl groups is optionally substituted with one or more independently selected R³⁰;
E is O or S;
R¹¹ is independently selected from the group consisting of H, alkyl, alkenyl, alkynyl, cycloalkyl, aryl, heteroaryl, and heterocyclyl, wherein each of the alkyl, alkenyl, alkynyl, cycloalkyl, aryl, heteroaryl, and heterocyclyl groups is optionally substituted with one or more independently selected R³⁰;
each of R¹² and R¹³ is independently selected from the group consisting of H, alkyl, alkenyl, alkynyl, cycloalkyl, aryl, heteroaryl, and heterocyclyl, or R¹² and R¹³ may join together with the nitrogen atom to which they are attached to form the group —N═CR¹⁵R¹⁶, wherein each of the alkyl, alkenyl, alkynyl, cycloalkyl, aryl, heteroaryl, and heterocyclyl
groups is optionally substituted with one or more independently selected R³⁰;
each of R¹⁵ and R¹⁶ is independently selected from the group consisting of H, alkyl, alkenyl, alkynyl, cycloalkyl, aryl, heteroaryl, heterocyclyl, and —NH_{y}R²⁰_{2-y}, or R¹⁵ and R¹⁶ may join together with the atom to which they are attached to form a ring which is optionally substituted with one or more independently selected R³⁰, wherein each of the alkyl, alkenyl, alkynyl, cycloalkyl, aryl, heteroaryl, and heterocyclyl groups is optionally substituted with one or more independently selected R³⁰;
y is an integer from 0 to 2;
R²⁰ is independently selected from the group consisting of alkyl, alkenyl, alkynyl, cycloalkyl, aryl, heteroaryl, and heterocyclyl, wherein each of the alkyl, alkenyl, alkynyl, cycloalkyl, aryl, heteroaryl, and heterocyclyl groups is optionally substituted with one or more independently selected R³⁰; and
R³⁰ is a 1^{st} level substituent and is, in each case, independently selected from the group consisting of alkyl, alkenyl, alkynyl, aryl, heteroaryl, cycloalkyl, heterocyclyl, halogen, —CN, azido, —NO₂, —OR⁷¹, —N(R⁷²)(R⁷³), —S(O)₀₋₂R⁷¹, —S(O)₁₋₂OR⁷¹, —OS(O)₁₋₂R⁷¹, —OS(O)₁₋₂OR⁷¹, —S(O)₁₋₂N(R⁷²)(R⁷³), —OS(O)₁₋₂N(R⁷²)(R⁷³), —N(R⁷¹)S(O)₁₋₂R⁷¹, —NR⁷¹S(O)₁₋₂OR⁷¹, —NR⁷¹S(O)₁₋₂N(R⁷²)(R⁷³), —OP(O)(OR⁷¹)₂, —C(═X¹)R⁷¹, —C(═X¹)X¹R⁷¹, —X¹C(═X¹)R⁷¹, and —X¹C(═X¹)X¹R⁷¹, and/or any two R³⁰ which are bound to the same carbon atom of a cycloalkyl or heterocyclyl group may join together to form ═X¹, wherein each of the alkyl, alkenyl, alkynyl, aryl, heteroaryl, cycloalkyl, and heterocyclyl groups being a 1^{st} level substituent is optionally substituted by one or more 2^{nd} level substituents, wherein said 2^{nd} level substituent is, in each case, independently selected from the group consisting of C₁₋₆ alkyl, C₂₋₆ alkenyl, C₂₋₆alkynyl, 3- to 14-membered aryl, 3- to 14-membered heteroaryl, 3- to 14-membered cycloalkyl, 3- to 14-membered heterocyclyl, halogen, —CF₃, —CN, azido, —NO₂, —OR⁸¹, —N(R⁸²)(R⁸³), —S(O)₀₋₂R⁸¹, —S(O)₁₋₂OR⁸¹, —OS(O)₁₋₂R⁸¹, —OS(O)₁₋₂OR⁸¹, —S(O)₁₋₂N(R⁸²)(R⁸³), —OS(O)₁₋₂N(R⁸²)(R⁸³), —N(R⁸¹)S(O)₁₋₂R⁸¹, —NR⁸¹S(O)₁₋₂₀R⁸¹, —NR⁸¹S(O)₁₋₂N(R⁸²)(R⁸³), —OP(O)(OR⁸¹)₂, —C(═X²)R⁸¹, —C(═X²)X²R⁸¹, —X²C(═X²)R⁸¹, and —X²C(═X²)X²R⁸¹, and/or any two 2^{nd} level substituents which are bound to the same carbon atom of a cycloalkyl or heterocyclyl group being a 1^{st} level substituent may join together to form ═X², wherein each of the C₁₋₆ alkyl, C₂₋₆ alkenyl, C₂₋₆ alkynyl, 3- to 14-membered aryl, 3- to 14-membered heteroaryl, 3- to 14-membered cycloalkyl, 3- to 14-membered heterocyclyl groups being a 2^{nd} level substituent is optionally substituted with one or more 3rd level substituents, wherein said 3^{rd} level substituent is, in each case, independently selected from the group consisting of C₁₋₃ alkyl, halogen, —CF₃, —CN, azido, —NO₂, —OH, —O(C₁₋₃ alkyl), —OCF₃, —S(C₁₋₃ alkyl), —NH₂, —NH(C₁₋₃ alkyl), —N(C₁₋₃ alkyl)₂, —NHS(O)₂(C₁₋₃ alkyl), —S(O)₂NH_{2-z}(C₁₋₃ alkyl)_{z}, —C(═O)OH, —C(═O)O(C₁₋₃ alkyl), —C(═O)NH_{2-z}(C₁₋₃ alkyl)_{z}, —NHC(═O)(C₁₋₃ alkyl), —NHC(═NH)NH_{z-2}(C₁₋₃ alkyl)_{z}, and —N(C₁₋₃ alkyl)C(═NH)NH_{2-z}(C₁₋₃ alkyl)_{z}, wherein each z is independently 0, 1, or 2 and each C₁₋₃ alkyl is independently methyl, ethyl, propyl or isopropyl, and/or any two 3^{rd} level substituents which are bound to the same carbon atom of a 3- to 14-membered cycloalkyl or heterocyclyl group being a 2^{nd} level substituent may join together to form ═O, ═S, ═NH, or ═N(C₁₋₃ alkyl); wherein
each of R⁷¹, R⁷², and R⁷³ is independently selected from the group consisting of H, C₁₋₆ alkyl, C₂₋₆ alkenyl, C₂₋₆ alkynyl,
3- to 7-membered cycloalkyl, 5- or 6-membered aryl, 5- or 6-membered heteroaryl, and 3- to 7-membered heterocyclyl, wherein each of the C₁₋₆ alkyl, C₂₋₆ alkenyl, C₂₋₆ alkynyl, 3- to 7-membered cycloalkyl, 5- or 6-membered aryl, 5- or 6-membered heteroaryl, and 3- to 7-membered heterocyclyl groups is optionally substituted with one, two or three substituents independently selected from the group consisting of C₁₋₃ alkyl, halogen, —CF₃, —CN, azido, —NO₂, —OH, —O(C₁₋₃ alkyl), —OCF₃, ═O, —S(C₁₋₃ alkyl), —NH₂, —NH(C₁₋₃ alkyl), —N(C₁₋₃ alkyl)₂, —NHS(O)₂(C₁₋₃ alkyl), —S(O)₂NH_{2 -z}(C₁₋₃ alkyl)_{z}, —C(═O)(C₁₋₃ alkyl), —C(═O)OH, —C(═O)O(C₁₋₃ alkyl), —C(═O)NH_{2-z}(C₁₋₃ alkyl)_{z}, —NHC(═O)(C₁₋₃ alkyl),
—NHC(═NH)NH_{z-2}(C₁₋₃ alkyl)_{z}, and —N(C₁₋₃ alkyl)C(═NH)NH_{2-z}(C₁₋₃ alkyl)_{z}, wherein each z is independently 0, 1, or 2 and each C₁₋₃ alkyl is independently methyl, ethyl, propyl or isopropyl;
each of R⁸¹, R⁸², and R⁸³ is independently selected from the group consisting of H, C₁₋₄ alkyl, C₂₋₄ alkenyl, C₂₋₄ alkynyl, 3- to 6-membered cycloalkyl, 5- or 6-membered aryl, 5- or 6-membered heteroaryl, and 3- to 6-membered heterocyclyl, wherein each of the C₁₋₄ alkyl, C₂₋₄ alkenyl, C₂₋₄ alkynyl, 3- to 6-membered cycloalkyl, 5- or 6-membered aryl, 5- or 6-membered heteroaryl, and 3- to 6-membered heterocyclyl groups is optionally substituted with one, two or three substituents independently selected from the group consisting of C₁₋₃ alkyl, halogen, —CF₃, —CN, azido, —NO₂, —OH, —O(C₁₋₃ alkyl), —OCF₃, ═O, —S(C₁₋₃ alkyl), —NH₂, —NH(C₁₋₃ alkyl), —N(C₁₋₃ alkyl)₂, —NHS(O)₂(C₁₋₃ alkyl), —S(O)₂NH_{2-z}(C₁₋₃ alkyl)_{z}, —C(═O)(C₁₋₃ alkyl), —C(═O)OH, —C(═O)O(C₁₋₃ alkyl), —C(═O)NH_{2-z}(C₁₋₃ alkyl)_{z}, —NHC(═O)(C₁₋₃ alkyl),
—NHC(═NH)NH_{z-2}(C₁₋₃ alkyl)_{z}, and —N(C₁₋₃ alkyl)C(═NH)NH_{2-z}(C₁₋₃ alkyl)_{z}, wherein each z is independently 0, 1, or 2 and
each C₁₋₃ alkyl is independently methyl, ethyl, propyl or isopropyl; and
each of X¹ and X² is independently selected from O, S, and N(R⁸⁴), wherein R⁸⁴ is H or C₁₋₃ alkyl;
with the proviso that:
when E is O and either of (1) or (2) is true:
(1) R¹ and R³ do not join together via a group L′ to form a moiety R¹-L′-R³; or
(2) both of R^{4a} and R^{4b} are H,
then either:
(a) R⁶ is: (i) a 5-membered monocyclic heteroaryl which contains at least one S ring atom and which is substituted with one or more independently selected R⁷; (ii) a 5-membered monocyclic heteroaryl which contains at least two nitrogen atoms and which is substituted with one or more independently selected R⁷; or (iii) a 5-membered monocyclic heteroaryl which contains at least one nitrogen atom and at least one oxygen atom and which is substituted with one or more independently selected R⁷; or
(b) R⁷ is independently selected from the group consisting of alkyl, alkenyl, alkynyl, cycloalkyl, aryl, heterocyclyl, heteroaryl, halogen, —CN, azido, —NO₂, —OR¹¹, —N(R¹²)(R¹³), —N(R¹¹)(OR¹¹), —S(O)₀₋₂R¹¹, —S(O)₁₋₂OR¹¹, —OS(O)₁₋₂R¹¹, —OS(O)₁₋₂OR¹¹, —S(O)₁₋₂N(R¹²)(R¹³), —OS(O)₁₋₂N(R¹²)(R¹³), —N(R¹¹)S(O)₁₋₂R¹¹, —NR¹¹S(O)₁₋₂OR¹¹, —NR¹¹S(O)₁₋₂N(R¹²)(R¹³), —P(O)(OR¹¹)₂, —OP(O)(OR¹¹)₂, —C(═X)R¹¹, —C(═X)XR¹¹, —XC(═X)R¹¹, and —XC(═X)XR¹¹, wherein each of the alkyl, alkenyl, alkynyl, cycloalkyl, aryl, heterocyclyl, and heteroaryl groups is optionally substituted with one or more independently selected R³⁰, wherein at least one of R⁷ is F and/or at least one of R⁷ is substituted with one or more F atoms.

27. The compound of claim 26, wherein R⁶ is a 5-membered monocyclic heteroaryl which contains at least one S ring atom and which is substituted with one or more independently selected R⁷;
R⁷ is independently selected from the group consisting of alkyl, alkenyl, alkynyl, cycloalkyl, aryl, heterocyclyl, heteroaryl, halogen, —CN, azido, —NO₂, —OR¹¹, —N(R¹²)(R¹³), —N(R¹¹)(OR¹¹), —S(O)₀₋₂R¹¹, —S(O)₁₋₂OR¹¹, —OS(O)₁₋₂R¹¹, —OS(O)₁₋₂OR¹¹, —S(O)₁₋₂N(R¹²)(R¹³), —OS(O)₁₋₂N(R¹²)(R¹³), —N(R¹¹)S(O)₁₋₂R¹¹, —NR¹¹S(O)₁₋₂OR¹¹, —NR¹¹S(O)₁₋₂N(R¹²)(R¹³), —P(O)(OR¹¹)₂, —OP(O)(OR¹¹)₂, —C(═X)R¹¹, —C(═X)XR¹¹, —XC(═X)R¹¹, and —XC(═X)XR¹¹; and wherein each of the alkyl, alkenyl, alkynyl, cycloalkyl, aryl, heterocyclyl, and heteroaryl groups is optionally substituted with one or more independently selected R³⁰, and
wherein at least one of R⁷ is F and/or at least one of R⁷ is substituted with one or more F atoms.

28. The compound of claim 27, wherein at least one of R⁷ is F and/or at least one of R⁷ is C₁₋₃ alkyl, wherein the alkyl group of C₁₋₃ alkyl is substituted with one or more F atoms.

29. The compound of any one of claims 1 to 28, wherein the compound is in substantially pure form, in particular in greater than about 90%, 95%, 98% or 99% pure form.

30. A pharmaceutical composition comprising the compound of any one of claims 1 to 29, and optionally further comprising a pharmaceutically acceptable excipient.

31. A method for the treatment of a disease, disorder or condition in a subject, comprising administering to the subject a compound of any one of claims 1 to 29, or a pharmaceutical composition of claim 30 optionally wherein the disease, disorder or condition is associated with a kinase, such as one or more disclosed herein.

32. A compound for use, or a pharmaceutical composition for use, in a treatment of a proliferative disorder in a subject, the treatment comprising administering the compound or the pharmaceutical composition to the subject, wherein the compound is selected from any one of claims 1 to 29, and the pharmaceutical composition is a pharmaceutical composition of claim 30, optionally wherein the disease, disorder or condition is associated with a kinase, such as one or more disclosed herein.

33. The compound for use, or the pharmaceutical composition for use, of claim 32, wherein the proliferative disorder is a cancer or tumor.

34. The compound for use, or pharmaceutical composition for use, of claim 32 or 33, wherein the proliferative disorder is characterised by, or cells involved with the proliferative disorder characterised by:
(i) the presence of a human chromosomal translocation at 11q23; (ii) the presence of a rearrangement of the KMT2A gene; and/or (iii) the presence of an KMT2A fusion oncoprotein, preferably wherein:
(a) the human chromosome translocation is one selected from the group consisting of: t(4,11), t(9,11), t(11,19), t(10,11) and t(6,11); and/or
(b) the rearrangement of the KMT2A gene comprises, or the KMT2A fusion oncoprotein is expressed from a rearrangement that comprises, a fusion of the KMT2A gene with a translocation partner gene selected from the group consisting of: AF4, AF9, ENL, AF10, ELL and AF6; and/or
wherein the proliferative disorder is: (i) a myeloma, preferably multiple myeloma; or (ii) a leukaemia, preferably an acute myeloid leukaemia (AML) or an acute lymphoblastic leukaemia (ALL), more preferably T cell acute lymphoblastic leukaemia (T-ALL), an MLL-AML or an MLL-ALL; and/or
wherein the subject is a human paediatric patient and/or is a subject carrying a KMT2A rearrangement (KMT2A-r); preferably wherein the subject is a patient suffering from a KMT2A-r leukaemia.

35. A method for determining that a subject suffering from a proliferative disorder is suitable for treatment with a compound or pharmaceutical composition as defined in claim 32 or 33, the method comprising, determining in a biological sample that has been obtained from said subject, and preferable that comprises cells involved with the proliferative disorder:
(X) the presence of MEF2C protein, such as of phosphorylated MEF2C protein and/or of MEF2C protein as an active transcription factor; preferably wherein the proliferative disorder is further characterised by the presence of phosphorylated HDAC4 protein, such as of HDAC4 protein phosphorylated by SIK3; and/or
(Y) (i) the presence of a human chromosomal translocation at 11q23; (ii) the presence of a rearrangement of the KMT2A gene; (iii) the presence of an KMT2A fusion oncoprotein; and/or (iv) the presence of a mutation in the KRAS gene and/or in the RUNX1 gene,
wherein, the presence of said protein, translocation, rearrangement, oncoprotein and or mutation in the biological sample indicates that the subject is suitable for treatment with the compound or pharmaceutical composition; and, optionally:
the method comprising determining in a biological sample that has been obtained from said subject:
(i) the presence of a human chromosomal translocation at 11q23; (ii) the presence of a rearrangement of the KMT2A gene; and/or (iii) the presence of an KMT2A fusion oncoprotein, preferably wherein:
(a) the human chromosome translocation is one selected from the group consisting of: t(4,11), t(9,11), t(11,19), t(10,11), and t(6,11); and/or
(b) the rearrangement of the KMT2A gene comprises, or the KMT2A fusion oncoprotein is expressed from a rearrangement that comprises, a fusion of the KMT2A gene with a translocation partner gene selected from the group consisting of: AF4, AF9, ENL, AF10, ELL and AF6; and/or
wherein the proliferative disorder is: (i) a myeloma, preferably multiple myeloma; or (ii) a leukaemia, preferably an acute myeloid leukaemia (AML) or an acute lymphoblastic leukaemia (ALL), more preferably T cell acute lymphoblastic leukaemia (T-ALL), an MLL-AML or an MLL-ALL; and/or
wherein the subject is a human paediatric patient and/or is a subject carrying a KMT2A rearrangement (KMT2A-r); preferably wherein such subject is a patient suffering from a KMT2A-r leukaemia.

36. A method of preparing a compound of claim 29, comprising the steps:
providing a compound of any one of claims 1 to 28 in admixture with one or more impurities; and
removing at least a fraction of the impurities from the admixture.

37. A method of manufacturing a pharmaceutical composition comprising the step of formulating a compound of any one of claims 1 to 29 together with a pharmaceutically acceptable excipient.

38. A method of preparing a pharmaceutical package, comprising the steps:
inserting into packaging a pharmaceutical composition of claim 30, preferably wherein the pharmaceutical composition is in finished pharmaceutical form, thereby forming a package containing the pharmaceutical composition; and optionally,
inserting into the package a leaflet describing prescribing information for the pharmaceutical composition.

39. A pharmaceutical package containing a pharmaceutical composition of claim 30, preferably, wherein the pharmaceutical composition is in finished pharmaceutical form.
